(19) United States
(12) Patent Application Publication
(10) Pub. No.: US 2024/0083853 A1
(43) Pub. Date: Mar. 14, 2024

(54) HSD17B13 INHIBITORS AND USES THEREOF

(71) Applicant: FL2022-001, Inc., San Francisco, CA (US)

(72) Inventors: Steven P. Govek, San Diego, CA (US); Andiliy G. Lai, San Diego, CA (US); Andrew R. Hudson, San Diego, CA (US); Nicholas D. Smith, San Diego, CA (US); Karensa L. Fasanya, San Diego, CA (US)

(21) Appl. No.: 18/247,119

(22) PCT Filed: Sep. 29, 2021
(86) PCT No.: PCT/US2021/052645

§ 371 Date: Mar. 29, 2023

Related U.S. Application Data
(60) Provisional application No. 63/085,843, filed on Sep. 30, 2020.

(51) Int. Cl. C07D 231/56; C07D 401/10; C07D 403/04; C07D 471/04; C07D 249/18; C07D 235/26; C07D 491/107; C07D 413/10; C07D 403/10; C07D 487/08; C07D 401/14; C07D 401/12; C07D 417/10; C07D 495/10

(52) U.S. Cl. CPC C07D 231/56; C07D 401/10; C07D 403/04; C07D 471/04; C07D 249/18; C07D 235/26; C07D 491/107; C07D 413/10; C07D 403/10; C07D 487/08; C07D 401/14; C07D 401/12; C07D 417/10; C07D 495/10

(57) ABSTRACT

Described herein are compounds that are HSD17B13 inhibitors, methods of making such compounds, pharmaceutical compositions and medicaments comprising such compounds, and methods of using such compounds in the treatment of conditions, diseases, or disorders associated with HSD17B13 activity.

### CROSS-REFERENCE

This application claims benefit of U.S. Provisional Patent Application No. 63/085,843, filed on Sep. 30, 2020 which is incorporated herein by reference in its entirety.

### FIELD OF THE INVENTION

Described herein are compounds that are hydroxysteroid 17β-dehydrogenase 13 (HSD17B13) inhibitors, methods of making such compounds, pharmaceutical compositions and medicaments comprising such compounds, and methods of using such compounds in the treatment of conditions, diseases, or disorders associated with HSD17B13 activity.

### BACKGROUND OF THE INVENTION

Hydroxysteroid dehydrogenase 17013 (HSD17b13) is a member of the short-chain dehydrogenase/reductase enzymes highly expressed in the liver on lipid droplets. It has been shown to oxidize retinol, steroids such as estradiol, and bio-active lipids like leukotriene B4. Loss of HSD17b13 expression and enzymatic activity is associated with decreased incidence of liver disease. Inhibition of HSD17b13 enzymatic activity can be used for the treatment of liver diseases that result in hepatic inflammation, fibrosis, cirrhosis, and development of hepatocellular carcinoma.

### SUMMARY OF THE INVENTION

In one aspect, described herein is a compound of Formula (I), or a pharmaceutically acceptable salt or solvate thereof:

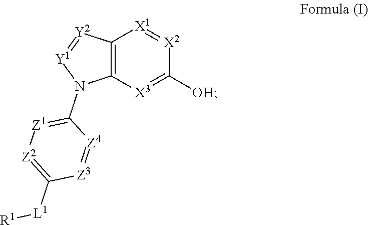

wherein:
   X¹, X², and X³ are each independently CR³ or N;
   Y¹ and Y² are each independently CR⁴ or N;
   Z¹ is CR⁵ or N;
   Z² is CR⁵ or N;
   Z³ is CR⁵ or N;
   Z⁴ is CR⁵;
   L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
   R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
   each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
   each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
   each R⁴ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹), —S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
   each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
   each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
   each R¹¹ is independently selected from hydrogen, C₁₋₆alkyl, and C₁₋₆haloalkyl;
   each R¹² is independently selected from hydrogen, C₁₋₆alkyl, and C₁₋₆haloalkyl; and
   each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (I), or a pharmaceutically acceptable salt or solvate thereof, wherein X¹, X², and X³ are CR³. In some embodiments is a compound of Formula (I), or a pharmaceutically acceptable salt or solvate thereof, wherein Y² is CR⁴.

In some embodiments is a compound of Formula (I), or a pharmaceutically acceptable salt or solvate thereof, having the structure of Formula (Ia):

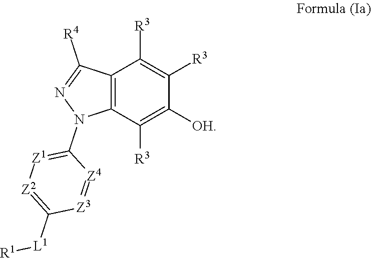

In some embodiments is a compound of Formula (I), or a pharmaceutically acceptable salt or solvate thereof, wherein Y² is N.

In some embodiments is a compound of Formula (I), or a pharmaceutically acceptable salt or solvate thereof, having the structure of Formula (Ib):

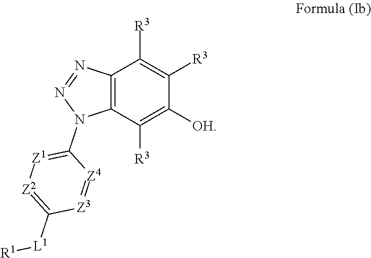

In one aspect, described herein is a compound of Formula (II), or a pharmaceutically acceptable salt or solvate thereof:

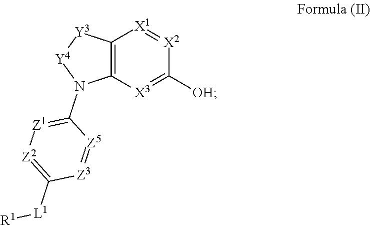

wherein:
   X¹, X², and X³ are each independently CR³ or N;
   Y³ is C(O), C(R⁴)₂, N(R⁶), O, or S;
   Y⁴ is C(O), C(R⁴)₂, or N(R⁶), wherein at least one of Y³ and Y⁴ is C(O);
   Z¹ is CR⁵ or N;
   Z² is CR⁵ or N;
   Z³ is CR⁵ or N;
   Z⁵ is CR⁵ or N;
   L¹ is selected from a bond, —O—, —S—, —N(R¹²)—, —C(O)—, —S(O)—, —S(O)₂—, C(R¹⁰)(R¹¹)N(R¹⁰)—, and —N(R¹²)C(R¹⁰)(R¹¹)—;
   R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
   each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
   each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
   each R⁴ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
   each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
   R⁶ is selected from hydrogen, C₁₋₆alkyl, C₁₋₆ haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —C(O)R¹³, —S(O)R¹³, —S(O)₂R¹³, —C(O)OR¹⁰, —S(O)₂OR¹⁰, —C(O)N(R¹⁰)(R¹¹), —S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, C₁₋₆alkyl, C₁₋₆haloalkyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —C(O)N(R¹⁰)(R¹¹), —S(O)R¹³, —S(O)₂R¹³, —S(O)₂N(R¹⁰)(R¹¹), —OC(O)N(R¹⁰)(R¹¹), —N(R¹²)C(O)R¹³, —N(R¹²)C(O)OR¹³, and —N(R¹²)C(O)N(R¹⁰)(R¹¹);
   each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
   each R¹¹ is independently selected from hydrogen, C₁₋₆alkyl, and C₁₋₆haloalkyl;
   each R¹² is independently selected from hydrogen, C₁₋₆alkyl, and C₁₋₆haloalkyl; and
   each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (II), or a pharmaceutically acceptable salt or solvate thereof, wherein X¹, X², and X³ are CR³.

In some embodiments is a compound of Formula (II), or a pharmaceutically acceptable salt or solvate thereof, having the structure of Formula (IIa):

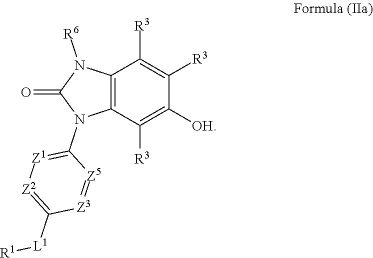

In some embodiments is a compound of Formula (II) or (IIa), or a pharmaceutically acceptable salt or solvate thereof, wherein R⁶ is selected from hydrogen and C₁₋₆alkyl optionally substituted with one, two, or three groups selected from halogen, —CN, C₁₋₆alkyl, C₁₋₆haloalkyl, —OR¹⁰, and —N(R¹⁰)(R¹¹). In some embodiments is a compound of Formula (II) or (IIa), or a pharmaceutically acceptable salt or solvate thereof, wherein R⁶ is C₁₋₆alkyl optionally substituted with —OH.

In some embodiments is a compound of Formula (II), or a pharmaceutically acceptable salt or solvate thereof, having the structure of Formula (IIb):

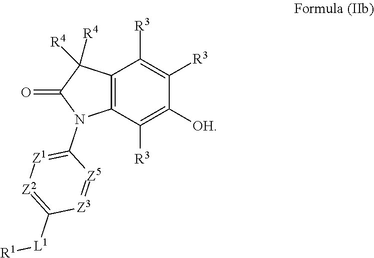

In some embodiments is a compound of Formula (II), (IIa), or (IIb), or a pharmaceutically acceptable salt or solvate thereof, wherein Z⁵ is CR⁵. In some embodiments is a compound of Formula (II), (IIa), or (IIb), or a pharmaceutically acceptable salt or solvate thereof, wherein Z⁵ is N.

In some embodiments is a compound of Formula (I), (Ia), (Ib), (II), (IIa), or (IIb), or a pharmaceutically acceptable salt or solvate thereof, wherein Z¹, Z², and Z³ are CR⁵. In some embodiments is a compound of Formula (I), (Ia), (Ib), (II), (IIa), or (IIb), or a pharmaceutically acceptable salt or solvate thereof, wherein Z¹ is N; and Z² and Z³ are CR⁵. In some embodiments is a compound of Formula (I), (Ia), (Ib), (II), (IIa), or (IIb), or a pharmaceutically acceptable salt or solvate thereof, wherein Z³ is N; and Z¹ and Z² are CR⁵. In some embodiments is a compound of Formula (I), (Ia), (Ib), (II), (IIa), or (IIb), or a pharmaceutically acceptable salt or solvate thereof, wherein Z¹ is CR⁵; and Z² and Z³ are N. In some embodiments is a compound of Formula (I), (Ia), (Ib), (II), (IIa), or (IIb), or a pharmaceutically acceptable salt or solvate thereof, wherein Z² is CR⁵; and Z¹ and Z³ are N. In some embodiments is a compound of Formula (I), (Ia), (Ib), (II), (IIa), or (IIb), or a pharmaceutically acceptable salt or solvate thereof, wherein Z³ is CR⁵; and Z¹ and Z² are N. In some embodiments is a compound of Formula (I), (Ia), (Ib), (II), (IIa), or (IIb), or a pharmaceutically acceptable salt or solvate thereof, wherein L¹ is a bond. In some embodiments is a compound of Formula (I), (Ia), (Ib), (II), (IIa), or (IIb), or a pharmaceutically acceptable salt or solvate thereof, wherein R¹ is C₂₋₉heterocycloalkyl optionally substituted with one, two, three, four, or five R². In some embodiments is a compound of Formula (I), (Ia), (Ib), (II), (IIa), or (IIb), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, and aziridinyl, wherein piperidinyl, piperazinyl, morpholinyl, tetrahydropyranyl, tetrahydrofuranyl, pyrrolidinyl, oxetanyl, azetidinyl, aziridinyl, azepanyl, and diazepanyl are optionally substituted with one, two, or three R². In some embodiments is a compound of Formula (I), (Ia), (Ib), (II), (IIa), or (IIb), or a pharmaceutically acceptable salt or solvate thereof, wherein R¹ is C₂₋₉heterocycloalkyl selected from thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane,2,6-diazaspiro[3.3]heptane, and 2-oxa-6-azaspiro[3.3]heptane, wherein thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane, 2,6-diazaspiro[3.3]heptane, and 2-oxa-6-azaspiro[3.3]heptane are optionally substituted with one, two, or three R². In some embodiments is a compound of Formula (I), (Ia), (Ib), (II), (IIa), or (IIb), or a pharmaceutically acceptable salt or solvate thereof, wherein R¹ is C₂₋₉heterocycloalkyl selected from thiomorpholine, 2-oxa-7-azaspiro[3.5]nonane, 7-oxa-2-azaspiro[3.5]nonane, 2,7-diazaspiro[3.5]nonane, 2,6-diazaspiro[3.3]heptane, wherein thiomorpholine, 2-oxa-7-azaspiro[3.5]nonane, 7-oxa-2-azaspiro[3.5]nonane, 2,7-diazaspiro[3.5]nonane, and 2,6-diazaspiro[3.3]heptane are optionally substituted with one, two, or three R². In some embodiments is a compound of Formula (I), (Ia), (Ib), (II), (IIa), or (IIb), or a pharmaceutically acceptable salt or solvate thereof, wherein R¹ is

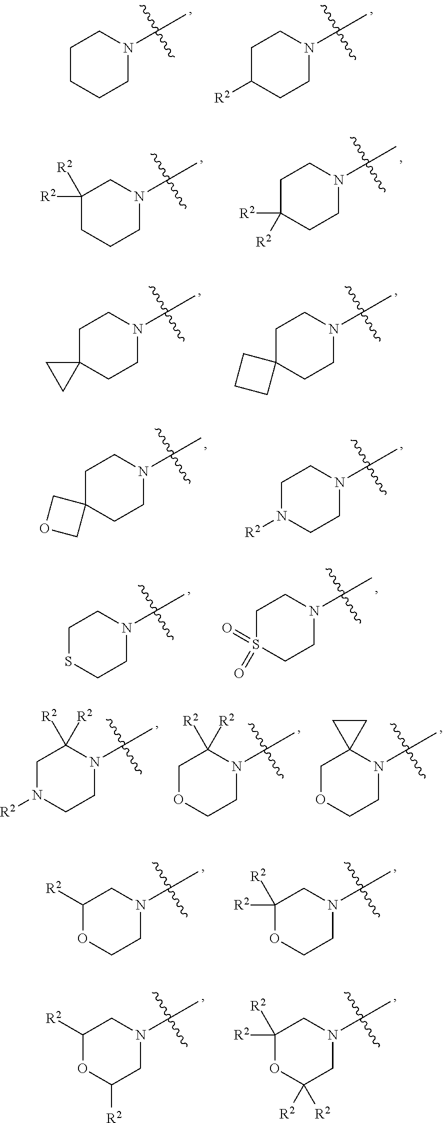

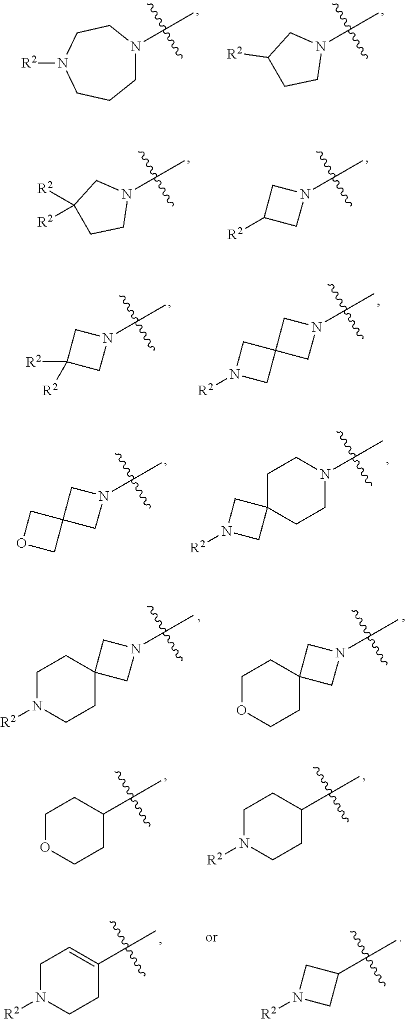

In some embodiments is a compound of Formula (I), (Ia), (Ib), (II), (IIa), or (IIb), or a pharmaceutically acceptable salt or solvate thereof, wherein each R² is independently selected from C₁₋₆alkyl, C₁₋₆ haloalkyl, —CN, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—.

In some embodiments is a compound of Formula (I), (Ia), (Ib), (II), (IIa), or (IIb), or a pharmaceutically acceptable salt or solvate thereof, wherein R¹ is

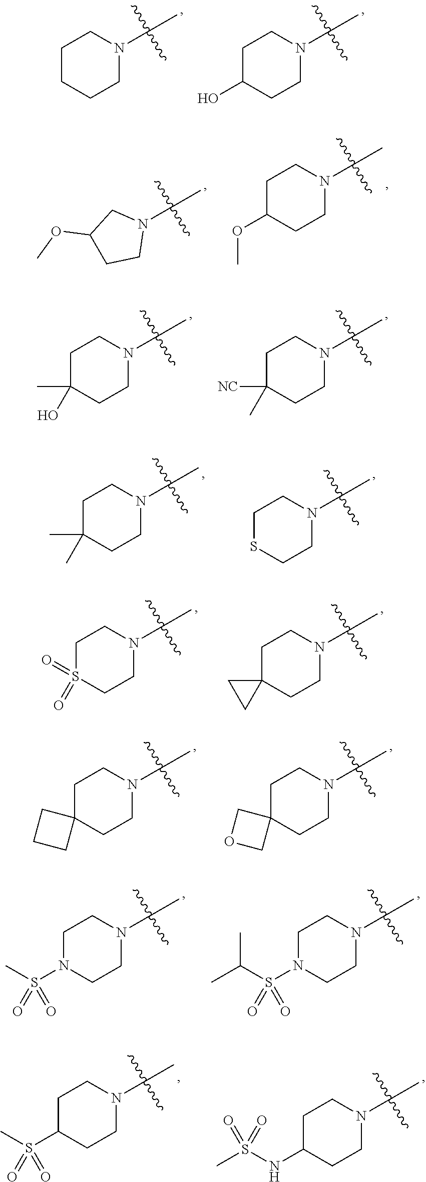

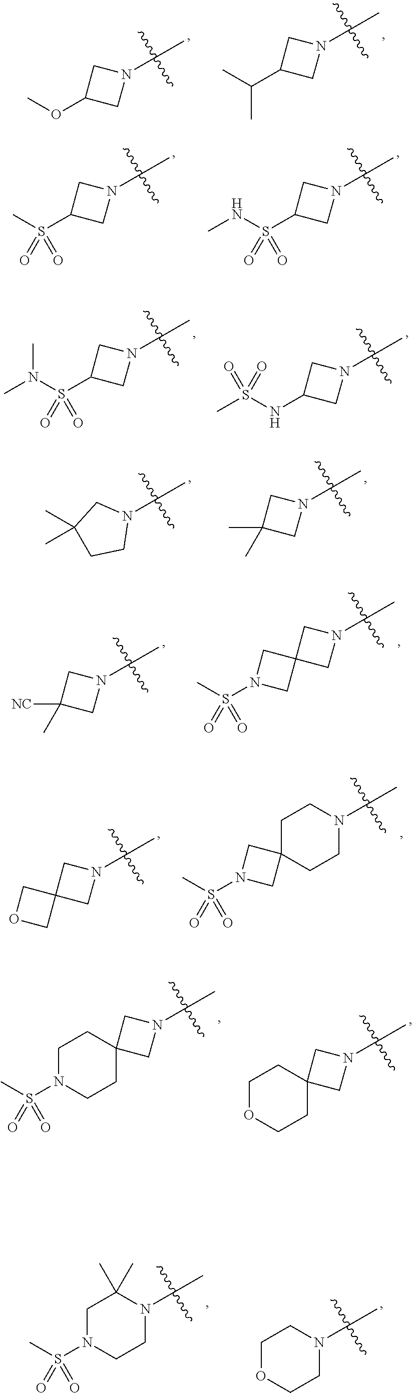

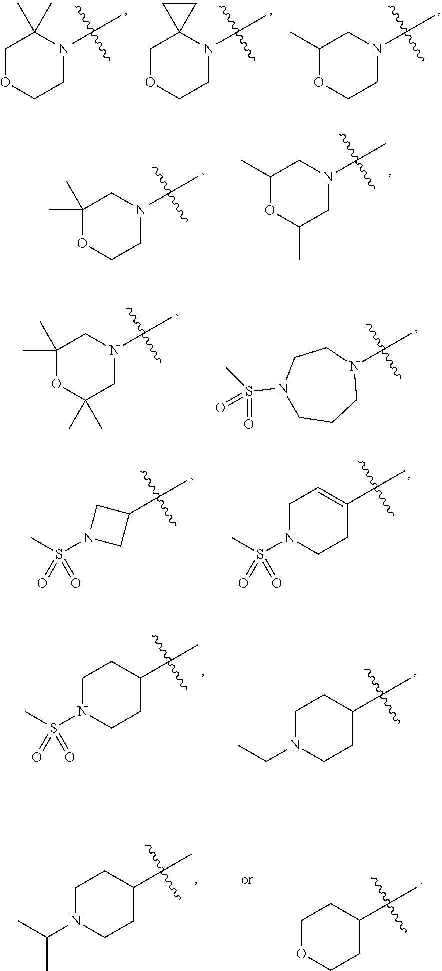

In some embodiments is a compound of Formula (I), (Ia), (Ib), (II), (IIa), or (IIb), or a pharmaceutically acceptable salt or solvate thereof, wherein R¹ is selected from C₆₋₁₀aryl and C₁₋₉heteroaryl, wherein C₆₋₁₀aryl and C₁₋₉ heteroaryl are substituted with one, two, or three R². In some embodiments is a compound of Formula (I), (Ia), (Ib), (II), (IIa), or (IIb), or a pharmaceutically acceptable salt or solvate thereof, wherein R¹ is phenyl substituted with one, two, or three R². In some embodiments is a compound of Formula (I), (Ia), (Ib), (II), (IIa), or (IIb), or a pharmaceutically acceptable salt or solvate thereof, wherein R¹ is C₁₋₉heteroaryl substituted with one, two, or three R². In some embodiments is a compound of Formula (I), (Ia), (Ib), (II), (IIa), or (IIb), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R². In some embodiments is a compound of Formula (I), (Ia), (Ib), (II), (IIa), or (IIb), or a pharmaceutically acceptable salt or solvate thereof, wherein each R² is independently selected from halogen, —CN, C₁₋₆alkyl, C₁₋₆ haloalkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹). In some embodiments is a compound of Formula (I), (Ia), (Ib), (II), (IIa), or (IIb), or a pharmaceutically acceptable salt or solvate thereof, wherein each R² is independently selected from halogen, —CN, C₁₋₆alkyl, —OH, —N(H)S(O)₂CH₃, —S(O)₂CH₃, and —S(O)₂NH₂. In some embodiments is a compound of Formula (I), (Ia), (Ib), (II), (IIa), or (IIb), or a pharmaceutically acceptable salt or solvate thereof, wherein each R⁵ is independently selected from hydrogen, halogen, —CN, C₁₋₆alkyl, C₁₋₆ haloalkyl, and —OR¹⁰. In some embodiments is a compound of Formula (I), (Ia), (Ib), (II), (IIa), or (IIb), or a pharmaceutically acceptable salt or solvate thereof, wherein each R⁵ is hydrogen. In some embodiments is a compound of Formula (I), (Ia), (Ib), (II), (IIa), or (IIb), or a pharmaceutically acceptable salt or solvate thereof, wherein each R⁵ is H. In some embodiments is a compound of Formula (I), (Ia), (Ib), (II), (IIa), or (IIb), or a pharmaceutically acceptable salt or solvate thereof, wherein each R⁴ is independently selected from hydrogen, halogen, —CN, C₁₋₆alkyl, C₁₋₆ haloalkyl, and C₃₋₆ cycloalkyl. In some embodiments is a compound of Formula (I), (Ia), (Ib), (II), (IIa), or (IIb), or a pharmaceutically acceptable salt or solvate thereof, wherein each R⁴ is hydrogen. In some embodiments is a compound of Formula (I), (Ia), (Ib), (II), (IIa), or (IIb), or a pharmaceutically acceptable salt or solvate thereof, wherein each R³ is independently selected from hydrogen, halogen, —CN, C₁₋₆alkyl, C₁₋₆haloalkyl, and —OH. In some embodiments is a compound of Formula (I), (Ia), (Ib), (II), (IIa), or (IIb), or a pharmaceutically acceptable salt or solvate thereof, wherein each R³ is independently selected from hydrogen, halogen, —CN, and CF₃.

Any combination of the groups described above for the various variables is contemplated herein. Throughout the specification, groups and substituents thereof are chosen by one skilled in the field to provide stable moieties and compounds.

In one aspect, described herein is a pharmaceutical composition comprising a compound described herein, or a pharmaceutically acceptable salt or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In another aspect, described herein is a method of treating or preventing a liver disease or condition in a mammal, comprising administering to the mammal a compound of Formula (I), (Ia), (Ib), (II), (IIa), or (IIb), or a pharmaceutically acceptable salt or solvate thereof. In some embodiments, the liver disease or condition is an alcoholic liver disease or condition. In some embodiments, the liver disease or condition is a nonalcoholic liver disease or condition. In some embodiments, the liver disease or condition is liver inflammation, fatty liver (steatosis), liver fibrosis, hepatitis, cirrhosis, hepatocellular carcinoma, or combinations thereof. In some embodiments, the liver disease or condition is primary biliary cirrhosis, primary sclerosing cholangitis, cholestasis, nonalcoholic steatohepatitis (NASH), nonalcoholic fatty liver disease (NAFLD), or combinations thereof.

In another aspect, described herein is a method of treating a disease or condition in a mammal that would benefit from hydroxysteroid 17β-dehydrogenase 13 (HSD17B13) inhibition comprising administering a compound as described herein, or pharmaceutically acceptable salt or solvate thereof, to the mammal in need thereof. In some embodiments, the disease or condition in a mammal that would benefit from HSD17B13 inhibition is liver inflammation, fatty liver (steatosis), liver fibrosis, hepatitis, cirrhosis, hepatocellular carcinoma, or combinations thereof. In some embodiments, the disease or condition in a mammal that would benefit from HSD17B13 inhibition is primary biliary cirrhosis, primary sclerosing cholangitis, cholestasis, nonalcoholic steatohepatitis (NASH), nonalcoholic fatty liver disease (NAFLD), or combinations thereof.

In another aspect, described herein is a method of modulating hydroxysteroid 17β-dehydrogenase 13 (HSD17B13) activity in a mammal, comprising administering to the mammal a compound of Formula (I), (Ia), (Ib), (II), (IIa), or (IIb), or a pharmaceutically acceptable salt or solvate thereof. In some embodiments, modulating comprises inhibiting HSD17B13 activity. In some embodiments of a method of modulating HSD17B13 activity in a mammal, the mammal has a liver disease or condition selected from liver inflammation, fatty liver (steatosis), liver fibrosis, hepatitis, cirrhosis, hepatocellular carcinoma, and combinations thereof. In some embodiments of a method of modulating HSD17B13 activity in a mammal, the mammal has a liver disease or condition selected from primary biliary cirrhosis, primary sclerosing cholangitis, cholestasis, nonalcoholic steatohepatitis (NASH), nonalcoholic fatty liver disease (NAFLD), and combinations thereof.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the embodiments disclosed herein, the mammal or subject is a human.

In some embodiments, compounds provided herein are administered to a human.

In some embodiments, compounds provided herein are orally administered.

Articles of manufacture, which include packaging material, a compound described herein, or a pharmaceutically acceptable salt thereof, within the packaging material, and a label that indicates that the compound or composition, or pharmaceutically acceptable salt, pharmaceutically active metabolite, pharmaceutically acceptable prodrug, or pharmaceutically acceptable solvate thereof, is used for the treatment, prevention or amelioration of one or more symptoms of a disease or condition that would benefit from HSD17B13 inhibition, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Hydroxysteroid dehydrogenase 17313 (HSD17b13) is a member of the short-chain dehydrogenase/reductase enzymes highly expressed in the liver on lipid droplets (Horiguchi et al Biochem Biophysl Res Comm, 2008, 370, 235). It has been shown to oxidize retinol, steroids such as estradiol, and bio-active lipids like leukotriene B4 (Abul-Husn et al NEJM, 2018, 378, 1096 and Ma et al Hepatology, 2019, 69 1504). Exosome sequencing analysis of a large patient population identified a minor allele of HSD17b13 (rs72613567:TA) that was associated with reduced odds of developing liver disease (Abul-Husn et al NEJM, 2018, 378, 1096). Relative to subjects with the common HSD17b13 allele (rs72613567:T), subjects with the TA variant have lower serum ALT and AST and lower odds of alcoholic liver disease with or without cirrhosis, nonalcoholic liver disease with or without cirrhosis, and lower odds of hepatocellular carcinoma. Liver pathology analysis reveals that the subjects with the rs72613567:TA allele have decreased odds of having liver pathology analysis classified as NASH vs normal, NASH vs simple steatosis or NASH with fibrosis vs simple steatosis. Liver injury associated with the PNPLA3 rs738409 (p.I148M) is mitigated by the presence of the rs72613567:TA allele of HSD17b13. Additionally hepatic PNPLA3 mRNA expression is decreased in subjects with the rs72613567:TA allele. The rs72613567:TA allele was found to produce a truncated protein which is unable to metabolize substrates such as estradiol, suggesting the hepatic protective effects of the rs72613567:TA allele is due to loss of enzymatic activity.

Patients with NASH have shown elevated expression of hepatic of HSD17b13 mRNA relative to control subject. Further exploration of the role of HSD17b13 in NASH development identified a minor allele rs62305723 that encodes a P260S mutation of HSD17b13 that leads to loss of retinol metabolism and is associated with decreased hepatic ballooning and inflammation (Ma et al Hepatology, 2019, 69 1504).

HSD17b13 rs72613567:TA minor allele is associated with loss of HSD17b13 protein expression in the liver and protection from nonalcoholic steatohepatitis, ballooning degeneration, lobular inflammation and fibrosis. Transcription analysis shows changes in immune-responsive pathways in subjects with rs72613567:TA relative to the major allele (Pirolat et al JLR, 2019, 60, 176).

Subjects with the rs72613567:TA allele of HSD17b13 are not only found to have lower histological evidence of fibrosis, but decreased hepatic expression of fibrotic genes like TGFb2 and Col3a1. In addition loss of HSD17b13 due to the rs72613567:TA allele has been shown to significantly change the expression of inflammatory gene ALOX5 and decreased plasma IL1b, IL6 and IL-10 (Luukkonen et al, JCI, 2020, 5 e132158). HSD17b13 rs72613567:TA carriers also show increased hepatic phospholipids PC(p16:0/16:0), PE(p16:0/18:1), PC(44:5e), PC(36:2e), PE(34:0), PE(36:3) and PC(34:3) possibly due to decreased phospholipid degradation from a decreased hepatic expression of PLD4.

The HSD17b13 rs72613567:TA allele, that has been shown to lack HSD17b13 enzymatic activity, is associated with decreased odds of developing severe fibrosis in patients with chronic HCV infection (About& Abel, NEJM, 2018, 379, 1875). Conversely the major allele rs72613567:T is associated with increasing the risk of development of fibrosis, cirrhosis and HCC in HCV infected patients with the PNPLA3 rs738409:G allele (De Benedittis et al. Gastroenterol Res Pract, 2020, 2020, 4216451).

The loss of function minor allele HSD17b13 rs72613567:TA reduces the risk of developing cirrhosis and hepatocellular carcinoma, is associated with a lower risk of liver-related mortality in the general population and further in patients with cirrhosis (Gellbert-Kristensen et al, Hepatology, 2020, 71, 56). Loss of HSD17b13 function also protects against development of HCC in subjects with alcoholic liver disease (Yang et al, Hepatology, 2019, 70, 231 and Stickel et al, Hepatology, 2020, 72, 88).

PNPLA3 rs738409:G is associated with increased fibrosis in patients with NAFLD. The minor HSD17b13 rs72613567:TA allele has been shown to contact the PNPLA3 rs738409:G allele and decrease the prevalence of severe inflammation, ballooning and fibrosis (Seko et al, Liver Int, 2020, 40, 1686).

Loss of HSD17b13 enzymatic activity due to carrying the rs72613567:TA allele may delay the onset of autoimmune hepatitis (Mederacke et al, Aliment Pharmacol Ther, 2020, 00, 1).

HSD17b13 rs72613567:TA allele is associated with decreased fibrosis and cirrhosis in patents with copper induced liver injury from Wilson's disease (Ferenci et al, 2019, JHEP, 1, 2).

### Compounds

Compounds described herein, including pharmaceutically acceptable salts, prodrugs, active metabolites and pharmaceutically acceptable solvates thereof, are HSD17B13 inhibitors.

In some embodiments is a compound of Formula (I), or a pharmaceutically acceptable salt or solvate thereof:

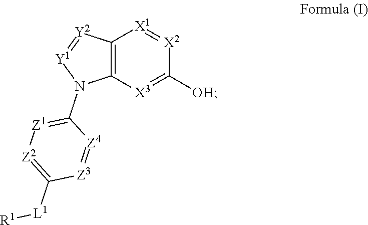

wherein:
X¹, X², and X³ are each independently CR³ or N;
Y¹ and Y² are each independently CR⁴ or N;
Z¹ is CR⁵ or N;
Z² is CR⁵ or N;
Z³ is CR⁵ or N;
Z⁴ is CR⁵;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁴ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹), —S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (I), or a pharmaceutically acceptable salt or solvate thereof, wherein X¹, X², and X³ are each CR³. In some embodiments is a compound of Formula (I), or a pharmaceutically acceptable salt or solvate thereof, wherein X¹, X², and X³ are each CR³ and each R³ is independently selected from hydrogen, halogen, C₁₋₆alkyl, C₁₋₆haloalkyl, and —OR¹⁰. In some embodiments is a compound of Formula (I), or a pharmaceutically acceptable salt or solvate thereof, wherein X¹, X², and X³ are each CR³ and each R³ is independently selected from hydrogen, halogen, C₁₋₆alkyl, and C₁₋₆haloalkyl. In some embodiments is a compound of Formula (I), or a pharmaceutically acceptable salt or solvate thereof, wherein X¹, X², and X³ are each CR³ and each R³ is independently selected from hydrogen, halogen, and C₁₋₆haloalkyl. In some embodiments is a compound of Formula (I), or a pharmaceutically acceptable salt or solvate thereof, wherein X¹, X², and X³ are each CR³ and each R³ is independently selected from hydrogen, halogen, —CN, C₁₋₆alkyl, C₁₋₆haloalkyl, and —OH. In some embodiments is a compound of Formula (I), or a pharmaceutically acceptable salt or solvate thereof, wherein X¹, X², and X³ are each CR³ and each R³ is independently selected from hydrogen, halogen, —CN, and CF₃. In some embodiments is a compound of Formula (I), or a pharmaceutically acceptable salt or solvate thereof, wherein X¹ is C(H), X² is C(H), and X³ is C(CF₃). In some embodiments is a compound of Formula (I), or a pharmaceutically acceptable salt or solvate thereof, wherein X¹ is C(F), X² is C(H), and X³ is C(CF₃). In some embodiments is a compound of Formula (I), or a pharmaceutically acceptable salt or solvate thereof, wherein X¹ is C(CF₃), X² is C(F), and X³ is C(H). In some embodiments is a compound of Formula (I), or a pharmaceutically acceptable salt or solvate thereof, wherein X¹ is C(H), X² is C(F), and X³ is C(CF₃). In some embodiments is a compound of Formula (I), or a pharmaceutically acceptable salt or solvate thereof, wherein X¹ is C(Cl), X² is C(H), and X³ is C(CF₃). In some embodiments is a compound of Formula (I), or a pharmaceutically acceptable salt or solvate thereof, wherein X¹ is C(H), X² is C(F), and X³ is C(CN). In some embodiments is a compound of Formula (I), or a pharmaceutically acceptable salt or solvate thereof, wherein X¹ is C(H), X² is C(H), and X³ is C(F). In some embodiments is a compound of Formula (I), or a pharmaceutically acceptable salt or solvate thereof, wherein X¹ is C(H), X² is C(F), and X³ is C(H). In some embodiments is a compound of Formula (I), or a pharmaceutically acceptable salt or solvate thereof, wherein X¹ is C(F), X² is C(H), and X³ is C(H). In some embodiments is a compound of Formula (I), or a pharmaceutically acceptable salt or solvate thereof, wherein X¹ is C(H), X² is C(F), and X³ is C(F). In some embodiments is a compound of Formula (I), or a pharmaceutically acceptable salt or solvate thereof, wherein X¹ is C(F), X² is C(H), and X³ is C(F). In some embodiments is a compound of Formula (I), or a pharmaceutically acceptable salt or solvate thereof, wherein X¹ is C(F), X² is C(F), and X³ is C(H). In some embodiments is a compound of Formula (I), or a pharmaceutically acceptable salt or solvate thereof, wherein X¹ is C(H), X² is C(H), and X³ is C(Cl). In some embodiments is a compound of Formula (I), or a pharmaceutically acceptable salt or solvate thereof, wherein X¹ is C(Cl), X² is C(F), and X³ is C(H). In some embodiments is a compound of Formula (I), or a pharmaceutically acceptable salt or solvate thereof, wherein X¹ is C(H), X² is C(F), and X³ is C(CH₃).

In some embodiments is a compound of Formula (I), or a pharmaceutically acceptable salt or solvate thereof, wherein Y¹ is N. In some embodiments is a compound of Formula (I), or a pharmaceutically acceptable salt or solvate thereof, wherein Y¹ is CR⁴. In some embodiments is a compound of Formula (I), or a pharmaceutically acceptable salt or solvate thereof, wherein Y¹ is CR⁴ and R⁴ is selected from hydrogen, halogen, —CN, C₁₋₆alkyl, C₁₋₆ haloalkyl, and C₃₋₆cycloalkyl. In some embodiments is a compound of Formula (I), or a pharmaceutically acceptable salt or solvate thereof, wherein Y¹ is CR⁴ and R⁴ is hydrogen.

In some embodiments is a compound of Formula (I), or a pharmaceutically acceptable salt or solvate thereof, wherein Y² is CR⁴. In some embodiments is a compound of Formula (I), or a pharmaceutically acceptable salt or solvate thereof, wherein Y² is CR⁴ and R⁴ is selected from hydrogen, halogen, —CN, C₁₋₆alkyl, C₁₋₆ haloalkyl, and C₃₋₆cycloalkyl. In some embodiments is a compound of Formula (I), or a pharmaceutically acceptable salt or solvate thereof, wherein Y² is CR⁴ and R⁴ is hydrogen. In some embodiments is a compound of Formula (I), or a pharmaceutically acceptable salt or solvate thereof, wherein Y² is N.

In some embodiments is a compound of Formula (I), or a pharmaceutically acceptable salt or solvate thereof, wherein Y¹ is N and Y² is CR⁴. In some embodiments is a compound of Formula (I), or a pharmaceutically acceptable salt or solvate thereof, wherein Y¹ is CR⁴ and Y² is CR⁴. In some embodiments is a compound of Formula (I), or a pharmaceutically acceptable salt or solvate thereof, wherein Y¹ is CR⁴ and Y² is N. In some embodiments is a compound of Formula (I), or a pharmaceutically acceptable salt or solvate thereof, wherein each R⁴ is independently selected from hydrogen, halogen, C₁₋₆alkyl, and C₃₋₆ cycloalkyl. In some embodiments is a compound of Formula (I), or a pharmaceutically acceptable salt or solvate thereof, wherein Y¹ is N and Y² is C(H). In some embodiments is a compound of Formula (I), or a pharmaceutically acceptable salt or solvate thereof, wherein Y¹ is C(H) and Y² is C(H). In some embodiments is a compound of Formula (I), or a pharmaceutically acceptable salt or solvate thereof, wherein Y¹ is C(H) and Y² is N. In some embodiments is a compound of Formula (I), or a pharmaceutically acceptable salt or solvate thereof, wherein Y¹ is N and Y² is N.

In some embodiments is a compound of Formula (I), or a pharmaceutically acceptable salt or solvate thereof, wherein Z¹, Z², and Z³ are CR⁵. In some embodiments is a compound of Formula (I), or a pharmaceutically acceptable salt or solvate thereof, wherein Z¹ is N; and Z² and Z³ are CR⁵. In some embodiments is a compound of Formula (I), or a pharmaceutically acceptable salt or solvate thereof, wherein Z² is N; and Z¹ and Z³ are CR⁵. In some embodiments is a compound of Formula (I), or a pharmaceutically acceptable salt or solvate thereof, wherein Z³ is N; and Z¹ and Z² are CR⁵. In some embodiments is a compound of Formula (I), or a pharmaceutically acceptable salt or solvate thereof, wherein Z¹ is CR⁵; and Z² and Z³ are N. In some embodiments is a compound of Formula (I), or a pharmaceutically acceptable salt or solvate thereof, wherein Z² is CR⁵; and Z¹ and Z³ are N. In some embodiments is a compound of Formula (I), or a pharmaceutically acceptable salt or solvate thereof, wherein Z³ is CR⁵; and Z¹ and Z² are N. In some embodiments is a compound of Formula (I), or a pharmaceutically acceptable salt or solvate thereof, wherein each R⁵ is independently selected from hydrogen, halogen, C₁₋₆alkyl, and —OR¹⁰. In some embodiments is a compound of Formula (I), or a pharmaceutically acceptable salt or solvate thereof, wherein each R⁵ is H. In some embodiments is a compound of Formula (I), or a pharmaceutically acceptable salt or solvate thereof, wherein Z¹, Z², and Z³ are C(H). In some embodiments is a compound of Formula (I), or a pharmaceutically acceptable salt or solvate thereof, wherein Z¹ is N; and Z² and Z³ are C(H). In some embodiments is a compound of Formula (I), or a pharmaceutically acceptable salt or solvate thereof, wherein Z² is N; and Z¹ and Z³ are C(H). In some embodiments is a compound of Formula (I), or a pharmaceutically acceptable salt or solvate thereof, wherein Z³ is N; and Z¹ and Z² are C(H). In some embodiments is a compound of Formula (I), or a pharmaceutically acceptable salt or solvate thereof, wherein Z¹ is C(H); and Z² and Z³ are N. In some embodiments is a compound of Formula (I), or a pharmaceutically acceptable salt or solvate thereof, wherein Z² is C(H); and Z¹ and Z³ are N. In some embodiments is a compound of Formula (I), or a pharmaceutically acceptable salt or solvate thereof, wherein Z³ is C(H); and Z¹ and Z² are N.

In some embodiments is a compound of Formula (I), or a pharmaceutically acceptable salt or solvate thereof, wherein Z⁴ is C(H).

In some embodiments is a compound of Formula (I), or a pharmaceutically acceptable salt or solvate thereof, wherein L¹ is a bond. In some embodiments is a compound of Formula (I), or a pharmaceutically acceptable salt or solvate thereof, wherein L¹ is —O—. In some embodiments is a compound of Formula (I), or a pharmaceutically acceptable salt or solvate thereof, wherein L¹ is —N(R¹⁰)—. In some embodiments is a compound of Formula (I), or a pharmaceutically acceptable salt or solvate thereof, wherein L¹ is —N(H)—. In some embodiments is a compound of Formula (I), or a pharmaceutically acceptable salt or solvate thereof, wherein L¹ is —C(R¹⁰)(R¹¹)N(R¹⁰)—. In some embodiments is a compound of Formula (I), or a pharmaceutically acceptable salt or solvate thereof, wherein L¹ is —CH₂N(H)—. In some embodiments is a compound of Formula (I), or a pharmaceutically acceptable salt or solvate thereof, wherein L¹ is —N(R¹⁰)C(R¹⁰)(R¹¹)—. In some embodiments is a compound of Formula (I), or a pharmaceutically acceptable salt or solvate thereof, wherein L¹ is —N(H)CH₂—.

In some embodiments is a compound of Formula (I), or a pharmaceutically acceptable salt or solvate thereof, wherein R¹ is C₂₋₉heterocycloalkyl optionally substituted with one, two, three, four, or five R². In some embodiments is a compound of Formula (I), or a pharmaceutically acceptable salt or solvate thereof, wherein R¹ is C₂₋₉heterocycloalkyl optionally substituted with one, two, or three R². In some embodiments is a compound of Formula (I), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, and aziridinyl, wherein piperidinyl, piperazinyl, morpholinyl, tetrahydropyranyl, tetrahydrofuranyl, pyrrolidinyl, oxetanyl, azetidinyl, aziridinyl, azepanyl, and diazepanyl are optionally substituted with one, two, or three R². In some embodiments is a compound of Formula (I), or a pharmaceutically acceptable salt or solvate thereof, wherein R¹ is C₂₋₉heterocycloalkyl selected from thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane, 2,6-diazaspiro[3.3]heptane, and 2-oxa-6-azaspiro[3.3]heptane, wherein thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane, 2,6-diazaspiro[3.3]heptane, and 2-oxa-6-azaspiro[3.3]heptane are optionally substituted with one, two, or three R². In some embodiments is a compound of Formula (I), or a pharmaceutically acceptable salt or solvate thereof, wherein R¹ is C₂₋₉heterocycloalkyl selected from thiomorpholine, 2-oxa-7-azaspiro[3.5]nonane, 7-oxa-2-azaspiro[3.5]nonane, 2,7-diazaspiro[3.5]nonane, 2,6-diazaspiro[3.3]heptane, wherein thiomorpholine, 2-oxa-7-azaspiro[3.5]nonane, 7-oxa-2-azaspiro[3.5]nonane, 2,7-diazaspiro[3.5]nonane, and 2,6-diazaspiro[3.3]heptane are optionally substituted with one, two, or three R². In some embodiments is a compound of Formula (I), or a pharmaceutically acceptable salt or solvate thereof, wherein R¹ is

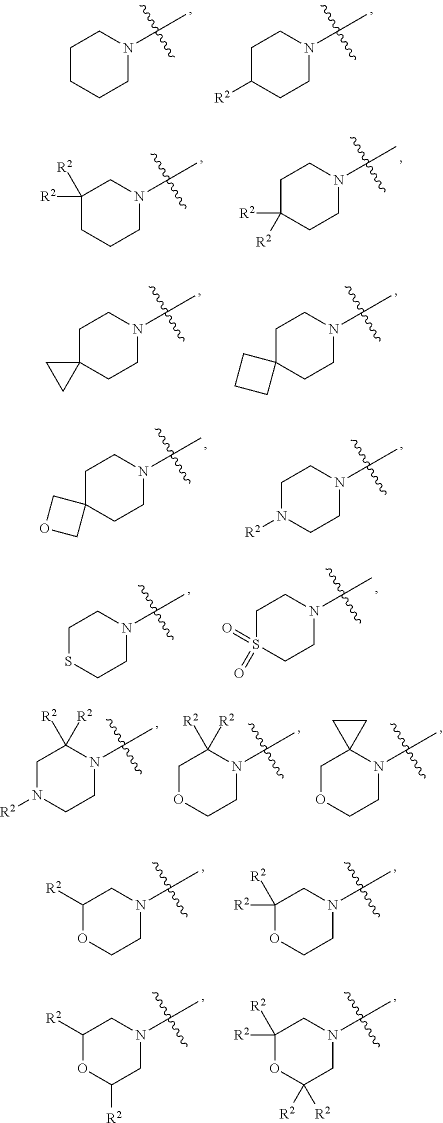

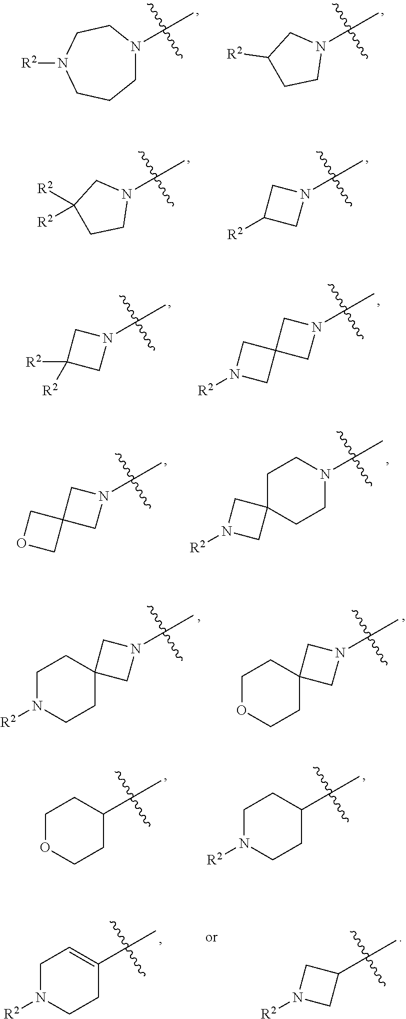

In some embodiments is a compound of Formula (I), or a pharmaceutically acceptable salt or solvate thereof, wherein each R² is independently selected from C₁₋₆alkyl, C₁₋₆ haloalkyl, —CN, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (I), or a pharmaceutically acceptable salt or solvate thereof, wherein R¹ is

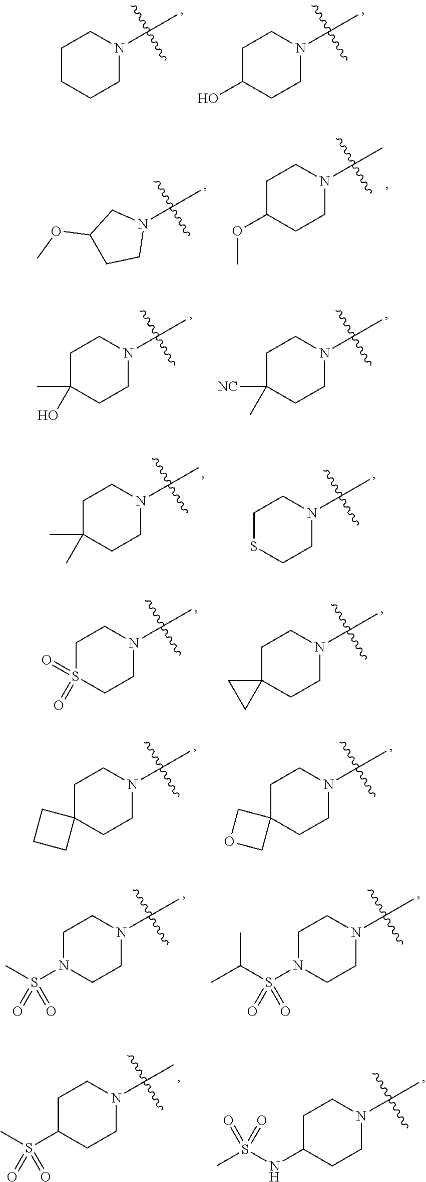

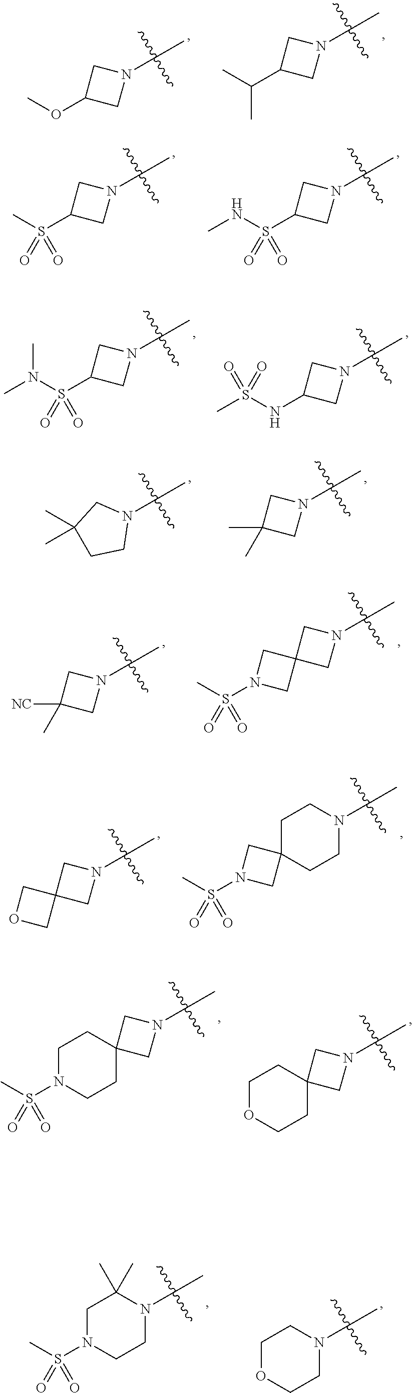

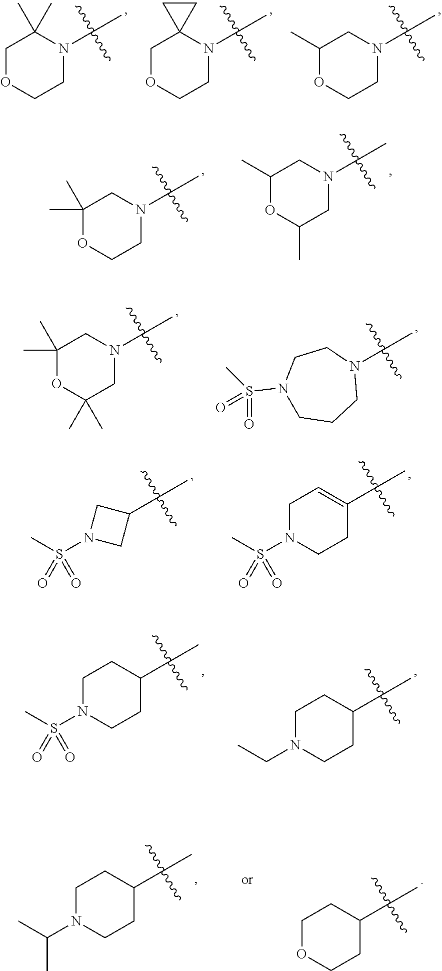

In some embodiments is a compound of Formula (I), or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R². In some embodiments is a compound of Formula (I), or a pharmaceutically acceptable salt or solvate thereof, wherein R¹ is phenyl substituted with one, two, or three R². In some embodiments is a compound of Formula (I), or a pharmaceutically acceptable salt or solvate thereof, wherein R¹ is C₁₋₉heteroaryl substituted with one, two, or three R². In some embodiments is a compound of Formula (I), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R².

In some embodiments is a compound of Formula (I), or a pharmaceutically acceptable salt or solvate thereof, wherein each R² is independently selected from halogen, —CN, C₁₋₆alkyl, C₁₋₆ haloalkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹). In some embodiments is a compound of Formula (I), or a pharmaceutically acceptable salt or solvate thereof, wherein each R² is independently selected from halogen, —CN, C₁₋₆alkyl, —OH, —N(H)S(O)₂CH₃, —S(O)₂CH₃, and —S(O)₂NH₂.

In some embodiments is a compound of Formula (Ia), or a pharmaceutically acceptable salt or solvate thereof:

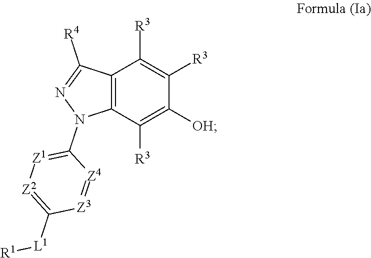

wherein:
Z¹ is CR⁵ or N;
Z² is CR⁵ or N;
Z³ is CR⁵ or N;
Z⁴ is CR⁵;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
R⁴ is selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹), —S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (Ia), or a pharmaceutically acceptable salt or solvate thereof, wherein R⁴ is selected from hydrogen, halogen, —CN, C₁₋₆ alkyl, C₁₋₆ haloalkyl, and C₃₋₆cycloalkyl. In some embodiments is a compound of Formula (Ia), or a pharmaceutically acceptable salt or solvate thereof, wherein R⁴ is hydrogen. In some embodiments is a compound of Formula (Ia), or a pharmaceutically acceptable salt or solvate thereof, wherein In some embodiments is a compound of Formula (Ia), or a pharmaceutically acceptable salt or solvate thereof, wherein R⁴ is halogen. In some embodiments is a compound of Formula (Ia), or a pharmaceutically acceptable salt or solvate thereof, wherein In some embodiments is a compound of Formula (Ia), or a pharmaceutically acceptable salt or solvate thereof, wherein R⁴ is —CN. In some embodiments is a compound of Formula (Ia), or a pharmaceutically acceptable salt or solvate thereof, wherein R⁴ is C₁₋₆ alkyl. In some embodiments is a compound of Formula (Ia), or a pharmaceutically acceptable salt or solvate thereof, wherein R⁴ is C₁₋₆ haloalkyl. In some embodiments is a compound of Formula (Ia), or a pharmaceutically acceptable salt or solvate thereof, wherein R⁴ is C₃₋₆cycloalkyl.

In some embodiments is a compound of Formula (Ib), or a pharmaceutically acceptable salt or solvate thereof:

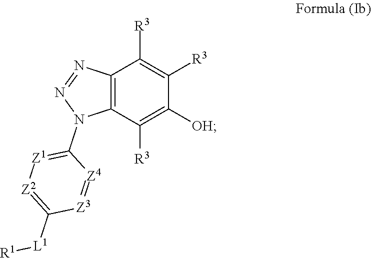

wherein:
Z¹ is CR⁵ or N;
Z² is CR⁵ or N;
Z³ is CR⁵ or N;
Z⁴ is CR⁵;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₈alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (Ia) or (Ib), or a pharmaceutically acceptable salt or solvate thereof, wherein Z¹, Z², and Z³ are CR⁵. In some embodiments is a compound of Formula (Ia) or (Ib), or a pharmaceutically acceptable salt or solvate thereof, wherein Z¹ is N; and Z² and Z³ are CR⁵. In some embodiments is a compound of Formula (Ia) or (Ib), or a pharmaceutically acceptable salt or solvate thereof, wherein Z² is N; and Z¹ and Z³ are CR⁵. In some embodiments is a compound of Formula (Ia) or (Ib), or a pharmaceutically acceptable salt or solvate thereof, wherein Z³ is N; and Z¹ and Z² are CR⁵. In some embodiments is a compound of Formula (Ia) or (Ib), or a pharmaceutically acceptable salt or solvate thereof, wherein Z¹ is CR⁵; and Z² and Z³ are N. In some embodiments is a compound of Formula (Ia) or (Ib), or a pharmaceutically acceptable salt or solvate thereof, wherein Z² is CR⁵; and Z¹ and Z³ are N. In some embodiments is a compound of Formula (Ia) or (Ib), or a pharmaceutically acceptable salt or solvate thereof, wherein Z³ is CR⁵; and Z¹ and Z² are N. In some embodiments is a compound of Formula (Ia) or (Ib), or a pharmaceutically acceptable salt or solvate thereof, wherein each R⁵ is independently selected from hydrogen, halogen, C₁₋₆alkyl, and —OR¹⁰. In some embodiments is a compound of Formula (Ia) or (Ib), or a pharmaceutically acceptable salt or solvate thereof, wherein each R⁵ is H. In some embodiments is a compound of Formula (Ia) or (Ib), or a pharmaceutically acceptable salt or solvate thereof, wherein Z¹, Z², and Z³ are C(H). In some embodiments is a compound of Formula (Ia) or (Ib), or a pharmaceutically acceptable salt or solvate thereof, wherein Z¹ is N; and Z² and Z³ are C(H). In some embodiments is a compound of Formula (Ia) or (Ib), or a pharmaceutically acceptable salt or solvate thereof, wherein Z² is N; and Z¹ and Z³ are C(H). In some embodiments is a compound of Formula (Ia) or (Ib), or a pharmaceutically acceptable salt or solvate thereof, wherein Z³ is N; and Z¹ and Z² are C(H). In some embodiments is a compound of Formula (Ia) or (Ib), or a pharmaceutically acceptable salt or solvate thereof, wherein Z¹ is C(H); and Z² and Z³ are N. In some embodiments is a compound of Formula (Ia) or (Ib), or a pharmaceutically acceptable salt or solvate thereof, wherein Z² is C(H); and Z¹ and Z³ are N. In some embodiments is a compound of Formula (Ia) or (Ib), or a pharmaceutically acceptable salt or solvate thereof, wherein Z³ is C(H); and Z¹ and Z² are N.

In some embodiments is a compound of Formula (Ia) or (Ib), or a pharmaceutically acceptable salt or solvate thereof, wherein Z⁴ is C(H).

In some embodiments is a compound of Formula (Ia) or (Ib), or a pharmaceutically acceptable salt or solvate thereof, wherein each R³ is independently selected from hydrogen, halogen, —CN, C₁₋₆alkyl, C₁₋₆haloalkyl, and —OH. In some embodiments is a compound of Formula (Ia) or (Ib), or a pharmaceutically acceptable salt or solvate thereof, wherein each R³ is independently selected from hydrogen, halogen, —CN, and CF₃. In some embodiments is a compound of Formula (Ia) or (Ib), or a pharmaceutically acceptable salt or solvate thereof, wherein each R³ is independently selected from hydrogen, halogen, and CF₃. In some embodiments is a compound of Formula (Ia) or (Ib), or a pharmaceutically acceptable salt or solvate thereof, wherein each R³ is independently selected from hydrogen and halogen.

In some embodiments is a compound of Formula (Ia) or (Ib), or a pharmaceutically acceptable salt or solvate thereof, wherein L¹ is a bond. In some embodiments is a compound of Formula (Ia) or (Ib), or a pharmaceutically acceptable salt or solvate thereof, wherein L¹ is —O—. In some embodiments is a compound of Formula (Ia) or (Ib), or a pharmaceutically acceptable salt or solvate thereof, wherein L¹ is —N(R¹⁰)—. In some embodiments is a compound of Formula (Ia) or (Ib), or a pharmaceutically acceptable salt or solvate thereof, wherein L¹ is —N(H)—. In some embodiments is a compound of Formula (Ia) or (Ib), or a pharmaceutically acceptable salt or solvate thereof, wherein L¹ is —C(R¹⁰)(R¹¹)N(R¹⁰)—. In some embodiments is a compound of Formula (Ia) or (Ib), or a pharmaceutically acceptable salt or solvate thereof, wherein L¹ is —CH₂N(H)—. In some embodiments is a compound of Formula (Ia) or (Ib), or a pharmaceutically acceptable salt or solvate thereof, wherein^{L} is —N(R¹⁰)C(R¹⁰)(R¹¹)—. In some embodiments is a compound of Formula (Ia) or (Ib), or a pharmaceutically acceptable salt or solvate thereof, wherein L¹ is —N(H)CH₂—.

In some embodiments is a compound of Formula (Ia) or (Ib), or a pharmaceutically acceptable salt or solvate thereof, wherein R¹ is C₂₋₉heterocycloalkyl optionally substituted with one, two, three, four, or five R². In some embodiments is a compound of Formula (Ia) or (Ib), or a pharmaceutically acceptable salt or solvate thereof, wherein R¹ is C₂₋₉heterocycloalkyl optionally substituted with one, two, or three R². In some embodiments is a compound of Formula (Ia) or (Ib), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, and aziridinyl, wherein piperidinyl, piperazinyl, morpholinyl, tetrahydropyranyl, tetrahydrofuranyl, pyrrolidinyl, oxetanyl, azetidinyl, aziridinyl, azepanyl, and diazepanyl are optionally substituted with one, two, or three R². In some embodiments is a compound of Formula (Ia) or (Ib), or a pharmaceutically acceptable salt or solvate thereof, wherein R¹ is C₂₋₉heterocycloalkyl selected from thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane,2,6-diazaspiro[3.3]heptane, and 2-oxa-6-azaspiro[3.3]heptane, wherein thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane, 2,6-diazaspiro[3.3]heptane, and 2-oxa-6-azaspiro[3.3]heptane are optionally substituted with one, two, or three R². In some embodiments is a compound of Formula (Ia) or (Ib), or a pharmaceutically acceptable salt or solvate thereof, wherein R¹ is C₂₋₉heterocycloalkyl selected from thiomorpholine, 2-oxa-7-azaspiro[3.5]nonane, 7-oxa-2-azaspiro[3.5]nonane, 2,7-diazaspiro[3.5]nonane, 2,6-diazaspiro[3.3]heptane, wherein thiomorpholine, 2-oxa-7-azaspiro[3.5]nonane, 7-oxa-2-azaspiro[3.5]nonane, 2,7-diazaspiro[3.5]nonane, and 2,6-diazaspiro[3.3]heptane are optionally substituted with one, two, or three R². In some embodiments is a compound of Formula (Ia) or (Ib), or a pharmaceutically acceptable salt or solvate thereof, wherein R¹ is

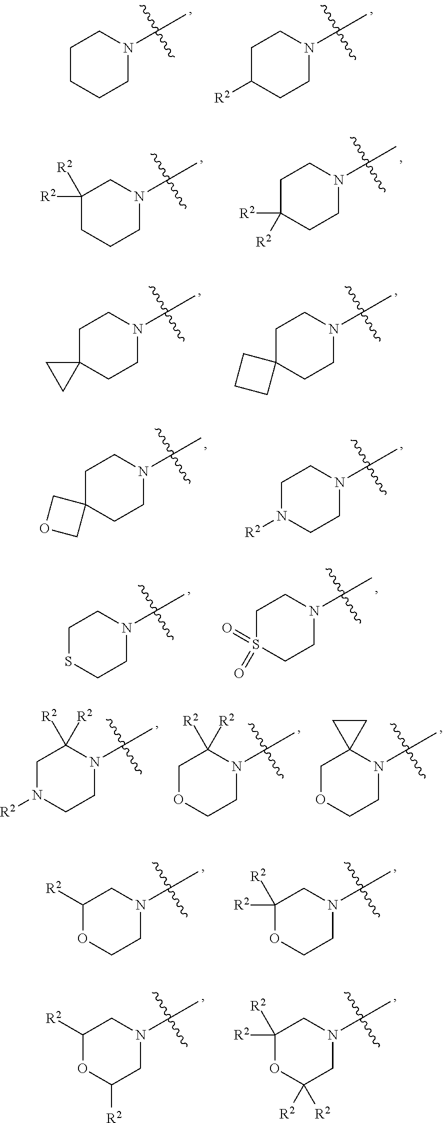

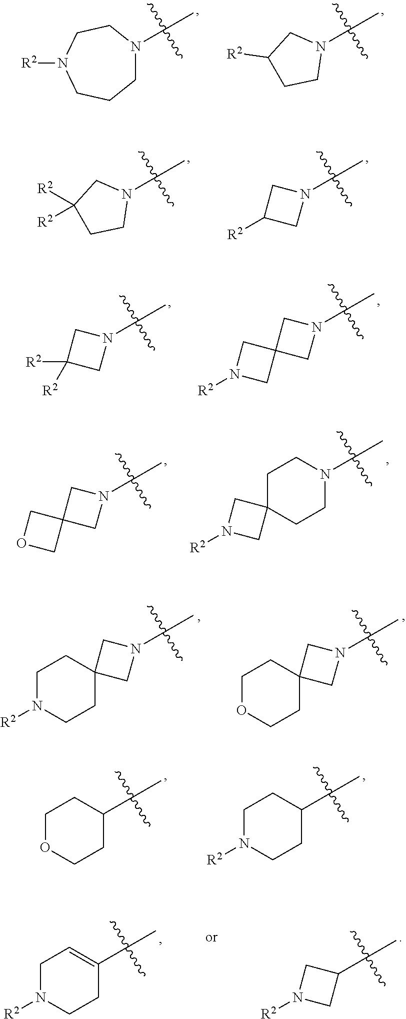

In some embodiments is a compound of Formula (Ia) or (Ib), or a pharmaceutically acceptable salt or solvate thereof, wherein each R² is independently selected from C₁₋₆alkyl, C₁₋₆ haloalkyl, —CN, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (Ia) or (Ib), or a pharmaceutically acceptable salt or solvate thereof, wherein R¹ is

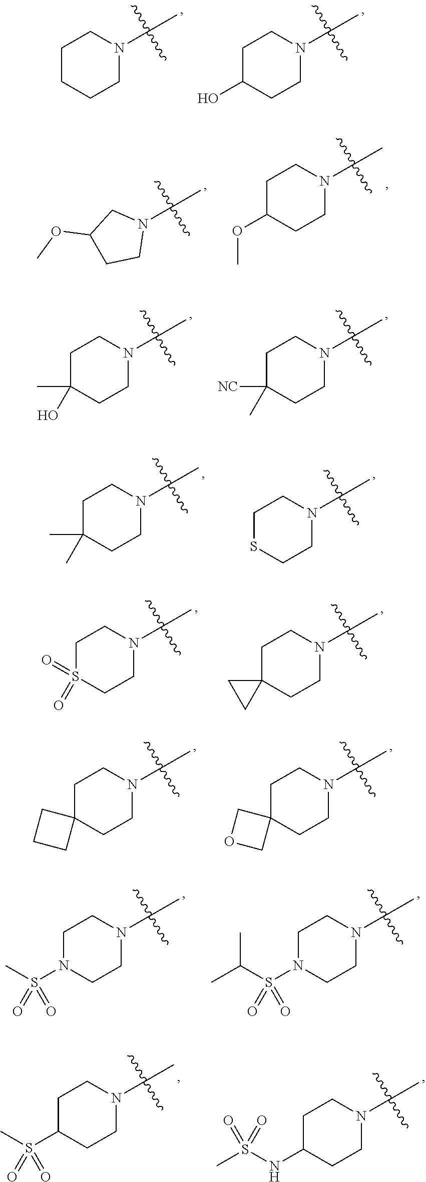

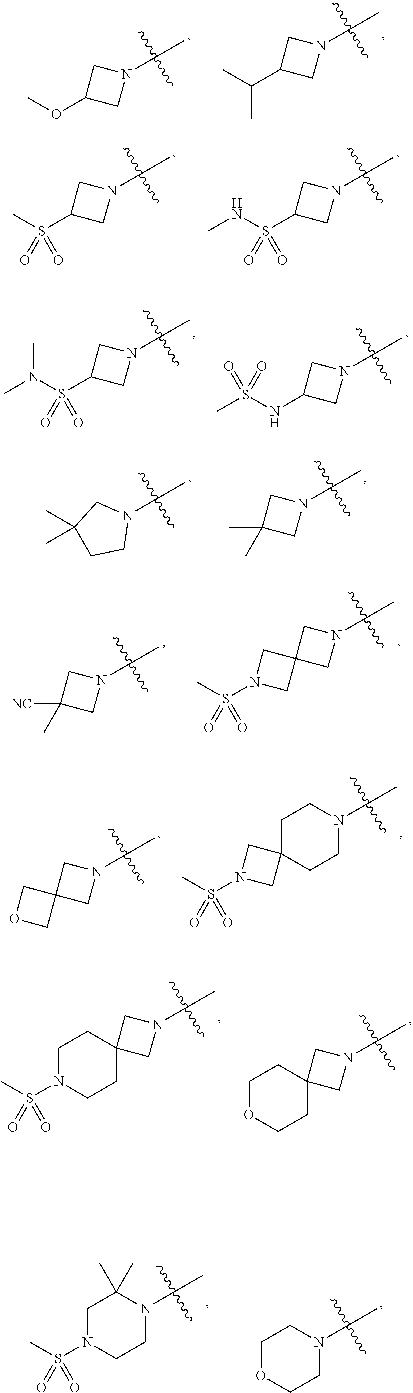

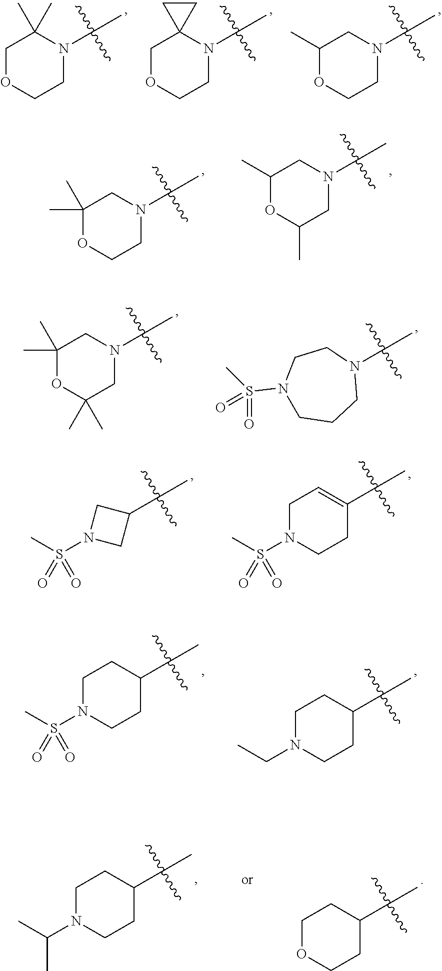

In some embodiments is a compound of Formula (Ia) or (Ib), or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R². In some embodiments is a compound of Formula (Ia) or (Ib), or a pharmaceutically acceptable salt or solvate thereof, wherein R¹ is phenyl substituted with one, two, or three R². In some embodiments is a compound of Formula (Ia) or (Ib), or a pharmaceutically acceptable salt or solvate thereof, wherein R¹ is C₁₋₉heteroaryl substituted with one, two, or three R². In some embodiments is a compound of Formula (Ia) or (Ib), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R².

In some embodiments is a compound of Formula (Ia) or (Ib), or a pharmaceutically acceptable salt or solvate thereof, wherein each R² is independently selected from halogen, —CN, C₁₋₆alkyl, C₁₋₆ haloalkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹). In some embodiments is a compound of Formula (Ia) or (Ib), or a pharmaceutically acceptable salt or solvate thereof, wherein each R² is independently selected from halogen, —CN, C₁₋₆alkyl, —OH, —N(H)S(O)₂CH₃, —S(O)₂CH₃, and —S(O)₂NH₂.

In some embodiments is a compound of Formula (II), or a pharmaceutically acceptable salt or solvate thereof:

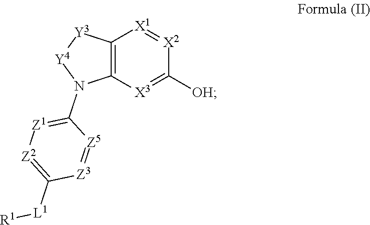

wherein:
X¹, X², and X³ are each independently CR³ or N;
Y³ is C(O), C(R⁴)₂, N(R⁶), O, or S;
Y⁴ is C(O), C(R⁴)₂, or N(R⁶), wherein at least one of Y³ and Y⁴ is C(O);
Z¹ is CR⁵ or N;
Z² is CR⁵ or N;
Z³ is CR⁵ or N;
Z⁵ is CR⁵ or N;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁴ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
R⁶ is selected from hydrogen, C₁₋₆alkyl, C₁₋₆ haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —C(O)R¹³, —S(O)R¹³, —S(O)₂R¹³, —C(O)OR¹⁰, —S(O)₂OR¹⁰, —C(O)N(R¹⁰)(R¹¹), —S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, C₁₋₆alkyl, C₁₋₆haloalkyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —C(O)N(R¹⁰)(R¹¹), —S(O)R¹³, —S(O)₂R¹³, —S(O)₂N(R¹⁰)(R¹¹), —OC(O)N(R¹⁰)(R¹¹), —N(R¹²)C(O)R¹³, —N(R¹²)C(O)OR¹³, and —N(R¹²)C(O)N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl.

In some embodiments is a compound of Formula (II), or a pharmaceutically acceptable salt or solvate thereof, wherein X¹, X², and X³ are each CR³. In some embodiments is a compound of Formula (II), or a pharmaceutically acceptable salt or solvate thereof, wherein X¹, X², and X³ are each CR³ and each R³ is independently selected from hydrogen, halogen, C₁₋₆alkyl, C₁₋₆haloalkyl, and —OR¹⁰. In some embodiments is a compound of Formula (II), or a pharmaceutically acceptable salt or solvate thereof, wherein X¹, X², and X³ are each CR³ and each R³ is independently selected from hydrogen, halogen, C₁₋₆alkyl, and C₁₋₆haloalkyl. In some embodiments is a compound of Formula (II), or a pharmaceutically acceptable salt or solvate thereof, wherein X¹, X², and X³ are each CR³ and each R³ is independently selected from hydrogen, halogen, and C₁₋₆haloalkyl. In some embodiments is a compound of Formula (II), or a pharmaceutically acceptable salt or solvate thereof, wherein X¹, X², and X³ are each CR³ and each R³ is independently selected from hydrogen, halogen, —CN, C₁₋₆alkyl, C₁₋₆haloalkyl, and —OH. In some embodiments is a compound of Formula (II), or a pharmaceutically acceptable salt or solvate thereof, wherein X¹, X², and X³ are each CR³ and each R³ is independently selected from hydrogen, halogen, —CN, and CF₃. In some embodiments is a compound of Formula (II), or a pharmaceutically acceptable salt or solvate thereof, wherein X¹ is C(H), X² is C(H), and X³ is C(CF₃). In some embodiments is a compound of Formula (II), or a pharmaceutically acceptable salt or solvate thereof, wherein X¹ is C(F), X² is C(H), and X³ is C(CF₃). In some embodiments is a compound of Formula (II), or a pharmaceutically acceptable salt or solvate thereof, wherein X¹ is C(CF₃), X² is C(F), and X³ is C(H). In some embodiments is a compound of Formula (II), or a pharmaceutically acceptable salt or solvate thereof, wherein X¹ is C(H), X² is C(F), and X³ is C(CF₃). In some embodiments is a compound of Formula (II), or a pharmaceutically acceptable salt or solvate thereof, wherein X¹ is C(Cl), X² is C(H), and X³ is C(CF₃). In some embodiments is a compound of Formula (II), or a pharmaceutically acceptable salt or solvate thereof, wherein X¹ is C(H), X² is C(F), and X³ is C(CN). In some embodiments is a compound of Formula (II), or a pharmaceutically acceptable salt or solvate thereof, wherein X¹ is C(H), X² is C(H), and X³ is C(F). In some embodiments is a compound of Formula (II), or a pharmaceutically acceptable salt or solvate thereof, wherein X¹ is C(H), X² is C(F), and X³ is C(H). In some embodiments is a compound of Formula (II), or a pharmaceutically acceptable salt or solvate thereof, wherein X¹ is C(F), X² is C(H), and X³ is C(H). In some embodiments is a compound of Formula (II), or a pharmaceutically acceptable salt or solvate thereof, wherein X¹ is C(H), X² is C(F), and X³ is C(F). In some embodiments is a compound of Formula (II), or a pharmaceutically acceptable salt or solvate thereof, wherein X¹ is C(F), X² is C(H), and X³ is C(F). In some embodiments is a compound of Formula (II), or a pharmaceutically acceptable salt or solvate thereof, wherein X¹ is C(F), X² is C(F), and X³ is C(H). In some embodiments is a compound of Formula (II), or a pharmaceutically acceptable salt or solvate thereof, wherein X¹ is C(H), X² is C(H), and X³ is C(Cl). In some embodiments is a compound of Formula (II), or a pharmaceutically acceptable salt or solvate thereof, wherein X¹ is C(Cl), X² is C(F), and X³ is C(H). In some embodiments is a compound of Formula (II), or a pharmaceutically acceptable salt or solvate thereof, wherein X¹ is C(H), X² is C(F), and X³ is C(CH₃).

In some embodiments is a compound of Formula (II), or a pharmaceutically acceptable salt or solvate thereof, wherein Y⁴ is C(O) and Y³ is N(R⁶). In some embodiments is a compound of Formula (II), or a pharmaceutically acceptable salt or solvate thereof, wherein Y⁴ is C(O), Y³ is N(R⁶) and R⁶ is selected from hydrogen and C₁₋₆alkyl optionally substituted with one, two, or three groups selected from halogen, —CN, C₁₋₆alkyl, C₁₋₆ haloalkyl, —OR¹⁰, and —N(R¹⁰)(R¹¹). In some embodiments is a compound of Formula (II), or a pharmaceutically acceptable salt or solvate thereof, wherein Y⁴ is C(O), Y³ is N(R⁶) and R⁶ is hydrogen. In some embodiments is a compound of Formula (II), or a pharmaceutically acceptable salt or solvate thereof, wherein Y⁴ is C(O), Y³ is N(R⁶) and R⁶ is C₁₋₆alkyl optionally substituted with one, two, or three groups selected from halogen, —CN, C₁₋₆alkyl, C₁₋₆haloalkyl, —OR¹⁰, and —N(R¹⁰)(R¹¹). In some embodiments is a compound of Formula (II), or a pharmaceutically acceptable salt or solvate thereof, wherein Y⁴ is C(O), Y³ is N(R⁶) and R⁶ is C₁₋₆alkyl optionally substituted with —OH. In some embodiments is a compound of Formula (II), or a pharmaceutically acceptable salt or solvate thereof, wherein Y⁴ is C(O) and Y³ is C(R⁴)₂. In some embodiments is a compound of Formula (II), or a pharmaceutically acceptable salt or solvate thereof, wherein Y⁴ is C(O), Y³ is C(R⁴)₂ and each R⁴ is hydrogen or C₁₋₆alkyl. In some embodiments is a compound of Formula (II), or a pharmaceutically acceptable salt or solvate thereof, wherein Y⁴ is C(O), Y³ is C(R⁴)₂ and each R⁴ is hydrogen.

In some embodiments is a compound of Formula (II), or a pharmaceutically acceptable salt or solvate thereof, wherein Y³ is C(O) and Y⁴ is N(R⁶). In some embodiments is a compound of Formula (II), or a pharmaceutically acceptable salt or solvate thereof, wherein Y³ is C(O), Y⁴ is N(R⁶) and R⁶ is selected from hydrogen and C₁₋₆alkyl optionally substituted with one, two, or three groups selected from halogen, —CN, C₁₋₆alkyl, C₁₋₆ haloalkyl, —OR¹⁰, and —N(R¹⁰)(R¹¹). In some embodiments is a compound of Formula (II), or a pharmaceutically acceptable salt or solvate thereof, wherein Y³ is C(O), Y⁴ is N(R⁶) and R⁶ is hydrogen. In some embodiments is a compound of Formula (II), or a pharmaceutically acceptable salt or solvate thereof, wherein Y³ is C(O), Y⁴ is N(R⁶) and R⁶ is C₁₋₆alkyl optionally substituted with one, two, or three groups selected from halogen, —CN, C₁₋₆alkyl, C₁₋₆haloalkyl, —OR¹⁰, and —N(R¹⁰)(R¹¹). In some embodiments is a compound of Formula (II), or a pharmaceutically acceptable salt or solvate thereof, wherein Y³ is C(O), Y⁴ is N(R⁶) and R⁶ is C₁₋₆alkyl optionally substituted with —OH. In some embodiments is a compound of Formula (II), or a pharmaceutically acceptable salt or solvate thereof, wherein Y³ is C(O) and Y⁴ is C(R⁴)₂. In some embodiments is a compound of Formula (II), or a pharmaceutically acceptable salt or solvate thereof, wherein Y³ is C(O), Y⁴ is C(R⁴)₂ and each R⁴ is hydrogen or C₁₋₆alkyl. In some embodiments is a compound of Formula (II), or a pharmaceutically acceptable salt or solvate thereof, wherein Y³ is C(O), Y⁴ is C(R⁴)₂ and each R⁴ is hydrogen.

In some embodiments is a compound of Formula (II), or a pharmaceutically acceptable salt or solvate thereof, wherein Y³ and Y⁴ are C(O),

In some embodiments is a compound of Formula (IIa), or a pharmaceutically acceptable salt or solvate thereof:

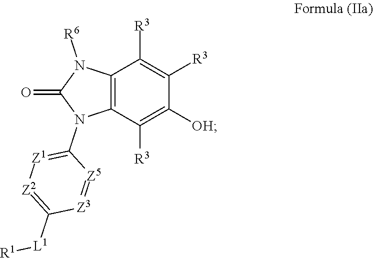

wherein:
Z¹ is CR⁵ or N;
Z² is CR⁵ or N;
Z³ is CR⁵ or N;
Z⁵ is CR⁵ or N;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
R⁶ is selected from hydrogen, C₁₋₆alkyl, C₁₋₆ haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —C(O)R¹³, —S(O)R¹³, —S(O)₂R¹³, —C(O)OR¹⁰, —S(O)₂OR¹⁰, —C(O)N(R¹⁰)(R¹¹), —S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, C₁₋₆alkyl, C₁₋₆haloalkyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —C(O)N(R¹⁰)(R¹¹), —S(O)R¹³, —S(O)₂R¹³, —S(O)₂N(R¹⁰)(R¹¹), —OC(O)N(R¹⁰)(R¹¹), —N(R¹²)C(O)R¹³, —N(R¹²)C(O)OR¹³, and —N(R¹²)C(O)N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (IIa), or a pharmaceutically acceptable salt or solvate thereof, wherein R⁶ is selected from hydrogen and C₁₋₆alkyl optionally substituted with one, two, or three groups selected from halogen, —CN, C₁₋₆alkyl, C₁₋₆haloalkyl, —OR¹⁰, and —N(R¹⁰)(R¹¹). In some embodiments is a compound of Formula (IIa), or a pharmaceutically acceptable salt or solvate thereof, wherein R⁶ is hydrogen. In some embodiments is a compound of Formula (IIa), or a pharmaceutically acceptable salt or solvate thereof, wherein R⁶ is C₁₋₆alkyl optionally substituted with one, two, or three groups selected from halogen, —CN, C₁₋₆alkyl, C₁₋₆haloalkyl, —OR¹⁰, and —N(R¹⁰)(R¹¹). In some embodiments is a compound of Formula (IIa), or a pharmaceutically acceptable salt or solvate thereof, wherein R⁶ is C₁₋₆alkyl optionally substituted with —OH.

In some embodiments is a compound of Formula (IIb), or a pharmaceutically acceptable salt or solvate thereof:

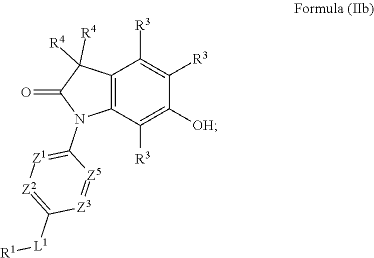

wherein:
Z¹ is CR⁵ or N;
Z² is CR⁵ or N;
Z³ is CR⁵ or N;
Z⁵ is CR⁵ or N;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁴ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (IIb), or a pharmaceutically acceptable salt or solvate thereof, wherein each R⁴ is independently selected from hydrogen and C₁₋₆alkyl. In some embodiments is a compound of Formula (IIb), or a pharmaceutically acceptable salt or solvate thereof, wherein each R⁴ is hydrogen. In some embodiments is a compound of Formula (IIb), or a pharmaceutically acceptable salt or solvate thereof, wherein each R⁴ is C₁₋₆alkyl.

In some embodiments is a compound of Formula (IIa) or (IIb), or a pharmaceutically acceptable salt or solvate thereof, wherein each R³ is independently selected from hydrogen, halogen, —CN, C₁₋₆alkyl, C₁₋₆haloalkyl, and —OH. In some embodiments is a compound of Formula (IIa) or (IIb), or a pharmaceutically acceptable salt or solvate thereof, wherein each R³ is independently selected from hydrogen, halogen, —CN, and CF₃. In some embodiments is a compound of Formula (IIa) or (IIb), or a pharmaceutically acceptable salt or solvate thereof, wherein each R³ is independently selected from hydrogen, halogen, and CF₃. In some embodiments is a compound of Formula (IIa) or (IIb), or a pharmaceutically acceptable salt or solvate thereof, wherein each R³ is independently selected from hydrogen and halogen.

In some embodiments is a compound of Formula (II), (IIa), or (IIb), or a pharmaceutically acceptable salt or solvate thereof, wherein Z⁵ is CR⁵. In some embodiments is a compound of Formula (II), (IIa), or (IIb), or a pharmaceutically acceptable salt or solvate thereof, wherein Z⁵ is C(H). In some embodiments is a compound of Formula (II), (IIa), or (IIb), or a pharmaceutically acceptable salt or solvate thereof, wherein Z⁵ is N.

In some embodiments is a compound of Formula (II), (IIa), or (IIb), or a pharmaceutically acceptable salt or solvate thereof, wherein Z¹, Z², and Z³ are CR⁵. In some embodiments is a compound of Formula (II), (IIa), or (IIb), or a pharmaceutically acceptable salt or solvate thereof, wherein Z¹ is N; and Z² and Z³ are CR⁵. In some embodiments is a compound of Formula (II), (IIa), or (IIb), or a pharmaceutically acceptable salt or solvate thereof, wherein Z² is N; and Z¹ and Z³ are CR⁵. In some embodiments is a compound of Formula (II), (IIa), or (IIb), or a pharmaceutically acceptable salt or solvate thereof, wherein Z³ is N; and Z¹ and Z² are CR⁵. In some embodiments is a compound of Formula (II), (IIa), or (IIb), or a pharmaceutically acceptable salt or solvate thereof, wherein Z¹ is CR⁵; and Z² and Z³ are N. In some embodiments is a compound of Formula (II), (IIa), or (IIb), or a pharmaceutically acceptable salt or solvate thereof, wherein Z² is CR⁵; and Z¹ and Z³ are N. In some embodiments is a compound of Formula (II), (IIa), or (IIb), or a pharmaceutically acceptable salt or solvate thereof, wherein Z³ is CR⁵; and Z¹ and Z² are N. In some embodiments is a compound of Formula (II), (IIa), or (IIb), or a pharmaceutically acceptable salt or solvate thereof, wherein each R⁵ is independently selected from hydrogen, halogen, C₁₋₆ alkyl, and —OR¹⁰. In some embodiments is a compound of Formula (II), (IIa), or (IIb), or a pharmaceutically acceptable salt or solvate thereof, wherein each R⁵ is H. In some embodiments is a compound of Formula (II), (IIa), or (IIb), or a pharmaceutically acceptable salt or solvate thereof, wherein Z¹, Z², and Z³ are C(H). In some embodiments is a compound of Formula (II), (IIa), or (IIb), or a pharmaceutically acceptable salt or solvate thereof, wherein Z¹ is N; and Z² and Z³ are C(H). In some embodiments is a compound of Formula (II), (IIa), or (IIb), or a pharmaceutically acceptable salt or solvate thereof, wherein Z² is N; and Z¹ and Z³ are C(H). In some embodiments is a compound of Formula (II), (IIa), or (IIb), or a pharmaceutically acceptable salt or solvate thereof, wherein Z³ is N; and Z¹ and Z² are C(H). In some embodiments is a compound of Formula (II), (IIa), or (IIb), or a pharmaceutically acceptable salt or solvate thereof, wherein Z¹ is C(H); and Z² and Z³ are N. In some embodiments is a compound of Formula (II), (IIa), or (IIb), or a pharmaceutically acceptable salt or solvate thereof, wherein Z² is C(H); and Z¹ and Z³ are N. In some embodiments is a compound of Formula (II), (IIa), or (IIb), or a pharmaceutically acceptable salt or solvate thereof, wherein Z³ is C(H); and Z¹ and Z² are N.

In some embodiments is a compound of Formula (II), (IIa), or (IIb), or a pharmaceutically acceptable salt or solvate thereof, wherein L¹ is a bond. In some embodiments is a compound of Formula (II), (IIa), or (IIb), or a pharmaceutically acceptable salt or solvate thereof, wherein L¹ is —O—. In some embodiments is a compound of Formula (II), (IIa), or (IIb), or a pharmaceutically acceptable salt or solvate thereof, wherein L¹ is —N(R¹⁰)—. In some embodiments is a compound of Formula (II), (IIa), or (IIb), or a pharmaceutically acceptable salt or solvate thereof, wherein L¹ is —N(H)—. In some embodiments is a compound of Formula (II), (IIa), or (IIb), or a pharmaceutically acceptable salt or solvate thereof, wherein L¹ is —C(R¹⁰)(R¹¹)N(R¹⁰)—. In some embodiments is a compound of Formula (II), (IIa), or (IIb), or a pharmaceutically acceptable salt or solvate thereof, wherein L¹ is —CH₂N(H)—. In some embodiments is a compound of Formula (II), (IIa), or (IIb), or a pharmaceutically acceptable salt or solvate thereof, wherein L¹ is —N(R¹⁰)C(R¹⁰)(R¹¹)—. In some embodiments is a compound of Formula (II), (IIa), or (IIb), or a pharmaceutically acceptable salt or solvate thereof, wherein L¹ is —N(H)CH₂—.

In some embodiments is a compound of Formula (II), (IIa), or (IIb), or a pharmaceutically acceptable salt or solvate thereof, wherein R¹ is C₂₋₉heterocycloalkyl optionally substituted with one, two, three, four, or five R². In some embodiments is a compound of Formula (II), (IIa), or (IIb), or a pharmaceutically acceptable salt or solvate thereof, wherein R¹ is C₂₋₉heterocycloalkyl optionally substituted with one, two, or three R². In some embodiments is a compound of Formula (II), (IIa), or (IIb), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, and aziridinyl, wherein piperidinyl, piperazinyl, morpholinyl, tetrahydropyranyl, tetrahydrofuranyl, pyrrolidinyl, oxetanyl, azetidinyl, aziridinyl, azepanyl, and diazepanyl are optionally substituted with one, two, or three R². In some embodiments is a compound of Formula (II), (IIa), or (IIb), or a pharmaceutically acceptable salt or solvate thereof, wherein R¹ is C₂₋₉heterocycloalkyl selected from thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane,2,6-diazaspiro[3.3]heptane, and 2-oxa-6-azaspiro[3.3]heptane, wherein thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane, 2,6-diazaspiro[3.3]heptane, and 2-oxa-6-azaspiro[3.3]heptane are optionally substituted with one, two, or three R². In some embodiments is a compound of Formula (II), (IIa), or (IIb), or a pharmaceutically acceptable salt or solvate thereof, wherein R¹ is C₂₋₉heterocycloalkyl selected from thiomorpholine, 2-oxa-7-azaspiro[3.5]nonane, 7-oxa-2-azaspiro[3.5]nonane, 2,7-diazaspiro[3.5]nonane, 2,6-diazaspiro[3.3]heptane, wherein thiomorpholine, 2-oxa-7-azaspiro[3.5]nonane, 7-oxa-2-azaspiro[3.5]nonane, 2,7-diazaspiro[3.5]nonane, and 2,6-diazaspiro[3.3]heptane are optionally substituted with one, two, or three R². In some embodiments is a compound of Formula (II), (IIa), or (IIb), or a pharmaceutically acceptable salt or solvate thereof, wherein R¹ is

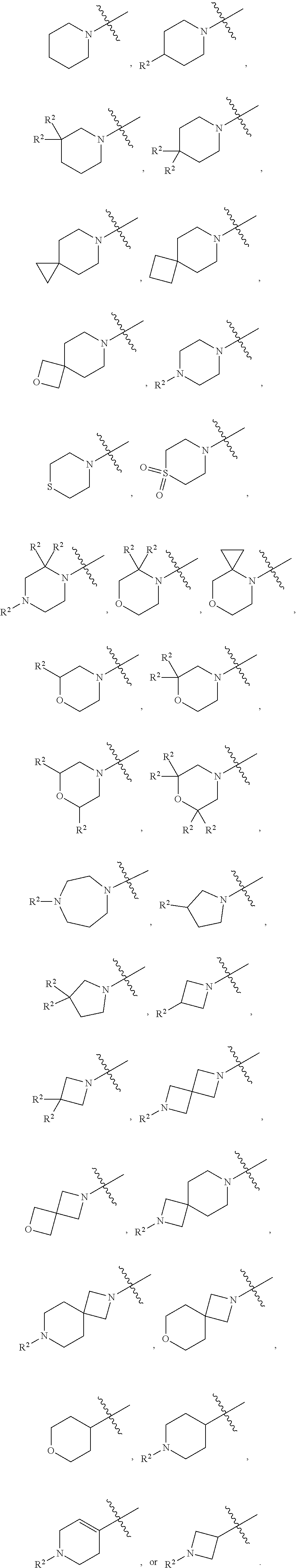

In some embodiments is a compound of Formula (II), (IIa), or (IIb), or a pharmaceutically acceptable salt or solvate thereof, wherein each R² is independently selected from C₁₋₆alkyl, C₁₋₆ haloalkyl, —CN, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (II), (IIa), or (IIb), or a pharmaceutically acceptable salt or solvate thereof, wherein R¹ is

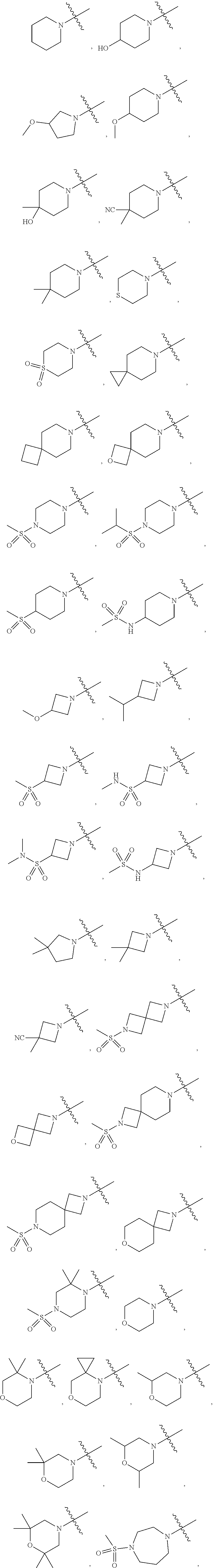

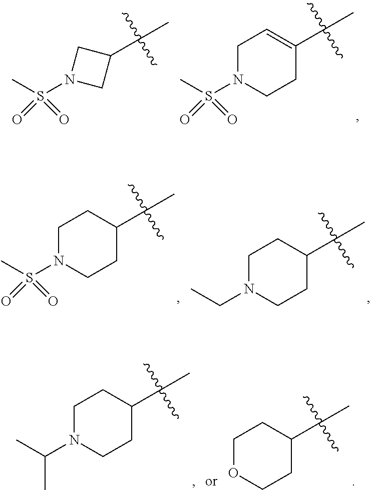

In some embodiments is a compound of Formula (II), (Ia), or (IIb), or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R². In some embodiments is a compound of Formula (II), (IIa), or (IIb), or a pharmaceutically acceptable salt or solvate thereof, wherein R¹ is phenyl substituted with one, two, or three R². In some embodiments is a compound of Formula (II), (IIa), or (IIb), or a pharmaceutically acceptable salt or solvate thereof, wherein R¹ is C₁₋₉heteroaryl substituted with one, two, or three R². In some embodiments is a compound of Formula (II), (IIa), or (IIb), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R².

In some embodiments is a compound of Formula (II), (IIa), or (IIb), or a pharmaceutically acceptable salt or solvate thereof, wherein each R² is independently selected from halogen, —CN, C₁₋₆alkyl, C₁₋₆ haloalkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹). In some embodiments is a compound of Formula (II), (IIa), or (IIb), or a pharmaceutically acceptable salt or solvate thereof, wherein each R² is independently selected from halogen, —CN, C₁₋₆alkyl, —OH, —N(H)S(O)₂CH₃, —S(O)₂CH₃, and —S(O)₂NH₂.

Any combination of the groups described above for the various variables is contemplated herein. Throughout the specification, groups and substituents thereof are chosen by one skilled in the field to provide stable moieties and compounds.

In some embodiments, compounds described herein include, but are not limited to, those described in Table 1.

TABLE 1

| Compound | Structure | Chemical Name |
|---|---|---|
| 1 | 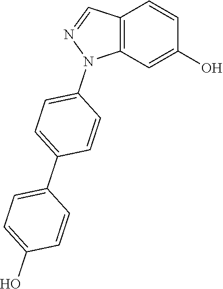 [SMILES: Oc1ccc(-c2ccc(-n3ncc4ccc(O)cc43)cc2)cc1] | 1-(4′-Hydroxy-[1,1′-biphenyl]-4-yl)-1H- indazol-6-ol |
| 2 | 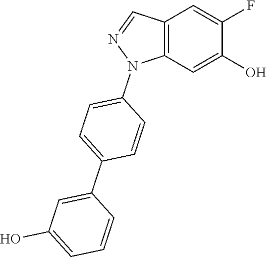 [SMILES: Oc1cccc(-c2ccc(-n3ncc4cc(F)c(O)cc43)cc2)c1] | 5-Fluoro-1-(3′-hydroxy-[1,1′-biphenyl]-4- yl)-1H-indazol-6-ol |
| 2.1 | 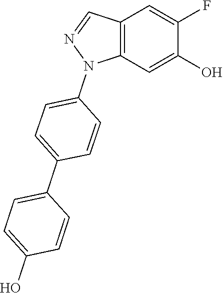 [SMILES: Oc1ccc(-c2ccc(-n3ncc4cc(F)c(O)cc43)cc2)cc1] | 5-Fluoro-1-(4′-hydroxy-[1,1′-biphenyl]-4- yl)-1H-indazol-6-ol |
| 2.2 | 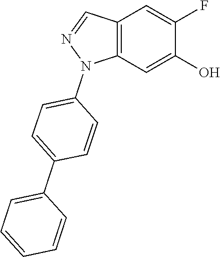 [SMILES: Oc1cc2c(cnn2-c2ccc(-c3ccccc3)cc2)cc1F] | 1-([1,1′-Biphenyl]-4-yl)-5-fluoro-1H- indazol-6-ol |
| 2.3 | 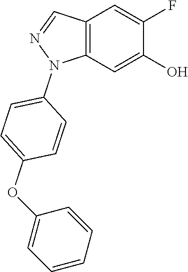 [SMILES: Oc1cc2c(cnn2-c2ccc(Oc3ccccc3)cc2)cc1F] | 5-Fluoro-1-(4-phenoxyphenyl)-1H-indazol- 6-o1 |
| 2.4 | 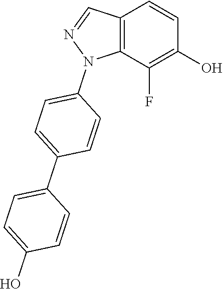 [SMILES: Oc1ccc(-c2ccc(-n3ncc4ccc(O)c(F)c43)cc2)cc1] | 7-Fluoro-1-(4′-hydroxy-[1,1′-biphenyl]-4- yl)-1H-indazol-6-ol |
| 2.5 | 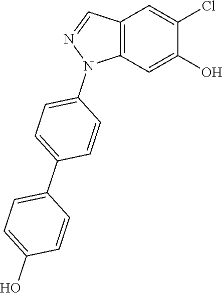 [SMILES: Oc1ccc(-c2ccc(-n3ncc4cc(Cl)c(O)cc43)cc2)cc1] | 5-Chloro-1-(4′-hydroxy-[1,1′-biphenyl]-4- yl)-1H-indazol-6-ol |
| 2.6 | 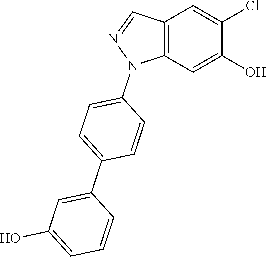 [SMILES: Oc1cccc(-c2ccc(-n3ncc4cc(Cl)c(O)cc43)cc2)c1] | 5-Chloro-1-(3′-hydroxy-[1,1′-biphenyl]-4- yl)-1H-indazol-6-ol |
| 2.7 | 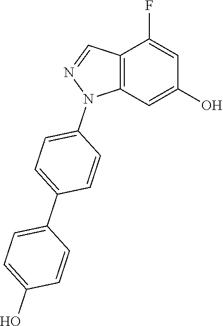 [SMILES: Oc1ccc(-c2ccc(-n3ncc4c(F)cc(O)cc43)cc2)cc1] | 4-Fluoro-1-(4′-hydroxy-[1,1′-biphenyl]-4- yl)-1H-indazol-6-ol |
| 2.8 | 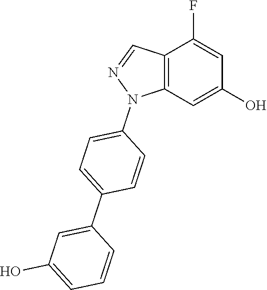 [SMILES: Oc1cccc(-c2ccc(-n3ncc4c(F)cc(O)cc43)cc2)c1] | 4-Fluoro-1-(3′-hydroxy-[1,1′-biphenyl]-4- yl)-1H-indazol-6-ol |
| 2.9 | 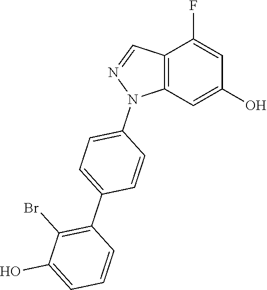 [SMILES: Oc1cc(F)c2cnn(-c3ccc(-c4cccc(O)c4Br)cc3)c2c1] | 1-(2′-Bromo-3′-hydroxy-[1,1′-biphenyl]-4- yl)-4-fluoro-1H-indazol-6-ol |
| 3 | 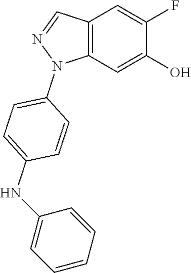 [SMILES: Oc1cc2c(cnn2-c2ccc(Nc3ccccc3)cc2)cc1F] | 5-Fluoro-1-(4-(phenylamino)phenyl)-1H- indazol-6-ol |
| 4 | 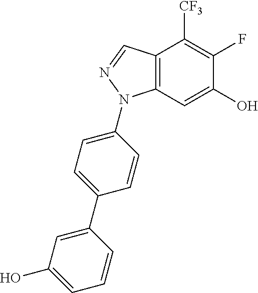 [SMILES: Oc1cccc(-c2ccc(-n3ncc4c(C(F)(F)F)c(F)c(O)cc43)cc2)c1] | 5-Fluoro-1-(3′-hydroxy-[1,1′-biphenyl]-4- yl)-4-(trifluoromethyl)-1H-indazol-6-ol |
| 4.1 | 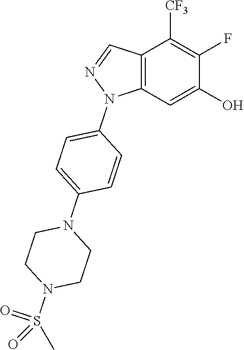 [SMILES: CS(=O)(=O)N1CCN(c2ccc(-n3ncc4c(C(F)(F)F)c(F)c(O)cc43)cc2)CC1] | 5-Fluoro-1-(4-(4-(methylsulfonyl)piperazin- 1-yl)phenyl)-4-(trifluoromethyl)-1H-indazol- 6-ol |
| 4.2 | 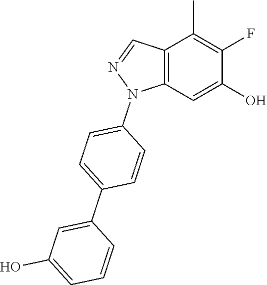 [SMILES: Cc1c(F)c(O)cc2c1cnn2-c1ccc(-c2cccc(O)c2)cc1] | 5-Fluoro-1-(3′-hydroxy-[1,1′-biphenyl]-4- yl)-4-methyl-1H-indazol-6-ol |
| 4.3 | 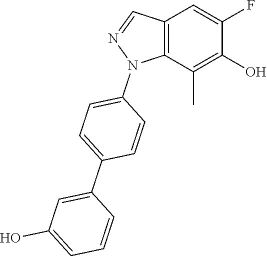 [SMILES: Cc1c(O)c(F)cc2cnn(-c3ccc(-c4cccc(O)c4)cc3)c12] | 5-Fluoro-1-(3′-hydroxy-[1,1′-biphenyl]-4- yl)-7-methyl-1H-indazol-6-ol |
| 5 | 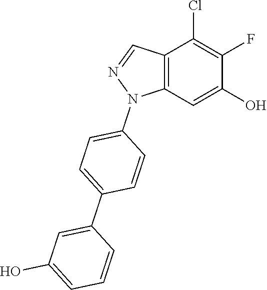 [SMILES: Oc1cccc(-c2ccc(-n3ncc4c(Cl)c(F)c(O)cc43)cc2)c1] | 4-Chloro-5-fluoro-1-(3′-hydroxy-[1,1′- biphenyl]-4-yl)-1H-indazol-6-ol |
| 5.1 | 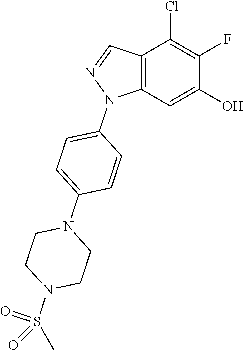 [SMILES: CS(=O)(=O)N1CCN(c2ccc(-n3ncc4c(Cl)c(F)c(O)cc43)cc2)CC1] | 4-Chloro-5-fluoro-1-(4-(4- (methylsulfonyl)piperazin-1-yl)phenyl)-1H- indazol-6-ol |
| 5.2 | 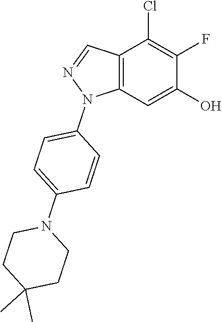 [SMILES: CC1(C)CCN(c2ccc(-n3ncc4c(Cl)c(F)c(O)cc43)cc2)CC1] | 4-Chloro-1-(4-(4,4-dimethylpiperidin-1- yl)phenyl)-5-fluoro-1H-indazol-6-ol |
| 5.3 | 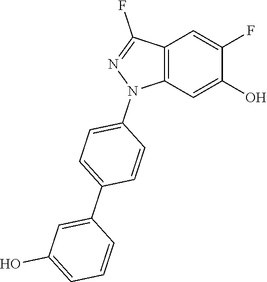 [SMILES: Oc1cccc(-c2ccc(-n3nc(F)c4cc(F)c(O)cc43)cc2)c1] | 3,5-Difluoro-1-(3′-hydroxy-[1,1′-biphenyl]- 4-yl)-1H-indazol-6-ol |
| 5.4 | 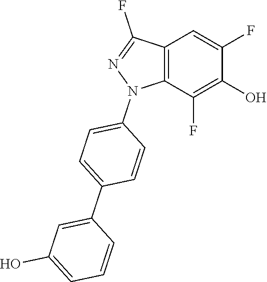 [SMILES: Oc1cccc(-c2ccc(-n3nc(F)c4cc(F)c(O)c(F)c43)cc2)c1] | 3,5,7-Trifluoro-1-(3′-hydroxy-[1,1′- biphenyl]-4-yl)-1H-indazol-6-ol |
| 6 | 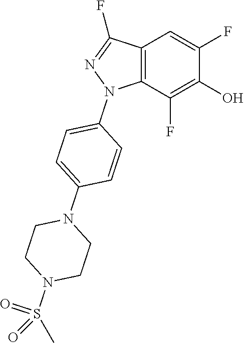 [SMILES: CS(=O)(=O)N1CCN(c2ccc(-n3nc(F)c4cc(F)c(O)c(F)c43)cc2)CC1] | 3,5,7-Trifluoro-1-(4-(4- (methylsulfonyl)piperazin-1-yl)phenyl)-1H- indazol-6-ol |
| 6.1 | 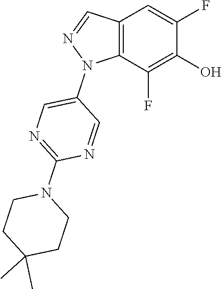 [SMILES: CC1(C)CCN(c2ncc(-n3ncc4cc(F)c(O)c(F)c43)cn2)CC1] | 1-(2-(4,4-Dimethylpiperidin-1-yl)pyrimidin- 5-yl)-5,7-difluoro-1H-indazol-6-ol |
| 6.2 | 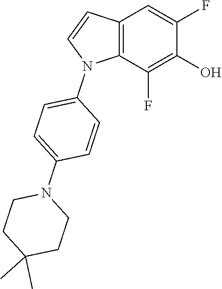 [SMILES: CC1(C)CCN(c2ccc(-n3ccc4cc(F)c(O)c(F)c43)cc2)CC1] | 1-(4-(4,4-Dimethylpiperidin-1-yl)phenyl)- 5,7-difluoro-1H-indol-6-ol |
| 7 | 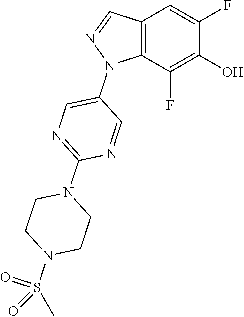 [SMILES: CS(=O)(=O)N1CCN(c2ncc(-n3ncc4cc(F)c(O)c(F)c43)cn2)CC1] | 5,7-Difluoro-1-(2-(4- (methylsulfonyl)piperazin-1-yl)pyrimidin-5- yl)-1H-indazol-6-ol |
| 7.1 | 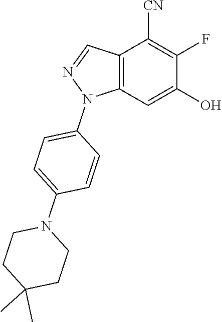 [SMILES: CC1(C)CCN(c2ccc(-n3ncc4c(C#N)c(F)c(O)cc43)cc2)CC1] | 1-(4-(4,4-Dimethylpiperidin-1-yl)phenyl)-5- fluoro-6-hydroxy-1H-indazole-4-carbonitrile |
| 7.2 | 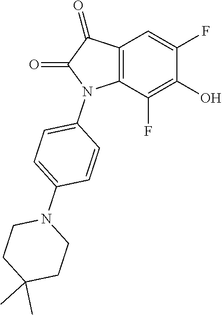 [SMILES: CC1(C)CCN(c2ccc(N3C(=O)C(=O)c4cc(F)c(O)c(F)c43)cc2)CC1] | 1-(4-(4,4-Dimethylpiperidin-1-yl)phenyl)- 5,7-difluoro-6-hydroxyindoline-2,3-dione |
| 7.3 | 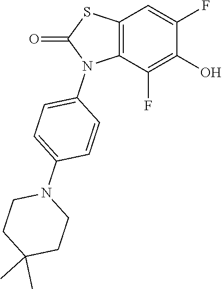 [SMILES: CC1(C)CCN(c2ccc(-n3c(=O)sc4cc(F)c(O)c(F)c43)cc2)CC1] | 3-(4-(4,4-Dimethylpiperidin-1-yl)phenyl)- 4,6-difluoro-5-hydroxybenzo[d]thiazol- 2(3H)-one |
| 7.4 | 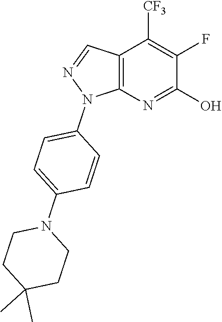 [SMILES: CC1(C)CCN(c2ccc(-n3ncc4c(C(F)(F)F)c(F)c(O)nc43)cc2)CC1] | 1-(4-(4,4-Dimethylpiperidin-1-yl)phenyl)-5- fluoro-4-(trifluoromethyl)-1H-pyrazolo[3,4- b]pyridin-6-ol |
| 8 | 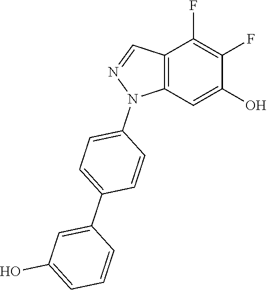 [SMILES: Oc1cccc(-c2ccc(-n3ncc4c(F)c(F)c(O)cc43)cc2)c1] | 4,5-Difluoro-1-(3′-hydroxy-[1,1′-biphenyl]- 4-yl)-1H-indazol-6-ol |
| 9 | 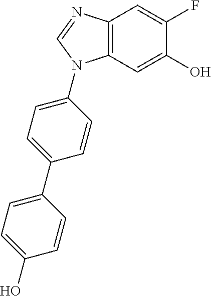 [SMILES: Oc1ccc(-c2ccc(-n3cnc4cc(F)c(O)cc43)cc2)cc1] | 5-Fluoro-1-(4′-hydroxy-[1,1′-biphenyl]-4- yl)-1H-benzo[d]imidazol-6-ol |
| 10 | 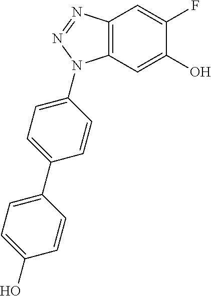 [SMILES: Oc1ccc(-c2ccc(-n3nnc4cc(F)c(O)cc43)cc2)cc1] | 5-Fluoro-1-(4′-hydroxy-[1,1′-biphenyl]-4- yl)-1H-benzo[d][1,2,3]triazol-6-ol |
| 11 | 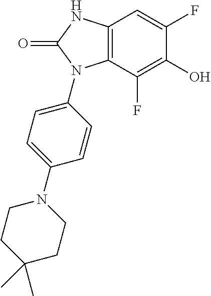 [SMILES: CC1(C)CCN(c2ccc(-n3c(=O)[nH]c4cc(F)c(O)c(F)c43)cc2)CC1] | 1-(4-(4,4-Dimethylpiperidin-1-yl)phenyl)- 5,7-difluoro-6-hydroxy-1H- benzo[d]imidazol-2(3H)-one |
| 12 | 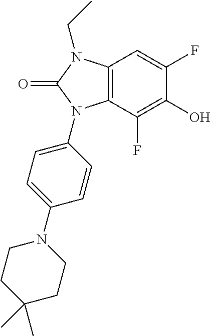 [SMILES: CCn1c(=O)n(-c2ccc(N3CCC(C)(C)CC3)cc2)c2c(F)c(O)c(F)cc21] | 3-(4-(4,4-Dimethylpiperidin-1-yl)phenyl)-1- ethyl-4,6-difluoro-5-hydroxy-1H- benzo[d]imidazol-2(3H)-one |
| 13 | 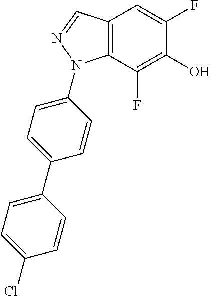 [SMILES: Oc1c(F)cc2cnn(-c3ccc(-c4ccc(Cl)cc4)cc3)c2c1F] | 1-(4′-Chloro-[1,1′-biphenyl]-4-yl)-5,7- difluoro-1H-indazol-6-ol |
| 13.1 | 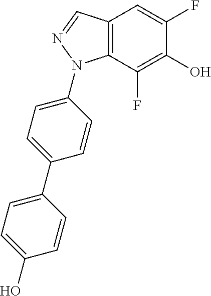 [SMILES: Oc1ccc(-c2ccc(-n3ncc4cc(F)c(O)c(F)c43)cc2)cc1] | 5,7-Difluoro-1-(4′-hydroxy-[1,1′-biphenyl]- 4-yl)-1H-indazol-6-ol |
| 13.2 | 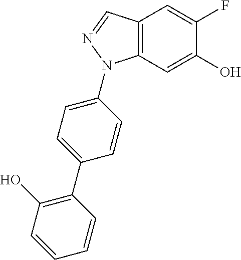 [SMILES: Oc1cc2c(cnn2-c2ccc(-c3ccccc3O)cc2)cc1F] | 5-Fluoro-1-(2′-hydroxy-[1,1′-biphenyl]-4- yl)-1H-indazol-6-ol |
| 13.3 | 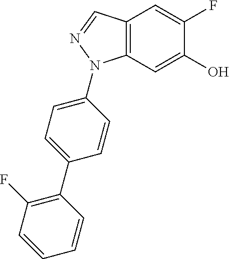 [SMILES: Oc1cc2c(cnn2-c2ccc(-c3ccccc3F)cc2)cc1F] | 5-Fluoro-1-(2′-fluoro-[1,1′-biphenyl]-4-yl)- 1H-indazol-6-ol |
| 13.4 | 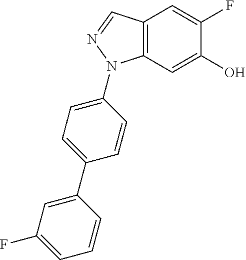 [SMILES: Oc1cc2c(cnn2-c2ccc(-c3cccc(F)c3)cc2)cc1F] | 5-Fluoro-1-(3′-fluoro-[1,1′-biphenyl]-4-yl)- 1H-indazol-6-ol |
| 13.5 | 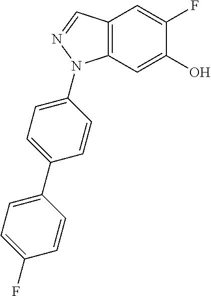 [SMILES: Oc1cc2c(cnn2-c2ccc(-c3ccc(F)cc3)cc2)cc1F] | 5-Fluoro-1-(4′-fluoro-[1,1′-biphenyl]-4-yl)- 1H-indazol-6-ol |
| 13.6 | 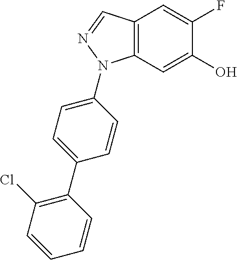 [SMILES: Oc1cc2c(cnn2-c2ccc(-c3ccccc3Cl)cc2)cc1F] | 1-(2′-Chloro-[1,1′-biphenyl]-4-yl)-5-fluoro- 1H-indazol-6-ol |
| 13.7 | 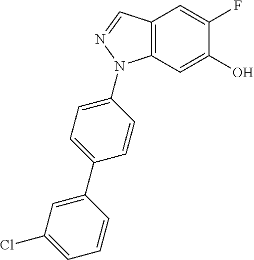 [SMILES: Oc1cc2c(cnn2-c2ccc(-c3cccc(Cl)c3)cc2)cc1F] | 1-(3′-Chloro-[1,1′-biphenyl]-4-yl)-5-fluoro- 1H-indazol-6-ol |
| 13.8 | 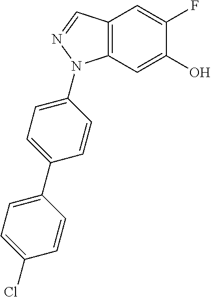 [SMILES: Oc1cc2c(cnn2-c2ccc(-c3ccc(Cl)cc3)cc2)cc1F] | 1-(4′-Chloro-[1,1′-biphenyl]-4-yl)-5-fluoro- 1H-indazol-6-ol |
| 13.9 | 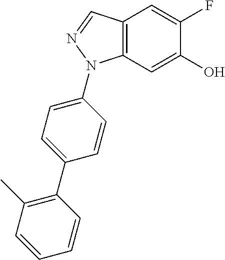 [SMILES: Cc1ccccc1-c1ccc(-n2ncc3cc(F)c(O)cc32)cc1] | 5-Fluoro-1-(2′-methyl-[1,1′-biphenyl]-4-yl)- 1H-indazol-6-ol |
| 13.10 | 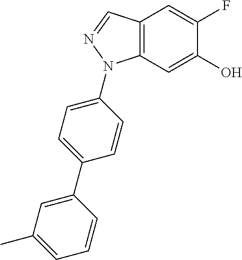 [SMILES: Cc1cccc(-c2ccc(-n3ncc4cc(F)c(O)cc43)cc2)c1] | 5-Fluoro-1-(3′-methyl-[1,1′-biphenyl]-4-yl)- 1H-indazol-6-ol |
| 13.11 | 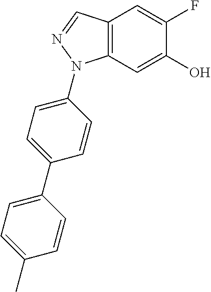 [SMILES: Cc1ccc(-c2ccc(-n3ncc4cc(F)c(O)cc43)cc2)cc1] | 5-Fluoro-1-(4′-methyl-[1,1′-biphenyl]-4-yl)- 1H-indazol-6-ol |
| 13.12 | 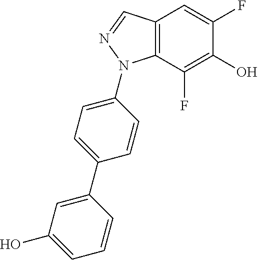 [SMILES: Oc1cccc(-c2ccc(-n3ncc4cc(F)c(O)c(F)c43)cc2)c1] | 5,7-Difluoro-1-(3′-hydroxy-[1,1′-biphenyl]- 4-yl)-1H-indazol-6-ol |
| 13.13 | 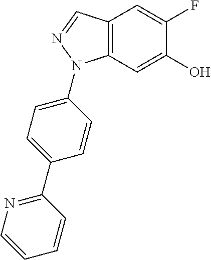 [SMILES: Oc1cc2c(cnn2-c2ccc(-c3ccccn3)cc2)cc1F] | 5-Fluoro-1-(4-(pyridin-2-yl)phenyl)-1H- indazol-6-ol |
| 13.14 | 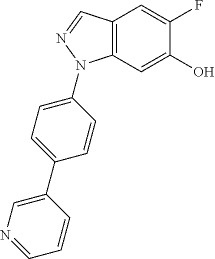 [SMILES: Oc1cc2c(cnn2-c2ccc(-c3cccnc3)cc2)cc1F] | 5-Fluoro-1-(4-(pyridin-3-yl)phenyl)-1H- indazol-6-ol |
| 13.15 | 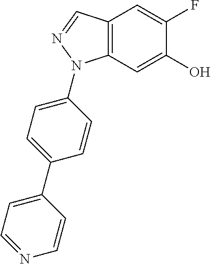 [SMILES: Oc1cc2c(cnn2-c2ccc(-c3ccncc3)cc2)cc1F] | 5-Fluoro-1-(4-(pyridin-4-yl)phenyl)-1H- indazol-6-ol |
| 13.16 | 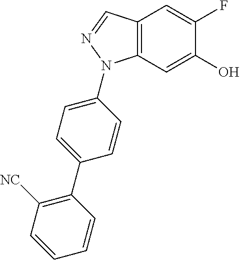 [SMILES: N#Cc1ccccc1-c1ccc(-n2ncc3cc(F)c(O)cc32)cc1] | 4′-(5-Fluoro-6-hydroxy-1H-indazol-1-yl)- [1,1′-biphenyl]-2-carbonitrile |
| 13.17 | 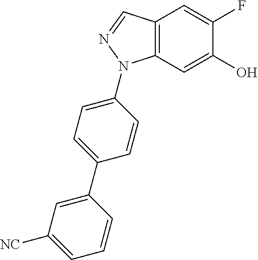 [SMILES: N#Cc1cccc(-c2ccc(-n3ncc4cc(F)c(O)cc43)cc2)c1] | 4′-(5-Fluoro-6-hydroxy-1H-indazol-1-yl)- [1,1′-biphenyl]-3-carbonitrile |
| 13.18 | 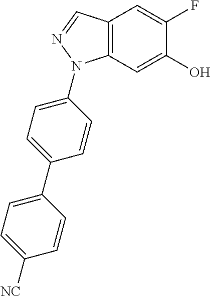 [SMILES: N#Cc1ccc(-c2ccc(-n3ncc4cc(F)c(O)cc43)cc2)cc1] | 4′-(5-Fluoro-6-hydroxy-1H-indazol-1-yl)- [1,1′-biphenyl]-4-carbonitrile |
| 13.19 | 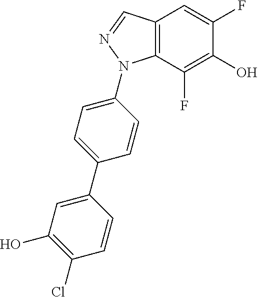 [SMILES: Oc1cc(-c2ccc(-n3ncc4cc(F)c(O)c(F)c43)cc2)ccc1Cl] | 1-(4′-Chloro-3′-hydroxy-[1,1′-biphenyl]-4- yl)-5,7-difluoro-1H-indazol-6-ol |
| 13.20 | 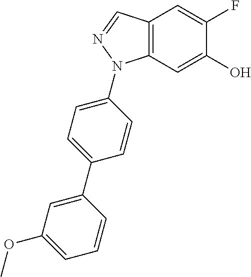 [SMILES: COc1cccc(-c2ccc(-n3ncc4cc(F)c(O)cc43)cc2)c1] | 5-Fluoro-1-(3′-methoxy-[1,1′-biphenyl]-4- yl)-1H-indazol-6-ol |
| 13.21 | 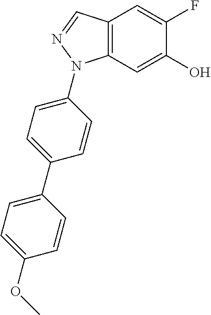 [SMILES: COc1ccc(-c2ccc(-n3ncc4cc(F)c(O)cc43)cc2)cc1] | 5-Fluoro-1-(4′-methoxy-[1,1′-biphenyl]-4- yl)-1H-indazol-6-ol |
| 13.22 | 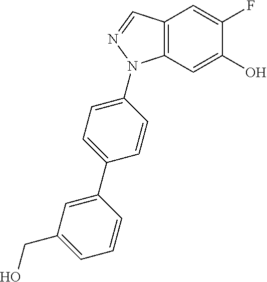 [SMILES: OCc1cccc(-c2ccc(-n3ncc4cc(F)c(O)cc43)cc2)c1] | 5-Fluoro-1-(3′-(hydroxymethyl)-[1,1′- biphenyl]-4-yl)-1H-indazol-6-ol |
| 13.23 | 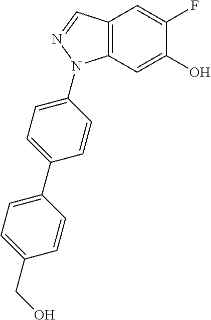 [SMILES: OCc1ccc(-c2ccc(-n3ncc4cc(F)c(O)cc43)cc2)cc1] | 5-Fluoro-1-(4′-(hydroxymethyl)-[1,1′- biphenyl]-4-yl)-1H-indazol-6-ol |
| 13.24 | 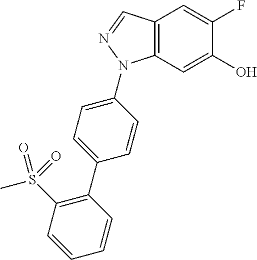 [SMILES: CS(=O)(=O)c1ccccc1-c1ccc(-n2ncc3cc(F)c(O)cc32)cc1] | 5-Fluoro-1-(2′-(methylsulfonyl)-[1,1′- biphenyl]-4-yl)-1H-indazol-6-ol |
| 13.25 | 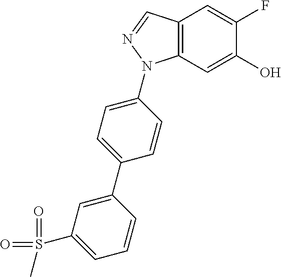 [SMILES: CS(=O)(=O)c1cccc(-c2ccc(-n3ncc4cc(F)c(O)cc43)cc2)c1] | 5-Fluoro-1-(3′-(methylsulfonyl)-[1,1′- biphenyl]-4-yl)-1H-indazol-6-ol |
| 13.26 | 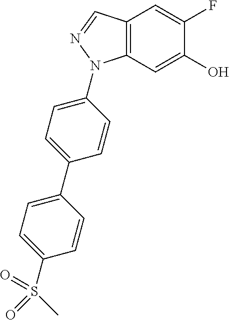 [SMILES: CS(=O)(=O)c1ccc(-c2ccc(-n3ncc4cc(F)c(O)cc43)cc2)cc1] | 5-Fluoro-1-(4′-(methylsulfonyl)-[1,1′- biphenyl]-4-yl)-1H-indazol-6-ol |
| 13.27 | 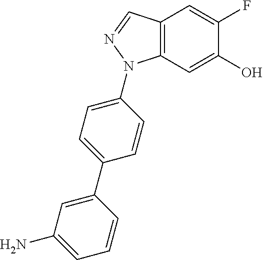 [SMILES: Nc1cccc(-c2ccc(-n3ncc4cc(F)c(O)cc43)cc2)c1] | 1-(3′-Amino-[1,1′-biphenyl]-4-yl)-5-fluoro- 1H-indazol-6-ol |
| 13.28 | 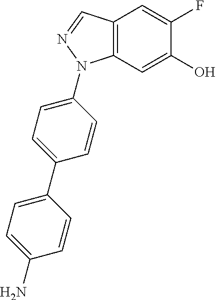 [SMILES: Nc1ccc(-c2ccc(-n3ncc4cc(F)c(O)cc43)cc2)cc1] | 1-(4′-Amino-[1,1′-biphenyl]-4-yl)-5-fluoro- 1H-indazol-6-ol |
| 13.29 | 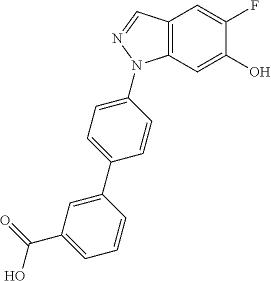 [SMILES: O=C(O)c1cccc(-c2ccc(-n3ncc4cc(F)c(O)cc43)cc2)c1] | 4′-(5-Fluoro-6-hydroxy-1H-indazol-1-yl)- [1,1′-biphenyl]-3-carboxylic acid |
| 13.30 | 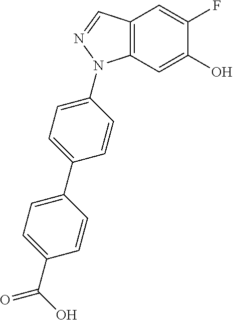 [SMILES: O=C(O)c1ccc(-c2ccc(-n3ncc4cc(F)c(O)cc43)cc2)cc1] | 4′-(5-Fluoro-6-hydroxy-1H-indazol-1-yl)- [1,1′-biphenyl]-4-carboxylic acid |
| 13.31 | 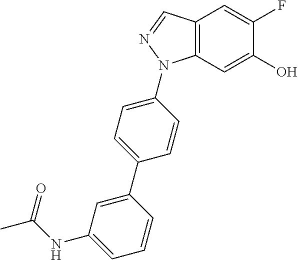 [SMILES: CC(=O)Nc1cccc(-c2ccc(-n3ncc4cc(F)c(O)cc43)cc2)c1] | N-(4′-(5-Fluoro-6-hydroxy-1H-indazol-1-yl)- [1,1′-biphenyl]-3-yl)acetamide |
| 13.32 | 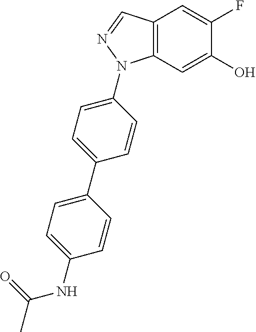 [SMILES: CC(=O)Nc1ccc(-c2ccc(-n3ncc4cc(F)c(O)cc43)cc2)cc1] | N-(4′-(5-Fluoro-6-hydroxy-1H-indazol-1-yl)- [1,1′-biphenyl]-4-yl)acetamide |
| 13.33 | 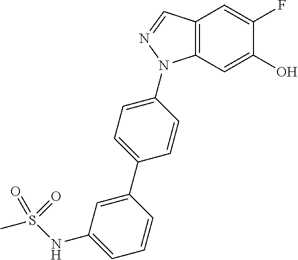 [SMILES: CS(=O)(=O)Nc1cccc(-c2ccc(-n3ncc4cc(F)c(O)cc43)cc2)c1] | N-(4′-(5-Fluoro-6-hydroxy-1H-indazol-1-yl)- [1,1′-biphenyl]-3-yl)methanesulfonamide |
| 13.34 | 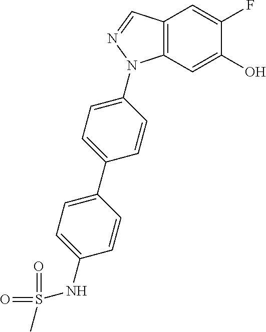 [SMILES: CS(=O)(=O)Nc1ccc(-c2ccc(-n3ncc4cc(F)c(O)cc43)cc2)cc1] | N-(4′-(5-Fluoro-6-hydroxy-1H-indazol-1-yl)- [1,1′-biphenyl]-4-yl)methanesulfonamide |
| 13.35 | 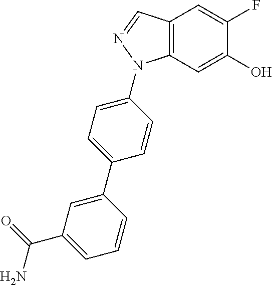 [SMILES: NC(=O)c1cccc(-c2ccc(-n3ncc4cc(F)c(O)cc43)cc2)c1] | 4′-(5-Fluoro-6-hydroxy-1H-indazol-1-yl)- [1,1′-biphenyl]-3-carboxamide |
| 13.36 | 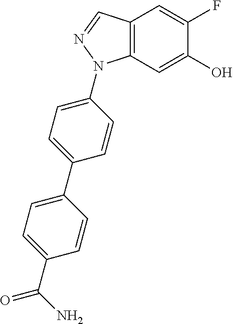 [SMILES: NC(=O)c1ccc(-c2ccc(-n3ncc4cc(F)c(O)cc43)cc2)cc1] | 4′-(5-Fluoro-6-hydroxy-1H-indazol-1-yl)- [1,1′-biphenyl]-4-carboxamide |
| 13.37 | 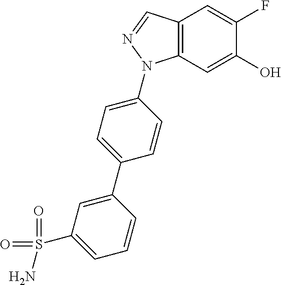 [SMILES: NS(=O)(=O)c1cccc(-c2ccc(-n3ncc4cc(F)c(O)cc43)cc2)c1] | 4′-(5-Fluoro-6-hydroxy-1H-indazol-1-yl)- [1,1′-biphenyl]-3-sulfonamide |
| 13.38 | 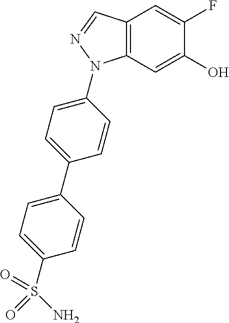 [SMILES: NS(=O)(=O)c1ccc(-c2ccc(-n3ncc4cc(F)c(O)cc43)cc2)cc1] | 4′-(5-Fluoro-6-hydroxy-1H-indazol-1-yl)- [1,1′-biphenyl]-4-sulfonamide |
| 13.39 | 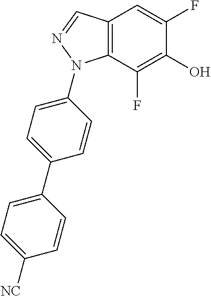 [SMILES: N#Cc1ccc(-c2ccc(-n3ncc4cc(F)c(O)c(F)c43)cc2)cc1] | 4′-(5,7-Difluoro-6-hydroxy-1H-indazol-1- yl)-[1,1′-biphenyl]-4-carbonitrile |
| 13.40 | 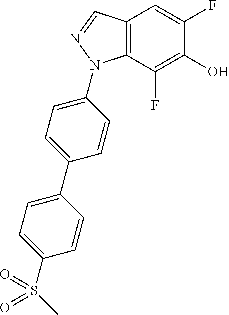 [SMILES: CS(=O)(=O)c1ccc(-c2ccc(-n3ncc4cc(F)c(O)c(F)c43)cc2)cc1] | 5,7-Difluoro-1-(4′-(methylsulfonyl)-[1,1′- biphenyl]-4-yl)-1H-indazol-6-ol |
| 14 | 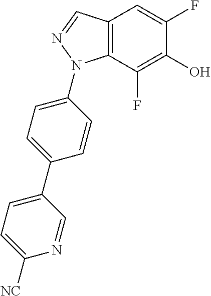 [SMILES: N#Cc1ccc(-c2ccc(-n3ncc4cc(F)c(O)c(F)c43)cc2)cn1] | 5-(4-(5,7-Difluoro-6-hydroxy-1H-indazol-1- yl)phenyl)picolinonitrile |
| 14.1 | 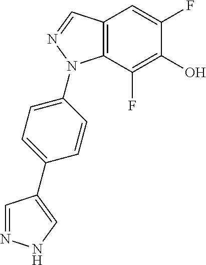 [SMILES: Oc1c(F)cc2cnn(-c3ccc(-c4cn[nH]c4)cc3)c2c1F] | 1-(4-(1H-Pyrazol-4-yl)phenyl)-5,7-difluoro- 1H-indazol-6-ol |
| 14.2 | 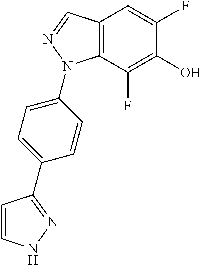 [SMILES: Oc1c(F)cc2cnn(-c3ccc(-c4cc[nH]n4)cc3)c2c1F] | 1-(4-(1H-Pyrazol-3-yl)phenyl)-5,7-difluoro- 1H-indazol-6-ol |
| 14.3 | 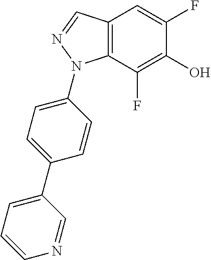 [SMILES: Oc1c(F)cc2cnn(-c3ccc(-c4cccnc4)cc3)c2c1F] | 5,7-Difluoro-1-(4-(pyridin-3-yl)phenyl)-1H- indazol-6-ol |
| 14.4 | 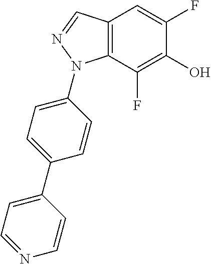 [SMILES: Oc1c(F)cc2cnn(-c3ccc(-c4ccncc4)cc3)c2c1F] | 5,7-Difluoro-1-(4-(pyridin-4-yl)phenyl)-1H- indazol-6-ol |
| 15 | 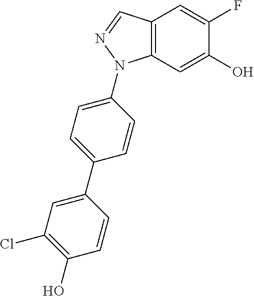 [SMILES: Oc1cc2c(cnn2-c2ccc(-c3ccc(O)c(Cl)c3)cc2)cc1F] | 1-(3′-Chloro-4′-hydroxy-[1,1′-biphenyl]-4- yl)-5-fluoro-1H-indazol-6-ol |
| 15.1 | 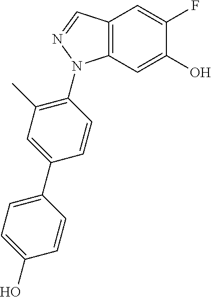 [SMILES: Cc1cc(-c2ccc(O)cc2)ccc1-n1ncc2cc(F)c(O)cc21] | 5-Fluoro-1-(4′-hydroxy-3-methyl-[1,1′- biphenyl]-4-yl)-1H-indazol-6-ol |
| 15.2 | 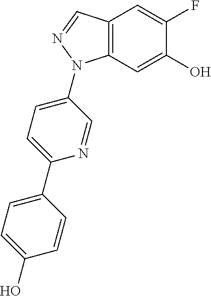 [SMILES: Oc1ccc(-c2ccc(-n3ncc4cc(F)c(O)cc43)cn2)cc1] | 5-Fluoro-1-(6-(4-hydroxyphenyl)pyridin-3- yl)-1H-indazol-6-ol |
| 15.3 | 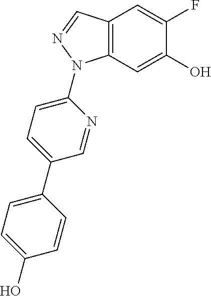 [SMILES: Oc1ccc(-c2ccc(-n3ncc4cc(F)c(O)cc43)nc2)cc1] | 5-Fluoro-1-(5-(4-hydroxyphenyl)pyridin-2- yl)-1H-indazol-6-ol |
| 15.4 | 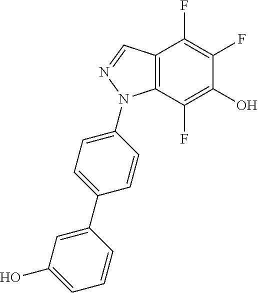 [SMILES: Oc1cccc(-c2ccc(-n3ncc4c(F)c(F)c(O)c(F)c43)cc2)c1] | 4,5,7-Trifluoro-1-(3′-hydroxy-[1,1′- biphenyl]-4-yl)-1H-indazol-6-ol |
| 16 | 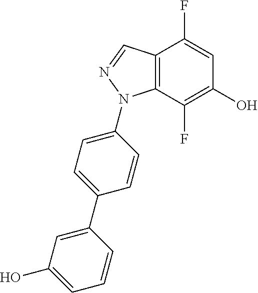 [SMILES: Oc1cccc(-c2ccc(-n3ncc4c(F)cc(O)c(F)c43)cc2)c1] | 4,7-Difluoro-1-(3′-hydroxy-[1,1′-biphenyl]- 4-yl)-1H-indazol-6-ol |
| 17 | 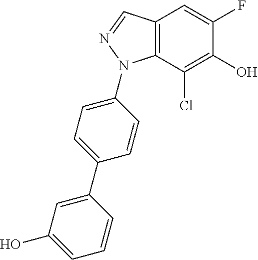 [SMILES: Oc1cccc(-c2ccc(-n3ncc4cc(F)c(O)c(Cl)c43)cc2)c1] | 7-Chloro-5-fluoro-1-(3′-hydroxy-[1,1′- biphenyl]-4-yl)-1H-indazol-6-ol |
| 18 | 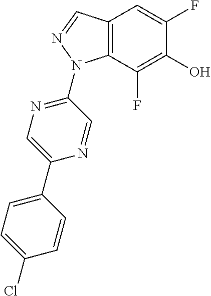 [SMILES: Oc1c(F)cc2cnn(-c3cnc(-c4ccc(Cl)cc4)cn3)c2c1F] | 1-(5-(4-Chlorophenyl)pyrazin-2-yl)-5,7- difluoro-1H-indazol-6-ol |
| 18.1 | 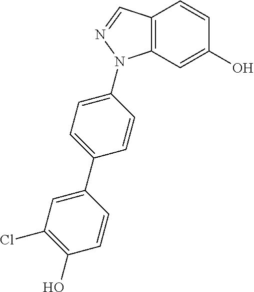 [SMILES: Oc1ccc2cnn(-c3ccc(-c4ccc(O)c(Cl)c4)cc3)c2c1] | 1-(3′-Chloro-4′-hydroxy-[1,1′-biphenyl]-4- yl)-1H-indazol-6-ol |
| 18.2 | 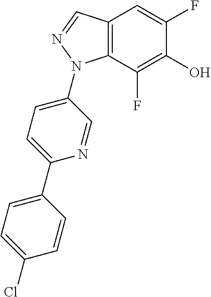 [SMILES: Oc1c(F)cc2cnn(-c3ccc(-c4ccc(Cl)cc4)nc3)c2c1F] | 1-(6-(4-Chlorophenyl)pyridin-3-yl)-5,7- difluoro-1H-indazol-6-ol |
| 18.3 | 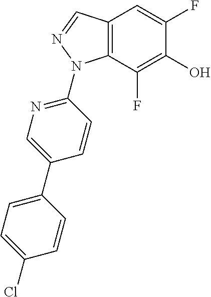 [SMILES: Oc1c(F)cc2cnn(-c3ccc(-c4ccc(Cl)cc4)cn3)c2c1F] | 1-(5-(4-Chlorophenyl)pyridin-2-yl)-5,7- difluoro-1H-indazol-6-ol |
| 18.4 | 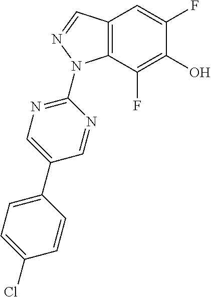 [SMILES: Oc1c(F)cc2cnn(-c3ncc(-c4ccc(Cl)cc4)cn3)c2c1F] | 1-(5-(4-Chlorophenyl)pyrimidin-2-yl)-5,7- difluoro-1H-indazol-6-ol |
| 19 | 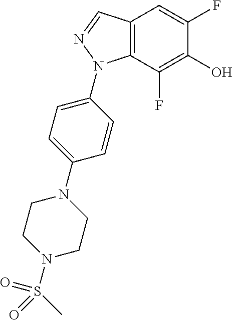 [SMILES: CS(=O)(=O)N1CCN(c2ccc(-n3ncc4cc(F)c(O)c(F)c43)cc2)CC1] | 5,7-Difluoro-1-(4-(4- (methylsulfonyl)piperazin-1-yl)phenyl)-1H- indazol-6-ol |
| 19.1 | 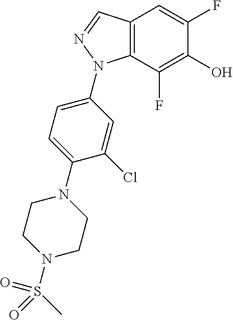 [SMILES: CS(=O)(=O)N1CCN(c2ccc(-n3ncc4cc(F)c(O)c(F)c43)cc2Cl)CC1] | 1-(3-Chloro-4-(4-(methylsulfonyl)piperazin- 1-yl)phenyl)-5,7-difluoro-1H-indazol-6-ol |
| 20 | 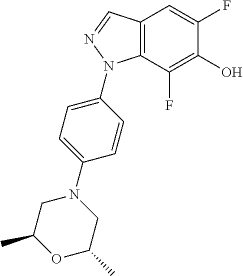 [SMILES: C[C@H]1CN(c2ccc(-n3ncc4cc(F)c(O)c(F)c43)cc2)C[C@H](C)O1] | 1-(4-(trans-2,6- Dimethylmorpholino)phenyl)-5,7-difluoro- 1H-indazol-6-ol |
| 20.1 | 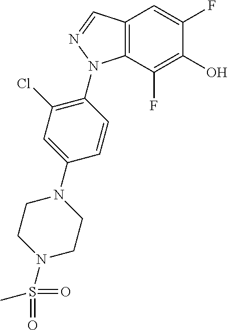 [SMILES: CS(=O)(=O)N1CCN(c2ccc(-n3ncc4cc(F)c(O)c(F)c43)c(Cl)c2)CC1] | 1-(2-Chloro-4-(4-(methylsulfonyl)piperazin- 1-yl)phenyl)-5,7-difluoro-1H-indazol-6-ol |
| 20.2 | 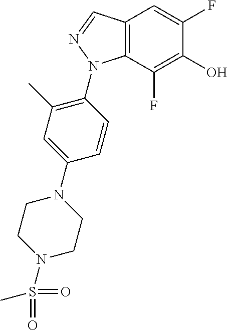 [SMILES: Cc1cc(N2CCN(S(C)(=O)=O)CC2)ccc1-n1ncc2cc(F)c(O)c(F)c21] | 5,7-Difluoro-1-(2-methyl-4-(4- (methylsulfonyl)piperazin-1-yl)phenyl)-1H- indazol-6-ol |
| 20.3 | 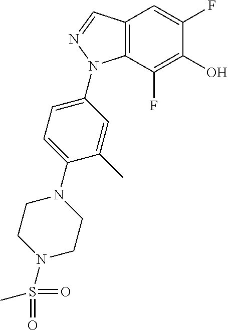 [SMILES: Cc1cc(-n2ncc3cc(F)c(O)c(F)c32)ccc1N1CCN(S(C)(=O)=O)CC1] | 5,7-Difluoro-1-(3-methyl-4-(4- (methylsulfonyl)piperazin-1-yl)phenyl)-1H- indazol-6-ol |
| 20.4 | 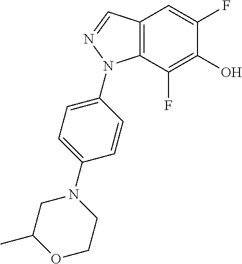 [SMILES: CC1CN(c2ccc(-n3ncc4cc(F)c(O)c(F)c43)cc2)CCO1] | 5,7-Difluoro-1-(4-(2- methylmorpholino)phenyl)-1H-indazol-6-ol |
| 20.5 | 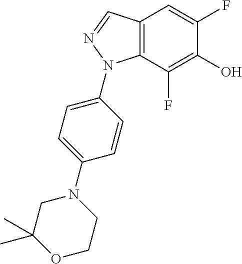 [SMILES: CC1(C)CN(c2ccc(-n3ncc4cc(F)c(O)c(F)c43)cc2)CCO1] | 1-(4-(2,2-Dimethylmorpholino)phenyl)-5,7- difluoro-1H-indazol-6-ol |
| 20.6 | 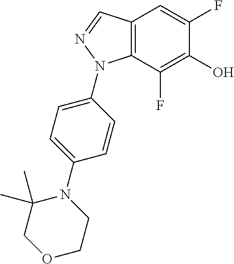 [SMILES: CC1(C)COCCN1c1ccc(-n2ncc3cc(F)c(O)c(F)c32)cc1] | 1-(4-(3,3-Dimethylmorpholino)phenyl)-5,7- difluoro-1H-indazol-6-ol |
| 20.7 | 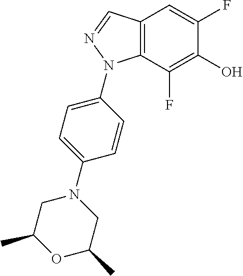 [SMILES: C[C@@H]1CN(c2ccc(-n3ncc4cc(F)c(O)c(F)c43)cc2)C[C@H](C)O1] | 1-(4-(cis-2,6-Dimethylmorpholino)phenyl)- 5,7-difluoro-1H-indazol-6-ol |
| 20.8 | 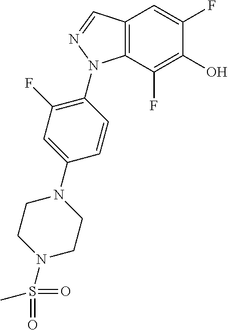 [SMILES: CS(=O)(=O)N1CCN(c2ccc(-n3ncc4cc(F)c(O)c(F)c43)c(F)c2)CC1] | 5,7-Difluoro-1-(2-fluoro-4-(4- (methylsulfonyl)piperazin-1-yl)phenyl)-1H- indazol-6-ol |
| 20.9 | 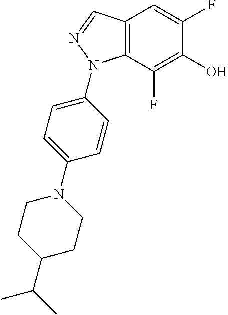 [SMILES: CC(C)C1CCN(c2ccc(-n3ncc4cc(F)c(O)c(F)c43)cc2)CC1] | 5,7-Difluoro-1-(4-(4-isopropylpiperidin-1- yl)phenyl)-1H-indazol-6-ol |
| 20.10 | 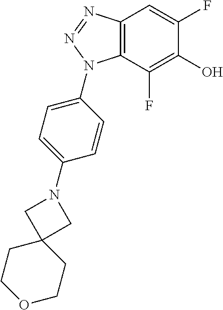 [SMILES: Oc1c(F)cc2nnn(-c3ccc(N4CC5(CCOCC5)C4)cc3)c2c1F] | 1-(4-(7-Oxa-2-azaspiro[3.5]nonan-2- yl)phenyl)-5,7-difluoro-1H- benzo[d][1,2,3]triazol-6-ol |
| 20.11 | 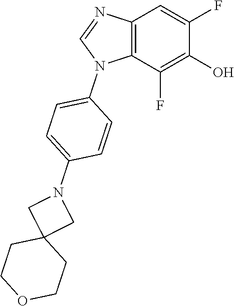 [SMILES: Oc1c(F)cc2ncn(-c3ccc(N4CC5(CCOCC5)C4)cc3)c2c1F] | 1-(4-(7-Oxa-2-azaspiro[3.5]nonan-2- yl)phenyl)-5,7-difluoro-1H- benzo[d]imidazol-6-ol |
| 20.12 | 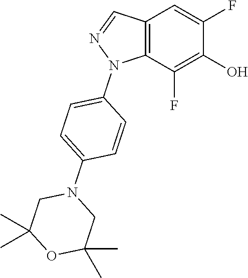 [SMILES: CC1(C)CN(c2ccc(-n3ncc4cc(F)c(O)c(F)c43)cc2)CC(C)(C)O1] | 5,7-Difluoro-1-(4-(2,2,6,6- tetramethylmorpholino)phenyl)-1H-indazol- 6-ol |
| 20.13 | 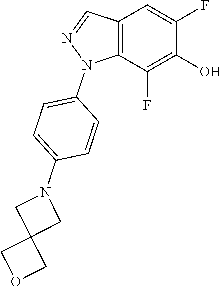 [SMILES: Oc1c(F)cc2cnn(-c3ccc(N4CC5(COC5)C4)cc3)c2c1F] | 1-(4-(2-Oxa-6-azaspiro[3.3]heptan-6- yl)phenyl)-5,7-difluoro-1H-indazol-6-ol |
| 20.14 | 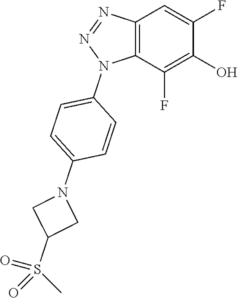 [SMILES: CS(=O)(=O)C1CN(c2ccc(-n3nnc4cc(F)c(O)c(F)c43)cc2)C1] | 5,7-Difluoro-1-(4-(3- (methylsulfonyl)azetidin-1-yl)phenyl)-1H- benzo[d][1,2,3]triazol-6-ol |
| 20.15 | 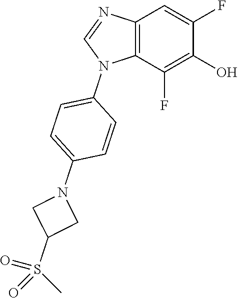 [SMILES: CS(=O)(=O)C1CN(c2ccc(-n3cnc4cc(F)c(O)c(F)c43)cc2)C1] | 5,7-Difluoro-1-(4-(3- (methylsulfonyl)azetidin-1-yl)phenyl)-1H- benzo[d]imidazol-6-ol |
| 20.16 | 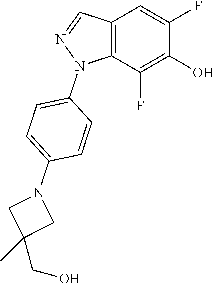 [SMILES: CC1(CO)CN(c2ccc(-n3ncc4cc(F)c(O)c(F)c43)cc2)C1] | 5,7-Difluoro-1-(4-(3-(hydroxymethyl)-3- methylazetidin-1-yl)phenyl)-1H-indazol-6-ol |
| 20.17 | 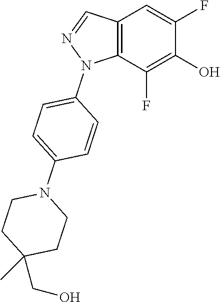 [SMILES: CC1(CO)CCN(c2ccc(-n3ncc4cc(F)c(O)c(F)c43)cc2)CC1] | 5,7-Difluoro-1-(4-(4-(hydroxymethyl)-4- methylpiperidin-1-yl)phenyl)-1H-indazol-6- ol |
| 20.18 | 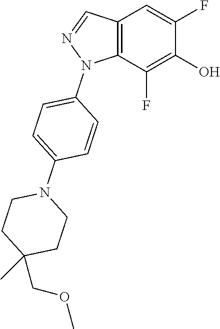 [SMILES: COCC1(C)CCN(c2ccc(-n3ncc4cc(F)c(O)c(F)c43)cc2)CC1] | 5,7-Difluoro-1-(4-(4-(methoxymethyl)-4- methylpiperidin-1-yl)phenyl)-1H-indazol-6- ol |
| 20.19 | 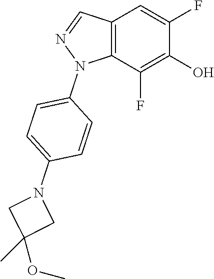 [SMILES: COC1(C)CN(c2ccc(-n3ncc4cc(F)c(O)c(F)c43)cc2)C1] | 5,7-Difluoro-1-(4-(3-methoxy-3- methylazetidin-1-yl)phenyl)-1H-indazol-6-ol |
| 20.20 | 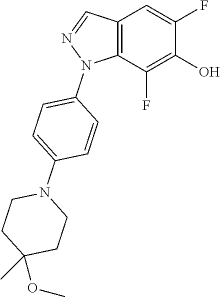 [SMILES: COC1(C)CCN(c2ccc(-n3ncc4cc(F)c(O)c(F)c43)cc2)CC1] | 5,7-Difluoro-1-(4-(4-methoxy-4- methylpiperidin-1-yl)phenyl)-1H-indazol-6- ol |
| 20.21 | 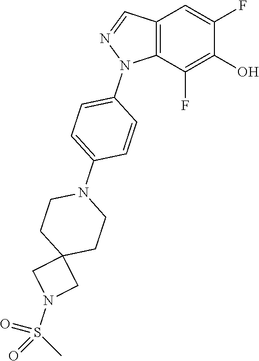 [SMILES: CS(=O)(=O)N1CC2(CCN(c3ccc(-n4ncc5cc(F)c(O)c(F)c54)cc3)CC2)C1] | 5,7-Difluoro-1-(4-(2-(methylsulfonyl)-2,7- diazaspiro[3.5]nonan-7-yl)phenyl)-1H- indazol-6-ol |
| 20.22 | 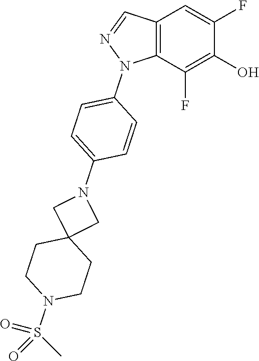 [SMILES: CS(=O)(=O)N1CCC2(CC1)CN(c1ccc(-n3ncc4cc(F)c(O)c(F)c43)cc1)C2] | 5,7-Difluoro-1-(4-(7-(methylsulfonyl)-2,7- diazaspiro[3.5]nonan-2-yl)phenyl)-1H- indazol-6-ol |
| 20.23 | 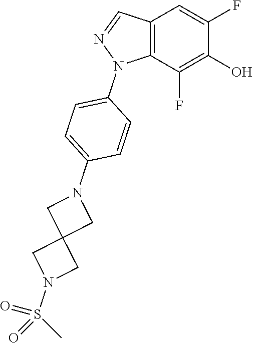 [SMILES: CS(=O)(=O)N1CC2(CN(c3ccc(-n4ncc5cc(F)c(O)c(F)c54)cc3)C2)C1] | 5,7-Difluoro-1-(4-(6-(methylsulfonyl)-2,6- diazaspiro[3.3]heptan-2-yl)phenyl)-1H- indazol-6-ol |
| 21 | 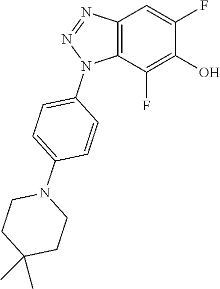 [SMILES: CC1(C)CCN(c2ccc(-n3nnc4cc(F)c(O)c(F)c43)cc2)CC1] | 1-(4-(4,4-Dimethylpiperidin-1-yl)phenyl)- 5,7-difluoro-1H-benzo[d][1,2,3]triazol-6-ol |
| 21.1 | 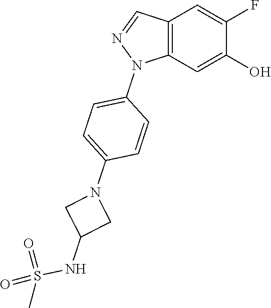 [SMILES: CS(=O)(=O)NC1CN(c2ccc(-n3ncc4cc(F)c(O)cc43)cc2)C1] | N-(1-(4-(5-Fluoro-6-hydroxy-1H-indazol-1- yl)phenyl)azetidin-3-yl)methanesulfonamide |
| 21.2 | 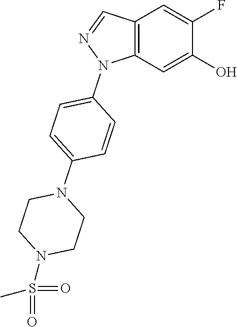 [SMILES: CS(=O)(=O)N1CCN(c2ccc(-n3ncc4cc(F)c(O)cc43)cc2)CC1] | 5-Fluoro-1-(4-(4-(methylsulfonyl)piperazin- 1-yl)phenyl)-1H-indazol-6-ol |
| 21.3 | 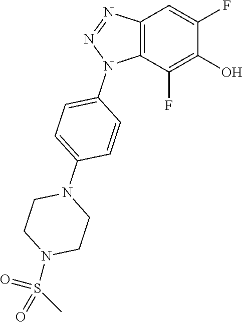 [SMILES: CS(=O)(=O)N1CCN(c2ccc(-n3nnc4cc(F)c(O)c(F)c43)cc2)CC1] | 5,7-Difluoro-1-(4-(4- (methylsulfonyl)piperazin-1-yl)phenyl)-1H- benzo[d][1,2,3]triazol-6-ol |
| 21.4 | 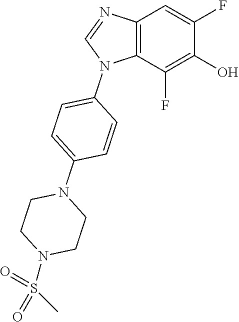 [SMILES: CS(=O)(=O)N1CCN(c2ccc(-n3cnc4cc(F)c(O)c(F)c43)cc2)CC1] | 5,7-Difluoro-1-(4-(4- (methylsulfonyl)piperazin-1-yl)phenyl)-1H- benzo[d]imidazol-6-ol |
| 21.5 | 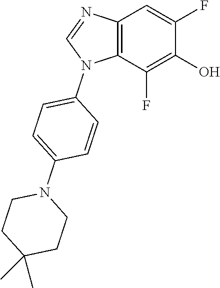 [SMILES: CC1(C)CCN(c2ccc(-n3cnc4cc(F)c(O)c(F)c43)cc2)CC1] | 1-(4-(4,4-Dimethylpiperidin-1-yl)phenyl)- 5,7-difluoro-1H-benzo[d]imidazol-6-ol |
| 21.6 | 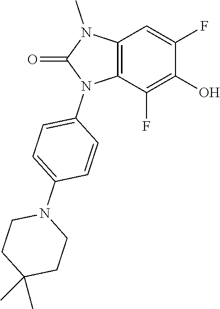 [SMILES: Cn1c(=O)n(-c2ccc(N3CCC(C)(C)CC3)cc2)c2c(F)c(O)c(F)cc21] | 3-(4-(4,4-Dimethylpiperidin-1-yl)phenyl)- 4,6-difluoro-5-hydroxy-1-methyl-1H- benzo[d]imidazol-2(3H)-one |
| 21.7 | 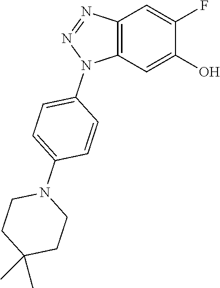 [SMILES: CC1(C)CCN(c2ccc(-n3nnc4cc(F)c(O)cc43)cc2)CC1] | 1-(4-(4,4-Dimethylpiperidin-1-yl)phenyl)-5- fluoro-1H-benzo[d][1,2,3]triazol-6-ol |
| 21.8 | 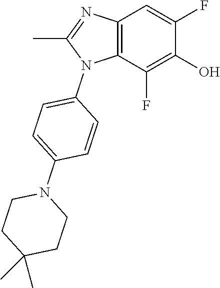 [SMILES: Cc1nc2cc(F)c(O)c(F)c2n1-c1ccc(N2CCC(C)(C)CC2)cc1] | 1-(4-(4,4-Dimethylpiperidin-1-yl)phenyl)- 5,7-difluoro-2-methyl-1H-benzo[d]imidazol- 6-ol |
| 21.9 | 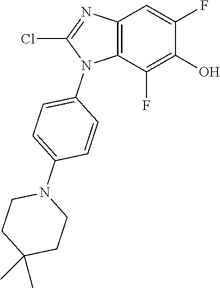 [SMILES: CC1(C)CCN(c2ccc(-n3c(Cl)nc4cc(F)c(O)c(F)c43)cc2)CC1] | 2-Chloro-1-(4-(4,4-dimethylpiperidin-1- yl)phenyl)-5,7-difluoro-1H- benzo[d]imidazol-6-ol |
| 21.10 | 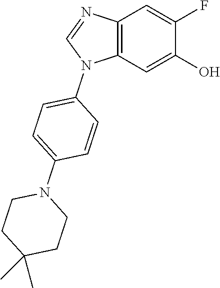 [SMILES: CC1(C)CCN(c2ccc(-n3cnc4cc(F)c(O)cc43)cc2)CC1] | 1-(4-(4,4-Dimethylpiperidin-1-yl)phenyl)-5- fluoro-1H-benzo[d]imidazol-6-ol |
| 21.11 | 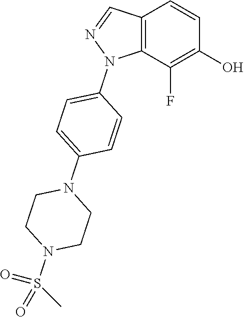 [SMILES: CS(=O)(=O)N1CCN(c2ccc(-n3ncc4ccc(O)c(F)c43)cc2)CC1] | 7-Fluoro-1-(4-(4-(methylsulfonyl)piperazin- 1-yl)phenyl)-1H-indazol-6-ol |
| 21.12 | 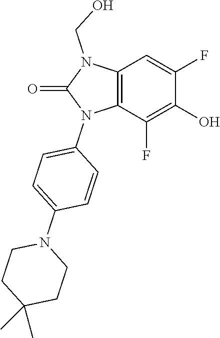 [SMILES: CC1(C)CCN(c2ccc(-n3c(=O)n(CO)c4cc(F)c(O)c(F)c43)cc2)CC1] | 3-(4-(4,4-Dimethylpiperidin-1-yl)phenyl)- 4,6-difluoro-5-hydroxy-1-(hydroxymethyl)- 1H-benzo[d]imidazol-2(3H)-one |
| 21.13 | 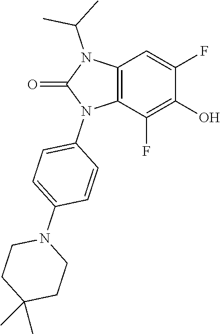 [SMILES: CC(C)n1c(=O)n(-c2ccc(N3CCC(C)(C)CC3)cc2)c2c(F)c(O)c(F)cc21] | 3-(4-(4,4-Dimethylpiperidin-1-yl)phenyl)- 4,6-difluoro-5-hydroxy-1-isopropyl-1H- benzo[d]imidazol-2(3H)-one |
| 22 | 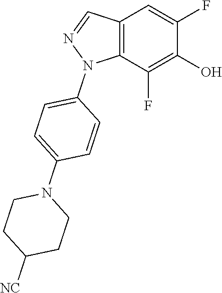 [SMILES: N#CC1CCN(c2ccc(-n3ncc4cc(F)c(O)c(F)c43)cc2)CC1] | 1-(4-(5,7-Difluoro-6-hydroxy-1H-indazol-1- yl)phenyl)piperidine-4-carbonitrile |
| 22.1 | 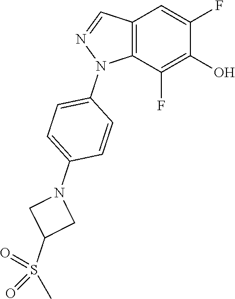 [SMILES: CS(=O)(=O)C1CN(c2ccc(-n3ncc4cc(F)c(O)c(F)c43)cc2)C1] | 5,7-Difluoro-1-(4-(3- (methylsulfonyl)azetidin-1-yl)phenyl)-1H- indazol-6-ol |
| 22.2 | 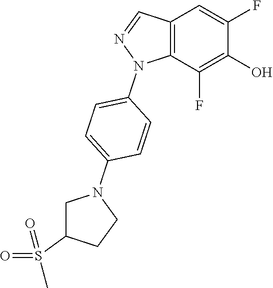 [SMILES: CS(=O)(=O)C1CCN(c2ccc(-n3ncc4cc(F)c(O)c(F)c43)cc2)C1] | 5,7-Difluoro-1-(4-(3- (methylsulfonyl)pyrrolidin-1-yl)phenyl)-1H- indazol-6-ol |
| 22.3 | 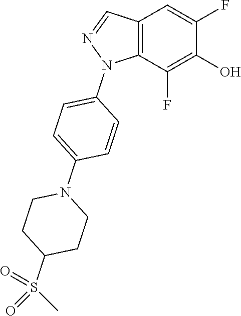 [SMILES: CS(=O)(=O)C1CCN(c2ccc(-n3ncc4cc(F)c(O)c(F)c43)cc2)CC1] | 5,7-Difluoro-1-(4-(4- (methylsulfonyl)piperidin-1-yl)phenyl)-1H- indazol-6-ol |
| 22.4 | 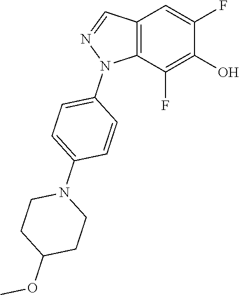 [SMILES: COC1CCN(c2ccc(-n3ncc4cc(F)c(O)c(F)c43)cc2)CC1] | 5,7-Difluoro-1-(4-(4-methoxypiperidin-1- yl)phenyl)-1H-indazol-6-ol |
| 22.5 | 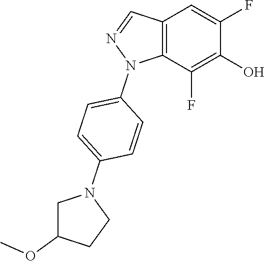 [SMILES: COC1CCN(c2ccc(-n3ncc4cc(F)c(O)c(F)c43)cc2)C1] | 5,7-Difluoro-1-(4-(3-methoxypyrrolidin-1- yl)phenyl)-1H-indazol-6-ol |
| 23 | 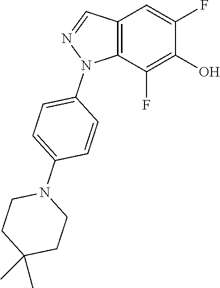 [SMILES: CC1(C)CCN(c2ccc(-n3ncc4cc(F)c(O)c(F)c43)cc2)CC1] | 1-(4-(4,4-Dimethylpiperidin-1-yl)phenyl)- 5,7-difluoro-1H-indazol-6-ol |
| 23.1 | 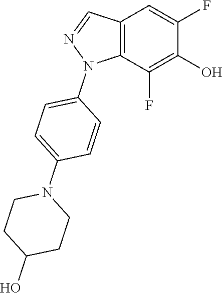 [SMILES: Oc1c(F)cc2cnn(-c3ccc(N4CCC(O)CC4)cc3)c2c1F] | 5,7-Difluoro-1-(4-(4-hydroxypiperidin-1- yl)phenyl)-1H-indazol-6-ol |
| 23.2 | 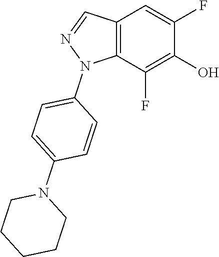 [SMILES: Oc1c(F)cc2cnn(-c3ccc(N4CCCCC4)cc3)c2c1F] | 5,7-Difluoro-1-(4-(piperidin-1-yl)phenyl)- 1H-indazol-6-ol |
| 23.3 | 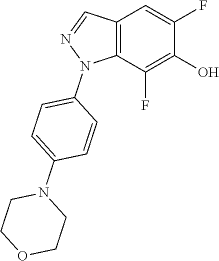 [SMILES: Oc1c(F)cc2cnn(-c3ccc(N4CCOCC4)cc3)c2c1F] | 5,7-Difluoro-1-(4-morpholinophenyl)-1H- indazol-6-ol |
| 23.4 | 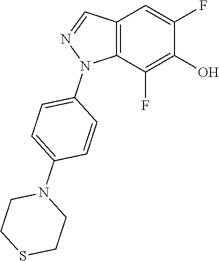 [SMILES: Oc1c(F)cc2cnn(-c3ccc(N4CCSCC4)cc3)c2c1F] | 5,7-Difluoro-1-(4-thiomorpholinophenyl)- 1H-indazol-6-ol |
| 23.5 | 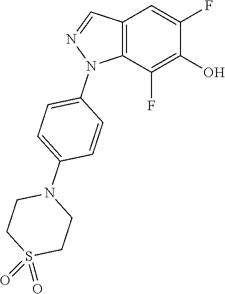 [SMILES: O=S1(=O)CCN(c2ccc(-n3ncc4cc(F)c(O)c(F)c43)cc2)CC1] | 4-(4-(5,7-Difluoro-6-hydroxy-1H-indazol-1- yl)phenyl)thiomorpholine 1,1-dioxide |
| 23.6 | 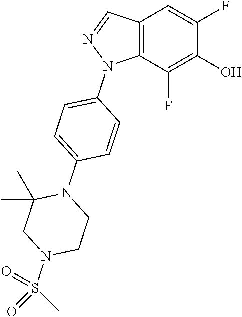 [SMILES: CC1(C)CN(S(C)(=O)=O)CCN1c1ccc(-n2ncc3cc(F)c(O)c(F)c32)cc1] | 1-(4-(2,2-Dimethyl-4- (methylsulfonyl)piperazin-1-yl)phenyl)-5,7- difluoro-1H-indazol-6-ol |
| 23.7 | 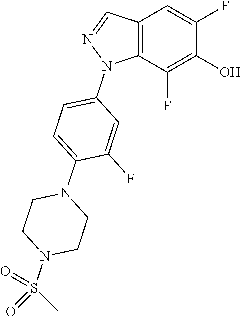 [SMILES: CS(=O)(=O)N1CCN(c2ccc(-n3ncc4cc(F)c(O)c(F)c43)cc2F)CC1] | 5,7-Difluoro-1-(3-fluoro-4-(4- (methylsulfonyl)piperazin-1-yl)phenyl)-1H- indazol-6-ol |
| 23.8 | 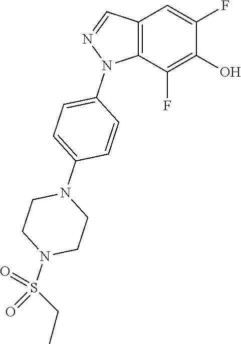 [SMILES: CCS(=O)(=O)N1CCN(c2ccc(-n3ncc4cc(F)c(O)c(F)c43)cc2)CC1] | 1-(4-(4-(Ethylsulfonyl)piperazin-1- yl)phenyl)-5,7-difluoro-1H-indazol-6-ol |
| 23.9 | 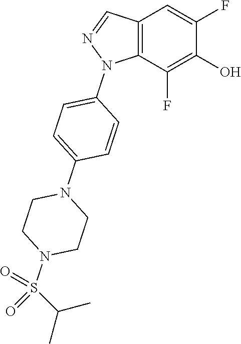 [SMILES: CC(C)S(=O)(=O)N1CCN(c2ccc(-n3ncc4cc(F)c(O)c(F)c43)cc2)CC1] | 5,7-Difluoro-1-(4-(4- (isopropylsulfonyl)piperazin-1-yl)phenyl)- 1H-indazol-6-ol |
| 23.10 | 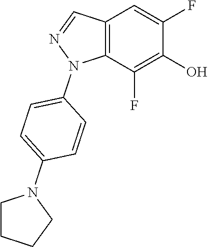 [SMILES: Oc1c(F)cc2cnn(-c3ccc(N4CCCC4)cc3)c2c1F] | 5,7-Difluoro-1-(4-(pyrrolidin-1-yl)phenyl)- 1H-indazol-6-ol |
| 23.11 | 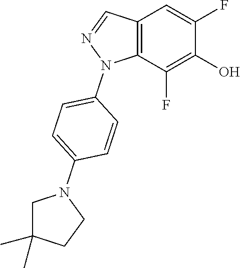 [SMILES: CC1(C)CCN(c2ccc(-n3ncc4cc(F)c(O)c(F)c43)cc2)C1] | 1-(4-(3,3-Dimethylpyrrolidin-1-yl)phenyl)- 5,7-difluoro-1H-indazol-6-ol |
| 23.12 | 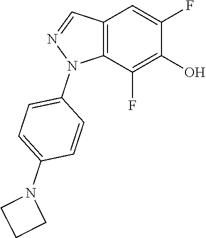 [SMILES: Oc1c(F)cc2cnn(-c3ccc(N4CCC4)cc3)c2c1F] | 1-(4-(Azetidin-1-yl)phenyl)-5,7-difluoro-1H- indazol-6-ol |
| 23.13 | 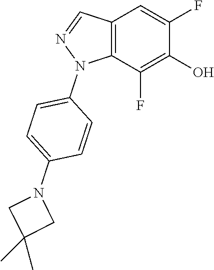 [SMILES: CC1(C)CN(c2ccc(-n3ncc4cc(F)c(O)c(F)c43)cc2)C1] | 1-(4-(3,3-Dimethylazetidin-1-yl)phenyl)-5,7- difluoro-1H-indazol-6-ol |
| 23.14 | 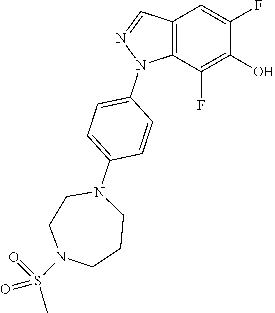 [SMILES: CS(=O)(=O)N1CCCN(c2ccc(-n3ncc4cc(F)c(O)c(F)c43)cc2)CC1] | 5,7-Difluoro-1-(4-(4-(methylsulfonyl)-1,4- diazepan-1-yl)phenyl)-1H-indazol-6-ol |
| 23.15 | 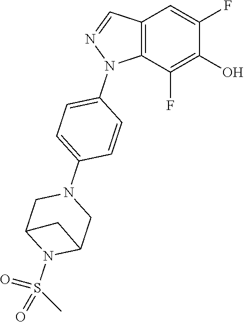 [SMILES: CS(=O)(=O)N1C2CC1CN(c1ccc(-n3ncc4cc(F)c(O)c(F)c43)cc1)C2] | 5,7-Difluoro-1-(4-(6-(methylsulfonyl)-3,6- diazabicyclo[3.1.1]heptan-3-yl)phenyl)-1H- indazol-6-ol |
| 23.16 | 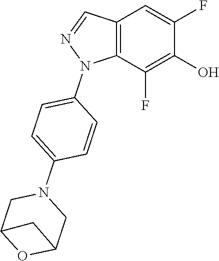 [SMILES: Oc1c(F)cc2cnn(-c3ccc(N4CC5CC(C4)O5)cc3)c2c1F] | 1-(4-(6-Oxa-3-azabicyclo[3.1.1]heptan-3- yl)phenyl)-5,7-difluoro-1H-indazol-6-ol |
| 23.17 | 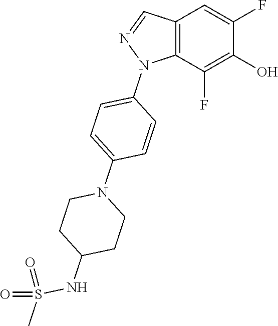 [SMILES: CS(=O)(=O)NC1CCN(c2ccc(-n3ncc4cc(F)c(O)c(F)c43)cc2)CC1] | N-(1-(4-(5,7-Difluoro-6-hydroxy-1H- indazol-1-yl)phenyl)piperidin-4- yl)methanesulfonamide |
| 23.18 | 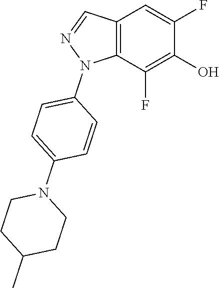 [SMILES: CC1CCN(c2ccc(-n3ncc4cc(F)c(O)c(F)c43)cc2)CC1] | 5,7-Difluoro-1-(4-(4-methylpiperidin-1- yl)phenyl)-1H-indazol-6-ol |
| 23.19 | 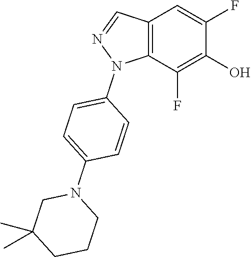 [SMILES: CC1(C)CCCN(c2ccc(-n3ncc4cc(F)c(O)c(F)c43)cc2)C1] | 1-(4-(3,3-Dimethylpiperidin-1-yl)phenyl)- 5,7-difluoro-1H-indazol-6-ol |
| 23.20 | 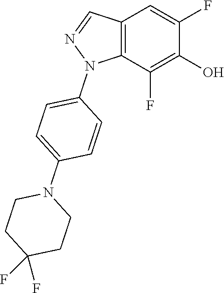 [SMILES: Oc1c(F)cc2cnn(-c3ccc(N4CCC(F)(F)CC4)cc3)c2c1F] | 1-(4-(4,4-Difluoropiperidin-1-yl)phenyl)- 5,7-difluoro-1H-indazol-6-ol |
| 23.21 | 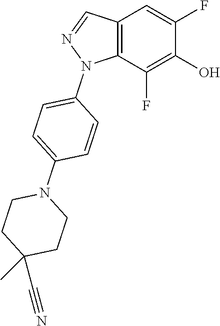 [SMILES: CC1(C#N)CCN(c2ccc(-n3ncc4cc(F)c(O)c(F)c43)cc2)CC1] | 1-(4-(5,7-Difluoro-6-hydroxy-1H-indazol-1- yl)phenyl)-4-methylpiperidine-4-carbonitrile |
| 23.22 | 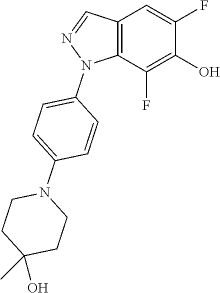 [SMILES: CC1(O)CCN(c2ccc(-n3ncc4cc(F)c(O)c(F)c43)cc2)CC1] | 5,7-Difluoro-1-(4-(4-hydroxy-4- methylpiperidin-1-yl)phenyl)-1H-indazol-6- ol |
| 23.23 | 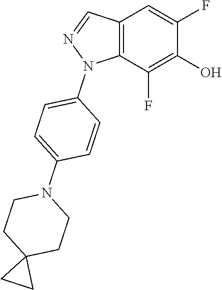 [SMILES: Oc1c(F)cc2cnn(-c3ccc(N4CCC5(CC4)CC5)cc3)c2c1F] | 1-(4-(6-Azaspiro[2.5]octan-6-yl)phenyl)-5,7- difluoro-1H-indazol-6-ol |
| 23.24 | 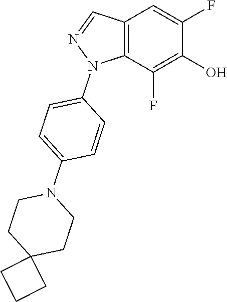 [SMILES: Oc1c(F)cc2cnn(-c3ccc(N4CCC5(CCC5)CC4)cc3)c2c1F] | 1-(4-(7-Azaspiro[3.5]nonan-7-yl)phenyl)- 5,7-difluoro-1H-indazol-6-ol |
| 23.25 | 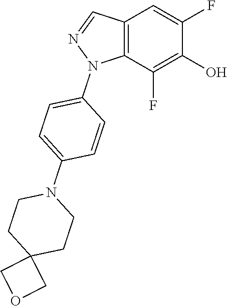 [SMILES: Oc1c(F)cc2cnn(-c3ccc(N4CCC5(CC4)COC5)cc3)c2c1F] | 1-(4-(2-Oxa-7-azaspiro[3.5]nonan-7- yl)phenyl)-5,7-difluoro-1H-indazol-6-ol |
| 23.26 | 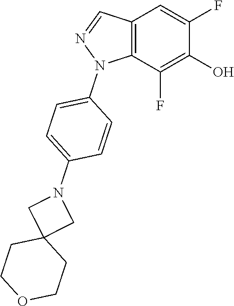 [SMILES: Oc1c(F)cc2cnn(-c3ccc(N4CC5(CCOCC5)C4)cc3)c2c1F] | 1-(4-(7-Oxa-2-azaspiro[3.5]nonan-2- yl)phenyl)-5,7-difluoro-1H-indazol-6-ol |
| 23.27 | 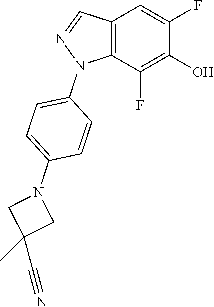 [SMILES: CC1(C#N)CN(c2ccc(-n3ncc4cc(F)c(O)c(F)c43)cc2)C1] | 1-(4-(5,7-Difluoro-6-hydroxy-1H-indazol-1- yl)phenyl)-3-methylazetidine-3-carbonitrile |
| 23.28 | 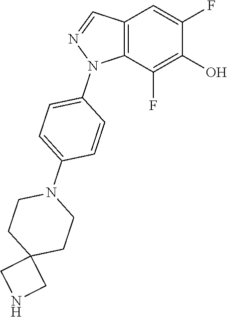 [SMILES: Oc1c(F)cc2cnn(-c3ccc(N4CCC5(CC4)CNC5)cc3)c2c1F] | 1-(4-(2,7-Diazaspiro[3.5]nonan-7- yl)phenyl)-5,7-difluoro-1H-indazol-6-ol |
| 24 | 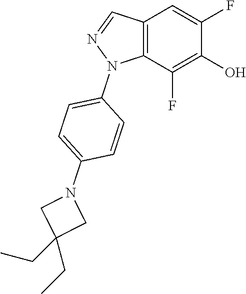 [SMILES: CCC1(CC)CN(c2ccc(-n3ncc4cc(F)c(O)c(F)c43)cc2)C1] | 1-(4-(3,3-Diethylazetidin-1-yl)phenyl)-5,7- difluoro-1H-indazol-6-ol |
| 24.1 | 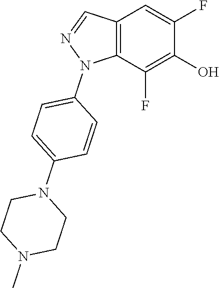 [SMILES: CN1CCN(c2ccc(-n3ncc4cc(F)c(O)c(F)c43)cc2)CC1] | 5,7-Difluoro-1-(4-(4-methylpiperazin-1- yl)phenyl)-1H-indazol-6-ol |
| 24.2 | 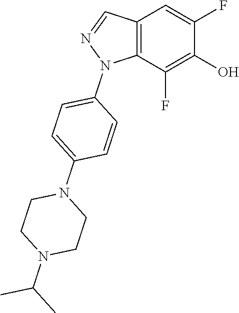 [SMILES: CC(C)N1CCN(c2ccc(-n3ncc4cc(F)c(O)c(F)c43)cc2)CC1] | 5,7-Difluoro-1-(4-(4-isopropylpiperazin-1- yl)phenyl)-1H-indazol-6-ol |
| 24.3 | 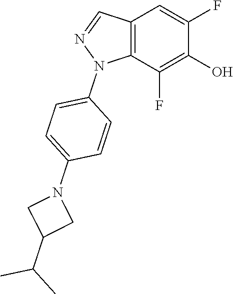 [SMILES: CC(C)C1CN(c2ccc(-n3ncc4cc(F)c(O)c(F)c43)cc2)C1] | 5,7-Difluoro-1-(4-(3-isopropylazetidin-1- yl)phenyl)-1H-indazol-6-ol |
| 24.4 | 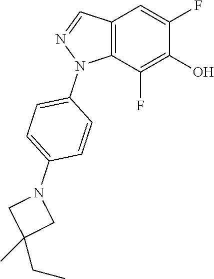 [SMILES: CCC1(C)CN(c2ccc(-n3ncc4cc(F)c(O)c(F)c43)cc2)C1] | 1-(4-(3-Ethyl-3-methylazetidin-1-yl)phenyl)- 5,7-difluoro-1H-indazol-6-ol |
| 24.5 | 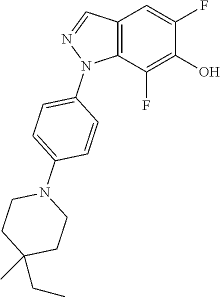 [SMILES: CCC1(C)CCN(c2ccc(-n3ncc4cc(F)c(O)c(F)c43)cc2)CC1] | 1-(4-(4-Ethyl-4-methylpiperidin-1- yl)phenyl)-5,7-difluoro-1H-indazol-6-ol |
| 24.6 | 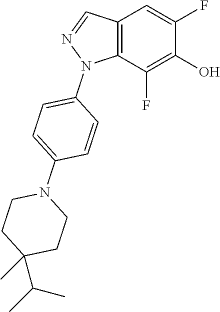 [SMILES: CC(C)C1(C)CCN(c2ccc(-n3ncc4cc(F)c(O)c(F)c43)cc2)CC1] | 5,7-Difluoro-1-(4-(4-isopropyl-4- methylpiperidin-1-yl)phenyl)-1H-indazol-6- ol |
| 24.7 | 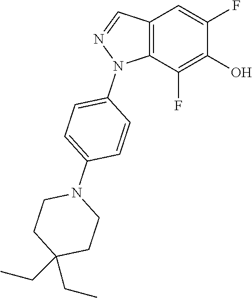 [SMILES: CCC1(CC)CCN(c2ccc(-n3ncc4cc(F)c(O)c(F)c43)cc2)CC1] | 1-(4-(4,4-Diethylpiperidin-1-yl)phenyl)-5,7- difluoro-1H-indazol-6-ol |
| 24.8 | 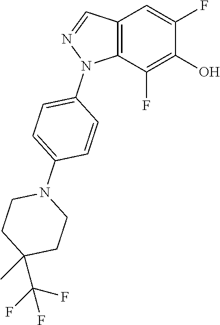 [SMILES: CC1(C(F)(F)F)CCN(c2ccc(-n3ncc4cc(F)c(O)c(F)c43)cc2)CC1] | 5,7-Difluoro-1-(4-(4-methyl-4- (trifluoromethyl)piperidin-1-yl)phenyl)-1H- indazol-6-ol |
| 24.9 | 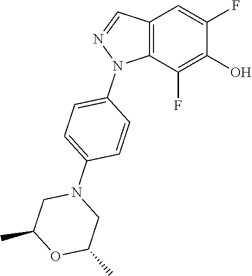 [SMILES: C[C@H]1CN(c2ccc(-n3ncc4cc(F)c(O)c(F)c43)cc2)C[C@H](C)O1] | 1-(4-((2S,6S)-2,6- Dimethylmorpholino)phenyl)-5,7-difluoro- 1H-indazol-6-ol |
| 24.10 | 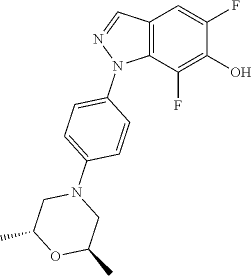 [SMILES: C[C@@H]1CN(c2ccc(-n3ncc4cc(F)c(O)c(F)c43)cc2)C[C@@H](C)O1] | 1-(4-((2R,6R)-2,6- Dimethylmorpholino)phenyl)-5,7-difluoro- 1H-indazol-6-ol |
| 24.11 | 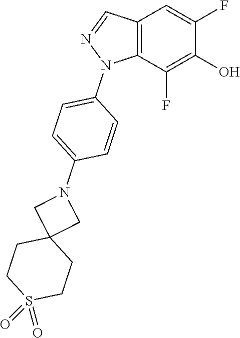 [SMILES: O=S1(=O)CCC2(CC1)CN(c1ccc(-n3ncc4cc(F)c(O)c(F)c43)cc1)C2] | 2-(4-(5,7-Difluoro-6-hydroxy-1H-indazol-1- yl)phenyl)-7-thia-2-azaspiro[3.5]nonane 7,7- dioxide |
| 24.12 | 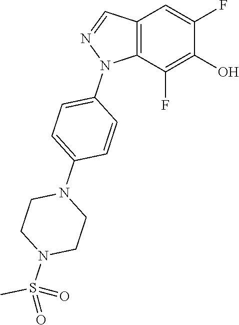 [SMILES: CS(=O)(=O)N1CCN(c2ccc(-n3ncc4cc(F)c(O)c(F)c43)cc2)CC1] | 5-Fluoro-7-methyl-1-(4-(4- (methylsulfonyl)piperazin-1-yl)phenyl)-1H- indazol-6-ol |
| 25 | 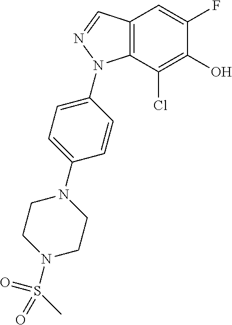 [SMILES: CS(=O)(=O)N1CCN(c2ccc(-n3ncc4cc(F)c(O)c(Cl)c43)cc2)CC1] | 7-Chloro-5-fluoro-1-(4-(4- (methylsulfonyl)piperazin-1-yl)phenyl)-1H- indazol-6-ol |
| 25.1 | 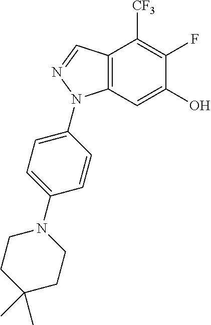 [SMILES: CC1(C)CCN(c2ccc(-n3ncc4c(C(F)(F)F)c(F)c(O)cc43)cc2)CC1] | 1-(4-(4,4-Dimethylpiperidin-1-yl)phenyl)-5- fluoro-4-(trifluoromethyl)-1H-indazol-6-ol |
| 25.2 | 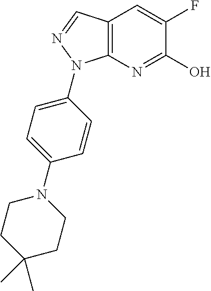 [SMILES: CC1(C)CCN(c2ccc(-n3ncc4cc(F)c(O)nc43)cc2)CC1] | 1-(4-(4,4-Dimethylpiperidin-1-yl)phenyl)-5- fluoro-1H-pyrazolo[3,4-b]pyridin-6-ol |
| 25.3 | 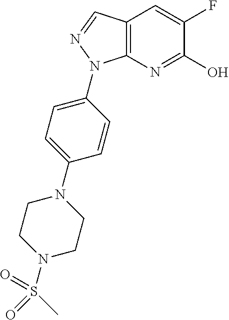 [SMILES: CS(=O)(=O)N1CCN(c2ccc(-n3ncc4cc(F)c(O)nc43)cc2)CC1] | 5-Fluoro-1-(4-(4-(methylsulfonyl)piperazin- 1-yl)phenyl)-1H-pyrazolo[3,4-b]pyridin-6-ol |
| 25.4 | 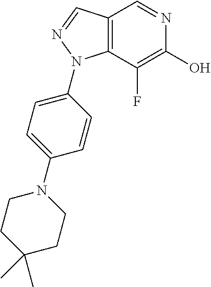 [SMILES: CC1(C)CCN(c2ccc(-n3ncc4cnc(O)c(F)c43)cc2)CC1] | 1-(4-(4,4-Dimethylpiperidin-1-yl)phenyl)-7- fluoro-1H-pyrazolo[4,3-c]pyridin-6-ol |
| 25.5 | 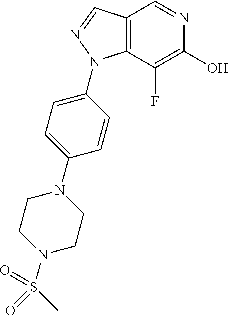 [SMILES: CS(=O)(=O)N1CCN(c2ccc(-n3ncc4cnc(O)c(F)c43)cc2)CC1] | 7-Fluoro-1-(4-(4-(methylsulfonyl)piperazin- 1-yl)phenyl)-1H-pyrazolo[4,3-c]pyridin-6-ol |
| 25.6 | 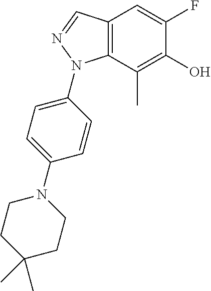 [SMILES: Cc1c(O)c(F)cc2cnn(-c3ccc(N4CCC(C)(C)CC4)cc3)c12] | 1-(4-(4,4-Dimethylpiperidin-1-yl)phenyl)-5- fluoro-7-methyl-1H-indazol-6-ol |
| 26 | 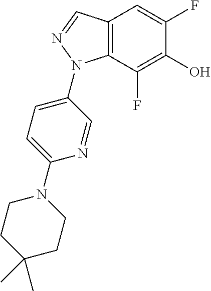 [SMILES: CC1(C)CCN(c2ccc(-n3ncc4cc(F)c(O)c(F)c43)cn2)CC1] | 1-(6-(4,4-Dimethylpiperidin-1-yl)pyridin-3- yl)-5,7-difluoro-1H-indazol-6-ol |
| 26.1 | 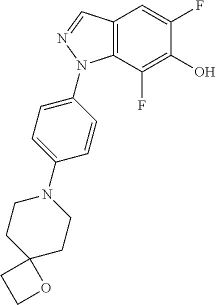 [SMILES: Oc1c(F)cc2cnn(-c3ccc(N4CCC5(CCO5)CC4)cc3)c2c1F] | 1-(4-(1-Oxa-7-azaspiro[3.5]nonan-7- yl)phenyl)-5,7-difluoro-1H-indazol-6-ol |
| 26.2 | 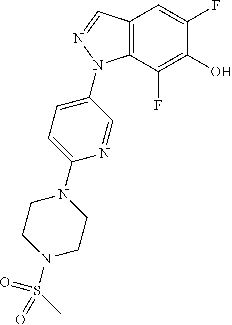 [SMILES: CS(=O)(=O)N1CCN(c2ccc(-n3ncc4cc(F)c(O)c(F)c43)cn2)CC1] | 5,7-Difluoro-1-(6-(4- (methylsulfonyl)piperazin-1-yl)pyridin-3- yl)-1H-indazol-6-ol |
| 26.3 | 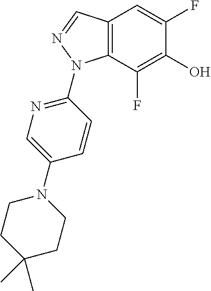 [SMILES: CC1(C)CCN(c2ccc(-n3ncc4cc(F)c(O)c(F)c43)nc2)CC1] | 1-(5-(4,4-Dimethylpiperidin-1-yl)pyridin-2- yl)-5,7-difluoro-1H-indazol-6-ol |
| 26.4 | 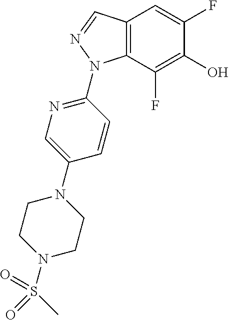 [SMILES: CS(=O)(=O)N1CCN(c2ccc(-n3ncc4cc(F)c(O)c(F)c43)nc2)CC1] | 5,7-Difluoro-1-(5-(4- (methylsulfonyl)piperazin-1-yl)pyridin-2- yl)-1H-indazol-6-ol |
| 26.5 | 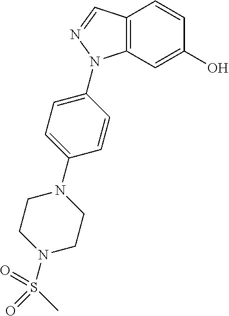 [SMILES: CS(=O)(=O)N1CCN(c2ccc(-n3ncc4ccc(O)cc43)cc2)CC1] | 1-(4-(4-(Methylsulfonyl)piperazin-1- yl)phenyl)-1H-indazol-6-ol |
| 26.6 | 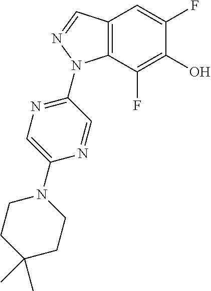 [SMILES: CC1(C)CCN(c2cnc(-n3ncc4cc(F)c(O)c(F)c43)cn2)CC1] | 1-(5-(4,4-Dimethylpiperidin-1-yl)pyrazin-2- yl)-5,7-difluoro-1H-indazol-6-ol |
| 26.7 | 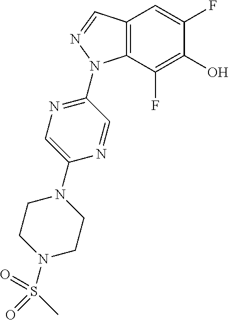 [SMILES: CS(=O)(=O)N1CCN(c2cnc(-n3ncc4cc(F)c(O)c(F)c43)cn2)CC1] | 5,7-Difluoro-1-(5-(4- (methylsulfonyl)piperazin-1-yl)pyrazin-2- yl)-1H-indazol-6-ol |
| 26.8 | 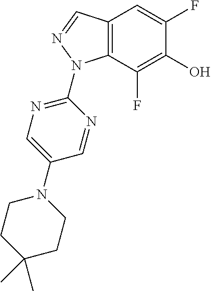 [SMILES: CC1(C)CCN(c2cnc(-n3ncc4cc(F)c(O)c(F)c43)nc2)CC1] | 1-(5-(4,4-Dimethylpiperidin-1-yl)pyrimidin- 2-yl)-5,7-difluoro-1H-indazol-6-ol |
| 26.9 | 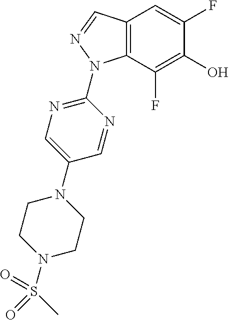 [SMILES: CS(=O)(=O)N1CCN(c2cnc(-n3ncc4cc(F)c(O)c(F)c43)nc2)CC1] | 5,7-Difluoro-1-(5-(4- (methylsulfonyl)piperazin-1-yl)pyrimidin-2- yl)-1H-indazol-6-ol |
| 26.10 | 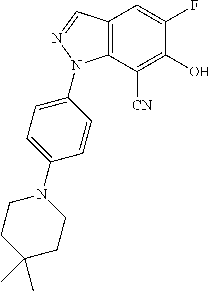 [SMILES: CC1(C)CCN(c2ccc(-n3ncc4cc(F)c(O)c(C#N)c43)cc2)CC1] | 1-(4-(4,4-Dimethylpiperidin-1-yl)phenyl)-5- fluoro-6-hydroxy-1H-indazole-7-carbonitrile |
| 26.11 | 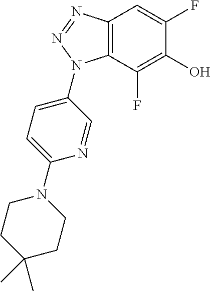 [SMILES: CC1(C)CCN(c2ccc(-n3nnc4cc(F)c(O)c(F)c43)cn2)CC1] | 1-(6-(4,4-Dimethylpiperidin-1-yl)pyridin-3- yl)-5,7-difluoro-1H-benzo[d][1,2,3]triazol-6- ol |
| 26.12 | 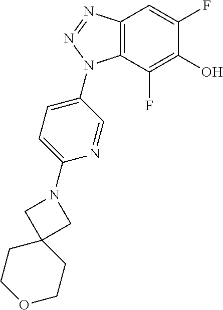 [SMILES: Oc1c(F)cc2nnn(-c3ccc(N4CC5(CCOCC5)C4)nc3)c2c1F] | 1-(6-(7-Oxa-2-azaspiro[3.5]nonan-2- yl)pyridin-3-yl)-5,7-difluoro-1H- benzo[d][1,2,3]triazol-6-ol |
| 26.13 | 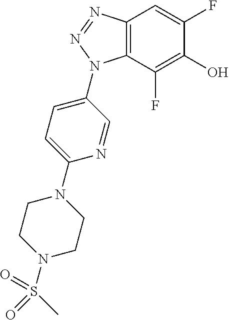 [SMILES: CS(=O)(=O)N1CCN(c2ccc(-n3nnc4cc(F)c(O)c(F)c43)cn2)CC1] | 5,7-Difluoro-1-(6-(4- (methylsulfonyl)piperazin-1-yl)pyridin-3- yl)-1H-benzo[d][1,2,3]triazol-6-ol |
| 26.14 | 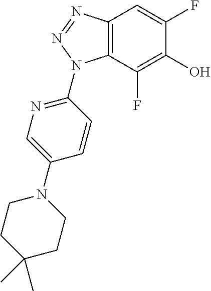 [SMILES: CC1(C)CCN(c2ccc(-n3nnc4cc(F)c(O)c(F)c43)nc2)CC1] | 1-(5-(4,4-Dimethylpiperidin-1-yl)pyridin-2- yl)-5,7-difluoro-1H-benzo[d][1,2,3]triazol-6- ol |
| 26.15 | 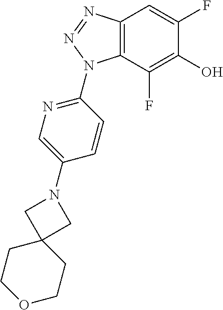 [SMILES: Oc1c(F)cc2nnn(-c3ccc(N4CC5(CCOCC5)C4)cn3)c2c1F] | 1-(5-(7-Oxa-2-azaspiro[3.5]nonan-2- yl)pyridin-2-yl)-5,7-difluoro-1H- benzo[d][1,2,3]triazol-6-ol |
| 26.16 | 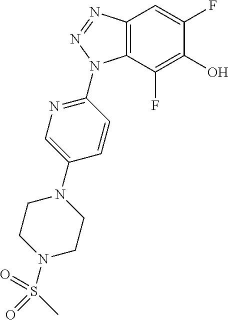 [SMILES: CS(=O)(=O)N1CCN(c2ccc(-n3nnc4cc(F)c(O)c(F)c43)nc2)CC1] | 5,7-Difluoro-1-(5-(4- (methylsulfonyl)piperazin-1-yl)pyridin-2- yl)-1H-benzo[d][1,2,3]triazol-6-ol |
| 26.17 | 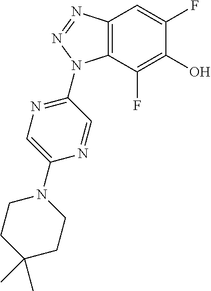 [SMILES: CC1(C)CCN(c2cnc(-n3nnc4cc(F)c(O)c(F)c43)cn2)CC1] | 1-(5-(4,4-Dimethylpiperidin-1-yl)pyrazin-2- yl)-5,7-difluoro-1H-benzo[d][1,2,3]triazol-6- ol |
| 26.18 | 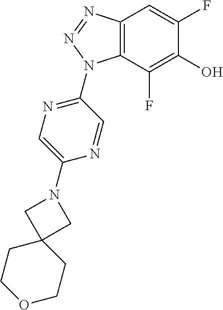 [SMILES: Oc1c(F)cc2nnn(-c3cnc(N4CC5(CCOCC5)C4)cn3)c2c1F] | 1-(5-(7-Oxa-2-azaspiro[3.5]nonan-2- yl)pyrazin-2-yl)-5,7-difluoro-1H- benzo[d][1,2,3]triazol-6-ol |
| 26.19 | 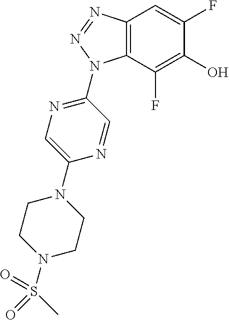 [SMILES: CS(=O)(=O)N1CCN(c2cnc(-n3nnc4cc(F)c(O)c(F)c43)cn2)CC1] | 5,7-Difluoro-1-(5-(4- (methylsulfonyl)piperazin-1-yl)pyrazin-2- yl)-1H-benzo[d][1,2,3]triazol-6-ol |
| 26.20 | 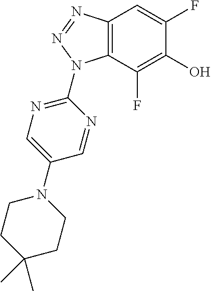 [SMILES: CC1(C)CCN(c2cnc(-n3nnc4cc(F)c(O)c(F)c43)nc2)CC1] | 1-(5-(4,4-Dimethylpiperidin-1-yl)pyrimidin- 2-yl)-5,7-difluoro-1H-benzo[d][1,2,3]triazol- 6-ol |
| 26.21 | 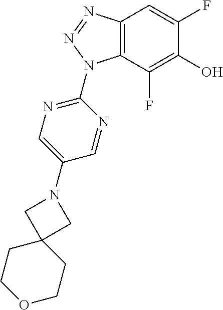 [SMILES: Oc1c(F)cc2nnn(-c3ncc(N4CC5(CCOCC5)C4)cn3)c2c1F] | 1-(5-(7-Oxa-2-azaspiro[3.5]nonan-2- yl)pyrimidin-2-yl)-5,7-difluoro-1H- benzo[d][1,2,3]triazol-6-ol |
| 26.22 | 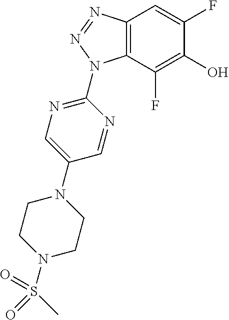 [SMILES: CS(=O)(=O)N1CCN(c2cnc(-n3nnc4cc(F)c(O)c(F)c43)nc2)CC1] | 5,7-Difluoro-1-(5-(4- (methylsulfonyl)piperazin-1-yl)pyrimidin-2- yl)-1H-benzo[d][1,2,3]triazol-6-ol |
| 26.23 | 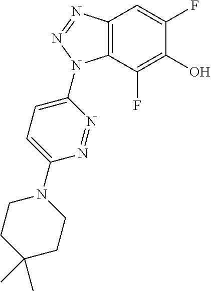 [SMILES: CC1(C)CCN(c2ccc(-n3nnc4cc(F)c(O)c(F)c43)nn2)CC1] | 1-(6-(4,4-Dimethylpiperidin-1-yl)pyridazin- 3-yl)-5,7-difluoro-1H-benzo[d][1,2,3]triazol- 6-ol |
| 26.24 | 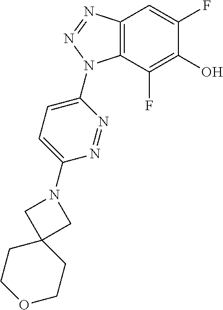 [SMILES: Oc1c(F)cc2nnn(-c3ccc(N4CC5(CCOCC5)C4)nn3)c2c1F] | 1-(6-(7-Oxa-2-azaspiro[3.5]nonan-2- yl)pyridazin-3-yl)-5,7-difluoro-1H- benzo[d][1,2,3]triazol-6-ol |
| 26.25 | 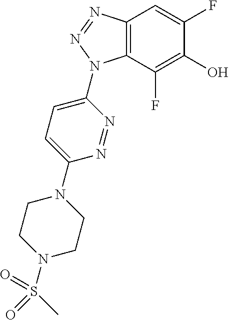 [SMILES: CS(=O)(=O)N1CCN(c2ccc(-n3nnc4cc(F)c(O)c(F)c43)nn2)CC1] | 5,7-Difluoro-1-(6-(4- (methylsulfonyl)piperazin-1-yl)pyridazin-3- yl)-1H-benzo[d][1,2,3]triazol-6-ol |
| 26.26 | 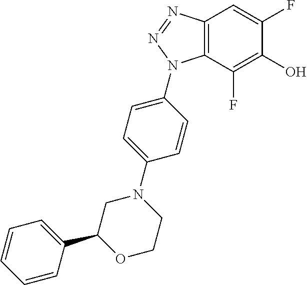 [SMILES: Oc1c(F)cc2nnn(-c3ccc(N4CCO[C@@H](c5ccccc5)C4)cc3)c2c1F] | (S)-5,7-Difluoro-1-(4-(2- phenylmorpholino)phenyl)-1H- benzo[d][1,2,3]triazol-6-ol |
| 26.27 | 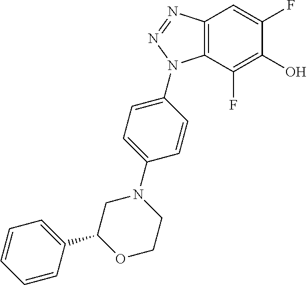 [SMILES: Oc1c(F)cc2nnn(-c3ccc(N4CCO[C@H](c5ccccc5)C4)cc3)c2c1F] | (R)-5,7-Difluoro-1-(4-(2- phenylmorpholino)phenyl)-1H- benzo[d][1,2,3]triazol-6-ol |
| 26.28 | 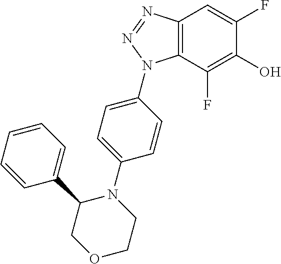 [SMILES: Oc1c(F)cc2nnn(-c3ccc(N4CCOC[C@H]4c4ccccc4)cc3)c2c1F] | (R)-5,7-Difluoro-1-(4-(3- phenylmorpholino)phenyl)-1H- benzo[d][1,2,3]triazol-6-ol |
| 26.29 | 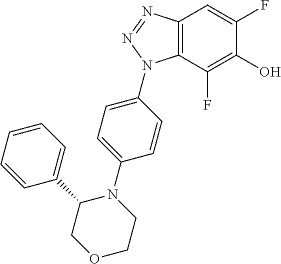 [SMILES: Oc1c(F)cc2nnn(-c3ccc(N4CCOC[C@@H]4c4ccccc4)cc3)c2c1F] | (S)-5,7-Difluoro-1-(4-(3- phenylmorpholino)phenyl)-1H- benzo[d][1,2,3]triazol-6-ol |
| 27 | 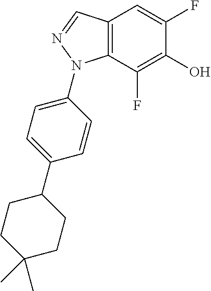 [SMILES: CC1(C)CCC(c2ccc(-n3ncc4cc(F)c(O)c(F)c43)cc2)CC1] | 1-(4-(4,4-Dimethylcyclohexyl)phenyl)-5,7- difluoro-1H-indazol-6-ol |
| 27.1 | 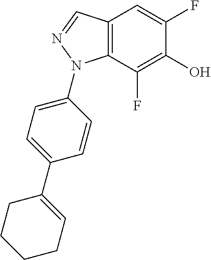 [SMILES: Oc1c(F)cc2cnn(-c3ccc(C4=CCCCC4)cc3)c2c1F] | 5,7-Difluoro-1-(2′,3′,4′,5′-tetrahydro-[1,1′- biphenyl]-4-yl)-1H-indazol-6-ol |
| 27.2 | 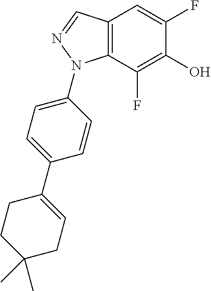 [SMILES: CC1(C)CC=C(c2ccc(-n3ncc4cc(F)c(O)c(F)c43)cc2)CC1] | 1-(4′,4′-Dimethyl-2′,3′,4′,5′-tetrahydro-[1,1′- biphenyl]-4-yl)-5,7-difluoro-1H-indazol-6-ol |
| 27.3 | 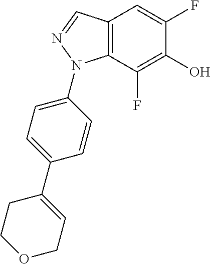 [SMILES: Oc1c(F)cc2cnn(-c3ccc(C4=CCOCC4)cc3)c2c1F] | 1-(4-(3,6-Dihydro-2H-pyran-4-yl)phenyl)- 5,7-difluoro-1H-indazol-6-ol |
| 27.4 | 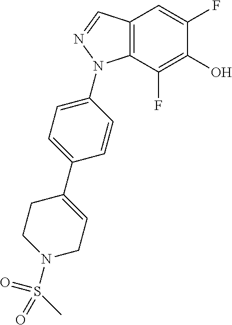 [SMILES: CS(=O)(=O)N1CC=C(c2ccc(-n3ncc4cc(F)c(O)c(F)c43)cc2)CC1] | 5,7-Difluoro-1-(4-(1-(methylsulfonyl)- 1,2,3,6-tetrahydropyridin-4-yl)phenyl)-1H- indazol-6-ol |
| 27.5 | 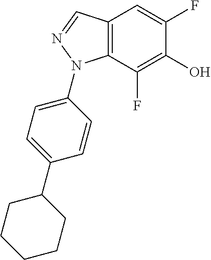 [SMILES: Oc1c(F)cc2cnn(-c3ccc(C4CCCCC4)cc3)c2c1F] | 1-(4-Cyclohexylphenyl)-5,7-difluoro-1H- indazol-6-ol |
| 27.6 | 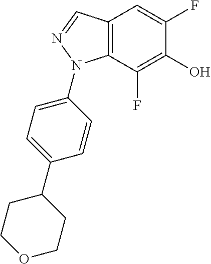 [SMILES: Oc1c(F)cc2cnn(-c3ccc(C4CCOCC4)cc3)c2c1F] | 5,7-Difluoro-1-(4-(tetrahydro-2H-pyran-4- yl)phenyl)-1H-indazol-6-ol |
| 27.7 | 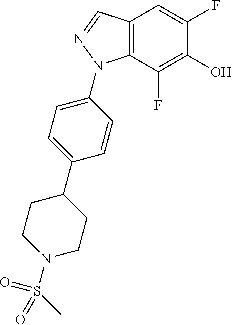 [SMILES: CS(=O)(=O)N1CCC(c2ccc(-n3ncc4cc(F)c(O)c(F)c43)cc2)CC1] | 5,7-Difluoro-1-(4-(1- (methylsulfonyl)piperidin-4-yl)phenyl)-1H- indazol-6-ol |
| 27.8 | 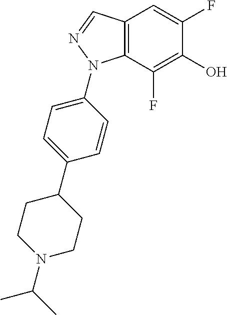 [SMILES: CC(C)N1CCC(c2ccc(-n3ncc4cc(F)c(O)c(F)c43)cc2)CC1] | 5,7-Difluoro-1-(4-(1-isopropylpiperidin-4- yl)phenyl)-1H-indazol-6-ol |
| 27.9 | 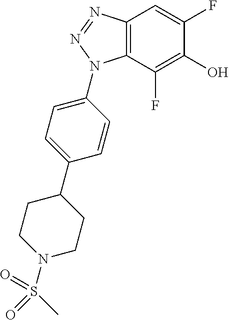 [SMILES: CS(=O)(=O)N1CCC(c2ccc(-n3nnc4cc(F)c(O)c(F)c43)cc2)CC1] | 5,7-Difluoro-1-(4-(1- (methylsulfonyl)piperidin-4-yl)phenyl)-1H- benzo[d][1,2,3]triazol-6-ol |
| 27.10 | 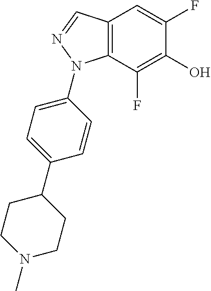 [SMILES: CN1CCC(c2ccc(-n3ncc4cc(F)c(O)c(F)c43)cc2)CC1] | 5,7-Difluoro-1-(4-(1-methylpiperidin-4- yl)phenyl)-1H-indazol-6-ol |
| 28 | 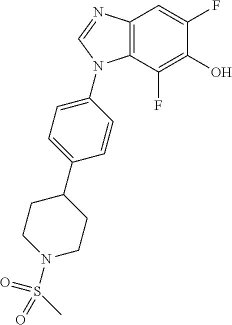 [SMILES: CS(=O)(=O)N1CCC(c2ccc(-n3cnc4cc(F)c(O)c(F)c43)cc2)CC1] | 5,7-Difluoro-1-(4-(1- (methylsulfonyl)piperidin-4-yl)phenyl)-1H- benzo[d]imidazol-6-ol |
| 29 | 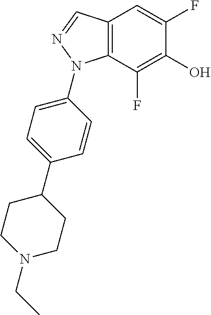 [SMILES: CCN1CCC(c2ccc(-n3ncc4cc(F)c(O)c(F)c43)cc2)CC1] | 1-(4-(1-Ethylpiperidin-4-yl)phenyl)-5,7- difluoro-1H-indazol-6-ol |
| 30 | 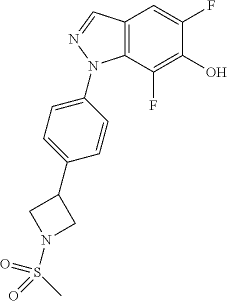 [SMILES: CS(=O)(=O)N1CC(c2ccc(-n3ncc4cc(F)c(O)c(F)c43)cc2)C1] | 5,7-Difluoro-1-(4-(1- (methylsulfonyl)azetidin-3-yl)phenyl)-1H- indazol-6-ol |
| 30.1 | 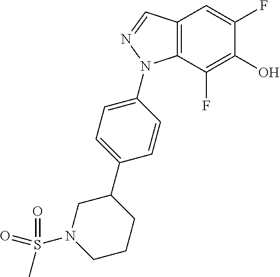 [SMILES: CS(=O)(=O)N1CCCC(c2ccc(-n3ncc4cc(F)c(O)c(F)c43)cc2)C1] | 5,7-Difluoro-1-(4-(1- (methylsulfonyl)piperidin-3-yl)phenyl)-1H- indazol-6-ol |
| 31 | 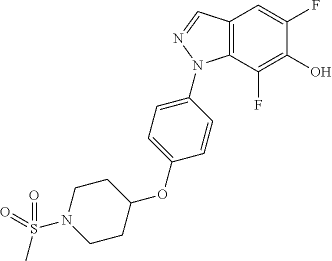 [SMILES: CS(=O)(=O)N1CCC(Oc2ccc(-n3ncc4cc(F)c(O)c(F)c43)cc2)CC1] | 5,7-Difluoro-1-(4-((1- (methylsulfonyl)piperidin-4-yl)oxy)phenyl)- 1H-indazol-6-ol |
| 32 | 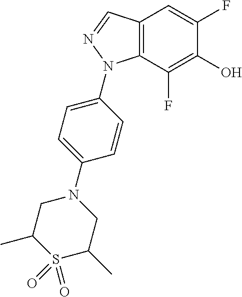 [SMILES: CC1CN(c2ccc(-n3ncc4cc(F)c(O)c(F)c43)cc2)CC(C)S1(=O)=O] | 4-(4-(5,7-Difluoro-6-hydroxy-1H-indazol-1- yl)phenyl)-2,6-dimethylthiomorpholine 1,1- dioxide |
| 32.1 | 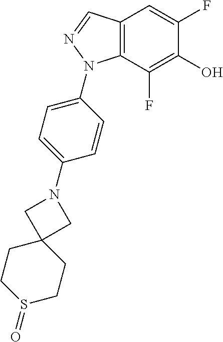 [SMILES: O=S1CCC2(CC1)CN(c1ccc(-n3ncc4cc(F)c(O)c(F)c43)cc1)C2] | 2-(4-(5,7-Difluoro-6-hydroxy-1H-indazol-1- yl)phenyl)-7-thia-2-azaspiro[3.5]nonane 7- oxide |
| 32.2 | 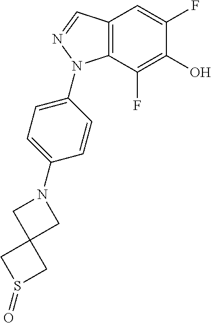 [SMILES: O=S1CC2(CN(c3ccc(-n4ncc5cc(F)c(O)c(F)c54)cc3)C2)C1] | 6-(4-(5,7-Difluoro-6-hydroxy-1H-indazol-1- yl)phenyl)-2-thia-6-azaspiro[3.3]heptane 2- oxide |
| 32.3 | 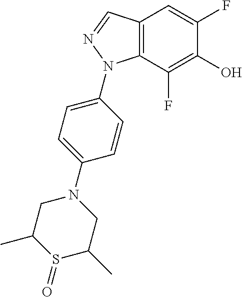 [SMILES: CC1CN(c2ccc(-n3ncc4cc(F)c(O)c(F)c43)cc2)CC(C)S1=O] | 4-(4-(5,7-Difluoro-6-hydroxy-1H-indazol-1- yl)phenyl)-2,6-dimethylthiomorpholine 1- oxide |
| 32.4 | 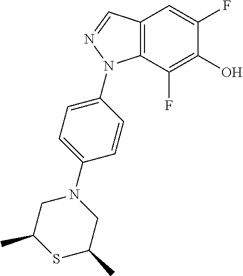 [SMILES: C[C@@H]1CN(c2ccc(-n3ncc4cc(F)c(O)c(F)c43)cc2)C[C@H](C)S1] | 1-(4-(cis-2,6- Dimethylthiomorpholino)phenyl)-5,7- difluoro-1H-indazol-6-ol |
| 32.5 | 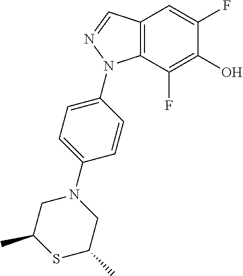 [SMILES: C[C@H]1CN(c2ccc(-n3ncc4cc(F)c(O)c(F)c43)cc2)C[C@H](C)S1] | 1-(4-(trans-2,6- Dimethylthiomorpholino)phenyl)-5,7- difluoro-1H-indazol-6-ol |
| 33 | 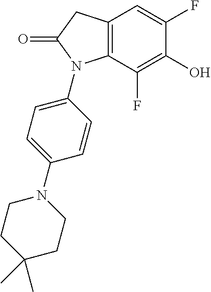 [SMILES: CC1(C)CCN(c2ccc(N3C(=O)Cc4cc(F)c(O)c(F)c43)cc2)CC1] | 1-(4-(4,4-Dimethylpiperidin-1-yl)phenyl)- 5,7-difluoro-6-hydroxyindolin-2-one |
| 34 | 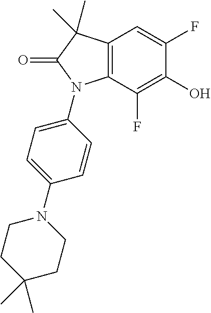 [SMILES: CC1(C)CCN(c2ccc(N3C(=O)C(C)(C)c4cc(F)c(O)c(F)c43)cc2)CC1] | 1-(4-(4,4-Dimethylpiperidin-1-yl)phenyl)- 5,7-difluoro-6-hydroxy-3,3-dimethylindolin- 2-one |
| 35 | 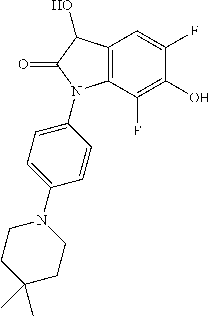 [SMILES: CC1(C)CCN(c2ccc(N3C(=O)C(O)c4cc(F)c(O)c(F)c43)cc2)CC1] | 1-(4-(4,4-Dimethylpiperidin-1-yl)phenyl)- 5,7-difluoro-3,6-dihydroxyindolin-2-one hydrochloride |
| 36 | 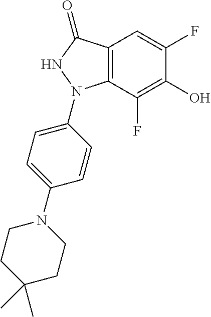 [SMILES: CC1(C)CCN(c2ccc(-n3[nH]c(=O)c4cc(F)c(O)c(F)c43)cc2)CC1] | 1-(4-(4,4-Dimethylpiperidin-1-yl)phenyl)- 5,7-difluoro-6-hydroxy-1H-indazol-3(2H)- one |
| 37 | 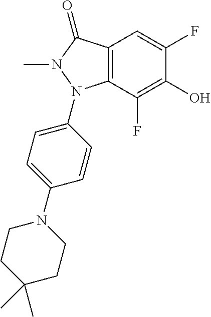 [SMILES: Cn1c(=O)c2cc(F)c(O)c(F)c2n1-c1ccc(N2CCC(C)(C)CC2)cc1] | 1-(4-(4,4-Dimethylpiperidin-1-yl)phenyl)- 5,7-difluoro-6-hydroxy-2-methyl-1H- indazol-3(2H)-one |
| 37.1 | 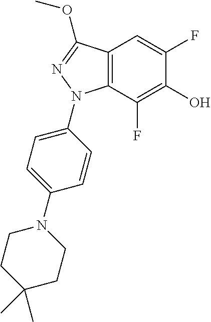 [SMILES: COc1nn(-c2ccc(N3CCC(C)(C)CC3)cc2)c2c(F)c(O)c(F)cc12] | 1-(4-(4,4-Dimethylpiperidin-1-yl)phenyl)- 5,7-difluoro-3-methoxy-1H-indazol-6-ol |
| 38 | 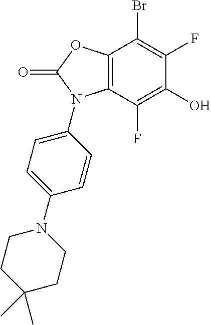 [SMILES: CC1(C)CCN(c2ccc(-n3c(=O)oc4c(Br)c(F)c(O)c(F)c43)cc2)CC1] | 7-Bromo-3-(4-(4,4-dimethylpiperidin-1- yl)phenyl)-4,6-difluoro-5- hydroxybenzo[d]oxazol-2(3H)-one |
| 39 | 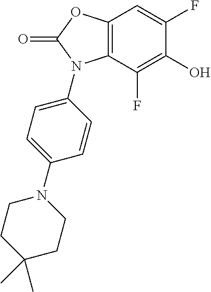 [SMILES: CC1(C)CCN(c2ccc(-n3c(=O)oc4cc(F)c(O)c(F)c43)cc2)CC1] | 3-(4-(4,4-Dimethylpiperidin-1-yl)phenyl)- 4,6-difluoro-5-hydroxybenzo[d]oxazol- 2(3H)-one |
| 40 | 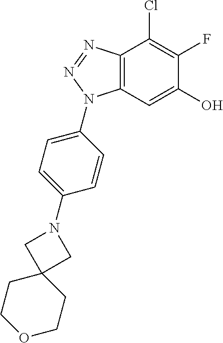 [SMILES: Oc1cc2c(nnn2-c2ccc(N3CC4(CCOCC4)C3)cc2)c(Cl)c1F] | 1-(4-(7-Oxa-2-azaspiro[3.5]nonan-2- yl)phenyl)-4-chloro-5-fluoro-1H- benzo[d][1,2,3]triazol-6-ol |
| 40.1 | 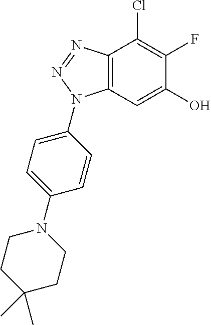 [SMILES: CC1(C)CCN(c2ccc(-n3nnc4c(Cl)c(F)c(O)cc43)cc2)CC1] | 4-Chloro-1-(4-(4,4-dimethylpiperidin-1- yl)phenyl)-5-fluoro-1H- benzo[d][1,2,3]triazol-6-ol |
| 40.2 | 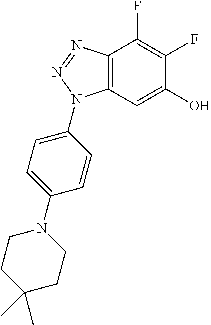 [SMILES: CC1(C)CCN(c2ccc(-n3nnc4c(F)c(F)c(O)cc43)cc2)CC1] | 1-(4-(4,4-Dimethylpiperidin-1-yl)phenyl)- 4,5-difluoro-1H-benzo[d][1,2,3]triazol-6-ol |
| 40.3 | 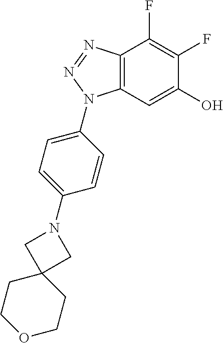 [SMILES: Oc1cc2c(nnn2-c2ccc(N3CC4(CCOCC4)C3)cc2)c(F)c1F] | 1-(4-(7-oxa-2-azaspiro[3.5]nonan-2- yl)phenyl)-4,5-difluoro-1H- benzo[d][1,2,3]triazol-6-ol |
| 41 | 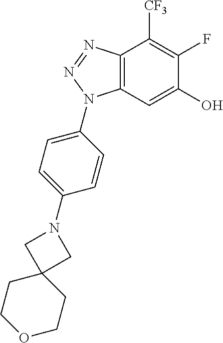 [SMILES: Oc1cc2c(nnn2-c2ccc(N3CC4(CCOCC4)C3)cc2)c(C(F)(F)F)c1F] | 1-(4-(7-Oxa-2-azaspiro[3.5]nonan-2- yl)phenyl)-5-fluoro-4-(trifluoromethyl)-1H- benzo[d][1,2,3]triazol-6-ol |
| 41.1 | 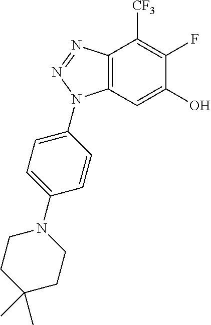 [SMILES: CC1(C)CCN(c2ccc(-n3nnc4c(C(F)(F)F)c(F)c(O)cc43)cc2)CC1] | 1-(4-(4,4-Dimethylpiperidin-1-yl)phenyl)-5- fluoro-4-(trifluoromethyl)-1H- benzo[d][1,2,3]triazol-6-ol |
| 42 | 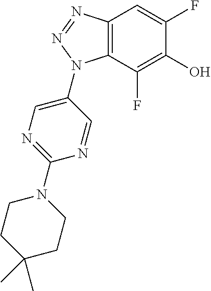 [SMILES: CC1(C)CCN(c2ncc(-n3nnc4cc(F)c(O)c(F)c43)cn2)CC1] | 1-(2-(4,4-Dimethylpiperidin-1-yl)pyrimidin- 5-yl)-5,7-difluoro-1H-benzo[d][1,2,3]triazol- 6-ol |
| 42.1 | 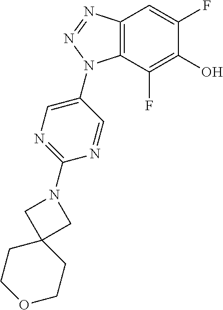 [SMILES: Oc1c(F)cc2nnn(-c3cnc(N4CC5(CCOCC5)C4)nc3)c2c1F] | 1-(2-(7-Oxa-2-azaspiro[3.5]nonan-2- yl)pyrimidin-5-yl)-5,7-difluoro-1H- benzo[d][1,2,3]triazol-6-ol |

In some embodiments, provided herein is a pharmaceutically acceptable salt or solvate of a compound that is described in Table 1.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal fluids than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxy ethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with abase to provide a “pharmaceutically acceptable base addition salt.”

In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic groups (e.g., alkyl groups, aromatic rings) of compounds described herein are susceptible to various metabolic reactions. Incorporation of appropriate substituents on the organic groups will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. The prodrug may be a substrate for a transporter. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound. In some embodiments, a prodrug of the compound disclosed herein permits targeted delivery of the compound to a particular region of the gastrointestinal tract. Formation of a pharmacologically active metabolite by the colonic metabolism of drugs is a commonly used “prodrug” approach for the colon-specific drug delivery systems.

In some embodiments, a prodrug is formed by the formation of a covalent linkage between drug and a carrier in such a manner that upon oral administration the moiety remains intact in the stomach and small intestine. This approach involves the formation of a prodrug, which is a pharmacologically inactive derivative of a parent drug molecule that requires spontaneous or enzymatic transformation in the biological environment to release the active drug. Formation of prodrugs has improved delivery properties over the parent drug molecule. The problem of stability of certain drugs from the adverse environment of the upper gastrointestinal tract can be eliminated by prodrug formation, which is converted into the parent drug molecule once it reaches the colon. Site specific drug delivery through site specific prodrug activation may be accomplished by the utilization of some specific property at the target site, such as altered pH or high activity of certain enzymes relative to the non-target tissues for the prodrug-drug conversion.

In some embodiments, covalent linkage of the drug with a carrier forms a conjugate. Such conjugates include, but are not limited to, azo bond conjugates, glycoside conjugates, glucuronide conjugates, cyclodextrin conjugates, dextran conjugates or amino-acid conjugates.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

In additional or further embodiments, the compounds are rapidly metabolized in plasma.

In additional or further embodiments, the compounds are rapidly metabolized by the intestines.

In additional or further embodiments, the compounds are rapidly metabolized by the liver.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

The compounds described herein are prepared by the general synthetic routes described below in Schemes 1 to 13.

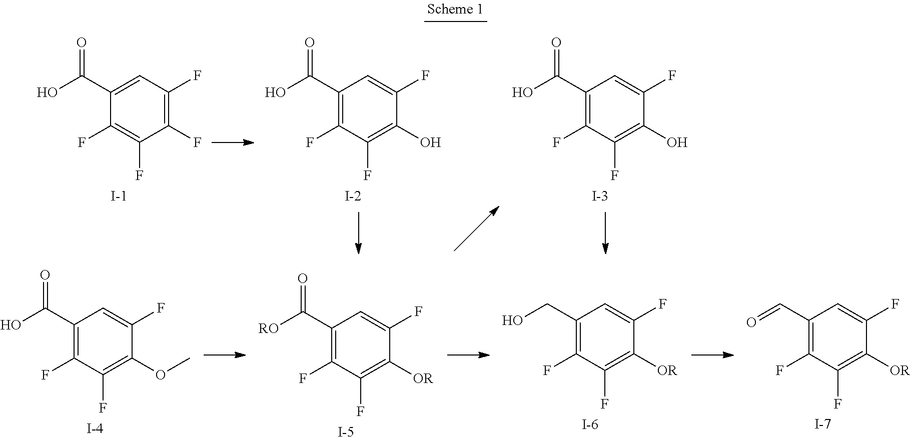

In some embodiments, compounds described herein are prepared as outlined in Scheme 1.

In some embodiments, intermediate I-1 is reacted under appropriate nucleophilic aromatic substitution reaction conditions to provide intermediate I-2, followed by installation of an appropriate protecting group to provide intermediate I-5. In some embodiments, appropriate nucleophilic aromatic substitution reaction conditions include using an appropriate base in an appropriate solvent at an appropriate time and at an appropriate temperature. In some embodiments, the appropriate base is sodium hydroxide. In some embodiments, the appropriate solvent is water. In some embodiments, the appropriate temperature is 100° C. and the appropriate amount of time stirred is about 15 hours (overnight).

In some embodiments, the protecting group is a MOM protecting group. In some embodiments, appropriate conditions to install a MOM protecting group include using an appropriate reagent and an appropriate base in an appropriate solvent at an appropriate temperature and amount of time. In some embodiments, the appropriate reagent is chloromethyl methyl ether. In some embodiments, the appropriate base is potassium carbonate. In some embodiments, the appropriate solvent is acetone. In some embodiments, the appropriate temperature is room temperature and the appropriate amount of time stirred is about 15 hours (overnight).

In some embodiments, the protecting group is a benzyl protecting group. In some embodiments, appropriate conditions to install a benzyl protecting group include using an appropriate reagent and an appropriate base in an appropriate solvent at an appropriate temperature and amount of time. In some embodiments, the appropriate reagent is benzyl bromide. In some embodiments, the appropriate base is potassium carbonate. In some embodiments, the appropriate solvent is DMF. In some embodiments, the appropriate temperature is room temperature and the appropriate amount of time stirred is about 15 hours (overnight).

In some embodiments, intermediate I-4 is reacted under appropriate alkylation reaction conditions to provide intermediate I-5. In some embodiments, appropriate alkylation reaction conditions include using an appropriate reagent and an appropriate base in an appropriate solvent at an appropriate time and at an appropriate temperature. In some embodiments, the appropriate reagent is methyl iodide. In some embodiments, the appropriate base is potassium carbonate. In some embodiments, the appropriate solvent is DMF. In some embodiments, the appropriate temperature is 0° C. to 60° C. and the appropriate amount of time stirred is about 15 hours (overnight).

In some embodiments, intermediate I-5 is reacted under appropriate hydrolysis reaction conditions to provide intermediate I-3, followed by appropriate reduction reaction conditions to provide intermediate I-6. In some embodiments, appropriate hydrolysis reaction conditions include using an appropriate base in an appropriate solvent at an appropriate time and at an appropriate temperature. In some embodiments, the appropriate base is sodium hydroxide. In some embodiments, the appropriate solvent mixture is 2:1 ethanol/water. In some embodiments, the appropriate temperature is 80° C. and the appropriate amount of time stirred is about 15 hours (overnight).

In some embodiments, appropriate reduction reaction conditions include using an appropriate reagent in an appropriate solvent at an appropriate time and at an appropriate temperature. In some embodiments, the appropriate reagent is borane dimethylsulfide. In some embodiments, the appropriate solvent is THF. In some embodiments, the appropriate temperature is 0° C. to 80° C. and the appropriate amount of time stirred is about 2 hours.

In some embodiments, intermediate I-5 is reacted under appropriate reduction reaction conditions to provide intermediate I-6. In some embodiments, appropriate reduction reaction conditions include using an appropriate reagent in an appropriate solvent at an appropriate time and at an appropriate temperature. In some embodiments, the appropriate reagent is lithium aluminum hydride. In some embodiments, the appropriate solvent is THF. In some embodiments, the appropriate temperature is 0° C. to room temperature and the appropriate amount of time stirred is about 1 to 3 hours.

In some embodiments, intermediate I-6 is reacted under appropriate oxidation reaction conditions to provide intermediate I-7. In some embodiments, appropriate oxidation reaction conditions include using appropriate reagents in an appropriate solvent at an appropriate time and at an appropriate temperature. In some embodiments, appropriate reagents are pyridinium chlorochromate and silica gel or Celite®. In some embodiments, the appropriate solvent is DCM. In some embodiments, the appropriate temperature is room temperature and the appropriate amount of time stirred is about 2 to 3 hours.

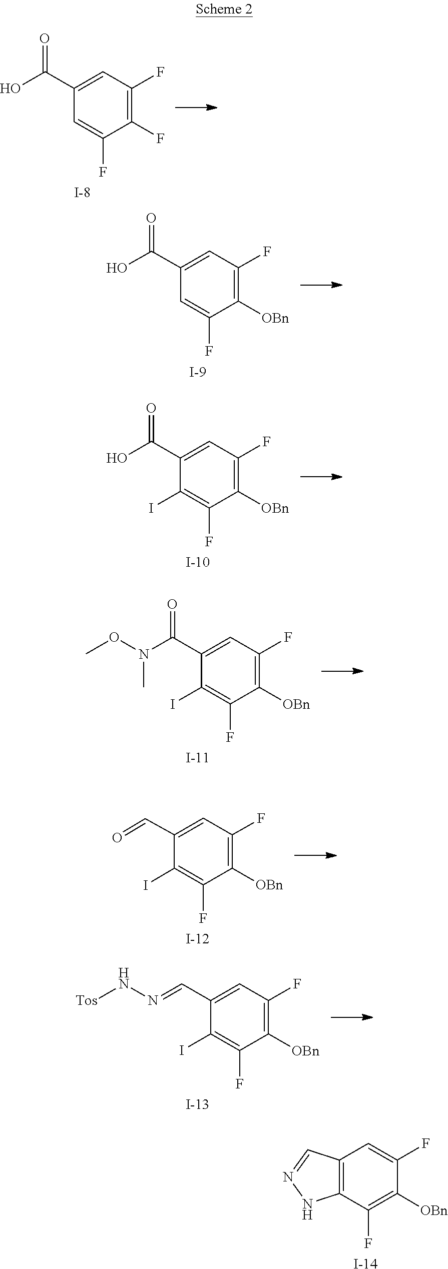

In some embodiments, compounds described herein are prepared as outlined in Scheme 2.

In some embodiments, intermediate I-8 is reacted under appropriate nucleophilic aromatic substitution reaction conditions to provide intermediate I-9. In some embodiments, appropriate nucleophilic aromatic substitution reaction conditions include using an appropriate reagent and an appropriate base in an appropriate solvent at an appropriate time and at an appropriate temperature. In some embodiments, the appropriate reagent is benzyl alcohol. In some embodiments, the appropriate base is sodium hydride. In some embodiments, the appropriate solvent is DMF. In some embodiments, the appropriate temperature is 0° C. to room temperature and the appropriate amount of time stirred is about 15 hours (overnight).

In some embodiments, intermediate I-9 is reacted under appropriate iodination reaction conditions to provide intermediate I-10. In some embodiments, appropriate iodination reaction conditions include using an appropriate reagent and an appropriate base in an appropriate solvent at an appropriate time and at an appropriate temperature. In some embodiments, the appropriate reagent is iodine. In some embodiments, the appropriate base is n-butyllithium. In some embodiments, the appropriate solvent is THF. In some embodiments, the appropriate temperature is −78° C. and the appropriate amount of time stirred is about 15 hours (overnight).

In some embodiments, intermediate I-10 is reacted under appropriate amide coupling reaction conditions to provide intermediate I-11. In some embodiments, appropriate amide coupling reaction conditions include using appropriate reagents in an appropriate solvent at an appropriate time and at an appropriate temperature. In some embodiments, the appropriate reagents are NO-dimethylhydroxylamine hydrochloride and 2,4,6-tripropyl-1,3,5,2,4,6-trioxatriphosphinane 2,4,6-trioxide. In some embodiments, the appropriate solvent is DCM. In some embodiments, the appropriate temperature is room temperature and the appropriate amount of time stirred is about 15 hours (overnight).

In some embodiments, intermediate I-11 is reacted under appropriate reduction reaction conditions to provide intermediate I-12. In some embodiments, appropriate reduction reaction conditions include using an appropriate reagent in an appropriate solvent at an appropriate time and at an appropriate temperature. In some embodiments, the appropriate reagent is diisobutylaluminum hydride. In some embodiments, the appropriate solvent is DCM. In some embodiments, the appropriate temperature is −78° C. and the appropriate amount of time stirred is about 2 hours.

In some embodiments, intermediate I-12 is reacted under appropriate hydrazone formation reaction conditions to provide intermediate I-13. In some embodiments, appropriate hydrazone formation reaction conditions include using an appropriate reagent and an appropriate acid in an appropriate solvent at an appropriate time and at an appropriate temperature. In some embodiments, the appropriate reagent is 4-methylbenzenesulfonohydrazide. In some embodiments, the appropriate acid is concentrated hydrochloric acid. In some embodiments, the appropriate solvent is ethanol. In some embodiments, the appropriate temperature is 50° C. and the appropriate amount of time stirred is about 15 hours (overnight).

In some embodiments, intermediate I-13 is reacted under appropriate indazole formation reaction conditions to provide intermediate I-14. In some embodiments, appropriate indazole formation reaction conditions include using an appropriate reagent in an appropriate solvent at an appropriate time and at an appropriate temperature. In some embodiments, the appropriate reagent is cuprous oxide. In some embodiments, the appropriate solvent is 3-methylbutan-1-ol. In some embodiments, the appropriate temperature is 130° C. and the appropriate amount of time stirred is about 15 hours (overnight).

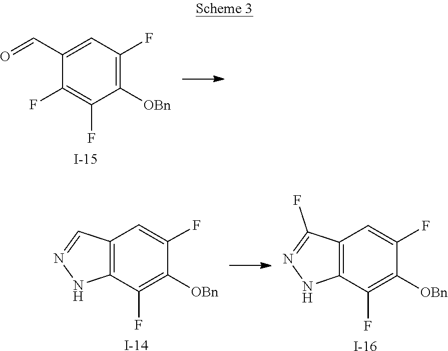

In some embodiments, compounds described herein are prepared as outlined in Scheme 3.

In some embodiments, intermediate I-15 is reacted under appropriate indazole formation reaction conditions to provide intermediate I-14. In some embodiments, appropriate indazole formation reaction conditions include using an appropriate reagent in an appropriate solvent at an appropriate time and at an appropriate temperature. In some embodiments, the appropriate reagent is hydrazine hydrate. In some embodiments, the appropriate solvent is DME. In some embodiments, the appropriate temperature is room temperature to 105° C. and the appropriate amount of time stirred is about 16 hours.

In some embodiments, intermediate I-14 is reacted under appropriate fluorination reaction conditions to provide intermediate I-16. In some embodiments, appropriate fluorination reaction conditions include using an appropriate reagent in an appropriate solvent at an appropriate time and at an appropriate temperature. In some embodiments, the appropriate reagent is 1-chloromethyl-4-fluoro-1,4-diazoniabicyclo[2.2.2]octane bis(tetrafluoroborate). In some embodiments, the appropriate solvent is MeCN. In some embodiments, the appropriate temperature is 90° C. and the appropriate amount of time stirred is about 12 hours.

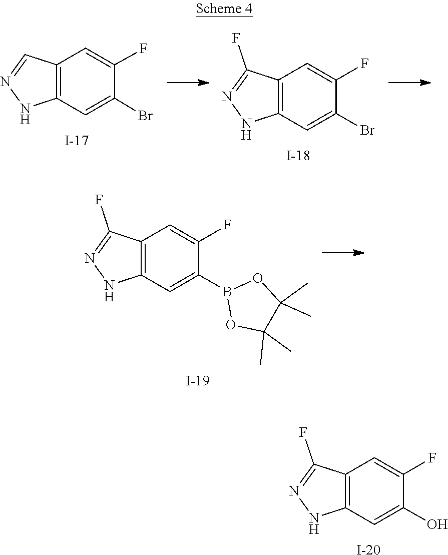

In some embodiments, compounds described herein are prepared as outlined in Scheme 4.

In some embodiments, intermediate I-17 is reacted under appropriate fluorination reaction conditions to provide intermediate I-18. In some embodiments, appropriate fluorination reaction conditions include using an appropriate reagent in an appropriate solvent at an appropriate time and at an appropriate temperature. In some embodiments, the appropriate reagent is 1-chloromethyl-4-fluoro-1,4-diazoniabicyclo[2.2.2]octane bis(tetrafluoroborate). In some embodiments, the appropriate solvent is MeCN. In some embodiments, the appropriate temperature is 90° C. and the appropriate amount of time stirred is about 4 hours.

In some embodiments, intermediate I-18 is reacted under appropriate Miyaura borylation reaction conditions to provide intermediate I-19. In some embodiments, appropriate Miyaura borylation reaction conditions include using an appropriate reagent, an appropriate catalyst, and an appropriate base in an appropriate solvent at an appropriate time and at an appropriate temperature. In some embodiments, the appropriate reagent is bis(pinacolato)diboron. In some embodiments, the appropriate catalyst is [1,1′-bis(diphenylphosphino)ferrocene]dichloropalladium (II). In some embodiments, the appropriate base is potassium acetate. In some embodiments, the appropriate solvent is dioxane. In some embodiments, the appropriate temperature is 105° C. and the appropriate amount of time stirred is about 15 hours (overnight).

In some embodiments, intermediate I-19 is reacted under appropriate oxidation reaction conditions to provide intermediate I-20. In some embodiments, appropriate oxidation reaction conditions include using an appropriate reagent and an appropriate base in an appropriate solvent at an appropriate time and at an appropriate temperature. In some embodiments, the appropriate reagent is hydrogen peroxide. In some embodiments, the appropriate base is sodium hydroxide. In some embodiments, the appropriate solvent is THF. In some embodiments, the appropriate temperature is 0° C. and the appropriate amount of time stirred is about 1 hour.

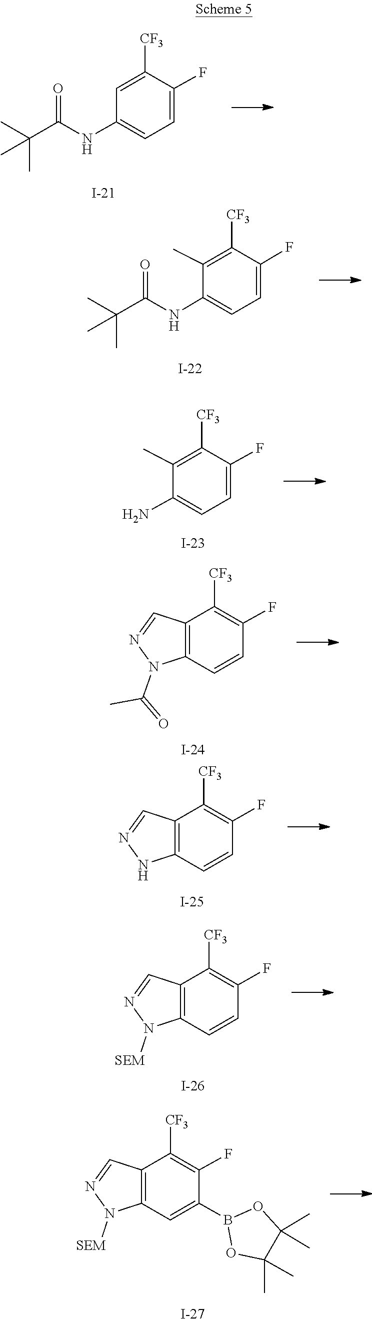

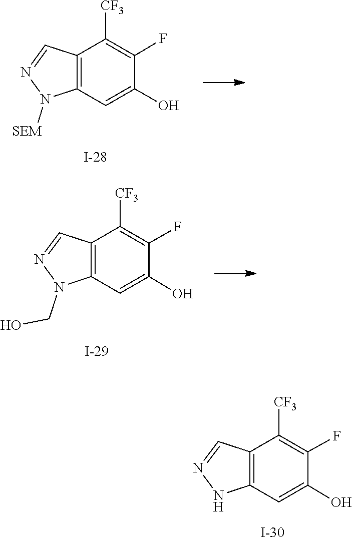

In some embodiments, compounds described herein are prepared as outlined in Scheme 5.

In some embodiments, intermediate I-21 is reacted under appropriate alkylation reaction conditions to provide intermediate I-22. In some embodiments, appropriate alkylation reaction conditions include using an appropriate reagent and an appropriate base in an appropriate solvent at an appropriate time and at an appropriate temperature. In some embodiments, the appropriate reagent is methyl iodide. In some embodiments, the appropriate base is n-butyllithium. In some embodiments, the appropriate solvent is THF. In some embodiments, the appropriate temperature is 0° C. and the appropriate amount of time stirred is about 3 hours.

In some embodiments, intermediate I-22 is reacted under appropriate hydrolysis reaction conditions to provide intermediate I-23. In some embodiments, appropriate hydrolysis reaction conditions include using appropriate acids in an appropriate solvent at an appropriate time and at an appropriate temperature. In some embodiments, the appropriate acid is hydrochloric acid. In some embodiments, the appropriate solvent is acetic acid. In some embodiments, the appropriate temperature is 118° C. and the appropriate amount of time stirred is about 15 hours (overnight).

In some embodiments, intermediate I-23 is reacted under appropriate indazole formation reaction conditions to provide intermediate I-24. In some embodiments, appropriate indazole formation reaction conditions include using appropriate reagents and an appropriate base in an appropriate solvent at an appropriate time and at an appropriate temperature. In some embodiments, the appropriate reagents are acetic anhydride, 18-crown-6, and isopentyl nitrite. In some embodiments, the appropriate base is potassium acetate. In some embodiments, the appropriate solvent is chloroform. In some embodiments, the appropriate temperature is 0° C. to 85° C. and the appropriate amount of time stirred is about 15 hours (overnight).

In some embodiments, intermediate I-24 is reacted under appropriate hydrolysis reaction conditions to provide intermediate I-25. In some embodiments, appropriate hydrolysis reaction conditions include using an appropriate acid in an appropriate solvent at an appropriate time and at an appropriate temperature. In some embodiments, the appropriate acid is hydrochloric acid. In some embodiments, the appropriate solvent is methanol. In some embodiments, the appropriate temperature is 95° C. and the appropriate amount of time stirred is about 2 hours.

In some embodiments, intermediate I-25 is reacted under appropriate alkylation reaction conditions to provide intermediate I-26. In some embodiments, appropriate alkylation reaction conditions include using an appropriate reagent and an appropriate base in an appropriate solvent at an appropriate time and at an appropriate temperature. In some embodiments, the appropriate reagent is 2-(trimethylsilyl)ethoxymethyl chloride. In some embodiments, the appropriate base is sodium hydride. In some embodiments, the appropriate solvent is DMF. In some embodiments, the appropriate temperature is 0° C. to room temperature and the appropriate amount of time stirred is about 15 hours (overnight).

In some embodiments, intermediate I-26 is reacted under appropriate borylation reaction conditions to provide intermediate I-27. In some embodiments, appropriate borylation reaction conditions include using appropriate reagents and an appropriate catalyst in an appropriate solvent at an appropriate time and at an appropriate temperature. In some embodiments, the appropriate reagents are 4-tert-butyl-2-(4-tert-butyl-2-pyridyl)pyridine and bis(pinacolato)diboron. In some embodiments, the appropriate catalyst is (1,5-cyclooctadiene)(methoxy)iridium(I) dimer. In some embodiments, the appropriate solvent is THF. In some embodiments, the appropriate temperature is 80° C. and the appropriate amount of time stirred is about 2 hours.

In some embodiments, intermediate I-27 is reacted under appropriate oxidation reaction conditions to provide intermediate I-28. In some embodiments, appropriate oxidation reaction conditions include using an appropriate reagent in an appropriate solvent at an appropriate time and at an appropriate temperature. In some embodiments, the appropriate reagent is sodium perborate tetrahydrate. In some embodiments, the appropriate solvent mixture is 2:1 THF/methanol. In some embodiments, the appropriate temperature is room temperature and the appropriate amount of time stirred is about 15 hours (overnight).

In some embodiments, intermediate I-28 is reacted under appropriate desilylation reaction conditions to provide intermediate I-29. In some embodiments, appropriate desilylation reaction conditions include using an appropriate acid in an appropriate solvent at an appropriate time and at an appropriate temperature. In some embodiments, the appropriate acid is trifluoroacetic acid. In some embodiments, the appropriate solvent is DCE. In some embodiments, the appropriate temperature is room temperature and the appropriate amount of time stirred is about 15 hours (overnight).

In some embodiments, intermediate I-29 is reacted under appropriate dealkylation reaction conditions to provide intermediate I-30. In some embodiments, appropriate dealkylation reaction conditions include using an appropriate base in an appropriate solvent at an appropriate time and at an appropriate temperature. In some embodiments, the appropriate base is ethylenediamine. In some embodiments, the appropriate solvent is ethanol. In some embodiments, the appropriate temperature is room temperature and the appropriate amount of time stirred is about 15 hours (overnight).

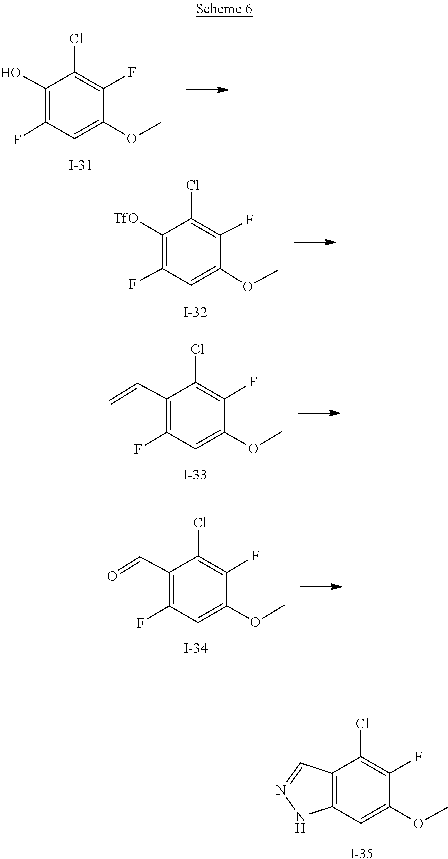

In some embodiments, compounds described herein are prepared as outlined in Scheme 6.

In some embodiments, intermediate I-31 is reacted under appropriate sulfonylation reaction conditions to provide intermediate I-32. In some embodiments, appropriate sulfonylation reaction conditions include using an appropriate reagent and an appropriate base in an appropriate solvent at an appropriate time and at an appropriate temperature. In some embodiments, the appropriate reagent is trifluoromethanesulfonic anhydride. In some embodiments, the appropriate base is pyridine. In some embodiments, the appropriate solvent is DCM. In some embodiments, the appropriate temperature is 0° C. to room temperature and the appropriate amount of time stirred is about 3 hours.

In some embodiments, intermediate I-32 is reacted under appropriate Suzuki coupling reaction conditions to provide intermediate I-33. In some embodiments, appropriate Suzuki coupling reaction conditions include using an appropriate reagent, an appropriate catalyst, and an appropriate base in an appropriate solvent at an appropriate time and at an appropriate temperature. In some embodiments, the appropriate reagent is potassium vinyltrifluoroborate. In some embodiments, the appropriate catalyst is [1,1′-bis(diphenylphosphino)ferrocene]dichloropalladium (II). In some embodiments, the appropriate base is triethylamine. In some embodiments, the appropriate solvent is ethanol. In some embodiments, the appropriate temperature is 80° C. and the appropriate amount of time stirred is about 15 hours (overnight).

In some embodiments, intermediate I-33 is reacted under appropriate ozonolysis reaction conditions to provide intermediate I-34. In some embodiments, appropriate ozonolysis reaction conditions include using an appropriate reagent and an appropriate base in an appropriate solvent at an appropriate time and at an appropriate temperature. In some embodiments, the appropriate reagent is ozone. In some embodiments, the appropriate base is triphenylphosphine. In some embodiments, the appropriate solvent is DCM. In some embodiments, the appropriate temperature is −78° C. to room temperature and the appropriate amount of time stirred is about 15 hours (overnight).

In some embodiments, intermediate I-34 is reacted under appropriate indazole formation reaction conditions to provide intermediate I-35. In some embodiments, appropriate indazole formation reaction conditions include using an appropriate reagent in an appropriate solvent at an appropriate time and at an appropriate temperature. In some embodiments, the appropriate reagent is hydrazine hydrate. In some embodiments, the appropriate solvent is NMP. In some embodiments, the appropriate temperature is 130° C. and the appropriate amount of time stirred is about 3 hours.

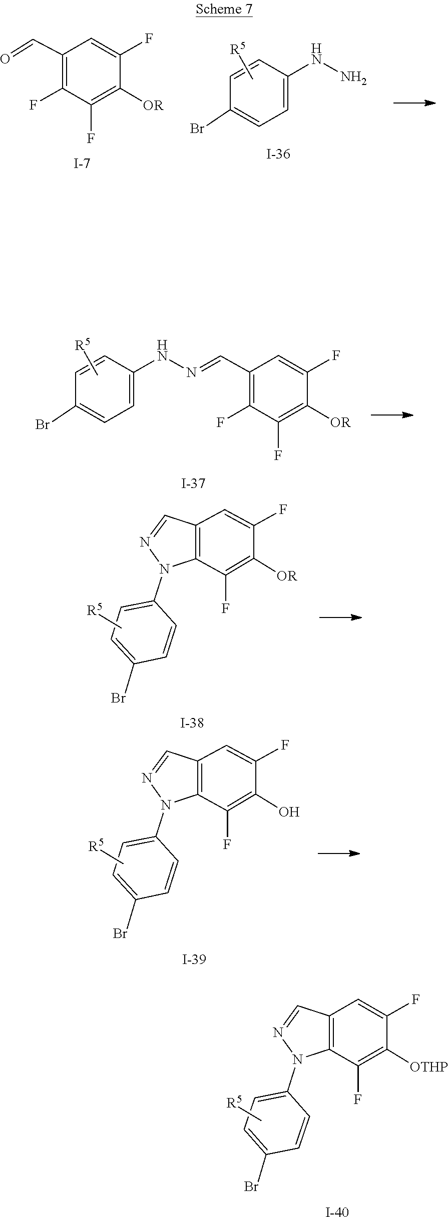

In some embodiments, compounds described herein are prepared as outlined in Scheme 7.

In some embodiments, intermediate I-7 is reacted under appropriate hydrazone formation reaction conditions to provide intermediate I-37. In some embodiments, appropriate hydrazone formation reaction conditions include using an appropriate reagent in an appropriate solvent at an appropriate time and at an appropriate temperature. In some embodiments, the appropriate reagent is intermediate I-36. In some embodiments, the appropriate solvent is dioxane. In some embodiments, the appropriate solvent is methanol. In some embodiments, the appropriate solvent is ethanol. In some embodiments, the appropriate temperature is 80° C. to 100° C. and the appropriate amount of time stirred is about 1 hour to 17 hours. In some embodiments, the appropriate temperature is room temperature and the appropriate amount of time stirred is about 5 minutes to 3 hours.

In some embodiments, intermediate I-37 is reacted under appropriate indazole formation reaction conditions to provide intermediate I-38. In some embodiments, appropriate indazole formation reaction conditions include using an appropriate base in an appropriate solvent at an appropriate time and at an appropriate temperature. In some embodiments, the appropriate base is potassium carbonate. In some embodiments, the appropriate base is potassium tert-butoxide. In some embodiments, the appropriate solvent is NMP. In some embodiments, the appropriate solvent is 2-methyl-THF. In some embodiments, the appropriate temperature is 160° C. to 180° C. and the appropriate amount of time stirred is about 1 hour to 2 hours. In some embodiments, the appropriate temperature is 210° C. to 220° C. and the appropriate amount of time stirred is about 3 to 30 minutes. In some embodiments, the appropriate temperature is 90° C. and the appropriate amount of time stirred is about 8 hours.

In some embodiments, intermediate I-38 is reacted under appropriate demethylation reaction conditions to provide intermediate I-39. In some embodiments, appropriate demethylation reaction conditions include using an appropriate reagent in an appropriate solvent at an appropriate time and at an appropriate temperature. In some embodiments, the appropriate reagent is pyridinium hydrochloride. In some embodiments, the appropriate reagent is boron tribromide. In some embodiments, the appropriate solvent is DCM. In some embodiments, the appropriate temperature is 170° C. to 180° C. and the appropriate amount of time stirred is about 1 hour to 4 hours. In some embodiments, the appropriate temperature is −78° C. and the appropriate amount of time stirred is about 6 hours. In some embodiments, the appropriate temperature is 35° C. and the appropriate amount of time stirred is about 45 hours.

In some embodiments, intermediate I-39 is reacted under appropriate alkylation reaction conditions to provide intermediate I-40. In some embodiments, appropriate alkylation reaction conditions include using appropriate reagents in an appropriate solvent at an appropriate time and at an appropriate temperature. In some embodiments, the appropriate reagents are pyridinium para-toluenesulfonate and dihydropyran. In some embodiments, the appropriate solvent is DCM. In some embodiments, the appropriate temperature is 0° C. to room temperature and the appropriate amount of time stirred is about 16.5 hours.

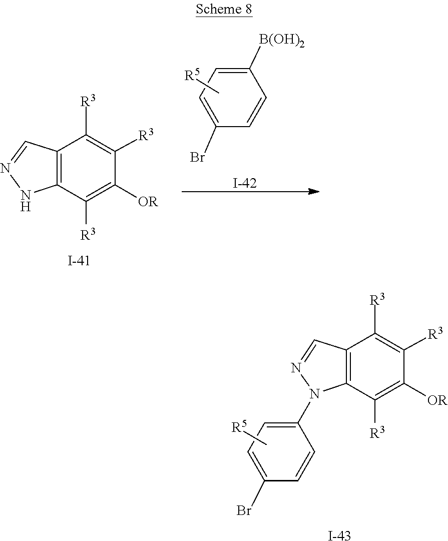

In some embodiments, compounds described herein are prepared as outlined in Scheme 8.

In some embodiments, intermediate I-41 is reacted under appropriate Chan-Lam coupling reaction conditions to provide intermediate I-43. In some embodiments, appropriate Chan-Lam coupling reaction conditions include using an appropriate reagent, an appropriate catalyst, and an appropriate base in an appropriate solvent at an appropriate time and at an appropriate temperature. In some embodiments, the appropriate reagent is intermediate I-42. In some embodiments, the appropriate catalyst is cupric acetate. In some embodiments, the appropriate base is pyridine. In some embodiments, the appropriate solvent is DCM. In some embodiments, the appropriate solvent is DCE. In some embodiments, the appropriate solvent is DMF. In some embodiments, the appropriate temperature is room temperature and the appropriate amount of time stirred under an oxygen atmosphere at an appropriate pressure is about 16 hours to 64 hours. In some embodiments, the appropriate temperature is 80° C. and the appropriate amount of time stirred under an oxygen atmosphere at an appropriate pressure is about 15 hours (overnight). In some embodiments, the appropriate temperature is room temperature to 110° C. and the appropriate amount of time stirred under an oxygen atmosphere at an appropriate pressure is about 105 hours. In some embodiments, the appropriate pressure of oxygen is 15 psi.

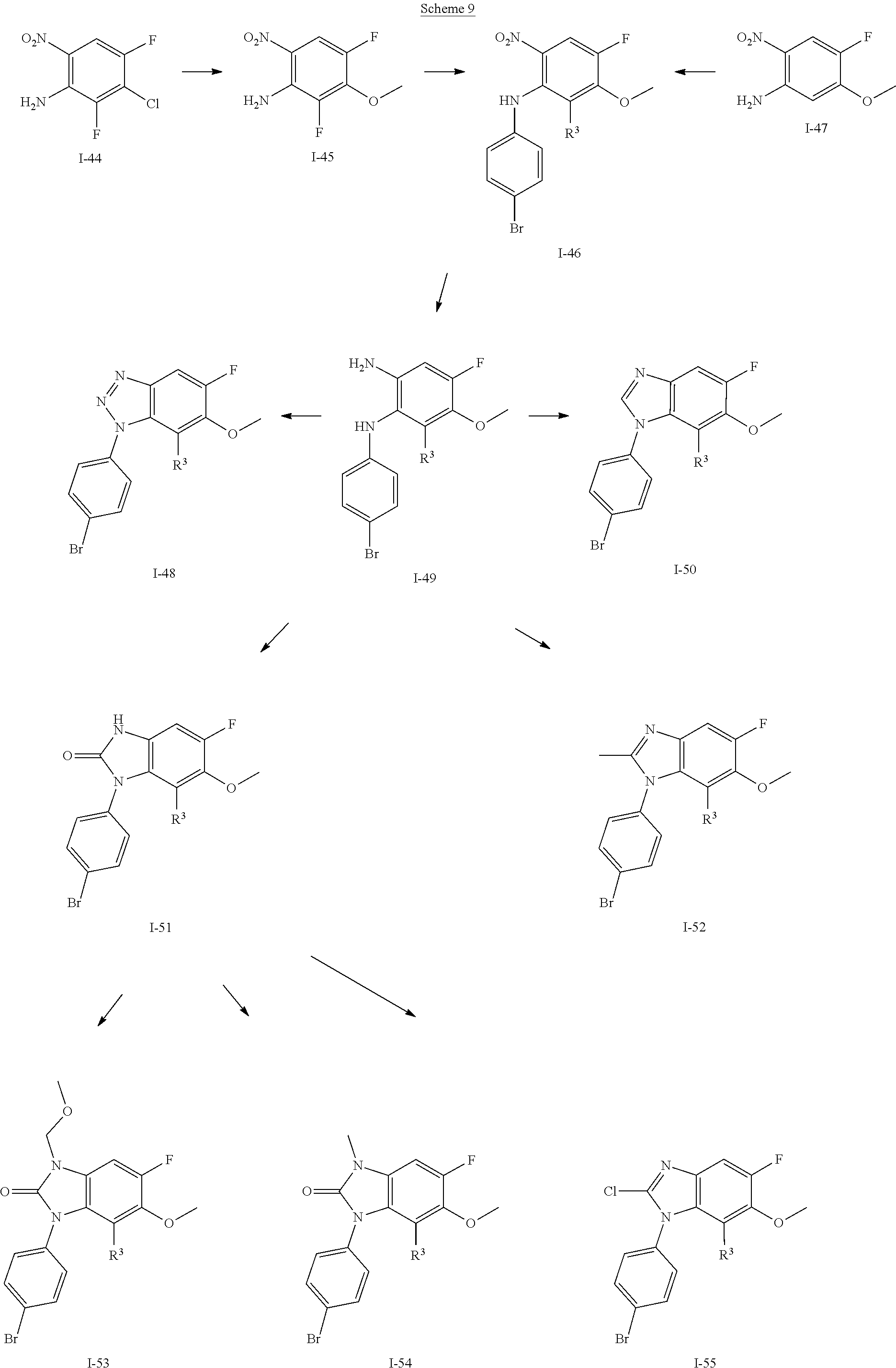

In some embodiments, compounds described herein are prepared as outlined in Scheme 9.

In some embodiments, intermediate I-44 is reacted under appropriate nucleophilic aromatic substitution reaction conditions to provide intermediate I-45. In some embodiments, appropriate nucleophilic aromatic substitution reaction conditions include using an appropriate base in an appropriate solvent at an appropriate time and at an appropriate temperature. In some embodiments, the appropriate base is sodium methoxide. In some embodiments, the appropriate solvent is methanol. In some embodiments, the appropriate temperature is 90° C. and the appropriate amount of time stirred is about 2 hours.

In some embodiments, intermediate I-45 or intermediate I-47 is reacted under appropriate Chan-Lam coupling reaction conditions to provide intermediate I-46. In some embodiments, appropriate Chan-Lam coupling reaction conditions include using an appropriate reagent, an appropriate catalyst, and an appropriate base in an appropriate solvent at an appropriate time and at an appropriate temperature. In some embodiments, the appropriate reagent is (4-bromophenyl)boronic acid. In some embodiments, the appropriate catalyst is cupric acetate. In some embodiments, the appropriate base is triethylamine. In some embodiments, the appropriate solvent is DCM. In some embodiments, the appropriate temperature is room temperature and the appropriate amount of time stirred is about 21 hours.

In some embodiments, intermediate I-46 is reacted under appropriate reduction reaction conditions to provide intermediate I-49. In some embodiments, appropriate reduction reaction conditions include using an appropriate reagent in an appropriate solvent at an appropriate time and at an appropriate temperature. In some embodiments, the appropriate reagent is stannous chloride dihydrate. In some embodiments, the appropriate solvent is ethanol. In some embodiments, the appropriate temperature is 70° C. and the appropriate amount of time stirred is about 2 hours.

In some embodiments, intermediate I-49 is reacted under appropriate benzotriazole formation reaction conditions to provide intermediate I-48. In some embodiments, appropriate benzotriazole formation reaction conditions include using an appropriate reagent and an appropriate acid in an appropriate solvent at an appropriate time and at an appropriate temperature. In some embodiments, the appropriate reagent is sodium nitrite. In some embodiments, the appropriate acid is concentrated sulfuric acid. In some embodiments, the appropriate solvent mixture is THF/water. In some embodiments, the appropriate temperature is 0° C. and the appropriate amount of time stirred is about 15 minutes.

In some embodiments, intermediate I-49 is reacted under appropriate benzimidazole formation reaction conditions to provide intermediate I-50. In some embodiments, appropriate benzimidazole formation reaction conditions include using an appropriate reagent in an appropriate solvent at an appropriate time and at an appropriate temperature. In some embodiments, the appropriate reagent is formic acid. In some embodiments, the appropriate solvent mixture is dioxane/water. In some embodiments, the appropriate temperature is 100° C. and the appropriate amount of time stirred is about 3 hours.

In some embodiments, intermediate I-49 is reacted under appropriate benzimidazole formation reaction conditions to provide intermediate I-52. In some embodiments, appropriate benzimidazole formation reaction conditions include using an appropriate reagent in an appropriate solvent at an appropriate time and at an appropriate temperature. In some embodiments, the appropriate reagent is acetyl chloride. In some embodiments, the appropriate solvent is toluene. In some embodiments, the appropriate temperature is 0° C. to 115° C. and the appropriate amount of time stirred is about 5 hours.

In some embodiments, intermediate I-49 is reacted under appropriate urea formation reaction conditions to provide intermediate I-51. In some embodiments, appropriate urea formation reaction conditions include using an appropriate reagent and an appropriate base in an appropriate solvent at an appropriate time and at an appropriate temperature. In some embodiments, the appropriate reagent is 1,1′-carbonyldiimidazole. In some embodiments, the appropriate base is pyridine. In some embodiments, the appropriate solvent is THF. In some embodiments, the appropriate temperature is 65° C. and the appropriate amount of time stirred is about 1 hour.

In some embodiments, intermediate I-51 is reacted under appropriate alkylation reaction conditions to provide intermediate I-53. In some embodiments, appropriate alkylation reaction conditions include using an appropriate reagent and an appropriate base in an appropriate solvent at an appropriate time and at an appropriate temperature. In some embodiments, the appropriate reagent is chloromethyl methyl ether. In some embodiments, the appropriate base is sodium hydride. In some embodiments, the appropriate solvent is DMF. In some embodiments, the appropriate temperature is room temperature and the appropriate amount of time stirred is about 2.5 hours.

In some embodiments, intermediate I-51 is reacted under appropriate alkylation reaction conditions to provide intermediate I-54. In some embodiments, appropriate alkylation reaction conditions include using an appropriate reagent and an appropriate base in an appropriate solvent at an appropriate time and at an appropriate temperature. In some embodiments, the appropriate reagent is methyl iodide. In some embodiments, the appropriate base is cesium carbonate. In some embodiments, the appropriate solvent is DMF.

In some embodiments, the appropriate temperature is room temperature and the appropriate amount of time stirred is about 30 minutes.

In some embodiments, intermediate I-51 is reacted under appropriate chlorination reaction conditions to provide intermediate I-55. In some embodiments, appropriate chlorination reaction conditions include using an appropriate reagent at an appropriate time and at an appropriate temperature. In some embodiments, the appropriate reagent is phosphoryl chloride. In some embodiments, the appropriate temperature is 100° C. and the appropriate amount of time stirred is about 5 hours.

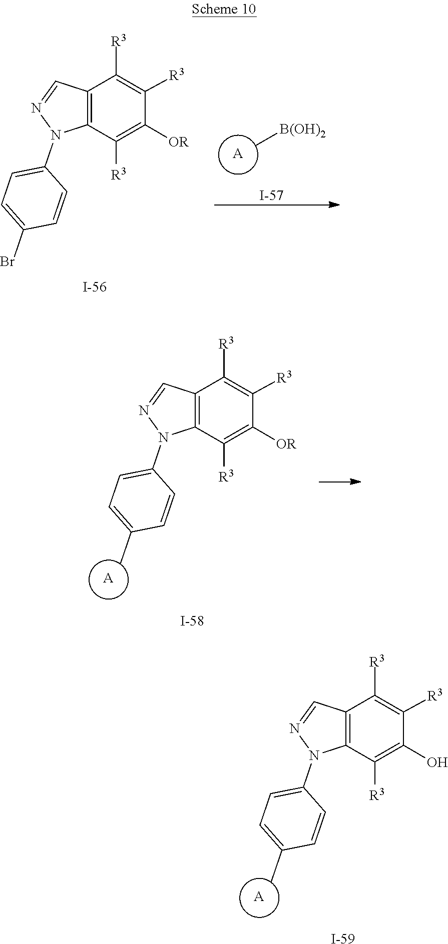

In some embodiments, compounds described herein are prepared as outlined in Scheme 10.

In some embodiments, intermediate I-56 is reacted under appropriate Suzuki coupling reaction conditions to provide intermediate I-58, followed by removal of an appropriate protecting group to provide intermediate I-59. In some embodiments, appropriate Suzuki coupling reaction conditions include using an appropriate reagent, an appropriate catalyst, and an appropriate base in an appropriate solvent at an appropriate time and at an appropriate temperature. In some embodiments, the appropriate reagent is intermediate I-57.

In some embodiments, the appropriate catalyst is [1,1′-bis(diphenylphosphino)ferrocene]dichloropalladium (II). In some embodiments, the appropriate catalyst is bis(triphenylphosphine)palladium dichloride. In some embodiments, the appropriate catalyst is tetrakis(triphenylphosphine)palladium(0). In some embodiments, the appropriate catalyst is tris(dibenzylideneacetone)dipalladium(0). In some embodiments, the appropriate catalyst ligand is tricyclohexylphopshine. In some embodiments, the appropriate catalyst ligand is XPhos. In some embodiments, the appropriate base is potassium carbonate. In some embodiments, the appropriate base is sodium carbonate. In some embodiments, the appropriate base is cesium carbonate. In some embodiments, the appropriate base is potassium phosphate. In some embodiments, the appropriate base is potassium acetate. In some embodiments, the appropriate solvent mixture is DME/water. In some embodiments, the appropriate solvent mixture is DMF/water. In some embodiments, the appropriate solvent is dioxane. In some embodiments, the appropriate solvent mixture is DME/ethanol. In some embodiments, the appropriate temperature is 80° C. and the appropriate amount of time stirred is about 1 to 2 hours. In some embodiments, the appropriate temperature is 80° C. to 100° C. and the appropriate amount of time stirred is about 0.5 to 67 hours. In some embodiments, the appropriate temperature is 100° C. and the appropriate amount of time stirred is about 20 minutes. In some embodiments, the appropriate temperature is 100° C. and the appropriate amount of time stirred is about 30 minutes. In some embodiments, the appropriate temperature is 80° C. to 100° C. and the appropriate amount of time stirred is about 0.5 to 2 hours. In some embodiments, the appropriate temperature is 80° C. and the appropriate amount of time stirred is about 18 hours. In some embodiments, the appropriate temperature is 80° C. to 100° C. and the appropriate amount of time stirred is about 3.5 to 18 hours. In some embodiments, the appropriate temperature is 110° C. and the appropriate amount of time stirred is about 15 hours (overnight).

In some embodiments, the protecting group is a benzyl protecting group. In some embodiments, appropriate conditions to remove a benzyl protecting group include using appropriate hydrogenation conditions using an appropriate catalyst in an appropriate solvent at an appropriate temperature and amount of time. In some embodiments, the appropriate catalyst is palladium on carbon. In some embodiments, the appropriate catalyst is platinum dioxide. In some embodiments, the appropriate solvent is methanol. In some embodiments, the appropriate solvent is ethyl acetate. In some embodiments, the appropriate solvent mixture is 1:1 methanol/ethyl acetate. In some embodiments, the appropriate temperature is room temperature and the appropriate amount of time stirred under a hydrogen atmosphere at an appropriate pressure is about 1 hour. In some embodiments, the appropriate temperature is room temperature and the appropriate amount of time stirred under a hydrogen atmosphere at an appropriate pressure is about 2 to 3 hours. In some embodiments, the appropriate pressure of hydrogen is atmospheric pressure.

In some embodiments, appropriate conditions to remove a benzyl protecting group include using an appropriate reagent in an appropriate solvent at an appropriate temperature and amount of time. In some embodiments, the appropriate reagent is boron tribromide. In some embodiments, the appropriate solvent is DCM. In some embodiments, the appropriate temperature is −78° C. to room temperature and the appropriate amount of time stirred is about 2 to 3 hours.

In some embodiments, the protecting group is a MOM protecting group. In some embodiments, appropriate conditions to remove a MOM protecting group include using an appropriate acid in an appropriate solvent at an appropriate temperature and amount of time. In some embodiments, the appropriate acid is trifluoroacetic acid. In some embodiments, the appropriate solvent is DCM. In some embodiments, the appropriate temperature is room temperature and the appropriate amount of time stirred is about 1 to 2 hours.

In some embodiments, the protecting group is a methyl protecting group. In some embodiments, appropriate conditions to remove a methyl protecting group include using an appropriate reagent in an appropriate solvent at an appropriate temperature and amount of time. In some embodiments, the appropriate reagent is boron tribromide. In some embodiments, the appropriate reagent is pyridinium hydrochloride. In some embodiments, the appropriate solvent is DCM. In some embodiments, the appropriate temperature is −78° C. to room temperature and the appropriate amount of time stirred is about 2 to 3 hours. In some embodiments, the appropriate temperature is −78° C. to 40° C. and the appropriate amount of time stirred is about 21 to 24 hours. In some embodiments, the appropriate temperature is 170° C. and the appropriate amount of time stirred is about 6 hours.

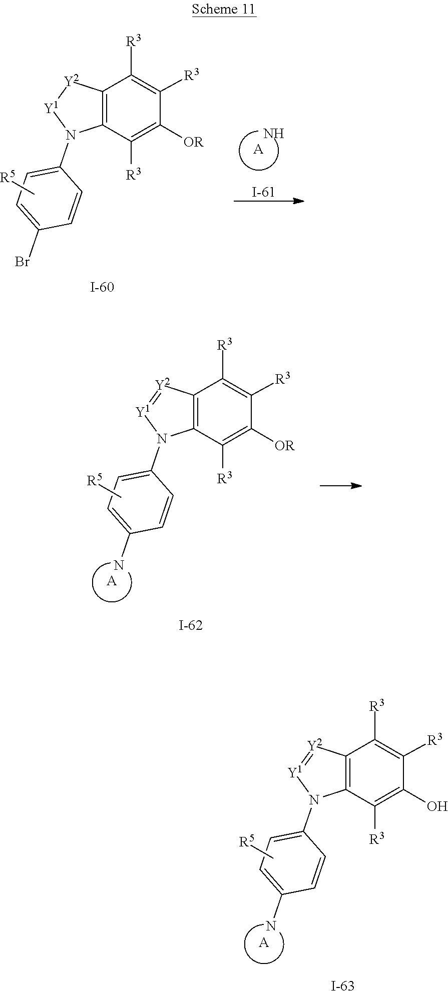

In some embodiments, compounds described herein are prepared as outlined in Scheme 11.

In some embodiments, intermediate I-60 is reacted under appropriate Buchwald-Hartwig coupling reaction conditions to provide intermediate I-62, followed by removal of an appropriate protecting group to provide intermediate I-63. In some embodiments, appropriate Buchwald-Hartwig coupling reaction conditions include using an appropriate reagent, an appropriate catalyst, and an appropriate base in an appropriate solvent at an appropriate time and at an appropriate temperature. In some embodiments, the appropriate reagent is Intermediate I-61. In some embodiments, the appropriate catalyst is tris(dibenzylideneacetone)dipalladium(0). In some embodiments, the appropriate catalyst is bis(tri-tert-butylphosphine)palladium(O). In some embodiments, the appropriate catalyst is palladium(II) acetate. In some embodiments, the appropriate catalyst ligand is RuPhos. In some embodiments, the appropriate catalyst ligand is XantPhos. In some embodiments, the appropriate catalyst ligand is XPhos. In some embodiments, the appropriate catalyst ligand is BINAP. In some embodiments, the appropriate catalyst ligand is tri-tert-butylphosphine. In some embodiments, the appropriate base is sodium tert-butoxide. In some embodiments, the appropriate base is cesium carbonate. In some embodiments, the appropriate solvent is toluene. In some embodiments, the appropriate solvent is dioxane. In some embodiments, the appropriate temperature is 100° C. and the appropriate amount of time stirred is about 1 hour. In some embodiments, the appropriate temperature is 80° C. to 100° C. and the appropriate amount of time stirred is about 0.5 to 90 hours.

In some embodiments, the protecting group is a benzyl protecting group. In some embodiments, appropriate conditions to remove a benzyl protecting group include using appropriate hydrogenation conditions using an appropriate catalyst in an appropriate solvent at an appropriate temperature and amount of time. In some embodiments, the appropriate catalyst is palladium on carbon. In some embodiments, the appropriate solvent is methanol. In some embodiments, the appropriate solvent is ethyl acetate. In some embodiments, the appropriate temperature is room temperature and the appropriate amount of time stirred under a hydrogen atmosphere at an appropriate pressure is about 2 to 5.5 hours. In some embodiments, the appropriate pressure of hydrogen is atmospheric pressure.

In some embodiments, the protecting group is a MOM protecting group. In some embodiments, appropriate conditions to remove a MOM protecting group include using an appropriate acid in an appropriate solvent at an appropriate temperature and amount of time. In some embodiments, the appropriate acid is trifluoroacetic acid. In some embodiments, the appropriate acid is hydrochloric acid. In some embodiments, the appropriate solvent is DCM. In some embodiments, the appropriate solvent mixture is 2:1 THF/methanol. In some embodiments, the appropriate temperature is room temperature and the appropriate amount of time stirred is about 0.5 to 2 hours. In some embodiments, the appropriate temperature is room temperature to 50° C. and the appropriate amount of time is about 66 hours.

In some embodiments, the protecting group is a THP protecting group. In some embodiments, appropriate conditions to remove a THP protecting group include using an appropriate acid in an appropriate solvent at an appropriate temperature and amount of time. In some embodiments, the appropriate acid is hydrochloric acid. In some embodiments, the appropriate solvent mixture is 2:1 THF/methanol. In some embodiments, the appropriate solvent mixture is 1:1 THF/methanol. In some embodiments, the appropriate solvent mixture is 1:2 THF/methanol. In some embodiments, the appropriate solvent mixture is 1:1 DCM/methanol. In some embodiments, the appropriate temperature is room temperature and the appropriate amount of time stirred is about 40 minutes to 2 hours. In some embodiments, the appropriate temperature is room temperature and the appropriate amount of time stirred is about 0.5 to 1 hour.

In some embodiments, the protecting group is a methyl protecting group. In some embodiments, appropriate conditions to remove a methyl protecting group include using an appropriate reagent in an appropriate solvent at an appropriate temperature and amount of time. In some embodiments, the appropriate reagent is boron tribromide. In some embodiments, the appropriate reagent is pyridinium hydrochloride. In some embodiments, the appropriate solvent is DCM. In some embodiments, the appropriate temperature is 0° C. to room temperature and the appropriate amount of time stirred is about 15 minutes to 3 hours. In some embodiments, the appropriate temperature is 40° C. to room temperature and the appropriate amount of time stirred is about 1 to 5 hours. In some embodiments, the appropriate temperature is 150° C. to 170° C. and the appropriate amount of time stirred is about 20 minutes to 4 hours.

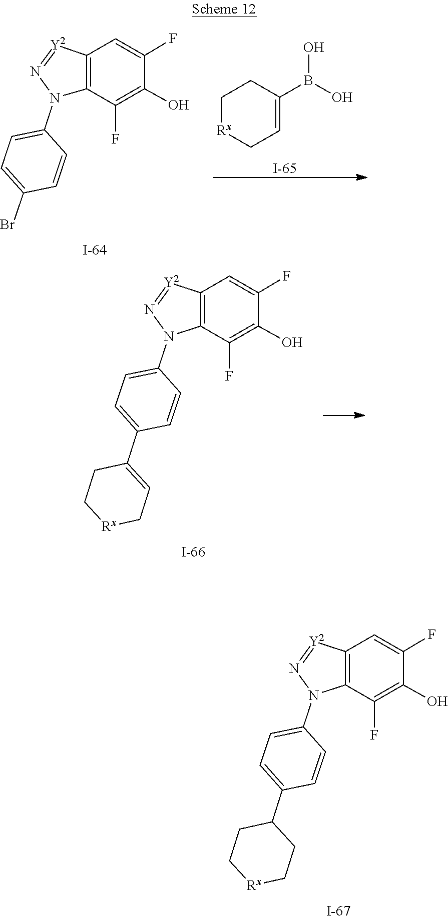

In some embodiments, compounds described herein are prepared as outlined in Scheme 12.

In some embodiments, intermediate I-64 is reacted under appropriate Suzuki coupling reaction conditions to provide intermediate I-66. In some embodiments, appropriate Suzuki coupling reaction conditions include using an appropriate reagent, an appropriate catalyst, and an appropriate base in an appropriate solvent at an appropriate time and at an appropriate temperature. In some embodiments, the appropriate reagent is intermediate I-65. In some embodiments, the appropriate reagent is intermediate I-65, wherein R^{x}═CH₂, C(CH₃)₂, O, N(SO₂Me), N(CH(CH₃)₂), or N(CH₃). In some embodiments, the appropriate catalyst is [1,1′-bis(diphenylphosphino)ferrocene]dichloropalladium (II). In some embodiments, the appropriate base is potassium carbonate. In some embodiments, the appropriate solvent mixture is DME/water. In some embodiments, the appropriate temperature is 80° C. and the appropriate amount of time stirred is about 2 hours.

In some embodiments, intermediate I-66 is reacted under appropriate hydrogenation reaction conditions to provide intermediate I-67. In some embodiments, appropriate hydrogenation reaction conditions include using an appropriate catalyst in an appropriate solvent at an appropriate time and at an appropriate temperature. In some embodiments, the appropriate catalyst is palladium on carbon. In some embodiments, the appropriate solvent is methanol. In some embodiments, the appropriate temperature is room temperature and the appropriate amount of time stirred under a hydrogen atmosphere at an appropriate pressure is about 17 to 93 hours. In some embodiments, the appropriate pressure of hydrogen is atmospheric pressure.

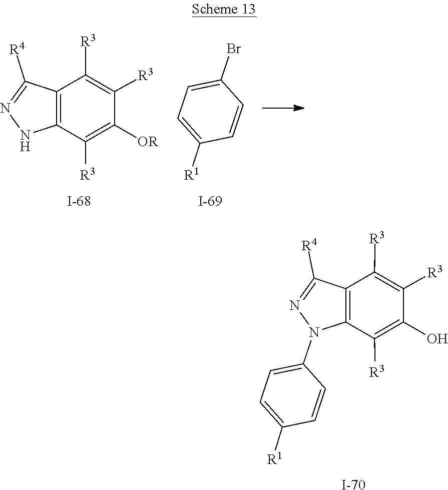

In some embodiments, compounds described herein are prepared as outlined in Scheme 13.

In some embodiments, intermediate I-68 is reacted under appropriate Buchwald-Hartwig coupling reaction conditions followed by removal of an appropriate protecting group to provide intermediate I-70. In some embodiments, appropriate Buchwald-Hartwig coupling reaction conditions include using an appropriate reagent, an appropriate catalyst, and an appropriate base in an appropriate solvent at an appropriate time and at an appropriate temperature. In some embodiments, the appropriate reagent is Intermediate I-69. In some embodiments, the appropriate catalyst is tris(dibenzylideneacetone)dipalladium(0). In some embodiments, the appropriate catalyst is Brettphos Pd G4. In some embodiments, the appropriate catalyst is cuprous iodide. In some embodiments, the appropriate catalyst ligand is tBuXPhos. In some embodiments, the appropriate catalyst ligand is trans-N,N′-dimethylcyclohexane-1,2-diamine. In some embodiments, the appropriate base is sodium tert-butoxide. In some embodiments, the appropriate base is potassium phosphate. In some embodiments, the appropriate solvent is toluene. In some embodiments, the appropriate solvent is DME. In some embodiments, the appropriate solvent is dioxane. In some embodiments, the appropriate temperature is 100° C. and the appropriate amount of time stirred is about 4 to 21 hours. In some embodiments, the appropriate temperature is 120° C. and the appropriate amount of time stirred is about 20 minutes. In some embodiments, the appropriate temperature is 85° C. and the appropriate amount of time stirred is about 15 hours (overnight). In some embodiments, the appropriate temperature is 100° C. and the appropriate amount of time stirred is about 15 hours (overnight). In some embodiments, the appropriate temperature is 110° C. and the appropriate amount of time stirred is about 10 hours.

In some embodiments, the protecting group is a benzyl protecting group. In some embodiments, appropriate conditions to remove a benzyl protecting group include using appropriate hydrogenation conditions using an appropriate catalyst in an appropriate solvent at an appropriate temperature and amount of time. In some embodiments, the appropriate catalyst is palladium on carbon. In some embodiments, the appropriate solvent is methanol. In some embodiments, the appropriate solvent is ethyl acetate. In some embodiments, the appropriate temperature is room temperature and the appropriate amount of time stirred under a hydrogen atmosphere at an appropriate pressure is about 2 to 22 hours. In some embodiments, the appropriate pressure of hydrogen is atmospheric pressure.

In some embodiments, the protecting group is a MOM protecting group. In some embodiments, appropriate conditions to remove a MOM protecting group include using an appropriate acid in an appropriate solvent at an appropriate temperature and amount of time. In some embodiments, the appropriate acid is trifluoroacetic acid. In some embodiments, the appropriate acid is hydrochloric acid. In some embodiments, the appropriate solvent is DCM. In some embodiments, the appropriate solvent mixture is 2:1 THF/methanol. In some embodiments, the appropriate temperature is room temperature and the appropriate amount of time stirred is about 0.5 to 2 hours. In some embodiments, the appropriate temperature is room temperature to 50° C. and the appropriate amount of time stirred is about 66 hours.

In some embodiments, the protecting group is a THP protecting group. In some embodiments, appropriate conditions to remove a THP protecting group include using an appropriate acid in an appropriate solvent at an appropriate temperature and amount of time. In some embodiments, the appropriate acid is hydrochloric acid. In some embodiments, the appropriate solvent mixture is 2:1 THF/methanol. In some embodiments, the appropriate solvent mixture is 1:1 THF/methanol. In some embodiments, the appropriate solvent mixture is 1:2 THF/methanol. In some embodiments, the appropriate solvent mixture is 1:1 DCM/methanol. In some embodiments, the appropriate temperature is room temperature and the appropriate amount of time stirred is about 40 minutes to 2 hours. In some embodiments, the appropriate temperature is room temperature and the appropriate amount of time stirred is about 0.5 to 1 hour.

In some embodiments, the protecting group is a methyl protecting group. In some embodiments, appropriate conditions to remove a methyl protecting group include using an appropriate reagent in an appropriate solvent at an appropriate temperature and amount of time. In some embodiments, the appropriate reagent is boron tribromide. In some embodiments, the appropriate reagent is pyridinium hydrochloride. In some embodiments, the appropriate solvent is DCM. In some embodiments, the appropriate temperature is −78° C. to room temperature and the appropriate amount of time stirred is about 18 to 22 hours. In some embodiments, the appropriate temperature is 0° C. to room temperature and the appropriate amount of time stirred is about 15 minutes to 3 hours. In some embodiments, the appropriate temperature is 40° C. to room temperature and the appropriate amount of time stirred is about 1 to 5 hours. In some embodiments, the appropriate temperature is 150° C. to 180° C. and the appropriate amount of time stirred is about 20 minutes to 4 hours.

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl group. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic group, wherein each of the atoms forming the ring (i.e. skeletal atoms) is a carbon atom. In some embodiments, cycloalkyls are spirocyclic or bridged compounds. In some embodiments, cycloalkyls are fully saturated. In some embodiments, cycloalkyls are partially unsaturated. In some embodiments, cycloalkyls are optionally fused with an aromatic ring, and the point of attachment is at a carbon that is not an aromatic ring carb on atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Poly cyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g., —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to a divalent heteroalkyl group.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, benzotriazole, purine, quinolizine, quinoline, isoquinoline, cinnoline, phthalazine, quinazoline, quinoxaline, 1,8-naphthyridine, and pteridine. In some embodiments, a heteroaryl contains 0-4N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, heterocycloalkyls are spirocyclic or bridged compounds. In some embodiments, heterocycloalkyls are fully saturated. In some embodiments, heterocycloalkyls are partially unsaturated. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-Cioheterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part oflarger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s). In some other embodiments, optional substituents are individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, —CH₂CO₂H, —CH₂CO₂alkyl, —CH₂C(═O)NH₂, —CH₂C(═O)NH(alkyl), —CH₂C(═O)N(alkyl)₂, —CH₂S(═O)₂NH₂, —CH₂S(═O)₂NH(alkyl), —CH₂S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream.

In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.0010% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, a compound disclosed herein is formulated to provide a controlled release of the compound. Controlled release refers to the release of the compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

Approaches to deliver the intact therapeutic compound to the particular regions of the gastrointestinal tract (e.g., such as the colon), include:
(i) Coating with polymers: The intact molecule can be delivered to the colon without absorbing at the upper part of the intestine by coating of the drug molecule with the suitable polymers, which degrade only in the colon.
(ii) Coating with pH-sensitive polymers: The majority of enteric and colon targeted delivery systems are based on the coating of tablets or pellets, which are filled into conventional hard gelatin capsules. Most commonly used pH-dependent coating polymers are methacrylic acid copolymers, commonly known as Eudragit® S, more specifically Eudragit® L and Eudragit® S. Eudragit® L100 and S 100 are copolymers of methacrylic acid and methyl methacrylate. Additional pH-dependent coating polymers include cellulose acetate phthalate (CAP), hydroxypropyl methylcellulose phthalate (HPMCP), polyvinyl acetate phthalate (PVAP) and cellulose acetate trimelliate.
(iii) Coating with biodegradable polymers;
(iv) Embeddingin matrices;
(v) Embedding in biodegradable matrices and hydrogels;
(vi) Embedding in pH-sensitive matrices;
(vii) Timed release systems;
(viii) Redox-sensitive polymers;
(ix) Bioadhesive systems;
(x) Coating with microparticles;
(xi) Osmotic controlled drug delivery.

Another approach towards colon-targeted drug delivery or controlled-release systems includes embedding the drug in polymer matrices to trap it and release it in the colon. These matrices can be pH-sensitive or biodegradable. Matrix-Based Systems, such as multi-matrix (MMX)-based delayed-release tablets, ensure the drug release in the colon.

Additional pharmaceutical approaches to targeted delivery of therapeutics to particular regions of the gastrointestinal tract are known. Chourasia M K, Jain S K, Pharmaceutical approaches to colon targeted drug delivery systems., J Pharm Sci. 2003 January-April; 6(1):33-66. Patel M, Shah T, Amin A. Therapeutic opportunities in colon-specific drug-delivery systems Crit Rev Ther Drug Carrier Syst. 2007; 24(2):147-202. KumarP, Mishra B. Colon targeted drug delivery systems—an overview. Curr Drug Deliv. 2008 July; 5(3):186-98. Van den Mooter G. Colon drug delivery. Expert Opin Drug Deliv. 2006 January; 3(1):111-25. Seth Amidon, Jack E. Brown, and Vivek S. Dave, Colon-Targeted Oral Drug Delivery Systems: Design Trends and Approaches, AAPS PharmSciTech. 2015 August; 16(4): 731-741.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of an HSD17B13 inhibitor. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In some embodiments, described herein is a method of treating or preventing a liver disease or condition in a mammal, comprising administering to the mammal a compound of Formula (I), (Ia), (Ib), (II), (IIa), or (IIb), or a pharmaceutically acceptable salt or solvate thereof. In some embodiments, the liver disease or condition is an alcoholic liver disease or condition. In some embodiments, the liver disease or condition is a nonalcoholic liver disease or condition. In some embodiments, the liver disease or condition is liver inflammation, fatty liver (steatosis), liver fibrosis, hepatitis, cirrhosis, hepatocellular carcinoma, or combinations thereof. In some embodiments, the liver disease or condition is primary biliary cirrhosis, primary sclerosing cholangitis, cholestasis, nonalcoholic steatohepatitis (NASH), nonalcoholic fatty liver disease (NAFLD), or combinations thereof. In some embodiments, the liver disease or condition described herein is a chronic liver disease or condition.

In some embodiments, described herein is a method of modulating HSD17B13 activity in a mammal, comprising administering to the mammal a compound of Formula (I), (Ia), (Ib), (II), (IIa), or (IIb), or a pharmaceutically acceptable salt or solvate thereof. In some embodiments, modulating comprises inhibiting HSD17B13 activity. In some embodiments of a method of modulating HSD17B13 activity in a mammal, the mammal has a liver disease or condition selected from liver inflammation, fatty liver (steatosis), liver fibrosis, hepatitis, cirrhosis, hepatocellular carcinoma, and combinations thereof. In some embodiments of a method of modulating HSD17B13 activity in a mammal, the mammal has a liver disease or condition selected from primary biliary cirrhosis, primary sclerosing cholangitis, cholestasis, nonalcoholic steatohepatitis (NASH), nonalcoholic fatty liver disease (NAFLD), and combinations thereof.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder, or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion, the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder, or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

It is understood that the dosage regimen to treat, prevent, or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

The compounds described herein, or a pharmaceutically acceptable salt thereof, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:
acac acetylacetone
ACN or MeCN acetonitrile
AcOH acetic acid
Ac acetyl
BBBPY 4,4′-di-tert-butyl-2,2′-dipyridyl
BINAP 2,2′-bis(diphenylphosphino)-1,1′-binaphthalene
Bn benzyl
BOC orfBoc tert-butyl carbamate
i-Bu iso-butyl
t-Bu tert-butyl
Cy cyclohexyl
CDI 1,1-carbonyldiimidazole
DBA or dba dibenzylideneacetone
DCE dichloroethane (ClCH₂CH₂Cl)
DCM dichloromethane (CH₂Cl₂)
DIBAL-H diisobutylaluminum hydride
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMPU N,N′-dimethylpropyleneurea
DMSO dimethylsulfoxide
DPPA diphenyl phosphoryl azide
Dppf or dppf 1,1′-bis(diphenylphosphino)ferrocene
EDC orEDCI N-(3-dimethylaminopropyl)-N′-ethylcarbodiimide hydrochloride
EEDQ 2-Ethoxy-1-ethoxycarbonyl-1,2-dihydroquinoline
eq equivalent(s)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxid hexafluorophosphate
HMPA hexamethylphosphoramide
HOBt 1-hydroxybenzotriazole
HPLC high performance liquid chromatography
IBX 2-iodoxybenzoic acid
KOAc potassium acetate
KOtBu potassium tert-butoxide
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
MCPBA or m-CPBA 3-chloroperbenzoic acid
2-MeTHF 2-methyltetrahydrofuran
Me methyl
MeOH methanol
MOM methoxymethyl ether
MS mass spectroscopy
Ms mesyl
MTBE methyl tert-butyl ether
NBS N-bromosuccinimide
NMM N-methyl-morpholine
NMP N-methyl-pyrrolidin-2-one
NMR nuclear magnetic resonance
OTf trifluoromethanesulfonate
PCC pyridinium chlorochromate
PE petroleum ether
Ph phenyl
PPTS pyridium p-toluenesulfonate
iPr/i-Pr iso-propyl
RP-HPLC reverse-phase high-pressure liquid chromatography
rt room temperature
SEM 2-(trimethylsilyl)ethoxymethyl
TBS tert-butyldimethylsilyl
TBAF tetra-n-butylammonium fluoride
TBAI tetra-n-butylammonium iodide
TEA triethylamine
TFA trifluoroacetic acid
THF tetrahy drofuran
TLC thin layer chromatography
TMEDA N,N,N,N-tetramethylethylenediamine
TMS trimethylsilyl
TsOH/p-TsOH p-toluenesulfonic acid

### Intermediate 1

### 2,3,5-Trifluoro-4-methoxybenzaldehyde

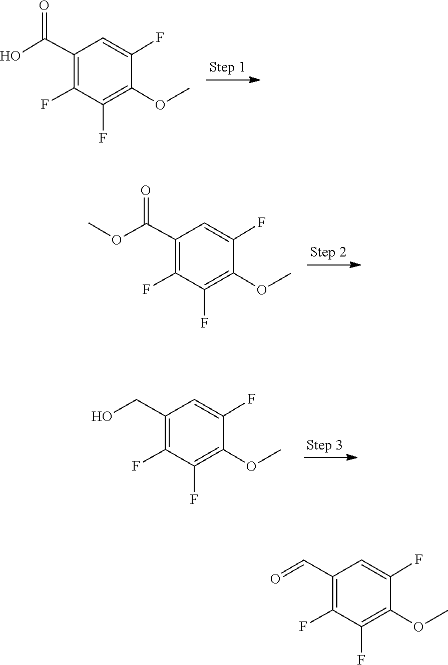

### Step 1: Methyl-2,3,5-trifluoro-4-methoxybenzoate

Iodomethane (1.2 mL, 19.4 mmol) was added to a mixture of 2,3,5-trifluoro-4-methoxy-benzoic acid (2 g, 9.70 mmol), K₂CO₃ (4.02 g, 29.1 mmol), and DMF (20 mL) at 0° C. The reaction mixture was stirred at 60° C. overnight, allowed to cool to rt, and then poured into H₂O (100 mL). The mixture was extracted with ethyl acetate (3×100 mL). The combined organic layers were washed with brine (100 mL), dried over Na₂SO₄, filtered, concentrated, and then purified by silica gel chromatography (petroleum ether/ethyl acetate=98:2) to give methyl-2,3,5-trifluoro-4-methoxybenzoate (1.8 g, 84%) as a white solid. ¹HNMR (400 MHz, DMSO-d₆): δ7.64-7.58 (m, 1H), 4.11 (s, 3H), 3.86 (s, 3H).

### Step 2: (2,3,5-Trifluoro-4-methoxyphenyl)methanol

Lithium aluminum hydride (621 mg, 16.4 mmol) was added to a solution of methyl-2,3,5-trifluoro-4-methoxy-benzoate (1.8 g, 8.18 mmol) in THF (20 mL) at 0° C. The reaction mixture was stirred at rt for 2 h, quenched with saturated NaK tartrate (˜100 mL), and then extracted with ethyl acetate (3×100 mL). The combined organic layers were washed with brine (100 mL), dried over Na₂SO₄, filtered, and then concentrated to give (2,3,5-trifluoro-4-methoxyphenyl)methanol (1.5 g) as a colorless liquid. ¹H NMR (400 MHz, DMSO-d₆): δ 7.25-7.11 (m, 1H), 5.47-5.43 (m, 1H), 4.52 (d, 2H), 3.86 (s, 3H).

### Step 3: 2,3,5-Trifluoro-4-methoxybenzaldehyde

Pyridinium chlorochromate (3.37 g, 15.6 mmol) and silica gel* (6.33 g, 105 mmol) were added to a mixture of (2,3,5-trifluoro-4-methoxyphenyl)methanol (1.5 g, 7.81 mmol) in CH₂Cl₂ (20 mL) at rt. The reaction mixture was stirred for 3 h and then filtered through Celite. The filter cake was washed with CH₂Cl₂ (2×˜10 mL). The filtrate was concentrated and then purified by silica gel chromatography (petroleum ether/ethyl acetate=98:2) to give 2,3,5-trifluoro-4-methoxybenzaldehyde (1.2 g, 81%) as a colorless liquid. ¹H NMR (400 MHz, DMSO-d₆): δ 10.06 (s, 1H), 7.63-7.55 (m, 1H), 4.15 (s, 3H).

### Intermediate 2

### 2,3,5-Trifluoro-4-(methoxymethoxy)benzaldehyde

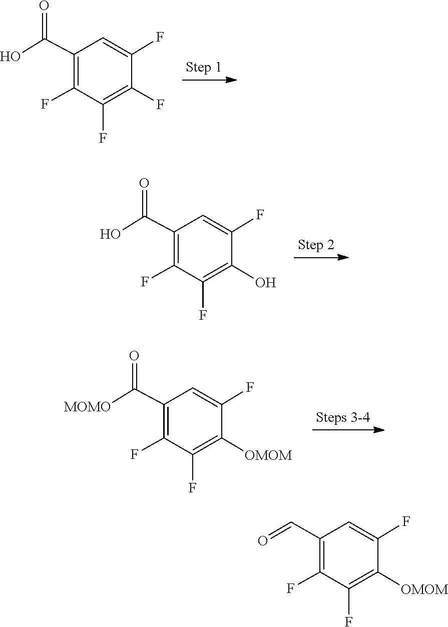

### Step 1: 2,3,5-Trifluoro-4-hydroxybenzoic acid

A mixture of 2,3,4,5-tetrafluorobenzoic acid (100 g, 515 mmol), NaOH (82.4 g, 2.06 mol), and H₂O (1400 mL) was stirred at 100° C. for 24 h and then cooled to 0° C. Aqueous hydrochloric acid solution (30%) was added dropwise until pH˜1. The solids were filtered and then dried under reduced pressure to give 2,3,5-trifluoro-4-hydroxybenzoic acid (72 g, 73%) as a white solid. ¹HNMR (400 MHz, DMSO-d₆): δ 13.9-11.2 (m, 2H), 7.41-7.52 (m, 1H).

### Step 2: Methoxymethyl 2,3,5-trifluoro-4-(methoxymethoxy)benzoate

A mixture of 2,3,5-trifluoro-4-hydroxybenzoic acid (80 g, 416 mmol), MOMCl (168 g, 2.09 mol), K₂CO₃ (345.4 g, 2.50 mol), and acetone (1100 mL) was stirred at rt for 48 h and then filtered. The filtrate was diluted with H₂O (1500 mL) and extracted with EtOAc (4×1000 mL). The combined organic layers were washed with brine (2000 mL), dried over Na₂SO₄, concentrated, and then purified by silica gel chromatography (petroleum ether/EtOAc=100:1 to 20:1) to give methoxymethyl 2,3,5-trifluoro-4-(methoxymethoxy)benzoate (60 g, 510%) as a yellow oil. ¹H NMR (400 MHz, DMSO-d₆): δ 7.51-7.53 (m, 1H), 5.47-5.49 (s, 2H), 5.30-5.29 (m, 2H), 3.59 (s, 3H), 3.56 (s, 3H).

### Step 3: (2,3,5-Trifluoro-4-(methoxymethoxy)phenyl)methanol

Lithium aluminum hydride (16.25 g, 428.3 mmol) was added to a mixture of methoxymethyl 2,3,5-trifluoro-4-(methoxymethoxy)benzoate (60 g, 214.2 mmol) in THF (600 mL) at 0° C. under N₂. The mixture was stirred at rt for 1 h and then quenched with saturated NaK tartrate (˜1000 mL) at 0° C. The mixture was extracted with EtOAc (4×600 mL). The combined organic layers were dried over Na₂SO₄, concentrated, and then purified by silica gel chromatography (petroleum ether/EtOAc=10:1 to 5:1) to give (2,3,5-trifluoro-4-(methoxymethoxy)phenyl)methanol (40 g, 84%) as a yellow oil. ¹H NMR (400 MHz, DMSO-d₆): δ 7.27-7.00 (m, 1H), 5.18 (s, 2H), 4.73 (s, 2H), 3.60 (s, 3H).

### Step 4: 2,3,5-Trifluoro-4-(methoxymethoxy)benzaldehyde

Pyridinium chlorochromate (116 g, 540 mmol) and silica gel (116 g, 1.94 mol) were added to a mixture of (2,3,5-trifluoro-4-(methoxymethoxy)phenyl)methanol (40 g, 180 mmol) in CH₂Cl₂ (400 mL) at rt. The mixture was stirred for 2 h and then filtered. The filtrate was concentrated and purified by silica gel chromatography (petroleum ether/EtOAc=100:1 to 2:1) to give 2,3,5-trifluoro-4-(methoxymethoxy)benzaldehyde (29.0 g, 72%) as a yellow oil. ¹H NMR (400 MHz, DMSO-d₆): δ 10.26-10.25 (s, 1H), 7.44-7.40 (m, 1H), 5.32 (s, 2H), 3.60 (s, 3H); LCMS: 221.0 [M+H]⁺.

### Intermediate 3

### 4-(Benzyloxy)-2,3,5-trifluorobenzaldehyde

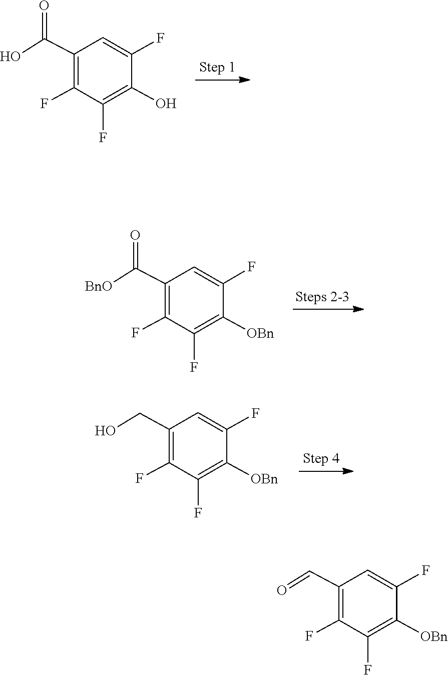

### Step 1: Benzyl 4-(benzyloxy)-2,3,5-trifluorobenzoate

Benzyl bromide (206 g, 1.21 mol) and K₂CO₃ (278 g, 2.01 mol) were added to a mixture of 2,3,5-trifluoro-4-hydroxy-benzoic acid (77.3 g, 402 mmol) in DMF (800 mL). The reaction mixture was stirred at rt overnight, poured into H₂O (500 mL), and then extracted with 5:1 petroleum ether/EtOAc (3×400 mL). The combined organic layers were washed with brine (400 mL), dried over Na₂SO₄, filtered, concentrated, and then purified by silica gel chromatography (petroleum ether/EtOAc=1/0 to 50/1) to give benzyl 4-benzyloxy-2,3,5-trifluoro-benzoate (131 g, 87%) as a white solid. ¹H NMR (400 MHz, DMSO-d₆): δ 7.71-7.55 (m, 1H), 7.47-7.29 (m, 10H), 5.35 (d, 4H).

### Step 2: 4-(Benzyloxy)-2,3,5-trifluorobenzoic acid

Sodium hydroxide (141 g, 3.52 mol) was added to a solution of benzyl 4-benzyloxy-2,3,5-trifluoro-benzoate (131 g, 352 mmol) in EtOH (1500 mL) and H₂O (750 mL) at rt. The mixture was stirred at 80° C. overnight, cooled to rt, concentrated to remove EtOH, diluted with water (600 mL), and then adjusted to pH˜3 with aqueous HCl (˜30%). The precipitate was filtered, and the filter cake was dried under high vacuum and then triturated (450 mL of petroleum ether/EtOAc=20/1) to give 4-benzyloxy-2,3,5-trifluoro-benzoic acid (90 g, 90%) as a yellow solid. ¹H NMR (400 MHz, DMSO-d₆): δ 13.67 (s, 1H), 7.50-7.25 (m, 5H), 7.68-7.53 (m, 1H), 5.35 (s, 2H); LCMS: 281.0 [M−H]⁻.

### Step 3: (4-(Benzyloxy)-2,3,5-trifluorophenyl)methanol

Borane dimethyl sulfide complex solution (10 M in Me₂S, 95.67 mL) was added dropwise to a solution of 4-benzyloxy-2,3,5-trifluoro-benzoic acid (90 g, 319 mmol) in THF (900 mL) at 0-5° C. under N₂. The mixture was stirred at 80° C. for 2 h, cooled to 0° C., and then quenched with CH₃OH (200 mL) at 0-5° C. The mixture was stirred at 70° C. for 1 h, cooled to rt, concentrated, and then purified by silica gel chromatography (petroleum ether/EtOAc=50/1 to 10/1) to give (4-benzyloxy-2,3,5-trifluoro-phenyl)methanol (82 g, 95%) as a yellow solid. ¹HNMR (400 MHz, DMSO-d₆): δ 7.55-7.28 (m, 5H), 7.21-7.08 (m, 1H), 5.53-5.40 (m, 1H), 5.20 (s, 2H), 4.58-4.40 (m, 2H).

### Step 4: 4-(Benzyloxy)-2,3,5-trifluorobenzaldehyde

Silica gel (198 g, 3.29 mol) and pyridinium chlorochromate (198 g, 917 mmol) were added to a mixture of (4-benzyloxy-2,3,5-trifluoro-phenyl)methanol (82 g, 306 mmol) in CH₂Cl₂ (820 mL). The mixture was stirred at rt for 2 h, filtered, concentrated, and then purified by silica gel chromatography (petroleum ether/EtOAc=1/0 to 50/1) to give 4-benzyloxy-2,3,5-trifluorobenzaldehyde (70 g, 86%) as a white solid. ¹HNMR (400 MHz, DMSO-d₆): δ 10.05 (d, 1H), 7.70-7.52 (m, 1H), 7.49-7.31 (m, 5H), 5.41 (s, 2H); LCMS: 267.0 [M+H]⁺.

### Intermediate 4

### 4-(Benzyloxy)-3,5-difluoro-2-iodobenzaldehyde

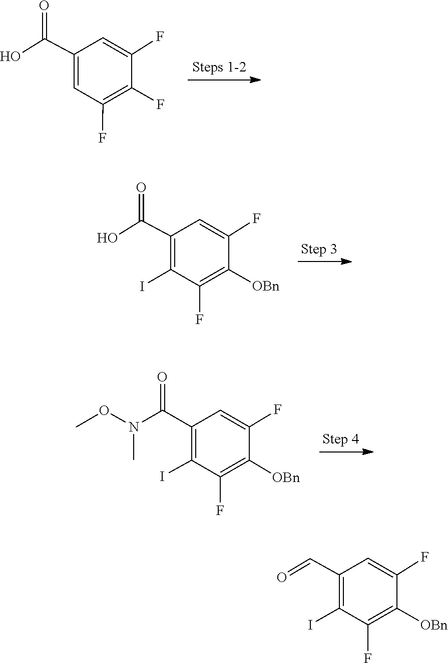 [SMILES: O=C(O)c1cc(F)c(F)c(F)c1 ; O=C(O)c1cc(F)c(OCc2ccccc2)c(F)c1I ; CON(C)C(=O)c1cc(F)c(OCc2ccccc2)c(F)c1I ; O=Cc1cc(F)c(OCc2ccccc2)c(F)c1I]

### Step 1: 4-(Benzyloxy)-3,5-difluorobenzoic acid

Sodium hydride (60%, 17.03 g, 425.91 mmol) was added to a mixture of 3,4,5-trifluorobenzoic acid (25 g, 142 mmol), phenylmethanol (15.35 g, 142 mmol), and DMF (500 mL) at 0° C. under N₂. The mixture was stirred at rt overnight and then poured into water (400 mL) slowly. The mixture was combined with five other crude batches of the same scale and adjusted to pH˜3 with concentrated HCl (˜200 mL). The mixture was filtered. The filter cake was washed with water (500 mL) and then dried under high vacuum to give 4-(benzyloxy)-3,5-difluorobenzoic acid (165 g) as a light yellow solid. ¹H NMR (400 MHz, DMSO-d₆): δ 13.41 (s, 1H), 7.64-7.54 (m, 2H), 7.46-7.31 (m, 5H), 5.29 (s, 2H); LCMS: 263.0 [M−H]⁻.

### Step 2: 4-(Benzyloxy)-3,5-difluoro-2-iodobenzoic acid

n-Butyllithium solution (2.5 M in n-hexane, 255 mL, 638 mmol) was added dropwise to a solution of 4-(benzyloxy)-3,5-difluorobenzoic acid (67 g, 254 mmol) in THF (1 L) at −78° C. under N₂. The mixture was stirred at −78° C. for 2 h. A solution of I₂ (160.90 g, 633.93 mmol) in THF (1 L) was added at −78° C. The mixture was warmed to rt, stirred overnight, and then quenched with saturated Na₂S₂O₃ (1 L). The mixture was combined with three other crude batches of the same scale and extracted with EtOAc (3×2 L). The combined organic layers were washed with brine (2 L), dried over Na₂SO₄, filtered, and then concentrated to give 4-(benzyloxy)-3,5-difluoro-2-iodobenzoic acid (350 g) as a yellow solid. ¹H NMR (400 MHz, DMSO-d₆): δ 7.54-7.09 (m, 6H), 5.12 (s, 2H); LCMS: 388.8 [M−H]⁻.

### Step 3: 4-(Benzyloxy)-3,5-difluoro-2-iodo-N-methoxy-N-methylbenzamide

N,O-Dimethylhydroxylamine (38.4 g, 394 mmol, HCl salt) and then T₃P (522 g, 820 mmol, 50% purity in EtOAc) were added to a mixture of 4-(benzyloxy)-3,5-difluoro-2-iodobenzoic acid (128 g, 295 mmol) and Et₃N (133 g, 1.31 mol) in CH₂Cl₂ (1.5 L). The mixture was stirred at rt overnight and then poured into water (2 L). The mixture was combined with three other crude batches of the same scale and extracted with CH₂Cl₂ (3×2 L). The combined organic layers were washed with brine (2 L), dried over Na₂SO₄, filtered, concentrated, and then purified by silica gel chromatography (petroleum ether/EtOAc=8/1) to give 4-benzyloxy-3,5-difluoro-2-iodo-N-methoxy-N-methyl-benzamide (166 g, 40% over 3 steps) as a yellow solid. ¹HNMR (400 MHz, DMSO-d₆): δ 7.47-7.27 (m, 6H), 5.21 (s, 2H), 3.44 (s, 3H), 3.27 (s, 3H); LCMS: 433.9 [M+H]⁺.

### Step 4: 4-(Benzyloxy)-3,5-difluoro-2-iodobenzaldehyde

Diisobutylaluminum hydride (1 M in toluene, 290 mL, 290 mmol) was added dropwise to a solution of 4-benzyloxy-3,5-difluoro-2-iodo-N-methoxy-N-methyl-benzamide (83 g, 192 mmol) in CH₂Cl₂ (1 L) at −78° C. under N₂. The reaction mixture was stirred at −78° C. for 2 h and then quenched with saturated NaK tartrate (1.2 L) keeping the temperature less than 5° C. The mixture was stirred at rt overnight and then combined with another crude batch of the same scale. This mixture was filtered through Celite. The filtrate was extracted with CH₂Cl₂ (3×1 L). The combined organic layers were washed with brine (1 L), dried over Na₂SO₄, filtered, concentrated, and then purified by silica gel chromatography (petroleum ether/ethyl acetate=95/5) to give 4-(benzyloxy)-3,5-difluoro-2-iodobenzaldehyde (130 g, 90%) as a white solid. ¹H NMR (400 MHz, DMSO-d₆): δ 9.87 (d, 1H), 7.60 (dd, 1H), 7.50-7.28 (m, 5H), 5.35 (s, 2H).

The Intermediate below was synthesized from 5-fluoro-6-methoxynicotinic acid following the procedures described for Intermediate 4, Steps 2-4.

| Int | Structure | Name | [M + H]⁺ |
|---|---|---|---|
| 4.1 | 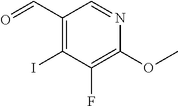 | 5-Fluoro-4-iodo-6- methoxynicotinaldehyde | 282.0 |

### Intermediate 5

### 6-(Benzyloxy)-5,7-difluoro-1H-indazole

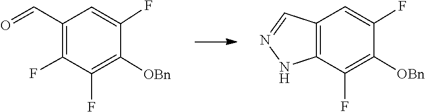 [SMILES: O=Cc1cc(F)c(OCc2ccccc2)c(F)c1F ; Fc1cc2cn[nH]c2c(F)c1OCc1ccccc1]

Hydrazine hydrate (79 mL, 1.60 mol, 98%) was added slowly to a solution of Intermediate 3 (20 g, 75.13 mmol) in DME (200 mL) at rt. The mixture was stirred at 105° C. for 16 h, cooled to rt, diluted with water (100 mL), and then extracted with EtOAc (3×100 mL). The combined organic layers were washed with brine (2×100 mL), dried over Na₂SO₄, filtered, concentrated, and then purified by silica gel chromatography (petroleum ether/EtOAc=100/1 to 20/1) to give 6-benzyloxy-5,7-difluoro-1H-indazole (2.1 g, 10%) as a yellow solid. ¹HNMR (400 MHz, DMSO-d₆): δ 13.63 (s, 1H), 8.11 (s, 1H), 7.45-7.17 (m, 6H), 5.22 (s, 2H); LCMS: 261.0 [M+H]⁺.

The Intermediate below was synthesized from Intermediate 39 following the procedure described for Intermediate 5.

| Int | Structure | Name | [M + H]⁺ |
|---|---|---|---|
| 5.1 | 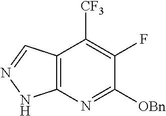 [SMILES: Fc1c(OCc2ccccc2)nc2[nH]ncc2c1C(F)(F)F] | 6-(Benzyloxy)-5-fluoro-4- (trifluoromethyl)-1H-pyrazolo[3,4- b]pyridine | 312.1 |

### Intermediate 5

### 6-(Benzyloxy)-5,7-difluoro-1H-indazole

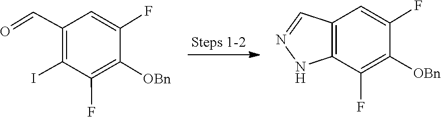 [SMILES: O=Cc1cc(F)c(OCc2ccccc2)c(F)c1I ; Fc1cc2cn[nH]c2c(F)c1OCc1ccccc1]

### Step 1: (E)-N′-(4-(Benzyloxy)-3,5-difluoro-2-iodobenzylidene)-4-methylbenzenesulfonohydrazide

Concentrated hydrochloric acid (5.2 mL, 53.8 mmol) was added to a mixture of Intermediate 4 (130 g, 347 mmol), 4-methylbenzenesulfonohydrazide (71.2 g, 382 mmol), and EtOH (1300 mL). The mixture was warmed to 50° C., stirred overnight, allowed to cool to rt, and then filtered. The filter cake was washed with ice cold EtOH (200 mL) and then dried under high vacuum to give (E)-N′-(4-(benzyloxy)-3,5-difluoro-2-iodobenzylidene)-4-methylbenzenesulfonohydrazide (160 g, 85%) as a white solid. ¹H NMR (400 MHz, DMSO-d₆): δ 11.85 (s, 1H), 8.09 (d, 1H), 7.77 (d, 2H), 7.48-7.27 (m, 8H), 5.20 (s, 2H), 2.36 (s, 3H); LCMS: 543.0 [M+H]⁺.

### Step 2: 6-(Benzyloxy)-5,7-difluoro-1H-indazole

A mixture of (E)-N′-(4-(benzyloxy)-3,5-difluoro-2-iodobenzylidene)-4-methylbenzenesulfonohydrazide (65 g, 120 mmol), 3-methylbutan-1-ol (1.5 L), and Cu₂O (8.57 g, 59.9 mmol) was refluxed overnight under N₂, cooled to rt, and then poured into water (1.5 L). The mixture was combined with another crude batch of the same scale and extracted with EtOAc (3×2 L). The combined organic layers were washed with brine (2 L), dried over Na₂SO₄, filtered, concentrated, and then purified by silica gel chromatography (petroleum ether/ethyl acetate=80/20). The material was triturated in petroleum ether/ethyl acetate (10/1, 99 mL), stirred at rt overnight, and then filtered. The filter cake was washed with ice cold petroleum ether/ethyl acetate (20/1, 20 mL) and then dried under high vacuum to give 6-(benzyloxy)-5,7-difluoro-1H-indazole (30 g, 48%) as a light yellow solid. ¹H NMR (400 MHz, DMSO-d₆): δ 13.64 (s, 1H), 8.10 (d, 1H), 7.53-7.42 (m, 3H), 7.41-7.30 (m, 3H), 5.22 (s, 2H); LCMS: 261.0 [M+H]⁺.

### Intermediate 6

### 6-(Benzyloxy)-3,5,7-trifluoro-1H-indazole

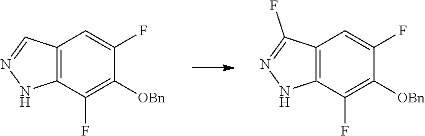 [SMILES: Fc1cc2cn[nH]c2c(F)c1OCc1ccccc1 ; Fc1cc2c(F)n[nH]c2c(F)c1OCc1ccccc1]

1-Chloromethyl-4-fluoro-1,4-diazoniabicyclo[2.2.2]octane bis(tetrafluoroborate) (1.50 g, 4.23 mmol) was added to a solution of Intermediate 5 (1.00 g, 3.84 mmol) in CH₃CN (10 mL) under N₂. The mixture was refluxed for 12 h, cooled to rt, filtered, concentrated, and then purified by column chromatography (petroleum ether/ethyl acetate-100/1 to 1/1) to give 6-(benzyloxy)-3,5,7-trifluoro-1H-indazole (180 mg, 17%) as a yellow solid. ¹H NMR (400 MHz, DMSO-d₆): δ 13.21 (s, 1H), 7.55 (d, 1H), 7.32-7.48 (m, 5H), 5.25 (s, 2H); LCMS: 279.1 [M+H]⁺.

### Intermediate 7

### 3,5-Difluoro-1H-indazol-6-ol

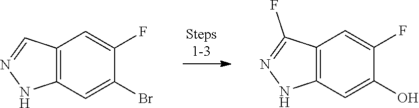 [SMILES: Fc1cc2cn[nH]c2cc1Br ; Oc1cc2[nH]nc(F)c2cc1F]

### Step 1: 6-Bromo-3,5-difluoro-1H-indazole

1-(Chloromethyl)-4-fluoro-1,4-diazoniabicyclo[2.2.2]octane bis(tetrafluoroborate) (4.35 g, 12.3 mmol) was added to a solution of 6-bromo-5-fluoro-1H-indazole (2.40 g, 11.2 mmol) in CH₃CN (30 mL). The mixture was stirred at 90° C. for 4 h, allowed to cool to rt, concentrated, and then purified by column chromatography (petroleum ether/ethyl acetate=20/1 to 5/1). The material was purified further by reverse-phase HPLC [water (0.04% HCl)—CH₃CN] to give 6-bromo-3,5-difluoro-1H-indazole (740 mg, 28%) as a white solid. ¹H NMR (400 MHz, CDCl₃): δ 9.21 (s, 1H), 7.66 (d, 1H), 7.41 (d, 1H); LCMS: 233.0 [M+H]⁺.

### Step 2: 3,5-Difluoro-6-(4,4,5,5-tetramethyl-1,3,2-dioxaborolan-2-yl)-1H-indazole

Potassium acetate (623 mg, 6.35 mmol) and Pd(dppf)Cl₂ (116 mg, 0.158 mmol) were added to a solution of 6-bromo-3,5-difluoro-1H-indazole (740 mg, 3.18 mmol), 4,4,5,5-tetramethyl-2-(4,4,5,5-tetramethyl-1,3,2-dioxaborolan-2-yl)-1,3,2-dioxaborolane (968 mg, 3.81 mmol), and dioxane (8 mL) under N₂. The mixture was degassed with 3 vacuum/N₂ cycles, stirred at 105° C. overnight, allowed to cool to rt, and then filtered through Celite. The filtrate was poured into water (10 mL) and extracted with ethyl acetate (2×20 mL). The combined organic layers were washed with brine (30 mL), dried over Na₂SO₄, filtered, concentrated, and then purified by column chromatography (petroleum ether/ethyl acetate=20/1 to 5/1) to give 3,5-difluoro-6-(4,4,5,5-tetramethyl-1,3,2-dioxaborolan-2-yl)-1H-indazole (600 mg, 67%) as a white solid. ¹H NMR (400 MHz, DMSO-d₆): δ 12.74 (s, 1H), 7.74 (d, 1H), 7.47 (d, 1H), 1.32 (s, 12H); LCMS: 281.2 [M+H]⁺.

### Step 3: 3,5-Difluoro-1H-indazol-6-ol

Hydrogen peroxide solution (1 mL, 10.7 mmol, 30% in water) was added to a solution of 3,5-difluoro-6-(4,4,5,5-tetramethyl-1,3,2-dioxaborolan-2-yl)-1H-indazole (600 mg, 2.14 mmol), aqueous NaOH (1 M, 19.5 mL), and THF (14 mL) at 0° C. The mixture was stirred at 0° C. for 1 h, poured into H₂O (30 mL), and then extracted with ethyl acetate (3×30 mL). The combined organic layers were washed with aqueous Na₂SO₃ (2×20 mL), washed with brine (2×20 mL), dried over Na₂SO₄, filtered, concentrated, and then purified by column chromatography (petroleum ether/ethyl acetate=10/1 to 1/1) to give 3,5-difluoro-1H-indazol-6-ol (330 mg, 91%) as a white solid. ¹HNMR (400 MHz, DMSO-d₆): δ 12.12 (s, 1H), 10.41 (s, 1H), 7.44 (d, 1H), 6.87 (dd, 1H); LCMS: 171.1 [M+H]⁺.

### Intermediate 8

### 4,7-Difluoro-1H-indazol-6-ol

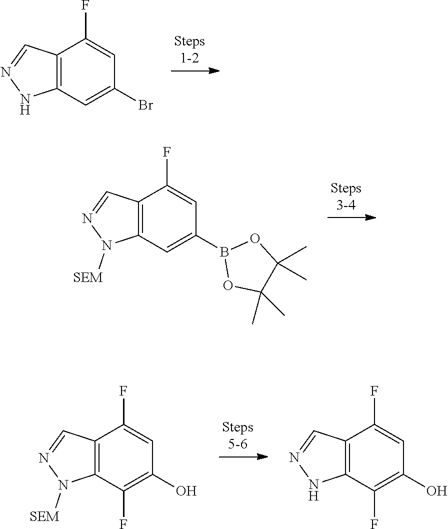 [SMILES: Fc1cc(Br)cc2[nH]ncc12 ; CC1(C)OB(c2cc(F)c3cnn(COCC[Si](C)(C)C)c3c2)OC1(C)C ; C[Si](C)(C)CCOCn1ncc2c(F)cc(O)c(F)c21 ; Oc1cc(F)c2cn[nH]c2c1F]

### Step 1: 6-Bromo-4-fluoro-1-((2-(trimethylsilyl)ethoxy)methyl)-1H-indazole

Sodium hydride (60%, 2.79 g, 69.8 mmol) was added carefully to a solution of 6-bromo-4-fluoro-1H-indazole (10 g, 46.5 mmol) in DMF (200 mL) at 0° C. The mixture was stirred at 0° C. for 1 h. SEM-Cl (15.51 g, 93.01 mmol) was added at 0° C. slowly. The mixture was allowed to warm to rt overnight, poured into water (300 mL), and then extracted with EtOAc (2×300 mL). The combined organic layers were washed with water (2×200 mL), washed with brine (200 mL), dried (Na₂SO₄), filtered, concentrated, and then purified by silica gel chromatography (petroleum ether/EtOAc=15/1) to give 6-bromo-4-fluoro-1-((2-(trimethylsilyl)ethoxy)methyl)-1H-indazole (15.1 g, 94%) as a yellow solid. ¹H NMR (400 MHz, DMSO-d₆): δ 8.42 (s, 1H), 8.09 (s, 1H), 7.38 (d, 1H), 5.90 (s, 2H), 3.73-3.55 (m, 2H), 1.05-0.83 (m, 2H) 0.00 (s, 9H); LCMS: 345.0 [M+H]⁺.

### Step 2: 4-Fluoro-6-(4,4,5,5-tetramethyl-1,3,2-dioxaborolan-2-yl)-1-((2-(trimethylsilyl)ethoxy)methyl)-1H-indazole

A mixture of 6-bromo-4-fluoro-1-((2-(trimethylsilyl)ethoxy)methyl)-1H-indazole (15.1 g, 43.7 mmol), 4,4,4′,4′,5,5,5′,5′-octamethyl-2,2′-bi(1,3,2-dioxaborolane) (13.9 g, 54.9 mmol), Pd(dppf)Cl₂ (3.20 g, 4.37 mmol), KOAc (21.5 g, 219 mmol), and dioxane (300 mL) was stirred at 90° C. overnight under N₂. The mixture was allowed to cooled to rt, poured into water (300 mL), and then extracted with EtOAc (2×300 mL). The combined organic layers were washed with water (2×200 mL), washed with brine (200 mL), dried (Na₂SO₄), filtered, concentrated, and then purified by silica gel chromatography (petroleum ether/EtOAc=20/1) to give 4-fluoro-6-(4,4,5,5-tetramethyl-1,3,2-dioxaborolan-2-yl)-1-((2-(trimethylsilyl)ethoxy)methyl)-1H-indazole (17 g) as a yellow oil. ¹H NMR (400 MHz, DMSO-d₆): δ 8.42 (d, 1H), 8.02 (s, 1H), 7.20 (d, 1H), 5.97 (s, 2H), 3.72-3.52 (m, 2H), 1.05 (s, 12H), 1.05-0.83 (m, 2H), 0.00 (d, 9H); LCMS: 393.2 [M+H]⁺.

### Step 3: 4-Fluoro-1-((2-(trimethylsilyl)ethoxy)methyl)-1H-indazol-6-ol

A mixture of 4-fluoro-6-(4,4,5,5-tetramethyl-1,3,2-dioxaborolan-2-yl)-1-((2-(trimethylsilyl)ethoxy)methyl)-1H-indazole (17 g, 43 mmol), NaBO₃·4H₂O (26.7 g, 173 mmol), THF (200 mL), and CH₃OH (100 mL) was stirred at rt overnight, poured into water (200 mL), and then extracted with EtOAc (2×200 mL). The combined organic layers were washed with water (2×200 mL), washed with brine (200 mL), dried (Na₂SO₄), filtered, concentrated, and then purified by silica gel chromatography (petroleum ether/EtOAc=20/1) to give 4-fluoro-1-((2-(trimethylsilyl)ethoxy)methyl)-1H-indazol-6-ol (8 g, 65%) as a yellow solid. ¹HNMR (400 MHz, DMSO-d₆): δ 10.23 (s, 1H), 8.13 (s, 1H), 6.88 (s, 1H), 6.60 (d, 1H), 5.72 (s, 2H), 3.60 (t, 2H), 0.90 (t, 2H), 0.01 (s, 9H); LCMS: 283.1 [M+H]⁺.

### Step 4: 4,7-Difluoro-1-((2-(trimethylsilyl)ethoxy)methyl)-1H-indazol-6-ol

A mixture of 4-fluoro-1-((2-(trimethylsilyl)ethoxy)methyl)-1H-indazol-6-ol (4 g, 14.2 mmol), 1-fluoropyridin-1-ium trifluoromethanesulfonate (3.85 g, 15.6 mmol), and DCE (150 mL) was stirred at 80° C. for 5 h. The mixture was allowed to cool to rt, concentrated to dryness, and then purified by silica gel chromatography (petroleum ether/EtOAc=20/1) to give 4,7-difluoro-1-((2-(trimethylsilyl)ethoxy)methyl)-1H-indazol-6-ol (0.9 g, 21%) as a yellow solid. ¹HNMR (400 MHz, DMSO-d₆): δ 10.59 (s, 1H), 8.26 (d, 1H), 6.78 (d, 1H), 5.80 (s, 2H), 3.72-3.60 (m, 2H), 0.95-0.86 (m, 2H), 0.05 (s, 9H); LCMS: 299.0 [M−H]⁻.

### Step 5: 4,7-Difluoro-1-(hydroxymethyl)-1H-indazol-6-ol

A mixture of 4,7-difluoro-1-((2-(trimethylsilyl)ethoxy)methyl)-1H-indazol-6-ol (1 g, 3.33 mmol), TFA (6.16 g, 54.0 mmol), and CH₂Cl₂ (10 mL) was stirred at rt overnight. The mixture was poured into NaHCO₃ (20 mL) and extracted with EtOAc (2×20 mL). The combined organic layers were washed with water (2×10 mL), washed with brine (10 mL), dried (Na₂SO₄), filtered, and then concentrated to give. 4,7-difluoro-1-(hydroxymethyl)-1H-indazol-6-ol (666 mg) as a yellow oil. LCMS: 201.1 [M+H]⁺.

### Step 6: 4,7-Difluoro-1H-indazol-6-ol

A mixture of 4,7-difluoro-1-(hydroxymethyl)-1H-indazol-6-ol (666 mg, 3.33 mmol), ethane-1,2-diamine (6.29 g, 105 mmol), and EtOH (10 mL) was stirred at rt for 4 h. The mixture was concentrated, adjusted to pH=3 with 1 M HCl, and then extracted with EtOAc (2×20 mL). The combined organic layers were washed with water (2×10 mL), washed with brine (10 mL), dried (Na₂SO₄), filtered, concentrated, and then purified by silica gel chromatography (petroleum ether/EtOAc=8/1) to give 4,7-difluoro-1H-indazol-6-ol (200 mg, 35%) as a yellow solid. ¹HNMR (400 MHz, DMSO-d₆): δ 13.56 (s, 1H), 10.33 (s, 1H), 8.12 (s, 1H), 6.63 (d, 1H); LCMS: 171.1 [M+H]⁺.

### Intermediate 9

### 5-Fluoro-7-methyl-1H-imidazol-6-ol

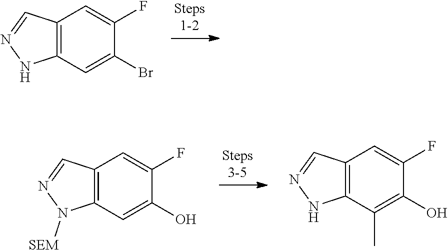 [SMILES: Fc1cc2cn[nH]c2cc1Br ; C[Si](C)(C)CCOCn1ncc2cc(F)c(O)cc21 ; Cc1c(O)c(F)cc2cn[nH]c12]

### Step 1: 6-Bromo-5-fluoro-1-((2-(trimethylsilyl)ethoxy)methyl)-1H-indazole

Sodium hydride (60%, 586 mg, 14.7 mmol) was added in three portions to a solution of 6-bromo-5-fluoro-1H-indazole (2.10 g, 9.77 mmol) in DMF (20 mL) at 0° C. under N₂. The mixture was stirred at 0° C. for 1 h. 2-(Trimethylsilyl)ethoxymethyl chloride (3.46 mL, 19.5 mmol) was added at 0° C. The reaction was allowed to warm to rt, stirred overnight, poured into H₂O (100 mL), and then extracted with EtOAc (3×100 mL). The combined organic layers were washed with brine (2×100 mL), dried over Na₂SO₄, filtered, concentrated, and then purified by column chromatography (petroleum ether/ethyl acetate=50/1 to 20/1) to give 6-bromo-5-fluoro-1-((2-(trimethylsilyl)ethoxy)methyl)-1H-indazole (3.30 g) as a yellow oil. ¹H NMR (400 MHz, DMSO-d₆): δ 8.24 (d, 1H), 8.15 (s, 1H), 7.78 (d, 1H), 5.75 (s, 2H), 3.49 (t, 2H), 0.77 (t, 2H), −0.12 (s, 9H); LCMS: 345.1 [M+H]⁺.

### Step 2: 5-Fluoro-1-((2-(trimethylsilyl)ethoxy)methyl)-1H-indazol-6-ol

Pd₂(dba)₃ (875 mg, 0.955 mmol) was added to a mixture of 6-bromo-5-fluoro-1-((2-(trimethylsilyl)ethoxy)methyl)-1H-indazole (3.30 g, 9.56 mmol), Cs₂CO₃ (12.5 g, 38.2 mmol), t-BuXPhos (812 mg, 1.91 mmol), dioxane (60 mL), and H₂O (12 mL). The mixture was degassed with 3 vacuum/N₂ cycles, stirred at 90° C. for 2 h, allowed to cool to rt, and then filtered through Celite. The filtrate was poured into water (40 mL) and extracted with EtOAc (3×50 mL). The combined organic layers were washed with brine (2×50 mL), dried over Na₂SO₄, filtered, concentrated, and then purified by column chromatography (petroleum ether/ethyl acetate=20/1 to 5/1) to give 5-fluoro-1-((2-(trimethylsilyl)ethoxy)methyl)-1H-indazol-6-ol (1.60 g, 59%) as a yellow solid. ¹H NMR (400 MHz, DMSO-d₆): δ 10.27 (s, 1H), 7.93 (s, 1H), 7.50 (d, 1H), 7.10 (d, 1H), 5.61 (s, 2H), 3.47 (t, 2H), 0.78 (t, 2H), −0.10 (s, 9H); LCMS: 283.1 [M+H]⁺.

### Step 3: 5-Fluoro-7-(hydroxymethyl)-1-((2-(trimethylsilyl)ethoxy)methyl)-1H-indazol-6-ol

Aqueous potassium hydroxide (0.6M, 3.5 mL, 2.1 mmol) and then formaldehyde (37% in water, 1 mL, 13.3 mmol) were added to a solution of 5-fluoro-1-(2-trimethylsilylethoxymethyl)indazol-6-ol (1.20 g, 4.25 mmol) in THF (12 mL). The mixture was heated at 55° C. for 2 h, cooled to rt, stirred overnight, diluted with saturated NH₄Cl (30 mL), and then extracted with EtOAc (3×50 mL). The combined organic layers were washed with brine (30 mL), dried over Na₂SO₄, filtered, concentrated, and then purified by column chromatography (petroleum ether/ethyl acetate=20/1 to 5/1) to give 5-fluoro-7-(hydroxymethyl)-1-((2-(trimethylsilyl)ethoxy)methyl)-1H-indazol-6-ol (600 mg, 45%) as a gray solid. ¹H NMR (400 MHz, DMSO-d₆): δ 9.80 (s, 1H), 7.95 (s, 1H), 7.49 (s, 1H), 5.82 (s, 2H), 5.21 (t, 1H), 4.86 (d, 2H), 3.47 (t, 2H), 0.76 (t, 2H), −0.13 (s, 9H); LCMS: 313.2 [M+H]⁺.

### Step 4: 5-Fluoro-7-methyl-1-((2-(trimethylsilyl)ethoxy)methyl)-1H-indazol-6-ol

Ammonium formate (1.01 g, 16.0 mmol) was added to a mixture of 5-fluoro-7-(hydroxymethyl)-1-((2-(trimethylsilyl)ethoxy)methyl)-1H-indazol-6-ol (500 mg, 1.60 mmol), 10% Pd/C (250 mg, 0.23 mmol), and EtOH (2 mL) at 80° C. The mixture was stirred at 80° C. overnight, allowed to cool to rt, filtered, concentrated, and then purified by column chromatography (petroleum ether/ethyl acetate=20/1 to 5/1) to give 5-fluoro-7-methyl-1-((2-(trimethylsilyl)ethoxy)methyl)-1H-indazol-6-ol (370 mg, 77%) as a white solid. ¹H NMR (400 MHz, DMSO-d₆): δ 9.54 (s, 1H), 7.91 (s, 1H), 7.35 (d, 1H), 5.74 (s, 2H), 3.45 (t, 2H), 2.53 (s, 3H), 0.78 (t, 2H), −0.12 (s, 9H); LCMS: 297.2 [M+H]⁺.

### Step 5: 5-Fluoro-7-methyl-1H-indazol-6-ol

Concentrated sulfuric acid (0.66 mL, 12.48 mmol) was added to a solution of 5-fluoro-7-methyl-1-((2-(trimethylsilyl)ethoxy)methyl)-1H-indazol-6-ol (370 mg, 1.25 mmol) in dioxane (5 mL). The mixture was stirred at 40° C. for 3 h and then concentrated. The residue was dispersed into H₂O (20 mL) and extracted with EtOAc (3 xl10 mL). The combined organic layers were washed with brine (2×10 mL), dried over Na₂SO₄, filtered, concentrated, and then purified by column chromatography (petroleum ether/ethyl acetate=10/1 to 1/1) to give 5-fluoro-7-methyl-1H-indazol-6-ol (160 mg, 77%) as a white solid. ¹HNMR (400 MHz, DMSO-d₆): δ 12.86 (s, 1H), 9.36 (s, 1H), 7.88 (s, 1H), 7.30 (s, 1H), 2.34 (s, 3H); LCMS: 167.1 [M+H]⁺.

### Intermediate 10

### 7-Chloro-5-fluoro-1H-indazol-6-ol

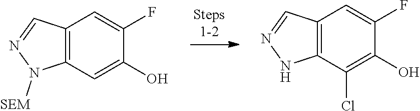 [SMILES: C[Si](C)(C)CCOCn1ncc2cc(F)c(O)cc21 ; Oc1c(F)cc2cn[nH]c2c1Cl]

### Step 1: 7-Chloro-5-fluoro-1-((2-(trimethylsilyl)ethoxy)methyl)-1H-indazol-6-ol

N-Chlorosuccinimide (2.60 g, 19.48 mmol) was added to a solution of Intermediate 9, Step 2 (5.00 g, 17.71 mmol) in dioxane (35 mL) at rt. The mixture was stirred at 60° C. overnight, allowed to cool to rt, poured into H₂O (50 mL), and then extracted with EtOAc (3×60 mL). The combined organic layers were washed with brine (2×100 mL), dried over Na₂SO₄, filtered, concentrated, and then purified by column chromatography (petroleum ether/ethyl acetate=20/1 to 5/1) to give 7-chloro-5-fluoro-1-((2-(trimethylsilyl)ethoxy)methyl)-1H-indazol-6-ol (3.50 g, 62%) as a yellow solid. ¹H NMR (400 MHz, DMSO-d₆): δ 10.65 (s, 1H), 8.07 (s, 1H), 7.60 (d, 1H), 5.88 (s, 2H), 3.49 (t, 2H), 0.76 (t, 2H), −0.13 (s, 9H); LCMS: 317.1 [M+H]⁺.

### Step 2: 7-Chloro-5-fluoro-1H-indazol-6-ol

Concentrated hydrochloric acid (42 mL, ˜12 M) was added to a solution of 7-chloro-5-fluoro-1-((2-(trimethylsilyl)ethoxy)methyl)-1H-indazol-6-ol (3.50 g, 11.05 mmol) in AcOH (20 mL). The mixture was stirred at rt for 3 h, concentrated, adjusted to pH=7 with saturated NaHCO₃ (˜40 mL), and then extracted with EtOAc (3×40 mL). The combined organic layers were washed with brine (2×50 mL), dried over Na₂SO₄, filtered, concentrated, and then purified by column chromatography (petroleum ether/ethyl acetate=10/1 to 1/1) to give 7-chloro-5-fluoro-1H-indazol-6-ol (1.70 g, 82%) as a yellow solid. ¹H NMR (400 MHz, DMSO-d₆): δ 13.27 (s, 1H), 10.47 (s, 1H), 8.02 (s, 1H), 7.53 (d, 1H); LCMS: 187.1 [M+H]⁺.

### Intermediate 11

### 5-Fluoro-4-(trifluoromethyl)-1H-imidazol-6-ol

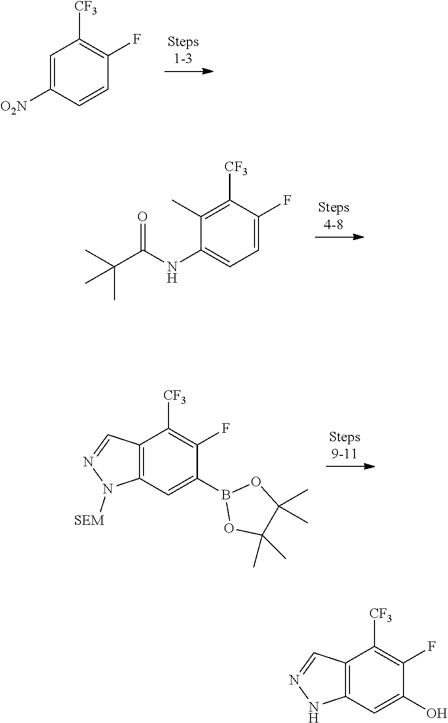 [SMILES: O=[N+]([O-])c1ccc(F)c(C(F)(F)F)c1 ; Cc1c(NC(=O)C(C)(C)C)ccc(F)c1C(F)(F)F ; CC1(C)OB(c2cc3c(cnn3COCC[Si](C)(C)C)c(C(F)(F)F)c2F)OC1(C)C ; Oc1cc2[nH]ncc2c(C(F)(F)F)c1F]

### Step 1: 4-Fluoro-3-(trifluoromethyl)aniline

1-Fluoro-4-nitro-2-(trifluoromethyl)benzene (100 g, 478 mmol) was added to a mixture of 10% Pd/C (10 g, 9.4 mmol) and AcOH (500 mL). The mixture was stirred under H₂ (50 psi) at rt overnight and then filtered through Celite. The filtrate was concentrated under vacuum to give 4-fluoro-3-(trifluoromethyl)aniline (100 g) as a yellow solid. ¹H NMR (400 MHz, DMSO-d₆): δ 7.11-7.06 (m, 1H), 6.86-6.79 (m, 2H), 5.49 (s, 2H); LCMS: 180.1 [M+H]⁺.

### Step 2: N-(4-Fluoro-3-(trifluoromethyl)phenyl)pivalamide

2,2-Dimethylpropanoyl chloride (74.05 g, 614 mmol) was added to a solution of 4-fluoro-3-(trifluoromethyl)aniline (100 g, 558 mmol), TEA (62.14 g, 614 mmol), and THF (1000 mL) at 0° C. The mixture was stirred at 0° C. for 1 h and then filtered. The filtrate was concentrated and then purified by column chromatography (petroleum ether/ethyl acetate=20/0 to 5/1) to give N-(4-fluoro-3-(trifluoromethyl)phenyl)pivalamide (85 g, 67% over two steps) as a yellow solid. ¹H NMR (400 MHz, DMSO-d₆): δ 9.53 (s, 1H), 8.12 (d, 1H), 7.99-7.96 (m, 1H), 7.46-7.41 (m, 1H), 1.22 (s, 9H); LCMS: 264.1 [M+H]⁺.

### Step 3: N-(4-Fluoro-2-methyl-3-(trifluoromethyl)phenyl)pivalamide

n-Butyllithium solution (219 mL, 2.5 M in THF, 548 mmol) was added to a solution of N-(4-fluoro-3-(trifluoromethyl)phenyl)pivalamide (60 g, 228 mmol) in THF (600 mL) at 0° C. The mixture was stirred for 3 h. A solution of iodomethane (32.35 g, 228 mmol) in THF (120 mL) was added dropwise into the reaction mixture at 0° C. The mixture was stirred for 3 h, poured into sat. NH₄Cl (500 mL), and then extracted with EtOAc (3×300 mL). The combined organic layers were dried over Na₂SO₄, concentrated, and then purified by column chromatography (petroleum ether/ethyl acetate=20/0 to 5/1) to give N-(4-fluoro-2-methyl-3-(trifluoromethyl)phenyl)pivalamide (40 g, 63%) as a yellow solid. ¹H NMR (400 MHz, DMSO-d₆): δ 9.18 (s, 1H), 7.46-7.43 (m, 1H), 7.31-7.26 (m, 1H), 2.24-2.22 (m, 3H), 1.23 (s, 9H); LCMS: 278.2 [M+H]⁺.

### Step 4: 4-Fluoro-2-methyl-3-(trifluoromethyl)aniline

Aqueous hydrochloric acid (250 mL, 12 M, 3 mol) was added to a solution of N-(4-fluoro-2-methyl-3-(trifluoromethyl)phenyl)pivalamide (40 g, 144 mmol) in AcOH (250 mL). The mixture was refluxed overnight, allowed to cool to rt, concentrated, and then diluted with water (100 mL). The pH was adjusted to pH˜7 with 4 M NaOH. The mixture was extracted with CH₂Cl₂ (3×200 mL). The combined organic layers were washed with brine (200 mL), dried over Na₂SO₄, filtered, concentrated, and then purified by column chromatography (petroleum ether/ethyl acetate=20/0 to 5/1) to give 4-fluoro-2-methyl-3-(trifluoromethyl)aniline (20 g, 72%) as a red solid. ¹H NMR (400 MHz, DMSO-d₆): δ 6.98-6.88 (m, 2H), 5.14 (s, 2H), 2.13-2.12 (m, 3H); LCMS: 194.1 [M+H]⁺.

### Step 5: 1-(5-Fluoro-4-(trifluoromethyl)-1H-indazol-1-yl)ethanone

Acetic anhydride (30 mL, 311 mmol) was added to a solution of 4-fluoro-2-methyl-3-(trifluoromethyl)aniline (20 g, 104 mmol) in CHCl₃ (150 mL) at 0° C. The mixture was stirred for 30 min and then allowed to warm to rt. 18-Crown-6 (13.69 g, 51.78 mmol), KOAc (30.49 g, 310.65 mmol), and then isopentyl nitrite (24.26 g, 207.10 mmol) were added to the reaction mixture at rt. The mixture was stirred at 85° C. overnight, allowed to cool to rt, concentrated, and then diluted with water (100 mL) and EtOAc (100 mL). The layers were separated. The aqueous layer was extracted with EtOAc (2×100 mL). The combined organic layers were washed with brine, dried over Na₂SO₄, concentrated, and then purified by column chromatography (petroleum ether/ethyl acetate=20/0 to 5/1) to give 1-(5-fluoro-4-(trifluoromethyl)-1H-indazol-1-yl)ethanone (10 g, 39%) as a white solid. ¹HNMR (400 MHz, DMSO-d₆): δ 8.60 (d, 1H), 8.55 (d, 1H), 7.77-7.72 (m, 1H), 2.74 (s, 3H); LCMS: 247.1 [M+H]⁺.

### Step 6: 5-Fluoro-4-(trifluoromethyl)-1H-indazole

Aqueous hydrochloric acid (37%, 40 mL, 414 mmol) was added to a solution of 1-(5-fluoro-4-(trifluoromethyl)-1H-indazol-1-yl)ethanone (10 g, 41 mmol) in CH₃OH (40 mL). The mixture was stirred at 95° C. for 2 h, allowed to cool to rt, concentrated, diluted with H₂O (40 mL), adjusted to pH=7 with sat. NaHCO₃, and then extracted with EtOAc (3×50 mL). The combined organic layers were washed with brine (2×100 mL), dried over Na₂SO₄, filtered, and then concentrated to give 5-fluoro-4-(trifluoromethyl)-1H-indazole (7.60 g) as a yellow solid. ¹H NMR (400 MHz, DMSO-d₆): δ 13.75 (s, 1H), 8.20 (s, 1H), 7.95 (d, 1H), 7.50-7.45 (m, 1H); LCMS: 205.1 [M+H]⁺.

### Step 7: 5-Fluoro-4-(trifluoromethyl)-1-((2-(trimethylsilyl)ethoxy)methyl)-1H-indazole

Sodium hydride (60%, 1.32 g, 33.1 mmol) was added to a solution of 5-fluoro-4-(trifluoromethyl)-1H-indazole (4.5 g, 22 mmol) in DMF (90 mL) at 0° C. The mixture was stirred for 1 h. SEM-Cl (7.35 g, 44.09 mmol) was added at 0° C. The mixture was allowed to warm to rt overnight, poured into ice water (100 mL), and then extracted with EtOAc (3×100 mL). The combined organic layers were washed with water (2×50 mL), washed with brine (50 mL), dried (Na₂SO₄), filtered, concentrated, and then purified by silica gel chromatography (petroleum ether/EtOAc=10/1) to give 5-fluoro-4-(trifluoromethyl)-1-((2-(trimethylsilyl)ethoxy)methyl)-1H-indazole (7 g, 95%) as a yellow solid. ¹H NMR (400 MHz, DMSO-d₆): δ 8.38 (d, 1H), 8.31 (dd, 1H), 7.73 (t, 1H), 5.97 (s, 2H), 3.67-3.55 (m, 2H), 0.95-0.86 (m, 2H), 0.01 (s, 9H); LCMS: 335.1 [M+H]⁺.

### Step 8: 5-Fluoro-6-(4,4,5,5-tetramethyl-1,3,2-dioxaborolan-2-yl)-4-(trifluoromethyl)-1-((2-(trimethylsilyl)ethoxy)methyl)-1H-indazole

A mixture of 5-fluoro-4-(trifluoromethyl)-1-((2-(trimethylsilyl)ethoxy)methyl)-1H-indazole (7 g, 20.93 mmol), 4-tert-butyl-2-(4-tert-butyl-2-pyridyl)pyridine (280.9 mg, 1.05 mmol), 4,4,5,5-tetramethyl-2-(4,4,5,5-tetramethyl-1,3,2-dioxaborolan-2-yl)-1,3,2-dioxaborolane (10.63 g, 41.87 mmol), (1,5-cyclooctadiene)(methoxy)iridium(I) dimer (277.5 mg, 0.418 mmol), and THF (140 mL) was stirred at 80° C. for 2 h under N₂, allowed to cool to rt, concentrated to dryness, and then purified by silica gel chromatography (petroleum ether/EtOAc=20/1) to give 5-fluoro-6-(4,4,5,5-tetramethyl-1,3,2-dioxaborolan-2-yl)-4-(trifluoromethyl)-1-((2-(trimethylsilyl)ethoxy)methyl)-1H-indazole (9 g) as a yellow solid. ¹H NMR (400 MHz, DMSO-d₆): δ 8.44 (d, 1H), 8.40 (s, 1H), 6.03 (s, 2H), 3.73-3.54 (m, 2H), 1.47 (s, 12H), 0.99-0.88 (m, 2H), 0.00 (s, 9H); LCMS: 461.3 [M+H]⁺.

### Step 9: 5-Fluoro-4-(trifluoromethyl)-1-((2-(trimethylsilyl)ethoxy)methyl)-1H-indazol-6-ol

A mixture of 5-fluoro-6-(4,4,5,5-tetramethyl-1,3,2-dioxaborolan-2-yl)-4-(trifluoromethyl)-1-((2-(trimethylsilyl)ethoxy)methyl)-1H-indazole (9 g, 19.55 mmol), NaBO₃-4H₂O (12.03 g, 78.20 mmol), THF (100 mL), and CH₃OH (50 mL) was stirred at rt overnight, poured into water (200 mL), and then extracted with EtOAc (2×150 mL). The combined organic layers were washed with water (2×100 mL), washed with brine (100 mL), dried (Na₂SO₄), filtered, concentrated, and then purified by silica gel chromatography (petroleum ether/EtOAc=15/1) to give 5-fluoro-4-(trifluoromethyl)-1-((2-(trimethylsilyl)ethoxy)methyl)-1H-indazol-6-ol (3.8 g, 56%) as a yellow solid. ¹H NMR (400 MHz, DMSO-d₆): δ 11.11 (s, 1H), 8.19 (d, 1H), 7.55 (d, 1H), 5.82 (s, 2H), 3.59 (t, 2H), 0.90 (t, 2H), 0.00 (s, 9H); LCMS: 351.1 [M+H]⁺.

### Step 10: 5-Fluoro-1-(hydroxymethyl)-4-(trifluoromethyl)-1H-indazol-6-ol

Trifluoroacetic acid (58.52 g, 513.2 mmol) was added to a solution of 5-fluoro-4-(trifluoromethyl)-1-((2-(trimethylsilyl)ethoxy)methyl)-1H-indazol-6-ol (1.9 g, 5.42 mmol) in DCE (60 mL) at rt. The mixture was stirred at rt overnight and concentrated to give 5-fluoro-1-(hydroxymethyl)-4-(trifluoromethyl)-1H-indazol-6-ol (1.4 g) as a yellow solid. LCMS: 251.0 [M+H]⁺.

### Step 11: 5-Fluoro-4-(trifluoromethyl)-1H-indazol-6-ol

Ethane-1,2-diamine (12.6 g, 210 mmol) was added to a solution of 5-fluoro-1-(hydroxymethyl)-4-(trifluoromethyl)-1H-indazol-6-ol (1.4 g, 5.6 mmol) in EtOH (16 mL) at rt. The mixture was stirred at rt overnight, concentrated, adjusted to pH=3 with 1 M HCl, and then extracted with EtOAc (2×100 mL). The combined organic layers were washed with water (2×50 mL), washed with brine (50 mL), dried (Na₂SO₄), filtered, concentrated, and then purified by silica gel chromatography (petroleum ether/EtOAc=1/1) to give 5-fluoro-4-(trifluoromethyl)-1H-indazol-6-ol (900 mg, 73%) as a yellow solid. ¹HNMR (400 MHz, DMSO-d₆): δ 13.20 (s, 1H), 10.78 (s, 1H), 8.02 (s, 1H), 7.29 (d, 1H); LCMS: 221.1 [M+H]⁺.

The Intermediate below was synthesized from 1-fluoro-2,3-dimethyl-4-nitrobenzene following the procedures described for Intermediate 11 (Step 1 and then Steps 5-11).

| Int # | Structure | Name | [M + H]⁺ |
|---|---|---|---|
| 11.1 | 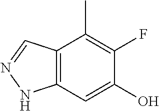 | 5-Fluoro-4-methyl-1H-indazol-6-ol | 167.1 |

### Intermediate 12

### 4-Chloro-5-fluoro-6-methoxy-1H-indazole

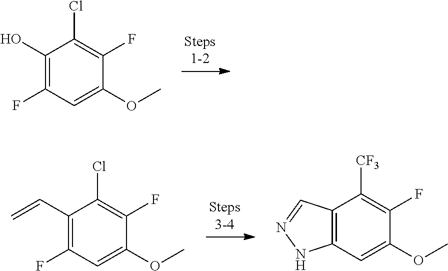 [SMILES: COc1cc(F)c(O)c(Cl)c1F ; C=Cc1c(F)cc(OC)c(F)c1Cl ; COc1cc2[nH]ncc2c(C(F)(F)F)c1F]

### Step 1: 2-Chloro-3,6-difluoro-4-methoxyphenyl trifluoromethanesulfonate

Trifluoromethanesulfonic anhydride (9.43 g, 33.4 mmol) was added to a solution of 2-chloro-3,6-difluoro-4-methoxyphenol (6.5 g, 33.4 mmol), pyridine (2.64 g, 33.4 mmol), and CH₂Cl₂ (100 mL) at 0° C. under N₂. The mixture was stirred at rt for 3 h and then diluted with CH₂Cl₂ (200 mL). The organic layer was washed with water (100 mL), washed with brine (100 mL), dried over Na₂SO₄, filtered, and then concentrated to give 2-chloro-3,6-difluoro-4-methoxyphenyl trifluoromethanesulfonate (9 g) as a yellow oil. ¹H NMR (400 MHz, CDCl₃): δ 6.83 (dd, 1H), 3.94 (s, 3H).

### Step 2: 3-Chloro-1,4-difluoro-5-methoxy-2-vinylbenzene

A mixture of 2-chloro-3,6-difluoro-4-methoxyphenyl trifluoromethanesulfonate (9 g), potassium trifluoro(vinyl)borate (7.38 g, 55.11 mmol), Et₃N (5.58 g, 55.11 mmol), Pd(dppf)Cl₂ (1.01 g, 1.38 mmol), and EtOH (150 mL) was stirred at 80° C. overnight under N₂. The reaction mixture was allowed to cool to rt, poured into water (400 mL), and then extracted with EtOAc (3×100 mL). The combined organic layers were washed with brine (100 mL), dried over Na₂SO₄, filtered, concentrated, and then purified by silica gel chromatography (petroleum ether/EtOAc=95/5) to give 3-chloro-1,4-difluoro-5-methoxy-2-vinylbenzene (3.4 g, 49% over 2 steps) as a yellow oil. ¹H NMR (400 MHz, CDCl₃): δ 6.79-6.60 (m, 2H), 5.97-5.88 (m, 1H), 5.60 (d, 1H), 3.90 (s, 3H).

### Step 3: 2-Chloro-3,6-difluoro-4-methoxybenzaldehyde

An ozone-enriched stream of oxygen (15 psi) was bubbled through a cold (−78° C.) solution of 3-chloro-1,4-difluoro-5-methoxy-2-vinylbenzene (3.2 g, 15.64 mmol) in CH₂Cl₂ (50 mL) until it turned light blue (˜15 min). The solution was purged with N₂ at −78° C. for 15 min to remove excess O₃. The reaction was quenched with triphenylphosphine (10.26 g, 39.10 mmol), stirred at rt overnight, concentrated, and then purified by silica gel chromatography (petroleum ether/EtOAc=97/3) to give 2-chloro-3,6-difluoro-4-methoxybenzaldehyde (1.35 g, 41%) as a white solid. ¹HNMR (400 MHz, DMSO-d₆): δ 10.17 (s, 1H), 7.39 (dd, 1H), 4.02 (s, 3H).

### Step 4: 4-Chloro-5-fluoro-6-methoxy-1H-indazole

Hydrazine monohydrate (5.52 g, 108 mmol, 98%) was added over 5 min to a solution of 2-chloro-3,6-difluoro-4-methoxybenzaldehyde (1.05 g, 5.08 mmol) in NMP (10 mL). The reaction mixture was stirred at 130° C. for 3 h, allowed to cool to rt, poured into water (50 mL), and then extracted with EtOAc (3×20 mL). The combined organic layers were washed with brine (20 mL), dried over Na₂SO₄, filtered, concentrated, and then purified by silica gel chromatography (petroleum ether/EtOAc=3/1) to give 4-chloro-5-fluoro-6-methoxy-1H-indazole (1.3 g) as a yellow oil. ¹HNMR (400 MHz, DMSO-d₆): δ 13.31 (s, 1H), 8.05 (s, 1H), 7.14 (d, 1H), 3.94 (s, 3H); LCMS: 201.0 [M+H]⁺.

### Intermediate 13

### 1-(4-Bromophenyl)-4,7-difluoro-6-(methoxymethoxy)-1H-indazole

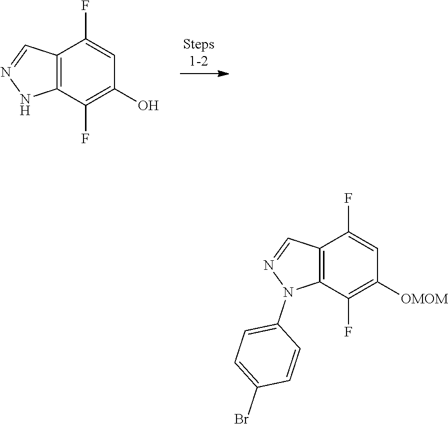

### Step 1: 4,7-Difluoro-6-(methoxymethoxy)-1H-indazole

A mixture of Intermediate 8 (200 mg, 1.18 mmol), K₂CO₃ (325 mg, 2.35 mmol), MOM-Cl (133 mg, 1.65 mmol), and acetone (10 mL) was stirred at rt overnight. The mixture was poured into water (20 mL) and extracted with EtOAc (2×20 mL). The combined organic layers were washed with water (2×10 mL), washed with brine (10 mL), dried (Na₂SO₄), filtered, concentrated, and then purified by silica gel chromatography (petroleum ether/EtOAc=10/1) to give 4,7-difluoro-6-(methoxymethoxy)-1H-indazole (140 mg, 56%) as a yellow solid. LCMS: 215.0 [M+H]⁺.

### Step 2: 1-(4-Bromophenyl)-4,7-difluoro-6-(methoxymethoxy)-1H-indazole

A mixture of 4,7-difluoro-6-(methoxymethoxy)-1H-indazole (140 mg, 0.653 mmol), (4-bromophenyl)boronic acid (197 mg, 0.980 mmol), Cu(OAc)₂ (178.09 mg, 0.980 mmol), pyridine (155 mg, 1.96 mmol), and CH₂Cl₂ (30 mL) was stirred at rt overnight under O₂ (15 psi), poured into water (20 mL), and then extracted with CH₂Cl₂ (2×20 mL). The combined organic layers were washed with water (2×10 mL), washed with brine (10 mL), dried (Na₂SO₄), filtered, concentrated, and then purified by prep-TLC (petroleum ether/EtOAc=4/1) to give 1-(4-bromophenyl)-4,7-difluoro-6-(methoxymethoxy)-1H-indazole (100 mg, 410) as a yellow oil. ¹H NMR (400 MHz, DMSO-d₆): δ 8.53 (d, 1H), 7.77 (d, 2H), 7.62 (d, 2H), 7.19 (d, 1H), 5.33 (s, 2H), 3.40 (s, 3H); LCMS 3690 [M+H]⁺.

The Intermediates below were synthesized from the appropriate starting material or Intermediate and the appropriate boronic acid following the procedures described for Intermediate 13.

| Int | Structure | Name | [M + H]⁺ |
|---|---|---|---|
| 13.1³ | 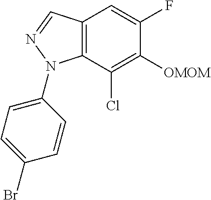 | 1-(4-Bromophenyl)-7-chloro-5- fluoro-6-(methoxymethoxy)-1H- indazole | 385.0 |
| 13.2 | 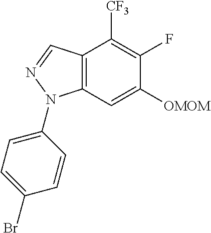 | 1-(4-Bromophenyl)-5-fluoro-6- (methoxymethoxy)-4- (trifluoromethyl)-1H-indazole | 419.0 |
| 13.3⁴ | 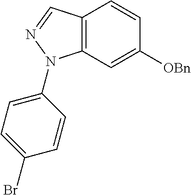 [SMILES: Brc1ccc(-n2ncc3ccc(OCc4ccccc4)cc32)cc1] | 6-(Benzyloxy)-1-(4- bromophenyl)-1H-indazole | 378.9 |
| 13.4^{1,4} | 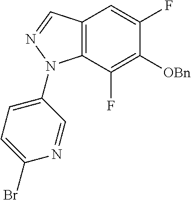 [SMILES: Fc1cc2cnn(-c3ccc(Br)nc3)c2c(F)c1OCc1ccccc1] | 6-(Benzyloxy)-1-(6-bromopyridin- 3-yl)-5,7-difluoro-1H-indazole | 416.0 |
| 13.5⁴ | 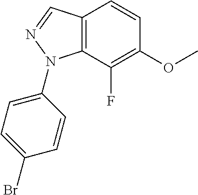 [SMILES: COc1ccc2cnn(-c3ccc(Br)cc3)c2c1F] | 1-(4-Bromophenyl)-7-fluoro-6- methoxy-1H-indazole | 320.8 |
| 13.6⁴ | 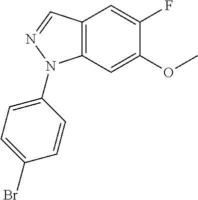 [SMILES: COc1cc2c(cnn2-c2ccc(Br)cc2)cc1F] | 1-(4-Bromophenyl)-5-fluoro-6- methoxy-1H-indazole | 320.9 |
| 13.7⁴ | 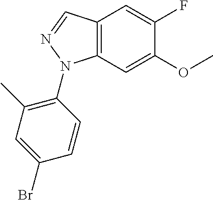 | 1-(4-Bromo-2-methylphenyl)-5- fluoro-6-methoxy-1H-indazole | 334.9 |
| 13.8⁴ | 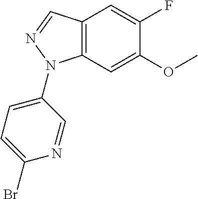 [SMILES: COc1cc2c(cnn2-c2ccc(Br)nc2)cc1F] | 1-(6-Bromopyridin-3-yl)-5-fluoro- 6-methoxy-1H-indazole | 321.8 |
| 13.9^{2,4} | 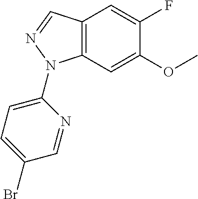 [SMILES: COc1cc2c(cnn2-c2ccc(Br)cn2)cc1F] | 1-(5-Bromopyridin-2-yl)-5-fluoro- 6-methoxy-1H-indazole | 321.8 |
| 13.10 | 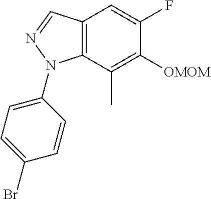 | 1-(4-Bromophenyl)-5-fluoro-6- (methoxymethoxy)-7-methyl-1H- indazole | 365.1 |
| 13.11⁴ | 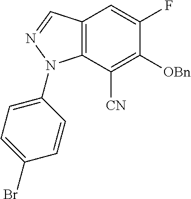 [SMILES: N#Cc1c(OCc2ccccc2)c(F)cc2cnn(-c3ccc(Br)cc3)c12] | 6-(Benzyloxy)-1-(4- bromophenyl)-5-fluoro-1H- indazole-7-carbonitrile | 422.1 |

Alternate conditions:
Step 1: MOM-Cl added at 0° C.
Step 2: 16-64 h; in some instances, additional boronic acid, Cu(OAc)₂, and pyridine were needed for full conversion;
¹DCE, 80° C., ON;
²CH₂Cl₂, rt, 64 h then DMF, 110° C., 41 h.
³Isolated as a mixture of N1/N2 isomers.
⁴Step 2 only.

### Intermediate 14

### 1-(4-Bromophenyl)-5,7-difluoro-6-methoxy-1H-indazole

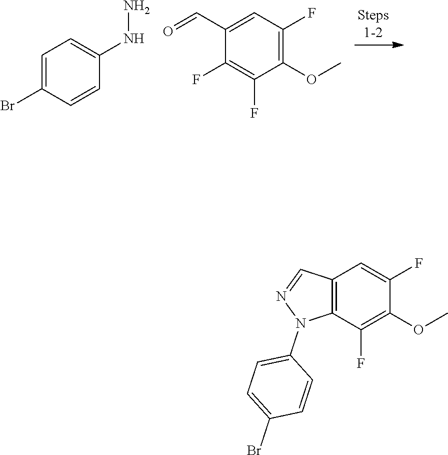

### Step 1: (E)-1-(4-Bromophenyl)-2-(2,3,5-trifluoro-4-methoxybenzylidene)hydrazine

A mixture of Intermediate 1 (500 mg, 2.62 mmol), 4-bromophenylhydrazine (541 mg, 2.89 mmol), and 1,4-dioxane (1 mL) was heated at 80° C. for 4 h, cooled to rt, and then diluted with water (10 mL). The precipitate was filtered, and the filter cake was washed with water (10 mL) to give (E)-1-(4-bromophenyl)-2-(2,3,5-trifluoro-4-methoxybenzylidene)hydrazine (939 mg, 99%) as a reddish brown solid. ¹H NMR (400 MHz, DMSO-d₆): δ 10.85 (s, 1H), 7.93 (s, 1H), 7.60 (ddd, J=2.1, 6.4, 12.1 Hz, 1H), 7.39 (d, J=8.8 Hz, 2H), 7.06 (d, J=8.8 Hz, 2H), 4.01 (s, 3H); LCMS: 359.4 [M+H]⁺.

### Step 2: 1-(4-Bromophenyl)-5,7-difluoro-6-methoxy-1H-indazole

A mixture of (E)-1-(4-bromophenyl)-2-(2,3,5-trifluoro-4-methoxybenzylidene)hydrazine (935 mg, 2.60 mmol), potassium carbonate (395 mg, 2.86 mmol), and 1-methyl-2-pyrrolidinone (3 mL) was heated at 220° C. in a microwave for 15 min, cooled to rt, and then diluted with water/brine (2:1, 30 mL). The aqueous layer was extracted with EtOAc (2×10 mL). The combined organic layers were washed with brine (10 mL), dried (Na₂SO₄), filtered, concentrated, and then purified by silica gel chromatography (0-50% CH₂Cl₂ in hexanes then 0-10% EtOAc in hexanes) to give 1-(4-bromophenyl)-5,7-difluoro-6-methoxy-1H-indazole (517 mg, 59%) as an off-white solid. ¹HNMR (400 MHz, DMSO-d₆): δ 8.42 (d, J=2.3 Hz, 1H), 7.76 (d, J=8.8 Hz, 2H), 7.67 (dd, J=1.3, 9.9 Hz, 1H), 7.65-7.60 (m, 2H), 3.99 (s, 3H); LCMS: 338.8 [M+H]⁺.

The Intermediates below were synthesized from the appropriate Intermediate or starting material and the appropriate hydrazine following the procedures described for Intermediate 14.

| Int | Structure | Name | [M + H]⁺ |
|---|---|---|---|
| 14.1² | 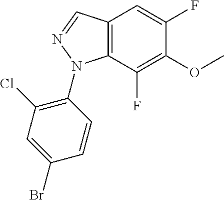 [SMILES: COc1c(F)cc2cnn(-c3ccc(Br)cc3Cl)c2c1F] | 1-(4-Bromo-2-chlorophenyl)-5,7- difluoro-6-methoxy-1H-indazole | 372.8 |
| 14.2² | 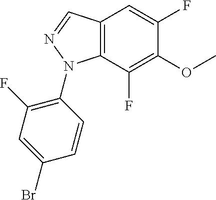 [SMILES: COc1c(F)cc2cnn(-c3ccc(Br)cc3F)c2c1F] | 1-(4-Bromo-2-fluorophenyl)-5,7- difluoro-6-methoxy-1H-indazole | 356.8 |
| 14.3² | 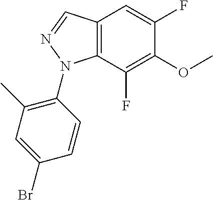 | 1-(4-Bromo-2-methylphenyl)-5,7- difluoro-6-methoxy-1H-indazole | 352.8 |
| 14.4² | 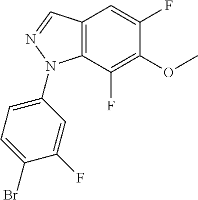 [SMILES: COc1c(F)cc2cnn(-c3ccc(Br)c(F)c3)c2c1F] | 1-(4-Bromo-3-fluorophenyl)-5,7- difluoro-6-methoxy-1H-indazole | 356.8 |
| 14.5² | 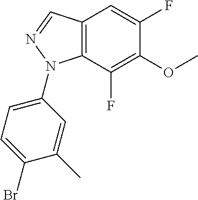 [SMILES: COc1c(F)cc2cnn(-c3ccc(Br)c(C)c3)c2c1F] | 1-(4-Bromo-3-methylphenyl)-5,7- difluoro-6-methoxy-1H-indazole | 352.8 |
| 13.6² | 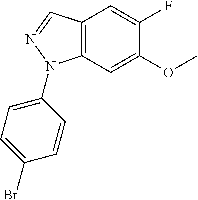 [SMILES: COc1cc2c(cnn2-c2ccc(Br)cc2)cc1F] | 1-(4-Bromophenyl)-5-fluoro-6- methoxy-1H-indazole | 320.8 |
| 14.6 | 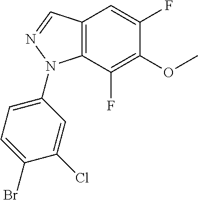 [SMILES: COc1c(F)cc2cnn(-c3ccc(Br)c(Cl)c3)c2c1F] | 1-(4-Bromo-3-chlorophenyl)-5,7- difluoro-6-methoxy-1H-indazole | 373.0 |
| 14.7 | 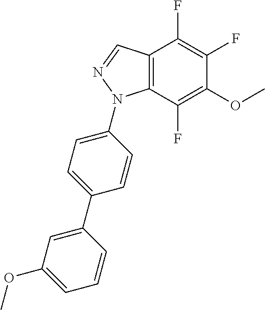 | 4,5,7-Trifluoro-6-methoxy-1-(3′- methoxy-[1,1′-biphenyl]-4-yl)-1H- indazole | 385.1 |
| 14.8¹ | 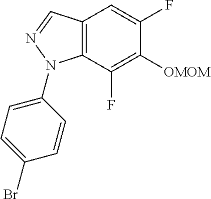 | 1-(4-Bromophenyl)-5,7-difluoro- 6-(methoxymethoxy)-1H-indazole | 368.9 |
| 14.9^{1,3} | 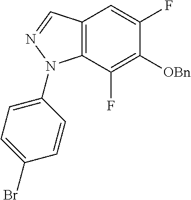 [SMILES: Fc1cc2cnn(-c3ccc(Br)cc3)c2c(F)c1OCc1ccccc1] | 6-(Benzyloxy)-1-(4- bromophenyl)-5,7-difluoro-1H- indazole | 414.9 |

Alternate conditions:
Step 1: CH₃OH or EtOH instead of dioxane; 80-100° C., 3-17 h. ¹rt, 5 min-3 h.
Step 2:
²Microwave, 210-220° C., 3-30 min.
³KOtBu, 2-MeTHF, 90° C., 8 h.

### Intermediate 15

### 1-(4-Bromophenyl)-5,7-difluoro-6-methoxy-1H-benzo[d][1,2,3]triazole

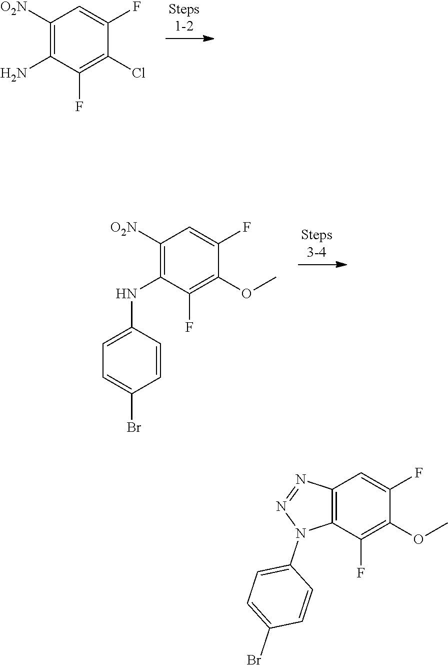 [SMILES: Nc1c([N+](=O)[O-])cc(F)c(Cl)c1F ; COc1c(F)cc([N+](=O)[O-])c(Nc2ccc(Br)cc2)c1F ; COc1c(F)cc2nnn(-c3ccc(Br)cc3)c2c1F]

### Step 1: 2,4-Difluoro-3-methoxy-6-nitroaniline

Sodium methoxide (0.5 M in methanol, 57.5 mL, 28.77 mmol) was added to 3-chloro-2,4-difluoro-6-nitrophenylamine (2.00 g, 9.59 mmol) in a pressure vessel. The vessel was sealed, heated at 90° C. for 2 h, allowed to cool to rt, and then diluted with water (50 mL). Methanol was removed, and the remaining mixture was diluted with water (50 mL). The precipitate was filtered. The filter cake was washed with water (50 mL) and then purified by silica gel chromatography (0-80% CH₂Cl₂ in hexanes) to give 2,4-difluoro-3-methoxy-6-nitroaniline (1.46 g, 74%) as a bright yellow solid. ¹H NMR (400 MHz, DMSO-d₆): δ 7.78 (d, J=12.3 Hz, 1H), 7.32 (br s, 2H), 4.09 (s, 3H); LCMS: 205.0 [M+H]⁺.

### Step 2: N-(4-Bromophenyl)-2,4-difluoro-3-methoxy-6-nitroaniline

Copper(II) acetate (1.87 g, 10.3 mmol) was added to a mixture of 2,4-difluoro-3-methoxy-6-nitroaniline (700 mg, 3.43 mmol), 4-bromophenylboronic acid (2.11 g, 10.3 mmol), triethylamine (1.5 mL, 10.4 mmol), and CH₂Cl₂ (14 mL) at rt. The mixture was stirred vigorously at rt for 21 h. Celite was added, and the reaction was filtered through Celite. The filter cake was washed with CH₂Cl₂ (30 mL). The filtrate was concentrated and then purified by silica gel chromatography (0-40% CH₂Cl₂ in hexanes) to give N-(4-bromophenyl)-2,4-difluoro-3-methoxy-6-nitroaniline (637 mg, 52%) as an orange solid. ¹H NMR (400 MHz, DMSO-d₆): δ 8.58 (s, 1H), 8.02 (d, J=11.6 Hz, 1H), 7.36 (d, J=8.4 Hz, 2H), 6.83 (br d, J=8.2 Hz, 2H), 4.09 (s, 3H); LCMS: 359.3 [M+H]⁺.

### Step 3: N¹-(4-Bromophenyl)-4,6-difluoro-5-methoxybenzene-1,2-diamine

A mixture of N-(4-bromophenyl)-2,4-difluoro-3-methoxy-6-nitroaniline (637 mg, 1.77 mmol), tin(II) chloride dihydrate (2.00 g, 8.87 mmol), and EtOH (10 mL) was heated at 70° C. for 2 h, allowed to cool to rt, concentrated, and then diluted with EtOAc (10 mL). Aqueous sodium hydroxide (1.0 M, 15 mL) was added, and the mixture was stirred at rt for 20 min. Celite was added, and the reaction was filtered through Celite. The filter cake washed with EtOAc (3×20 mL) and then concentrated to give N-(4-bromophenyl)-4,6-difluoro-5-methoxybenzene-1,2-diamine (586 mg, 95%) as an orange liquid. ¹H NMR (400 MHz, DMSO-d₆): δ 7.32 (s, 1H), 7.25 (d, J=8.6 Hz, 2H), 6.49-6.41 (m, 3H), 5.18 (br s, 2H), 3.74 (s, 3H); LCMS: 328.8 [M+H]⁺.

### Step 4: 1-(4-Bromophenyl)-5,7-difluoro-6-methoxy-LH-benzo[d][1,2,3]triazole

Concentrated sulfuric acid (1 mL, 18.8 mmol) and water (9 mL) were added to N¹-(4-bromophenyl)-4,6-difluoro-5-methoxybenzene-1,2-diamine (320 mg, 0.97 mmol) in THE (3 mL) at 0° C. Sodium nitrite (94 mg, 1.36 mmol) in water (1 mL) was added dropwise. The mixture was stirred at 0° C. for 15 min, poured into water (30 mL), and then extracted with EtOAc (3×10 mL). The combined organic layers were washed with water (20 mL), washed with brine (20 mL), dried (Na₂SO₄), filtered, concentrated, and then purified by silica gel chromatography (0-10% EtOAc in hexane) to give 1-(4-bromophenyl)-5,7-difluoro-6-methoxy-1H-benzo[d][1,2,3]triazole (192 mg, 58%) as a light orange solid. 1HNMR (400 MHz, DMSO-d₆): δ 8.09 (d, J=9.8 Hz, 1H), 7.91-7.86 (m, 2H), 7.82-7.76 (m, 2H), 4.04 (s, 3H); LCMS: 339.8 [M+H]⁺.

The Intermediate below was synthesized from 4-fluoro-5-methoxy-2-nitrophenylamine following the procedures described for Intermediate 15, Steps 2-4.

| Cmpd | Structure | Name | [M + H]⁺ |
|---|---|---|---|
| 15.1 | 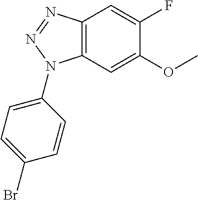 [SMILES: COc1cc2c(cc1F)nnn2-c1ccc(Br)cc1] | 1-(4-Bromophenyl)-5-fluoro-6- methoxy-1H- benzo[d][1,2,3]triazole | 321.8 |

### Intermediate 15.2

### 6-(Benzyloxy)-1-(4-bromophenyl)-5,7-difluoro-1H-benzo[d][1,2,3]triazole

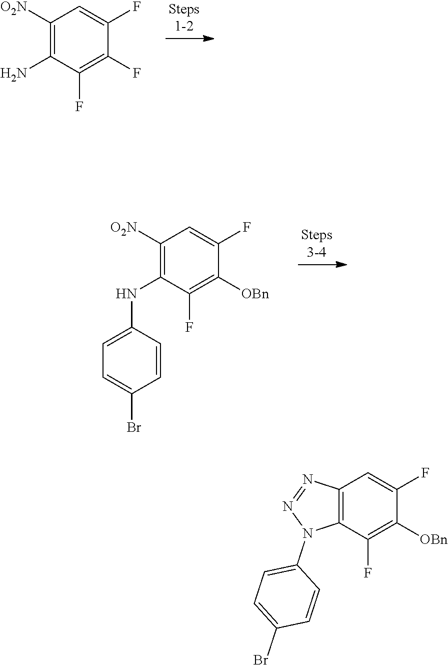 [SMILES: Nc1c([N+](=O)[O-])cc(F)c(F)c1F ; O=[N+]([O-])c1cc(F)c(OCc2ccccc2)c(F)c1Nc1ccc(Br)cc1 ; Fc1cc2nnn(-c3ccc(Br)cc3)c2c(F)c1OCc1ccccc1]

### Step 1: 3-(Benzyloxy)-2,4-difluoro-6-nitroaniline

Sodium hydride (3.12 g, 78.1 mmol, 60% in mineral oil) was added to a solution of 2,3,4-trifluoro-6-nitroaniline (5.00 g, 26.0 mmol) in THF (100 mL) at 0° C. under N₂. The mixture was stirred for 0.5 h. Benzyl alcohol (4.06 mL, 39.0 mmol) in THF (10 mL) was added dropwise at 0° C. The reaction mixture was stirred at rt for 1 h, poured into ice-water (100 mL) carefully, and then extracted with EtOAc (3×100 mL). The combined organic phases were washed with brine (100 mL), dried over Na₂SO₄, filtered, concentrated, and then triturated with EtOH (20 mL) for 30 min to obtain 3-(benzyloxy)-2,4-difluoro-6-nitroaniline (5.00 g, 69%) as a yellow solid. ¹HNMR (400 MHz, DMSO-d₆): δ 7.78 (d, 1H), 7.45-7.36 (m, 5H), 7.29 (s, 2H), 5.35 (s, 2H).

### Step 2: 3-(Benzyloxy)-N-(4-bromophenyl)-2,4-difluoro-6-nitroaniline

Tris(dibenzylideneacetone)dipalladium(0) (1.63 g, 1.78 mmol) was added to a solution of 3-(benzyloxy)-2,4-difluoro-6-nitroaniline (5.00 g, 17.8 mmol), 1-bromo-4-iodobenzene (6.06 g, 21.4 mmol), XantPhos (2.06 g, 3.57 mmol), and NaOtBu (3.43 g, 35.7 mmol) in toluene (80 mL) under N₂. The mixture was degassed and purged with N₂ 3 times, stirred at 100° C. overnight, allowed to cool to rt, poured into H₂O (80 mL), and then extracted with EtOAc (3×80 mL). The combined organic layers were washed with brine (2×50 mL), dried over Na₂SO₄, filtered, concentrated, and then purified by column chromatography (petroleum ether/ethyl acetate=20/1 to 1/1) to obtain 3-(benzyloxy)-N-(4-bromophenyl)-2,4-difluoro-6-nitroaniline (3.2 g ˜80% ¹H NMR purity and 3.0 g ˜50% ¹H NMR purity) as a yellow solid. ¹H NMR (400 MHz, DMSO-d₆): δ 8.53 (s, 1H), 8.01 (d, 1H), 7.43-7.38 (m, 5H), 7.34 (d, 2H), 6.70 (d, 2H), 5.35 (s, 2H); LCMS: 435.0 [M+H]⁺.

### Step 3: 5-(Benzyloxy)-N1-(4-bromophenyl)-4,6-difluorobenzene-1,2-diamine

Iron powder (2.46 g, 44.1 mmol) was added to a solution of 3-(benzyloxy)-N-(4-bromophenyl)-2,4-difluoro-6-nitroaniline (3.20 g, 7.35 mmol), NH₄Cl (2.36 g, 44.1 mmol), EtOH (40 mL), and H₂O (10 mL). The mixture was stirred at 80° C. for 1 h, allowed to cool to rt, and then filtered. The filtrate was concentrated under vacuum to obtain 5-(benzyloxy)-N1-(4-bromophenyl)-4,6-difluorobenzene-1,2-diamine (3.00 g) as a red oil. ¹H NMR (400 MHz, DMSO-d₆): δ 7.37-7.36 (m, 5H), 7.24-7.20 (m, 3H), 6.41-6.35 (m, 3H), 5.15 (s, 2H), 4.95 (s, 2H); LCMS: 405.0 [M+H]⁺.

### Step 4: 6-(Benzyloxy)-1-(4-bromophenyl)-5,7-difluoro-LH-benzo[d][1,2,3]triazole

Sulfuric acid (7.50 mL, 141 mmol) in H₂O (55 mL) and then NaNO₂ (715 mg, 10.4 mmol) in H₂O (5 mL) were added to a solution of 5-(benzyloxy)-N1-(4-bromophenyl)-4,6-difluorobenzene-1,2-diamine (3.00 g) in THF (30 mL) at 0° C. The mixture was stirred for 1 h, allowed to warm slowly to rt, poured into H₂O (20 mL), and then extracted with EtOAc (3×30 mL). The combined organic layers were washed with brine (2×20 mL), dried over Na₂SO₄, filtered, concentrated, and then purified by column chromatography (petroleum ether/ethyl acetate=20/1 to 5/1) to obtain 6-(benzyloxy)-1-(4-bromophenyl)-5,7-difluoro-1H-benzo[d][1,2,3]triazole (2.20 g, 89% over two steps) as a red solid. ¹HNMR (400 MHz, DMSO-d₆): δ 8.08 (d, 1H), 7.88 (d, 2H), 7.77 (d, 2H), 7.46-7.44 (m, 2H), 7.41-7.36 (m, 3H), 5.26 (s, 2H); LCMS: 416.0 [M+H]⁺.

The Intermediates below were synthesized from the appropriate starting materials following the procedures described for Intermediate 15.2.

| Int | Structure | Name | [M + H]⁺ |
|---|---|---|---|
| 15.3^{1,4} | 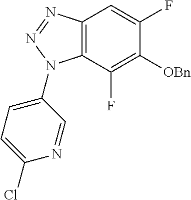 [SMILES: Fc1cc2nnn(-c3ccc(Cl)nc3)c2c(F)c1OCc1ccccc1] | 6-(Benzyloxy)-1-(6-chloropyridin- 3-yl)-5,7-difluoro-1H- benzo[d][1,2,3]triazole | 373.0 |
| 15.4^{1,4} | 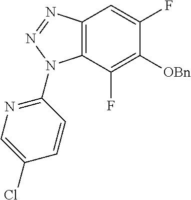 [SMILES: Fc1cc2nnn(-c3ccc(Cl)cn3)c2c(F)c1OCc1ccccc1] | 6-(Benzyloxy)-1-(5-chloropyridin- 2-yl)-5,7-difluoro-1H- benzo[d][1,2,3]triazole | 373.1 |
| 15.5¹ | 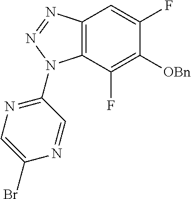 [SMILES: Fc1cc2nnn(-c3cnc(Br)cn3)c2c(F)c1OCc1ccccc1] | 6-(Benzyloxy)-1-(5- bromopyrazin-2-yl)-5,7-difluoro- 1H-benzo[d][1,2,3]triazole | 417.9 |
| 15.6² | 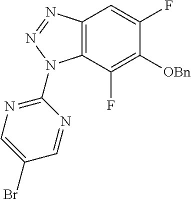 [SMILES: Fc1cc2nnn(-c3ncc(Br)cn3)c2c(F)c1OCc1ccccc1] | 6-(Benzyloxy)-1-(5- bromopyrimidin-2-yl)-5,7- difluoro-1H- benzo[d][1,2,3]triazole | 418.0 |
| 15.7³ | 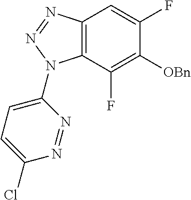 [SMILES: Fc1cc2nnn(-c3ccc(Cl)nn3)c2c(F)c1OCc1ccccc1] | 6-(Benzyloxy)-1-(6- chloropyridazin-3-yl)-5,7-difluoro- 1H-benzo[d][1,2,3]triazole | 374.1 |

Alternate conditions:
Step 2:
¹Pd₂(dba)₃, XantPhos, Cs₂CO₃, toluene, 100° C, 3 h-ON;
²Cs₂CO₃, DMF, rt, ON; ³Cs₂CO₃, NMP, 50° C., ON;
Step 3:
⁴SnCl₂, EtOH, H₂O, 70° C., 2 h.

### Intermediate 16

### 5,7-Difluoro-1-(4-(4-(methylsulfonyl)piperazin-1-yl)phenyl)-1H-benzo[d]imidazol-6-ol

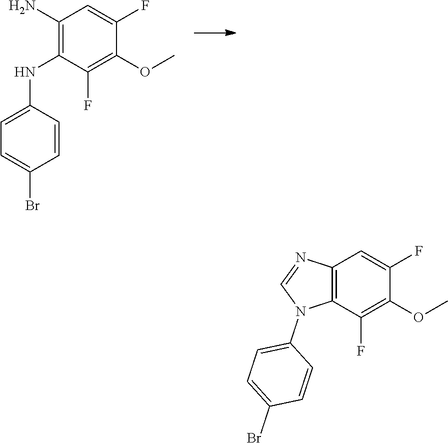 [SMILES: COc1c(F)cc(N)c(Nc2ccc(Br)cc2)c1F ; COc1c(F)cc2ncn(-c3ccc(Br)cc3)c2c1F]

A mixture of Intermediate 15, Step 3 (281 mg, 0.81 mmol), formic acid (0.3 mL, 8.0 mmol), 1,4-dioxane (0.6 mL), and water (0.6 mL) was stirred at 100° C. for 3 h, allowed to cool to rt, and then diluted with water (12 mL). The mixture was stirred at rt for 10 min and then filtered. The filter cake was washed with water (2×10 mL) to give 1-(4-bromophenyl)-5,7-difluoro-6-methoxy-1H-benzo[d]imidazole (260 mg, 94%) as a light purple solid. ¹H NMR (400 MHz, DMSO-d₆): δ 8.51 (s, 1H), 7.80 (d, J=8.3 Hz, 2H), 7.67-7.58 (m, 3H), 3.92 (s, 3H); LCMS: 338.8 [M+H]⁺.

The Intermediate below was synthesized from 4-fluoro-5-methoxy-2-nitrophenylamine following the synthesis of Intermediate 16.

| Cmpd | Structure | Name | [M + H]⁺ |
|---|---|---|---|
| 16.1 | 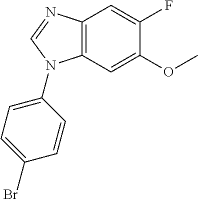 [SMILES: COc1cc2c(cc1F)ncn2-c1ccc(Br)cc1] | 1-(4-Bromophenyl)-5-fluoro-6- methoxy-1H-benzo[d]imidazole | 320.9 |

### Intermediate 17

### 1-(4-Bromophenyl)-5,7-difluoro-6-methoxy-2-methyl-1H-benzo[d]imidazole

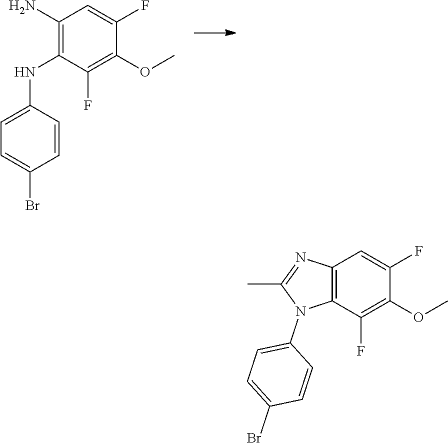 [SMILES: COc1c(F)cc(N)c(Nc2ccc(Br)cc2)c1F ; COc1c(F)cc2nc(C)n(-c3ccc(Br)cc3)c2c1F]

Acetyl chloride (68 μL, 0.96 mmol) was added to Intermediate 15, Step 3 (150 mg, 0.46 mmol) in toluene (1 mL) at 0° C. The reaction was stirred at 115° C. for 1 h, allowed to cool to rt, and then diluted with CH₂Cl₂ (10 mL). The organic layer was washed with 1.0 M NaOH (10 mL), washed with brine (10 mL), dried (Na₂SO₄), filtered, concentrated, and then purified by silica gel chromatography (0-25% EtOAc in CH₂Cl₂) to give 1-(4-bromophenyl)-5,7-difluoro-6-methoxy-2-methyl-1H-benzo[d]imidazole (129 mg, 80%) as a tan solid. 1H NMR (400 MHz, DMSO-d₆): δ 7.80 (d, J=8.4 Hz, 2H), 7.60 (d, J=8.1 Hz, 2H), 7.43 (d, J=10.8 Hz, 1H), 3.86 (s, 3H), 2.35 (s, 3H); LCMS: 352.8 [M+H]⁺.

### Intermediate 18

### 1-(4-Bromophenyl)-5,7-difluoro-6-methoxy-1H-benzo[d]imidazol-2(3H)-one

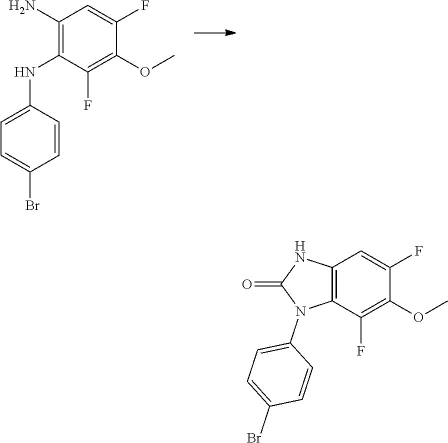 [SMILES: COc1c(F)cc(N)c(Nc2ccc(Br)cc2)c1F ; COc1c(F)cc2[nH]c(=O)n(-c3ccc(Br)cc3)c2c1F]

Pyridine (0.25 mL, 3.09 mmol) was added to Intermediate 15, Step 3 (500 mg, 1.52 mmol) and 1,1′-carbonyldiimidazole (492 mg, 3.03 mmol) in THF (2.5 mL). The reaction was stirred at 65° C. for 1 h, cooled to rt, concentrated, and then diluted with EtOAc (10 mL) and hexanes (30 mL). The mixture was sonicated, warmed with a heat gun to achieve dissolution, and then cooled to rt. The solids were filtered, and the filter cake was washed with a mixture of 1:3 EtOAc/hexanes (20 mL) to give 1-(4-bromophenyl)-5,7-difluoro-6-methoxy-1H-benzo[d]imidazol-2(3H)-one (448 mg, 83%) as an off-white solid. ¹H NMR (400 MHz, DMSO-d₆): δ 11.49 (s, 1H), 7.71 (d, J=8.6 Hz, 2H), 7.47 (br d, J=7.6 Hz, 2H), 6.92 (d, J=9.9 Hz, 1H), 3.82 (s, 3H); LCMS: 354.7 [M+H]⁺.

### Intermediate 19

### 1-(4-Bromophenyl)-2-chloro-5,7-difluoro-6-methoxy-1H-benzo[d]imidazole

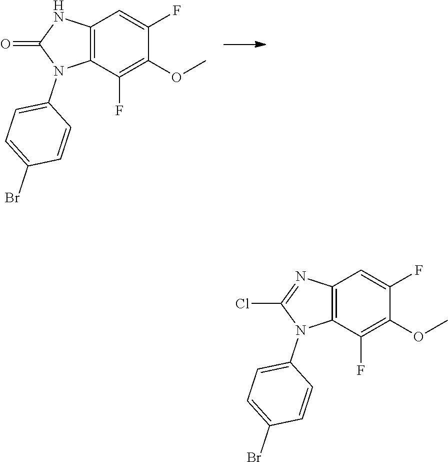 [SMILES: COc1c(F)cc2[nH]c(=O)n(-c3ccc(Br)cc3)c2c1F ; COc1c(F)cc2nc(Cl)n(-c3ccc(Br)cc3)c2c1F]

Phosphorous (V) oxychloride (1 mL) was added to Intermediate 18 (100 mg, 0.28 mmol). The reaction was stirred at 100° C. for 5 h, cooled to rt, poured into ice water (10 mL), basified to pH˜14 with 1.0 M NaOH, and then extracted with EtOAc (10 mL). The organic layer was washed with brine (10 mL), dried (Na₂SO₄), filtered, and then concentrated. The crude product was suspended in minimal EtOAc, sonicated for 1 min, diluted with hexanes (1 mL), and then filtered to remove residual SM. The filtrate was concentrated to give 1-(4-bromophenyl)-2-chloro-5,7-difluoro-6-methoxy-1H-benzo[d]imidazole (69 mg, 58%, 88% pure) as a pale pink sticky solid. ¹H NMR (400 MHz, DMSO-d₆): δ 7.84 (d, J=8.6 Hz, 2H), 7.67 (d, J=8.2 Hz, 2H), 7.59 (d, J=10.4 Hz, 1H), 3.89 (s, 3H); LCMS: 372.7 [M+H]⁺.

### Intermediate 20

### 3-(4-Bromophenyl)-4,6-difluoro-5-methoxy-1-methyl-1H-benzo[d]imidazol-2(3H)-one

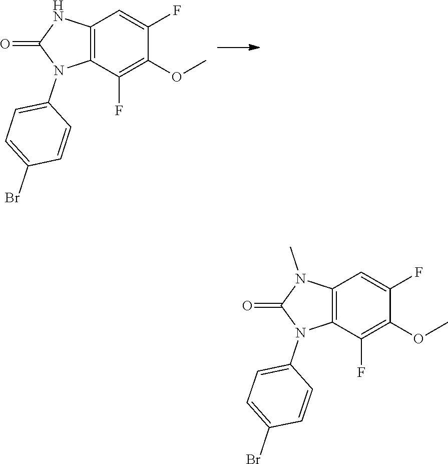 [SMILES: COc1c(F)cc2[nH]c(=O)n(-c3ccc(Br)cc3)c2c1F ; COc1c(F)cc2c(c1F)n(-c1ccc(Br)cc1)c(=O)n2C]

Iodomethane (20 μL, 0.32 mmol) was added to Intermediate 18 (75 mg, 0.21 mmol) and cesium carbonate (137 mg, 0.42 mmol) in DMF (1 mL). The reaction was stirred at rt for 30 min and diluted with water (6 mL). The precipitate was filtered, and the filter cake was washed with water (10 mL) to give 3-(4-bromophenyl)-4,6-difluoro-5-methoxy-1-methyl-1H-benzo[d]imidazol-2(3H)-one (74 mg, 95%) as an off-white solid. ¹H NMR (400 MHz, DMSO-d₆): δ 7.73 (d, J=8.4 Hz, 2H), 7.48 (br d, J=7.5 Hz, 2H), 7.29 (d, J=10.3 Hz, 1H), 3.83 (s, 3H), 3.36 (s, 3H); LCMS: 368.9 [M+H]⁺.

The Intermediate below was synthesized from Intermediate 18 and 2-iodopropane following the procedure described for Intermediate 20.

| Cmpd | Structure | Name | [M + H]⁺ |
|---|---|---|---|
| 20.1 | 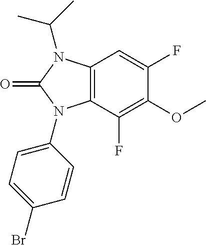 [SMILES: COc1c(F)cc2c(c1F)n(-c1ccc(Br)cc1)c(=O)n2C(C)C] | 3-(4-Bromophenyl)-4,6-difluoro- 1-isopropyl-5-methoxy-1H- benzo[d]imidazol-2(3H)-one | 396.9 |

Alternate conditions: 24 h.

### Intermediate 21

### 3-(4-Bromophenyl)-4,6-difluoro-5-methoxy-1-(methoxymethyl)-1H-benzo[d]imidazol-2(3H)-one

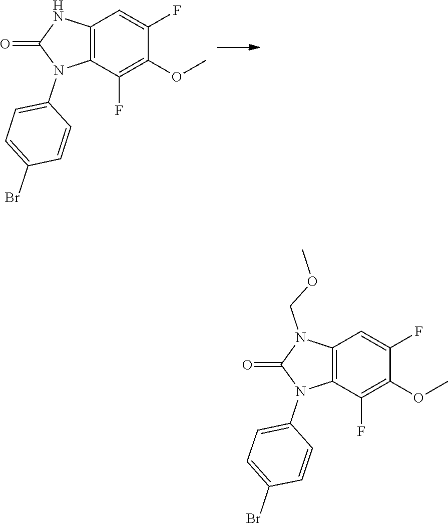 [SMILES: COc1c(F)cc2[nH]c(=O)n(-c3ccc(Br)cc3)c2c1F ; COCn1c(=O)n(-c2ccc(Br)cc2)c2c(F)c(OC)c(F)cc21]

Intermediate 18 (131 mg, 0.37 mmol) in DMF (1 mL) was added dropwise to a suspension of NaH (60% in mineral oil, 20 mg, 0.50 mmol) in DMF (0.5 mL). The reaction mixture was stirred at rt for 30 min. Chloromethyl methyl ether (31 μL, 0.41 mmol) in DMF (0.5 mL) was added dropwise. The reaction was stirred at rt for 2 h, poured into water (10 mL), and then extracted with CH₂Cl₂ (3×10 mL). The combined organic layers were dried (Na₂SO₄), filtered, concentrated, and then purified by silica gel chromatography (0-15% EtOAc in CH₂Cl₂) to give 3-(4-bromophenyl)-4,6-difluoro-5-methoxy-1-(methoxymethyl)-1H-benzo[d]imidazol-2(3H)-one (116 mg, 64%) as a white solid. ¹H NMR (400 MHz, DMSO-d₆): δ 7.74 (d, J=8.6 Hz, 2H), 7.52 (br d, J=7.2 Hz, 2H), 7.33 (d, J=10.1 Hz, 1H), 5.27 (s, 2H), 3.84 (s, 3H), 3.31 (s, 3H); LCMS: 354.9 [(M-CH₂OCH₃+H)+H]⁺.

### Intermediate 22

### 1-(4-Bromophenyl)-5,7-difluoro-1H-indazol-6-ol

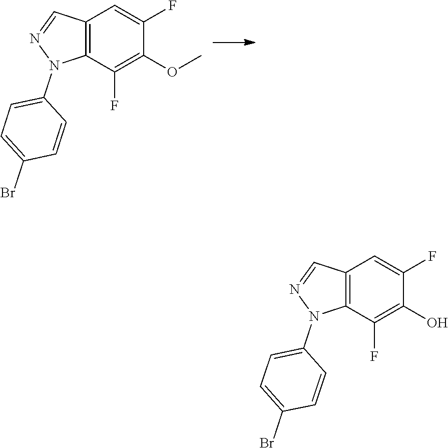 [SMILES: COc1c(F)cc2cnn(-c3ccc(Br)cc3)c2c1F ; Oc1c(F)cc2cnn(-c3ccc(Br)cc3)c2c1F]

Intermediate 14 (482 mg, 1.42 mmol) and pyridine hydrochloride (1.20 g, 10.4 mmol) were combined in an 8 mL vial. The reaction was stirred at 180° C. in a heating block for 4 h, cooled to 100° C., diluted with 5 mL 1N HCl, and then poured into a separatory funnel with 5 mL 1N HCl and 20 mL ethyl acetate. The layers were separated. The organics were washed with 20 mL brine, dried (Na₂SO₄), filtered, and then concentrated. The crude product was diluted with 5 mL CH₂Cl₂, sonicated, and then diluted with 15 mL hexanes. The solids were filtered off. The filter cake was washed with hexanes, dried on house vacuum, and then dried further on high vacuum to give 1-(4-bromophenyl)-5,7-difluoro-1H-indazol-6-ol (429 mg, 93%) as an off-white solid. ¹HNMR (400 MHz, DMSO-d₆): δ 10.64 (s, 1H), 8.32 (d, J=2.3 Hz, 1H), 7.74 (d, J=8.8 Hz, 2H), 7.62-7.51 (m, 3H); LCMS: 324.7 [M+H]⁺.

The Intermediates below were synthesized from the appropriate Intermediate following the procedure described for Intermediate 22.

| Cmpd | Structure | Name | [M + H]⁺ |
|---|---|---|---|
| 22.1 | 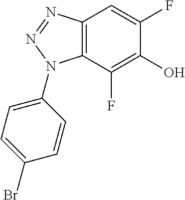 [SMILES: Oc1c(F)cc2nnn(-c3ccc(Br)cc3)c2c1F] | 1-(4-Bromophenyl)-5,7-difluoro- 1H-benzo[d][1,2,3]triazol-6-ol | 325.8 |
| 22.2 | 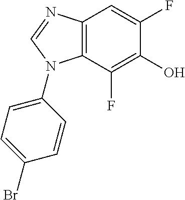 [SMILES: Oc1c(F)cc2ncn(-c3ccc(Br)cc3)c2c1F] | 1-(4-Bromophenyl)-5,7-difluoro- 1H-benzo[d]imidazol-6-ol | 324.8 |

Alternate conditions: 150-180° C., 1.5-4 h.

### Intermediate 23

### 1-(4-Bromophenyl)-5-fluoro-1H-indazol-6-ol

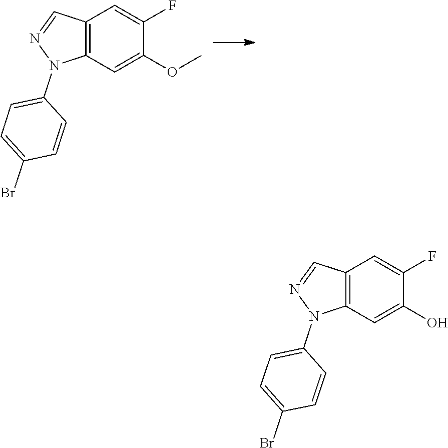 [SMILES: COc1cc2c(cnn2-c2ccc(Br)cc2)cc1F ; Oc1cc2c(cnn2-c2ccc(Br)cc2)cc1F]

A mixture of Intermediate 13.6 (340 mg, 1.06 mmol) and CH₂Cl₂ (3 mL) was cooled in a dry ice/acetone bath. Boron tribromide (2.1 mL, 2.1 mmol, 1.0 M in CH₂Cl₂) was added. The reaction was stirred at −78° C. for 6 h and stirred at 35° C. for 16 h. Additional boron tribromide (2.1 mL, 2.1 mmol, 1.0 M in CH₂Cl₂) was added. The reaction was stirred at 35° C. for 23 h. Additional boron tribromide (2.1 mL, 2.1 mmol, 1.0 M in CH₂Cl₂) was added. The reaction was stirred at 35° C. for 6 h, cooled in ice/water bath, quenched with methanol (2 mL), and then concentrated. The crude product was combined with another reaction and diluted with 20 mL EtOAc:CH₂Cl₂ (1:1). The organics were washed with 10 mL water, dried (Na₂SO₄), filtered, and then concentrated. The material was suspended in CH₂Cl₂ (5 mL), sonicated for 2 min, and then diluted with hexanes (5 mL). The mixture was stirred at rt. The solids were filtered off, and the filter cake was washed with hexanes to give 1-(4-bromophenyl)-5-fluoro-1H-indazol-6-ol (266 mg, 77%) as a dark pink solid. ¹H NMR (400 MHz, DMSO-d₆) δ 10.48 (s, 1H), 8.22 (d, J=0.6 Hz, 1H), 7.82-7.75 (m, 2H), 7.71-7.67 (m, 2H), 7.64 (d, J=10.5 Hz, 1H), 7.28 (d, J=7.1 Hz, 1H); LCMS: 306.8 [M+H]⁺.

### Intermediate 24

### 1-(4-Bromophenyl)-5,7-difluoro-6-((tetrahydro-2H-pyran-2-yl)oxy)-1H-indazole

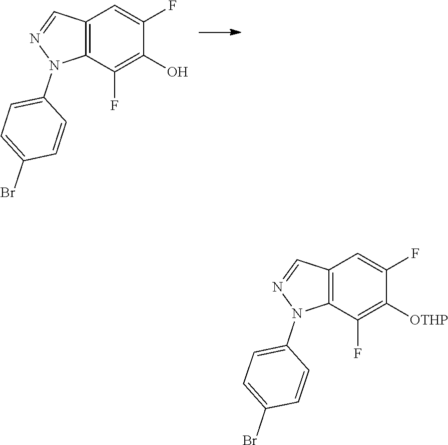

2H-3,4-Dihydropyran (0.37 mL, 4.06 mmol) was added to a mixture of Intermediate 22 (427 mg, 1.31 mmol), pyridinium p-toluenesulfonate (68 mg, 0.27 mmol), and CH₂Cl₂ (6 mL) at 0° C. The reaction was allowed to warm to rt, stirred for 16.5 h, and then diluted with 30 mL CH₂Cl₂. The organics were washed with 50 mL saturated NaHCO₃, dried (Na₂SO₄), filtered, concentrated, and then purified by silica gel chromatography (0-10% ethyl acetate in hexanes) to give 1-(4-bromophenyl)-5,7-difluoro-6-((tetrahydro-2H-pyran-2-yl)oxy)-1H-indazole (425 mg, 72%) as a clear thick oil. ¹H NMR (400 MHz, DMSO-d₆): δ 8.43 (d, J=2.2 Hz, 1H), 7.76 (d, J=8.7 Hz, 2H), 7.67 (d, J=9.8 Hz, 1H), 7.61 (dd, J=3.1, 8.7 Hz, 2H), 5.45 (s, 1H), 3.96 (td, J=7.0, 11.2 Hz, 1H), 3.60-3.52 (m, 1H), 1.96-1.80 (m, 3H), 1.69-1.54 (m, 3H); LCMS: 324.8 [(M-THP+H)+H]⁺.

### Intermediate 25

### 6-(Benzyloxy)-1-(5-bromopyridin-2-yl)-5,7-difluoro-1H-indazole

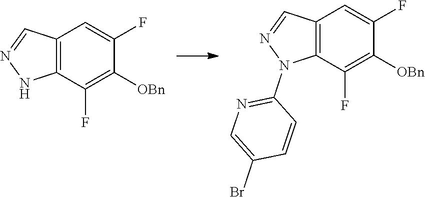 [SMILES: Fc1cc2cn[nH]c2c(F)c1OCc1ccccc1 ; Fc1cc2cnn(-c3ccc(Br)cn3)c2c(F)c1OCc1ccccc1]

A mixture of Intermediate 5 (500 mg, 1.92 mmol), Cs₂CO₃ (1.25 g, 3.84 mmol), 5-bromo-2-fluoropyridine (338 mg, 1.92 mmol), and DMF (10 mL) was stirred at 100° C. overnight, allowed to cool to rt, poured into water (30 mL), and then extracted with EtOAc (3×10 mL). The combined organic layers were washed with brine (10 mL), dried over Na₂SO₄, filtered, concentrated, and then purified by silica gel chromatography (petroleum ether/EtOAc=95/5) to give a ˜1:2 mixture of 6-(benzyloxy)-1-(5-bromopyridin-2-yl)-5,7-difluoro-1H-indazole and 6-(benzyloxy)-2-(5-bromopyridin-2-yl)-5,7-difluoro-2H-indazole (500 mg) as a yellow solid.

### 6-(benzyloxy)-1-(5-bromopyridin-2-yl)-5,7-difluoro-1H-indazole

¹H NMR (400 MHz, DMSO-d₆): δ 8.70 (d, 1H), 8.47 (s, 1H), 8.27 (dd, 1H), 7.79 (d, 1H), 7.67 (d, 1H), 7.50-7.45 (m, 2H), 7.43-7.30 (m, 3H), 5.23 (s, 2H); LCMS: 415.9 [M+H]⁺.

### 6-(benzyloxy)-2-(5-bromopyridin-2-yl)-5,7-difluoro-2H-indazole

¹H NMR (400 MHz, DMSO-d₆): δ 9.25 (d, 1H), 8.75 (d, 1H), 8.33 (dd, 1H), 8.16 (d, 1H), 7.53-7.32 (m, 6H), 5.28 (s, 2H); LCMS: 415.9 [M+H]⁺.

The Intermediate below was synthesized from Intermediate 5 and 5-bromo-2-fluoropyrimidine following the procedure described for Intermediate 25.

| Cmpd | Structure | Name | [M + H]⁺ |
|---|---|---|---|
| 25.1 | 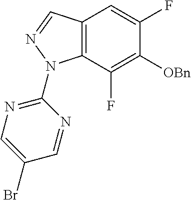 [SMILES: Fc1cc2cnn(-c3ncc(Br)cn3)c2c(F)c1OCc1ccccc1] | 6-(Benzyloxy)-1-(5- bromopyrimidin-2-yl)-5,7- difluoro-1H-indazole | 416.9 |

Alternate conditions: 70° C., 3 h.

### Intermediate 26

### 6-(Benzyloxy)-1-(5-bromopyrazin-2-yl)-5,7-difluoro-1H-indazole

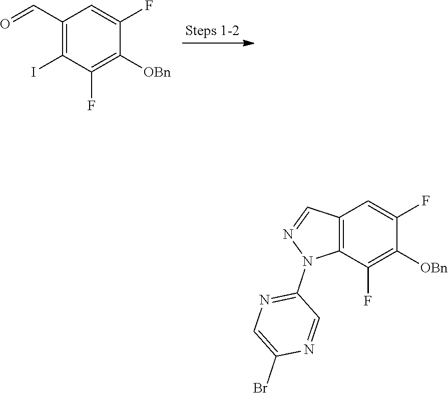 [SMILES: O=Cc1cc(F)c(OCc2ccccc2)c(F)c1I ; Fc1cc2cnn(-c3cnc(Br)cn3)c2c(F)c1OCc1ccccc1]

### Step 1: (Z)-2-(2-(4-(Benzyloxy)-3,5-difluoro-2-iodobenzylidene)hydrazinyl)-5-bromopyrazine

2-Bromo-5-hydrazinylpyrazine (505 mg, 2.67 mmol) was added to a solution of Intermediate 4 (1.00 g, 2.67 mmol) in EtOH (20 mL). The mixture was refluxed overnight, cooled to rt, and then filtered. The filter cake was washed with cold EtOH (10 mL) and then dried under reduced pressure to give (Z)-2-(2-(4-(benzyloxy)-3,5-difluoro-2-iodobenzylidene)hydrazinyl)-5-bromopyrazine (930 mg) as a white solid. ¹H NMR (400 MHz, DMSO-d₆): δ 11.69 (s, 1H), 8.55 (d, 1H), 8.34-8.25 (m, 2H), 7.80 (d, 1H), 7.48-7.32 (m, 5H), 5.22 (s, 2H); LCMS: 545.0 [M+H]⁺.

### Step 2: 6-(Benzyloxy)-1-(5-bromopyrazin-2-yl)-5,7-difluoro-1H-indazole

Copper(I) oxide (122 mg, 0.853 mmol) was added to a solution of (Z)-2-(2-(4-(benzyloxy)-3,5-difluoro-2-iodobenzylidene)hydrazinyl)-5-bromopyrazine (930 mg, 1.71 mmol) in 3-methylbutan-1-ol (15 mL) under N₂. The mixture was degassed with 3 vacuum/N₂ cycles, refluxed overnight, cooled to rt, poured into water (10 mL), and then extracted with EtOAc (3×10 mL). The combined organic layers were washed with brine (20 mL), dried over Na₂SO₄, filtered, concentrated, and then purified by silica gel chromatography (petroleum ether/ethyl acetate=30/1 to 5/1) to give 6-(benzyloxy)-1-(5-bromopyrazin-2-yl)-5,7-difluoro-1H-indazole (520 mg, 46% over two steps) as a white solid. ¹H NMR (400 MHz, DMSO-d₆): δ 8.99 (d, 1H), 8.87 (d, 1H), 8.59-8.55 (m, 1H), 7.76-7.67 (m, 1H), 7.50-7.44 (m, 2H), 7.43-7.33 (m, 3H), 5.24 (s, 2H); LCMS: 417.1 [M+H]⁺.

The Intermediate below was synthesized from Intermediate 4.1 following the procedures described for Intermediate 26.

| Cmpd | Structure | Name | [M + H]⁺ |
|---|---|---|---|
| 26.1 | 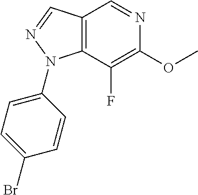 [SMILES: COc1ncc2cnn(-c3ccc(Br)cc3)c2c1F] | 1-(4-Bromophenyl)-7-fluoro-6- methoxy-1H-pyrazolo[4,3- c]pyridine | 322.0 |

### Intermediate 27

### 1-(4-Bromophenyl)-6-(tert-butoxy)-5-fluoro-1H-pyrazolo[3,4-b]pyridine

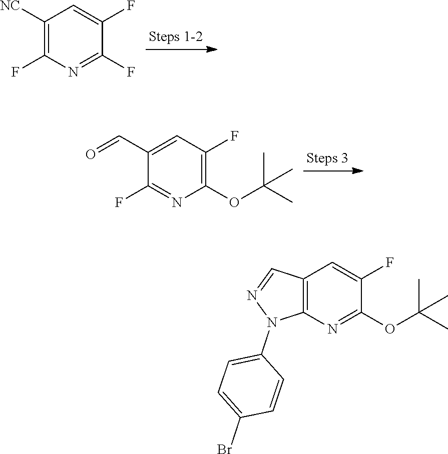 [SMILES: N#Cc1cc(F)c(F)nc1F ; CC(C)(C)Oc1nc(F)c(C=O)cc1F ; CC(C)(C)Oc1nc2c(cnn2-c2ccc(Br)cc2)cc1F]

### Step 1: 6-(tert-Butoxy)-2,5-difluoronicotinonitrile

A solution of t-BuOK (7.95 g, 70.85 mmol), t-BuOH (69 mL), and THF (13 mL) was added to a mixture of 2,5,6-trifluoronicotinonitrile (10 g, 63 mmol), DMSO (2.9 mL), t-BuOH (50 mL), and THF (10 mL) at 0° C. The mixture was allowed to warm to rt, stirred for 0.5 h, and then poured into 30 mL saturated NH₄Cl. The mixture was extracted with EtOAc (3×30 mL). The combined organic layers were washed with brine (2×50 mL), dried over Na₂SO₄, filtered, concentrated, and then purified by silica gel chromatography (petroleum ether/ethyl acetate=20/1 to 1/1) to give 6-(tert-butoxy)-2,5-difluoronicotinonitrile (10 g, 74%) as a white oil. ¹H NMR (400 MHz, DMSO-d₆): δ7.56 (d, 1H), 1.65 (s, 9H); LCMS: 213.1 [M+H]⁺.

### Step 2: 6-(tert-Butoxy)-2,5-difluoronicotinaldehyde

Diisobutylaluminum hydride (1 M in toluene, 94 mL, 94 mmol) was added to a solution of 6-(tert-butoxy)-2,5-difluoronicotinonitrile (10 g, 47 mmol) in toluene (200 mL) at −78° C. The mixture was allowed to warm to rt, stirred for 1 h, and then poured into saturated NaK tartrate (200 mL). The mixture was stirred for 1 h and then filtered through Celite. The filtrate was extracted with EtOAc (3×100 mL). The combined organic layers were washed with brine (2×100 mL), dried over Na₂SO₄, filtered, concentrated, and then purified by silica gel chromatography (petroleum ether/ethyl acetate=50/1 to 10/1) to give 6-(tert-butoxy)-2,5-difluoronicotinaldehyde (6 g, 59%) as a white solid. ¹H NMR (400 MHz, DMSO-d₆): δ 9.98 (s, 1H), 8.07 (d, 1H), 1.61 (s, 9H).

### Step 3: 1-(4-Bromophenyl)-6-(tert-butoxy)-5-fluoro-LH-pyrazolo[3,4-b]pyridine

A mixture of 6-(tert-butoxy)-2,5-difluoronicotinaldehyde (2.00 g, 9.29 mmol), (4-bromophenyl)hydrazine (2.08 g, 9.29 mmol, HCl salt), and NMP (30 mL) was stirred at rt for 2 h. Cesium carbonate (9.08 g, 27.88 mmol) was added. The mixture was stirred at 115° C. for 1 h, cooled to rt, poured into H₂O (30 mL), and then extracted with EtOAc (3×30 mL). The combined organic layers were washed with brine (2×50 mL), dried over Na₂SO₄, filtered, concentrated, and then purified by silica gel chromatography (petroleum ether/ethyl acetate=20/1 to 5/1) to give 1-(4-bromophenyl)-6-(tert-butoxy)-5-fluoro-1H-pyrazolo[3,4-b]pyridine (2.60 g, 76%) as a yellow solid. ¹H NMR (400 MHz, DMSO-d₆): δ 8.25 (s, 1H), 8.11 (d, 1H), 8.09-8.05 (m, 2H), 7.81-7.69 (m, 2H), 1.66 (s, 9H); LCMS: 364.0 [M+H]⁺.

### Intermediate 28

### (3′-Methoxy-[1,1′-biphenyl]-4-yl)hydrazine

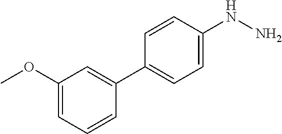 [SMILES: COc1cccc(-c2ccc(NN)cc2)c1]

3′-Methoxy-[1,1′-biphenyl]-4-amine (3.00 g, 15.06 mmol) was added to aqueous HCl (37%, 30 mL) at 0° C. The mixture was warmed to rt, stirred for 20 min, and then cooled to 0° C. A solution of NaNO₂ (1.14 g, 16.56 mmol) in H₂O (15 mL) was added dropwise keeping the internal temperature at ˜0° C. A solution of SnCl₂·2H₂O (13.59 g, 60.23 mmol) in aqueous HCl (37%, 60 mL) was added dropwise (internal temperature remained ˜0° C.). The mixture was warmed to rt, stirred for 3 h, and then filtered. The filter cake was washed with aqueous HCl (37%, 30 mL) and then dried under vacuum. The residue was diluted with saturated NaHCO₃ until pH=7, stirred for 30 min, and then extracted with ethyl acetate (3×40 mL). The combined organic layers were dried with Na₂SO₄, filtered, concentrated, and then triturated with PE:EA (15 mL, 10:1) to give (3′-methoxy-[1,1′-biphenyl]-4-yl)hydrazine (1.80 g) as a yellow solid. ¹H NMR (400 MHz, DMSO-d₆): δ 7.43 (d, 2H), 7.29-7.25 (m, 1H), 7.14-7.11 (m, 1H), 7.09-7.07 (m, 1H), 6.87-6.81 (m, 3H), 6.80-6.75 (m, 1H), 4.01 (s, 2H), 3.79 (s, 3H); LCMS: 215.4 [M+H]⁺.

The Intermediate below was synthesized from Intermediate 32 following the procedure described for Intermediate 28.

| Int | Structure | Name | [M + H]⁺ |
|---|---|---|---|
| 28.1 | 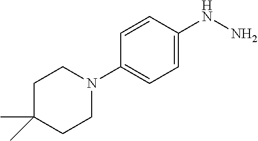 [SMILES: CC1(C)CCN(c2ccc(NN)cc2)CC1] | 1-(4-Hydrazinylphenyl)-4,4- dimethylpiperidine hydrochloride | 220.2 |

Alternate conditions: After washing filter cake with HCl and drying, the material was triturated in EtOAc/CH₃OH (4/1, 100 mL), rt, 2 h and then filtered. The filter cake was washed with EtOAc (10 mL) and then dried to give the HCl salt.

### Intermediate 29

### 1-(4-Bromophenyl)-4,4-dimethylpiperidine

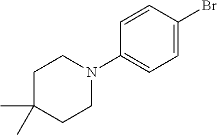

A mixture of 1-bromo-4-iodobenzene (1.87 g, 6.62 mmol), 4,4-dimethylpiperdine hydrochloride (500 mg, 3.34 mmol), tris(dibenzylideneacetone)dipalladium (101 mg, 0.11 mmol), RuPhos (51 mg, 0.11 mmol), sodium tert-butoxide (1.27 g, 13.2 mmol), and toluene (8 mL) was degassed with 2 vacuum/N₂ cycles, stirred at 100° C. for 1 h, allowed to cool to rt, diluted with EtOAc (10 mL) and water (10 mL), and then filtered through Celite. The filter cake was washed with EtOAc (5 mL) and water (5 mL). The organic layer was separated, washed with brine (10 mL), dried (Na₂SO₄), filtered, concentrated, and then purified by silica gel chromatography (0-30% CH₂Cl₂ in hexanes then 0-15% EtOAc in hexanes) to give 1-(4-bromophenyl)-4,4-dimethylpiperidine (269 mg, 30%) as a beige solid. ¹H NMR (400 MHz, DMSO-d₆) δ 7.27-7.20 (m, 2H), 6.85-6.78 (m, 2H), 3.11-3.02 (m, 4H), 1.39-1.29 (m, 4H), 0.87 (s, 6H); LCMS: 267.8 [M+H]⁺.

### Intermediate 30

### (2-(4-(Methylsulfonyl)piperazin-1-yl)pyrimidin-5-yl)boronic Acid

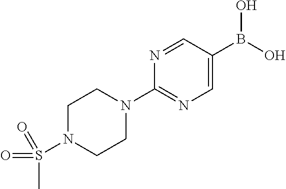 [SMILES: CS(=O)(=O)N1CCN(c2ncc(B(O)O)cn2)CC1]

A mixture of (2-chloropyrimidin-5-yl)boronic acid (1 g, 6.32 mmol), 1-(methylsulfonyl)piperazine (1.03 g, 6.32 mmol), Et₃N (1.60 g, 15.79 mmol), and EtOH (10 mL) was stirred at 80° C. for 3 h, allowed to cool to rt, poured into water (20 mL), and then extracted with EtOAc (3×10 mL). The combined organic layers were washed with brine (10 mL), dried over Na₂SO₄, filtered, concentrated, and then purified by silica gel chromatography (CH₂Cl₂/CH₃OH=1/1) to give (2-(4-(methylsulfonyl)piperazin-1-yl)pyrimidin-5-yl)boronic acid (600 mg, 33%) as a yellow solid. ¹H NMR (400 MHz, DMSO-d₆): δ 8.65 (s, 2H), 8.11 (s, 2H), 3.94-3.83 (m, 4H), 3.21-3.11 (m, 4H), 2.88 (s, 3H); LCMS: 286.9 [M+H]⁺.

The Intermediate below was synthesized from 2-chloro-5-iodopyrimidine and 4,4-dimethylpiperidine hydrochloride following the procedure described for Intermediate 30.

| Cmpd | Structure | Name | [M + H]⁺ |
|---|---|---|---|
| 30.1 | 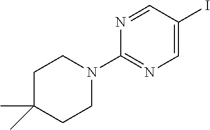 | 2-(4,4-Dimethylpiperidin-1-yl)-5- iodopyrimidine | 318.0 |
| 30.2 | 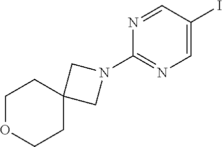 [SMILES: Ic1cnc(N2CC3(CCOCC3)C2)nc1] | 2-(5-Iodopyrimidin-2-yl)-7-oxa-2- azaspiro[3.5]nonane | 332.0 |

Alternate conditions: i-Pr₂NEt, EtOH, 70° C., 3 h.

### Intermediate 31

### (4-(4,4-Dimethylpiperidin-1-yl)phenyl)boronic Acid

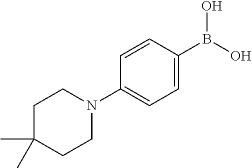 [SMILES: CC1(C)CCN(c2ccc(B(O)O)cc2)CC1]

n-Butyllithium (2.5 M in hexanes, 1.43 mL) was added to a solution of 1-(4-bromophenyl)-4,4-dimethylpiperidine (800 mg, 2.98 mmol) in THF (10 mL) at −78° C. under N₂. The mixture was stirred for 2 h. Trimethyl borate (433.9 mg, 4.18 mmol) was added. The mixture was allowed to warm to rt, stirred for 10 h, poured into sat. aq. NH₄Cl (10 mL), and then extracted with EtOAc (3×10 mL). The combined organic layers were washed with brine (2×10 mL), dried over Na₂SO₄, filtered, and then concentrated. The residue was triturated in in PE:EtOAc=5:1 (10 mL) at rt for 30 min, filtered, and then dried to give (4-(4,4-dimethylpiperidin-1-yl)phenyl)boronic acid (580 mg) as a white solid. ¹H NMR (400 MHz, DMSO-d₆): δ 7.64-7.62 (m, 4H), 6.87-6.85 (m, 2H), 3.21-3.18 (m, 4H), 1.43-1.40 (m, 4H), 0.95 (s, 6H); LCMS: 234.2 [M+H]⁺.

### Intermediate 32

### 4-(4,4-Dimethylpiperidin-1-yl)aniline

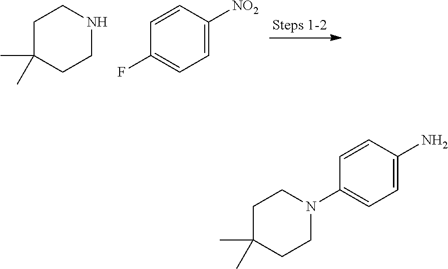

### Step 1: 4,4-Dimethyl-1-(4-nitrophenyl)piperidine

A mixture of 1-fluoro-4-nitrobenzene (9.06 g, 64.2 mmol), 4,4-dimethylpiperidine (8 g, 53 mmol, HCl), K₂CO₃ (26.6 g, 193 mmol), and DMF (100 mL) was stirred at 70° C. overnight, allowed to cool to rt, poured into water (200 ml), and then extracted with MTBE (3×200 mL). The combined organics were washed with brine (200 mL), dried over Na₂SO₄, filtered, concentrated, and then purified by silica gel chromatography (petroleum ether/EtOAc=95/5) to give 4,4-dimethyl-1-(4-nitrophenyl)piperidine (10 g, 66%) as a yellow solid. ¹H NMR (400 MHz, DMSO-d₆): δ 8.01 (d, 2H), 6.98 (d, 2H), 3.56-3.38 (m, 4H), 1.46-1.30 (m, 4H), 0.97 (s, 6H); LCMS: 235.2 [M+H]⁺.

### Step 2: 4-(4,4-Dimethylpiperidin-1-yl)aniline

4,4-Dimethyl-1-(4-nitrophenyl)piperidine (10 g, 42.7 mmol) was added to a mixture of Pd/C (10%, 2 g) in CH₃OH (150 mL) under N₂. The suspension was degassed under vacuum and purged with H₂ several times. The mixture was stirred under H₂ (15 psi) at rt for 2 h and then filtered through Celite. The filter cake was washed with CH₃OH (3×100 mL). The combined organic layers were concentrated and then purified by silica gel chromatography (petroleum ether/EtOAc=80/20) to give 4-(4,4-dimethylpiperidin-1-yl)aniline (7 g, 80%) as a black brown solid. ¹H NMR (400 MHz, DMSO-d₆): δ 6.69 (d, 2H), 6.46 (d, 2H), 4.52 (s, 2H), 2.93-2.80 (m, 4H), 1.47-1.37 (m, 4H), 0.93 (s, 6H); LCMS: 205.1 [M+H]⁺.

### Intermediate 33

### 6-(Benzyloxy)-5-fluoro-1H-indazole-7-carbonitrile

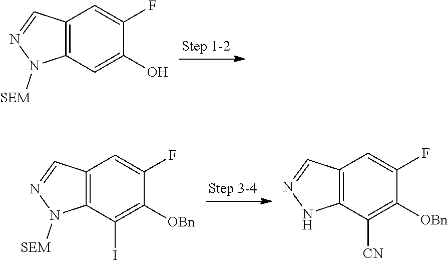 [SMILES: C[Si](C)(C)CCOCn1ncc2cc(F)c(O)cc21 ; C[Si](C)(C)CCOCn1ncc2cc(F)c(OCc3ccccc3)c(I)c21 ; N#Cc1c(OCc2ccccc2)c(F)cc2cn[nH]c12]

### Step 1: 5-Fluoro-7-iodo-1-((2-(trimethylsilyl)ethoxy)methyl)-1H-indazol-6-ol

N-Iodosuccinimide (4.38 g, 19.5 mmol) was added to a solution of Intermediate 9, Step 2 (5.00 g, 17.7 mmol) in THF (50 mL). The mixture was stirred at rt for 1 h, concentrated, and then purified by column chromatography (petroleum ether/ethyl acetate=50/1 to 5/1) to give 5-fluoro-7-iodo-1-((2-(trimethylsilyl)ethoxy)methyl)-1H-indazol-6-ol (1.02 g, 14%) as a yellow solid. ¹H NMR (400 MHz, DMSO-d₆): δ 10.71 (s, 1H), 8.06 (s, 1H), 7.60 (d, 1H), 5.98 (s, 2H), 3.49 (t, 2H), 0.77 (t, 2H), −0.11 (s, 9H); LCMS: 409.1 [M+H]⁺.

### Step 2: 6-(Benzyloxy)-5-fluoro-7-iodo-1-((2-(trimethylsilyl)ethoxy)methyl)-1H-indazole

Potassium carbonate (691 mg, 5.00 mmol) was added to a solution of 5-fluoro-7-iodo-1-((2-(trimethylsilyl)ethoxy)methyl)-1H-indazol-6-ol (1.02 g, 2.50 mmol) in DMF (10 mL). The suspension was stirred at rt for 1 h. Benzyl bromide (641 mg, 3.75 mmol) was added. The mixture was stirred at rt overnight, poured into H₂O (10 mL), and then extracted with EtOAc (3×15 mL). The combined organic layers were washed with brine (2×10 mL), dried over Na₂SO₄, filtered, concentrated, and then purified by column chromatography (petroleum ether/ethyl acetate=20/1 to 4/1) to give 6-(benzyloxy)-5-fluoro-7-iodo-1-((2-(trimethylsilyl)ethoxy)methyl)-1H-indazole (1.10 g, 88%) as a yellow solid. ¹H NMR (400 MHz, DMSO-d₆): δ 8.14 (s, 1H), 7.76 (d, 1H), 7.59-7.57 (m, 2H), 7.45-7.38 (m, 3H), 6.02 (s, 2H), 5.06 (s, 2H), 3.51 (t, 2H), 0.78 (t, 2H), −0.11 (s, 9H); LCMS: 499.1 [M+H]⁺.

### Step 3: 6-(Benzyloxy)-5-fluoro-1-((2-(trimethylsilyl)ethoxy)methyl)-1H-indazole-7-carbonitrile

Copper(I) cyanide (395 mg, 4.41 mmol) was added to a solution of 6-(benzyloxy)-5-fluoro-7-iodo-1-((2-(trimethylsilyl)ethoxy)methyl)-1H-indazole (1.10 g, 2.21 mmol) in DMF (10 mL). The mixture was stirred at 140° C. overnight, allowed to cool to rt, poured into H₂O (20 mL), and then extracted with EtOAc (3×10 mL). The combined organic layers were washed with brine (2×10 mL), dried over Na₂SO₄, filtered, concentrated, and then purified by column chromatography (petroleum ether/ethyl acetate=20/1 to 3/1) to give 6-(benzyloxy)-5-fluoro-1-((2-(trimethylsilyl)ethoxy)methyl)-1H-indazole-7-carbonitrile (570 mg, 64%) as a yellow solid. ¹H NMR (400 MHz, DMSO-d₆): δ 8.26 (s, 1H), 8.16 (d, 1H), 7.51-7.49 (m, 2H), 7.43-7.37 (m, 3H), 5.84 (s, 2H), 5.38 (s, 2H), 3.51 (t, 2H), 0.79 (t, 2H), −0.12 (s, 9H).

### Step 4: 6-(Benzyloxy)-5-fluoro-1H-indazole-7-carbonitrile

Trifluoroacetic acid (3.2 mL, 42.8 mmol) was added to a solution of 6-(benzyloxy)-5-fluoro-1-((2-(trimethylsilyl)ethoxy)methyl)-1H-indazole-7-carbonitrile (570 mg, 1.43 mmol) in DCE (3 mL). The mixture was stirred at rt for 3 h, concentrated, dissolved into EtOAc (10 mL), and then diluted with sat. aq. NaHCO₃ (˜10 mL) until pH=˜7. The layers were separated. The aqueous layer was extracted with EtOAc (3×10 mL). The combined organic layers were washed with brine (10 mL), dried over Na₂SO₄, filtered, concentrated, and then purified by column chromatography (petroleum ether/ethyl acetate=10/1 to 1/1) to 6-(benzyloxy)-5-fluoro-1H-indazole-7-carbonitrile (260 mg, 67%) as a white solid. ¹HNMR (400 MHz, DMSO-d₆): δ 13.95 (s, 1H), 8.20 (s, 1H), 8.10 (d, 1H), 7.50-7.48 (m, 2H), 7.43-7.36 (m, 3H), 5.38 (s, 2H); LCMS: 268.1 [M+H]⁺.

### Intermediate 34

### 5-Fluoro-6-methoxy-1H-indazole-4-carbonitrile

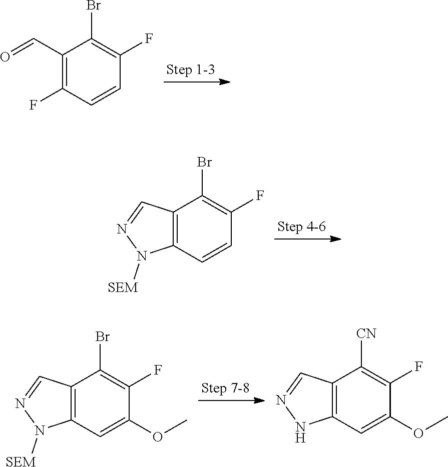 [SMILES: O=Cc1c(F)ccc(F)c1Br ; C[Si](C)(C)CCOCn1ncc2c(Br)c(F)ccc21 ; COc1cc2c(cnn2COCC[Si](C)(C)C)c(Br)c1F ; COc1cc2[nH]ncc2c(C#N)c1F]

### Step 1: 2-Bromo-3,6-difluorobenzaldehyde O-methyl oxime

Potassium carbonate (51.8 g, 375 mmol) was added to a solution of 2-bromo-3,6-difluoro-benzaldehyde (55.2 g, 250 mmol), O-methylhydroxylamine HCl (22.9 g, 275 mmol), and DME (1000 mL). The mixture was stirred at 60° C. for 4 h, allowed to cool to rt, poured into H₂O (500 mL), and then extracted with EtOAc (3×1000 mL). The combined organic layers were washed with brine (2×1000 mL), dried over Na₂SO₄, filtered, concentrated, and then purified by column chromatography (petroleum ether/ethyl acetate=50/1 to 20/1) to give 2-bromo-3,6-difluorobenzaldehyde O-methyl oxime (60.4 g, 96%) as a yellow solid. ¹H NMR (400 MHz, DMSO-d₆): δ 8.25-7.65 (m, 1H), 7.55-7.49 (m, 1H), 7.46-7.40 (m, 1H), 3.94-3.85 (m, 3H).

### Step 2: 4-Bromo-5-fluoro-1H-indazole

Hydrazine monohydrate (310 mL, 6.25 mol) was added to a solution of 2-bromo-3,6-difluorobenzaldehyde O-methyl oxime (60.4 g, 242 mmol) in NMP (600 mL). The mixture was stirred at 130° C. overnight, allowed to cool to rt, poured into H₂O (200 mL), and then filtered. The filter cake was washed with H₂O (100 mL) and dried under vacuum to give 4-bromo-5-fluoro-1H-indazole (42 g) as a white solid. ¹H NMR (400 MHz, DMSO-d₆): δ 13.53 (s, 1H), 8.07 (s, 1H), 7.60 (d, 1H), 7.38 (t, 1H); LCMS: 215.0 [M+H]⁺.

### Step 3: 4-Bromo-5-fluoro-1-((2-(trimethylsilyl)ethoxy)methyl)-1H-indazole

Sodium hydride (60%, 11.9 g, 298 mmol) was added to a solution of 4-bromo-5-fluoro-1H-indazole (32.0 g, 149 mmol) in DMF (350 mL) at 0° C. The mixture was stirred for 1 h. SEM-Cl (49.6 g, 298 mmol) was added at 0° C. The reaction was allowed to warm to rt overnight, poured into H₂O (300 mL), and then extracted with EtOAc (3×300 mL). The combined organic layers were washed with brine (2×300 mL), dried over Na₂SO₄, filtered, concentrated, and then purified by column chromatography (petroleum ether/ethyl acetate=50/1 to 20/1) to give 4-bromo-5-fluoro-1-((2-(trimethylsilyl)ethoxy)methyl)-1H-indazole (22 g, 34%) as a yellow oil. ¹H NMR (400 MHz, DMSO-d₆): δ 8.16 (s, 1H), 7.85 (d, 1H), 7.50 (t, 1H), 5.78 (s, 2H), 3.50 (t, 2H), 0.79 (t, 2H), −0.11 (s, 9H); LCMS: 345.1 [M+H]⁺.

### Step 4: 4-Bromo-5-fluoro-6-(4,4,5,5-tetramethyl-1,3,2-dioxaborolan-2-yl)-1-((2-(trimethylsilyl)ethoxy)methyl)-1H-indazole

Bis(1,5-cyclooctadiene)dimethoxydiiridium (192 mg, 0.289 mmol) was added to a solution of 4-bromo-5-fluoro-1-((2-(trimethylsilyl)ethoxy)methyl)-1H-indazole (5.00 g, 14.5 mmol), 4,4,4′,4′,5,5,5′,5′-octamethyl-2,2′-bi(1,3,2-dioxaborolane) (7.35 g, 28.9 mmol), 4,4′-di-tert-butyl-2,2′-bipyridine (194 mg, 0.724 mmol), and THF (100 mL) under N₂. The mixture was degassed and purged with N₂ 3 times, stirred at 80° C. for 1 h, allowed to cool to rt, concentrated, and then purified by column chromatography (petroleum ether/ethyl acetate=0/1 to 0/1) to give 4-bromo-5-fluoro-6-(4,4,5,5-tetramethyl-1,3,2-dioxaborolan-2-yl)-1-((2-(trimethylsilyl)ethoxy)methyl)-1H-indazole (7 g) as a yellow oil. LCMS: 471.2 [M+H]⁺.

### Step 5: 4-Bromo-5-fluoro-1-((2-(trimethylsilyl)ethoxy)methyl)-1H-indazol-6-ol

Sodium perborate tetrahydrate (9.14 g, 59.4 mmol) was added to a solution of 4-bromo-5-fluoro-6-(4,4,5,5-tetramethyl-1,3,2-dioxaborolan-2-yl)-1-((2-(trimethylsilyl)ethoxy)methyl)-1H-indazole (7.00 g, 14.9 mmol), THF (60 mL), and CH₃OH (20 mL). The mixture was stirred at rt overnight, poured into water (40 ml), and then extracted with EtOAc (2×60 ml). The combined organic layers were washed with brine (40 ml), dried over Na₂SO₄, filtered, concentrated, and then purified by column chromatography (petroleum ether/ethyl acetate=20/1 to 2/1) to give 4-bromo-5-fluoro-1-((2-(trimethylsilyl)ethoxy)methyl)-1H-indazol-6-ol (4 g, 74% yield) as a yellow oil. ¹H NMR (400 MHz, DMSO-d₆): δ 10.72 (s, 1H), 7.92 (s, 1H), 7.13 (d, 1H), 5.63 (s, 2H), 3.47 (t, 2H), 0.78 (t, 2H), −0.11 (s, 9H); LCMS: 361.1 [M+H]⁺.

### Step 6: 4-Bromo-5-fluoro-6-methoxy-1-((2-(trimethylsilyl)ethoxy)methyl)-1H-indazole

Potassium carbonate (1.78 g, 12.8 mmol) was added to a solution of 4-bromo-5-fluoro-1-((2-(trimethylsilyl)ethoxy)methyl)-1H-indazol-6-ol (4.00 g, 11.1 mmol) in DMF (50 mL). The mixture was stirred at rt for 1 h. Iodomethane (2.76 mL, 44.3 mmol) was added. The mixture was stirred at rt overnight, poured into H₂O (30 mL), and then extracted with EtOAc (3×30 mL). The combined organic layers were washed with brine (2×50 mL), dried over Na₂SO₄, filtered, concentrated, and then purified by column chromatography (petroleum ether/ethyl acetate=20/1 to 1/1) to give 4-bromo-5-fluoro-6-methoxy-1-((2-(trimethylsilyl)ethoxy)methyl)-1H-indazole (2.20 g, 52%) as a yellow oil. ¹H NMR (400 MHz, DMSO-d₆): δ 8.03 (s, 1H), 7.50 (d, 1H), 5.74 (s, 2H), 3.94 (s, 3H), 3.53 (t, 2H), 0.80 (t, 2H), −0.10 (s, 9H); LCMS: 375.1 [M+H]⁺.

### Step 7: 5-Fluoro-6-methoxy-1-((2-(trimethylsilyl)ethoxy)methyl)-1H-indazole-4-carbonitrile

Pd(PPh₃)₄ (262 mg, 0.226 mmol) was added to a solution of 4-bromo-5-fluoro-6-methoxy-1-((2-(trimethylsilyl)ethoxy)methyl)-1H-indazole (1.70 g, 4.53 mmol), Zn(CN)₂ (585 mg, 4.98 mmol), and DMF (20 mL) under N₂. The mixture was degassed and purged with N₂ 3 times, stirred at 145° C. for 0.5 h in a microwave, allowed to cool to rt, poured into H₂O (15 mL), and then extracted with EtOAc (3×15 mL). The combined organic layers were washed with brine (2×10 mL), dried over Na₂SO₄, filtered, concentrated, and then purified by column chromatography (petroleum ether/ethyl acetate=20/1→1/1) to give 5-fluoro-6-methoxy-1-((2-(trimethylsilyl)ethoxy)methyl)-1H-indazole-4-carbonitrile (1.10 g, 75%) as a white solid. ¹HNMR (400 MHz, DMSO-d₆): δ 8.28 (s, 1H), 7.92 (d, 1H), 5.80 (s, 2H), 3.99 (s, 3H), 3.54 (t, 2H), 0.80 (t, 2H), −0.10 (s, 9H); LCMS: 322.2[M+H]⁺.

### Step 8: 5-Fluoro-6-methoxy-1H-indazole-4-carbonitrile

Concentrated HCl (37%, 81.1 mL, 839 mmol) was added to a solution of 5-fluoro-6-methoxy-1-((2-(trimethylsilyl)ethoxy)methyl)-1H-indazole-4-carbonitrile (1.20 g, 3.73 mmol) in AcOH (30 mL). The mixture was stirred at rt overnight, concentrated, and then purified by column chromatography (petroleum ether/ethyl acetate=0/1 to 0/1) to give a mixture of 5-fluoro-6-methoxy-1H-indazole-4-carbonitrile and 5-fluoro-1-(hydroxymethyl)-6-methoxy-1H-indazole-4-carbonitrile (658 mg). The mixture was dissolved in EtOAc (30 mL). Ethane-1,2-diamine (10 mL, 187 mmol) was added. The mixture was stirred at rt overnight, poured into H₂O (20 mL), and then extracted with EtOAc (3×30 mL). The combined organic layers were washed with brine (2×50 mL), dried over Na₂SO₄, filtered, concentrated, and then purified by column chromatography (petroleum ether/ethyl acetate=3/1 to 0/1) to give 5-fluoro-6-methoxy-1H-indazole-4-carbonitrile (402 mg, 56%) as a white solid. ¹H NMR (400 MHz, DMSO-d₆): δ 13.56 (s, 1H), 8.18 (s, 1H), 7.56 (d, 1H), 3.97 (s, 3H); LCMS: 191.9 [M+H]⁺.

### Intermediate 35

### 6-(Benzyloxy)-5,7-difluoro-1H-indole

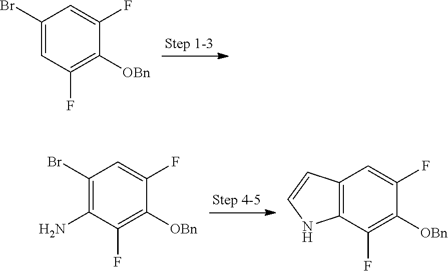 [SMILES: Fc1cc(Br)cc(F)c1OCc1ccccc1 ; Nc1c(Br)cc(F)c(OCc2ccccc2)c1F ; Fc1cc2cc[nH]c2c(F)c1OCc1ccccc1]

### Step 1: 3-(Benzyloxy)-6-bromo-2,4-difluorobenzoic Acid

LDA (2 M in THF, 63.0 mL, 126 mmol) was added to a solution of 2-(benzyloxy)-5-bromo-1,3-difluorobenzene (25.1 g, 83.9 mmol) in THF (300 mL) at −78° C. under N₂. The reaction mixture was stirred for 2 h. CO₂ (gas) was bubbled into the reaction mixture for 1 h. The mixture was allowed to warm to rt, stirred overnight, poured into sat. NH₄Cl (500 mL), and then extracted with EtOAc (3×200 mL). The combined organic layers were washed with brine (200 mL), dried over Na₂SO₄, filtered, concentrated, and then purified by silica gel chromatography (EtOAc/EtOH=3/1) to give 3-(benzyloxy)-6-bromo-2,4-difluorobenzoic acid (18.5 g, 64%) as a yellow solid. ¹HNMR (400 MHz, DMSO-d₆): δ 8.73 (s, 1H), 7.48-7.33 (m, 5H), 7.32-7.26 (m, 1H), 5.11 (s, 2H).

### Step 2: tert-Butyl (3-(benzyloxy)-6-bromo-2,4-difluoro phenyl)carbamate

DPPA (17.5 mL, 80.9 mmol) was added to a solution of 3-(benzyloxy)-6-bromo-2,4-difluorobenzoic acid (18.5 g, 53.9 mmol), Et₃N (8.18 g, 80.9 mmol), and t-BuOH (200 mL). The reaction mixture was stirred at 80° C. overnight, allowed to cool to rt, and then concentrated to give tert-butyl (3-(benzyloxy)-6-bromo-2,4-difluorophenyl)carbamate (20 g) as a yellow oil. LCMS: 411.9 [M−H]⁻.

### Step 3: 3-(Benzyloxy)-6-bromo-2,4-difluoroaniline

A mixture of tert-butyl (3-(benzyloxy)-6-bromo-2,4-difluorophenyl)carbamate (20 g, 48.3 mmol), CH₂Cl₂ (300 mL), and TFA (40 mL) was stirred at rt for 2 h, concentrated, and then dissolved in water (50 mL). The mixture was adjusted to pH˜8 with sat. aq. NaHCO₃ (100 mL) and then extracted with EtOAc (3×50 mL). The combined organic layers were washed with brine (50 mL), dried over Na₂SO₄, filtered, concentrated, and then purified by silica gel chromatography (petroleum ether/ethyl acetate=9/1) to give 3-(benzyloxy)-6-bromo-2,4-difluoroaniline (3 g, 18% over 2 steps) as a yellow oil. ¹H NMR (400 MHz, DMSO-d₆): δ 7.46-7.23 (m, 6H), 5.27-5.05 (m, 4H).

### Step 4: 3-(Benzyloxy)-2,4-difluoro-6-((trimethylsilyl)ethynyl)aniline

Copper(I) iodide (182 mg, 0.955 mmol) and Pd(dppf)Cl₂ (699 mg, 0.955 mmol) were added to a mixture of 3-(benzyloxy)-6-bromo-2,4-difluoroaniline (3 g, 9.55 mmol), ethynyltrimethylsilane (2.81 g, 28.7 mmol), and Et₃N (40 mL) under N₂. The reaction mixture was stirred at 90° C. overnight, allowed to cool to rt, and then filtered through Celite. The Celite pad was washed with EtOAc (200 mL). The filtrate was concentrated and then purified by silica gel chromatography (petroleum ether/ethyl acetate=95/5) to give 3-(benzyloxy)-2,4-difluoro-6-((trimethylsilyl)ethynyl)aniline (2.5 g, 79%) as a yellow oil. ¹H NMR (400 MHz, DMSO-d₆): δ 7.46-7.29 (m, 5H), 6.96 (dd, 1H), 5.23-5.11 (m, 4H), 0.23 (s, 9H); LCMS: 332.0 [M+H]⁺.

### Step 5: 6-(Benzyloxy)-5,7-difluoro-1H-indole

A mixture of 3-(benzyloxy)-2,4-difluoro-6-((trimethylsilyl)ethynyl)aniline (2.5 g, 7.54 mmol), t-BuOK (2.96 g, 26.40 mmol), and NMP (50 mL) was stirred at 120° C. for 1 h, allowed to cool to rt, poured into water (100 mL), and then extracted with EtOAc (3×30 mL). The combined organic layers were washed with brine (30 mL), dried over Na₂SO₄, filtered, concentrated, and then purified by silica gel chromatography (petroleum ether/ethyl acetate=95/5) to give 6-(benzyloxy)-5,7-difluoro-1H-indole (1 g, 51%) as a yellow solid. ¹H NMR (400 MHz, DMSO-d₆): δ 11.60 (s, 1H), 7.47-7.42 (m, 2H), 7.42-7.30 (m, 4H), 7.23 (d, 1H), 6.48-6.41 (m, 1H), 5.11 (s, 2H); LCMS: 260.0 [M+H]⁺.

### Intermediate 36

### 5,7-Difluoro-6-methoxyindoline-2,3-dione

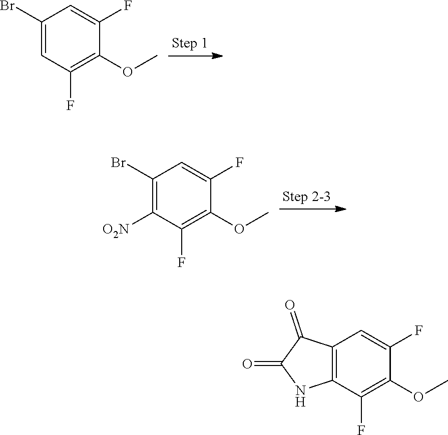

### Step 1: 1-Bromo-3,5-difluoro-4-methoxy-2-nitrobenzene

Potassium nitrate (1.05 g, 10.4 mmol) was added portion-wise (keeping temp between 0-5° C.) to a solution of 5-bromo-1,3-difluoro-2-methoxybenzene (2.25 g, 10.1 mmol) in concentrated H₂SO₄ (20 mL, 37%) at 0° C. The reaction mixture was stirred for 2 h, poured into ice/water (40 mL), and then extracted with CH₂Cl₂ (3×15 mL). The combined organic layers were washed with brine (20 mL), dried over Na₂SO₄, filtered, concentrated, and then purified by chromatography on silica gel (petroleum ether/ethyl acetate=95/5) to give 1-bromo-3,5-difluoro-4-methoxy-2-nitrobenzene (2.3 g, 85%) as a colorless oil. ¹H NMR (400 MHz, DMSO-d₆): δ 7.96 (dd, 1H), 4.04 (s, 3H).

### Step 2: 2,4-Difluoro-3-methoxyaniline

1-Bromo-3,5-difluoro-4-methoxy-2-nitrobenzene (2.85 g, 10.6 mmol) was added to a mixture of Pd/C (10%, 500 mg) and t-BuOH (40 mL) under N₂. The suspension was degassed under vacuum and purged with H₂ several times. The mixture was stirred under H₂ (15 psi) at rt overnight and then filtered. The filter cake was washed with CH₃OH (3×50). The combined organic layers were concentrated and purified by silica gel chromatography (petroleum ether/ethyl acetate=9/1) to give 2,4-difluoro-3-methoxyaniline (1.35 g, 80%) as a yellow oil. ¹H NMR (400 MHz, DMSO-d₆): δ 6.83-6.72 (m, 1H), 6.49-6.36 (m, 1H), 5.01 (s, 2H), 3.86 (s, 3H).

### Step 3: 5,7-Difluoro-6-methoxyindoline-2,3-dione

A mixture of 2,4-difluoro-3-methoxyaniline (1.35 g, 8.48 mmol), concentrated HCl (˜12M, 8.5 mL), water (51 mL), NH₂OH·HCl (1.92 g, 27.6 mmol) and Na₂SO₄ (7.65 g, 53.9 mmol) was stirred at 100° C. for 5 min. 2,2,2-Trichloroethane-1,1-diol (1.68 g, 10.2 mmol) was added, and the mixture was stirred at 100° C. for 1 h. The reaction mixture was allowed to cool to rt and extracted with EtOAc (3×30 mL). The combined organic layers were dried over Na₂SO₄, filtered, concentrated, and then diluted with H₂SO₄ (8.5 mL) and water (0.85 mL). The mixture was stirred at 80° C. for 10 min, allowed to cool to rt, poured onto ice (30 g), and then extracted with EtOAc (3×20 mL). The combined organic layers were washed with brine (20 mL), dried over Na₂SO₄, filtered, concentrated, and then purified by silica gel chromatography (petroleum ether/ethyl acetate=9/1) to give 5,7-difluoro-6-methoxyindoline-2,3-dione (1.2 g, 66%) as a red solid. ¹H NMR (400 MHz, DMSO-d₆): δ 11.57 (s, 1H), 7.50-7.38 (m, 1H), 4.09 (s, 3H); LCMS: 214.1 [M+H]⁺.

### Intermediate 37

### 4,6-Difluoro-5-methoxybenzo[d]thiazol-2(3H)-one

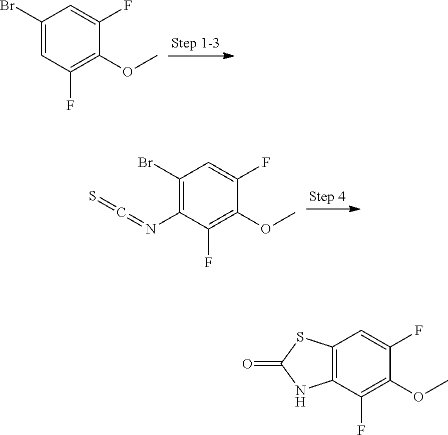

### Step 1: 6-Bromo-2,4-difluoro-3-methoxybenzoic Acid

Lithium diisopropylamide (8.2 mL, 2 M in THF) was added to a solution of 5-bromo-1,3-difluoro-2-methoxybenzene (3.5 g, 16 mmol) in THF (30 mL) at −78° C. under N₂. The reaction mixture was stirred at −78° C. for 1 h. Carbon dioxide (gas) was bubbled into this reaction mixture for 2 h. The mixture was allowed to warm to rt, poured into sat. aq. NH₄Cl (200 mL), and then extracted with EtOAc (3×100 ml). The combined organic layers were washed with brine (300 mL), dried over Na₂SO₄, filtered, concentrated, and then purified by column chromatography (petroleum ether/ethyl acetate=30/70) to obtain 6-bromo-2,4-difluoro-3-methoxybenzoic acid (3.2 g, 76%) as a white solid. ¹H NMR (400 MHz, DMSO-d₆): S 14.18 (s, 1H), 7.66 (dd, 1H), 3.95 (s, 3H); LCMS: 220.8 [(M−45)-H]⁻.

### Step 2: 6-Bromo-2,4-difluoro-3-methoxyaniline

Triethylamine (5.0 mL, 36 mmol) and DPPA (3.63 g, 13.2 mmol) were added to a mixture of 6-bromo-2,4-difluoro-3-methoxybenzoic acid (3.2 g, 12 mmol) in THF (30 mL). The mixture was stirred at rt for 1 h, stirred at 70° C. for 3 h, diluted with H₂O (10 mL), stirred at 70° C. overnight, allowed to cool to rt, poured into H₂O (100 mL), and then extracted with EtOAc (3×100 mL). The combined organic layers were washed with brine (150 mL), dried over Na₂SO₄, filtered, concentrated, and then purified by column chromatography (petroleum ether/ethyl acetate=90/10) to obtain 6-bromo-2,4-difluoro-3-methoxyaniline (1.7 g, 59%) as a white solid. ¹H NMR (400 MHz, DMSO-d₆): δ 7.29 (dd, 1H), 5.19 (s, 2H), 3.88 (s, 3H).

### Step 3: 1-Bromo-3,5-difluoro-2-isothiocyanato-4-methoxybenzene

Thiophosgene (2.17 g, 18.9 mmol) was added to a mixture of 6-bromo-2,4-difluoro-3-methoxyaniline (1.5 g, 6.3 mmol), Na₂CO₃ (4.01 g, 37.8 mmol), and CH₂Cl₂ (30 mL) at 0° C. under N₂. The reaction was stirred at rt overnight, poured into H₂O (100 mL), and then extracted with EtOAc (3×100 mL). The combined organic layers were washed with brine (100 mL), dried over Na₂SO₄, filtered, concentrated, and then purified by column chromatography (petroleum ether/ethyl acetate=10/1) to obtain 1-bromo-3,5-difluoro-2-isothiocyanato-4-methoxybenzene (805 mg, 45%) as a colorless liquid. ¹H NMR (400 MHz, DMSO-d₆): δ 7.75 (dd, 1H), 3.97 (t, 3H).

### Step 4: 4,6-Difluoro-5-methoxybenzo[d]thiazol-2(3H)-one

A mixture of 1-bromo-3,5-difluoro-2-isothiocyanato-4-methoxybenzene (795 mg, 2.86 mmol), CuI (27.2 mg, 0.14 mmol), 1,10-phenanthroline (51.5 mg, 0.29 mmol), K₂CO₃ (790 mg, 5.71 mmol), and EtOH (8 mL) was stirred at 90° C. overnight under N₂ and allowed to cool to rt. Trifluoroacetic acid (16.3 g, 143 mmol) was added. The mixture was stirred at 90° C. overnight, allowed to cool to rt, poured into sat. aq. NaHCO₃ (200 mL), and then extracted with EtOAc (3×200 mL). The combined organic layers were washed with brine (150 mL), dried over Na₂SO₄, filtered, concentrated, and then purified by column chromatography (petroleum ether/ethyl acetate=85/15) to obtain 4,6-difluoro-5-methoxybenzo[d]thiazol-2(3H)-one (495 mg, 80%) as a yellow solid. ¹H NMR (400 MHz, DMSO-d₆): δ 12.46 (s, 1H), 7.53 (dd, 1H), 3.92 (s, 3H); LCMS: 218.0 [M+H]⁺.

### Intermediate 38

### 1,3-Dibromo-4,6-difluoro-5-methoxy-2-(methoxymethoxy)benzene

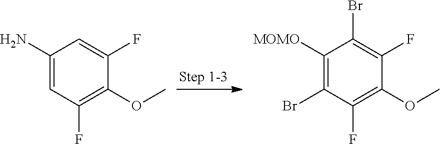

### Step 1: 3,5-Difluoro-4-methoxyphenol

Sodium nitrite (2.30 g, 33.3 mmol) in water (20 mL) was added to a solution of 3,5-difluoro-4-methoxyaniline (5 g, 31.4 mmol) in conc. H₂SO₄ (50% w/w in water, 10 mL) at 0-5° C. The reaction mixture was stirred at 0° C. for 0.5 h and then added dropwise into a stirred mixture of CuSO₄·5H₂O (85 g, 340 mmol), water (100 mL), and xylene (50 mL) at 100° C. The reaction mixture was stirred overnight, allowed to cool to rt, and then extracted with EtOAc (3×30 mL). The combined organic layers were washed with brine (30 mL), dried over Na₂SO₄, filtered, concentrated, and then purified by silica gel chromatography (petroleum ether/EtOAc=9/1) to give 3,5-difluoro-4-methoxyphenol (3 g, 60%) as a yellow oil. ¹H NMR (400 MHz, DMSO-d₆): δ 10.01 (s, 1H), 6.48 (d, 2H), 3.77 (s, 3H); LCMS: 159.0 [M−H]⁻.

### Step 2: 2,6-Dibromo-3,5-difluoro-4-methoxyphenol

Bromine (1.06 mL, 20.6 mmol) was added dropwise to a solution of 3,5-difluoro-4-methoxyphenol (3 g, 19 mmol) in CH₂Cl₂ (30 mL) at 0° C. The mixture was stirred at 0° C. for 30 min, allowed to warm to rt, stirred overnight, poured into sat. aq. Na₂S₂O₃ (50 mL), and then extracted with CH₂Cl₂ (3×50 mL). The combined organic layers were washed with brine (50 mL), dried over Na₂SO₄, filtered, concentrated, and then purified silica gel chromatography (petroleum ether/EtOAc=9/1) to give 2,6-dibromo-3,5-difluoro-4-methoxyphenol (5 g, 84%) as a red oil. ¹H NMR (400 MHz, DMSO-d₆): δ 10.60 (s, 1H), 3.86 (s, 3H); LCMS: 314.7 [M−H]⁻.

### Step 3: 1,3-Dibromo-4,6-difluoro-5-methoxy-2-(methoxymethoxy)benzene

Chloromethyl methyl ether (1.8 mL, 24 mmol) was added to a solution of 2,6-dibromo-3,5-difluoro-4-methoxyphenol (5 g, 16 mmol), DIEA (5.5 mL, 31.5 mmol), and CH₂Cl₂ (50 mL) at 0° C. under N₂. The mixture was allowed to warm to rt, stirred overnight, poured into water (50 mL), and then extracted with CH₂Cl₂ (3×50 mL). The combined organic layers were washed with brine (50 mL), dried over Na₂SO₄, filtered, concentrated, and then purified by silica gel chromatography (petroleum ether/EtOAc=1/100 to 1/20) to give 1,3-dibromo-4,6-difluoro-5-methoxy-2-(methoxymethoxy)benzene (4.5 g, 79%) as a white solid. ¹HNMR (400 MHz, DMSO-d₆): δ 5.15 (s, 2H), 3.94 (s, 3H), 3.61 (s, 3H).

### Intermediate 39

### 6-(Benzyloxy)-2-chloro-5-fluoro-4-(trifluoromethyl)nicotinaldehyde

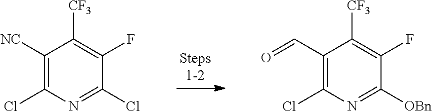 [SMILES: N#Cc1c(Cl)nc(Cl)c(F)c1C(F)(F)F ; O=Cc1c(Cl)nc(OCc2ccccc2)c(F)c1C(F)(F)F]

### Step 1: 6-(Benzyloxy)-2-chloro-5-fluoro-4-(trifluoromethyl)nicotinonitrile

Potassium tert-butoxide (2.76 g, 24.6 mmol) in THF (30 mL) was added dropwise to a solution of phenylmethanol (2.4 mL, 22 mmol) in THF (20 mL) at −78° C. The reaction mixture was stirred at −78° C. for 30 min. 2,6-Dichloro-5-fluoro-4-(trifluoromethyl)nicotinonitrile (5.80 g, 22.4 mmol) in THF (50 mL) was added dropwise at −78° C. The solution was allowed to warm to rt slowly, stirred overnight, poured into H₂O (100 mL), and then extracted with EtOAc (3×100 mL). The combined organic layers were washed with brine (2×100 mL), dried over Na₂SO₄, filtered, concentrated, and then purified by column chromatography (petroleum ether/ethyl acetate=50/1 to 10/1) to obtain 6-(benzyloxy)-2-chloro-5-fluoro-4-(trifluoromethyl)nicotinonitrile (3.70 g, 49%) as a white solid. ¹HNMR (400 MHz, DMSO-d₆): δ 7.52-7.50 (m, 2H), 7.45-7.39 (m, 3H), 5.55 (s, 2H); LCMS: 331.1 [M+H]⁺.

### Step 2: 6-(Benzyloxy)-2-chloro-5-fluoro-4-(trifluoromethyl)nicotinaldehyde

Diisobutylaluminum hydride (13.4 mL, 13.4 mmol, 1 M in toluene) was added to a solution of 6-(benzyloxy)-2-chloro-5-fluoro-4-(trifluoromethyl)nicotinonitrile (3.70 g, 11.2 mmol) in toluene (50 mL) at −78° C. The mixture was stirred at −78° C. for 1 h, allowed to warm to rt, poured into sat. aq. NH₄Cl (50 mL), and then extracted with EtOAc (3×30 mL). The combined organic layers were washed with brine (2×30 mL), dried over Na₂SO₄, filtered, concentrated, and then purified by column chromatography (petroleum ether/ethyl acetate=20/1 to 5/1) to obtain 6-(benzyloxy)-2-chloro-5-fluoro-4-(trifluoromethyl)nicotinaldehyde (850 mg, 22%) as a white solid. ¹H NMR (400 MHz, DMSO-d₆): δ 10.27 (s, 1H), 7.52-7.50 (m, 2H), 7.44-7.38 (m, 3H), 5.53 (s, 2H); LCMS: 334.1 [M+H]⁺.

### Intermediate 40

### 1-(4-Bromophenyl)-4-chloro-5-fluoro-1H-benzo[d][1,2,3]triazole

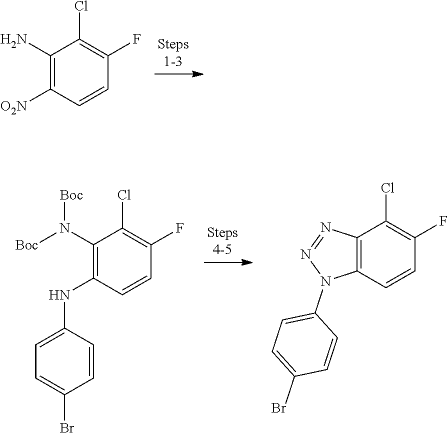 [SMILES: Nc1c([N+](=O)[O-])ccc(F)c1Cl ; CC(C)(C)OC(=O)N(C(=O)OC(C)(C)C)c1c(Nc2ccc(Br)cc2)ccc(F)c1Cl ; Fc1ccc2c(nnn2-c2ccc(Br)cc2)c1Cl]

### Step 1: tert-Butyl N-tert-butoxy car bonyl-N-(2-chloro-3-fluoro-6-nitro-phenyl)carbamate

4-(Dimethylamino)pyridine (692 mg, 5.67 mmol) was added slowly in portions to a mixture of 2-chloro-3-fluoro-6-nitroaniline (10.8 g, 56.7 mmol), Boc₂O (24.7 g, 113 mmol) and THF (150 mL) at rt. The reaction mixture was stirred at rt overnight, poured into water (300 mL), and then extracted with EtOAc (3×200 mL). The combined organic layers were washed with brine (200 mL), dried over Na₂SO₄, filtered, concentrated, and then purified by silica gel chromatography (petroleum ether/EtOAc=9/1) to give tert-butyl N-tert-butoxycarbonyl-N-(2-chloro-3-fluoro-6-nitro-phenyl)carbamate (18 g, 81%) as a light yellow solid. ¹HNMR (400 MHz, DMSO-d₆): δ 8.35-8.23 (m, 1H), 7.90-7.77 (m, 1H), 1.33 (s, 18H).

### Step 2: tert-Butyl N-(6-amino-2-chloro-3-fluoro-phenyl)-N-tert-butoxycarbonyl-carbamate

tert-Butyl N-tert-butoxycarbonyl-N-(2-chloro-3-fluoro-6-nitro-phenyl)carbamate (2 g, 5.12 mmol) was added to a mixture of Pd/C (0.5 g, 10%) in THF (20 mL) under N₂. The suspension was degassed under vacuum and purged with H₂ several times. The mixture was stirred under H₂ (15 psi) at rt for 5 h. The mixture was combined with two other reaction mixtures of the same scale and filtered through Celite. The filter cake was washed with EtOAc (500 mL). The organic layer was concentrated to give crude tert-butyl N-(6-amino-2-chloro-3-fluoro-phenyl)-N-tert-butoxycarbonyl-carbamate (6 g) as a yellow oil. LCMS: 205.1 [(M-Boc-t-butyl)+H]⁺.

### Step 3: Di-tert-butyl (6-((4-bromophenyl)amino)-2-chloro-3-fluorophenyl)iminodicarbonate

Copper(II) acetate (4.53 g, 24.9 mmol) was added to a mixture of tert-butyl N-(6-amino-2-chloro-3-fluoro-phenyl)-N-tert-butoxycarbonyl-carbamate (6 g, 17 mmol), (4-bromophenyl)boronic acid (6.68 g, 33.3 mmol), pyridine (2.70 mL, 33.3 mmol), and CH₂Cl₂ (100 mL) at rt. The reaction mixture was stirred overnight under O₂ atmosphere (˜15 psi) and then filtered through Celite. The filter cake was washed with EtOAc (300 mL). The filtrate was concentrated and then purified by silica gel chromatography (petroleum ether/EtOAc=3/1) to give di-tert-butyl (6-((4-bromophenyl)amino)-2-chloro-3-fluorophenyl)iminodicarbonate (4.5 g) as a red oil. LCMS: 358.9 [(M-Boc-t-butyl)+H]⁺.

### Step 4: N^{V}-(4-Bromophenyl)-3-chloro-4-fluorobenzene-1,2-diamine

A mixture of di-tert-butyl (6-((4-bromophenyl)amino)-2-chloro-3-fluorophenyl)iminodicarbonate (8.1 g, 16 mmol), TFA (20 mL), and CH₂Cl₂ (100 mL) was stirred at rt overnight, concentrated, diluted with sat. aq. NaHCO₃ (100 mL), and then extracted with CH₂Cl₂ (3×50 mL). The combined organic layers were washed with brine (50 mL), dried over Na₂SO₄, filtered, concentrated, and then purified by silica gel chromatography (petroleum ether/EtOAc=4/1) to give N¹-(4-bromophenyl)-3-chloro-4-fluorobenzene-1,2-diamine (2.5 g, 28% over 3 steps) as a yellow solid. ¹H NMR (400 MHz, DMSO-d₆): δ 7.45 (s, 1H), 7.26 (d, 2H), 7.01-6.92 (m, 1H), 6.63-6.49 (m, 3H), 5.30 (s, 2H); LCMS: 315.0 [M+H]⁺.

### Step 5: 1-(4-Bromophenyl)-4-chloro-5-fluoro-1H-benzo[d][1,2,3]triazole

Sulfuric acid (7.5 mL) and H₂O (67.5 mL) were added dropwise to a solution of N¹-(4-bromophenyl)-3-chloro-4-fluorobenzene-1,2-diamine (2.5 g, 7.9 mmol) in THF (25 mL) at 0° C. Sodium nitrite (765 mg, 11.1 mmol) in H₂O (7.5 mL) was added dropwise to the mixture at 0° C. The reaction mixture was stirred at 0° C. for 15 min, diluted with water (100 mL), and then extracted with EtOAc (3×50 mL). The combined organic layers were washed with brine (50 mL), dried over Na₂SO₄, filtered, and then concentrated. The crude was triturated with petroleum ether/EtOAc=3/1 (50 mL) at rt overnight and then filtered. The filter cake was washed with ice-cold petroleum ether/EtOAc=5/1 (15 mL) and then dried under high vacuum to give 1-(4-bromophenyl)-4-chloro-5-fluoro-1H-benzo[d][1,2,3]triazole (1.7 g, 66%) as a red solid. ¹H NMR (400 MHz, DMSO-d₆): δ 8.00-7.94 (m, 1H), 7.93-7.89 (m, 2H), 7.88-7.83 (m, 2H), 7.82-7.74 (m, 1H); LCMS: 326.0 [M+H]⁺.

The Intermediates below were synthesized from the appropriate starting materials following the procedures described for Intermediate 40.

| Int | Structure | Name | [M + H]⁺ |
|---|---|---|---|
| 40.1 | 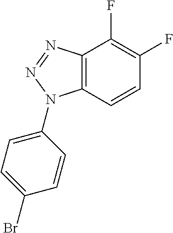 [SMILES: Fc1ccc2c(nnn2-c2ccc(Br)cc2)c1F] | 1-(4-Bromophenyl)-4,5-difluoro- 1H-benzo[d][1,2,3]triazole | 310.0 |
| 40.2^{1,2} | 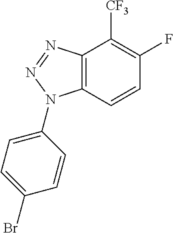 [SMILES: Fc1ccc2c(nnn2-c2ccc(Br)cc2)c1C(F)(F)F] | 1-(4-Bromophenyl)-5-fluoro-4- (trifluoromethyl)-1H- benzo[d][1,2,3]triazole | 359.9 |

Alternate conditions used:
Step 2: reaction time was 5 h-ON;
¹Step 2: NH₄Cl, Fe, CH₃OH, H₂O, 70° C., 1 h;
²Step 4: 4 M HCl in EtOAc, rt, 1 h.

### Compound 1

### 1-(4′-Hydroxy-[1,1′-biphenyl]-4-yl)-1H-indazol-6-ol

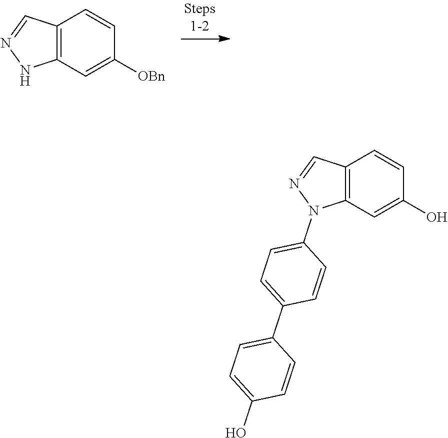 [SMILES: c1ccc(COc2ccc3cn[nH]c3c2)cc1 ; Oc1ccc(-c2ccc(-n3ncc4ccc(O)cc43)cc2)cc1]

### Step 1: 6-(Benzyloxy)-1-(4′-(benzyloxy)-[1,1′-biphenyl]-4-yl)-1H-indazole

4-(Benzyloxy)-4′-bromo-1,1′-biphenyl (138 mg, 0.62 mmol) was added to a mixture of 6-(benzyloxy)-1H-indazole (200 mg, 0.59 mmol), tris(dibenzylideneacetone)dipalladium(0) (32 mg, 0.035 mmol), tBuXPhos (54 mg, 0.13 mmol), sodium tert-butoxide (87 mg, 0.91 mmol), and toluene (4 mL). The mixture was degassed with 2 vacuum/N₂ cycles, stirred at 100° C. for 7 h, allowed to cool to rt, and then diluted with CH₂Cl₂ (10 mL) and water (5 mL). Celite was added, and the mixture was filtered through Celite. The filter cake was washed with CH₂Cl₂ (5 mL). The organic layer was dried (Na₂SO₄), filtered, and then concentrated. The crude solid was suspended in EtOAc (5 mL) and hexanes (5 mL), and the mixture was stirred at rt for 15.5 h. The solids were filtered and washed with 1:1 EtOAc/hexanes (2×3 mL) to give 6-(benzyloxy)-1-(4′-(benzyloxy)-[1,1′-biphenyl]-4-yl)-1H-indazole (162 mg, 54%) as a greyish white solid. ¹H NMR (400 MHz, DMSO-d₆): δ 8.27 (s, 1H), 7.86-7.75 (m, 5H), 7.71 (d, J=8.7 Hz, 2H), 7.53-7.47 (m, 4H), 7.47-7.39 (m, 4H), 7.39-7.33 (m, 3H), 7.15 (d, J=8.8 Hz, 2H), 6.99 (dd, J=2.0, 8.8 Hz, 1H), 5.24 (s, 2H), 5.19 (s, 2H); LCMS: 483.0 [M+H]⁺.

### Step 2: 1-(4′-Hydroxy-[1,1′-biphenyl]-4-yl)-1H-indazol-6-ol

Palladium on carbon (5 wt. %, 34 mg, 0.016 mmol) was added to a mixture of 6-(benzyloxy)-1-(4′-(benzyloxy)-[1,1′-biphenyl]-4-yl)-1H-indazole (157 mg, 0.33 mmol), CH₃OH (2 mL), and EtOAc (2 mL) at rt under N₂. The mixture was degassed with 3 vacuum/H₂ cycles and stirred at rt for 22 h. Celite was added, and the mixture was filtered through Celite, washed with CH₃OH (10 mL), and then concentrated. The crude solid was suspended in CH₃OH (2 mL) and minimal CH₂Cl₂, and then stirred at rt for 1 h. The solids were filtered and washed with CH₃OH (3 mL) to give 1-(4′-hydroxy-[1,1′-biphenyl]-4-yl)-1H-indazol-6-ol (64 mg, 59%) as a pink powder. ¹H NMR (400 MHz, DMSO-d₆): δ 9.89 (s, 1H), 9.61 (s, 1H), 8.19 (d, J=0.7 Hz, 1H), 7.80-7.71 (m, 4H), 7.68 (d, J=8.7 Hz, 1H), 7.60-7.55 (m, 2H), 7.13 (br t, 1H), 6.91-6.87 (m, 2H), 6.80 (dd, J=1.8, 8.7 Hz, 1H); LCMS: 302.9 [M+H]⁺.

### Compound 2

### 5-Fluoro-1-(3′-hydroxy-[1,1′-biphenyl]-4-yl)-1H-indazol-6-ol

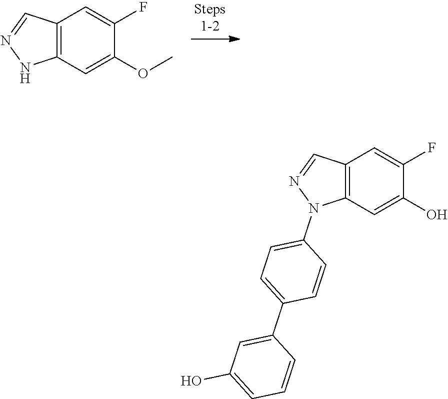 [SMILES: COc1cc2[nH]ncc2cc1F ; Oc1cccc(-c2ccc(-n3ncc4cc(F)c(O)cc43)cc2)c1]

### Step 1: 5-Fluoro-6-methoxy-1-(3′-methoxy-[1,1′-biphenyl]-4-yl)-1H-indazole

1-(4-Bromophenyl)-3-methoxybenzene (174 mg, 0.66 mmol) was added to a mixture of 5-fluoro-6-methoxy-1H-indazole (100 mg, 0.60 mmol), tris(dibenzylideneacetone)dipalladium(0) (29 mg, 0.032 mmol), tBuXPhos (53 mg, 0.13 mmol), sodium tert-butoxide (87 mg, 0.91 mmol), and toluene (3 mL). The mixture was degassed with 1 vacuum/N₂ cycle, stirred at 100° C. for 20 h, allowed to cool to rt, and then diluted with CH₂Cl₂ (10 mL) and water (5 mL). Celite was added, and the mixture and filtered through Celite. The organic layer was dried (Na₂SO₄), filtered, concentrated, and then purified by silica gel chromatography (0-20% EtOAc in hexanes) to give 5-fluoro-6-methoxy-1-(3′-methoxy-[1,1′-biphenyl]-4-yl)-1H-indazole (134 mg, 64%) as a beige powder. ¹H NMR (400 MHz, DMSO-d₆): δ 8.28 (s, 1H), 7.93-7.87 (m, 4H), 7.72 (d, J=10.9 Hz, 1H), 7.46-7.40 (m, 2H), 7.33 (d, J=7.8 Hz, 1H), 7.29 (t, J=2.0 Hz, 1H), 6.98 (dd, J=2.2, 7.8 Hz, 1H), 3.98 (s, 3H), 3.86 (s, 3H); LCMS: 348.9 [M+H]⁺.

### Step 2: 5-Fluoro-1-(3′-hydroxy-[1,1′-biphenyl]-4-yl)-1H-indazol-6-ol

Boron tribromide (1 M in CH₂Cl₂, 3.04 mL, 3.04 mmol) was added slowly to a mixture of 5-fluoro-6-methoxy-1-(3′-methoxy-[1,1′-biphenyl]-4-yl)-1H-indazole (106 mg, 0.30 mmol) in CH₂Cl₂ (1 mL) at −78° C. under N₂. The mixture was allowed to warm to rt, stirred for 21 h, cooled in an ice/water bath, and then quenched slowly with CH₃OH (1 mL). The mixture was concentrated and then purified by reverse-phase HPLC (40-65% CH₃CN in water with 0.1% TFA). The product fractions were combined, and CH₃CN was removed. The aqueous layer was basified with sat'd NaHCO₃ and then extracted with EtOAc (10 mL). The organic layer was washed with brine (10 mL), dried (Na₂SO₄), filtered, and then concentrated to give 5-fluoro-1-(3′-hydroxy-[1,1′-biphenyl]-4-yl)-1H-indazol-6-ol (69 mg, 67%) as a pale pink solid. ¹HNMR (400 MHz, DMSO-d₆): δ 10.45 (s, 1H), 9.60 (s, 1H), 8.22 (s, 1H), 7.83-7.76 (m, 4H), 7.64 (d, J=10.6 Hz, 1H), 7.35-7.28 (m, 2H), 7.16 (d, J=8.1 Hz, 1H), 7.10 (t, J=2.0 Hz, 1H), 6.81 (dd, J=1.8, 8.0 Hz, 1H); LCMS: 320.9 [M+H]⁺.

The Compounds below were synthesized from the appropriate indazole and the appropriate aryl bromide following the procedures described for Compound 2.

| Cmpd | Structure | Name | [M + H]⁺ |
|---|---|---|---|
| 2.1² | 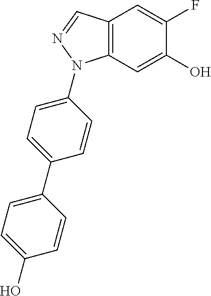 [SMILES: Oc1ccc(-c2ccc(-n3ncc4cc(F)c(O)cc43)cc2)cc1] | 5-Fluoro-1-(4′-hydroxy-[1,1′- biphenyl]-4-yl)-1H-indazol-6-ol | 320.9 |
| 2.2 | 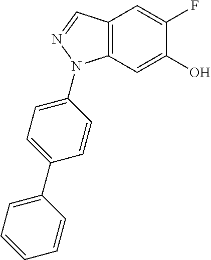 [SMILES: Oc1cc2c(cnn2-c2ccc(-c3ccccc3)cc2)cc1F] | 1-([1,1′-Biphenyl]-4-yl)-5-fluoro- 1H-indazol-6-ol | 304.9 |
| 2.3 | 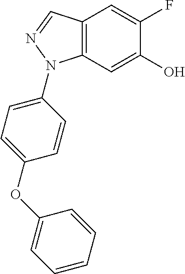 [SMILES: Oc1cc2c(cnn2-c2ccc(Oc3ccccc3)cc2)cc1F] | 5-Fluoro-1-(4-phenoxyphenyl)- 1H-indazol-6-ol | 320.9 |
| 2.4² | 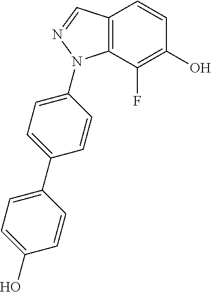 [SMILES: Oc1ccc(-c2ccc(-n3ncc4ccc(O)c(F)c43)cc2)cc1] | 7-Fluoro-1-(4′-hydroxy-[1,1′- biphenyl]-4-yl)-1H-indazol-6-ol | 320.9 |
| 2.5 | 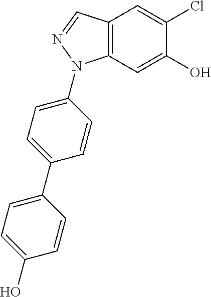 [SMILES: Oc1ccc(-c2ccc(-n3ncc4cc(Cl)c(O)cc43)cc2)cc1] | 5-Chloro-1-(4′-hydroxy-[1,1′- biphenyl]-4-yl)-1H-indazol-6-ol | 366.8 |
| 2.6 | 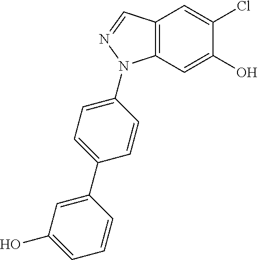 [SMILES: Oc1cccc(-c2ccc(-n3ncc4cc(Cl)c(O)cc43)cc2)c1] | 5-Chloro-1-(3′-hydroxy-[1,1′- biphenyl]-4-yl)-1H-indazol-6-ol | 366.9 |
| 2.7¹ | 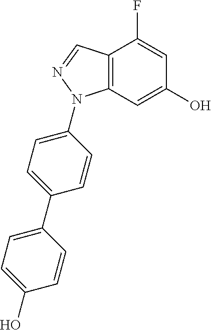 [SMILES: Oc1ccc(-c2ccc(-n3ncc4c(F)cc(O)cc43)cc2)cc1] | 4-Fluoro-1-(4′-hydroxy-[1,1′- biphenyl]-4-yl)-1H-indazol-6-ol | 320.9 |
| 2.8 | 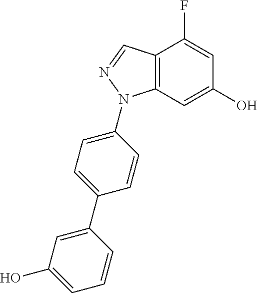 [SMILES: Oc1cccc(-c2ccc(-n3ncc4c(F)cc(O)cc43)cc2)c1] | 4-Fluoro-1-(3′-hydroxy-[1,1′- biphenyl]-4-yl)-1H-indazol-6-ol | 320.9 |
| 2.9³ | 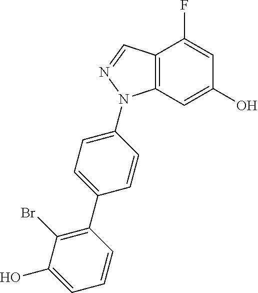 [SMILES: Oc1cc(F)c2cnn(-c3ccc(-c4cccc(O)c4Br)cc3)c2c1] | 1-(2′-Bromo-3′-hydroxy-[1,1′- biphenyl]-4-yl)-4-fluoro-1H- indazol-6-ol | 398.9 |

Alternate conditions:
Step 1: 4-21 h; microwave, 120° C., 20 min.
Step 2: 2-22 h; also 35° C., 17 h; in some instances, additional BBr₃ was needed (up to 10 eq);
¹Pyridine HCl, 180° C., 4 h.
²1-(4-Bromophenyl)-4-(phenylmethoxy)benzene used in Step 1.
³Isolated during the purification of Compound 2.8 (bromination occurred in DMSO solution used for HPLC injection).

### Compound 3

### 5-Fluoro-1-(4-(phenylamino)phenyl)-1H-indazol-6-ol

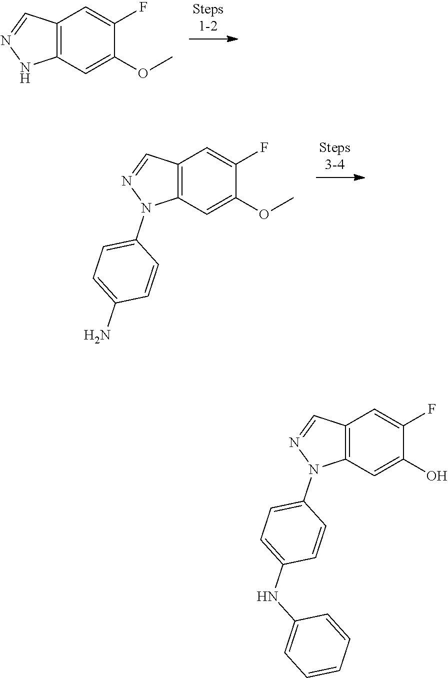 [SMILES: COc1cc2[nH]ncc2cc1F ; COc1cc2c(cnn2-c2ccc(N)cc2)cc1F ; Oc1cc2c(cnn2-c2ccc(Nc3ccccc3)cc2)cc1F]

### Step 1: 5-Fluoro-6-methoxy-1-(4-nitrophenyl)-1H-indazole

1-Bromo-4-nitrobenzene (134 mg, 0.66 mmol) was added to a mixture of 5-fluoro-6-methoxy-1H-indazole (100 mg, 0.60 mmol), tris(dibenzylideneacetone)dipalladium(0) (28 mg, 0.030 mmol), tBuXPhos (51 mg, 0.12 mmol), sodium tert-butoxide (87 mg, 0.91 mmol), and toluene (2 mL). The mixture was degassed with 2 vacuum/N₂ cycles, stirred at 100° C. for 16 h, allowed to cool to rt, and then diluted with CH₂Cl₂ (10 mL) and water (5 mL). Celite was added, and the mixture was filtered through Celite. The organic layer was dried (Na₂SO₄), filtered, and then concentrated. The solids were suspended in CH₂Cl₂ (1 mL), EtOAc (1 mL), and then hexanes (5 mL). The solids were filtered and then washed with 1:1 EtOAc/hexanes (2×5 mL) to give 5-fluoro-6-methoxy-1-(4-nitrophenyl)-1H-indazole (64 mg 37%) as a brown solid. ¹H NMR (400 MHz, DMSO-d₆): δ 8.44 (d, J=9.0 Hz, 2H), 8.40 (s, 1H), 8.15 (d, J=9.0 Hz, 2H), 7.77 (d, J=10.6 Hz, 1H), 7.57 (d, J=7.0 Hz, 1H), 4.01 (s, 3H); LCMS: 287.8 [M+H]⁺.

### Step 2: 4-(5-Fluoro-6-methoxy-1H-indazol-1-yl)aniline

Palladium on carbon (10 wt. %, 12 mg) was added to a mixture of 5-fluoro-6-methoxy-1-(4-nitrophenyl)-1H-indazole (60 mg, 0.21 mmol), CH₃OH (2.5 mL), and EtOAc (2.5 mL) at rt under N₂. The mixture was degassed with 3 vacuum/H₂ cycles, stirred at rt for 65 h, and then filtered through Celite. The filter cake was washed with CH₃OH (2 mL), and the filtrate was concentrated to give 4-(5-fluoro-6-methoxy-1H-indazol-1-yl)aniline (47 mg, 87%) as a light brown solid. LCMS: 257.9 [M+H]⁺.

### Step 3: 4-(5-Fluoro-6-methoxy-1H-indazol-1-yl)-N-phenylaniline

Bromobenzene (57 mg, 0.37 mmol) was added to a mixture of 4-(5-fluoro-6-methoxy-1H-indazol-1-yl)aniline (47 mg, 0.18 mmol), palladium(II) acetate (12 mg, 0.053 mmol), XantPhos (42 mg, 0.07 mmol), cesium carbonate (180 mg, 0.55 mmol), and 1,4-dioxane (2 mL). The mixture was degassed with 1 vacuum/N₂ cycle, heated at 85° C. for 17 h, cooled to rt, and then diluted with EtOAc (10 mL). The organic layer was washed with water (10 mL), washed with brine (10 mL), dried (Na₂SO₄), filtered, concentrated, and then purified by silica gel chromatography (0-40% EtOAc in hexanes) to give 4-(5-fluoro-6-methoxy-1H-indazol-1-yl)-N-phenylaniline (41 mg, 67%) as an off-white foam. ¹H NMR (400 MHz, DMSO-d₆): δ 8.43 (s, 1H), 8.17 (s, 1H), 7.67 (d, J=11.0 Hz, 1H), 7.59 (d, J=8.8 Hz, 2H), 7.32-7.22 (m, 5H), 7.16 (d, J=7.7 Hz, 2H), 6.89 (t, J=7.2 Hz, 1H), 3.93 (s, 3H); LCMS: 334.0 [M+H]⁺.

### Step 4: 5-Fluoro-1-(4-(phenylamino)phenyl)-1H-indazol-6-ol

Boron tribromide (1 M in CH₂Cl₂, 0.35 mL, 0.35 mmol) was added slowly to a mixture of 4-(5-fluoro-6-methoxy-1H-indazol-1-yl)-N-phenylaniline (39 mg, 0.12 mmol) in CH₂Cl₂ (2 mL) at −78° C. under N₂. The mixture was allowed to warm to rt, stirred for 24 h, and then cooled in a dry ice/acetone bath. Additional boron tribromide (1 M in CH₂Cl₂, 0.35 mL, 0.35 mmol) was added slowly. The mixture was allowed to warm to rt, stirred for 71 h, cooled in a dry ice/acetone bath, quenched slowly with CH₃OH (1 mL), concentrated, and then purified by reverse-phase HPLC (40-60% CH₃CN in 0.1% TFA in water). The product fractions were combined, and CH₃CN was removed. The aqueous layer was basified with sat'd NaHCO₃ (10 mL) and extracted with CH₂Cl₂ (10 mL). The organic layer was dried (Na₂SO₄), filtered, and then concentrated to give 5-fluoro-1-(4-(phenylamino)phenyl)-1H-indazol-6-ol (22 mg, 59%) as a beige solid. ¹HNMR (400 MHz, DMSO-d₆): δ 10.34 (br s, 1H), 8.41 (s, 1H), 8.11 (s, 1H), 7.59 (d, J=10.8 Hz, 1H), 7.54-7.47 (m, 2H), 7.31-7.21 (m, 4H), 7.18-7.12 (m, 3H), 6.88 (t, J=7.3 Hz, 1H); LCMS: 319.9 [M+H]⁺.

### Compound 4

### 5-Fluoro-1-(3′-hydroxy-[1,1′-biphenyl]-4-yl)-4-(trifluoromethyl)-1H-indazol-6-ol

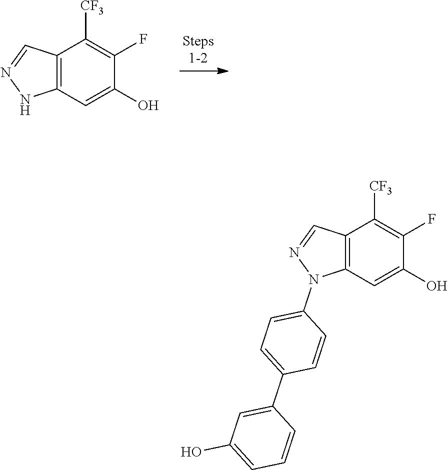 [SMILES: Oc1cc2[nH]ncc2c(C(F)(F)F)c1F ; Oc1cccc(-c2ccc(-n3ncc4c(C(F)(F)F)c(F)c(O)cc43)cc2)c1]

### Step 1: 5-Fluoro-1-(3′-methoxy-[1,1′-biphenyl]-4-yl)-4-(trifluoromethyl)-1H-indazol-6-ol

A mixture of Intermediate 11 (400 mg, 1.82 mmol), 1-bromo-4-(3-methoxyphenyl)benzene (478 mg, 1.82 mmol), BrettPhos Pd G4 (167 mg, 0.181 mmol), t-BuONa (524 mg, 5.45 mmol), and DME (20 mL) was refluxed overnight under N₂, cooled to rt, poured into water (50 mL) and extracted with EtOAc (2×60 mL). The combined organic layers were washed with water (2×40 mL), washed with brine (40 mL), dried (Na₂SO₄), filtered, concentrated, and then purified by silica gel chromatography (petroleum ether/EtOAc=20/1) to give 5-fluoro-1-(3′-methoxy-[1,1′-biphenyl]-4-yl)-4-(trifluoromethyl)-1H-indazol-6-ol (250 mg, 33%) as a yellow solid. ¹H NMR (400 MHz, DMSO-d₆): δ 11.13 (s, 1H), 8.34 (d, 1H), 7.91 (d, 2H), 7.78 (d, 2H), 7.55 (d, 1H), 7.46-7.38 (m, 1H), 7.35-7.25 (m, 2H), 6.99 (dd, 1H), 3.95-3.72 (s, 3H); LCMS: 403.1 [M+H]⁺.

### Step 2: 5-Fluoro-1-(3′-hydroxy-[1,1′-biphenyl]-4-yl)-4-(trifluoromethyl)-1H-indazol-6-ol

Boron tribromide (2.60 g, 10.38 mmol) was added to a solution of 5-fluoro-1-(3′-methoxy-[1,1′-biphenyl]-4-yl)-4-(trifluoromethyl)-1H-indazol-6-ol (250 mg, 0.621 mmol) in CH₂Cl₂ (20 mL) at −78° C. The mixture was allowed to warm to 0° C. for 1 h, stirred at rt overnight, added dropwise to CH₃OH (˜30 mL) at 0° C., and then concentrated. The residue was diluted with EtOAc (30 mL). The organics were washed with NaHCO₃ (30 mL), washed with water (2×20 mL), washed with brine (20 mL), dried (Na₂SO₄), filtered, concentrated, and then purified by reverse-phase HPLC [water(0.04% HCl)—CH₃CN] to give 5-fluoro-1-(3′-hydroxy-[1,1′-biphenyl]-4-yl)-4-(trifluoromethyl)-1H-indazol-6-ol (80.6 mg, 330%) as a yellow solid. ¹H NMR (400 MHz, DMSO-d₆): δ 11.13 (s, 1H), 9.61 (s, 1H), 8.35 (d, 1H), 7.88-7.82 (m, 2H), 7.82-7.75 (m, 2H), 7.57 (d, 1H), 7.35-7.27 (m, 1H), 7.17 (d, 1H), 7.11 (d, 1H), 6.82 (dd, 1H); LCMS: 388.9 [M+H]⁺.

The Compounds below were synthesized from the appropriate Intermediates and the appropriate starting materials following the procedures described for Compound 4.

| Cmpd | Structure | Name | [M + H]⁺ |
|---|---|---|---|
| 4.1¹ | 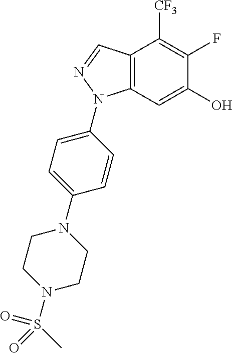 [SMILES: CS(=O)(=O)N1CCN(c2ccc(-n3ncc4c(C(F)(F)F)c(F)c(O)cc43)cc2)CC1] | 5-Fluoro-1-(4-(4- (methylsulfonyl)piperazin-1- yl)phenyl)-4-(trifluoromethyl)-1H- indazol-6-ol | 459.0 |
| 4.2 | 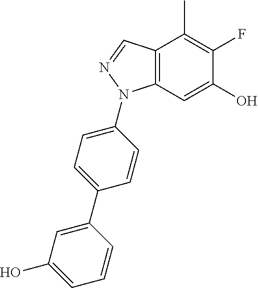 [SMILES: Cc1c(F)c(O)cc2c1cnn2-c1ccc(-c2cccc(O)c2)cc1] | 5-Fluoro-1-(3′-hydroxy-[1,1′- biphenyl]-4-yl)-4-methyl-1H- indazol-6-ol | 335.0 |
| 4.3 | 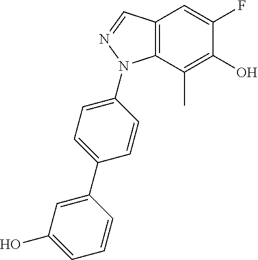 [SMILES: Cc1c(O)c(F)cc2cnn(-c3ccc(-c4cccc(O)c4)cc3)c12] | 5-Fluoro-1-(3′-hydroxy-[1,1′- biphenyl]-4-yl)-7-methyl-1H- indazol-6-ol | 335.0 |

¹From Intermediate 11 and 1-(4-bromophenyl)-4-(methylsulfonyl)piperazine (110° C., 10 h); Step 1 only.

### Compound 5

### 4-Chloro-5-fluoro-1-(3′-hydroxy-[1,1′-biphenyl]-4-yl)-1H-indazol-6-ol

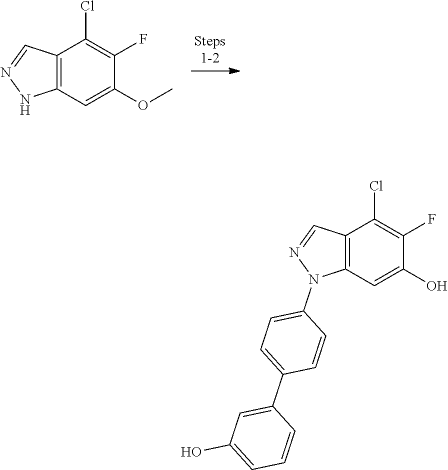 [SMILES: COc1cc2[nH]ncc2c(Cl)c1F ; Oc1cccc(-c2ccc(-n3ncc4c(Cl)c(F)c(O)cc43)cc2)c1]

### Step 1: 4-Chloro-5-fluoro-6-methoxy-1-(3′-methoxy-[1,1′-biphenyl]-4-yl)-1H-indazole

Copper(I) iodide (19.0 mg, 0.100 mmol) was added to a solution of Intermediate 12 (500 mg, crude), 4′-bromo-3-methoxy-1,1′-biphenyl (262 mg, 0.997 mmol), trans-N¹,N²-dimethylcyclohexane-1,2-diamine (70.9 mg, 0.499 mmol), K₃PO₄ (381 mg, 1.79 mmol), and dioxane (5 mL) under N₂. The mixture was stirred at 100° C. overnight, cooled to rt, poured into water (20 mL), and then extracted with EtOAc (3×10 mL). The combined organic layers were washed with brine (10 mL), dried over Na₂SO₄, filtered, concentrated, and then purified by silica gel chromatography (petroleum ether/EtOAc=92/8) to give 4-chloro-5-fluoro-6-methoxy-1-(3′-methoxy-[1,1′-biphenyl]-4-yl)-1H-indazole (150 mg) as a white solid. ¹H NMR (400 MHz, DMSO-d₆): δ 8.41 (s, 1H), 8.04-7.73 (m, 4H), 7.52-7.37 (m, 2H), 7.35-7.23 (m, 2H), 6.98 (dd, 1H), 4.12-3.73 (m, 6H); LCMS: 383.0 [M+H]⁺.

### Step 2: 4-Chloro-5-fluoro-1-(3′-hydroxy-[1,1′-biphenyl]-4-yl)-1H-indazol-6-ol

Boron tribromide (2.23 g, 8.88 mmol) was added dropwise to a solution of 4-chloro-5-fluoro-6-methoxy-1-(3′-methoxy-[1,1′-biphenyl]-4-yl)-1H-indazole (170 mg, 0.444 mmol) in CH₂Cl₂ (4 mL) at −78° C. under N₂. The reaction mixture was warmed to rt, stirred overnight, and then added dropwise to dry CH₃OH (20 mL) at 0° C. The mixture was diluted with saturated NaHCO₃ (40 mL) and extracted with CH₂Cl₂ (3×20 mL). The combined organic layers were washed with brine (20 mL), dried over Na₂SO₄, filtered, concentrated, and then purified by prep-HPLC [water (0.04% HCl)/CH₃CN] to give 4-chloro-5-fluoro-1-(3′-hydroxy-[1,1′-biphenyl]-4-yl)-1H-indazol-6-ol (72.1 mg, 45%) as a light yellow solid. ¹H NMR (400 MHz, DMSO-d₆): δ 10.91 (s, 1H), 9.59 (s, 1H), 8.33 (s, 1H), 7.88-7.80 (m, 2H), 7.79-7.71 (m, 2H), 7.36-7.23 (m, 2H), 7.15 (d, 1H), 7.10 (s, 1H), 6.81 (dd, 1H); LCMS: 355.0 [M+H]⁺.

The Compounds below were synthesized from the appropriate Intermediate and the appropriate aryl bromide following the procedures described for Compound 5.

| Cmpd | Structure | Name | [M + H]⁺ |
|---|---|---|---|
| 5.1 | 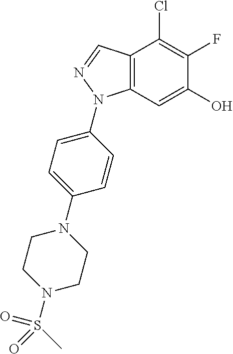 [SMILES: CS(=O)(=O)N1CCN(c2ccc(-n3ncc4c(Cl)c(F)c(O)cc43)cc2)CC1] | 4-Chloro-5-fluoro-1-(4-(4- (methylsulfonyl)piperazin-1- yl)phenyl)-1H-indazol-6-ol | 425.0 |
| 5.2 | 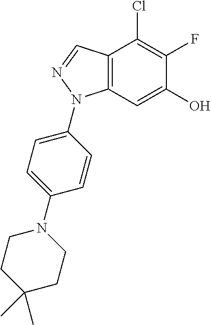 [SMILES: CC1(C)CCN(c2ccc(-n3ncc4c(Cl)c(F)c(O)cc43)cc2)CC1] | 4-Chloro-1-(4-(4,4- dimethylpiperidin-1-yl)phenyl)-5- fluoro-1H-indazol-6-ol | 374.1 |
| 5.3 | 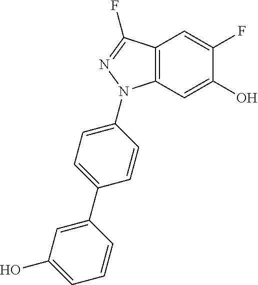 [SMILES: Oc1cccc(-c2ccc(-n3nc(F)c4cc(F)c(O)cc43)cc2)c1] | 3,5-Difluoro-1-(3′-hydroxy-[1,1′- biphenyl]-4-yl)-1H-indazol-6-ol | 339.1 |
| 5.4 | 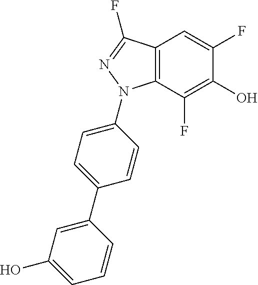 [SMILES: Oc1cccc(-c2ccc(-n3nc(F)c4cc(F)c(O)c(F)c43)cc2)c1] | 3,5,7-Trifluoro-1-(3′-hydroxy- [1,1′-biphenyl]-4-yl)-1H-indazol- 6-ol | 357.0 |

Alternate conditions:
Step 2: −78° C., 1 h then rt, 2 h; pyridine hydrochloride, 180° C., 1.5 h.

### Compound 6

### 3,5,7-Trifluoro-1-(4-(4-(methylsulfonyl)piperazin-1-yl)phenyl)-1H-indazol-6-ol

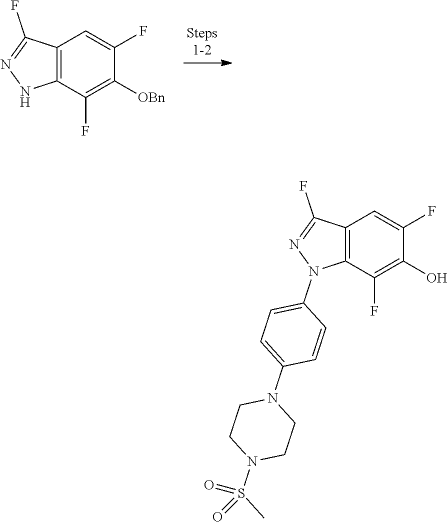 [SMILES: Fc1cc2c(F)n[nH]c2c(F)c1OCc1ccccc1 ; CS(=O)(=O)N1CCN(c2ccc(-n3nc(F)c4cc(F)c(O)c(F)c43)cc2)CC1]

### Step 1: 6-(Benzyloxy)-3,5,7-trifluoro-1-(4-(4-(methylsulfonyl)piperazin-1-yl)phenyl)-1H-indazole

Copper(I) iodide (6.16 mg, 32.3 μmol) was added to a solution of Intermediate 6 (90 mg, 323 μmol), 1-(4-bromophenyl)-4-(methylsulfonyl)piperazine (114 mg, 356 μmol), K₃PO₄ (124 mg, 582 μmol), and trans-N,N-dimethylcyclohexane-1,2-diamine (23.1 mg, 162 μmol) in dioxane (1 mL) under N₂. The mixture was stirred at 100° C. for 12 h, cooled to rt, filtered, concentrated, and then purified by prep-TLC (SiO₂, petroleum ether/ethyl acetate=1:1) to give 6-(benzyloxy)-3,5,7-trifluoro-1-(4-(4-(methylsulfonyl)piperazin-1-yl)phenyl)-1H-indazole (100 mg, 60%) as a yellow solid. LCMS: 517.2 [M+H]⁺.

### Step 2: 3,5,7-Trifluoro-1-(4-(4-(methylsulfonyl)piperazin-1-yl)phenyl)-1H-indazol-6-ol

Palladium on carbon (10%, 100 mg, 0.094 mmol) was added to a solution of 6-(benzyloxy)-3,5,7-trifluoro-1-(4-(4-(methylsulfonyl)piperazin-1-yl)phenyl)-1H-indazole (100 mg, 0.194 mmol) in CH₃OH (20 mL). The mixture was stirred under H₂ at rt for 3 h, filtered, concentrated, and then purified by prep-HPLC [water (0.04% HCl)—CH₃CN] to give 3,5,7-trifluoro-1-(4-(4-(methylsulfonyl)piperazin-1-yl)phenyl)-1H-indazol-6-ol (43.2 mg, 52%) as a white solid. ¹H NMR (400 MHz, DMSO-d₆): δ 10.76-11.02 (m, 1H), 7.55 (d, 1H), 7.39-7.45 (m, 2H), 7.10 (d, 2H), 3.31-3.38 (m, 4H), 3.22-3.30 (m, 4H), 2.94 (s, 3H); LCMS: 427.0 [M+H]⁺.

The Compound below was synthesized from Intermediates following the procedures described for Compound 6.

| Cmpd | Structure | Name | [M + H]⁺ |
|---|---|---|---|
| 6.1 | 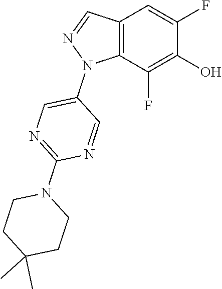 [SMILES: CC1(C)CCN(c2ncc(-n3ncc4cc(F)c(O)c(F)c43)cn2)CC1] | 1-(2-(4,4-Dimethylpiperidin-1- yl)pyrimidin-5-yl)-5,7- difluoro-1H-indazol-6-ol | 360.2 |
| 6.2 | 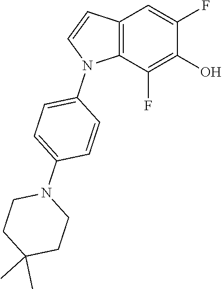 [SMILES: CC1(C)CCN(c2ccc(-n3ccc4cc(F)c(O)c(F)c43)cc2)CC1] | 1-(4-(4,4-Dimethylpiperidin- 1-yl)phenyl)-5,7-difluoro- 1H-indol-6-ol | 357.2 |

### Compound 7

### 5,7-Difluoro-1-(2-(4-(methylsulfonyl)piperazin-1-yl)pyrimidin-5-yl)-1H-indazol-6-ol

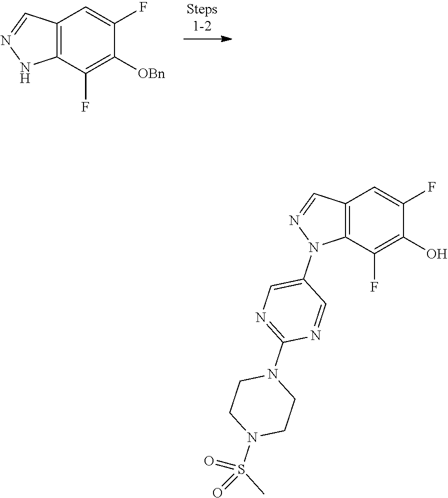 [SMILES: Fc1cc2cn[nH]c2c(F)c1OCc1ccccc1 ; CS(=O)(=O)N1CCN(c2ncc(-n3ncc4cc(F)c(O)c(F)c43)cn2)CC1]

### Step 1: 6-(Benzyloxy)-5,7-difluoro-1-(2-(4-(methylsulfonyl)piperazin-1-yl)pyrimidin-5-yl)-1H-indazole

A mixture of Intermediate 5 (260 mg, 0.999 mmol), Intermediate 30 (425 mg, 1.49 mmol), pyridine (158 mg, 2.00 mmol), Cu(OAc)₂ hydrate (299 mg, 1.50 mmol), and CH₂Cl₂ (20 mL) was stirred under O₂ (15 psi) at rt overnight, poured into ammonium hydroxide (10 mL), and then extracted with EtOAc (3×10 mL). The combined organic layers were washed with brine (10 mL), dried over Na₂SO₄, filtered, concentrated, and then purified by silica gel chromatography (petroleum ether/EtOAc=80/20) to give 6-(benzyloxy)-5,7-difluoro-1-(2-(4-(methylsulfonyl)piperazin-1-yl)pyrimidin-5-yl)-1H-indazole (200 mg) as a yellow solid. ¹H NMR (400 MHz, DMSO-d₆): δ 8.69 (d, 2H), 8.40 (s, 1H), 7.65 (d, 1H), 7.50-7.31 (m, 5H), 5.22 (s, 2H), 3.99-3.92 (m, 4H), 3.27-3.20 (m, 4H), 2.92 (s, 3H); LCMS: 501.0 [M+H]⁺.

### Step 2: 5,7-Difluoro-1-(2-(4-(methylsulfonyl)piperazin-1-yl)pyrimidin-5-yl)-1H-indazol-6-ol

6-(Benzyloxy)-5,7-difluoro-1-(2-(4-(methylsulfonyl)piperazin-1-yl)pyrimidin-5-yl)-1H-indazole (200 mg) was added to a mixture of 10% Pd/C (0.1 g) and CH₃OH (20 mL) under N₂. The suspension was degassed with several vacuum/H₂ cycles, stirred under H₂ (15 psi) at rt for 2 h, and then filtered through Celite. The filter cake was washed with a 1:1 mixture of CH₂Cl₂/CH₃OH (200 mL). The organic phase was collected, concentrated, and then purified by prep-HPLC [water (0.04% HCl)/CH₃CN] to give 5,7-difluoro-1-(2-(4-(methylsulfonyl)piperazin-1-yl)pyrimidin-5-yl)-1H-indazol-6-ol (131.2 mg, 32% over 2 steps) as a white solid. ¹H NMR (400 MHz, DMSO-d₆): δ 10.62 (s, 1H), 8.67 (d, 2H), 8.30 (d, 1H), 7.53 (dd, 1H), 3.99-3.91 (m, 4H), 3.27-3.22 (m, 4H), 2.91 (s, 3H); LCMS: 411.0 [M+H]⁺.

The Compounds below were synthesized from the appropriate Intermediates following the procedures described for Compound 7.

| Cmpd | Structure | Name | [M + H]⁺ |
|---|---|---|---|
| 7.1¹ | 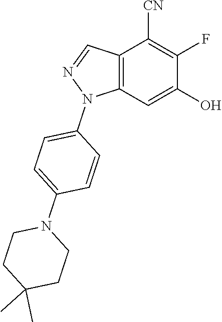 [SMILES: CC1(C)CCN(c2ccc(-n3ncc4c(C#N)c(F)c(O)cc43)cc2)CC1] | 1-(4-(4,4-Dimethylpiperidin-1- yl)phenyl)-5-fluoro-6-hydroxy- 1H-indazole-4-carbonitrile | 365.6 |
| 7.2¹ | 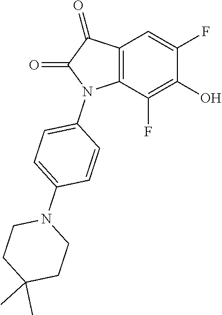 [SMILES: CC1(C)CCN(c2ccc(N3C(=O)C(=O)c4cc(F)c(O)c(F)c43)cc2)CC1] | 1-(4-(4,4-Dimethylpiperidin-1- yl)phenyl)-5,7-difluoro-6- hydroxyindoline-2,3-dione | 387.5 |
| 7.3 | 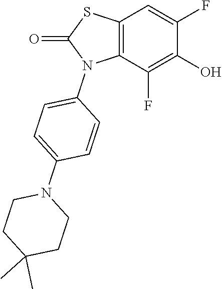 [SMILES: CC1(C)CCN(c2ccc(-n3c(=O)sc4cc(F)c(O)c(F)c43)cc2)CC1] | 3-(4-(4,4-Dimethylpiperidin-1- yl)phenyl)-4,6-difluoro-5- hydroxybenzo[d]thiazol-2(3H)- one | 391.3 |
| 7.4² | 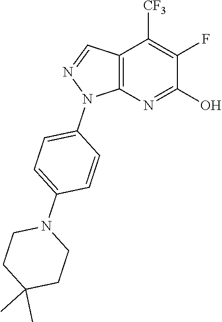 [SMILES: CC1(C)CCN(c2ccc(-n3ncc4c(C(F)(F)F)c(F)c(O)nc43)cc2)CC1] | 1-(4-(4,4-Dimethylpiperidin-1- yl)phenyl)-5-fluoro-4- (trifluoromethyl)-1H-pyrazolo[3,4- b]pyridin-6-ol | 409.5 |

Alternate conditions:
¹Step 2: BBr₃, CH₂Cl₂, reflux, 32 h or −78° C. to rt, 1-2 h;
²Step 2: TFA, 70° C., 1 h.

### Compound 8

### 4,5-Difluoro-1-(3′-hydroxy-[1,1′-biphenyl]-4-yl)-1H-indazol-6-ol

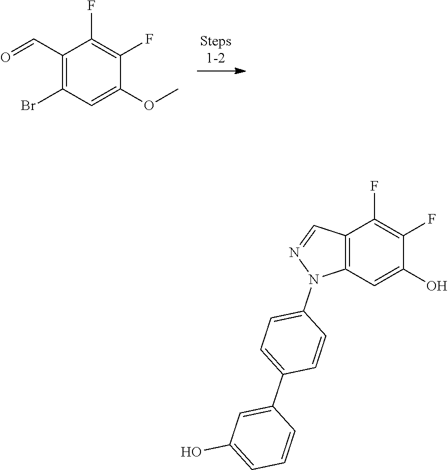 [SMILES: COc1cc(Br)c(C=O)c(F)c1F ; Oc1cccc(-c2ccc(-n3ncc4c(F)c(F)c(O)cc43)cc2)c1]

### Step 1: 4,5-Difluoro-6-methoxy-1-(3′-methoxy-[1,1′-biphenyl]-4-yl)-1H-indazole

A mixture of 6-bromo-2,3-difluoro-4-methoxybenzaldehyde (300 mg, 1.20 mmol), Intermediate 28 (270 mg, 1.26 mmol), and EtOH (5 mL) was refluxed at 80° C. for 1 h, allowed to cool to rt, and then concentrated to dryness. The residue was added to a mixture of Pd₂(dba)₃ (109 mg, 0.119 mmol), BINAP (74.4 mg, 0.119 mmol), K₃PO₄ (761 mg, 3.59 mmol), and toluene (10 mL). The mixture was stirred at 110° C. overnight under N₂, allowed to cool to rt, poured into water (15 mL), and then extracted with ethyl acetate (2×20 mL). The combined organics were washed with water (20 mL), washed with brine (20 mL), dried (Na₂SO₄), filtered, concentrated, and then purified by silica gel chromatography (petroleum ether/ethyl acetate=20/1 to 10/1) to give 4,5-difluoro-6-methoxy-1-(3′-methoxy-[1,1′-biphenyl]-4-yl)-1H-indazole (180 mg, 41%) as a yellow solid. ¹H NMR (400 MHz, CDCl₃): δ 8.14 (s, 1H), 7.75-7.63 (m, 4H), 7.37-7.29 (m, 1H), 7.19-7.16 (m, 1H), 7.12-7.09 (m, 1H), 6.92-6.84 (m, 2H), 3.90 (s, 3H), 3.82 (s, 3H); LCMS: 367.2 [M+H]⁺.

### Step 2: 4,5-Difluoro-1-(3′-hydroxy-[1,1′-biphenyl]-4-yl)-1H-indazol-6-ol

A mixture of 4,5-difluoro-6-methoxy-1-(3′-methoxy-[1,1′-biphenyl]-4-yl)-1H-indazole (180 mg, 0.491 mmol) and pyridine hydrochloride (9.0 g, 78 mmol) was stirred at 180° C. for 1 h, allowed to cool to rt, quenched with water (10 mL), and then extracted with ethyl acetate (2×10 mL). The combined organic layers were washed with H₂O (2×10 mL), dried over Na₂SO₄, concentrated, and then purified by reverse-phase HPLC [water (0.04% HCl)—CH₃CN] to give 4,5-difluoro-1-(3′-hydroxy-[1,1′-biphenyl]-4-yl)-1H-indazol-6-ol (52.6 mg, 32%) as a yellow solid. ¹H NMR (400 MHz, DMSO-d₆): δ 10.95 (s, 1H), 9.60 (s, 1H), 8.43 (s, 1H), 7.92-7.67 (m, 4H), 7.40-7.22 (m, 1H), 7.23-7.01 (m, 3H), 6.82 (dd, 1H); LCMS: 339.1 [M+H]⁺.

### Compound 9

### 5-Fluoro-1-(4′-hydroxy-[1,1′-biphenyl]-4-yl)-1H-benzo[d]imidazol-6-ol

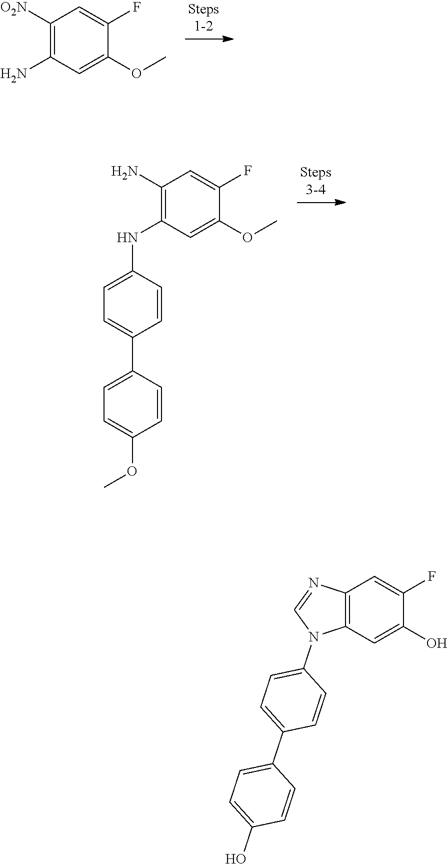 [SMILES: COc1cc(N)c([N+](=O)[O-])cc1F ; COc1ccc(-c2ccc(Nc3cc(OC)c(F)cc3N)cc2)cc1 ; Oc1ccc(-c2ccc(-n3cnc4cc(F)c(O)cc43)cc2)cc1]

### Step 1: N-(4-Fluoro-5-methoxy-2-nitrophenyl)-4′-methoxy-[1,1′-biphenyl]-4-amine

A mixture of 4-fluoro-5-methoxy-2-nitroaniline (155 mg, 0.84 mmol), 1-bromo-4-(4-methoxyphenyl)benzene (200 mg, 0.76 mmol), tris(dibenzylideneacetone)dipalladium(0) (35 mg, 0.04 mmol), XPhos (36 mg, 0.08 mmol), sodium tert-butoxide (146 mg, 1.52 mmol), and toluene (2 mL) was degassed with 2 vacuum/N₂ cycles, stirred at 100° C. for 16 h, and then allowed to cool to rt. The mixture was diluted with EtOAc (15 mL) and water (10 mL). Celite was added, and the mixture was filtered through Celite. The filter cake was washed with EtOAc (10 mL). The organic layer was washed with brine (10 mL), dried (Na₂SO₄), filtered, concentrated, and then purified by silica gel chromatography (0-15% EtOAc in hexanes) to give N-(4-fluoro-5-methoxy-2-nitrophenyl)-4′-methoxy-[1,1′-biphenyl]-4-amine (73 mg, 26%) as an orange solid. ¹HNMR (400 MHz, DMSO-d₆): δ 9.74 (s, 1H), 8.02 (d, J=11.9 Hz, 1H), 7.67 (m, 4H), 7.48 (d, J=8.6 Hz, 2H), 7.04 (d, J=8.8 Hz, 2H), 6.85 (d, J=7.8 Hz, 1H), 3.83-3.81 (m, 3H), 3.81-3.80 (m, 3H); LCMS: 369.2 [M+H]⁺.

### Step 2: 4-Fluoro-5-methoxy-N¹-(4′-methoxy-[1,1′-biphenyl]-4-yl)benzene-1,2-diamine

Palladium on carbon (5 wt. %) was added to a mixture of N-(4-fluoro-5-methoxy-2-nitrophenyl)-4′-methoxy-[1,1′-biphenyl]-4-amine (115 mg, 0.31 mmol) and EtOAc (2 mL) at rt under N₂. The mixture was degassed with 3 vacuum/H₂ cycles, stirred at rt for 64 h, and then filtered. The filter cake was washed with EtOAc (5 mL), and the filtrate was concentrated to give 4-fluoro-5-methoxy-N¹-(4′-methoxy-[1,1′-biphenyl]-4-yl)benzene-1,2-diamine (109 mg) as a purple solid. ¹H NMR (400 MHz, DMSO-d₆): δ 7.49 (d, J=8.7 Hz, 2H), 7.39 (d, J=8.6 Hz, 2H), 7.23 (s, 1H), 6.96 (d, J=8.8 Hz, 2H), 6.81 (d, J=9.0 Hz, 1H), 6.72 (d, J=8.7 Hz, 2H), 6.62 (d, J=13.3 Hz, 1H), 4.64 (br s, 2H), 3.81-3.76 (m, 3H), 3.68 (s, 3H); LCMS: 338.9 [M+H]⁺.

### Step 3: 5-Fluoro-6-methoxy-1-(4′-methoxy-[1,1′-biphenyl]-4-yl)-1H-benzo[d]imidazole

A mixture of 4-fluoro-5-methoxy-N′-(4′-methoxy-[1,1′-biphenyl]-4-yl)benzene-1,2-diamine (53 mg, 0.16 mmol), formic acid (50 μL, 1.33 mmol), 1,4-dioxane (0.5 mL), and water (0.5 mL) was stirred at 100° C. for 20 h, cooled to rt, diluted with 1.0 M NaOH (1 mL), and then extracted with EtOAc (10 mL). The organic layer washed with brine (10 mL), dried (Na₂SO₄), filtered, concentrated, and then purified by silica gel chromatography (0-30% EtOAc in CH₂Cl₂) to give 5-fluoro-6-methoxy-1-(4′-methoxy-[1,1′-biphenyl]-4-yl)-1H-benzo[d]imidazole (21 mg, 38%) as a purple solid. ¹H NMR (400 MHz, DMSO-d₆): δ 8.50 (s, 1H), 7.88 (d, J=8.6 Hz, 2H), 7.78-7.70 (m, 4H), 7.66 (d, J=11.6 Hz, 1H), 7.29 (d, J=7.7 Hz, 1H), 7.11-7.06 (m, 2H), 3.90 (s, 3H), 3.83 (s, 3H); LCMS: 349.2 [M+H]⁺.

### Step 4: 5-Fluoro-1-(4′-hydroxy-[1,1′-biphenyl]-4-yl)-1H-benzo[d]imidazol-6-ol

5-Fluoro-6-methoxy-1-(4′-methoxy-[1,1′-biphenyl]-4-yl)-1H-benzo[d]imidazole (19 mg, 0.05 mmol) and pyridine hydrochloride (206 mg, 1.78 mmol) was stirred at 170° C. for 3.5 h, cooled to rt, and then diluted with 1.0 M HCl (1 mL). A mixture of EtOAc/CH₂Cl₂ was added, and the solids were filtered and then purified by reverse-phase HPLC (20-60% CH₃CN in 0.1% TFA in water). The product fractions were combined, concentrated, and dissolved in EtOAc. The organic layer washed with sat'd NaHCO₃ (10 mL), washed with brine (10 mL), dried (Na₂SO₄), filtered, and then concentrated to give 5-fluoro-1-(4′-hydroxy-[1,1′-biphenyl]-4-yl)-1H-benzo[d]imidazol-6-ol (3.7 mg, 22%) as an off-white solid. ¹H NMR (400 MHz, DMSO-d₆): δ 9.88 (s, 1H), 9.66 (s, 1H), 8.42 (s, 1H), 7.82 (d, J=8.6 Hz, 2H), 7.66 (d, J=8.4 Hz, 2H), 7.62-7.54 (m, 3H), 7.13 (d, J=7.8 Hz, 1H), 6.90 (d, J=8.7 Hz, 2H); LCMS: 320.9 [M+H]⁺.

### Compound 10

### 5-Fluoro-1-(4′-hydroxy-[1,1′-biphenyl]-4-yl)-1H-benzo[d][1,2,3]triazol-6-ol

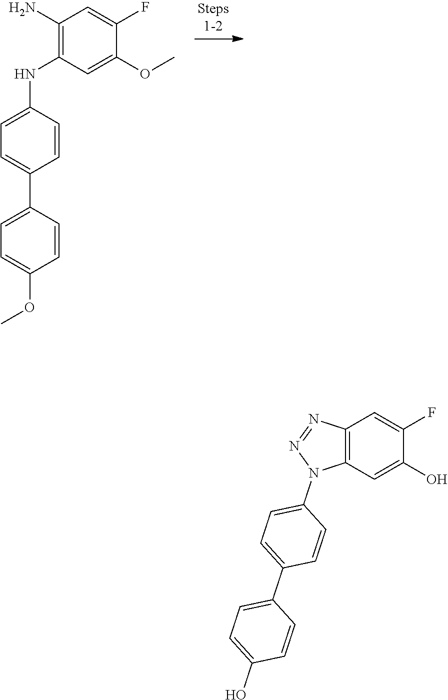 [SMILES: COc1ccc(-c2ccc(Nc3cc(OC)c(F)cc3N)cc2)cc1 ; Oc1ccc(-c2ccc(-n3nnc4cc(F)c(O)cc43)cc2)cc1]

### Step 1: 5-Fluoro-6-methoxy-1-(4′-methoxy-[1,1′-biphenyl]-4-yl)-1H-benzo[d][1,2,3]triazole

Concentrated sulfuric acid (0.2 mL) and water (1.8 mL) was added to Compound 9, Step 2 (55 mg, 0.16 mmol) in THF (1 mL) at 0° C. Sodium nitrite (16 mg, 0.23 mmol) in water (0.25 mL) was added dropwise. The mixture was stirred at 0° C. for 20 min, poured into water (10 mL), and then extracted with EtOAc (2×10 mL). The combined organic layers were washed with brine (10 mL), dried (Na₂SO₄), filtered, concentrated, and then purified by silica gel chromatography (0-20% EtOAc in CH₂Cl₂) to give 5-fluoro-6-methoxy-1-(4′-methoxy-[1,1′-biphenyl]-4-yl)-1H-benzo[d][1,2,3]triazole (32 mg, 56%) as a peach solid. ¹HNMR (400 MHz, DMSO-d₆): δ 8.10 (d, J=10.5 Hz, 1H), 7.94 (s, 4H), 7.75 (d, J=8.7 Hz, 2H), 7.47 (d, J=7.2 Hz, 1H), 7.10 (d, J=8.7 Hz, 2H), 4.01 (s, 3H), 3.83 (s, 3H); LCMS: 349.3 [M+H]⁺.

### Step 2: 5-Fluoro-1-(4′-hydroxy-[1,1′-biphenyl]-4-yl)-1H-benzo[d][1,2,3]triazol-6-ol

5-Fluoro-6-methoxy-1-(4′-methoxy-[1,1′-biphenyl]-4-yl)-1H-benzo[d][1,2,3]triazole (29 mg, 0.08 mmol) and pyridine hydrochloride (290 mg, 2.51 mmol) were stirred at 170° C. for 6 h, cooled to rt, and diluted with 1.0 M HCl (2 mL). EtOAc was added, and the aqueous layer was pipetted off. The organic layer was concentrated and purified by reverse-phase HPLC (30-70% CH₃CN in 0.1% TFA in water). The product fractions were combined, concentrated, and dissolved in EtOAc (10 mL). The organic layer was washed with sat'd NaHCO₃ (10 mL), washed with brine (10 mL), dried (Na₂SO₄), filtered, and then concentrated to give 5-fluoro-1-(4′-hydroxy-[1,1′-biphenyl]-4-yl)-1H-benzo[d][1,2,3]triazol-6-ol (6.9 mg, 26%) as a pink solid. ¹H NMR (400 MHz, DMSO-d₆): δ 10.88 (s, 1H), 9.69 (s, 1H), 8.02 (d, J=10.5 Hz, 1H), 7.91-7.86 (m, 2H), 7.86-7.81 (m, 2H), 7.63 (d, J=8.6 Hz, 2H), 7.28 (d, J=7.5 Hz, 1H), 6.91 (d, J=8.7 Hz, 2H); LCMS: 321.9 [M+H]⁺.

### Compound 11

### 1-(4-(4,4-Dimethylpiperidin-1-yl)phenyl)-5,7-difluoro-6-hydroxy-1H-benzo[d]imidazol-2-2(3H)-one

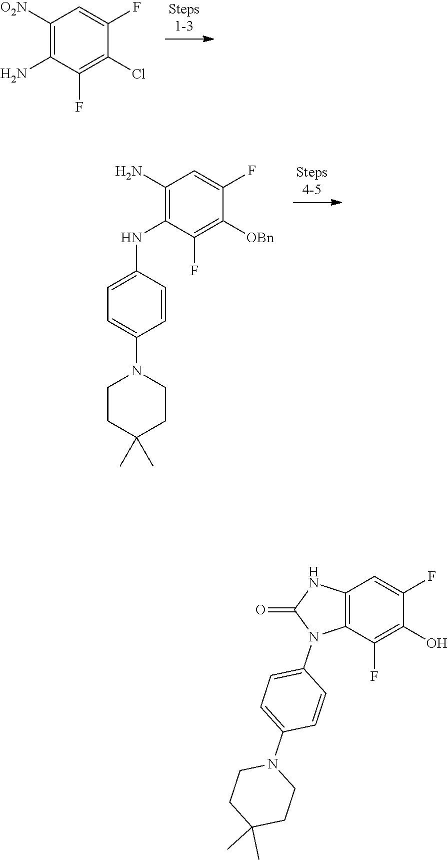 [SMILES: Nc1c([N+](=O)[O-])cc(F)c(Cl)c1F ; CC1(C)CCN(c2ccc(Nc3c(N)cc(F)c(OCc4ccccc4)c3F)cc2)CC1 ; CC1(C)CCN(c2ccc(-n3c(=O)[nH]c4cc(F)c(O)c(F)c43)cc2)CC1]

### Step 1: 3-(Benzyloxy)-2,4-difluoro-6-nitroaniline

Potassium hydroxide (800 mg, 14.26 mmol) was added to benzyl alcohol (5 mL, 48.3 mmol) and DMSO (2 mL). The mixture was stirred at 70° C. for 1.5 h and cooled to rt. 3-Chloro-2,4-difluoro-6-nitrophenylamine (2.0 g, 9.59 mmol) was added. The reaction was stirred at 65° C. for 1 h, allowed to cool to rt, and diluted with CH₂Cl₂ (60 mL). The organics were washed with water (20 mL), dried (Na₂SO₄), filtered, concentrated, and then purified by silica gel chromatography (0-15% EtOAc in hexanes) to give 3-(benzyloxy)-2,4-difluoro-6-nitroaniline (779 mg, 29%) as an orange solid. ¹H NMR (400 MHz, DMSO-d₆) δ 7.79 (dd, J=2.1, 12.2 Hz, 1H), 7.49-7.33 (m, 5H), 7.31 (s, 2H), 5.36 (s, 2H).

### Step 2: 3-(Benzyloxy)-N-(4-(4,4-dimethylpiperidin-1-yl)phenyl)-2,4-difluoro-6-nitroaniline

A mixture of 3-(benzyloxy)-2,4-difluoro-6-nitroaniline (190 mg, 0.68 mmol), Intermediate 29 (200 mg, 0.75 mmol), tris(dibenzylideneacetone)dipalladium (125 mg, 0.14 mmol), XPhos (65 mg, 0.14 mmol), sodium tert-butoxide (153 mg, 1.59 mmol), and toluene (2 mL) was degassed with 2 vacuum/N₂ cycles. The mixture stirred at 100° C. for 1 h, allowed to cool to rt, diluted with EtOAc (20 mL) and water (10 mL), and then filtered through Celite.

The filter cake was washed with EtOAc (5 mL) and water (5 mL). The organic layer was separated, washed with brine (10 mL), dried (Na₂SO₄), filtered, concentrated, and then purified by silica gel chromatography (0-20% EtOAc in CH₂Cl₂) to give 3-(benzyloxy)-N-(4-(4,4-dimethylpiperidin-1-yl)phenyl)-2,4-difluoro-6-nitroaniline (85 mg, 27%) as a red solid. ¹H NMR (400 MHz, DMSO-d₆) δ 8.54 (s, 1H), 7.95 (br d, J=11.5 Hz, 1H), 7.44-7.38 (m, 5H), 6.87-6.75 (m, 4H), 5.32 (s, 2H), 3.13-3.00 (m, 4H), 1.48-1.41 (m, 4H), 0.96 (s, 6H); LCMS: 468.1 [M+H]⁺.

### Step 3: 5-(Benzyloxy)-N¹-(4-(4,4-dimethylpiperidin-1-yl)phenyl)-4,6-difluorobenzene-1,2-diamine

A mixture of 3-(benzyloxy)-N-(4-(4,4-dimethylpiperidin-1-yl)phenyl)-2,4-difluoro-6-nitroaniline (82 mg, 0.18 mmol), tin(II) chloride dihydrate (120 mg, 0.53 mmol), and EtOH (2 mL) was stirred at 70° C. for 1 h, allowed to cool to rt, concentrated, and then diluted with EtOAc (10 mL) and 1.0 M NaOH (10 mL). The mixture was stirred at rt for 10 min. Celite was added. The mixture was filtered through Celite, and the filter cake was washed with EtOAc (2×10 mL). The organic layer was separated, washed with brine (10 mL), dried (Na₂SO₄), filtered, and then concentrated to give 5-(benzyloxy)-N¹-(4-(4,4-dimethylpiperidin-1-yl)phenyl)-4,6-difluorobenzene-1,2-diamine (64 mg, 83%) as a purple solid. ¹HNMR (400 MHz, DMSO-d₆) δ 7.40-7.34 (m, 5H), 6.75 (d, J=8.8 Hz, 2H), 6.67 (s, 1H), 6.40-6.34 (m, 3H), 5.07 (s, 2H), 4.95 (s, 2H), 2.96-2.88 (m, 4H), 1.49-1.38 (m, 4H), 0.94 (s, 6H); LCMS: 438.3 [M+H]⁺.

### Step 4: 6-(Benzyloxy)-1-(4-(4,4-dimethylpiperidin-1-yl)phenyl)-5,7-difluoro-1H-benzo[d]imidazol-2(3H)-one

Pyridine (22 μL, 0.27 mmol) was added to a mixture of 5-(benzyloxy)-N′-(4-(4,4-dimethylpiperidin-1-yl)phenyl)-4,6-difluorobenzene-1,2-diamine (60 mg, 0.14 mmol), 1,1′-carbonyldiimidazole (44 mg, 0.27 mmol), and THF (1 mL). The reaction was stirred at rt for 3 h, stirred at 50° C. for 2 h, allowed to cool to rt, concentrated, and then purified by silica gel chromatography (0-25% EtOAc in CH₂Cl₂). The product fractions were combined and concentrated. The solids were suspended in CH₂Cl₂ (2 mL), sonicated for 30 sec, and then filtered. The filter cake was washed with CH₂Cl₂ (2×1 mL) to give 6-(benzyloxy)-1-(4-(4,4-dimethylpiperidin-1-yl)phenyl)-5,7-difluoro-1H-benzo[d]imidazol-2(3H)-one (50 mg, 79%) as a fluffy white solid. ¹H NMR (400 MHz, DMSO-d₆) δ 11.32 (s, 1H), 7.42-7.32 (m, 5H), 7.22 (br d, J=7.3 Hz, 2H), 7.00 (d, J=9.0 Hz, 2H), 6.88-6.82 (m, 1H), 5.01 (s, 2H), 3.26-3.20 (m, 4H), 1.50-1.43 (m, 4H), 0.98 (s, 6H); LCMS: 464.4 [M+H]⁺.

### Step 5: 1-(4-(4,4-Dimethylpiperidin-1-yl)phenyl)-5,7-difluoro-6-hydroxy-1H-benzo[d]imidazol-2(3H)-one

Palladium on carbon (10 wt. %) was added to a mixture of 6-(benzyloxy)-1-(4-(4,4-dimethylpiperidin-1-yl)phenyl)-5,7-difluoro-1H-benzo[d]imidazol-2(3H)-one (23 mg, 0.05 mmol) and THF (3 mL) at rt under N₂. The mixture was degassed with 3 vacuum/H₂ cycles, stirred for 1 h, and then filtered. The filter cake was washed with THF (1 mL), and the filtrate was concentrated to give 1-(4-(4,4-dimethylpiperidin-1-yl)phenyl)-5,7-difluoro-6-hydroxy-1H-benzo[d]imidazol-2(3H)-one (18 mg, 91%) as an off-white solid. ¹H NMR (400 MHz, DMSO-d₆) δ 11.06 (s, 1H), 9.49 (s, 1H), 7.22 (br d, J=7.5 Hz, 2H), 7.00 (d, J=8.9 Hz, 2H), 6.77 (dd, J=1.3, 9.8 Hz, 1H), 3.26-3.18 (m, 4H), 1.50-1.43 (m, 4H), 0.98 (s, 6H); LCMS: 374.1 [M+H]⁺.

### Compound 12

### 3-(4-(4,4-Dimethylpiperidin-1-yl)phenyl)-1-ethyl-4,6-difluoro-5-hydroxy-LH-benzo[d]imidazol-2(3H)-one

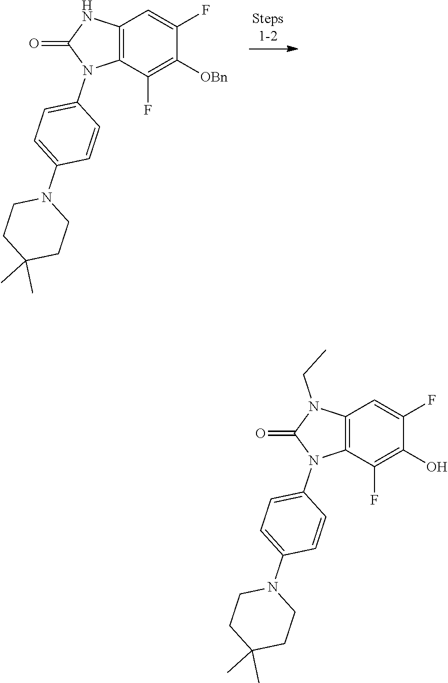 [SMILES: CC1(C)CCN(c2ccc(-n3c(=O)[nH]c4cc(F)c(OCc5ccccc5)c(F)c43)cc2)CC1 ; CCn1c(=O)n(-c2ccc(N3CCC(C)(C)CC3)cc2)c2c(F)c(O)c(F)cc21]

### Step 1: 5-(Benzyloxy)-3-(4-(4,4-dimethylpiperidin-1-yl)phenyl)-1-ethyl-4,6-difluoro-1H-benzo[d]imidazol-2(3H)-one

A mixture of Compound 11, Step 4 (25 mg, 0.054 mmol), cesium carbonate (35 mg 0.11 mmol), DMF (1 mL), and iodoethane (6.5 μL, 0.08 mmol) was stirred at rt for 2 h, diluted with water (10 mL), and then extracted with EtOAc (10 mL). The organic layer was washed with brine (10 mL), dried (Na₂SO₄), filtered, concentrated, and then purified by silica gel chromatography (0-50% EtOAc in hexanes) to give 5-(benzyloxy)-3-(4-(4,4-dimethylpiperidin-1-yl)phenyl)-1-ethyl-4,6-difluoro-1H-benzo[d]imidazol-2(3H)-one (24 mg 91%) as a white solid. ¹HNMR (400 MHz, DMSO-d₆) δ 7.43-7.34 (m, 5H), 7.30 (d, J=10.8 Hz, 1H), 7.24 (br d, J=7.9 Hz, 2H), 7.01 (d, J=8.9 Hz, 2H), 5.03 (s, 2H), 3.88 (q, J=7.0 Hz, 2H), 3.27-3.20 (m, 4H), 1.49-1.43 (m, 4H), 1.23 (t, J=7.0 Hz, 3H), 0.98 (s, 6H); LCMS: 492.2 [M+H]⁺.

### Step 2: 3-(4-(4,4-Dimethylpiperidin-1-yl)phenyl)-1-ethyl-4,6-difluoro-5-hydroxy-1H-benzo[d]imidazol-2(3H)-one

5-(Benzyloxy)-3-(4-(4,4-dimethylpiperidin-1-yl)phenyl)-1-ethyl-4,6-difluoro-1H-benzo[d]imidazol-2(3H)-one (22 mg, 0.045 mmol) and THF (2 mL) was degassed with one vacuum/N₂ cycle. Palladium on carbon (5 wt. %) was added. The mixture was degassed with 3 vacuum/H₂ cycles, stirred at rtf or 1 h, and then filtered. The filter cake was washed with EtOAc (1 mL), and the filtrate was concentrated to give 3-(4-(4,4-dimethylpiperidin-1-yl)phenyl)-1-ethyl-4,6-difluoro-5-hydroxy-1H-benzo[d]imidazol-2(3H)-one (19 mg) as an off-white foam. ¹H NMR (400 MHz, DMSO-d₆) δ 9.63 (s, 1H), 7.30 (dd, J=1.5, 8.9 Hz, 2H), 7.24 (dd, J=1.4, 10.3 Hz, 1H), 7.06 (d, J=9.0 Hz, 2H), 3.91 (q, J=7.1 Hz, 2H), 3.33-3.22 (m, 4H), 1.54-1.49 (m, 4H), 1.28 (t, J=7.2 Hz, 3H), 1.04 (s, 6H); LCMS: 402.4 [M+H]⁺.

### Compound 13

### 1-(4′-Chloro-[1,1′-biphenyl]-4-yl)-5,7-difluoro-1H-indazol-6-ol

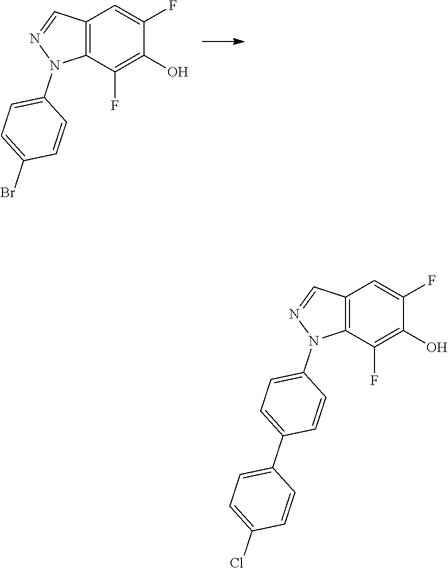 [SMILES: Oc1c(F)cc2cnn(-c3ccc(Br)cc3)c2c1F ; Oc1c(F)cc2cnn(-c3ccc(-c4ccc(Cl)cc4)cc3)c2c1F]

A mixture of Intermediate 22 (40 mg, 0.12 mmol), 4-chlorophenylboronic acid (29 mg, 0.18 mmol), 1,1′-bis(diphenylphosphino)ferrocene dichloropalladium(II) (9 mg, 0.012 mmol), 3.0 M aq. potassium carbonate (123 μL, 0.37 mmol), DME (1 mL), and water (0.35 mL) was heated at 80° C. for 1 h, allowed to cool to rt, and then diluted with EtOAc (10 mL). The organics were washed with 1.0 M HCl (5 mL), washed with brine (10 mL), dried (Na₂SO₄), filtered, concentrated, and then purified by reverse-phase HPLC (60-80% CH₃CN in water with 0.1% TFA). The product fractions were concentrated to remove the acetonitrile, and the resulting solids were filtered. The filter cake was washed with water (5 mL) to give 1-(4′-chloro-[1,1′-biphenyl]-4-yl)-5,7-difluoro-1H-indazol-6-ol (18 mg, 41%) as an off-white solid. ¹HNMR (400 MHz, DMSO-d₆): δ 10.63 (s, 1H), 8.34 (d, J=2.0 Hz, 1H), 7.86 (d, J=8.4 Hz, 2H), 7.80 (d, J=8.4 Hz, 2H), 7.71 (dd, J=3.0, 8.5 Hz, 2H), 7.57 (d, J=8.7 Hz, 3H); LCMS: 356.9 [M+H]⁺.

The Compounds below were synthesized from Intermediate 22 or Intermediate 23 and the appropriate boronic acid following the procedures described for Compound 13.

| Cmpd | Structure | Name | [M + H]⁺ |
|---|---|---|---|
| 13.1 | 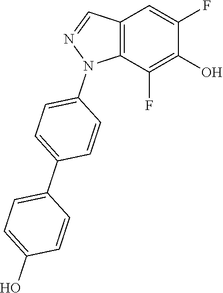 [SMILES: Oc1ccc(-c2ccc(-n3ncc4cc(F)c(O)c(F)c43)cc2)cc1] | 5,7-Difluoro-1-(4′-hydroxy-[1,1′- biphenyl]-4-yl)-1H-indazol-6-ol | 338.9 |
| 13.2 | 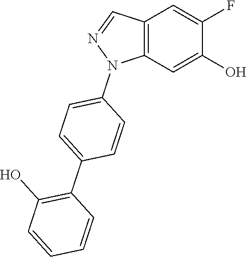 [SMILES: Oc1cc2c(cnn2-c2ccc(-c3ccccc3O)cc2)cc1F] | 5-Fluoro-1-(2′-hydroxy-[1,1′- biphenyl]-4-yl)-1H-indazol-6-ol | 320.9 |
| 13.3 | 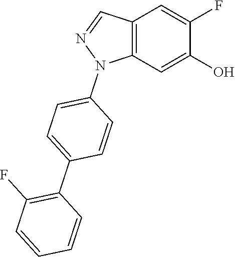 [SMILES: Oc1cc2c(cnn2-c2ccc(-c3ccccc3F)cc2)cc1F] | 5-Fluoro-1-(2′-fluoro-[1,1′- biphenyl]-4-yl)-1H-indazol-6-ol | 322.9 |
| 13.4 | 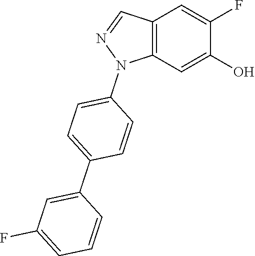 [SMILES: Oc1cc2c(cnn2-c2ccc(-c3cccc(F)c3)cc2)cc1F] | 5-Fluoro-1-(3′-fluoro-[1,1′- biphenyl]-4-yl)-1H-indazol-6-ol | 322.9 |
| 13.5 | 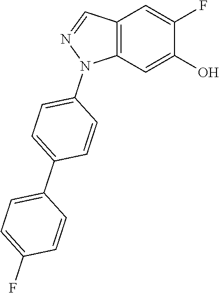 [SMILES: Oc1cc2c(cnn2-c2ccc(-c3ccc(F)cc3)cc2)cc1F] | 5-Fluoro-1-(4′-fluoro-[1,1′- biphenyl]-4-yl)-1H-indazol-6-ol | 322.9 |
| 13.6 | 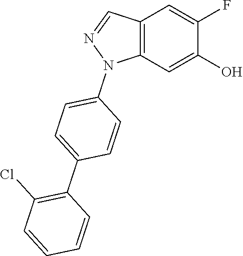 [SMILES: Oc1cc2c(cnn2-c2ccc(-c3ccccc3Cl)cc2)cc1F] | 1-(2′-Chloro-[1,1′-biphenyl]-4-yl)- 5-fluoro-1H-indazol-6-ol | 338.8 |
| 13.7 | 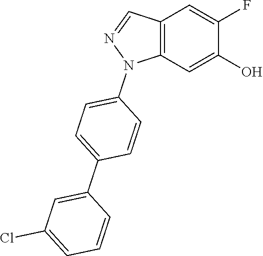 [SMILES: Oc1cc2c(cnn2-c2ccc(-c3cccc(Cl)c3)cc2)cc1F] | 1-(3′-Chloro-[1,1′-biphenyl]-4-yl)- 5-fluoro-1H-indazol-6-ol | 338.8 |
| 13.8 | 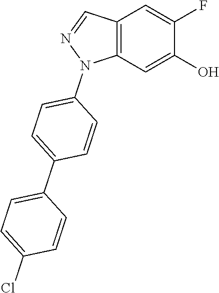 [SMILES: Oc1cc2c(cnn2-c2ccc(-c3ccc(Cl)cc3)cc2)cc1F] | 1-(4′-Chloro-[1,1′-biphenyl]-4-yl)- 5-fluoro-1H-indazol-6-ol | 338.8 |
| 13.9 | 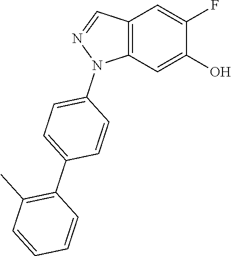 [SMILES: Cc1ccccc1-c1ccc(-n2ncc3cc(F)c(O)cc32)cc1] | 5-Fluoro-1-(2′-methyl-[1,1′- biphenyl]-4-yl)-1H-indazol-6-ol | 318.9 |
| 13.10 | 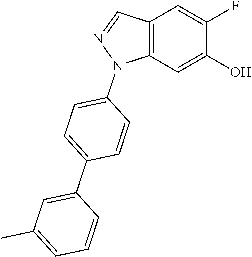 [SMILES: Cc1cccc(-c2ccc(-n3ncc4cc(F)c(O)cc43)cc2)c1] | 5-Fluoro-1-(3′-methyl-[1,1′- biphenyl]-4-yl)-1H-indazol-6-ol | 318.9 |
| 13.11 | 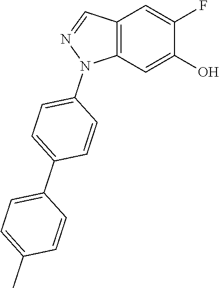 [SMILES: Cc1ccc(-c2ccc(-n3ncc4cc(F)c(O)cc43)cc2)cc1] | 5-Fluoro-1-(4′-methyl-[1,1′- biphenyl]-4-yl)-1H-indazol-6-ol | 318.9 |
| 13.12 | 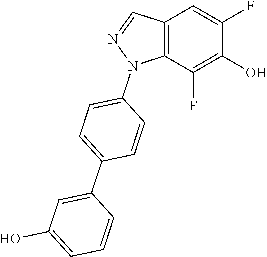 [SMILES: Oc1cccc(-c2ccc(-n3ncc4cc(F)c(O)c(F)c43)cc2)c1] | 5,7-Difluoro-1-(3′-hydroxy-[1,1′- biphenyl]-4-yl)-1H-indazol-6-ol | 338.9 |
| 13.13 | 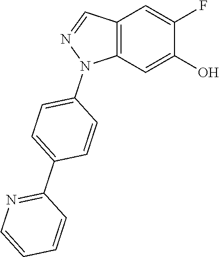 [SMILES: Oc1cc2c(cnn2-c2ccc(-c3ccccn3)cc2)cc1F] | 5-Fluoro-1-(4-(pyridin-2- yl)phenyl)-1H-indazol-6-ol | 305.9 |
| 13.14 | 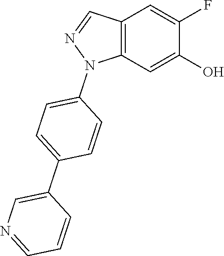 [SMILES: Oc1cc2c(cnn2-c2ccc(-c3cccnc3)cc2)cc1F] | 5-Fluoro-1-(4-(pyridin-3- yl)phenyl)-1H-indazol-6-ol | 305.9 |
| 13.15 | 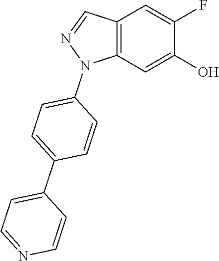 [SMILES: Oc1cc2c(cnn2-c2ccc(-c3ccncc3)cc2)cc1F] | 5-Fluoro-1-(4-(pyridin-4- yl)phenyl)-1H-indazol-6-ol | 305.9 |
| 13.16 | 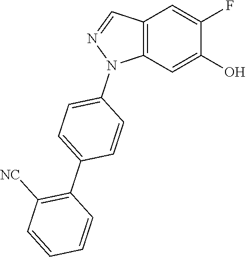 [SMILES: N#Cc1ccccc1-c1ccc(-n2ncc3cc(F)c(O)cc32)cc1] | 4′-(5-Fluoro-6-hydroxy-1H- indazol-1-yl)-[1,1′-biphenyl]-2- carbonitrile | 329.9 |
| 13.17 | 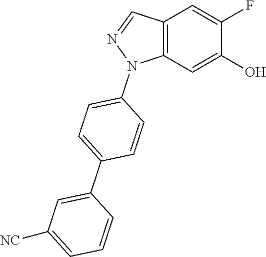 [SMILES: N#Cc1cccc(-c2ccc(-n3ncc4cc(F)c(O)cc43)cc2)c1] | 4′-(5-Fluoro-6-hydroxy-1H- indazol-1-yl)-[1,1′-biphenyl]-3- carbonitrile | 329.9 |
| 13.18 | 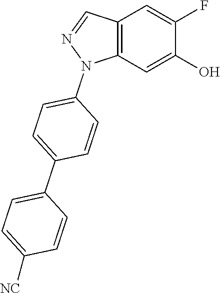 [SMILES: N#Cc1ccc(-c2ccc(-n3ncc4cc(F)c(O)cc43)cc2)cc1] | 4′-(5-Fluoro-6-hydroxy-1H- indazol-1-yl)-[1,1′-biphenyl]-4- carbonitrile | 329.9 |
| 13.19 | 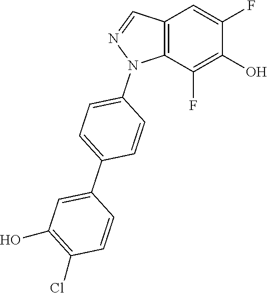 [SMILES: Oc1cc(-c2ccc(-n3ncc4cc(F)c(O)c(F)c43)cc2)ccc1Cl] | 1-(4′-Chloro-3′-hydroxy-[1,1′- biphenyl]-4-yl)-5,7-difluoro-1H- indazol-6-ol | 372.8 |
| 13.20 | 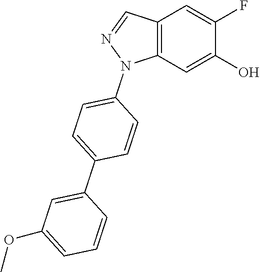 [SMILES: COc1cccc(-c2ccc(-n3ncc4cc(F)c(O)cc43)cc2)c1] | 5-Fluoro-1-(3′-methoxy-[1,1′- biphenyl]-4-yl)-1H-indazol-6-ol | 334.9 |
| 13.21 | 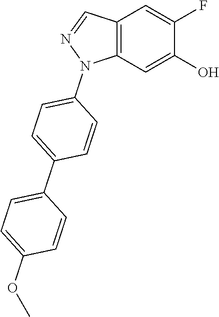 [SMILES: COc1ccc(-c2ccc(-n3ncc4cc(F)c(O)cc43)cc2)cc1] | 5-Fluoro-1-(4′-methoxy-[1,1′- biphenyl]-4-yl)-1H-indazol-6-ol | 334.9 |
| 13.22 | 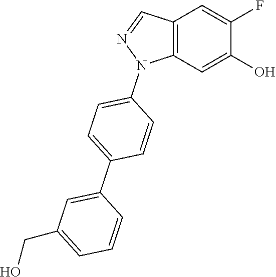 [SMILES: OCc1cccc(-c2ccc(-n3ncc4cc(F)c(O)cc43)cc2)c1] | 5-Fluoro-1-(3′-(hydroxymethyl)- [1,1′-biphenyl]-4-yl)-1H-indazol- 6-ol | 334.9 |
| 13.23 | 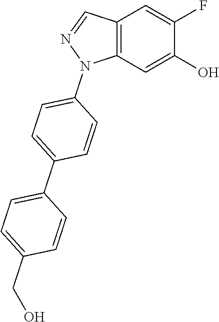 [SMILES: OCc1ccc(-c2ccc(-n3ncc4cc(F)c(O)cc43)cc2)cc1] | 5-Fluoro-1-(4′-(hydroxymethyl)- [1,1′-biphenyl]-4-yl)-1H-indazol- 6-ol | 334.9 |
| 13.24 | 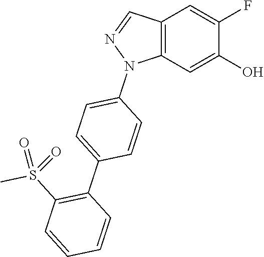 [SMILES: CS(=O)(=O)c1ccccc1-c1ccc(-n2ncc3cc(F)c(O)cc32)cc1] | 5-Fluoro-1-(2′-(methylsulfonyl)- [1,1′-biphenyl]-4-yl)-1H-indazol- 6-ol | 382.9 |
| 13.25 | 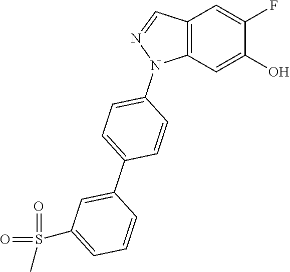 [SMILES: CS(=O)(=O)c1cccc(-c2ccc(-n3ncc4cc(F)c(O)cc43)cc2)c1] | 5-Fluoro-1-(3′-(methylsulfonyl)- [1,1′-biphenyl]-4-yl)-1H-indazol- 6-ol | 382.9 |
| 13.26 | 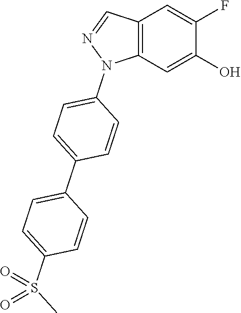 [SMILES: CS(=O)(=O)c1ccc(-c2ccc(-n3ncc4cc(F)c(O)cc43)cc2)cc1] | 5-Fluoro-1-(4′-(methylsulfonyl)- [1,1′-biphenyl]-4-yl)-1H-indazol- 6-ol | 382.9 |
| 13.27 | 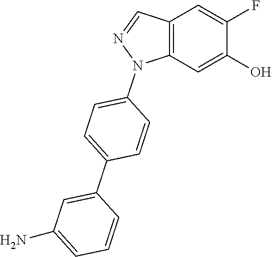 [SMILES: Nc1cccc(-c2ccc(-n3ncc4cc(F)c(O)cc43)cc2)c1] | 1-(3′-Amino-[1,1′-biphenyl]-4-yl)- 5-fluoro-1H-indazol-6-ol | 320.1 |
| 13.28 | 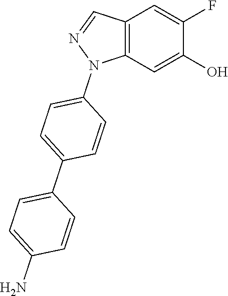 [SMILES: Nc1ccc(-c2ccc(-n3ncc4cc(F)c(O)cc43)cc2)cc1] | 1-(4′-Amino-[1,1′-biphenyl]-4-yl)- 5-fluoro-1H-indazol-6-ol | 320.1 |
| 13.29 | 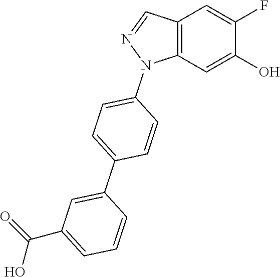 [SMILES: O=C(O)c1cccc(-c2ccc(-n3ncc4cc(F)c(O)cc43)cc2)c1] | 4′-(5-Fluoro-6-hydroxy-1H- indazol-1-yl)-[1,1′-biphenyl]-3- carboxylic acid | 348.9 |
| 13.30 | 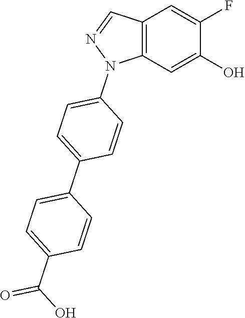 [SMILES: O=C(O)c1ccc(-c2ccc(-n3ncc4cc(F)c(O)cc43)cc2)cc1] | 4′-(5-Fluoro-6-hydroxy-1H- indazol-1-yl)-[1,1′-biphenyl]-4- carboxylic acid | 348.9 |
| 13.31 | 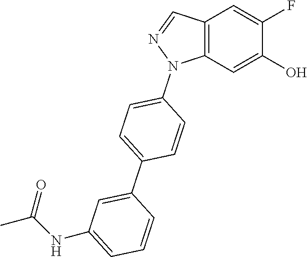 [SMILES: CC(=O)Nc1cccc(-c2ccc(-n3ncc4cc(F)c(O)cc43)cc2)c1] | N-(4′-(5-Fluoro-6-hydroxy-1H- indazol-1-yl)-[1,1′-biphenyl]-3- yl)acetamide | 361.9 |
| 13.32 | 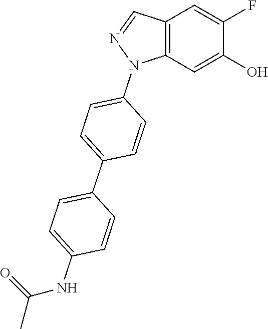 [SMILES: CC(=O)Nc1ccc(-c2ccc(-n3ncc4cc(F)c(O)cc43)cc2)cc1] | N-(4′-(5-Fluoro-6-hydroxy-1H- indazol-1-yl)-[1,1′-biphenyl]-4- yl)acetamide | 361.9 |
| 13.33 | 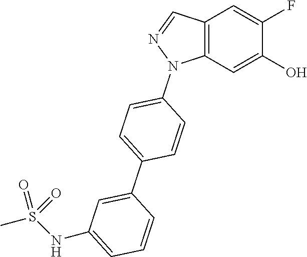 [SMILES: CS(=O)(=O)Nc1cccc(-c2ccc(-n3ncc4cc(F)c(O)cc43)cc2)c1] | N-(4′-(5-Fluoro-6-hydroxy-1H- indazol-1-yl)-[1,1′-biphenyl]-3- yl)methanesulfonamide | 397.9 |
| 13.34 | 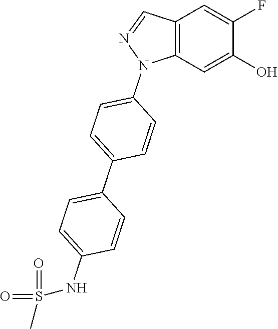 [SMILES: CS(=O)(=O)Nc1ccc(-c2ccc(-n3ncc4cc(F)c(O)cc43)cc2)cc1] | N-(4′-(5-Fluoro-6-hydroxy-1H- indazol-1-yl)-[1,1′-biphenyl]-4- yl)methanesulfonamide | 398.0 |
| 13.35 | 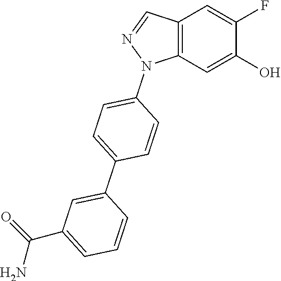 [SMILES: NC(=O)c1cccc(-c2ccc(-n3ncc4cc(F)c(O)cc43)cc2)c1] | 4′-(5-Fluoro-6-hydroxy-1H- indazol-1-yl)-[1,1′-biphenyl]-3- carboxamide | 347.9 |
| 13.36 | 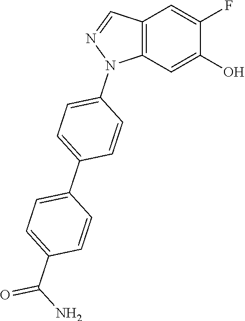 [SMILES: NC(=O)c1ccc(-c2ccc(-n3ncc4cc(F)c(O)cc43)cc2)cc1] | 4′-(5-Fluoro-6-hydroxy-1H- indazol-1-yl)-[1,1′-biphenyl]-4- carboxamide | 347.9 |
| 13.37 | 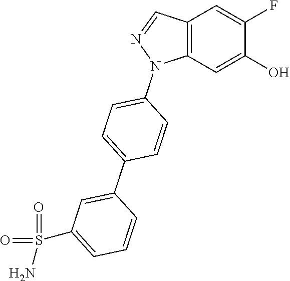 [SMILES: NS(=O)(=O)c1cccc(-c2ccc(-n3ncc4cc(F)c(O)cc43)cc2)c1] | 4′-(5-Fluoro-6-hydroxy-1H- indazol-1-yl)-[1,1′-biphenyl]-3- sulfonamide | 383.9 |
| 13.38 | 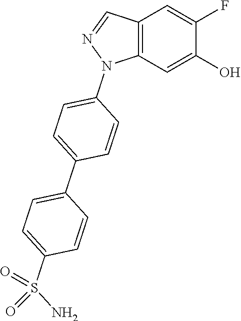 [SMILES: NS(=O)(=O)c1ccc(-c2ccc(-n3ncc4cc(F)c(O)cc43)cc2)cc1] | 4′-(5-Fluoro-6-hydroxy-1H- indazol-1-yl)-[1,1′-biphenyl]-4- sulfonamide | 383.9 |
| 13.39 | 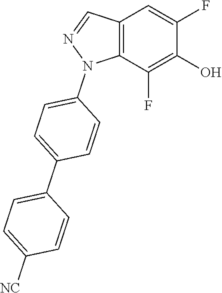 [SMILES: N#Cc1ccc(-c2ccc(-n3ncc4cc(F)c(O)c(F)c43)cc2)cc1] | 4′-(5,7-Difluoro-6-hydroxy-1H- indazol-1-yl)-[1,1′-biphenyl]-4- carbonitrile | 347.9 |
| 13.40 | 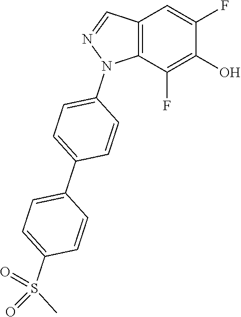 [SMILES: CS(=O)(=O)c1ccc(-c2ccc(-n3ncc4cc(F)c(O)c(F)c43)cc2)cc1] | 5,7-Difluoro-1-(4′- (methylsulfonyl)-[1,1′-biphenyl]- 4-yl)-1H-indazol-6-ol | 400.9 |

Alternate conditions: 1-65 h. Also, microwave, 100° C., 20 min. In some instances, additional boronic acid, Pd(dppf)Cl₂, and K₂CO₃ were needed for full conversion.

### Compound 14

### 5-(4-(5,7-Difluoro-6-hydroxy-LH-indazol-1-yl)phenyl)picolinonitrile

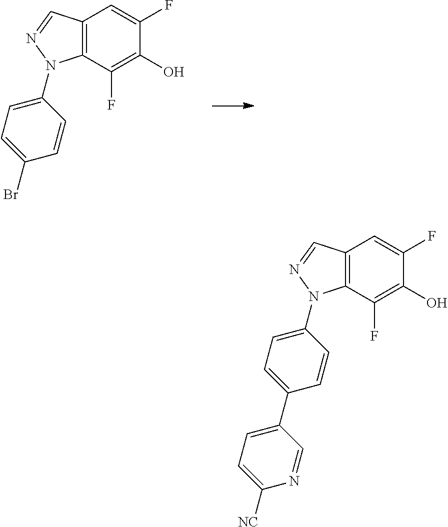 [SMILES: Oc1c(F)cc2cnn(-c3ccc(Br)cc3)c2c1F ; N#Cc1ccc(-c2ccc(-n3ncc4cc(F)c(O)c(F)c43)cc2)cn1]

Intermediate 22 (50 mg, 0.15 mmol), 2-cyanopyridine-5-boronic acid (34 mg, 0.23 mmol), cesium carbonate (200 mg, 0.61 mmol), bis(triphenylphosphine)palladium(II)chloride (12 mg, 0.02 mmol), DMF (2 mL), and then water (1 mL) were combined in an 8 mL vial.

The reaction was degassed with 3 vacuum/N₂ cycles, stirred at 100° C. for 1 h, allowed to cool to rt, and then diluted with 20 mL EtOAc and 20 mL water. The organic layer was washed with 20 mL brine, dried (Na₂SO₄), filtered, concentrated, and then purified by reverse-phase HPLC (49.1-59.1% CH₃CN in water with 0.1% TFA). The pure fractions were concentrated, and the residue was diluted with 20 mL EtOAc and 20 mL saturated NaHCO₃. The organic layer was washed with 20 mL saturated NaHCO₃, washed with 20 mL brine, dried (Na₂SO₄), filtered, and then concentrated to give 5-(4-(5,7-difluoro-6-hydroxy-H-indazol-1-yl)phenyl)picolinonitrile (9.2 mg, 17%) as an off-white solid. ¹H NMR (400 MHz, DMSO-d₆): δ 10.66 (s, 1H), 9.21 (s, 1H), 8.46 (br d, J=8.3 Hz, 1H), 8.36 (s, 1H), 8.17 (d, J=8.1 Hz, 1H), 8.03 (d, J=8.3 Hz, 2H), 7.79 (dd, J=2.5, 8.4 Hz, 2H), 7.57 (d, J=9.7 Hz, 1H); LCMS: 348.9 [M+H]⁺.

The Compounds below were synthesized from Intermediate 22 and the appropriate boronic acid following the procedure described for Compound 14.

| Cmpd | Structure | Name | [M + H]⁺ |
|---|---|---|---|
| 14.1¹ | 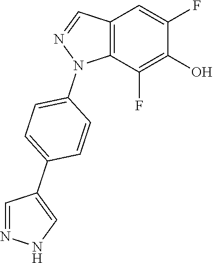 [SMILES: Oc1c(F)cc2cnn(-c3ccc(-c4cn[nH]c4)cc3)c2c1F] | 1-(4-(1H-Pyrazol-4-yl)phenyl)- 5,7-difluoro-1H-indazol-6-ol | 312.9 |
| 14.2¹ | 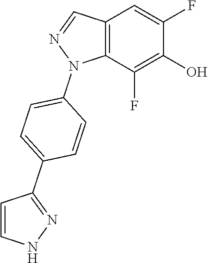 [SMILES: Oc1c(F)cc2cnn(-c3ccc(-c4cc[nH]n4)cc3)c2c1F] | 1-(4-(1H-Pyrazol-3-yl)phenyl)- 5,7-difluoro-1H-indazol-6-ol | 313.0 |
| 14.3² | 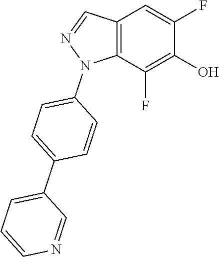 [SMILES: Oc1c(F)cc2cnn(-c3ccc(-c4cccnc4)cc3)c2c1F] | 5,7-Difluoro-1-(4-(pyridin-3- yl)phenyl)-1H-indazol-6-ol | 323.9 |
| 14.4³ | 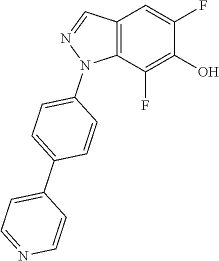 [SMILES: Oc1c(F)cc2cnn(-c3ccc(-c4ccncc4)cc3)c2c1F] | 5,7-Difluoro-1-(4-(pyridin-4- yl)phenyl)-1H-indazol-6-ol | 323.9 |

Alternate conditions:
¹Na₂CO₃ (aq), Pd(PPh₃)₄, DMF, 100° C., 1.3 h.
²K₃PO₄ (aq), Pd₂(dba)₃, P(Cy)₃, dioxane, 100° C., 3.5 h.
³Triturated in CH₂Cl₂ instead of HPLC purification.

### Compound 15

### 1-(3′-Chloro-4′-hydroxy-[1,1′-biphenyl]-4-yl)-5-fluoro-1H-indazol-6-ol

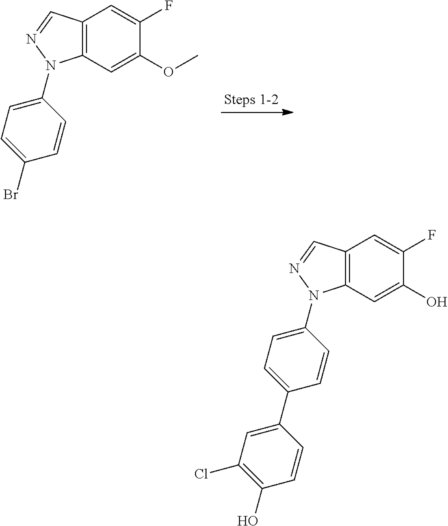 [SMILES: COc1cc2c(cnn2-c2ccc(Br)cc2)cc1F ; Oc1cc2c(cnn2-c2ccc(-c3ccc(O)c(Cl)c3)cc2)cc1F]

### Step 1: 3-Chloro-4′-(5-fluoro-6-methoxy-LH-indazol-1-yl)-[1,1′-biphenyl]-4-ol

A mixture of Intermediate 13.6 (70 mg, 0.22 mmol), 3-chloro-4-hydroxyphenylboronic acid (56 mg, 0.33 mmol), tetrakis(triphenylphosphine)palladium (13 mg, 0.01 mmol), cesium carbonate (284 mg, 0.87 mmol), DME (2 mL), and water (1 mL) was heated at 100° C. for 30 min. The aqueous layer was pipetted off, and the remaining mixture was diluted with EtOAc (10 mL). The organic layer was washed with brine (10 mL), dried (Na₂SO₄), filtered, and then concentrated. The crude solid was suspended in CH₃OH (3 mL) and minimal CH₂Cl₂ (0.2 mL), and the mixture was sonicated for 1 min. The solids were filtered, and the filter cake was washed with CH₃OH (2×2 mL) to give 3-chloro-4′-(5-fluoro-6-methoxy-1H-indazol-1-yl)-[1,1′-biphenyl]-4-ol (67 mg, 83%) as an off-white solid. 1H NMR (400 MHz, DMSO-d₆): δ 10.40 (s, 1H), 8.26 (s, 1H), 7.84 (s, 4H), 7.76-7.69 (m, 2H), 7.57 (dd, J=2.3, 8.4 Hz, 1H), 7.39 (d, J=7.0 Hz, 1H), 7.09 (d, J=8.6 Hz, 1H), 3.97 (s, 3H); LCMS: 368.9 [M+H]⁺.

### Step 2: 1-(3′-Chloro-4′-hydroxy-[1,1′-biphenyl]-4-yl)-5-fluoro-1H-indazol-6-ol

Boron tribromide (1 M in CH₂Cl₂, 0.49 mL, 0.49 mmol) was added slowly to a mixture of 3-chloro-4′-(5-fluoro-6-methoxy-1H-indazol-1-yl)-[1,1′-biphenyl]-4-ol (60 mg, 0.16 mmol) in CH₂Cl₂ (3 mL) at −78° C. under N₂. The mixture was allowed to warm to rt, stirred for 3 h, and then cooled in a dry ice/acetone bath. Boron tribromide (1 M in CH₂Cl₂, 0.33 mL, 0.33 mmol) was added slowly. The mixture was allowed to warm to rt, stirred for 2 h, and then cooled in a dry ice/acetone bath. Boron tribromide (1 M in CH₂Cl₂, 0.81 mL, 0.81 mmol) was added slowly. The mixture was allowed to warm to rt, stirred for 17 h, and then cooled in a dry ice/acetone bath. Boron tribromide (1 M in CH₂Cl₂, 1.62 mL, 1.62 mmol) was added slowly. The mixture was allowed to warm to rt, stirred for 7 h, and then heated at 35° C. for 40 h. The mixture was cooled in a dry ice/acetone bath, quenched with CH₃OH (5 mL), and concentrated. The crude solids were suspended in 2:1 EtOAc/CH₂Cl₂ (3 mL), stirred at rt for 1 h, filtered, and then purified by silica gel chromatography (0-5% CH₃OH in CH₂Cl₂) to give 1-(3′-chloro-4′-hydroxy-[1,1′-biphenyl]-4-yl)-5-fluoro-1H-indazol-6-ol (37 mg, 600%) as a beige solid. ¹H NMR (400 MHz, DMSO-d₆): δ 10.42 (br s, 2H), 8.21 (s, 1H), 7.85-7.81 (m, 2H), 7.77-7.72 (m, 3H), 7.64 (d, J=10.6 Hz, 1H), 7.56 (dd, J=2.3, 8.4 Hz, 1H), 7.31 (d, J=7.2 Hz, 1H), 7.09 (d, J=8.4 Hz, 1H); LCMS: 354.9 [M+H]⁺.

The Compounds below were synthesized from the appropriate Intermediate and the appropriate boronic acid following the procedures described for Compound 15.

| Cmpd | Structure | Name | [M + H]⁺ |
|---|---|---|---|
| 15.1 | 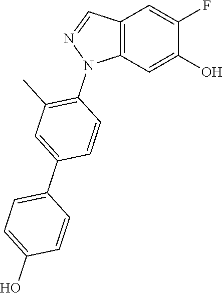 [SMILES: Cc1cc(-c2ccc(O)cc2)ccc1-n1ncc2cc(F)c(O)cc21] | 5-Fluoro-1-(4′-hydroxy-3-methyl- [1,1′-biphenyl]-4-yl)-1H-indazol- 6-ol | 335.0 |
| 15.2 | 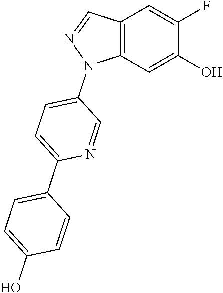 [SMILES: Oc1ccc(-c2ccc(-n3ncc4cc(F)c(O)cc43)cn2)cc1] | 5-Fluoro-1-(6-(4- hydroxyphenyl)pyridin-3-yl)-1H- indazol-6-ol | 321.9 |
| 15.3¹ | 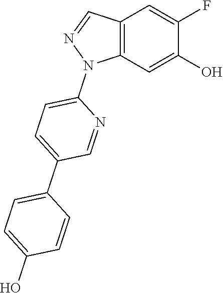 [SMILES: Oc1ccc(-c2ccc(-n3ncc4cc(F)c(O)cc43)nc2)cc1] | 5-Fluoro-1-(5-(4- hydroxyphenyl)pyridin-2-yl)-1H- indazol-6-ol | 321.9 |
| 15.4² | 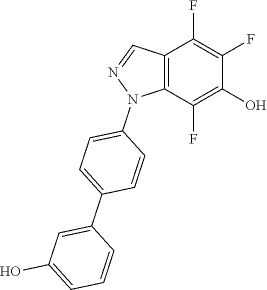 [SMILES: Oc1cccc(-c2ccc(-n3ncc4c(F)c(F)c(O)c(F)c43)cc2)c1] | 4,5,7-Trifluoro-1-(3′-hydroxy- [1,1′-biphenyl]-4-yl)-1H-indazol- 6-ol | 356.9 |

Alternate conditions: Step 1: DME/EtOH (2:1) as solvent; 80-100° C.; 0.5-2 h. Step 2: 5-10 eq BBr₃ was usually sufficient for full conversion; 35-40° C., 17-19h.
¹Pyridine HCl, 170° C., 6 h.
²Step 2 only from Intermediate 14.7.

### Compound 16

### 4,7-Difluoro-1-(3′-hydroxy-[1,1′-biphenyl]-4-yl)-1H-indazol-6-ol

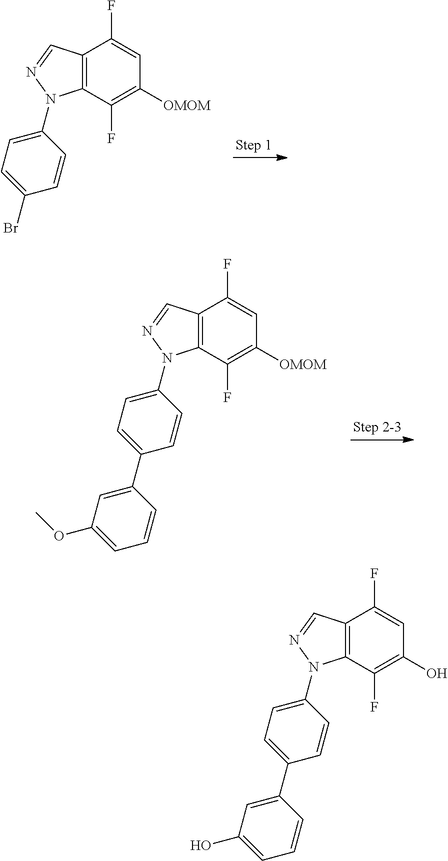

### Step 1: 4,7-Difluoro-1-(3′-methoxy-[1,1′-biphenyl]-4-yl)-6-(methoxymethoxy)-1H-indazole

A mixture of Intermediate 13 (85 mg, 0.23 mmol), (3-methoxyphenyl)boronic acid (52.5 mg, 0.345 mmol), Pd(dppf)Cl₂ (16.8 mg, 0.23 mmol), KOAc (67.8 mg, 0.690 mmol), and dioxane (5 mL) was stirred at 110° C. overnight under N₂. The mixture was allowed to cool to rt, poured into water (10 mL), and then extracted with EtOAc (2×15 mL). The combined organic layers were washed with water (2×10 mL), washed with brine (10 mL), dried (Na₂SO₄), filtered, concentrated, and then purified by prep-TLC (petroleum ether/EtOAc=1/1) to give 4,7-difluoro-1-(3′-methoxy-[1,1′-biphenyl]-4-yl)-6-(methoxymethoxy)-1H-indazole (80 mg, 88%) as a yellow solid. ¹H NMR (400 MHz, DMSO-d₆): δ 8.53 (d, 1H), 7.87 (d, 2H), 7.78-7.68 (m, 2H), 7.47-7.40 (m, 1H), 7.35-7.27 (m, 2H), 7.24-7.14 (m, 1H), 6.99 (d, 1H), 5.33 (s, 2H), 3.86 (s, 3H), 3.45 (s, 3H); LCMS: 397.1 [M+H]⁺.

### Step 2: 4,7-Difluoro-1-(3′-methoxy-[1,1′-biphenyl]-4-yl)-1H-indazol-6-ol

Trifluoroacetic acid (4.62 g, 40.5 mmol) was added to a solution of 4,7-difluoro-1-(3′-methoxy-[1,1′-biphenyl]-4-yl)-6-(methoxymethoxy)-1H-indazole (60 mg, 0.15 mmol) in CH₂Cl₂ (10 mL) at rt. The mixture was stirred for 2 h, poured into saturated NaHCO₃ (10 mL), and then extracted with EtOAc (2×20 mL). The combined organic layers were washed with water (2×10 mL), washed with brine (10 mL), dried (Na₂SO₄), filtered, and then concentrated to give 4,7-difluoro-1-(3′-methoxy-[1,1′-biphenyl]-4-yl)-1H-indazol-6-ol (54 mg) as a yellow oil. LCMS: 353.0 [M+H]⁺.

### Step 3: 4,7-Difluoro-1-(3′-hydroxy-[1,1′-biphenyl]-4-yl)-1H-indazol-6-ol

Boron tribromide (1.09 g, 4.35 mmol) was added to a solution of 4,7-difluoro-1-(3′-methoxy-[1,1′-biphenyl]-4-yl)-1H-indazol-6-ol (54 mg, 0.15 mmol) in CH₂Cl₂ (5 mL) at −78° C. The mixture was allowed to warm to 0° C. for 1 h and then warm to rt overnight. The mixture was poured into CH₃OH (10 mL) carefully, adjusted to pH=˜7 with saturated NaHCO₃, and then extracted with EtOAc (2×20 mL). The combined organic layers were washed with water (10 mL×2), washed with brine (10 mL), dried (Na₂SO₄), filtered, concentrated, and then purified by reverse-phase HPLC [water(0.04% HCl)—CH₃CN] to give 4,7-difluoro-1-(3′-hydroxy-[1,1′-biphenyl]-4-yl)-1H-indazol-6-ol (9.9 mg, 19%) as a yellow solid. ¹HNMR (400 MHz, DMSO-d₆): δ 10.59 (s, 1H), 9.60 (s, 1H), 8.42 (s, 1H), 7.77 (d, 2H), 7.72-7.64 (m, 2H), 7.35-7.26 (m, 1H), 7.16 (d, 1H), 7.19-7.10 (m, 1H), 6.86-6.73 (m, 2H); LCMS: 339.1 [M+H]⁺.

### Compound 17

### 7-Chloro-5-fluoro-1-(3′-hydroxy-[1,1′-biphenyl]-4-yl)-1H-indazol-6-ol

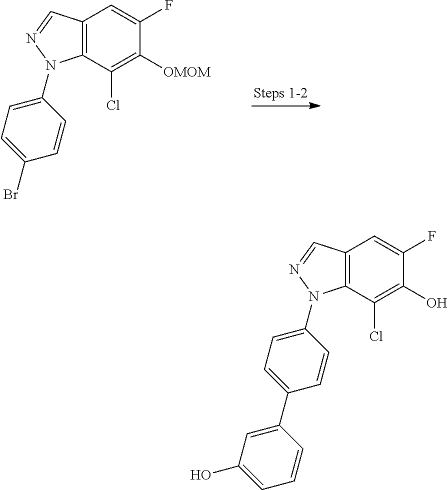

### Step 1: 7-Chloro-5-fluoro-1-(3′-methoxy-[1,1′-biphenyl]-4-yl)-6-(methoxymethoxy)-1H-indazole

7-Chloro-5-fluoro-1-(3′-methoxy-[1,1′-biphenyl]-4-yl)-6-(methoxymethoxy)-1H-indazole (200 mg, crude) was synthesized from Intermediate 13.1 and (3-methoxyphenyl)boronic acid following the procedure described for Compound 16, Step 1. ¹H NMR (400 MHz, DMSO-d₆): δ 9.25-8.40 (m, 1H), 8.17-7.90 (m, 2H), 7.86-7.59 (m, 2H), 7.83-7.68 (m, 1H), 7.44-7.40 (m, 1H), 7.34-7.28 (m, 2H), 7.00-6.97 (m, 1H), 5.26-5.22 (m, 2H), 3.85 (s, 3H), 3.55 (s, 3H); LCMS: 413.2 [M+H]⁺.

### Step 2: 7-Chloro-5-fluoro-1-(3′-hydroxy-[1,1′-biphenyl]-4-yl)-1H-indazol-6-ol

Boron tribromide (1.21 g, 4.84 mmol) was added dropwise to a solution of 7-chloro-5-fluoro-1-(3′-methoxy-[1,1′-biphenyl]-4-yl)-6-(methoxymethoxy)-1H-indazole (200 mg) in CH₂Cl₂ (2 mL) at −78° C. The mixture was stirred for 1 h, stirred at rt for 2 h, and then added dropwise into CH₃OH (15 mL) at 0° C. The mixture was adjusted to pH˜7-8 with saturated NaHCO₃ (˜20 mL) and extracted with CH₂Cl₂ (3×20 mL). The combined organic layers were washed with brine (30 mL), dried over Na₂SO₄, filtered, and then concentrated. The residue was dissolved in CH₂Cl₂ (10 mL). Trifluoroacetic acid (2 mL) was added. The reaction was stirred at rt for 1 h, concentrated, and then purified by prep-HPLC [water (0.04% HCl)—CH₃CN]. The material was purified further by prep-HPLC [water(10 mM NH₄HCO₃)—CH₃CN] to give 7-chloro-5-fluoro-1-(3′-hydroxy-[1,1′-biphenyl]-4-yl)-1H-indazol-6-ol (12.0 mg, 7%) as a white solid. ¹H NMR (400 MHz, DMSO-d₆): δ 10.48 (s, 1H), 9.61 (s, 1H), 8.26 (s, 1H), 7.74-7.72 (m, 2H), 7.68 (d, 1H), 7.55-7.52 (m, 2H), 7.31-7.27 (m, 1H), 7.17-7.15 (m, 1H), 7.11-7.10 (m, 1H), 6.80 (d, 1H); LCMS: 355.1 [M+H]⁺.

### Compound 18

### 1-(5-(4-Chlorophenyl)pyrazin-2-yl)-5,7-difluoro-1H-indazol-6-ol

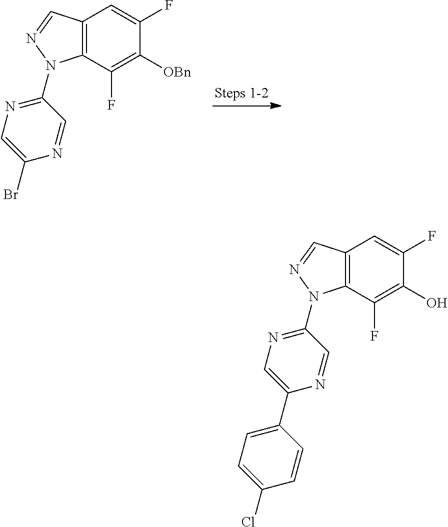 [SMILES: Fc1cc2cnn(-c3cnc(Br)cn3)c2c(F)c1OCc1ccccc1 ; Oc1c(F)cc2cnn(-c3cnc(-c4ccc(Cl)cc4)cn3)c2c1F]

### Step 1: 6-(Benzyloxy)-1-(5-(4-chlorophenyl)pyrazin-2-yl)-5,7-difluoro-1H-indazole

Pd(dppf)Cl₂·CH₂Cl₂ (11.7 mg, 0.014 mmol) was added to a solution of Intermediate 26 (100 mg, 0.240 mmol), (4-chlorophenyl)boronic acid (53.9 mg, 0.345 mmol), and Na₂CO₃ (2 M, 0.7 mL) in dioxane (3 mL) under N₂. The mixture was degassed with 3 vacuum/N₂ cycles, stirred at 80° C. for 3 h, cooled to rt, and then filtered through Celite. The filtrate was poured into H₂O (5 mL) and extracted with EtOAc (3×5 mL). The combined organic layers were washed with brine (10 mL), dried over Na₂SO₄, filtered, concentrated, and then purified by silica gel chromatography (petroleum ether/ethyl acetate=20/1 to 1/1) to give 6-(benzyloxy)-1-(5-(4-chlorophenyl)pyrazin-2-yl)-5,7-difluoro-1H-indazole (70 mg, 65%) as a white solid. ¹HNMR (400 MHz, DMSO-d₆): δ 9.28 (d, 1H), 9.19 (s, 1H), 8.58 (d, 1H), 8.25-8.23 (m, 2H), 7.72 (d, 1H), 7.64-7.62 (m, 2H), 7.51-7.44 (m, 2H), 7.44-7.33 (m, 3H), 5.26 (s, 2H); LCMS: 449.2 [M+H]⁺.

### Step 2: 1-(5-(4-Chlorophenyl)pyrazin-2-yl)-5,7-difluoro-1H-indazol-6-ol

Boron tribromide (0.35 g, 1.40 mmol) was added to a solution of 6-(benzyloxy)-1-(5-(4-chlorophenyl)pyrazin-2-yl)-5,7-difluoro-1H-indazole (70 mg, 0.155 mmol) in CH₂Cl₂ (3 mL). The mixture was stirred at −78° C. for 1 h, allowed to warm to rt, stirred at rt for 2 h, and then added into CH₃OH (5 mL) slowly at 0° C. The pH was adjusted to pH˜7-8 using saturated NaHCO₃ (˜10 mL). The mixture was extracted with CH₂Cl₂ (3×20 mL). The combined organic layers were washed with brine (20 mL), dried over Na₂SO₄, filtered, concentrated, and then purified by prep-HPLC [water(0.04% HCl)—CH₃CN] to give 1-(5-(4-chlorophenyl)pyrazin-2-yl)-5,7-difluoro-1H-indazol-6-ol (26.5 mg, 46%) as a yellow solid. ¹H NMR (400 MHz, DMSO-d₆): δ 10.76 (s, 1H), 9.27-9.22 (m, 1H), 9.21-9.17 (m, 1H), 8.48 (d, 1H), 8.24-8.22 (m, 2H), 7.64-7.62 (m, 3H); LCMS: 359.0 [M+H]⁺.

The Compounds below were synthesized from the appropriate Intermediate and the appropriate boronic acid following the procedures described for Compound 18.

| Cmpd | Structure | Name | [M + H]⁺ |
|---|---|---|---|
| 18.1^{1,3} | 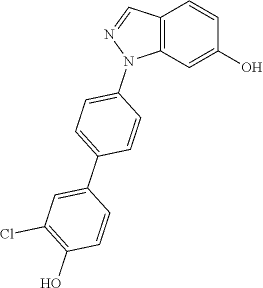 [SMILES: Oc1ccc2cnn(-c3ccc(-c4ccc(O)c(Cl)c4)cc3)c2c1] | 1-(3′-Chloro-4′-hydroxy-[1,1′- biphenyl]-4-yl)-1H-indazol-6-ol | 336.9 |
| 18.2⁴ | 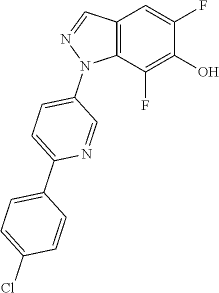 | 1-(6-(4-Chlorophenyl)pyridin-3- yl)-5,7-difluoro-1H-indazol-6-ol | 357.9 |
| 18.3² | 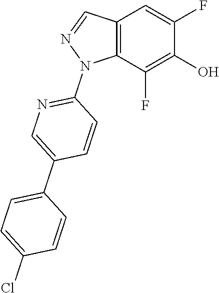 | 1-(5-(4-Chlorophenyl)pyridin-2- yl)-5,7-difluoro-1H-indazol-6-ol | 357.9 |
| 18.4 | 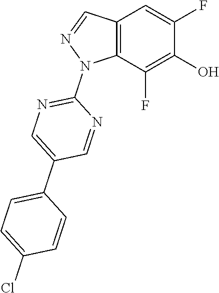 | 1-(5-(4-Chlorophenyl)pyrimidin- 2-yl)-5,7-difluoro-1H-indazol-6-ol | 359.0 |

Alternate conditions:
Step 1:
¹K₂CO₃, Pd(dppf)Cl₂, DME/water (2:1), 80° C., 3.5 h;
²Pd₂(dba)₃, XPhos, Cs₂CO₃, dioxane, H₂O, 80° C., ON.
Step 2:
³Pd/C, MeOH/EtOAc (1:1), H₂, rt, 17 h;
⁴PtO₂, MeOH, H₂, rt, 1 h.

### Compound 19

### 5,7-Difluoro-1-(4-(4-(methylsulfonyl)piperazin-1-yl)phenyl)-1H-indazol-6-ol

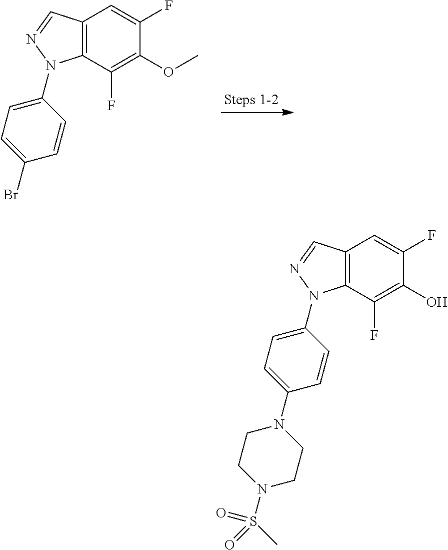 [SMILES: COc1c(F)cc2cnn(-c3ccc(Br)cc3)c2c1F ; CS(=O)(=O)N1CCN(c2ccc(-n3ncc4cc(F)c(O)c(F)c43)cc2)CC1]

### Step 1: 5,7-Difluoro-6-methoxy-1-(4-(4-(methylsulfonyl)piperazin-1-yl)phenyl)-1H-indazole

Pd₂(dba)₃ (13.5 mg, 0.014 mmol) was added to a mixture of Intermediate 14 (100 mg, 0.294 mmol), 1-methylsulfonylpiperazine (96.8 mg, 0.589 mmol), 2,2′-bis(diphenylphosphino)-1,1′-binaphthalene (14.69 mg, 0.023 mmol), Cs₂CO₃ (192 mg, 0.589 mmol), and toluene (5 mL) at rt under N₂. The mixture was degassed with 3 vacuum/N₂ cycles, stirred at 100° C. overnight, allowed to cool to rt, poured into H₂O (20 mL), and then extracted with ethyl acetate (3×20 mL). The combined organic layers were washed with brine (30 mL), dried over Na₂SO₄, filtered, concentrated, and then purified by silica gel chromatography (petroleum ether/ethyl acetate=0:40) to give 5,7-difluoro-6-methoxy-1-(4-(4-(methylsulfonyl)piperazin-1-yl)phenyl)-1H-indazole (100 mg, 80%) as a yellow solid. ¹H NMR (400 MHz, DMSO-d₆): δ 8.34-8.30 (m, 1H), 7.65-7.59 (m, 1H), 7.50-7.44 (m, 2H), 7.15-7.08 (m, 2H), 3.96 (s, 3H), 3.40-3.34 (m, 4H), 3.30-3.24 (m, 4H), 2.95 (s, 3H); LCMS: 423.2 [M+H]⁺.

### Step 2: 5,7-Difluoro-1-(4-(4-(methylsulfonyl)piperazin-1-yl)phenyl)-1H-indazol-6-ol

Boron tribromide (120 μL, 1.18 mmol) was added slowly to a mixture of 5,7-difluoro-6-methoxy-1-(4-(4-(methylsulfonyl)piperazin-1-yl)phenyl)-1H-indazole (100 mg, 0.23 mmol) in CH₂Cl₂ (5 mL) at −78° C. under N₂. The mixture was allowed to warm to rt, stirred for 2 h, and then quenched slowly with CH₃OH (10 mL). The mixture was stirred for 0.5 h, diluted with saturated NaHCO₃ (30 mL), and then extracted with ethyl acetate (3×30 mL). The combined organic layers were washed with brine (50 mL), dried over Na₂SO₄, filtered, concentrated, and then purified by reverse-phase HPLC [water (0.04% HCl)/CH₃CN] to give 5,7-difluoro-1-(4-(4-(methylsulfonyl)piperazin-1-yl)phenyl)-1H-indazol-6-ol (41 mg, 42%) as a white solid. ¹H NMR (400 MHz, DMSO-d₆): δ 10.46 (s, 1H), 8.22 (s, 1H), 7.53-7.47 (m, 1H), 7.46-7.40 (m, 2H), 7.14-7.08 (m, 2H), 3.37-3.31 (m, 4H), 3.31-3.24 (m, 4H), 2.94 (s, 3H); LCMS: 409.1 [M+H]⁺.

The Compound below was synthesized from Intermediate 14.6 and 1-methylsulfonylpiperazine following the procedures described for Compound 19.

| Cmpd | Structure | Name | [M + H]⁺ |
|---|---|---|---|
| 19.1 | 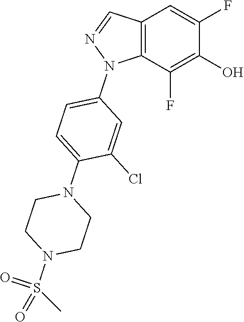 [SMILES: CS(=O)(=O)N1CCN(c2ccc(-n3ncc4cc(F)c(O)c(F)c43)cc2Cl)CC1] | 1-(3-Chloro-4-(4- (methylsulfonyl)piperazin-1- yl)phenyl)-5,7-difluoro-1H- indazol-6-ol | 443.1 |

### Compound 20

### 1-(4-(trans-2,6-Dimethylmorpholino)phenyl)-5,7-difluoro-1H-indazol-6-ol

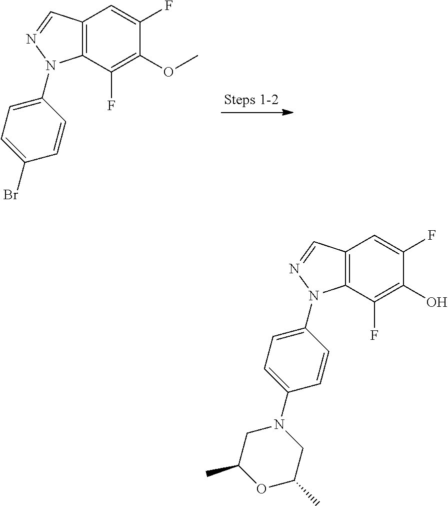 [SMILES: COc1c(F)cc2cnn(-c3ccc(Br)cc3)c2c1F ; C[C@H]1CN(c2ccc(-n3ncc4cc(F)c(O)c(F)c43)cc2)C[C@H](C)O1]

### Step 1: trans-4-(4-(5,7-Difluoro-6-methoxy-1H-indazol-1-yl)phenyl)-2,6-dimethylmorpholine

Intermediate 14.2 (50 mg, 0.15 mmol), sodium tert-butoxide* (57 mg, 0.59 mmol), trans-2,6-dimethylmorpholine (41 mg, 0.36 mmol), and toluene (1 mL) were degassed with 3 vacuum/N₂ cycles. RuPhos (7.8 mg, 0.017 mmol) and then tris(dibenzylideneacetone)dipalladium(0) (7.5 mg, 0.0082 mmol) were added. The mixture was stirred at 100° C. for 1 h, allowed to cool to rt, and then poured into a mixture of 20 mL EtOAc and 20 mL water. The organic layer was separated, washed with 20 mL brine, dried (Na₂SO₄), filtered, concentrated, and then purified by silica gel chromatography (0-25% EtOAc in hexanes) to give trans-4-(4-(5,7-difluoro-6-methoxy-1H-indazol-1-yl)phenyl)-2,6-dimethylmorpholine (50 mg, 91%) as a sticky pale yellow foam. ¹H NMR (400 MHz, DMSO-d₆): δ 8.31 (s, 1H), 7.61 (d, J=10.0 Hz, 1H), 7.44 (br d, J=6.8 Hz, 2H), 7.04 (d, J=8.8 Hz, 2H), 4.13-4.04 (m, 2H), 3.96 (s, 3H), 3.28 (dd, J=2.6, 11.8 Hz, 2H), 2.95 (dd, J=6.1, 12.0 Hz, 2H), 1.22 (d, J=6.4 Hz, 6H); LCMS: 374.2 [M+H]⁺.

* Sodium tert-butoxide was dried under high vacuum by heating with heat gun for a few minutes and then allowing to cool prior to weighing.

### Step 2: 1-(4-(trans-2,6-Dimethylmorpholino)phenyl)-5,7-difluoro-1H-indazol-6-ol

A mixture of trans-4-(4-(5,7-difluoro-6-methoxy-1H-indazol-1-yl)phenyl)-2,6-dimethylmorpholine (47 mg, 0.13 mmol) and pyridine hydrochloride (264 mg, 2.28 mmol) was stirred at 150° C. for 1.5 h, stirred at 160° C. for 2 h, stirred at 170° C. for 30 min, cooled to 100° C., diluted with 1.0 M HCl (2 mL), stirred for 5 min, allowed to cool to rt, and then poured into a mixture of 20 mL EtOAc, 20 mL water, and 20 mL saturated NaHCO₃. The organic layer was separated, washed with 20 mL brine, dried (Na₂SO₄), filtered, and then concentrated to give 1-(4-(trans-2,6-dim ethylmorpholino)phenyl)-5,7-difluoro-1H-indazol-6-ol (20 mg, 44%) as a pale yellow solid. ¹H NMR (400 MHz, DMSO-d₆): δ 10.45 (s, 1H), 8.21 (s, 1H), 7.49 (d, J=9.8 Hz, 1H), 7.40 (br d, J=7.1 Hz, 2H), 7.03 (d, J=8.8 Hz, 2H), 4.15-4.03 (m, 2H), 3.30-3.23 (m, 2H), 2.94 (dd, J=6.1, 11.9 Hz, 2H), 1.22 (d, J=6.4 Hz, 6H); LCMS: 360.1 [M+H]⁺.

The Compounds below were synthesized from the appropriate Intermediate and the appropriate amine following the procedures described for Compound 20.

| Cmpd | Structure | Name | [M + H]⁺ |
|---|---|---|---|
| 20.1 | 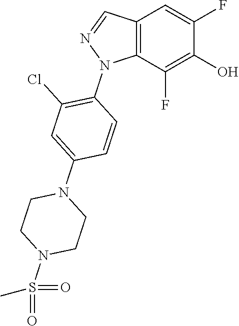 [SMILES: CS(=O)(=O)N1CCN(c2ccc(-n3ncc4cc(F)c(O)c(F)c43)c(Cl)c2)CC1] | 1-(2-Chloro-4-(4- (methylsulfonyl)piperazin-1- yl)phenyl)-5,7-difluoro-1H- indazol-6-ol | 442.9 |
| 20.2 | 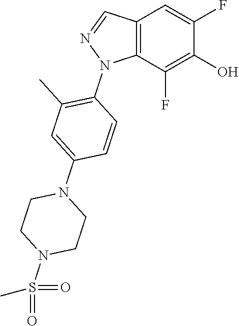 [SMILES: Cc1cc(N2CCN(S(C)(=O)=O)CC2)ccc1-n1ncc2cc(F)c(O)c(F)c21] | 5,7-Difluoro-1-(2-methyl-4-(4- (methylsulfonyl)piperazin-1- yl)phenyl)-1H-indazol-6-ol | 423.0 |
| 20.3 | 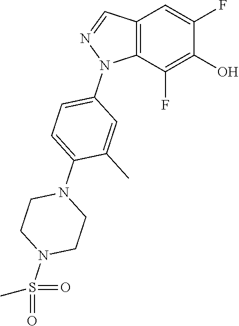 [SMILES: Cc1cc(-n2ncc3cc(F)c(O)c(F)c32)ccc1N1CCN(S(C)(=O)=O)CC1] | 5,7-Difluoro-1-(3-methyl-4-(4- (methylsulfonyl)piperazin-1- yl)phenyl)-1H-indazol-6-ol | 422.9 |
| 20.4 | 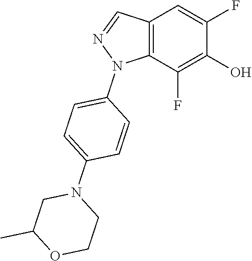 [SMILES: CC1CN(c2ccc(-n3ncc4cc(F)c(O)c(F)c43)cc2)CCO1] | 5,7-Difluoro-1-(4-(2- methylmorpholino)phenyl)-1H- indazol-6-ol | 346.0 |
| 20.5 | 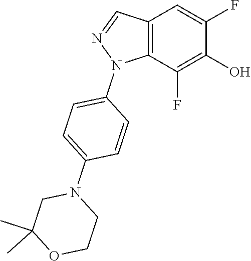 [SMILES: CC1(C)CN(c2ccc(-n3ncc4cc(F)c(O)c(F)c43)cc2)CCO1] | 1-(4-(2,2- Dimethylmorpholino)phenyl)-5,7- difluoro-1H-indazol-6-ol | 360.1 |
| 20.6 | 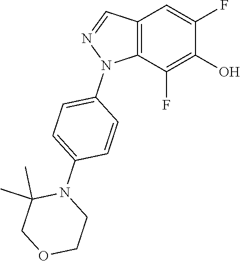 [SMILES: CC1(C)COCCN1c1ccc(-n2ncc3cc(F)c(O)c(F)c32)cc1] | 1-(4-(3,3- Dimethylmorpholino)phenyl)-5,7- difluoro-1H-indazol-6-ol | 360.0 |
| 20.7 | 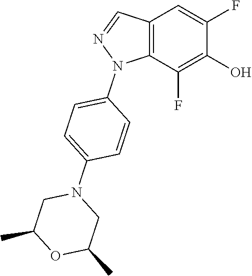 | 1-(4-(cis-2,6- Dimethylmorpholino)phenyl)-5,7- difluoro-1H-indazol-6-ol | 360.1 |
| 20.8 | 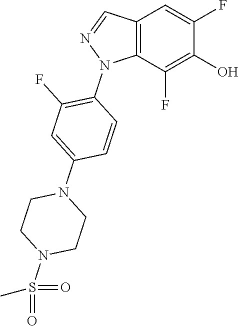 [SMILES: CS(=O)(=O)N1CCN(c2ccc(-n3ncc4cc(F)c(O)c(F)c43)c(F)c2)CC1] | 5,7-Difluoro-1-(2-fluoro-4-(4- (methylsulfonyl)piperazin-1- yl)phenyl)-1H-indazol-6-ol | 427.0 |
| 20.9¹ | 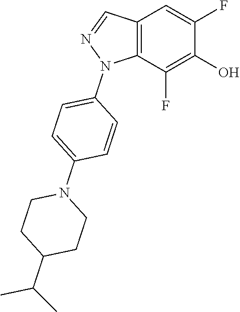 [SMILES: CC(C)C1CCN(c2ccc(-n3ncc4cc(F)c(O)c(F)c43)cc2)CC1] | 5,7-Difluoro-1-(4-(4- isopropylpiperidin-1-yl)phenyl)- 1H-indazol-6-ol | 372.3 |
| 20.10¹ | 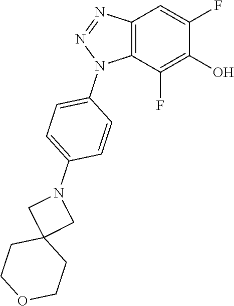 [SMILES: Oc1c(F)cc2nnn(-c3ccc(N4CC5(CCOCC5)C4)cc3)c2c1F] | 1-(4-(7-Oxa-2-azaspiro[3.5]nonan- 2-yl)phenyl)-5,7-difluoro-1H- benzo[d][1,2,3]triazol-6-ol | 373.0 |
| 20.11¹ | 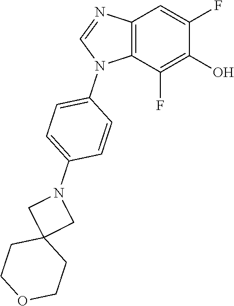 [SMILES: Oc1c(F)cc2ncn(-c3ccc(N4CC5(CCOCC5)C4)cc3)c2c1F] | 1-(4-(7-Oxa-2-azaspiro[3.5]nonan- 2-yl)phenyl)-5,7-difluoro-1H- benzo[d]imidazol-6-ol | 372.1 |
| 20.12¹ | 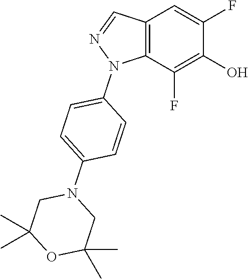 [SMILES: CC1(C)CN(c2ccc(-n3ncc4cc(F)c(O)c(F)c43)cc2)CC(C)(C)O1] | 5,7-Difluoro-1-(4-(2,2,6,6- tetramethylmorpholino)phenyl)- 1H-indazol-6-ol | 388.1 |
| 20.13¹ | 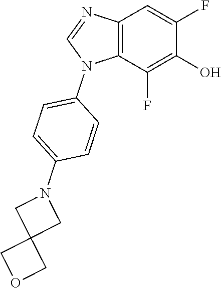 [SMILES: Oc1c(F)cc2ncn(-c3ccc(N4CC5(COC5)C4)cc3)c2c1F] | 1-(4-(2-Oxa-6- azaspiro[3.3]heptan-6-yl)phenyl)- 5,7-difluoro-1H-indazol-6-ol | 344.0 |
| 20.14¹ | 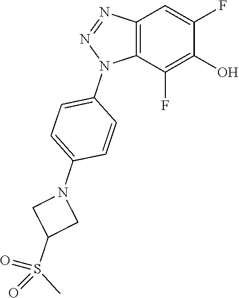 [SMILES: CS(=O)(=O)C1CN(c2ccc(-n3nnc4cc(F)c(O)c(F)c43)cc2)C1] | 5,7-Difluoro-1-(4-(3- (methylsulfonyl)azetidin-1- yl)phenyl)-1H- benzo[d][1,2,3]triazol-6-ol | 380.9 |
| 20.15¹ | 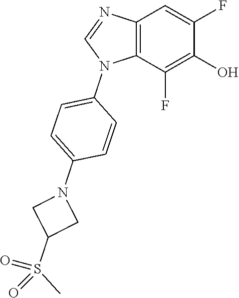 [SMILES: CS(=O)(=O)C1CN(c2ccc(-n3cnc4cc(F)c(O)c(F)c43)cc2)C1] | 5,7-Difluoro-1-(4-(3- (methylsulfonyl)azetidin-1- yl)phenyl)-1H-benzo[d]imidazol- 6-ol | 380.0 |
| 20.16¹ | 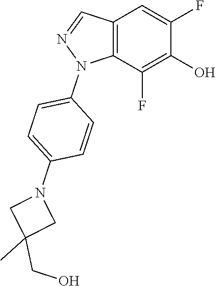 [SMILES: CC1(CO)CN(c2ccc(-n3ncc4cc(F)c(O)c(F)c43)cc2)C1] | 5,7-Difluoro-1-(4-(3- (hydroxymethyl)-3- methylazetidin-1-yl)phenyl)-1H- indazol-6-ol | 346.0 |
| 20.17¹ | 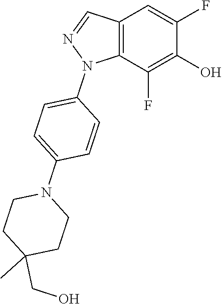 [SMILES: CC1(CO)CCN(c2ccc(-n3ncc4cc(F)c(O)c(F)c43)cc2)CC1] | 5,7-Difluoro-1-(4-(4- (hydroxymethyl)-4- methylpiperidin-1-yl)phenyl)-1H- indazol-6-ol | 374.1 |
| 20.18¹ | 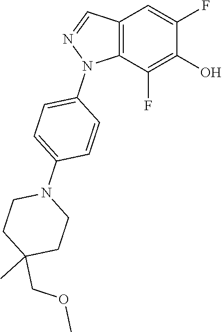 [SMILES: COCC1(C)CCN(c2ccc(-n3ncc4cc(F)c(O)c(F)c43)cc2)CC1] | 5,7-Difluoro-1-(4-(4- (methoxymethyl)-4- methylpiperidin-1-yl)phenyl)-1H- indazol-6-ol | 388.3 |
| 20.19¹ | 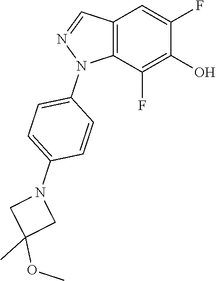 [SMILES: COC1(C)CN(c2ccc(-n3ncc4cc(F)c(O)c(F)c43)cc2)C1] | 5,7-Difluoro-1-(4-(3-methoxy-3- methylazetidin-1-yl)phenyl)-1H- indazol-6-ol | 346.0 |
| 20.20¹ | 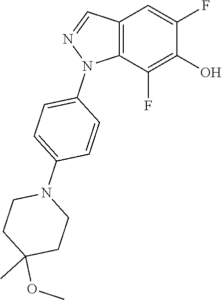 [SMILES: COC1(C)CCN(c2ccc(-n3ncc4cc(F)c(O)c(F)c43)cc2)CC1] | 5,7-Difluoro-1-(4-(4-methoxy-4- methylpiperidin-1-yl)phenyl)-1H- indazol-6-ol | 374.3 |
| 20.21¹ | 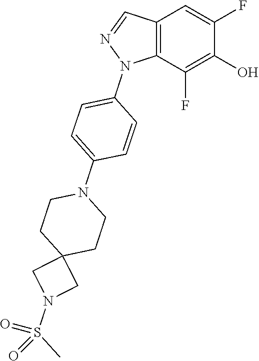 [SMILES: CS(=O)(=O)N1CC2(CCN(c3ccc(-n4ncc5cc(F)c(O)c(F)c54)cc3)CC2)C1] | 5,7-Difluoro-1-(4-(2- (methylsulfonyl)-2,7- diazaspiro[3.5]nonan-7- yl)phenyl)-1H-indazol-6-ol | 449.1 |
| 20.22¹ | 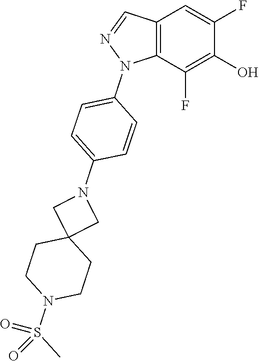 [SMILES: CS(=O)(=O)N1CCC2(CC1)CN(c1ccc(-n3ncc4cc(F)c(O)c(F)c43)cc1)C2] | 5,7-Difluoro-1-(4-(7- (methylsulfonyl)-2,7- diazaspiro[3.5]nonan-2- yl)phenyl)-1H-indazol-6-ol | 449.0 |
| 20.23¹ | 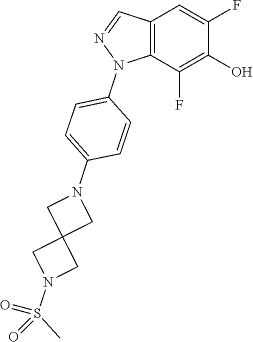 [SMILES: CS(=O)(=O)N1CC2(CN(c3ccc(-n4ncc5cc(F)c(O)c(F)c54)cc3)C2)C1] | 5,7-Difluoro-1-(4-(6- (methylsulfonyl)-2,6- diazaspiro[3.3]heptan-2- yl)phenyl)-1H-indazol-6-ol | 421.0 |

Alternate conditions: Step 1: Amine was also an HCl salt; 80 min-25 h. Step 2: 150-170° C., 20 min-4 h.
¹Step 1 only when Intermediate with unprotected phenol was used.

### Compound 21

### 1-(4-(4,4-Dimethylpiperidin-1-yl)phenyl)-5,7-difluoro-1H-benzo[d][1,2,3]triazol-6-ol

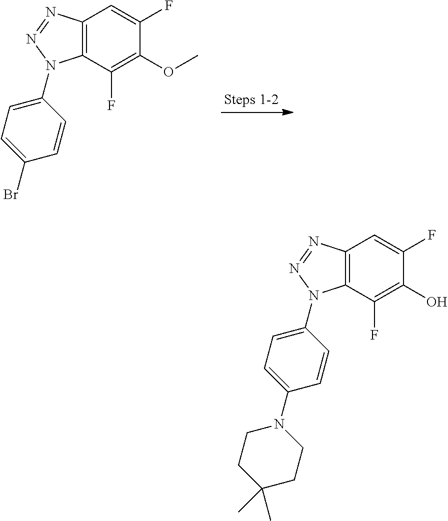 [SMILES: COc1c(F)cc2nnn(-c3ccc(Br)cc3)c2c1F ; CC1(C)CCN(c2ccc(-n3nnc4cc(F)c(O)c(F)c43)cc2)CC1]

### Step 1: 1-(4-(4,4-Dimethylpiperidin-1-yl)phenyl)-5,7-difluoro-6-methoxy-1H-benzo[d][1,2,3]triazole

A mixture of Intermediate 15 (60 mg, 0.18 mmol), 4,4-dimethylpiperidine hydrochloride (39 mg, 0.26 mmol), tris(dibenzylideneacetone)dipalladium (32 mg, 0.035 mmol), RuPhos (12 mg, 0.026 mmol), sodium tert-butoxide (30 mg, 0.31 mmol), and toluene (1 mL) was degassed with 2 vacuum/N₂ cycles, stirred at 100° C. for 67 h, allowed to cool to rt, diluted with EtOAc (10 mL) and water (5 mL), and then filtered through Celite. The filter cake was washed with EtOAc (5 mL). The organic layer was washed with brine (5 mL), dried (Na₂SO₄), filtered, concentrated, and then purified by silica gel chromatography (0-25% EtOAc in hexanes) to give 1-(4-(4,4-dimethylpiperidin-1-yl)phenyl)-5,7-difluoro-6-methoxy-1H-benzo[d][1,2,3]triazole (41 mg, 62%) as a yellow solid. ¹HNMR (400 MHz, DMSO-d₆): δ 8.02 (dd, J=1.4, 9.9 Hz, 1H), 7.55 (dd, J=2.0, 9.0 Hz, 2H), 7.13 (d, J=8.8 Hz, 2H), 4.02 (s, 3H), 3.34-3.28 (m, 4H), 1.50-1.44 (m, 4H), 0.99 (s, 6H); LCMS: 373.2 [M+H]⁺.

### Step 2: 1-(4-(4,4-Dimethylpiperidin-1-yl)phenyl)-5,7-difluoro-1H-benzo[d][1,2,3]triazol-6-ol

Boron tribromide (1 M in CH₂Cl₂, 0.45 mL, 0.45 mmol) was added slowly to a mixture of (1-(4-(4,4-dimethylpiperidin-1-yl)phenyl)-5,7-difluoro-6-methoxy-1H-benzo[d][1,2,3]triazole (37 mg, 0.10 mmol) in CH₂Cl₂ (1 mL) at 0° C. under N₂. The mixture was stirred at 0° C. for 15 min, stirred at 40° C. for 1 h, cooled in an ice/water bath, quenched slowly with CH₃OH (2 mL), concentrated, and then diluted with EtOAc (15 mL). The organic layer was washed with 1.0 M NaOH (10×15 mL because of B-coordination), washed with brine (10 mL), dried (Na₂SO₄), filtered, concentrated, and then purified by silica gel chromatography (0-30% EtOAc in CH₂Cl₂) to give 1-(4-(4,4-dimethylpiperidin-1-yl)phenyl)-5,7-difluoro-1H-benzo[d][1,2,3]triazol-6-ol (18 mg, 51%) as a pale yellow solid. ¹HNMR (400 MHz, DMSO-d₆): δ 10.94 (s, 1H), 7.90 (dd, J=1.3, 9.8 Hz, 1H), 7.5 (br d, J=7.5 Hz, 2H), 7.12 (d, J=8.9 Hz, 2H), 3.32-3.26 (m, 4H), 1.50-1.43 (m, 4H), 0.99 (s, 6H); LCMS: 359.1 [M+H]⁺.

The Compounds below were synthesized from the appropriate Intermediate and the appropriate amine following the procedures described for Compound 21.

| Cmpd | Structure | Name | [M + H]⁺ |
|---|---|---|---|
| 21.1 | 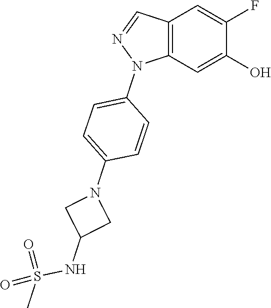 [SMILES: CS(=O)(=O)NC1CN(c2ccc(-n3ncc4cc(F)c(O)cc43)cc2)C1] | N-(1-(4-(5-Fluoro-6-hydroxy-1H- indazol-1-yl)phenyl)azetidin-3- yl)methanesulfonamide | 376.9 |
| 21.2 | 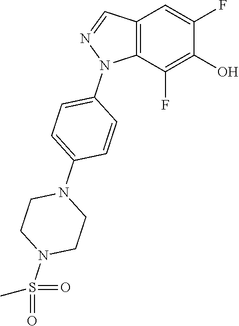 [SMILES: CS(=O)(=O)N1CCN(c2ccc(-n3ncc4cc(F)c(O)c(F)c43)cc2)CC1] | 5-Fluoro-1-(4-(4- (methylsulfonyl)piperazin-1- yl)phenyl)-1H-indazol-6-ol | 390.9 |
| 21.3 | 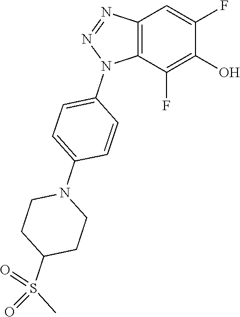 [SMILES: CS(=O)(=O)C1CCN(c2ccc(-n3nnc4cc(F)c(O)c(F)c43)cc2)CC1] | 5,7-Difluoro-1-(4-(4- (methylsulfonyl)piperazin-1- yl)phenyl)-1H- benzo[d][1,2,3]triazol-6-ol | 410.0 |
| 21.4 | 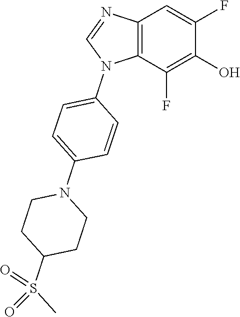 [SMILES: CS(=O)(=O)C1CCN(c2ccc(-n3cnc4cc(F)c(O)c(F)c43)cc2)CC1] | 5,7-Difluoro-1-(4-(4- (methylsulfonyl)piperazin-1- yl)phenyl)-1H-benzo[d]imidazol- 6-ol | 409.2 |
| 21.5 | 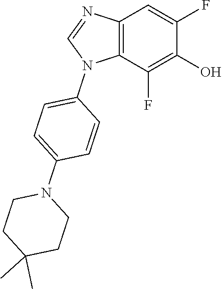 [SMILES: CC1(C)CCN(c2ccc(-n3cnc4cc(F)c(O)c(F)c43)cc2)CC1] | 1-(4-(4,4-Dimethylpiperidin-1- yl)phenyl)-5,7-difluoro-1H- benzo[d]imidazol-6-ol | 358.3 |
| 21.6 | 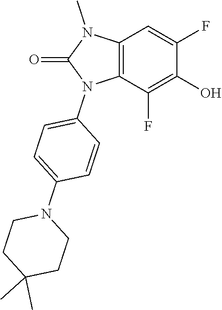 [SMILES: Cn1c(=O)n(-c2ccc(N3CCC(C)(C)CC3)cc2)c2c(F)c(O)c(F)cc21] | 3-(4-(4,4-Dimethylpiperidin-1- yl)phenyl)-4,6-difluoro-5- hydroxy-1-methyl-1H- benzo[d]imidazol-2(3H)-one | 388.2 |
| 21.7 | 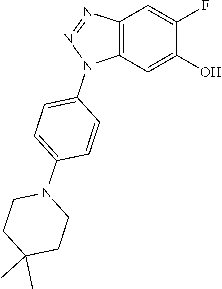 [SMILES: CC1(C)CCN(c2ccc(-n3nnc4cc(F)c(O)cc43)cc2)CC1] | 1-(4-(4,4-Dimethylpiperidin-1- yl)phenyl)-5-fluoro-1H- benzo[d][1,2,3]triazol-6-ol | 341.3 |
| 21.8 | 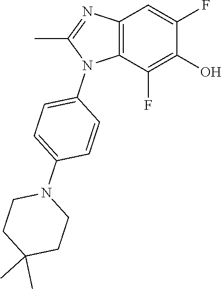 [SMILES: Cc1nc2cc(F)c(O)c(F)c2n1-c1ccc(N2CCC(C)(C)CC2)cc1] | 1-(4-(4,4-Dimethylpiperidin-1- yl)phenyl)-5,7-difluoro-2-methyl- 1H-benzo[d]imidazol-6-ol | 372.2 |
| 21.9 | 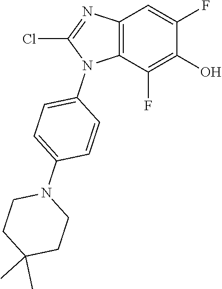 [SMILES: CC1(C)CCN(c2ccc(-n3c(Cl)nc4cc(F)c(O)c(F)c43)cc2)CC1] | 2-Chloro-1-(4-(4,4- dimethylpiperidin-1-yl)phenyl)- 5,7-difluoro-1H-benzo[d]imidazol- 6-ol | 392.1 |
| 21.10 | 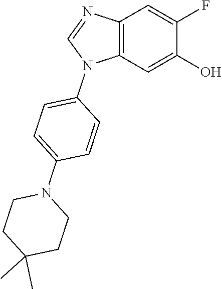 [SMILES: CC1(C)CCN(c2ccc(-n3cnc4cc(F)c(O)cc43)cc2)CC1] | 1-(4-(4,4-Dimethylpiperidin-1- yl)phenyl)-5-fluoro-1H- benzo[d]imidazol-6-ol | 340.1 |
| 21.11 | 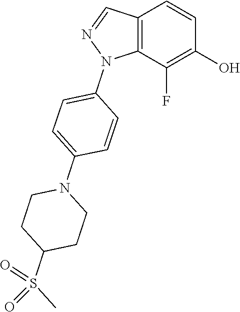 [SMILES: CS(=O)(=O)C1CCN(c2ccc(-n3ncc4ccc(O)c(F)c43)cc2)CC1] | 7-Fluoro-1-(4-(4- (methylsulfonyl)piperazin-1- yl)phenyl)-1H-indazol-6-ol | 391.1 |
| 21.12 | 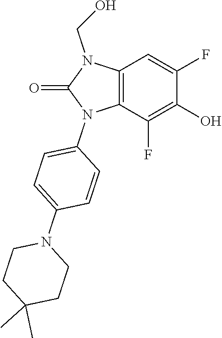 [SMILES: CC1(C)CCN(c2ccc(-n3c(=O)n(CO)c4cc(F)c(O)c(F)c43)cc2)CC1] | 3-(4-(4,4-Dimethylpiperidin-1- yl)phenyl)-4,6-difluoro-5- hydroxy-1-(hydroxymethyl)-1H- benzo[d]imidazol-2(3H)-one | 404.1 |
| 21.13 | 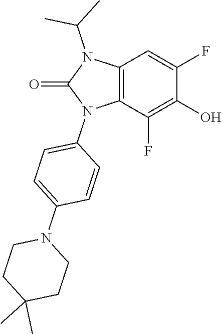 [SMILES: CC(C)n1c(=O)n(-c2ccc(N3CCC(C)(C)CC3)cc2)c2c(F)c(O)c(F)cc21] | 3-(4-(4,4-Dimethylpiperidin-1- yl)phenyl)-4,6-difluoro-5- hydroxy-1-isopropyl-1H- benzo[d]imidazol-2(3H)-one | 416.4 |

Alternate conditions: Step 1: Amine was also an HCl salt; 0.5-90 h. Step 2: 0° C.-rt, 15 min-3 h; also 40° C., 1-5 h.

### Compound 22

### 1-(4-(5,7-Difluoro-6-hydroxy-1H-indazol-1-yl)phenyl)piperidine-4-carbonitrile

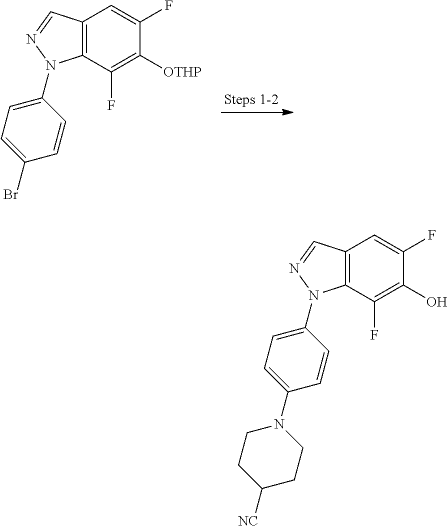

### Step 1: 1-(4-(5,7-Difluoro-6-((tetrahydro-2H-pyran-2-yl)oxy)-1H-indazol-1-yl)phenyl)piperidine-4-carbonitrile

A mixture of Intermediate 24 (57 mg, 0.13 mmol) in dioxane (1.2 mL), piperidine-4-carbonitrile (20 mg, 0.18 mmol), and cesium carbonate (88 mg, 0.27 mmol) were combined in a 4 mL vial at rt. The mixture was degassed with 3 vacuum/N₂ cycles. Tris(dibenzylideneacetone)-dipalladium(0) (7 mg, 0.01 mmol) and XPhos (7.6 mg, 0.02 mmol) were added. The reaction was stirred at 90° C. for 1.5 h, allowed to cool to rt, and then diluted with 20 mL ethyl acetate and 20 mL water. The layers were separated. The ethyl acetate layer was washed with 20 mL brine, dried (Na₂SO₄), filtered, concentrated, and then purified by silica gel chromatography (15-35% ethyl acetate in hexanes) to give 1-(4-(5,7-difluoro-6-((tetrahydro-2H-pyran-2-yl)oxy)-1H-indazol-1-yl)phenyl)piperidine-4-carbonitrile (48 mg, 82%) as an off-white solid. ¹H NMR (400 MHz, DMSO-d₆): δ 8.32 (d, J=2.2 Hz, 1H), 7.62 (d, J=9.2 Hz, 1H), 7.43 (dd, J=2.6, 9.0 Hz, 2H), 7.08 (d, J=9.0 Hz, 2H), 5.42 (s, 1H), 3.96 (td, J=6.9, 11.2 Hz, 1H), 3.59-3.52 (m, 1H), 3.50-3.42 (m, 2H), 3.21-3.11 (m, 2H), 3.11-3.05 (m, 1H), 2.06-1.96 (m, 2H), 1.94-1.79 (m, 5H), 1.68-1.53 (m, 3H); LCMS: 439.1 [M+H]⁺.

### Step 2: 1-(4-(5,7-Difluoro-6-hydroxy-1H-indazol-1-yl)phenyl)piperidine-4-carbonitrile

Aqueous hydrochloric acid (1 N, 0.15 mL, 0.15 mmol) was added to a mixture of 1-(4-(5,7-difluoro-6-((tetrahydro-2H-pyran-2-yl)oxy)-1H-indazol-1-yl)phenyl)piperidine-4-carbonitrile (44.3 mg, 0.10 mmol), methanol (1 mL), and tetrahydrofuran (1 mL) at rt. The reaction was stirred for 1 h and then diluted with 20 mL water and 20 mL ethyl acetate. The layers were separated. The organic layer was washed with 20 mL saturated NaHCO₃, washed with 20 mL brine, dried (Na₂SO₄), filtered, concentrated, and then dried on high vacuum to give 1-(4-(5,7-difluoro-6-hydroxy-1H-indazol-1-yl)phenyl)piperidine-4-carbonitrile (32 mg, 90%) as a tan solid. ¹H NMR (400 MHz, DMSO-d₆): δ 10.45 (s, 1H), 8.21 (d, J=2.2 Hz, 1H), 7.52-7.46 (m, 1H), 7.40 (dd, J=2.7, 8.9 Hz, 2H), 7.07 (d, J=8.9 Hz, 2H), 3.50-3.40 (m, 2H), 3.19-3.04 (m, 3H), 2.06-1.96 (m, 2H), 1.90-1.79 (m, 2H); LCMS: 355.0 [M+H]⁺.

The Compounds below were synthesized from Intermediate 24 and the appropriate amine following the procedures described for Compound 22.

| Cmpd | Structure | Name | [M + H]⁺ |
|---|---|---|---|
| 22.1 | 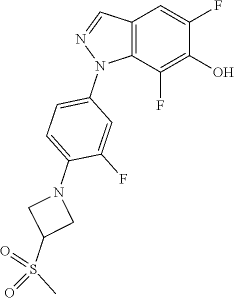 [SMILES: CS(=O)(=O)C1CN(c2ccc(-n3ncc4cc(F)c(O)c(F)c43)cc2F)C1] | 5,7-Difluoro-1-(4-(3- (methylsulfonyl)azetidin-1- yl)phenyl)-1H-indazol-6-ol | 380.0 |
| 22.2 | 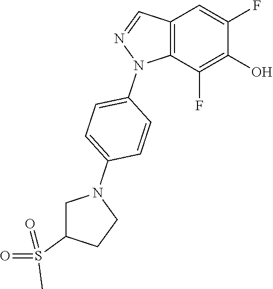 [SMILES: CS(=O)(=O)C1CCN(c2ccc(-n3ncc4cc(F)c(O)c(F)c43)cc2)C1] | 5,7-Difluoro-1-(4-(3- (methylsulfonyl)pyrrolidin-1- yl)phenyl)-1H-indazol-6-ol | 393.9 |
| 22.3 | 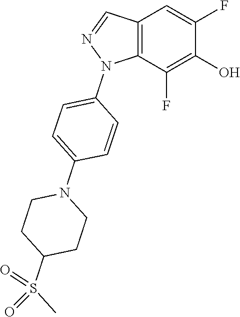 [SMILES: CS(=O)(=O)C1CCN(c2ccc(-n3ncc4cc(F)c(O)c(F)c43)cc2)CC1] | 5,7-Difluoro-1-(4-(4- (methylsulfonyl)piperidin-1- yl)phenyl)-1H-indazol-6-ol | 408.0 |
| 22.4 | 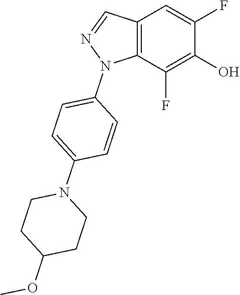 [SMILES: COC1CCN(c2ccc(-n3ncc4cc(F)c(O)c(F)c43)cc2)CC1] | 5,7-Difluoro-1-(4-(4- methoxypiperidin-1-yl)phenyl)- 1H-indazol-6-ol | 360.0 |
| 22.5 | 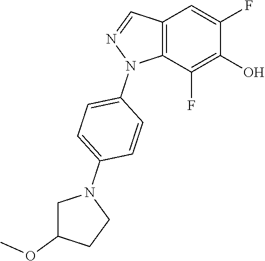 [SMILES: COC1CCN(c2ccc(-n3ncc4cc(F)c(O)c(F)c43)cc2)C1] | 5,7-Difluoro-1-(4-(3- methoxypyrrolidin-1-yl)phenyl)- 1H-indazol-6-ol | 346.0 |

Alternate conditions: Step 1: Amine was also an HCl salt; 1.5-21 h.

### Compound 23

### 1-(4-(4,4-Dimethylpiperidin-1-yl)phenyl)-5,7-difluoro-LH-indazol-6-ol

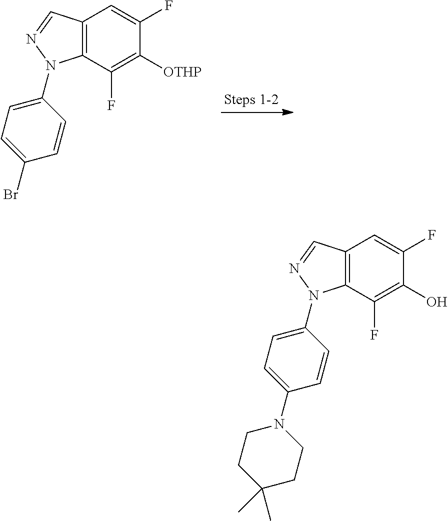

### Step 1: 1-(4-(4,4-Dimethylpiperidin-1-yl)phenyl)-5,7-difluoro-6-((tetrahydro-2H-pyran-2-yl)oxy)-1H-indazole

Sodium tert-butoxide (64 mg, 0.67 mmol) was weighed into a 4 mL vial, placed under high vacuum, and then heated with a heat gun for a couple min (until solids stopped popping). The vial was allowed to cool to rt under vacuum. 4,4-Dimethylpiperidine hydrochloride (48 mg, 0.32 mmol) and then Intermediate 24 (64 mg, 0.16 mmol) in toluene (1 mL) were added to the vial. Additional toluene (0.5 mL) was added to rinse the vial. The reaction mixture was degassed with 3 vacuum/N₂ cycles.

Tris(dibenzylideneacetone)dipalladium(0) (7.2 mg, 0.01 mmol) and RuPhos (7.4 mg, 0.02 mmol) were added. The reaction was stirred at 100° C. for 1 h, allowed to cool to rt, and then diluted with 20 mL ethyl acetate. The organic layer was washed with 20 mL water, washed with 20 mL brine, dried (Na₂SO₄), filtered, concentrated, and then purified by silica gel chromatography (0-20% ethyl acetate in hexanes) to give 1-(4-(4,4-dimethylpiperidin-1-yl)phenyl)-5,7-difluoro-1H-indazol-6-ol (58 mg, 84%) as a pale yellow solid. ¹H NMR (400 MHz, DMSO-d₆): δ 8.31 (d, J=2.2 Hz, 1H), 7.65-7.58 (m, 1H), 7.40 (dd, J=2.4, 8.9 Hz, 2H), 7.05 (br d, J=8.7 Hz, 2H), 5.42 (s, 1H), 3.96 (td, J=7.0, 11.1 Hz, 1H), 3.55 (td, J=3.6, 10.6 Hz, 1H), 3.29-3.20 (m, 4H), 1.95-1.79 (m, 3H), 1.68-1.53 (m, 3H), 1.49-1.42 (m, 4H), 0.98 (s, 6H); LCMS: 442.1 [M+H]⁺.

### Step 2: 1-(4-(4,4-Dimethylpiperidin-1-yl)phenyl)-5,7-difluoro-1H-indazol-6-ol

Aqueous hydrochloric acid (1 N, 0.20 mL, 0.20 mmol) was added to a mixture of 6-(2H-3,4,5,6-tetrahydropyran-2-yloxy)-1-[4-(4,4-dimethylpiperidyl)phenyl]-5,7-difluoro-1H-indazole (55 mg, 0.12 mmol), methanol (1 mL), and toluene (1 mL) at rt. The reaction was stirred for 40 min and then diluted with 20 mL ethyl acetate and 20 mL water. The layers were separated. The organic layer was washed with 20 mL brine, dried (Na₂SO₄), filtered, concentrated, and then purified by silica gel chromatography (5-30% ethyl acetate in hexanes) to give 1-(4-(4,4-dimethylpiperidin-1-yl)phenyl)-5,7-difluoro-1H-indazol-6-ol (40 mg, 81%) as a white solid. ¹HNMR (400 MHz, DMSO-d₆): δ 10.44 (s, 1H), 8.20 (d, J=2.2 Hz, 1H), 7.49 (dd, J=1.0, 9.8 Hz, 1H), 7.37 (dd, J=2.6, 9.0 Hz, 2H), 7.04 (br d, J=8.8 Hz, 2H), 3.27-3.21 (m, 4H), 1.49-1.43 (m, 4H), 0.98 (s, 6H); LCMS: 358.3 [M+H]⁺.

The Compounds below were synthesized from Intermediate 24 and the appropriate amine following the procedures described for Compound 23.

| Cmpd | Structure | Name | [M + H]⁺ |
|---|---|---|---|
| 23.1 | 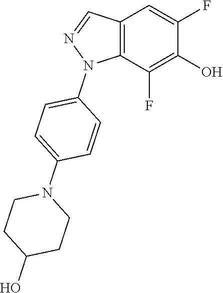 [SMILES: Oc1c(F)cc2cnn(-c3ccc(N4CCC(O)CC4)cc3)c2c1F] | 5,7-Difluoro-1-(4-(4- hydroxypiperidin-1-yl)phenyl)- 1H-indazol-6-ol | 346.0 |
| 23.2 | 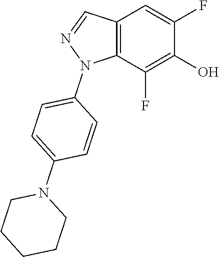 [SMILES: Oc1c(F)cc2cnn(-c3ccc(N4CCCCC4)cc3)c2c1F] | 5,7-Difluoro-1-(4-(piperidin-1- yl)phenyl)-1H-indazol-6-ol | 330.2 |
| 23.3 | 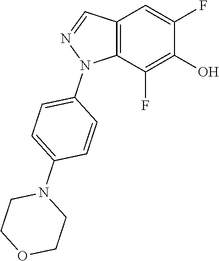 [SMILES: Oc1c(F)cc2cnn(-c3ccc(N4CCOCC4)cc3)c2c1F] | 5,7-Difluoro-1-(4- morpholinophenyl)-1H-indazol- 6-ol | 332.0 |
| 23.4 | 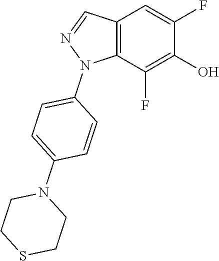 [SMILES: Oc1c(F)cc2cnn(-c3ccc(N4CCSCC4)cc3)c2c1F] | 5,7-Difluoro-1-(4- thiomorpholinophenyl)-1H- indazol-6-ol | 348.0 |
| 23.5 | 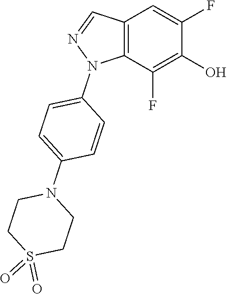 [SMILES: O=S1(=O)CCN(c2ccc(-n3ncc4cc(F)c(O)c(F)c43)cc2)CC1] | 4-(4-(5,7-Difluoro-6-hydroxy-1H- indazol-1- yl)phenyl)thiomorpholine 1,1- dioxide | 379.9 |
| 23.6 | 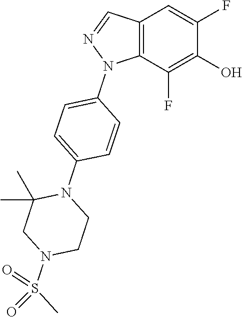 [SMILES: CC1(C)CN(S(C)(=O)=O)CCN1c1ccc(-n2ncc3cc(F)c(O)c(F)c32)cc1] | 1-(4-(2,2-Dimethyl-4- (methylsulfonyl)piperazin-1- yl)phenyl)-5,7-difluoro-1H- indazol-6-ol | 437.0 |
| 23.7 | 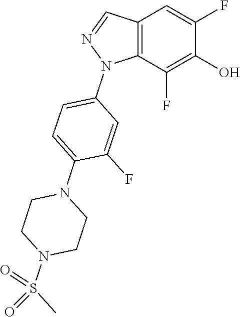 [SMILES: CS(=O)(=O)N1CCN(c2ccc(-n3ncc4cc(F)c(O)c(F)c43)cc2F)CC1] | 5,7-Difluoro-1-(3-fluoro-4-(4- (methylsulfonyl)piperazin-1- yl)phenyl)-1H-indazol-6-ol | 427.0 |
| 23.8 | 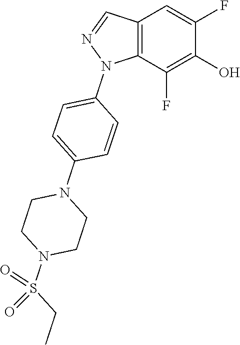 [SMILES: CCS(=O)(=O)N1CCN(c2ccc(-n3ncc4cc(F)c(O)c(F)c43)cc2)CC1] | 1-(4-(4-(Ethylsulfonyl)piperazin- 1-yl)phenyl)-5,7-difluoro-1H- indazol-6-ol | 423.0 |
| 23.9 | 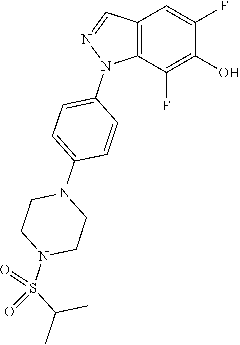 [SMILES: CC(C)S(=O)(=O)N1CCN(c2ccc(-n3ncc4cc(F)c(O)c(F)c43)cc2)CC1] | 5,7-Difluoro-1-(4-(4- (isopropylsulfonyl)piperazin-1- yl)phenyl)-1H-indazol-6-ol | 437.1 |
| 23.10 | 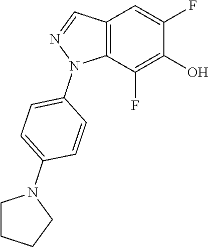 [SMILES: Oc1c(F)cc2cnn(-c3ccc(N4CCCC4)cc3)c2c1F] | 5,7-Difluoro-1-(4-(pyrrolidin-1- yl)phenyl)-1H-indazol-6-ol | 316.1 |
| 23.11 | 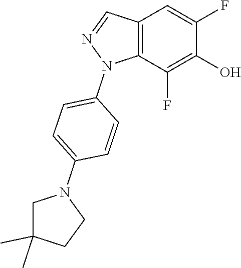 [SMILES: CC1(C)CCN(c2ccc(-n3ncc4cc(F)c(O)c(F)c43)cc2)C1] | 1-(4-(3,3-Dimethylpyrrolidin-1- yl)phenyl)-5,7-difluoro-1H- indazol-6-ol | 344.1 |
| 23.12 | 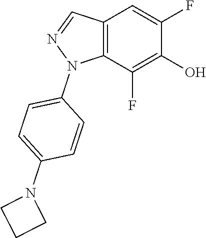 [SMILES: Oc1c(F)cc2cnn(-c3ccc(N4CCC4)cc3)c2c1F] | 1-(4-(Azetidin-1-yl)phenyl)-5,7- difluoro-1H-indazol-6-ol | 301.9 |
| 23.13 | 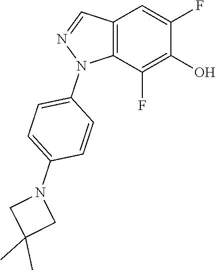 [SMILES: CC1(C)CN(c2ccc(-n3ncc4cc(F)c(O)c(F)c43)cc2)C1] | 1-(4-(3,3-Dimethylazetidin-1- yl)phenyl)-5,7-difluoro-1H- indazol-6-ol | 330.0 |
| 23.14 | 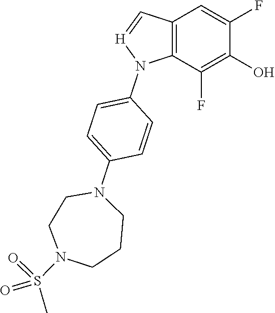 | 5,7-Difluoro-1-(4-(4- (methylsulfonyl)-1,4-diazepan-1- yl)phenyl)-1H-indazol-6-ol | 423.0 |
| 23.15 | 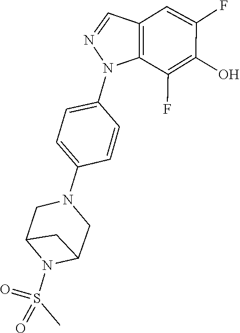 [SMILES: CS(=O)(=O)N1C2CC1CN(c1ccc(-n3ncc4cc(F)c(O)c(F)c43)cc1)C2] | 5,7-Difluoro-1-(4-(6- (methylsulfonyl)-3,6- diazabicyclo[3.1.1]heptan-3- yl)phenyl)-1H-indazol-6-ol | 421.0 |
| 23.16 | 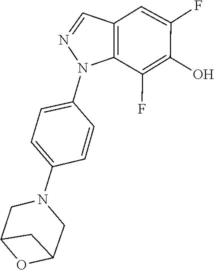 [SMILES: Oc1c(F)cc2cnn(-c3ccc(N4CC5CC(C4)O5)cc3)c2c1F] | 1-(4-(6-Oxa-3- azabicyclo[3.1.1]heptan-3- yl)phenyl)-5,7-difluoro-1H- indazol-6-ol | 344.0 |
| 23.17 | 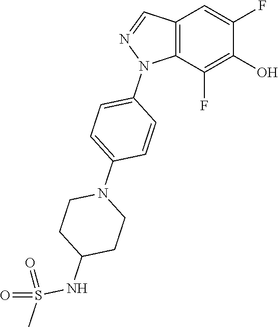 [SMILES: CS(=O)(=O)NC1CCN(c2ccc(-n3ncc4cc(F)c(O)c(F)c43)cc2)CC1] | N-(1-(4-(5,7-Difluoro-6-hydroxy- 1H-indazol-1-yl)phenyl)piperidin- 4-yl)methanesulfonamide | 423.1 |
| 23.18 | 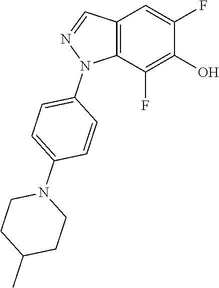 [SMILES: CC1CCN(c2ccc(-n3ncc4cc(F)c(O)c(F)c43)cc2)CC1] | 5,7-Difluoro-1-(4-(4- methylpiperidin-1-yl)phenyl)-1H- indazol-6-ol | 344.2 |
| 23.19 | 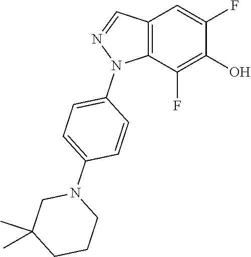 [SMILES: CC1(C)CCCN(c2ccc(-n3ncc4cc(F)c(O)c(F)c43)cc2)C1] | 1-(4-(3,3-Dimethylpiperidin-1- yl)phenyl)-5,7-difluoro-1H- indazol-6-ol | 358.2 |
| 23.20 | 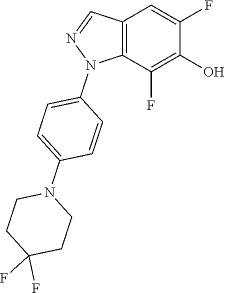 [SMILES: Oc1c(F)cc2cnn(-c3ccc(N4CCC(F)(F)CC4)cc3)c2c1F] | 1-(4-(4,4-Difluoropiperidin-1- yl)phenyl)-5,7-difluoro-1H- indazol-6-ol | 366.1 |
| 23.21 | 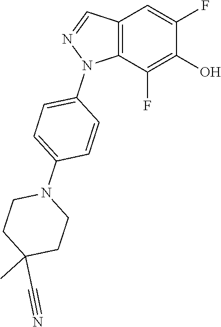 [SMILES: CC1(C#N)CCN(c2ccc(-n3ncc4cc(F)c(O)c(F)c43)cc2)CC1] | 1-(4-(5,7-Difluoro-6-hydroxy-1H- indazol-1-yl)phenyl)-4- methylpiperidine-4-carbonitrile | 369.3 |
| 23.22 | 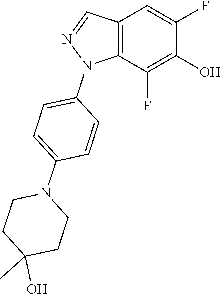 [SMILES: CC1(O)CCN(c2ccc(-n3ncc4cc(F)c(O)c(F)c43)cc2)CC1] | 5,7-Difluoro-1-(4-(4-hydroxy-4- methylpiperidin-1-yl)phenyl)-1H- indazol-6-ol | 360.2 |
| 23.23 | 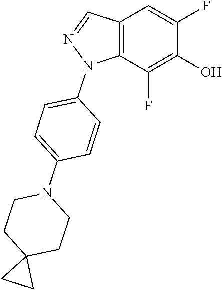 [SMILES: Oc1c(F)cc2cnn(-c3ccc(N4CCC5(CC4)CC5)cc3)c2c1F] | 1-(4-(6-Azaspiro[2.5]octan-6- yl)phenyl)-5,7-difluoro-1H- indazol-6-ol | 356.1 |
| 23.24 | 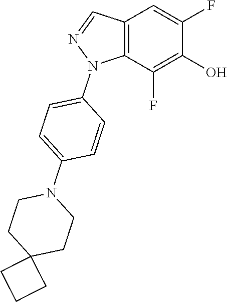 [SMILES: Oc1c(F)cc2cnn(-c3ccc(N4CCC5(CCC5)CC4)cc3)c2c1F] | 1-(4-(7-Azaspiro[3.5]nonan-7- yl)phenyl)-5,7-difluoro-1H- indazol-6-ol | 370.1 |
| 23.25 | 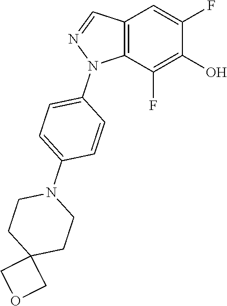 [SMILES: Oc1c(F)cc2cnn(-c3ccc(N4CCC5(CC4)COC5)cc3)c2c1F] | 1-(4-(2-Oxa-7-azaspiro[3.5]nonan- 7-yl)phenyl)-5,7-difluoro-1H- indazol-6-ol | 372.2 |
| 23.26 | 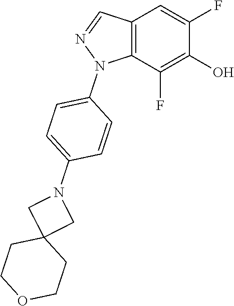 [SMILES: Oc1c(F)cc2cnn(-c3ccc(N4CC5(CCOCC5)C4)cc3)c2c1F] | 1-(4-(7-Oxa-2-azaspiro[3.5]nonan- 2-yl)phenyl)-5,7-difluoro-1H- indazol-6-ol | 372.1 |
| 23.27 | 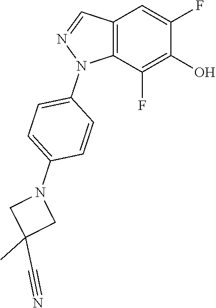 [SMILES: CC1(C#N)CN(c2ccc(-n3ncc4cc(F)c(O)c(F)c43)cc2)C1] | 1-(4-(5,7-Difluoro-6-hydroxy-1H- indazol-1-yl)phenyl)-3- methylazetidine-3-carbonitrile | 341.0 |
| 23.28¹ | 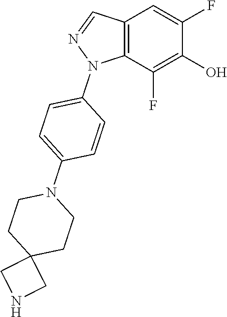 [SMILES: Oc1c(F)cc2cnn(-c3ccc(N4CCC5(CC4)CNC5)cc3)c2c1F] | 1-(4-(2,7-Diazaspiro[3.5]nonan-7- yl)phenyl)-5,7-difluoro-1H- indazol-6-ol | 371.1 |

Alternate conditions: Step 1: Amine was also a free base; 90-100° C.; 40 min-21 h. Step 2: 2M HCl in Et₂O; CH₃OH with THE or DCM; 30 min-2.5 h.
¹Synthesized from tert-butyl 2,7-diazaspiro[3.5]nonane-2-carboxylate followed by BOC/THP deprotection (2 M HCl in Et20, 2:1 Et₂O:CH₃OH, rt, 4 days).

### Compound 24

### 1-(4-(3,3-Diethylazetidin-1-yl)phenyl)-5,7-difluoro-1H-indazol-6-ol

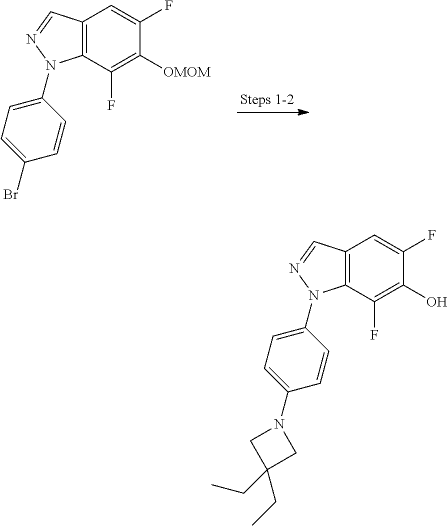

### Step 1: 1-(4-(3,3-Diethylazetidin-1-yl)phenyl)-5,7-difluoro-6-(methoxymethoxy)-1H-indazole

Intermediate 14.8 (107 mg, 0.26 mmol), 3,3-diethylazetidine hydrochloride (82 mg, 0.55 mmol), sodium tert-butoxide* (100 mg, 1.04 mmol), tris(dibenzylideneacetone)dipalladium(0) (13.3 mg, 0.01 mmol), RuPhos (14.4 mg, 0.03 mmol), and then toluene (2 mL) were combined in an 8 mL vial. The reaction was degassed with 3 vacuum/N₂ cycles, stirred at 100° C. for 65 min, allowed to cool to rt, diluted with 2 mL EtOAc and 2 mL water, and then poured into a separatory funnel with 20 mL EtOAc and 20 mL saturated NaHCO₃. The organic layer was washed with 20 mL saturated NaHCO₃, washed with 20 mL brine, dried (Na₂SO₄), filtered, concentrated, and then purified by silica gel chromatography (0-10% EtOAc in hexanes) to give 1-(4-(3,3-diethylazetidin-1-yl)phenyl)-5,7-difluoro-6-(methoxymethoxy)-1H-indazole (100 mg, 95%) as a green solid. ¹H NMR (400 MHz, DMSO-d₆): δ 8.30 (s, 1H), 7.62 (d, J=9.7 Hz, 1H), 7.36 (br d, J=7.0 Hz, 2H), 6.50 (d, J=8.6 Hz, 2H), 5.18 (s, 2H), 3.55 (s, 4H), 3.49 (s, 3H), 1.64 (q, J=7.3 Hz, 4H), 0.86 (t, J=7.4 Hz, 6H); LCMS: 402.2 [M+H]⁺.

* Sodium tert-butoxide was dried under high vacuum by heating with a heat gun for a few minutes and then allowing to cool prior to weighing.

### Step 2: 1-(4-(3,3-Diethylazetidin-1-yl)phenyl)-5,7-difluoro-1H-indazol-6-ol

1-[4-(3,3-Diethylazetidinyl)phenyl]-5,7-difluoro-6-(methoxymethoxy)-1H-indazole (97.1 mg, 0.24 mmol) was dissolved in CH₂Cl₂ (2 mL). Trifluoroacetic acid (0.40 mL, 5.22 mmol) was added at rt. The reaction was stirred for 1 h, diluted with 2 mL EtOAc and 2 mL water, and then poured into separatory funnel with 20 mL EtOAc and 20 mL saturated NaHCO₃. The organic layer was washed with 20 mL brine, dried (Na₂SO₄), filtered, concentrated, and then purified by silica gel chromatography (0-20% EtOAc in hexanes) to give 1-(4-(3,3-diethylazetidin-1-yl)phenyl)-5,7-difluoro-1H-indazol-6-ol (69 mg, 80%) as a white solid. ¹H NMR (400 MHz, DMSO-d₆): δ 10.41 (s, 1H), 8.18 (s, 1H), 7.47 (d, J=9.8 Hz, 1H), 7.33 (br d, J=8.1 Hz, 2H), 6.49 (d, J=8.4 Hz, 2H), 3.54 (s, 4H), 1.64 (q, J=7.3 Hz, 4H), 0.86 (t, J=7.3 Hz, 6H); LCMS: 358.0 [M+H]⁺.

The Compounds below were synthesized from Intermediate 14.8 or Intermediate 13.10 and the appropriate amine following the procedures described for Compound 24.

| Cmpd | Structure | Name | [M + H]⁺ |
|---|---|---|---|
| 24.1 | 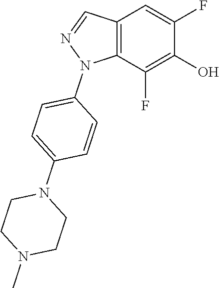 [SMILES: CN1CCN(c2ccc(-n3ncc4cc(F)c(O)c(F)c43)cc2)CC1] | 5,7-Difluoro-1-(4-(4- methylpiperazin-1-yl)phenyl)-1H- indazol-6-ol | 345.0 |
| 24.2 | 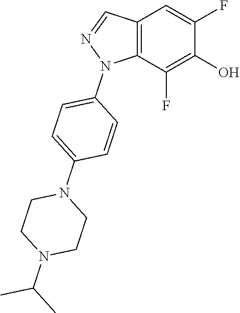 [SMILES: CC(C)N1CCN(c2ccc(-n3ncc4cc(F)c(O)c(F)c43)cc2)CC1] | 5,7-Difluoro-1-(4-(4- isopropylpiperazin-1-yl)phenyl)- 1H-indazol-6-ol | 373.1 |
| 24.3 | 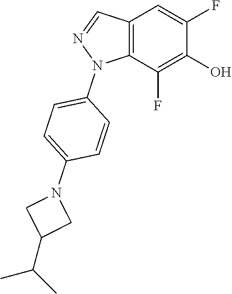 [SMILES: CC(C)C1CN(c2ccc(-n3ncc4cc(F)c(O)c(F)c43)cc2)C1] | 5,7-Difluoro-1-(4-(3- isopropylazetidin-1-yl)phenyl)- 1H-indazol-6-ol | 344.0 |
| 24.4 | 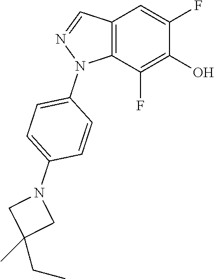 [SMILES: CCC1(C)CN(c2ccc(-n3ncc4cc(F)c(O)c(F)c43)cc2)C1] | 1-(4-(3-Ethyl-3-methylazetidin-1- yl)phenyl)-5,7-difluoro-1H- indazol-6-ol | 344.0 |
| 24.5 | 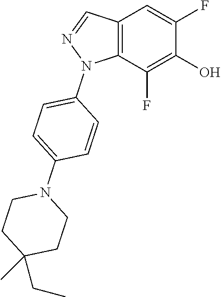 [SMILES: CCC1(C)CCN(c2ccc(-n3ncc4cc(F)c(O)c(F)c43)cc2)CC1] | 1-(4-(4-Ethyl-4-methylpiperidin-1- yl)phenyl)-5,7-difluoro-1H- indazol-6-ol | 372.2 |
| 24.6 | 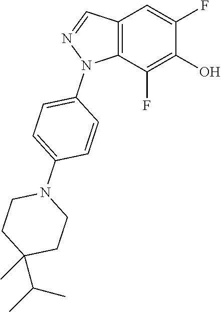 [SMILES: CC(C)C1(C)CCN(c2ccc(-n3ncc4cc(F)c(O)c(F)c43)cc2)CC1] | 5,7-Difluoro-1-(4-(4-isopropyl-4- methylpiperidin-1-yl)phenyl)-1H- indazol-6-ol | 386.3 |
| 24.7 | 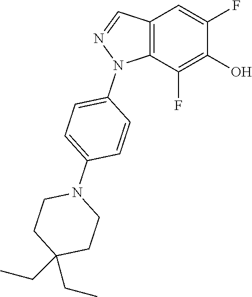 [SMILES: CCC1(CC)CCN(c2ccc(-n3ncc4cc(F)c(O)c(F)c43)cc2)CC1] | 1-(4-(4,4-Diethylpiperidin-1- yl)phenyl)-5,7-difluoro-1H- indazol-6-ol | 386.1 |
| 24.8 | 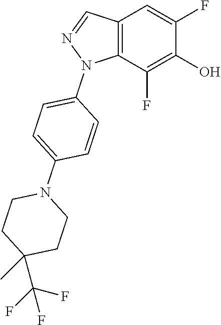 [SMILES: CC1(C(F)(F)F)CCN(c2ccc(-n3ncc4cc(F)c(O)c(F)c43)cc2)CC1] | 5,7-Difluoro-1-(4-(4-methyl-4- (trifluoromethyl)piperidin-1- yl)phenyl)-1H-indazol-6-ol | 412.0 |
| 24.9 | 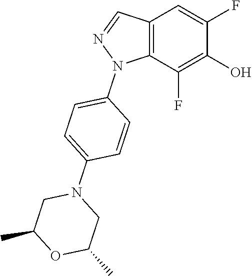 [SMILES: C[C@H]1CN(c2ccc(-n3ncc4cc(F)c(O)c(F)c43)cc2)C[C@H](C)O1] | 1-(4-((2S,6S)-2,6- Dimethylmorpholino)phenyl)-5,7- difluoro-1H-indazol-6-ol | 360.0 |
| 24.10 | 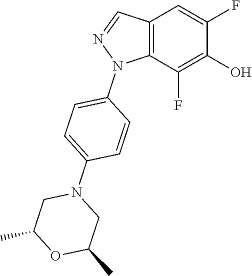 [SMILES: C[C@@H]1CN(c2ccc(-n3ncc4cc(F)c(O)c(F)c43)cc2)C[C@@H](C)O1] | 1-(4-((2R,6R)-2,6- Dimethylmorpholino)phenyl)-5,7- difluoro-1H-indazol-6-ol | 360.0 |
| 24.11 | 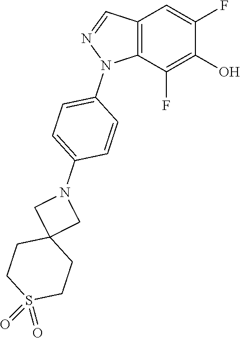 [SMILES: O=S1(=O)CCC2(CC1)CN(c1ccc(-n3ncc4cc(F)c(O)c(F)c43)cc1)C2] | 2-(4-(5,7-Difluoro-6-hydroxy-1H- indazol-1-yl)phenyl)-7-thia-2- azaspiro[3.5]nonane 7,7-dioxide | 420.0 |
| 24.12 | 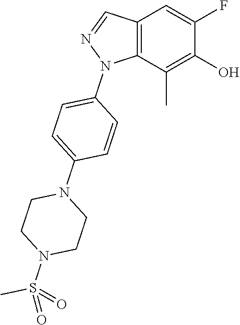 [SMILES: Cc1c(O)c(F)cc2cnn(-c3ccc(N4CCN(S(C)(=O)=O)CC4)cc3)c12] | 5-Fluoro-7-methyl-1-(4-(4- (methylsulfonyl)piperazin-1- yl)phenyl)-1H-indazol-6-ol | 405.4 |

Alternate conditions: Step 2: 35 min-2 h; HCl in Et₂O or 1M aq. HCl; Et₂O/CH₃OH or THF/CH₃OH; rt or 50° C.; 1.5-65 h.

### Compound 25

### 7-Chloro-5-fluoro-1-(4-(4-(methylsulfonyl)piperazin-1-yl)phenyl)-1H-indazol-6-ol

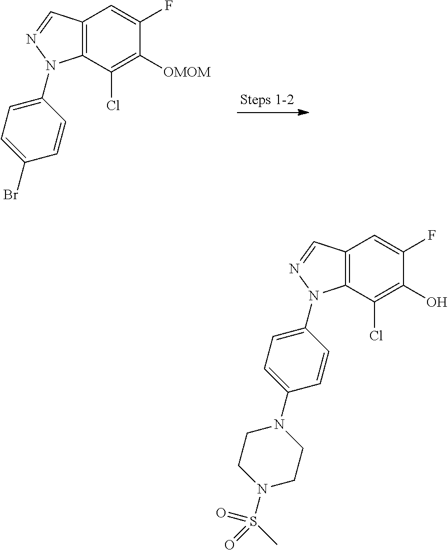

### Step 1: 7-Chloro-5-fluoro-6-(methoxymethoxy)-1-(4-(4-(methylsulfonyl)piperazin-1-yl)phenyl)-1H-indazole

Palladium(II) acetate (11.6 mg, 0.051 mmol) was added to a mixture of Intermediate 13.1 (200 mg, crude), 1-methylsulfonylpiperazine (256 mg, 1.56 mmol), t-BuONa (399 mg, 4.15 mmol), and tri-tert-butylphosphine (525 mg, 0.259 mol, 10% in toluene) in toluene (10 mL). The mixture was degassed with 3 vacuum/N₂ cycles, stirred at 80° C. for 0.5 h, allowed to cool to rt, poured into H₂O (20 mL), and then extracted with EtOAc (3×20 mL). The combined organic layers were washed with brine (2×30 mL), dried over Na₂SO₄, filtered, concentrated, and then purified by prep-HPLC [water(0.04% HCl)—CH₃CN] to give 7-chloro-5-fluoro-6-(methoxymethoxy)-1-(4-(4-(methylsulfonyl)piperazin-1-yl)phenyl)-1H-indazole (45 mg, 18%) as a white solid. ¹H NMR (400 MHz, DMSO-d₆): δ 8.30 (s, 1H), 7.79 (d, 1H), 7.36-7.34 (m, 2H), 7.09-7.06 (m, 2H), 5.19 (s, 2H), 3.52 (s, 3H), 3.38-3.35 (m, 4H), 3.28-3.26 (m, 4H), 2.94 (s, 3H); LCMS: 469.1 [M+H]⁺.

### Step 2: 7-Chloro-5-fluoro-1-(4-(4-(methylsulfonyl)piperazin-1-yl)phenyl)-1H-indazol-6-ol

Trifluoroacetic acid (770 mg, 6.75 mmol) was added to a solution of 7-chloro-5-fluoro-6-(methoxymethoxy)-1-(4-(4-(methylsulfonyl)piperazin-1-yl)phenyl)-1H-indazole (80 mg, 0.17 mmol) in CH₂Cl₂ (2 mL). The mixture was stirred at rt for 1 h, concentrated, and then purified by column chromatography (petroleum ether/ethyl acetate=5/1 to 1/1). The material was purified further by prep-HPLC [water(0.04% HCl)—CH₃CN] to give 7-chloro-5-fluoro-1-(4-(4-(methylsulfonyl)piperazin-1-yl)phenyl)-1H-indazol-6-ol (65 mg, 89%) as a white solid. ¹H NMR (400 MHz, DMSO-d₆): δ 10.59 (s, 1H), 8.18 (s, 1H), 7.66 (s, 1H), 7.32-7.30 (m, 2H), 7.07-7.05 (m, 2H), 3.37-3.33 (m, 4H), 3.29-3.26 (m, 4H), 2.94 (s, 3H); LCMS: 425.1 [M+H]⁺.

The Compounds below were synthesized from the appropriate Intermediate and the appropriate amine following the procedures described for Compound 25.

| Cmpd | Structure | Name | [M + H]⁺ |
|---|---|---|---|
| 25.1¹ | 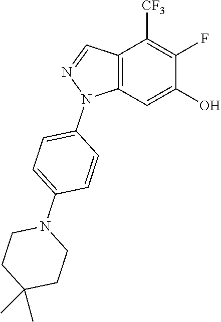 [SMILES: CC1(C)CCN(c2ccc(-n3ncc4c(C(F)(F)F)c(F)c(O)cc43)cc2)CC1] | 1-(4-(4,4-Dimethylpiperidin-1- yl)phenyl)-5-fluoro-4- (trifluoromethyl)-1H-indazol-6-ol | 408.2 |
| 25.2 | 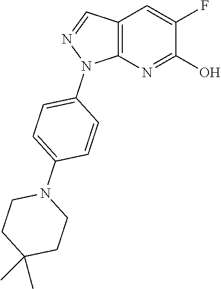 [SMILES: CC1(C)CCN(c2ccc(-n3ncc4cc(F)c(O)nc43)cc2)CC1] | 1-(4-(4,4-Dimethylpiperidin-1- yl)phenyl)-5-fluoro-1H- pyrazolo[3,4-b]pyridin-6-ol | 341.2 |
| 25.3 | 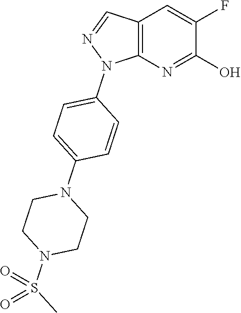 [SMILES: CS(=O)(=O)N1CCN(c2ccc(-n3ncc4cc(F)c(O)nc43)cc2)CC1] | 5-Fluoro-1-(4-(4- (methylsulfonyl)piperazin-1- yl)phenyl)-1H-pyrazolo[3,4- b]pyridin-6-ol | 392.0 |
| 25.4² | 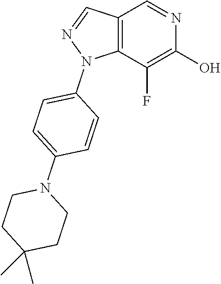 [SMILES: CC1(C)CCN(c2ccc(-n3ncc4cnc(O)c(F)c43)cc2)CC1] | 1-(4-(4,4-Dimethylpiperidin-1- yl)phenyl)-7-fluoro-1H- pyrazolo[4,3-c]pyridin-6-ol | 341.2 |
| 25.5² | 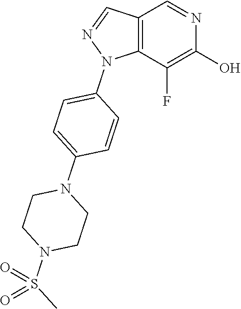 [SMILES: CS(=O)(=O)N1CCN(c2ccc(-n3ncc4cnc(O)c(F)c43)cc2)CC1] | 7-Fluoro-1-(4-(4- (methylsulfonyl)piperazin-1- yl)phenyl)-1H-pyrazolo[4,3- c]pyridin-6-ol | 392.0 |
| 25.6 | 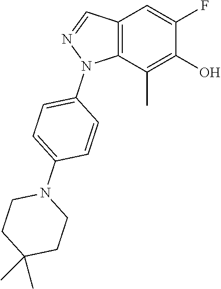 [SMILES: Cc1c(O)c(F)cc2cnn(-c3ccc(N4CCC(C)(C)CC4)cc3)c12] | 1-(4-(4,4-Dimethylpiperidin-1- yl)phenyl)-5-fluoro-7-methyl-1H- indazol-6-ol | 354.2 |

Alternate conditions: Step 1: Dioxane as solvent; 80-100° C.; 0.5-4 h; Amine was also an HCl salt;
¹Pd₂(dba)₃, XantPhos, t-BuONa, dioxane, 80° C., overnight.
²Demethylation: TMSCl, Nal, CH₃CN, reflux, 2 h.

### Compound 26

### 1-(6-(4,4-Dimethylpiperidin-1-yl)pyridin-3-yl)-5,7-difluoro-1H-indazol-6-ol

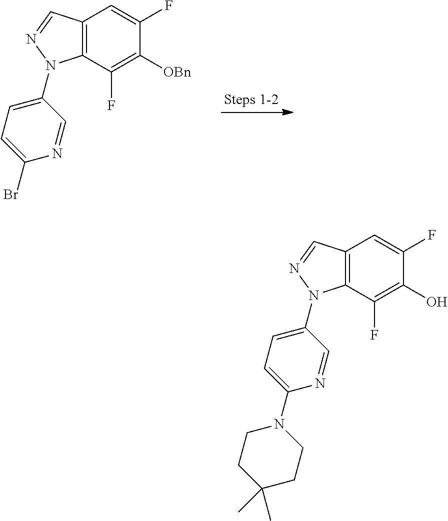 [SMILES: Fc1cc2cnn(-c3ccc(Br)nc3)c2c(F)c1OCc1ccccc1 ; CC1(C)CCN(c2ccc(-n3ncc4cc(F)c(O)c(F)c43)cn2)CC1]

### Step 1: 6-(Benzyloxy)-1-(6-(4,4-dimethylpiperidin-1-yl)pyridin-3-yl)-5,7-difluoro-1H-indazole

Pd₂(dba)₃ (22.0 mg, 0.024 mmol) and then t-Bu₃P (10% in toluene, 0.12 mL, 0.048 mmol) were added to a mixture of Intermediate 13.4 (100 mg, 0.240 mmol), 4,4-dimethylpiperidine HCl (43.2 mg, 0.288 mmol), t-BuONa (115 mg, 1.20 mmol), and toluene (2 mL) under N₂. The mixture was degassed with 3 vacuum/N₂ cycles, stirred at 100° C. for 1 h, cooled to rt, poured into H₂O (10 mL), and then extracted with EtOAc (3×10 mL). The combined organic layers were washed with brine (10 mL), dried over Na₂SO₄, filtered, concentrated, and then purified by silica gel chromatography (petroleum ether/ethyl acetate=50/1 to 20/1) to give 6-(benzyloxy)-1-(6-(4,4-dimethylpiperidin-1-yl)pyridin-3-yl)-5,7-difluoro-1H-indazole (100 mg, 92%) as a yellow solid. ¹H NMR (400 MHz, CDCl₃): δ 8.39-8.30 (m, 1H), 8.15-8.00 (m, 1H), 7.60 (d, 1H), 7.45 (d, 2H), 7.40-7.31 (m, 4H), 6.73 (d, 1H), 5.20 (s, 2H), 3.68-3.55 (m, 4H), 1.52-1.43 (m, 4H), 1.03 (s, 6H); LCMS: 449.3 [M+H]⁺.

### Step 2: 1-(6-(4,4-Dimethylpiperidin-1-yl)pyridin-3-yl)-5,7-difluoro-1H-indazol-6-ol

Palladium on carbon (10%, 30 mg, 0.028 mmol) was added to a solution of 6-(benzyloxy)-1-(6-(4,4-dimethylpiperidin-1-yl)pyridin-3-yl)-5,7-difluoro-1H-indazole (100 mg, 0.223 mmol) in CH₃OH (2 mL). The suspension was degassed with several vacuum/H₂ cycles, stirred under H₂ (15 psi) at rt for 2 h, and then filtered. The filtrate was concentrated and purified by reverse-phase HPLC [water (0.04% HCl)/CH₃CN] to give 1-(6-(4,4-dimethylpiperidin-1-yl)pyridin-3-yl)-5,7-difluoro-1H-indazol-6-ol (26 mg, 32%) as a white solid. ¹HNMR (400 MHz, DMSO-d₆): δ 10.57 (br s, 1H), 8.36-8.18 (m, 2H), 8.00-7.80 (m, 1H), 7.51 (d, 1H), 7.20-7.10 (m, 1H), 3.58-3.41 (m, 4H), 1.53-1.29 (m, 4H), 0.99 (s, 6H); LCMS: 359.0 [M+H]⁺.

The Compounds below were synthesized from the appropriate Intermediate and the appropriate amine following the procedures described for Compound 26.

| Cmpd | Structure | Name | [M + H]⁺ |
|---|---|---|---|
| 26.1 | 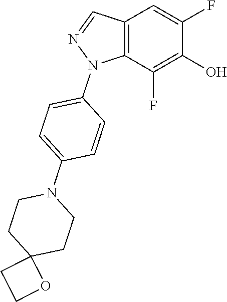 [SMILES: Oc1c(F)cc2cnn(-c3ccc(N4CCC5(CCO5)CC4)cc3)c2c1F] | 1-(4-(1-Oxa-7-azaspiro[3.5]nonan- 7-yl)phenyl)-5,7-difluoro-1H- indazol-6-ol | 372.3 |
| 26.2 | 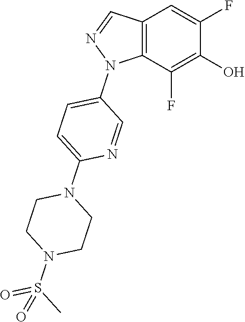 [SMILES: CS(=O)(=O)N1CCN(c2ccc(-n3ncc4cc(F)c(O)c(F)c43)cn2)CC1] | 5,7-Difluoro-1-(6-(4- (methylsulfonyl)piperazin-1- yl)pyridin-3-yl)-1H-indazol-6-ol | 410.0 |
| 26.3 | 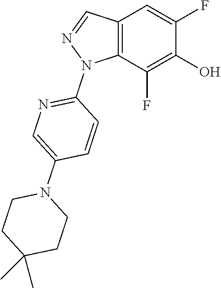 [SMILES: CC1(C)CCN(c2ccc(-n3ncc4cc(F)c(O)c(F)c43)nc2)CC1] | 1-(5-(4,4-Dimethylpiperidin-1- yl)pyridin-2-yl)-5,7-difluoro-1H- indazol-6-ol | 359.2 |
| 26.4 | 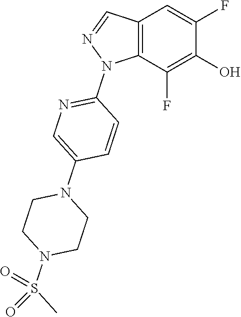 [SMILES: CS(=O)(=O)N1CCN(c2ccc(-n3ncc4cc(F)c(O)c(F)c43)nc2)CC1] | 5,7-Difluoro-1-(5-(4- (methylsulfonyl)piperazin-1- yl)pyridin-2-yl)-1H-indazol-6-ol | 410.0 |
| 26.5 | 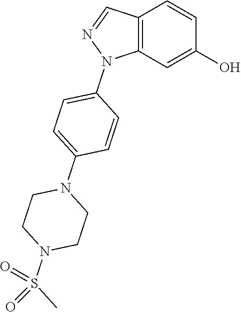 [SMILES: CS(=O)(=O)N1CCN(c2ccc(-n3ncc4ccc(O)cc43)cc2)CC1] | 1-(4-(4-(Methylsulfonyl)piperazin- 1-yl)phenyl)-1H-indazol-6-ol | 373.0 |
| 26.6 | 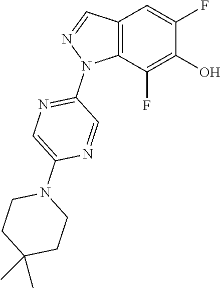 [SMILES: CC1(C)CCN(c2cnc(-n3ncc4cc(F)c(O)c(F)c43)cn2)CC1] | 1-(5-(4,4-Dimethylpiperidin-1- yl)pyrazin-2-yl)-5,7-difluoro-1H- indazol-6-ol | 360.2 |
| 26.7 | 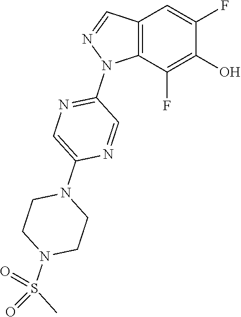 [SMILES: CS(=O)(=O)N1CCN(c2cnc(-n3ncc4cc(F)c(O)c(F)c43)cn2)CC1] | 5,7-Difluoro-1-(5-(4- (methylsulfonyl)piperazin-1- yl)pyrazin-2-yl)-1H-indazol-6-ol | 411.0 |
| 26.8 | 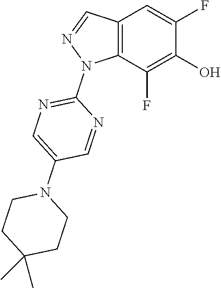 [SMILES: CC1(C)CCN(c2cnc(-n3ncc4cc(F)c(O)c(F)c43)nc2)CC1] | 1-(5-(4,4-Dimethylpiperidin-1- yl)pyrimidin-2-yl)-5,7-difluoro- 1H-indazol-6-ol | 360.3 |
| 26.9 | 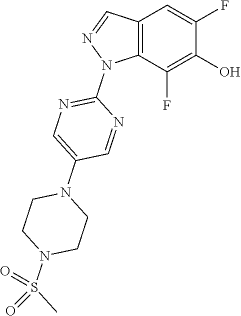 [SMILES: CS(=O)(=O)N1CCN(c2cnc(-n3ncc4cc(F)c(O)c(F)c43)nc2)CC1] | 5,7-Difluoro-1-(5-(4- (methylsulfonyl)piperazin-1- yl)pyrimidin-2-yl)-1H-indazol-6- ol | 411.0 |
| 26.10¹ | 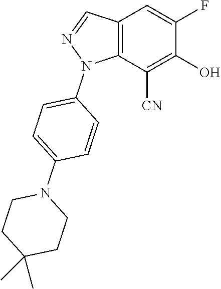 [SMILES: CC1(C)CCN(c2ccc(-n3ncc4cc(F)c(O)c(C#N)c43)cc2)CC1] | 1-(4-(4,4-Dimethylpiperidin-1- yl)phenyl)-5-fluoro-6-hydroxy- 1H-indazole-7-carbonitrile | 365.4 |
| 26.11¹ | 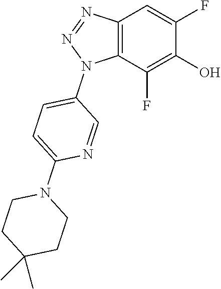 [SMILES: CC1(C)CCN(c2ccc(-n3nnc4cc(F)c(O)c(F)c43)cn2)CC1] | 1-(6-(4,4-Dimethylpiperidin-1- yl)pyridin-3-yl)-5,7-difluoro-1H- benzo[d][1,2,3]triazol-6-ol | 360.3 |
| 26.12¹ | 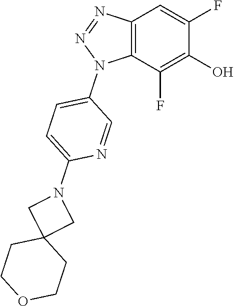 [SMILES: Oc1c(F)cc2nnn(-c3ccc(N4CC5(CCOCC5)C4)nc3)c2c1F] | 1-(6-(7-Oxa-2-azaspiro[3.5]nonan- 2-yl)pyridin-3-yl)-5,7-difluoro- 1H-benzo[d][1,2,3]triazol-6-ol | 374.4 |
| 26.13¹ | 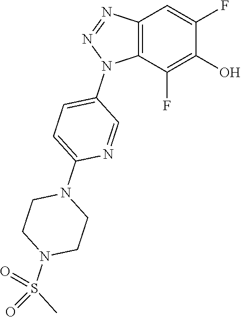 [SMILES: CS(=O)(=O)N1CCN(c2ccc(-n3nnc4cc(F)c(O)c(F)c43)cn2)CC1] | 5,7-Difluoro-1-(6-(4- (methylsulfonyl)piperazin-1- yl)pyridin-3-yl)-1H- benzo[d][1,2,3]triazol-6-ol | 411.3 |
| 26.14¹ | 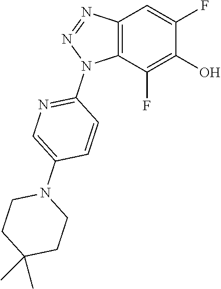 [SMILES: CC1(C)CCN(c2ccc(-n3nnc4cc(F)c(O)c(F)c43)nc2)CC1] | 1-(5-(4,4-Dimethylpiperidin-1- yl)pyridin-2-yl)-5,7-difluoro-1H- benzo[d][1,2,3]triazol-6-ol | 360.6 |
| 26.15¹ | 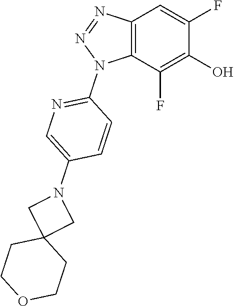 [SMILES: Oc1c(F)cc2nnn(-c3ccc(N4CC5(CCOCC5)C4)cn3)c2c1F] | 1-(5-(7-Oxa-2-azaspiro[3.5]nonan- 2-yl)pyridin-2-yl)-5,7-difluoro- 1H-benzo[d][1,2,3]triazol-6-ol | 374.3 |
| 26.16¹ | 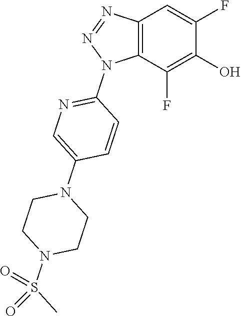 [SMILES: CS(=O)(=O)N1CCN(c2ccc(-n3nnc4cc(F)c(O)c(F)c43)nc2)CC1] | 5,7-Difluoro-1-(5-(4- (methylsulfonyl)piperazin-1- yl)pyridin-2-yl)-1//- benzo[d][1,2,3]triazol-6-ol | 411.3 |
| 26.17¹ | 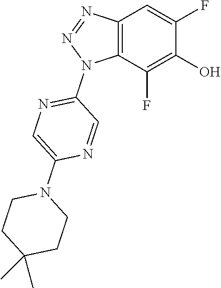 [SMILES: CC1(C)CCN(c2cnc(-n3nnc4cc(F)c(O)c(F)c43)cn2)CC1] | 1-(5-(4,4-Dimethylpiperidin-1- yl)pyrazin-2-yl)-5,7-difluoro-1H- benzo[d][1,2,3]triazol-6-ol | 361.3 |
| 26.18¹ | 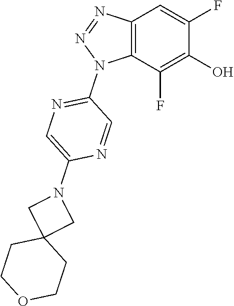 [SMILES: Oc1c(F)cc2nnn(-c3cnc(N4CC5(CCOCC5)C4)cn3)c2c1F] | 1-(5-(7-Oxa-2-azaspiro[3.5]nonan- 2-yl)pyrazin-2-yl)-5,7-difluoro- 1H-benzo[d][1,2,3]triazol-6-ol | 375.3 |
| 26.19¹ | 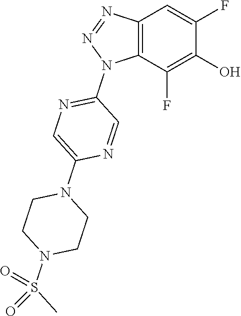 [SMILES: CS(=O)(=O)N1CCN(c2cnc(-n3nnc4cc(F)c(O)c(F)c43)cn2)CC1] | 5,7-Difluoro-1-(5-(4- (methylsulfonyl)piperazin-1- yl)pyrazin-2-yl)-1H- benzo[d][1,2,3]triazol-6-ol | 412.3 |
| 26.20¹ | 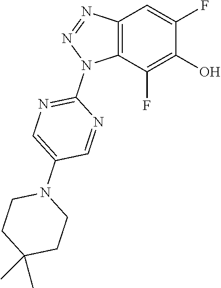 [SMILES: CC1(C)CCN(c2cnc(-n3nnc4cc(F)c(O)c(F)c43)nc2)CC1] | 1-(5-(4,4-Dimethylpiperidin-1- yl)pyrimidin-2-yl)-5,7-difluoro- 1H-benzo[d][1,2,3]triazol-6-ol | 361.1 |
| 26.21¹ | 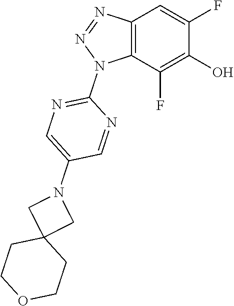 [SMILES: Oc1c(F)cc2nnn(-c3ncc(N4CC5(CCOCC5)C4)cn3)c2c1F] | 1-(5-(7-Oxa-2-azaspiro[3.5]nonan- 2-yl)pyrimidin-2-yl)-5,7-difluoro- 1H-benzo[d][1,2,3]triazol-6-ol | 375.0 |
| 26.22¹ | 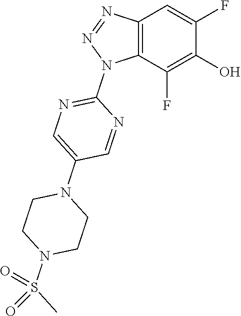 [SMILES: CS(=O)(=O)N1CCN(c2cnc(-n3nnc4cc(F)c(O)c(F)c43)nc2)CC1] | 5,7-Difluoro-1-(5-(4- (methylsulfonyl)piperazin-1- yl)pyrimidin-2-yl)-1H- benzo[d][1,2,3]triazol-6-ol | 412.0 |
| 26.23¹ | 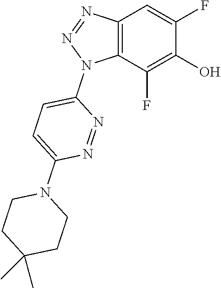 [SMILES: CC1(C)CCN(c2ccc(-n3nnc4cc(F)c(O)c(F)c43)nn2)CC1] | 1-(6-(4,4-Dimethylpiperidin-1- yl)pyridazin-3-yl)-5,7-difluoro- 1H-benzo[d][1,2,3]triazol-6-ol | 361.1 |
| 26.24¹ | 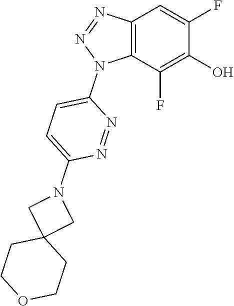 [SMILES: Oc1c(F)cc2nnn(-c3ccc(N4CC5(CCOCC5)C4)nn3)c2c1F] | 1-(6-(7-Oxa-2-azaspiro[3.5]nonan- 2-yl)pyridazin-3-yl)-5,7-difluoro- 1H-benzo[d][1,2,3]triazol-6-ol | 375.0 |
| 26.25¹ | 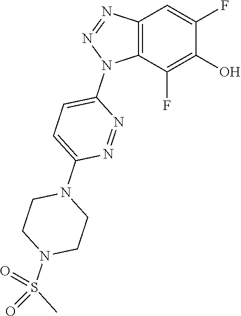 [SMILES: CS(=O)(=O)N1CCN(c2ccc(-n3nnc4cc(F)c(O)c(F)c43)nn2)CC1] | 5,7-Difluoro-1-(6-(4- (methylsulfonyl)piperazin-1- yl)pyridazin-3-yl)-1H- benzo[d][1,2,3]triazol-6-ol | 412.0 |
| 26.26² | 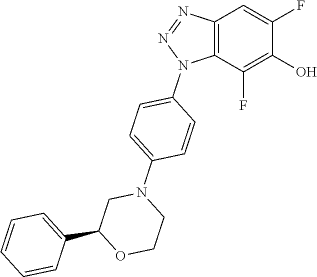 [SMILES: Oc1c(F)cc2nnn(-c3ccc(N4CCO[C@@H](c5ccccc5)C4)cc3)c2c1F] | (S)-5,7-Difluoro-1-(4-(2- phenylmorpholino)phenyl)-1H- benzo[d][1,2,3]triazol-6-ol | 409.0 |
| 26.27² | 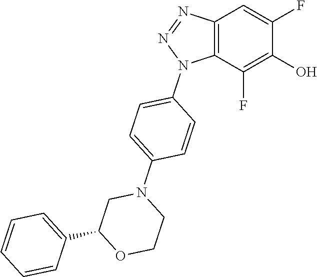 [SMILES: Oc1c(F)cc2nnn(-c3ccc(N4CCO[C@H](c5ccccc5)C4)cc3)c2c1F] | (R)-5,7-Difluoro-1-(4-(2- phenylmorpholino)phenyl)-1H- benzo[d][1,2,3]triazol-6-ol | 409.0 |
| 26.28² | 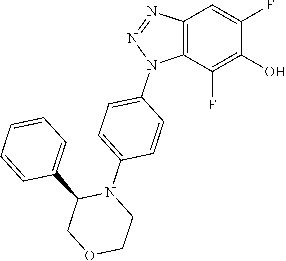 [SMILES: Oc1c(F)cc2nnn(-c3ccc(N4CCOC[C@H]4c4ccccc4)cc3)c2c1F] | (R)-5,7-Difluoro-1-(4-(3- phenylmorpholino)phenyl)-1H- benzo[d][1,2,3]triazol-6-ol | 409.0 |
| 26.29² | 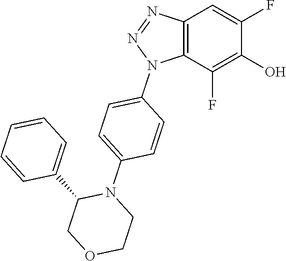 [SMILES: Oc1c(F)cc2nnn(-c3ccc(N4CCOC[C@@H]4c4ccccc4)cc3)c2c1F] | (S)-5,7-Difluoro-1-(4-(3- phenylmorpholino)phenyl)-1H- benzo[d][1,2,3]triazol-6-ol | 409.1 |

Alternate conditions:
Step 1: Amine was also a free base; In some instances, t-BuONa was dried under high vacuum by heating with a heat gun for a few minutes and then allowing to cool prior to weighing;
Step 1: Pd₂(dba)₃, RuPhos, t-BuONa or Cs₂CO₃, toluene or dioxane, 80-100° C., 1-16 h or Pd(t-Bu₃P)₂, t-BuONa, toluene, 100° C., 1 h or Pd₂(dba)₃, XantPhos, Cs₂CO₃, dioxane, 80-100° C., 1 h-ON or Cs₂CO₃, DMF, rt-100° C., 4-24 h. Step 2: CH₃OH, EtOAc, THF, or combination thereof, 2-71 h;
¹Step 2: TFA, 50 °C, 1-8 h.
²Benzyl was cleaved during Step 1 conditions.

### Compound 27

### 1-(4-(4,4-Dimethylcyclohexyl)phenyl)-5,7-difluoro-1H-indazol-6-ol

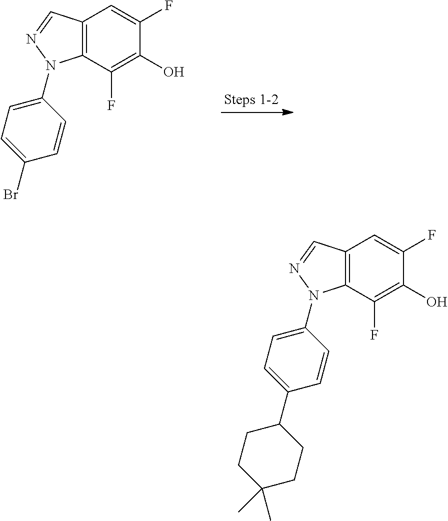 [SMILES: Oc1c(F)cc2cnn(-c3ccc(Br)cc3)c2c1F ; CC1(C)CCC(c2ccc(-n3ncc4cc(F)c(O)c(F)c43)cc2)CC1]

### Step 1: 1-(4′,4′-Dimethyl-2′,3′,4′,5′-tetrahydro-[1,1′-biphenyl]-4-yl)-5,7-difluoro-1H-indazol-6-ol

A mixture of Intermediate 22 (45 mg, 0.14 mmol), 4,4-dimethylcyclohexen-1-ylboronic acid (32 mg, 0.21 mmol), 1,1′-bis(diphenylphosphino)ferrocene dichloropalladium(II) (10 mg, 0.014 mmol), DME (1 mL), water (0.35 mL), and aqueous potassium carbonate (3 M, 138 μL, 0.415 mmol) was stirred at 80° C. for 2 hand allowed to cool to rt. The aqueous layer was pipetted off, and the remaining mixture was diluted with 10 mL EtOAc. The organics were dried (Na₂SO₄), filtered, concentrated, and then purified by silica gel chromatography (0-20% EtOAc in hexanes) to give 1-(4′,4′-dimethyl-2′,3′,4′,5′-tetrahydro-[1,1′-biphenyl]-4-yl)-5,7-difluoro-1H-indazol-6-ol (30 mg, 61%) as a white solid. ¹H NMR (400 MHz, DMSO-d₆): δ 10.49 (s, 1H), 8.21 (d, J=2.2 Hz, 1H), 7.54-7.49 (m, 2H), 7.48-7.43 (m, 3H), 6.19-6.13 (m, 1H), 2.40-2.35 (m, 2H), 1.98-1.92 (m, 2H), 1.45 (t, J=6.4 Hz, 2H), 0.89 (s, 6H) LCMS: 355.0 [M+H]⁺

### Step 2: 1-(4-(4,4-Dimethylcyclohexyl)phenyl)-5,7-difluoro-1H-indazol-6-ol

Palladium on carbon (5 wt. %) was added to a mixture of 1-(4′,4′-dimethyl-2′,3′,4′,5′-tetrahydro-[1,1′-biphenyl]-4-yl)-5,7-difluoro-1H-indazol-6-ol (25 mg, 0.07 mmol), and CH₃OH (1.5 mL) at rt under N₂. The mixture was degassed with 3 vacuum/H₂ cycles, stirred at rt under H₂ for 93 h, and then filtered through Celite. The filter cake was washed with CH₃OH (5 mL), and the filtrate was concentrated to give 1-(4′-hydroxy-[1,1′-biphenyl]-4-yl)-1H-indazol-6-ol (28 mg) as a dark beige solid. ¹H NMR (400 MHz, DMSO-d₆): δ 11.28-10.52 (m, 1H), 8.21 (d, J=2.1 Hz, 1H), 7.50-7.43 (m, 3H), 7.41-7.36 (m, 2H), 2.49-2.45 (m, 1H), 1.70-1.58 (m, 4H), 1.51-1.44 (m, 2H), 1.39-1.29 (m, 2H), 0.99 (s, 3H), 0.97-0.93 (m, 3H); LCMS: 357.0 [M+H]⁺.

The Compounds below were synthesized from Intermediate 22 or Intermediate 22.1 and the appropriate boronic acid/ester following the procedures described for Compound 27.

| Cmpd | Structure | Name | [M + H]⁺ |
|---|---|---|---|
| 27.1¹ | 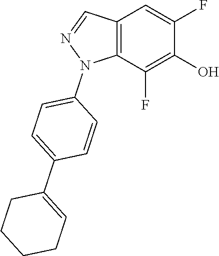 [SMILES: Oc1c(F)cc2cnn(-c3ccc(C4=CCCCC4)cc3)c2c1F] | 5,7-Difluoro-1-(2′,3′,4′,5′- tetrahydro-[1,1′-biphenyl]-4-yl)- 1H-indazol-6-ol | 326.8 |
| 27.2¹ | 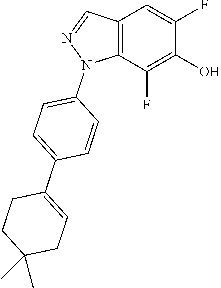 [SMILES: CC1(C)CC=C(c2ccc(-n3ncc4cc(F)c(O)c(F)c43)cc2)CC1] | 1-(4′,4′-Dimethyl-2′,3′,4′,5′- tetrahydro-[1,1′-biphenyl]-4-yl)- 5,7-difluoro-1H-indazol-6-ol | 355.0 |
| 27.3¹ | 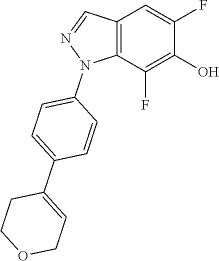 [SMILES: Oc1c(F)cc2cnn(-c3ccc(C4=CCOCC4)cc3)c2c1F] | 1-(4-(3,6-Dihydro-2H-pyran-4- yl)phenyl)-5,7-difluoro-1H- indazol-6-ol | 328.9 |
| 27.4¹ | 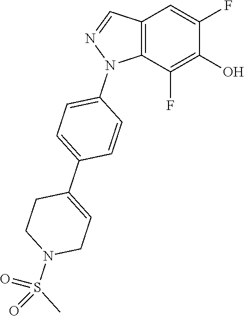 [SMILES: CS(=O)(=O)N1CC=C(c2ccc(-n3ncc4cc(F)c(O)c(F)c43)cc2)CC1] | 5,7-Difluoro-1-(4-(1- (methylsulfonyl)-1,2,3,6- tetrahydropyridin-4-yl)phenyl)- 1H-indazol-6-ol | 405.9 |
| 27.5 | 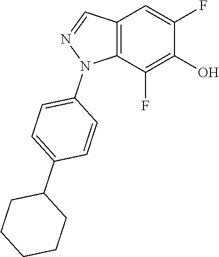 [SMILES: Oc1c(F)cc2cnn(-c3ccc(C4CCCCC4)cc3)c2c1F] | 1-(4-Cyclohexylphenyl)-5,7- difluoro-1H-indazol-6-ol | 328.9 |
| 27.6 | 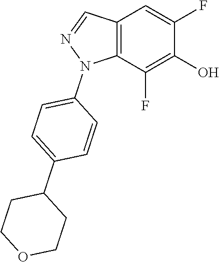 [SMILES: Oc1c(F)cc2cnn(-c3ccc(C4CCOCC4)cc3)c2c1F] | 5,7-Difluoro-1-(4-(tetrahydro-2H- pyran-4-yl)phenyl)-1H-indazol-6-ol | 330.9 |
| 27.7 | 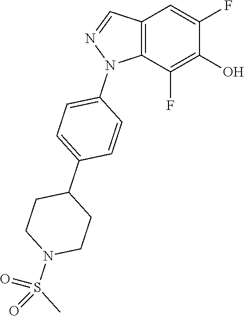 [SMILES: CS(=O)(=O)N1CCC(c2ccc(-n3ncc4cc(F)c(O)c(F)c43)cc2)CC1] | 5,7-Difluoro-1-(4-(1- (methylsulfonyl)piperidin-4- yl)phenyl)-1H-indazol-6-ol | 407.9 |
| 27.8 | 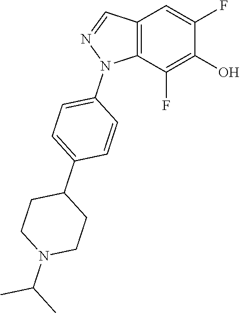 [SMILES: CC(C)N1CCC(c2ccc(-n3ncc4cc(F)c(O)c(F)c43)cc2)CC1] | 5,7-Difluoro-1-(4-(1- isopropylpiperidin-4-yl)phenyl)- 1H-indazol-6-ol | 372.1 |
| 27.9 | 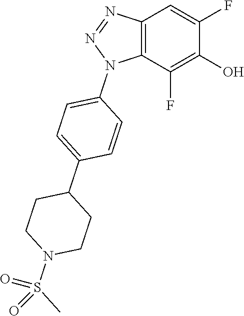 [SMILES: CS(=O)(=O)N1CCC(c2ccc(-n3nnc4cc(F)c(O)c(F)c43)cc2)CC1] | 5,7-Difluoro-1-(4-(1- (methylsulfonyl)piperidin-4- yl)phenyl)-1H- benzo[d][1,2,3]triazol-6-ol | 409.0 |
| 27.10 | 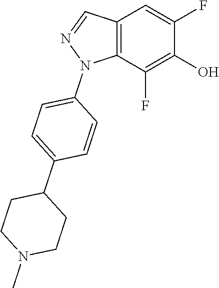 [SMILES: CN1CCC(c2ccc(-n3ncc4cc(F)c(O)c(F)c43)cc2)CC1] | 5,7-Difluoro-1-(4-(1- methylpiperidin-4-yl)phenyl)-1H- indazol-6-ol | 344.0 |

Alternate conditions: Step 1: 0.5-28 h. Step 2: 17-93 h; THF as cosolvent.
¹Step 1 only.

### Compound 28

### 5,7-Difluoro-1-(4-(1-(methylsulfonyl)piperidin-4-yl)phenyl)-1H-benzo[d]imidazol-6-ol

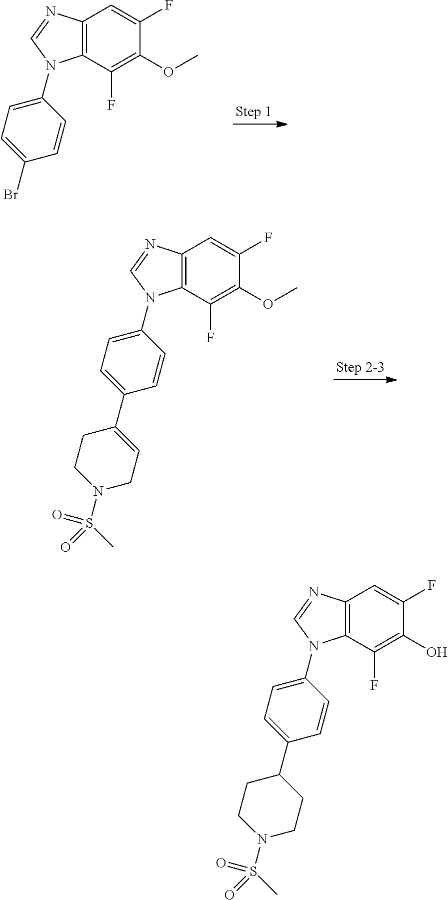 [SMILES: COc1c(F)cc2ncn(-c3ccc(Br)cc3)c2c1F ; COc1c(F)cc2ncn(-c3ccc(C4=CCN(S(C)(=O)=O)CC4)cc3)c2c1F ; CS(=O)(=O)N1CCC(c2ccc(-n3cnc4cc(F)c(O)c(F)c43)cc2)CC1]

### Step 1: 5,7-Difluoro-6-methoxy-1-(4-(1-(methylsulfonyl)-1,2,3,6-tetrahydropyridin-4-yl)phenyl)-1H-benzo[d]imidazole

A mixture of Intermediate 16 (60 mg, 0.18 mmol), 1-(methylsulfonyl)-4-(4,4,5,5-tetramethyl(1,3,2-dioxaborolan-2-yl))-1,2,5,6-tetrahydropyridine (287 mg, 0.27 mmol), 1,1′-bis(diphenylphosphino)ferrocene dichloropalladium(II) (13 mg, 0.018 mmol), potassium carbonate (3.0 M aq soln, 177 μL, 0.53 mmol), 1,2-dimethoxyethane (1 mL), and water (350 μL) was heated at 80° C. for 1 h. The mixture was cooled to rt, and the aqueous layer was pipetted off. The remaining mixture was diluted with EtOAc (10 mL). The organic layer was dried (Na₂SO₄), filtered, concentrated, and then purified by silica gel chromatography (0-25% EtOAc in CH₂Cl₂) to give 5,7-difluoro-6-methoxy-1-(4-(1-(methylsulfonyl)-1,2,3,6-tetrahydropyridin-4-yl)phenyl)-1H-benzo[d]imidazole (60 mg, 81%) as a pink solid. ¹H NMR (400 MHz, DMSO-d₆): δ 8.51 (s, 1H), 7.69-7.62 (m, 4H), 7.62-7.58 (m, 1H), 6.35 (br s, 1H), 3.92 (s, 5H), 3.42 (t, J=5.6 Hz, 2H), 2.97 (s, 3H), 2.68 (br s, 2H); LCMS: 420.4 [M+H]⁺.

### Step 2: 5,7-Difluoro-1-(4-(1-(methylsulfonyl)-1,2,3,6-tetrahydropyridin-4-yl)phenyl)-1H-benzo[d]imidazol-6-ol

Boron tribromide (1 M in CH₂Cl₂ 543 μL, 0.54 mmol) was added slowly to a mixture of 5,7-difluoro-6-methoxy-1-(4-(1-(methylsulfonyl)-1,2,3,6-tetrahydropyridin-4-yl)phenyl)-1H-benzo[d]imidazole (57 mg, 0.14 mmol) in CH₂Cl₂ (0.9 mL) at 0° C. under N₂. The mixture was stirred 0° C. to rt over 2 h, cooled in an ice/water bath, and then quenched slowly with CH₃OH (1 mL). The mixture was concentrated and purified by silica gel chromatography (0-10% CH₃OH in CH₂Cl₂). The product fractions were combined, concentrated, and then triturated with EtOAc:CH₂Cl₂ (1:1, 2 mL) and minimal CH₃OH. The solids were filtered and washed with CH₂Cl₂ (2 mL) to give 5,7-difluoro-1-(4-(1-(methylsulfonyl)-1,2,3,6-tetrahydropyridin-4-yl)phenyl)-1H-benzo[d]imidazol-6-ol (52 mg, 94%) as a beige solid. ¹H NMR (400 MHz, DMSO-d₆): δ 10.23-9.91 (m, 1H), 8.46 (br s, 1H), 7.69-7.60 (m, 4H), 7.50 (d, J=10.4 Hz, 1H), 6.35 (br d, J=1.0 Hz, 1H), 3.91 (br s, 2H), 3.42 (t, J=5.6 Hz, 2H), 2.97 (s, 3H), 2.72-2.66 (m, 2H); LCMS: 406.1 [M+H]⁺.

### Step 3: 5,7-Difluoro-1-(4-(1-(methylsulfonyl)piperidin-4-yl)phenyl)-1H-benzo[d]imidazol-6-ol

Palladium on carbon (10 wt. %) was added to a mixture of 5,7-difluoro-1-(4-(1-(methylsulfonyl)-1,2,3,6-tetrahydropyridin-4-yl)phenyl)-1H-benzo[d]imidazol-6-ol (49 mg, 0.12 mmol) and CH₃OH (4 mL) at rt under N₂. The mixture was degassed with 3 vacuum/H₂ cycles, stirred at rt for 17 h, and then filtered through Celite. The filter cake was washed with CH₃OH (3 mL), and the filtrate was concentrated. The crude product was purified by silica gel chromatography (0-20% CH₃OH in CH₂Cl₂). The product fractions were combined, concentrated, and then sonicated with minimal EtOAc:CH₂Cl₂. The solids were filtered and washed with CH₂Cl₂ (2 mL) to give 5,7-difluoro-1-(4-(1-(methylsulfonyl)piperidin-4-yl)phenyl)-1H-benzo[d]imidazol-6-ol (20 mg, 41%) as a light purple solid. ¹H NMR (400 MHz, DMSO-d₆): δ 9.97 (s, 1H), 8.34 (s, 1H), 7.58-7.52 (m, 2H), 7.51-7.44 (m, 3H), 3.71 (br d, J=11.4 Hz, 2H), 2.92 (s, 3H), 2.85 (br t, J=11.5 Hz, 2H), 2.81-2.72 (m, 1H), 1.94 (br d, J=13.1 Hz, 2H), 1.81-1.69 (m, 2H); LCMS: 408.0 [M+H]⁺.

### Compound 29

### 1-(4-(1-Ethylpiperidin-4-yl)phenyl)-5,7-difluoro-1H-indazol-6-ol

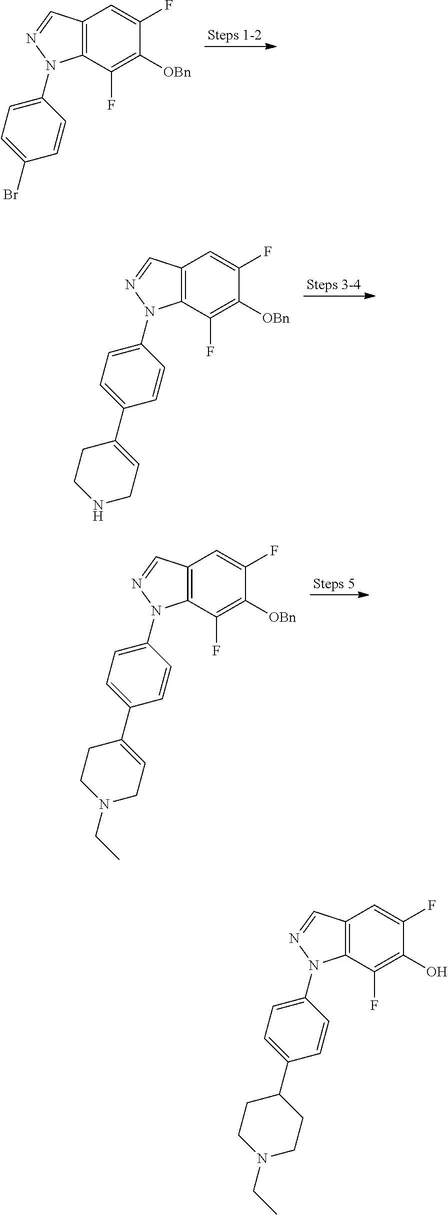 [SMILES: Fc1cc2cnn(-c3ccc(Br)cc3)c2c(F)c1OCc1ccccc1 ; Fc1cc2cnn(-c3ccc(C4=CCNCC4)cc3)c2c(F)c1OCc1ccccc1 ; CCN1CC=C(c2ccc(-n3ncc4cc(F)c(OCc5ccccc5)c(F)c43)cc2)CC1 ; CCN1CCC(c2ccc(-n3ncc4cc(F)c(O)c(F)c43)cc2)CC1]

### Step 1: tert-Butyl 4-(4-(6-(benzyloxy)-5,7-difluoro-1H-indazol-1-yl)phenyl)-5,6-dihydropyridine-1(2H)-carboxylate

A mixture of Intermediate 14.9 (100 mg, 0.24 mmol), tert-butyl 4-(4,4,5,5-tetramethyl-1,3,2-dioxaborolan-2-yl)-1,2,5,6-tetrahydropyridinecarboxylate (111 mg, 0.36 mmol), 1,1′-bis(diphenylphosphino)ferrocene dichloropalladium(II) (19 mg, 0.026 mmol), 3.0 M potassium carbonate (243 μL, 0.73 mmol), 1,2-dimethoxyethane (2 mL), and water (720 μL) was heated at 80° C. for 2.5 h and cooled to rt. The aqueous layer was pipetted off, and the remaining mixture was diluted with EtOAc (10 mL). The organic layer was washed with brine (10 mL), dried (Na₂SO₄), filtered, concentrated, and then purified by silica gel chromatography (0-5% EtOAc in CH₂Cl₂) to give tert-butyl 4-(4-(6-(benzyloxy)-5,7-difluoro-1H-indazol-1-yl)phenyl)-5,6-dihydropyridine-1(2H)-carboxylate (127 mg, 90%) as a yellow liquid. ¹HNMR (400 MHz, DMSO-d₆): δ 8.40 (s, 1H), 7.68-7.56 (m, 5H), 7.47-7.42 (m, 2H), 7.42-7.34 (m, 3H), 6.29 (br s, 1H), 5.22 (s, 2H), 4.04 (br s, 2H), 3.62-3.53 (m, 2H), 2.54 (br s, 2H), 1.45 (s, 9H); LCMS: 518.5 [M+H]⁺.

### Step 2: 6-(Benzyloxy)-5,7-difluoro-1-(4-(1,2,3,6-tetrahydropyridin-4-yl)phenyl)-1H-indazole

Trifluoroacetic acid (0.4 mL) was added to tert-butyl 4-(4-(6-(benzyloxy)-5,7-difluoro-1H-indazol-1-yl)phenyl)-5,6-dihydropyridine-1(2H)-carboxylate (124 mg, 0.21 mmol) in CH₂Cl₂ (1.6 mL). The mixture was stirred at rt for 30 min, concentrated, and then diluted with CH₂Cl₂ (10 mL). The organic layer was washed with sat'd NaHCO₃ (2×10 mL), dried (Na₂SO₄), filtered, and then concentrated to give 6-(benzyloxy)-5,7-difluoro-1-(4-(1,2,3,6-tetrahydropyridin-4-yl)phenyl)-1H-indazole (83 mg, 90%) as a beige solid. ¹H NMR (400 MHz, DMSO-d₆): δ 8.39 (s, 1H), 7.65 (d, J=9.5 Hz, 1H), 7.62-7.53 (m, 4H), 7.47-7.42 (m, 2H), 7.42-7.34 (m, 3H), 6.34 (br s, 1H), 5.22 (s, 2H), 3.42 (br s, 2H), 2.96 (t, J=5.5 Hz, 2H), 2.88-2.67 (m, 1H), 2.41 (br s, 2H); LCMS: 418.2 [M+H]⁺.

### Step 3: 1-(4-(4-(6-(Benzyloxy)-5,7-difluoro-1H-indazol-1-yl)phenyl)-5,6-dihydropyridin-1(2H)-yl)ethanone

Acetyl chloride (21 μL, 0.30 mmol) was added to a solution of 6-(benzyloxy)-5,7-difluoro-1-(4-(1,2,3,6-tetrahydropyridin-4-yl)phenyl)-1H-indazole (80 mg, 0.18 mmol) and triethylamine (70 μL, 0.50 mmol) in CH₂Cl₂ (2 mL) at rt. The mixture was stirred at rt over 2 h, concentrated, and then purified by silica gel chromatography (0-100% EtOAc in CH₂Cl₂) to give 1-(4-(4-(6-(benzyloxy)-5,7-difluoro-1H-indazol-1-yl)phenyl)-5,6-dihydropyridin-1(2H)-yl)ethenone (77 mg, 92%) as an off-white solid. ¹H NMR (400 MHz, DMSO-d₆): δ 8.40 (s, 1H), 7.68-7.56 (m, 5H), 7.48-7.43 (m, 2H), 7.42-7.34 (m, 3H), 6.31 (br s, 1H), 5.22 (s, 2H), 4.23-4.11 (m, 2H), 3.68 (td, J=5.6, 11.1 Hz, 2H), 2.62 (br s, 1H), 2.56-2.52 (m, 1H), 2.08 (d, J=15.2 Hz, 3H); LCMS: 460.1 [M+H]⁺.

### Step 4: 6-(Benzyloxy)-1-(4-(1-ethyl-1,2,3,6-tetrahydropyridin-4-yl)phenyl)-5,7-difluoro-1H-indazole TFA salt

Lithium aluminum hydride solution (2.0 M in THF, 160 μL, 0.32 mmol) was added to a solution of 1-(4-(4-(6-(benzyloxy)-5,7-difluoro-1H-indazol-1-yl)phenyl)-5,6-dihydropyridin-1(2H)-yl)ethanone (74 mg, 0.16 mmol) in THF (1 mL) at 0° C. The mixture was stirred at 0° C. to rt over 3 h, cooled in ice/water bath, quenched with water (1 mL), diluted with 1 M NaOH (1 mL) and EtOAc (10 mL), and then filtered through Celite. The layers from the filtrate were separated. The organic layer was washed with brine (10 mL), dried (Na₂SO₄), filtered, concentrated, and then purified by silica gel chromatography (0-100% EtOAc in CH₂Cl₂ then 0-10% CH₃OH in CH₂C₂). The product was purified further by reverse-phase HPLC (10-65% CH₃CN in water with 0.1% TFA) to give 6-(benzyloxy)-1-(4-(1-ethyl-1,2,3,6-tetrahydropyridin-4-yl)phenyl)-5,7-difluoro-1H-indazole TFA salt (42 mg, 47%) as a yellow liquid. ¹H NMR (400 MHz, DMSO-d₆): δ 9.68-9.58 (m, 1H), 8.42 (s, 1H), 7.80-7.70 (m, 2H), 7.69 (s, 1H), 7.66 (br s, 1H), 7.52 (br d, J=7.2 Hz, 1H), 7.48-7.42 (m, 2H), 7.39 (q, J=6.8 Hz, 2H), 6.34 (br s, 1H), 5.23 (s, 2H), 4.07 (br d, J=17.0 Hz, 1H), 3.89-3.78 (m, ¹H), 3.78-3.70 (m, ¹H), 3.31-3.22 (m, 3H), 2.87 (br s, 2H), 1.31 (t, J=7.3 Hz, 3H); LCMS: 446.0 [M+H]⁺.

### Step 5: 1-(4-(1-Ethylpiperidin-4-yl)phenyl)-5,7-difluoro-1H-indazol-6-ol TFA salt

Palladium on carbon (10 wt. %) was added to a mixture of 6-(benzyloxy)-1-(4-(1-ethyl-1,2,3,6-tetrahydropyridin-4-yl)phenyl)-5,7-difluoro-1H-indazole (39 mg, 0.07 mmol), 1.25 M hydrochloric acid in methanol (140 μL, 0.18 mmol), and CH₃OH (2 mL) at rt under N₂. The mixture was degassed with 3 vacuum/H₂ cycles, stirred at rt for 17 h, and then filtered. The filter cake was washed with CH₃OH (3 mL). The filtrate was concentrated and then purified by reverse-phase HPLC (27% CH₃CN in water with 0.1% TFA) to give 1-(4-(1-ethylpiperidin-4-yl)phenyl)-5,7-difluoro-1H-indazol-6-ol TFA salt (6.8 mg, 27%) as a white sticky solid. ¹HNMR (400 MHz, DMSO-d₆): δ 10.57 (s, 1H), 8.29 (s, 1H), 7.61-7.51 (m, 3H), 7.42 (d, J=8.3 Hz, 2H), 3.62 (br d, J=11.9 Hz, 2H), 3.22-3.14 (m, 2H), 3.12-3.00 (m, 2H), 3.00-2.89 (m, 1H), 2.12 (br d, J=13.9 Hz, 2H), 1.88 (br d, J=12.8 Hz, 2H), 1.27 (t, J=7.3 Hz, 3H); LCMS: 358.1 [M+H]⁺.

### Compound 30

### 5,7-Difluoro-1-(4-(1-(methylsulfonyl)azetidin-3-yl)phenyl)-1H-indazol-6-ol

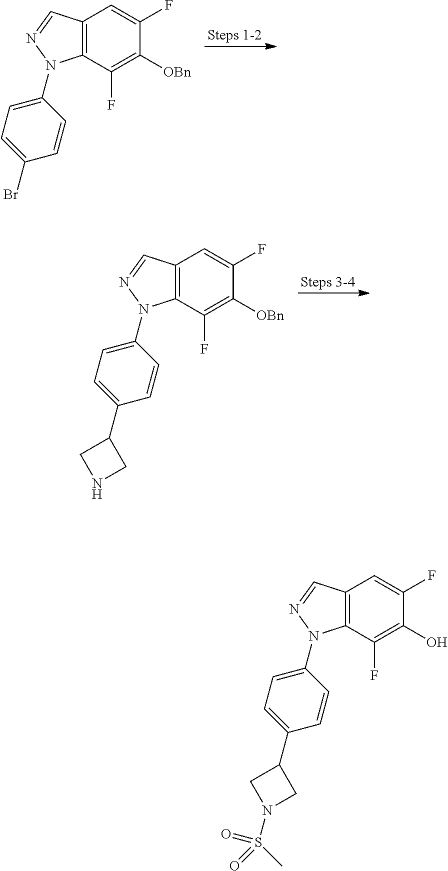 [SMILES: Fc1cc2cnn(-c3ccc(Br)cc3)c2c(F)c1OCc1ccccc1 ; Fc1cc2cnn(-c3ccc(C4CNC4)cc3)c2c(F)c1OCc1ccccc1 ; CS(=O)(=O)N1CC(c2ccc(-n3ncc4cc(F)c(O)c(F)c43)cc2)C1]

### Step 1: tert-Butyl 3-(4-(6-(benzyloxy)-5,7-difluoro-LH-indazol-1-yl)phenyl)azetidine-1-carboxylate

tert-Butyl 3-bromoazetidine-1-carboxylate (157 μL, 0.96 mmol) in DMA (0.2 mL)* was added to a mixture of NiCl₂ glyme (10 mg, 0.05 mmol), sodium iodide (37 mg, 0.25 mmol), BBBPY (19 mg, 0.07 mmol), zinc powder (127 mg, 1.94 mmol) and DMA (1 mL)*. Intermediate 14.9 (200 mg, 0.48 mmol) in DMA (0.8 mL)* and then trifluoroacetic acid (4.6 μL, 0.06 mmol) were added. The reaction was stirred at 60° C. for 23 h, cooled to rt, diluted with EtOAc (3 mL), and then filtered. The filter cake was washed with EtOAc (10 mL). The filtrate was washed with brine (10 mL), dried (Na₂SO₄), filtered, concentrated, and then purified by silica gel chromatography (0-40% EtOAc in hexanes) to give tert-butyl 3-(4-(6-(benzyloxy)-5,7-difluoro-1H-indazol-1-yl)phenyl)azetidine-1-carboxylate (134 mg, 55%) as a sticky white solid. ¹H NMR (400 MHz, DMSO-d₆): δ 8.39 (s, 1H), 7.65 (d, J=9.7 Hz, 1H), 7.61-7.56 (m, 2H), 7.55-7.50 (m, 2H), 7.47-7.43 (m, 2H), 7.43-7.34 (m, 3H), 5.22 (s, 2H), 4.30 (br s, 2H), 3.91 (br s, 3H), 1.42 (s, 9H); LCMS: 392.2 [(M-Boc+H)+H]⁺.

* The vials were degassed and backfilled with N₂ prior to combining.

### Step 2: 1-(4-(Azetidin-3-yl)phenyl)-6-(benzyloxy)-5,7-difluoro-1H-indazole

Trifluoroacetic acid (200 μL) was added to tert-butyl 3-(4-(6-(benzyloxy)-5,7-difluoro-1H-indazol-1-yl)phenyl)azetidine-1-carboxylate (131 mg, 0.26 mmol) in CH₂Cl₂ (1 mL). The mixture was stirred at rt for 1 h, concentrated, and then diluted with CH₂Cl₂ (10 mL). The organic layer was washed with sat'd NaHCO₃ (2×10 mL), dried (Na₂SO₄), filtered, concentrated, and then purified by silica gel chromatography (0-100% EtOAc in CH₂Cl₂ then 0-20% MeOH in CH₂Cl₂) to give 1-(4-(azetidin-3-yl)phenyl)-6-(benzyloxy)-5,7-difluoro-1H-indazole (77 mg, 76%) as a sticky yellow solid. ¹H NMR (400 MHz, DMSO-d₆): δ 8.38 (s, 1H), 7.65 (d, J=9.9 Hz, 1H), 7.60-7.51 (m, 4H), 7.47-7.42 (m, 2H), 7.42-7.34 (m, 3H), 5.22 (s, 2H), 4.03-3.95 (m, 1H), 3.91 (brt, J=7.7 Hz, 2H), 3.74 (brt, J=7.3 Hz, 2H), 3.25 (br dd, J=5.4, 6.7 Hz, 1H); LCMS: 392.2 [M+H]⁺.

### Step 3: 6-(Benzyloxy)-5,7-difluoro-1-(4-(1-(methylsulfonyl)azetidin-3-yl)phenyl)-1H-indazole

Methanesulfonyl chloride (16 μL, 0.21 mmol) was added to a solution of 1-(4-(azetidin-3-yl)phenyl)-6-(benzyloxy)-5,7-difluoro-1H-indazole (74 mg, 0.19 mmol) and triethylamine (40 μL, 0.28 mmol) in CH₂Cl₂ (1 mL) at 0° C. The mixture stirred at 0° C. to rt over 1 h and diluted with CH₂Cl₂ (10 mL). The organic layer was washed with water (10 mL), dried (Na₂SO₄), filtered, concentrated, and then purified by silica gel chromatography (0-12% EtOAc in CH₂Cl₂) to give 6-(benzyloxy)-5,7-difluoro-1-(4-(1-(methylsulfonyl)azetidin-3-yl)phenyl)-1H-indazole (65 mg, 73%) as a white solid. ¹H NMR (400 MHz, DMSO-d₆): δ 8.40 (s, 1H), 7.66 (d, J=9.5 Hz, 1H), 7.64-7.56 (m, 4H), 7.47-7.43 (m, 2H), 7.42-7.34 (m, 3H), 5.22 (s, 2H), 4.30-4.23 (m, 2H), 4.05-3.98 (m, 3H), 3.12 (s, 3H); LCMS: 470.1 [M+H]⁺.

### Step 4: 5,7-Difluoro-1-(4-(1-(methylsulfonyl)azetidin-3-yl)phenyl)-1H-indazol-6-ol

Palladium on carbon (10 wt. %) was added to a mixture of 6-(benzyloxy)-5,7-difluoro-1-(4-(1-(methylsulfonyl)azetidin-3-yl)phenyl)-1H-indazole (62 mg, 0.13 mmol), CH₃OH (1 mL), and EtOAc (3 mL) at rt under N₂. The mixture was degassed with 3 vacuum/H₂ cycles, stirred at rt for 5 h, and then filtered. The filter cake was washed with CH₃OH (3 mL), and the filtrate was concentrated to give 5,7-difluoro-1-(4-(1-(methylsulfonyl)azetidin-3-yl)phenyl)-1H-indazol-6-ol (50 mg, 100%) as an off-white foam. ¹H NMR (400 MHz, DMSO-d₆): δ 10.58 (s, 1H), 8.30 (s, 1H), 7.64-7.59 (m, 2H), 7.58-7.52 (m, 3H), 4.31-4.21 (m, 2H), 4.04-3.95 (m, 3H), 3.11 (s, 3H); LCMS: 379.9 [M+H]⁺.

The Compound below was synthesized from Intermediate 14.9 and 4,4,5,5-tetramethyl-2-(3-1,2,5,6-tetrahydropyridyl)-1,3,2-dioxaborolane hydrochloride following the procedures described for Compound 28 (Step 1) and Compound 30 (Steps 3-4).

| Cmpd | Structure | Name | [M + H]⁺ |
|---|---|---|---|
| 30.1 | 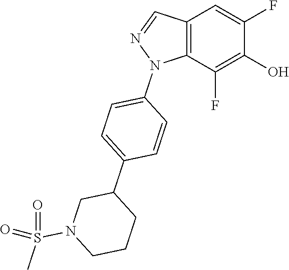 [SMILES: CS(=O)(=O)N1CCCC(c2ccc(-n3ncc4cc(F)c(O)c(F)c43)cc2)C1] | 5,7-Difluoro-1-(4-(1- (methylsulfonyl)piperidin-3- yl)phenyl)-1H-indazol-6-ol | 408.1 |

Alternate conditions: Step 1: 3 h; Step 3: 21 h; Step 4: Palladium on carbon (5 wt. %), THF, H₂, rt, 40 h.

### Compound 31

### 5,7-Difluoro-1-(4-((1-(methylsulfonyl)piperidin-4-yl)oxy)phenyl)-1H-indazol-6-ol

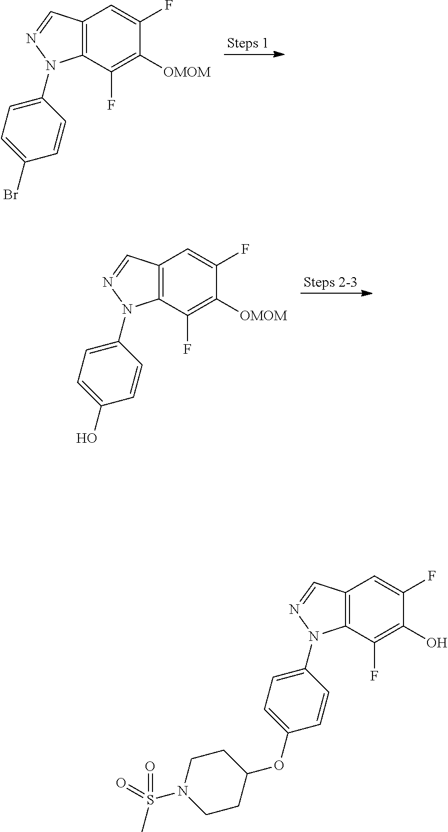

### Step 1: 4-(5,7-Difluoro-6-(methoxymethoxy)-1H-indazol-1-yl)phenol

A mixture of Intermediate 14.8 (217 mg, 0.59 mmol), tris(dibenzylideneacetone)dipalladium(0) (54 mg, 0.06 mmol), tBuXPhos (50 mg, 0.12 mmol), cesium carbonate (768 mg, 2.36 mmol), dioxane (3.5 mL), and water (0.7 mL) was degassed with 3 vacuum/N₂ cycles. The reaction was stirred at 90° C. for 1.5 h, allowed to cool to rt, and then diluted with 20 mL EtOAc and 20 mL water. The layers were separated. The EtOAc layer was washed with 20 mL brine, dried (Na₂SO₄), filtered, concentrated, and then purified by silica gel chromatography (10-30% EtOAc in hexanes) to give 4-(5,7-difluoro-6-(methoxymethoxy)-1H-indazol-1-yl)phenol (124 mg, 69%) as a yellow solid. LCMS: 306.9 [M+H]⁺.

### Step 2: 5,7-Difluoro-6-(methoxymethoxy)-1-(4-((1-(methylsulfonyl)piperidin-4-yl)oxy)phenyl)-1H-indazole

A solution of di-tert-butyl azodicarboxylate (137 mg, 0.59 mmol) in 0.5 mL CH₂Cl₂ was added to a solution of 4-[5,7-difluoro-6-(methoxymethoxy)-1H-indazolyl]phenol (122 mg, 0.40 mmol), triphenylphosphine (156 mg, 0.59 mmol), and 1-(methylsulfonyl)piperidin-4-ol (87 mg, 0.49 mmol) in 1.5 mL CH₂Cl₂ at 0° C. under N₂. The reaction was stirred at rt for 1.5 h and then purified by silica gel chromatography (20-60% EtOAc in hexanes) to give 5,7-difluoro-6-(methoxymethoxy)-1-(4-((1-(methylsulfonyl)piperidin-4-yl)oxy)phenyl)-1H-indazole (130.6 mg, 67%) as a white foam. LCMS: 468.0 [M+H]⁺.

### Step 3: 5,7-Difluoro-1-(4-((1-(methylsulfonyl)piperidin-4-yl)oxy)phenyl)-1H-indazol-6-ol

Trifluoroacetic acid (0.4 mL, 5.22 mmol) was added to a solution of 4-{4-[5,7-difluoro-6-(methoxymethoxy)(1H-indazolyl)]phenoxy}-1-(methylsulfonyl)piperidine (50 mg 0.11 mmol) in CH₂Cl₂ (2 mL). The reaction was stirred at rt for 1 h, concentrated, and then diluted with 20 mL EtOAc and 20 mL saturated NaHCO₃. The layers were separated. The EtOAc layer was washed with 20 mL saturated NaHCO₃, washed with 20 mL brine, dried (Na₂SO₄), filtered, concentrated, and then purified by silica gel chromatography (30-60% EtOAc in hexanes) to give 5,7-difluoro-1-(4-((1-(methylsulfonyl)piperidin-4-yl)oxy)phenyl)-1H-indazol-6-ol (28 mg, 61%) as a white foam. ¹H NMR (400 MHz, DMSO-d₆): δ 10.50 (s, 1H), 8.24 (s, 1H), 7.51 (br d, J=9.0 Hz, 3H), 7.13 (d, J=8.4 Hz, 2H), 4.65 (br d, J=3.2 Hz, 1H), 3.43-3.30 (m, 2H), 3.22-3.08 (m, 2H), 2.92 (s, 3H), 2.11-2.00 (m, 2H), 1.86-1.73 (m, 2H); LCMS: 424.1 [M+H]⁺.

### Compound 32

### 4-(4-(5,7-Difluoro-6-hydroxy-1H-indazol-1-yl)phenyl)-2,6-dimethylthiomorpholine 1,1-dioxide

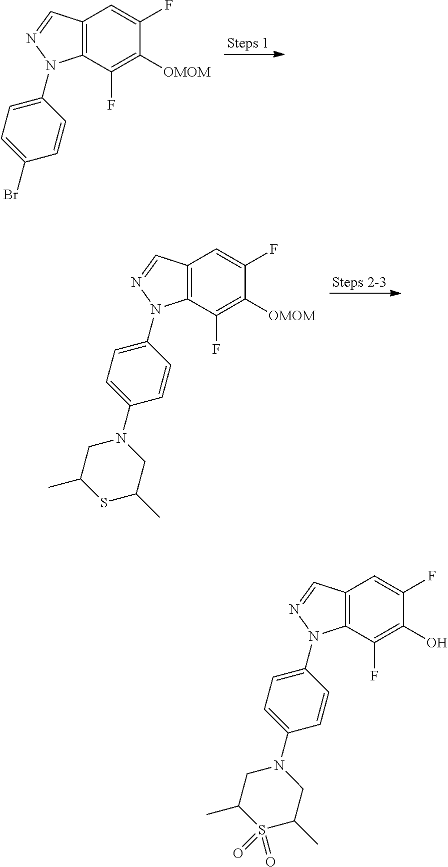

### Step 1: 4-(4-(5,7-Difluoro-6-(methoxymethoxy)-1H-indazol-1-yl)phenyl)-2,6-dimethylthiomorpholine

A mixture of Intermediate 14.8 (303 mg, 0.82 mmol), sodium tert-butoxide* (316 mg, 3.28 mmol), 2,6-dimethylthiomorpholine (225 mg, 1.71 mmol), 2-dicyclohexylphosphino-2′,6′-diisopropoxy-1,1′-biphenyl (39 mg, 0.08 mmol), tris(dibenzylideneacetone)dipalladium(0) (38 mg, 0.04 mmol), and toluene (4 mL) was degassed with 3 vacuum/N₂ cycles, stirred at 100° C. for 1 h, allowed to cool to rt, and then diluted with 20 mL EtOAc and 20 mL saturated NaHCO₃. The layers were separated. The organic layer was washed with 20 mL brine, dried (Na₂SO₄), filtered, concentrated, and then purified by silica gel chromatography (0-20% EtOAc in hexanes) to give 4-(4-(5,7-difluoro-6-(methoxymethoxy)-1H-indazol-1-yl)phenyl)-2,6-dimethylthiomorpholine (286 mg, 83%) as a yellow oil. ¹H NMR (400 MHz, DMSO-d₆): δ 8.33 (s, 1H), 7.63 (d, J=9.8 Hz, 1H), 7.46-7.38 (m, 2H), 7.09-7.01 (m, 2H), 5.19 (s, 2H), 4.19-4.10 (m, 0.8H), 3.63 (dd, J=2.2, 13.0 Hz, 1.3H), 3.49 (s, 3H), 3.34-3.28 (m, 1.4H), 3.20-3.12 (m, 1.3H), 3.11-3.03 (m, 0.8H), 2.79-2.69 (m, 0.8H), 1.29 (d, J=6.8 Hz, 4H), 1.13 (d, J=6.7 Hz, 2H); LCMS: 420.1 [M+H]⁺.

* Sodium tert-butoxide was dried under high vacuum by heating with a heat gun for a few minutes and then allowing to cool prior to weighing.

### Step 2: 4-(4-(5,7-Difluoro-6-(methoxymethoxy)-1H-indazol-1-yl)phenyl)-2,6-dimethylthiomorpholine 1,1-dioxide

A solution of 4-(4-(5,7-difluoro-6-(methoxymethoxy)-1H-indazol-1-yl)phenyl)-2,6-dimethylthiomorpholine (105 mg, 0.25 mmol) in CH₂Cl₂ (1.5 mL) was cooled in an ice/water bath. 3-Chloroperbenzoic acid (108 mg, 0.63 mmol) was added. The reaction was allowed to warm to rt, stirred for 16 h, and then diluted with 20 mL EtOAc and 20 mL saturated NaHCO₃. The layers were separated. The organic layer was washed with 20 mL saturated NaHCO₃, washed with 20 mL brine, dried (Na₂SO₄), filtered, concentrated, and then purified by silica gel chromatography (20-45% EtOAc in hexanes) to give 4-(4-(5,7-difluoro-6-(methoxymethoxy)-1H-indazol-1-yl)phenyl)-2,6-dimethylthiomorpholine 1,1-dioxide (41 mg, 36%) as an off-white foam. LCMS: 452.1 [M+H]⁺.

### Step 3: 4-(4-(5,7-Difluoro-6-hydroxy-1H-indazol-1-yl)phenyl)-2,6-dimethylthiomorpholine 1,1-dioxide

Trifluoroacetic acid (0.2 mL) was added to a solution of 4-(4-(5,7-difluoro-6-(methoxymethoxy)-1H-indazol-1-yl)phenyl)-2,6-dimethylthiomorpholine 1,1-dioxide (41 mg, 0.09 mmol) in CH₂Cl₂ (1 mL) at rt. The reaction was stirred for 30 min, concentrated, dried under high vacuum, and then purified by reverse-phase HPLC (72.4-82.4% CH₃CN in water with 0.1% TFA). The product fractions were combined and concentrated. The mixture was diluted with 20 mL EtOAc and 20 mL saturated NaHCO₃. The layers were separated. The organic layer was washed with 20 mL saturated NaHCO₃, washed with 20 mL brine, dried (Na₂SO₄), filtered, concentrated, and then dried under high vacuum to give 4-(4-(5,7-difluoro-6-hydroxy-1H-indazol-1-yl)phenyl)-2,6-dimethylthiomorpholine 1,1-dioxide (25 mg, 68%) as a yellow solid. ¹H NMR (400 MHz, DMSO-d₆): δ 10.47 (s, 1H), 8.22 (d, J=2.2 Hz, 1H), 7.50 (dd, J=1.0, 9.8 Hz, 1H), 7.41 (dd, J=2.7, 8.9 Hz, 2H), 7.14 (d, J=8.9 Hz, 2H), 3.80 (dd, J=2.1, 14.0 Hz, 2H), 3.58 (br dd, J=6.9, 14.0 Hz, 2H), 3.39 (dt, J=3.2, 7.0 Hz, 2H), 1.31 (d, J=7.0 Hz, 6H); LCMS: 408.1 [M+H]⁺.

The Compounds below were synthesized from Intermediate 14.8 and the appropriate amine following the procedures described for Compound 32.

| Cmpd | Structure | Name | [M+H]+ |
|---|---|---|---|
| 32.1 | 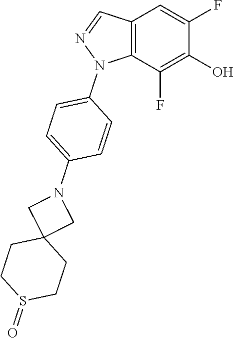 [SMILES: O=S1CCC2(CC1)CN(c1ccc(-n3ncc4cc(F)c(O)c(F)c43)cc1)C2] | 2-(4-(5,7-Difluoro-6-hydroxy-1H- indazol-1-yl)phenyl)-7-thia-2- azaspiro[3.5]nonane 7-oxide | 404.1 |
| 32.2 | 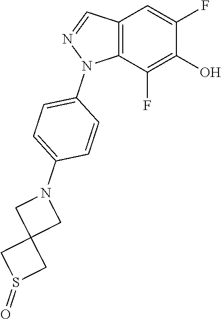 [SMILES: O=S1CC2(CN(c3ccc(-n4ncc5cc(F)c(O)c(F)c54)cc3)C2)C1] | 6-(4-(5,7-Difluoro-6-hydroxy-1H- indazol-1-yl)phenyl)-2-thia-6- azaspiro[3.3]heptane 2-oxide | 375.9 |
| 32.3 | 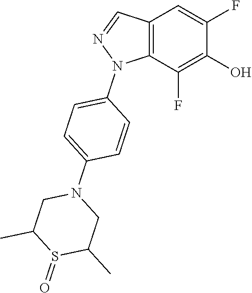 [SMILES: CC1CN(c2ccc(-n3ncc4cc(F)c(O)c(F)c43)cc2)CC(C)S1=O] | 4-(4-(5,7-Difluoro-6-hydroxy-1H- indazol-1-yl)phenyl)-2,6- dimethylthiomorpholine 1-oxide | 392.0 |
| 32.4¹ | 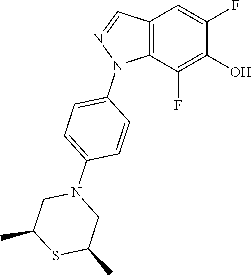 [SMILES: C[C@@H]1CN(c2ccc(-n3ncc4cc(F)c(O)c(F)c43)cc2)C[C@H](C)S1] | 1-(4-(cis-2,6- Dimethylthiomorpholino)phenyl)- 5,7-difluoro-1H-indazol-6-ol | 376.1 |
| 32.5¹ | 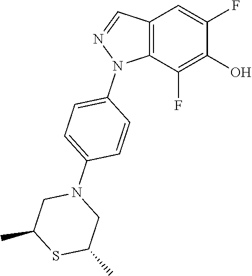 [SMILES: C[C@H]1CN(c2ccc(-n3ncc4cc(F)c(O)c(F)c43)cc2)C[C@H](C)S1] | 1-(4-(trans-2,6- Dimethylthiomorpholino)phenyl)- 5,7-difluoro-1H-indazol-6-ol | 376.0 |

Alternate conditions: Step 1: 1-4 h. Step 2: for sulfoxides: 15 min-4 h; m-CPBA added at-78° C. or 0° C.
¹No oxidation step; cis/trans isomers separated by reverse-phase HPLC.

### Compound 33

### 1-(4-(4,4-Dimethylpiperidin-1-yl)phenyl)-5,7-difluoro-6-hydroxyindolin-2-one

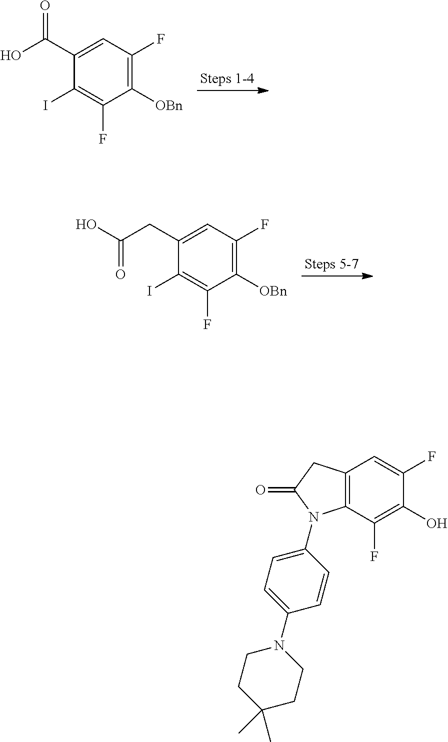 [SMILES: O=C(O)c1cc(F)c(OCc2ccccc2)c(F)c1I ; O=C(O)Cc1cc(F)c(OCc2ccccc2)c(F)c1I ; CC1(C)CCN(c2ccc(N3C(=O)Cc4cc(F)c(O)c(F)c43)cc2)CC1]

### Step 1: 4-(Benzyloxy)-3,5-difluoro-2-iodobenzoyl chloride

Thionyl chloride (27.5 g, 231 mmol) was added to a solution of Intermediate 4, Step 2 (45 g, 115 mmol), DMF (843 mg, 11.5 mmol), and toluene (450 mL) under N₂. The mixture was stirred at 85° C. for 2 h, allowed to cool to rt, and then concentrated to give 4-(benzyloxy)-3,5-difluoro-2-iodobenzoyl chloride (58 g) as a yellow oil.

### Step 2: 1-(4-(Benzyloxy)-3,5-difluoro-2-iodophenyl)-2-diazoethanone

(Trimethylsilyl)diazomethane solution (2 M in THF, 213 mL, 426 mmol) was added to a solution of 4-(benzyloxy)-3,5-difluoro-2-iodobenzoyl chloride (58 g, 142 mmol), THF (400 mL), and CH₃CN (400 mL) at 0° C. under N₂. The mixture was allowed to warm to rt, stirred overnight, concentrated, and then purified by silica gel chromatography (petroleum ether/EtOAc=9/1) to give 1-(4-(benzyloxy)-3,5-difluoro-2-iodophenyl)-2-diazoethanone (38 g, 88% over 3 steps) as a yellow solid. ¹H NMR (400 MHz, DMSO-d₆): δ 7.51-7.27 (m, 6H), 6.51 (s, 1H), 5.23 (s, 2H); LCMS: 414.8 [M+H]⁺.

### Step 3: tert-Butyl 2-(4-(benzyloxy)-3,5-difluoro-2-iodophenyl)acetate

(Benzoyloxy)silver (6.27 g, 27.4 mmol) was added to a solution of 1-(4-(benzyloxy)-3,5-difluoro-2-iodophenyl)-2-diazoethanone (38 g, 91 mmol), dioxane (400 mL), and t-BuOH (400 mL). The mixture was stirred at rt overnight, poured into water (1.2 L), and then extracted with EtOAc (3×300 mL). The combined organic layers were washed with brine (400 ml), dried over Na₂SO₄, filtered, concentrated, and then purified by silica gel chromatography (EtOAc/petroleum ether=0/1) to give tert-butyl 2-(4-(benzyloxy)-3,5-difluoro-2-iodophenyl)acetate (30.2 g, 72%) as a yellow oil. ¹H NMR (400 MHz, DMSO-d₆): δ 7.46-7.33 (m, 5H), 7.31-7.23 (m, 1H), 5.16 (s, 2H), 3.73 (s, 2H), 1.40 (s, 9H).

### Step 4: 2-(4-(Benzyloxy)-3,5-difluoro-2-iodophenyl)acetic Acid

A mixture of tert-butyl 2-(4-(benzyloxy)-3,5-difluoro-2-iodophenyl)acetate (28 g, 61 mmol) and HCl in dioxane (4 N, 300 mL) was stirred at rt overnight and then concentrated to give 2-(4-(benzyloxy)-3,5-difluoro-2-iodophenyl)acetic acid (24 g) as a yellow solid.

### Step 5: 2-(4-(Benzyloxy)-2-((4-(4,4-dimethylpiperidin-1-yl)phenyl)amino)-3,5-difluorophenyl)acetic Acid

Cu powder (189 mg, 2.97 mmol) and CuI (566 mg, 2.97 mmol) were added to a mixture of 2-(4-(benzyloxy)-3,5-difluoro-2-iodophenyl)acetic acid (12 g, 30 mmol), Intermediate 32 (6.07 g, 29.7 mmol), K₂CO₃ (12.3 g, 89.1 mmol), and dry DMF (240 mL) under N₂. The reaction mixture was stirred at 100° C. overnight, allowed to cool to rt, poured into water (500 mL), and then extracted with EtOAc (4×100 mL). The combined organic layers were washed with brine (100 mL), dried over Na₂SO₄, filtered, concentrated, and then purified by silica gel chromatography (CH₂Cl₂/CH₃OH=0/1). The material was purified further by reverse prep-HPLC [water(0.5% HCl)—CH₃OH] to give 2-(4-(benzyloxy)-2-((4-(4,4-dimethylpiperidin-1-yl)phenyl)amino)-3,5-difluorophenyl)acetic acid (1.36 g) as a black brown solid. ¹H NMR (400 MHz, DMSO-d₆): δ 12.23 (s, 1H), 11.09 (s, 1H), 7.55-7.26 (m, 5H), 7.21-6.92 (m, 2H), 6.74 (s, 1H), 6.54-6.21 (m, 2H), 5.15 (s, 2H), 3.52 (s, 2H), 3.28-2.73 (m, 4H), 1.72-1.32 (m, 4H), 0.97 (s, 6H); LCMS: 481.3 [M+H]⁺.

### Step 6: 6-(Benzyloxy)-1-(4-(4,4-dimethylpiperidin-1-yl)phenyl)-5,7-difluoroindolin-2-one

Phosphorus(V) oxychloride (0.08 mL, 0.91 mmol) was added to a solution of 2-(4-(benzyloxy)-2-((4-(4,4-dimethylpiperidin-1-yl)phenyl)amino)-3,5-difluorophenyl)acetic acid (290 mg, 0.603 mmol), pyridine (0.10 mL, 1.2 mmol), and THF (5 mL) at 0° C. under N₂. The mixture was stirred at rt for 2 h, poured into ice-cold sat. aq. NaHCO₃ (20 mL), and then extracted with EtOAc (3×10 mL). The combined organic layers were washed with brine (10 mL), dried over Na₂SO₄, filtered, concentrated, and then purified by silica gel chromatography (petroleum ether/EtOAc=80/20). The material was further purified by prep-HPLC [water(0.05% HCl)—CH₃CN] to give 6-(benzyloxy)-1-(4-(4,4-dimethylpiperidin-1-yl)phenyl)-5,7-difluoroindolin-2-one (30 mg, 11%) as a yellow solid. ¹H NMR (400 MHz, DMSO-d₆): δ 7.42-7.32 (m, 5H), 7.20-7.09 (m, 3H), 7.03-6.91 (m, 2H), 5.05 (s, 2H), 3.72 (s, 2H), 3.25-3.16 (m, 4H), 1.49-1.39 (m, 4H), 0.97 (s, 6H); LCMS: 463.3 [M+H]⁺.

### Step 7: 1-(4-(4,4-Dimethylpiperidin-1-yl)phenyl)-5,7-difluoro-6-hydroxyindolin-2-one

6-(Benzyloxy)-1-(4-(4,4-dimethylpiperidin-1-yl)phenyl)-5,7-difluoroindolin-2-one (30 mg, 0.065 mmol) was added to a mixture of Pd/C (10%, 10 mg) in CH₃OH (5 mL) under N₂. The suspension was degassed under vacuum and purged with H₂ several times. The mixture was stirred under H₂ (15 psi) at rt for 2 h, and then filtered. The Celite pad was washed with CH₃OH (30 mL). The filtrate was concentrated and then purified by silica gel chromatography (petroleum ether/EtOAc=3/1) to give 20 mg crude, which was combined with another 14 mg crude. The combined 34 mg crude was purified by silica gel chromatography (petroleum ether/EtOAc=3/1) to give 1-(4-(4,4-dimethylpiperidin-1-yl)phenyl)-5,7-difluoro-6-hydroxyindolin-2-one (25 mg, 60%) as a yellow solid. ¹H NMR (400 MHz, DMSO-d₆): δ 10.00 (s, 1H), 7.16 (d, 2H), 7.05 (d, 1H), 6.97 (d, 2H), 3.65 (s, 2H), 3.25-3.14 (m, 4H), 1.50-1.40 (m, 4H), 0.96 (s, 6H); LCMS: 373.2 [M+H]⁺.

### Compound 34

### 1-(4-(4,4-Dimethylpiperidin-1-yl)phenyl)-5,7-difluoro-6-hydroxy-3,3-dimethylindolin-2-one

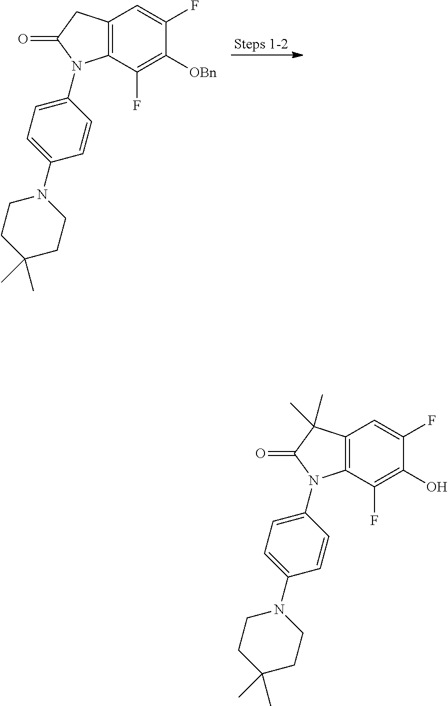 [SMILES: CC1(C)CCN(c2ccc(N3C(=O)Cc4cc(F)c(OCc5ccccc5)c(F)c43)cc2)CC1 ; CC1(C)CCN(c2ccc(N3C(=O)C(C)(C)c4cc(F)c(O)c(F)c43)cc2)CC1]

### Step 1: 6-(Benzyloxy)-1-(4-(4,4-dimethylpiperidin-1-yl)phenyl)-5,7-difluoro-3,3-dimethylindolin-2-one

Lithium diisopropylamide (2 M in THF, 1.62 mL, 3.24 mmol) was added dropwise to a solution of Compound 33, Step 6 (600 mg, 1.30 mmol), HMPA (1.16 g, 6.49 mmol), and THF (20 mL) under N₂ at −78° C. The mixture was stirred for 1 h. Iodomethane (0.2 mL, 3.24 mmol) was added. The mixture was stirred for 1 h, allowed to warm to rt, stirred for 2 h, poured into sat. aq. NH₄Cl (40 mL), and then extracted with EtOAc (3×20 mL). The combined organic layers were washed with brine (20 mL), dried over Na₂SO₄, filtered, concentrated, and then purified by silica gel chromatography (petroleum ether/EtOAc=9/1) to give 6-(benzyloxy)-1-(4-(4,4-dimethylpiperidin-1-yl)phenyl)-5,7-difluoro-3,3-dimethylindolin-2-one (400 mg, 62%) as a white solid. ¹H NMR (400 MHz, DMSO-d₆): δ 7.42-7.30 (m, 6H), 7.18 (d, 2H), 6.98 (d, 2H), 5.05 (s, 2H), 3.28-3.15 (m, 4H), 1.49-1.42 (m, 4H), 1.36 (s, 6H), 0.97 (s, 6H); LCMS: 491.2 [M+H]⁺.

### Step 2: 1-(4-(4,4-Dimethylpiperidin-1-yl)phenyl)-5,7-difluoro-6-hydroxy-3,3-dimethylindolin-2-one

6-(Benzyloxy)-1-(4-(4,4-dimethylpiperidin-1-yl)phenyl)-5,7-difluoro-3,3-dimethylindolin-2-one (390 mg, 0.795 mmol) was added to a mixture of Pd/C (100 mg, 10% purity) in CH₃OH (20 mL) under N₂. The suspension was degassed under vacuum and purged with H₂ several times. The mixture was stirred under H₂ (15 psi) at rt for 2 h and then filtered. The Celite pad was washed with CH₃OH (200 mL). The filtrate was concentrated and then purified by silica gel chromatography (petroleum ether/EtOAc=9/1) to give 1-(4-(4,4-dimethylpiperidin-1-yl)phenyl)-5,7-difluoro-6-hydroxy-3,3-dimethylindolin-2-one (260 mg, 82%) as a white solid. ¹H NMR (400 MHz, DMSO-d₆): δ 10.02 (s, 1H), 7.22 (d, 1H), 7.16 (d, 2H), 6.98 (d, 2H), 3.28-3.15 (m, 4H), 1.53-1.41 (m, 4H), 1.32 (s, 6H), 0.99 (s, 6H); LCMS: 401.2 [M+H]⁺.

### Compound 35

### 1-(4-(4,4-Dimethylpiperidin-1-yl)phenyl)-5,7-difluoro-3,6-dihydroxyindolin-2-one hydrochloride

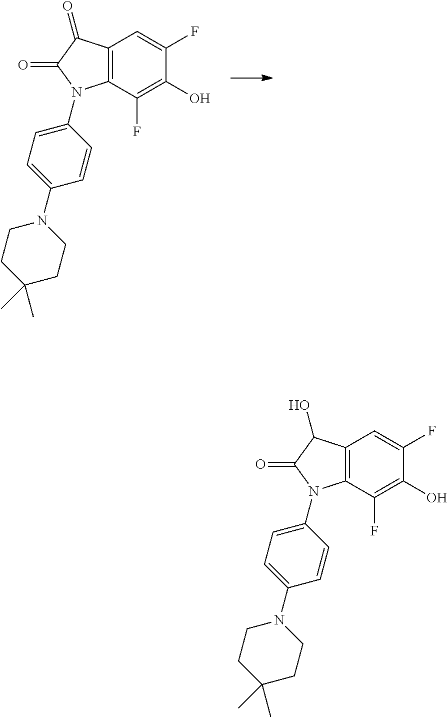 [SMILES: CC1(C)CCN(c2ccc(N3C(=O)C(=O)c4cc(F)c(O)c(F)c43)cc2)CC1 ; CC1(C)CCN(c2ccc(N3C(=O)C(O)c4cc(F)c(O)c(F)c43)cc2)CC1]

Triethylsilane (0.42 mL, 2.59 mmol) and BF₃·Et₂O (0.34 mL, 2.59 mmol) were added to a mixture of Compound 7.2 (50 mg, 0.129 mmol) and CH₂Cl₂ (4 mL) at −78° C. under N₂. The mixture was stirred at rt overnight, poured into sat. aq. NaHCO₃ (10 mL), and then extracted with CH₂Cl₂ (3×10 mL). The combined organic layers were dried over Na₂SO₄, filtered, and concentrated. The residue was combined with a 5 mg batch and purified by prep-HPLC [water(0.05% HCl)—CH₃CN] to give 1-(4-(4,4-dimethylpiperidin-1-yl)phenyl)-5,7-difluoro-3,6-dihydroxyindolin-2-one hydrochloride (19.8 mg, 33%) as a white solid. ¹HNMR (400 MHz, DMSO-d₆): δ 10.46-10.13 (m, 2H), 7.66-7.19 (m, 5H), 7.12 (d, 1H), 5.05-5.00 (m, 1H), 3.42-3.29 (m, 4H), 1.72-1.48 (m, 4H), 1.03 (s, 6H); LCMS: 389.2 [M+H]⁺.

### Compound 36

### 1-(4-(4,4-Dimethylpiperidin-1-yl)phenyl)-5,7-difluoro-6-hydroxy-1H-indazol-3(2H)-one

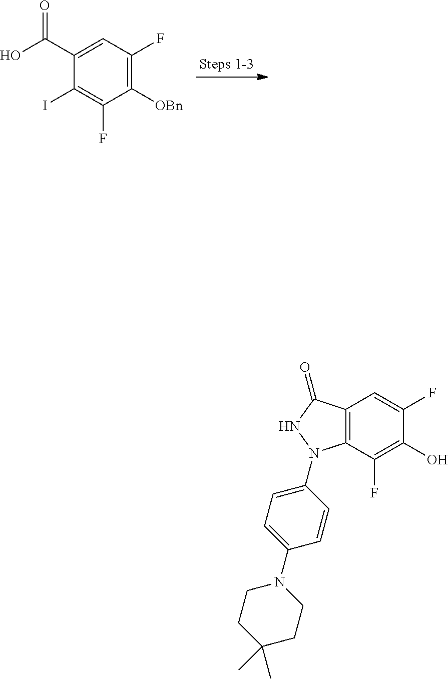 [SMILES: O=C(O)c1cc(F)c(OCc2ccccc2)c(F)c1I ; CC1(C)CCN(c2ccc(-n3[nH]c(=O)c4cc(F)c(O)c(F)c43)cc2)CC1]

### Step 1: 4-(Benzyloxy)-N′-(4-(4,4-dimethylpiperidin-1-yl)phenyl)-3,5-difluoro-2-iodobenzohydrazide

Propylphosphonic anhydride solution (50% in EtOAc, 19.0 mL, 32.0 mmol) was added to a mixture of Intermediate 4, Step 2 (5 g, 13 mmol), Intermediate 28.1 (4.92 g, 19.2 mmol, HCl), Et₃N (5.19 g, 51.3 mmol), and CH₂Cl₂ (80 mL). The mixture was stirred at rt for 2 h, poured into water (100 mL), and then extracted with CH₂Cl₂ (3×50 mL). The combined organic layers were washed with brine (50 mL), dried over Na₂SO₄, filtered, concentrated, and then purified by silica gel chromatography (EtOAc/CH₃OH=9/1). The material was purified further by prep-HPLC [water(0.05% HCl)—CH₃CN] to give 4-(benzyloxy)-N-(4-(4,4-dimethylpiperidin-1-yl)phenyl)-3,5-difluoro-2-iodobenzohydrazide (650 mg, 8%) as a yellow solid. ¹H NMR (400 MHz, DMSO-d₆): δ 10.13 (d, 1H), 7.55 (d, 1H), 7.50-7.28 (m, 6H), 6.87-6.76 (m, 4H), 5.23 (s, 2H), 3.01-2.90 (m, 4H), 1.51-1.34 (m, 4H), 0.94 (s, 6H); LCMS: 592.2 [M+H]⁺.

### Step 2: 6-(Benzyloxy)-1-(4-(4,4-dimethylpiperidin-1-yl)phenyl)-5,7-difluoro-1H-indazol-3(2H)-one

Copper(I) iodide (20.9 mg, 0.110 mmol) was added to a mixture of 4-(benzyloxy)-N-(4-(4,4-dimethylpiperidin-1-yl)phenyl)-3,5-difluoro-2-iodobenzohydrazide (650 mg, 1.10 mmol), K₂CO₃ (304 mg, 2.20 mmol), L-proline (25.3 mg, 0.220 mmol), and DMSO (8 mL) under N₂. The mixture was stirred at 70° C. overnight, allowed to cool to rt, poured into water (50 mL), and then extracted with EtOAc (3×20 mL). The combined organic layers were dried over Na₂SO₄, filtered, concentrated, and then purified by prep-HPLC [water(0.04% HCl)—CH₃CN] to give 6-(benzyloxy)-1-(4-(4,4-dimethylpiperidin-1-yl)phenyl)-5,7-difluoro-1H-indazol-3(2H)-one (100 mg, 20%) as a gray solid. ¹H NMR (400 MHz, DMSO-d₆): δ 12.09-10.80 (m, 1H), 7.68-7.21 (m, 10H), 5.20 (s, 2H), 3.52-3.18 (m, 4H), 1.81-1.42 (m, 4H), 1.03 (s, 6H); LCMS: 464.3 [M+H]⁺.

### Step 3: 1-(4-(4,4-Dimethylpiperidin-1-yl)phenyl)-5,7-difluoro-6-hydroxy-1H-indazol-3(2H)-one

6-(Benzyloxy)-1-(4-(4,4-dimethylpiperidin-1-yl)phenyl)-5,7-difluoro-1H-indazol-3(2H)-one (30 mg, 0.065 mmol) was added to a mixture of Pd/C (10%, 20 mg) in CH₃OH (5 mL) under N₂. The suspension was degassed under vacuum and purged with H₂ several times. The mixture was stirred under H₂ (15 psi) at rt for 2 h and then filtered. The Celite pad was washed with CH₃OH (50 mL). The filtrate was combined and concentrated. The crude material was combined with another batch of the same scale and purified by prep-HPLC [water(0.04% HCl)—CH₃CN] to give 1-(4-(4,4-dimethylpiperidin-1-yl)phenyl)-5,7-difluoro-6-hydroxy-1H-indazol-3(2H)-one (13.6 mg, 28%) as a white solid. ¹H NMR (400 MHz, DMSO-d₆): δ 10.54 (s, 1H), 7.99-6.97 (m, 5H), 3.52-3.24 (m, 4H), 1.79-1.38 (m, 4H), 1.04 (s, 6H); LCMS: 374.1 [M+H]⁺.

### Compound 37

### 1-(4-(4,4-Dimethylpiperidin-1-yl)phenyl)-5,7-difluoro-6-hydroxy-2-methyl-1H-indazol-3(2H)-one

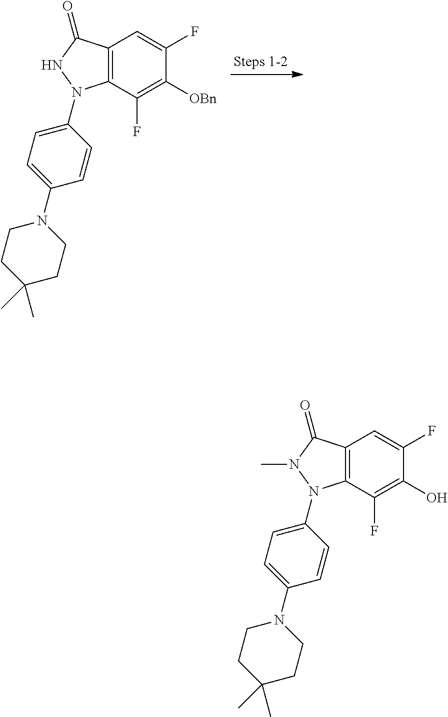 [SMILES: CC1(C)CCN(c2ccc(-n3[nH]c(=O)c4cc(F)c(OCc5ccccc5)c(F)c43)cc2)CC1 ; Cn1c(=O)c2cc(F)c(O)c(F)c2n1-c1ccc(N2CCC(C)(C)CC2)cc1]

### Step 1: 6-(Benzyloxy)-1-(4-(4,4-dimethylpiperidin-1-yl)phenyl)-5,7-difluoro-2-methyl-1H-indazol-3(2H)-one

Iodomethane (32.2 mg, 0.227 mmol) was added to a mixture of K₂CO₃ (41.7 mg, 0.302 mmol), Compound 36, Step 2 (70 mg, 0.15 mmol), and DMF (2 mL) under N₂. The mixture was stirred at rt overnight, poured into water (10 mL), and then extracted with MTBE (3×10 mL). The combined organic layers were dried over Na₂SO₄, filtered, concentrated, and then purified by chromatography on silica gel (petroleum ether/EtOAc=9/1 to 0/1) to give 6-(benzyloxy)-1-(4-(4,4-dimethylpiperidin-1-yl)phenyl)-5,7-difluoro-2-methyl-1H-indazol-3(2H)-one (20 mg, 28%) and 6-(benzyloxy)-1-(4-(4,4-dimethylpiperidin-1-yl)phenyl)-5,7-difluoro-3-methoxy-1H-indazole (50 mg, 69%) as a yellow solid. 6-(benzyloxy)-1-(4-(4,4-dimethylpiperidin-1-yl)phenyl)-5,7-difluoro-2-methyl-1H-indazol-3(2H)-one: ¹HNMR (400 MHz, DMSO-d₆): δ 7.54 (d, 1H), 7.38-7.33 (m, 5H), 7.13-7.08 (m, 2H), 7.01-6.95 (m, 2H), 5.19 (s, 2H), 3.26-3.21 (m, 4H), 3.07 (s, 3H), 1.47-1.41 (m, 4H), 0.97 (s, 6H); LCMS: 478.2 [M+H]⁺. 6-(benzyloxy)-1-(4-(4,4-dimethylpiperidin-1-yl)phenyl)-5,7-difluoro-3-methoxy-1H-indazole: ¹H NMR (400 MHz, DMSO-d₆): δ 7.47-7.27 (m, 8H), 7.06-6.97 (m, 2H), 5.19 (s, 2H), 4.02 (s, 3H), 3.27-3.18 (m, 4H), 1.50-1.42 (m, 4H), 0.98 (s, 6H); LCMS: 478.2 [M+H]⁺.

### Step 2: 1-(4-(4,4-Dimethylpiperidin-1-yl)phenyl)-5,7-difluoro-6-hydroxy-2-methyl-1H-indazol-3(2H)-one

6-(Benzyloxy)-1-(4-(4,4-dimethylpiperidin-1-yl)phenyl)-5,7-difluoro-2-methyl-1H-indazol-3(2H)-one (20 mg, 0.042 mmol) was added to a mixture of Pd/C (10%, 20 mg) in CH₃OH (5 mL) under N₂. The suspension was degassed under vacuum and purged with H₂ several times. The mixture was stirred under H₂ (15 psi) at rt for 2 h and then filtered. The Celite pad was washed with CH₃OH (50 mL). The filtrate was concentrated and then purified by prep-HPLC [water (0.04% HCl)—CH₃CN] to give 1-(4-(4,4-dimethylpiperidin-1-yl)phenyl)-5,7-difluoro-6-hydroxy-2-methyl-1H-indazol-3(2H)-one (4.6 mg, 28%) as a yellow solid. ¹H NMR (400 MHz, MeOD-d₄): δ 7.89 (d, 2H), 7.64 (d, 2H), 7.41 (dd, 1H), 3.78-3.60 (m, 4H), 3.22 (s, 3H), 1.98-1.80 (m, 4H), 1.19 (s, 6H); LCMS: 388.1 [M+H]⁺.

The Compound below was synthesized from 6-(benzyloxy)-1-(4-(4,4-dimethylpiperidin-1-yl)phenyl)-5,7-difluoro-3-methoxy-1H-indazole (isolated from Compound 37, Step 1) following the procedure described for Compound 37, Step 2.

| Cmpd | Structure | Name | [M + H]⁺ |
|---|---|---|---|
| 37.1 | 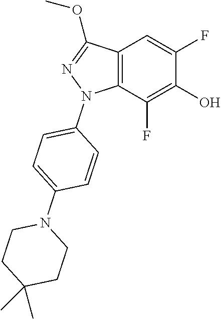 [SMILES: COc1nn(-c2ccc(N3CCC(C)(C)CC3)cc2)c2c(F)c(O)c(F)cc12] | 1-(4-(4,4-Dimethylpiperidin-1- yl)phenyl)-5,7-difluoro-3- methoxy-1H-indazol-6-ol | 388.3 |

### Compound 38

### 7-Bromo-3-(4-(4,4-dimethylpiperidin-1-yl)phenyl)-4,6-difluoro-5-hydroxybenzo[d]oxazol-2(3H)-one

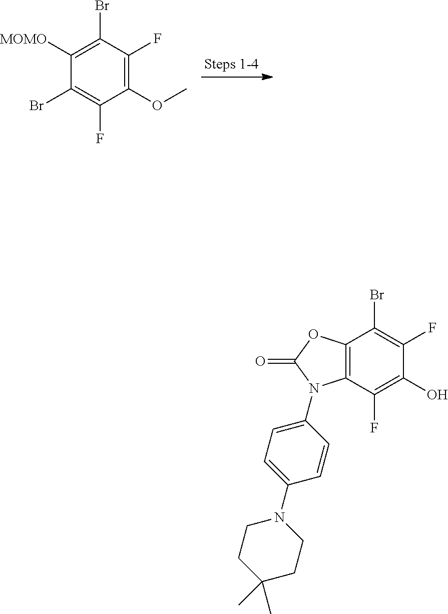

### Step 1: 3-Bromo-N-(4-(4,4-dimethylpiperidin-1-yl)phenyl)-4,6-difluoro-5-methoxy-2-(methoxymethoxy)aniline

XantPhos (480 mg, 0.829 mmol) and Pd₂(dba)₃ (380 mg, 0.414 mmol) were added to a solution of Intermediate 38 (1.5 g, 4.14 mmol), Intermediate 32 (847 mg, 4.14 mmol), Cs₂CO₃ (4.05 g, 12.4 mmol), and dioxane (20 mL) under N₂. The mixture was degassed under vacuum and purged with N₂ several times, allowed to warm to 100° C., stirred overnight, allowed to cool to rt, and then filtered through Celite. The filter cake was washed with EtOAc (30 mL). The filtrate was concentrated and then purified silica gel chromatography (petroleum ether/EtOAc=95/5) to give 3-bromo-N-(4-(4,4-dimethylpiperidin-1-yl)phenyl)-4,6-difluoro-5-methoxy-2-(methoxymethoxy)aniline (1.3 g) as a yellow solid. ¹H NMR (400 MHz, DMSO-d₆): δ 7.32 (s, 1H), 6.80 (d, 2H), 6.58 (d, 2H), 4.93 (s, 2H), 3.91 (s, 3H), 3.40 (s, 3H), 3.00-2.90 (m, 4H), 1.46-1.37 (m, 4H), 0.93 (s, 6H); MS: 485.2 [M+H]⁺.

### Step 2: 2-Bromo-6-((4-(4,4-dimethylpiperidin-1-yl)phenyl)amino)-3,5-difluoro-4-methoxyphenol

A mixture of 3-bromo-N-(4-(4,4-dimethylpiperidin-1-yl)phenyl)-4,6-difluoro-5-methoxy-2-(methoxymethoxy)aniline (1.3 g, 2.41 mmol), CH₂Cl₂ (12 mL), and TFA (3 mL) was stirred at rt for 2 h, concentrated, diluted with sat. NaHCO₃ (20 mL), and then extracted with CH₂Cl₂ (3×10 mL). The combined organic layers were washed with brine (10 mL), dried over Na₂SO₄, filtered, concentrated, and then purified by silica gel chromatography (petroleum ether/EtOAc=9/1) to give 2-bromo-6-((4-(4,4-dimethylpiperidin-1-yl)phenyl)amino)-3,5-difluoro-4-methoxyphenol (750 mg) as a yellow solid. ¹H NMR (400 MHz, DMSO-d₆): δ 9.88 (s, 1H), 7.02-6.73 (m, 3H), 6.67-6.44 (m, 2H), 3.83 (s, 3H), 3.05-2.86 (m, 4H), 1.51-1.38 (m, 4H), 0.94 (s, 6H); LCMS: 441.2 [M+H]⁺.

### Step 3: 7-Bromo-3-(4-(4,4-dimethylpiperidin-1-yl)phenyl)-4,6-difluoro-5-methoxybenzo[d]oxazol-2(3H)-one

A mixture of 2-bromo-6-((4-(4,4-dimethylpiperidin-1-yl)phenyl)amino)-3,5-difluoro-4-methoxyphenol (705 mg, 1.60 mmol), CDI (518 mg, 3.20 mmol), and THF (8 mL) was stirred at 60° C. overnight, allowed to cool to rt, concentrated, and then purified by silica gel chromatography (petroleum ether/EtOAc=95/5) to give 7-bromo-3-(4-(4,4-dimethylpiperidin-1-yl)phenyl)-4,6-difluoro-5-methoxybenzo[d]oxazol-2(3H)-one (300 mg, 16% over 3 steps) as a white solid. ¹H NMR (400 MHz, DMSO-d₆): δ 7.38 (d, 2H), 7.04 (d, 2H), 3.88 (s, 3H), 3.29-3.20 (m, 4H), 1.50-1.39 (m, 4H), 0.97 (s, 6H); LCMS: 467.1 [M+H]⁺.

### Step 4: 7-Bromo-3-(4-(4,4-dimethylpiperidin-1-yl)phenyl)-4,6-difluoro-5-hydroxybenzo[d]oxazol-2(3H)-one

Sodium iodide (111 mg, 0.738 mmol) was added to a solution of 7-bromo-3-(4-(4,4-dimethylpiperidin-1-yl)phenyl)-4,6-difluoro-5-methoxybenzo[d]oxazol-2(3H)-one (230 mg, 0.492 mmol) in HBr (300% w/w in water, 10 mL). The mixture was stirred at 100° C. overnight, allowed to cool to rt, adjusted to pH˜7 with sat. NaHCO₃, and then extracted with EtOAc (3×10 mL). The combined organic layers were washed with brine (10 mL), dried over Na₂SO₄, filtered, concentrated, and then purified by silica gel chromatography (petroleum ether/EtOAc=9/1 to 4/1). The material was purified further by prep-TLC (petroleum ether/EtOAc=5/1) to give 7-bromo-3-(4-(4,4-dimethylpiperidin-1-yl)phenyl)-4,6-difluoro-5-hydroxybenzo[d]oxazol-2(3H)-one (55 mg, 25%) as a white solid. ¹H NMR (400 MHz, DMSO-d₆): δ 10.53 (s, 1H), 7.51-7.37 (m, 2H), 7.31-7.03 (m, 2H), 3.34-3.26 (m, 4H), 1.58-1.44 (m, 4H), 0.99 (s, 6H); LCMS: 453.0 [M+H]⁺.

### Compound 39

### 3-(4-(4,4-Dimethylpiperidin-1-yl)phenyl)-4,6-difluoro-5-hydroxybenzo[d]oxazol-2(3H)-one

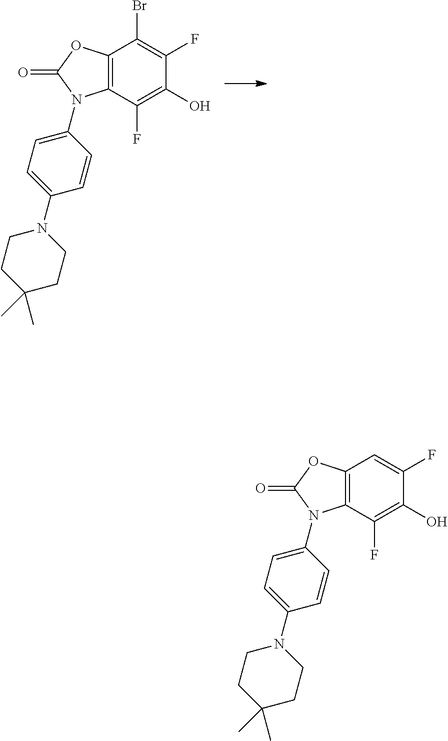 [SMILES: CC1(C)CCN(c2ccc(-n3c(=O)oc4c(Br)c(F)c(O)c(F)c43)cc2)CC1 ; CC1(C)CCN(c2ccc(-n3c(=O)oc4cc(F)c(O)c(F)c43)cc2)CC1]

Compound 38 (50 mg, 0.110 mmol) was added to a mixture of Pd/C (10%, 50 mg) in CH₃OH (5 mL) under N₂. The suspension was degassed under vacuum and purged with H₂ several times, stirred under H₂ (15 psi) at 30° C. overnight, and then filtered. The Celite pad was washed with CH₃OH (50 mL). The filtrate was concentrated and combined with another batch (10 mg scale). The combined mixture was purified by prep-HPLC [water(0.04% HCl)—CH₃CN] to give 3-(4-(4,4-dimethylpiperidin-1-yl)phenyl)-4,6-difluoro-5-hydroxybenzo[d]oxazol-2(3H)-one (17.1 mg, 34%) as a white solid. ¹H NMR (400 MHz, DMSO-d₆): δ 10.06 (s, 1H), 7.42-7.29 (m, 3H), 7.09-6.95 (m, 2H), 3.27-3.19 (m, 4H), 1.49-1.40 (m, 4H), 0.97 (s, 6H); LCMS: 375.1 [M+H]⁺.

### Compound 40

### 1-(4-(7-Oxa-2-azaspiro[3.5]nonan-2-yl)phenyl)-4-chloro-5-fluoro-1H-benzo[d][1,2,3]triazol-6-ol

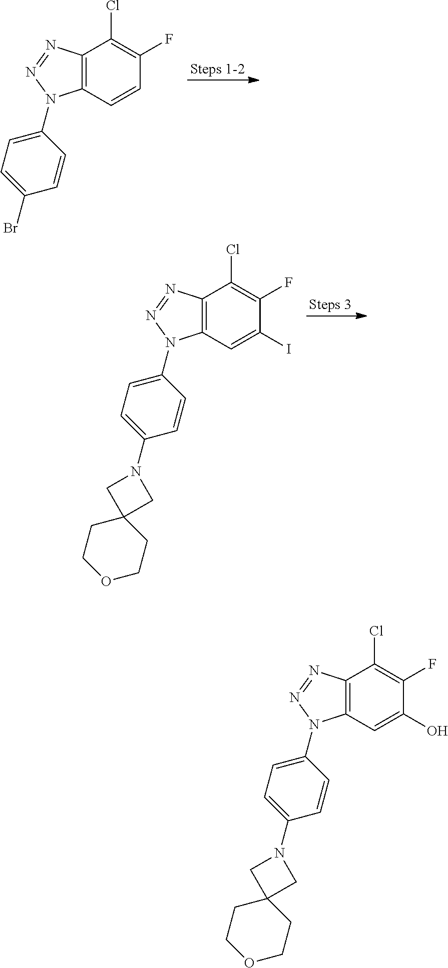 [SMILES: Fc1ccc2c(nnn2-c2ccc(Br)cc2)c1Cl ; Fc1c(I)cc2c(nnn2-c2ccc(N3CC4(CCOCC4)C3)cc2)c1Cl ; Oc1cc2c(nnn2-c2ccc(N3CC4(CCOCC4)C3)cc2)c(Cl)c1F]

### Step 1: 2-(4-(4-Chloro-5-fluoro-1H-benzo[d][1,2,3]triazol-1-yl)phenyl)-7-oxa-2-azaspiro[3.5]nonane

RuPhos (85.7 mg, 0.184 mmol) and Pd₂(dba)₃ (84 mg, 0.092 mmol) were added to a mixture of Intermediate 40 (300 mg, 0.919 mmol), 7-oxa-2-azaspiro[3.5]nonane (180 mg, 1.10 mmol, HCl), Cs₂CO₃ (1.20 g, 3.67 mmol), and dioxane (10 mL) under N₂. The mixture was degassed and purged with N₂ for 3 times, warmed to 80° C., stirred for 3 h, allowed to cool to rt, poured into water (20 mL), and then extracted with EtOAc (3×20 mL). The combined organic layers were washed with brine (20 mL), dried over Na₂SO₄, filtered, concentrated, and then purified by silica gel chromatography (petroleum ether/EtOAc=3/1) to give 2-(4-(4-chloro-5-fluoro-1H-benzo[d][1,2,3]triazol-1-yl)phenyl)-7-oxa-2-azaspiro[3.5]nonane (185 mg, 54%) as a black/brown solid. ¹H NMR (400 MHz, DMSO-d₆): δ 7.79-7.74 (m, 1H), 7.72-7.66 (m, 1H), 7.62-7.56 (m, 2H), 6.68-6.60 (m, 2H), 3.74-3.68 (m, 4H), 3.60-3.54 (m, 4H), 1.80-1.73 (m, 4H); LCMS: 373.1 [M+H]⁺.

### Step 2: 2-(4-(4-Chloro-5-fluoro-6-iodo-1H-benzo[d][1,2,3]triazol-1-yl)phenyl)-7-oxa-2-azaspiro[3.5]nonane

Lithium diisopropylamide (2 M in THF, 0.56 mL, 1.12 mmol) was added to a solution of 2-(4-(4-chloro-5-fluoro-1H-benzo[d][1,2,3]triazol-1-yl)phenyl)-7-oxa-2-azaspiro[3.5]nonane (350 mg, 0.939 mmol) in THF (4 mL) at −78° C. under N₂. The mixture was stirred for 1 h. Iodine (357 mg, 1.41 mmol) in THF (4 mL) was added dropwise to the mixture at −78° C. The mixture was allowed to warm to rt slowly, stirred overnight, quenched with sat. aq. Na₂SO₃ (15 mL), and then extracted with EtOAc (3×10 mL). The combined organic layers were washed with brine (10 mL), dried over Na₂SO₄, filtered, concentrated, and then purified by silica gel chromatography (petroleum ether/EtOAc=1/1) to give 2-(4-(4-chloro-5-fluoro-6-iodo-1H-benzo[d][1,2,3]triazol-1-yl)phenyl)-7-oxa-2-azaspiro[3.5]nonane (265 mg, 57%) as a yellow solid. ¹HNMR (400 MHz, DMSO-d₆): δ 8.20 (d, 1H), 7.59 (d, 2H), 6.63 (d, 2H), 3.76-3.68 (m, 4H), 3.62-3.53 (m, 4H), 1.82-1.74 (m, 4H); LCMS: 498.9 [M+H]⁺.

### Step 3: 1-(4-(7-Oxa-2-azaspiro[3.5]nonan-2-yl)phenyl)-4-chloro-5-fluoro-1H-benzo[d][1,2,3]triazol-6-ol

2-Di-t-butylphosphino-2′,4′,6′-tri-1-propyl-1,1′-biphenyl (21.5 mg, 0.051 mmol) and Pd₂(dba)₃ (11.6 mg, 0.013 mmol) were added to a mixture of 2-(4-(4-chloro-5-fluoro-6-iodo-1H-benzo[d][1,2,3]triazol-1-yl)phenyl)-7-oxa-2-azaspiro[3.5]nonane (63 mg, 0.126 mmol), KOH (28.4 mg, 0.505 mmol), dioxane (2 mL) and H₂O (2 mL) under N₂. The mixture was degassed and purged with N₂ for 3 times, warmed to 100° C., stirred for 3 h, and then allowed to cool to rt. The mixture was combined with three other reaction mixtures of the same scale, poured into water (20 mL), adjusted to pH˜2 with HCl (1 N), and then extracted with EtOAc (3×20 mL). The combined organic layers were washed with brine (20 mL), dried over Na₂SO₄, filtered, concentrated, and then purified by prep-HPLC [water(0.2% formic acid)-CH₃CN]. The material was purified further by silica gel chromatography (petroleum ether/EtOAc=1/1) to give 1-(4-(7-oxa-2-azaspiro[3.5]nonan-2-yl)phenyl)-4-chloro-5-fluoro-1H-benzo[d][1,2,3]triazol-6-ol (14.5 mg, 70%) as a white solid. ¹H NMR (400 MHz, DMSO-d₆): δ 11.20 (s, 1H), 7.54-7.47 (m, 2H), 7.01 (d, 1H), 6.68-6.59 (m, 2H), 3.75-3.66 (m, 4H), 3.61-3.54 (m, 4H), 1.82-1.74 (m, 4H); LCMS: 389.0 [M+H]⁺.

The Compounds below were synthesized from the Intermediate 40 or 40.1 and the appropriate amine following the procedures described for Compound 40.

| Cmpd | Structure | Name | [M + H]⁺ |
|---|---|---|---|
| 40.1 | 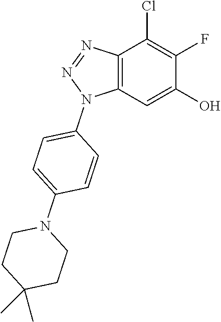 [SMILES: CC1(C)CCN(c2ccc(-n3nnc4c(Cl)c(F)c(O)cc43)cc2)CC1] | 4-Chloro-1-(4-(4,4- dimethylpiperidin-1-yl)phenyl)-5- fluoro-1H-benzo[d][1,2,3]triazol- 6-ol | 375.0 |
| 40.2 | 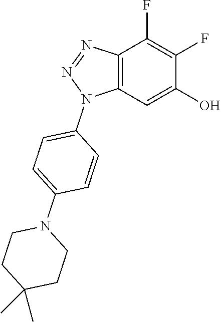 [SMILES: CC1(C)CCN(c2ccc(-n3nnc4c(F)c(F)c(O)cc43)cc2)CC1] | 1-(4-(4,4-Dimethylpiperidin-1- yl)phenyl)-4,5-difluoro-1H- benzo[d][1,2,3]triazol-6-ol | 359.1 |
| 40.3 | 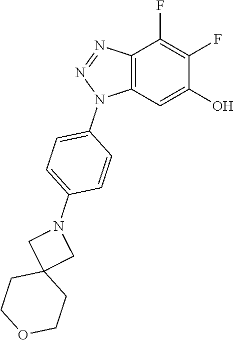 [SMILES: Oc1cc2c(nnn2-c2ccc(N3CC4(CCOCC4)C3)cc2)c(F)c1F] | 1-(4-(7-oxa-2-azaspiro[3.5]nonan- 2-yl)phenyl)-4,5-difluoro-1H- benzo[d][1,2,3]triazol-6-ol | 373.1 |

### Compound 41

### 1-(4-(7-Oxa-2-azaspiro[3.5]nonan-2-yl)phenyl)-5-fluoro-4-(trifluoromethyl)-1H-benzo[d][1,2,3]triazol-6-ol

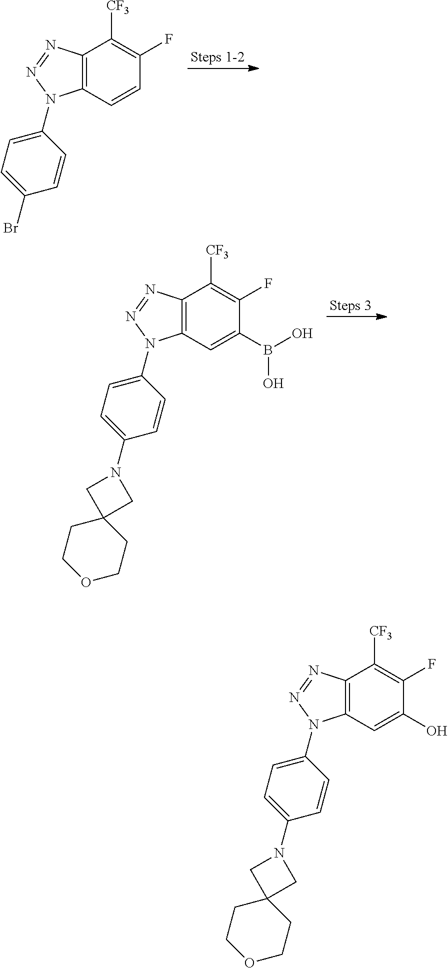 [SMILES: Fc1ccc2c(nnn2-c2ccc(Br)cc2)c1C(F)(F)F ; OB(O)c1cc2c(nnn2-c2ccc(N3CC4(CCOCC4)C3)cc2)c(C(F)(F)F)c1F ; Oc1cc2c(nnn2-c2ccc(N3CC4(CCOCC4)C3)cc2)c(C(F)(F)F)c1F]

### Step 1: 2-(4-(5-Fluoro-4-(trifluoromethyl)-1H-benzo[d][1,2,3]triazol-1-yl)phenyl)-7-oxa-2-azaspiro[3.5]nonane

RuPhos (117 mg, 0.250 mmol) and Pd₂(dba)₃ (114 mg, 0.125 mmol) were added to a mixture of Intermediate 40.2 (450 mg, 1.25 mmol), 7-oxa-2-azaspiro[3.5]nonane hydrochloride (245 mg, 1.50 mmol), Cs₂CO₃ (1.63 g, 5.00 mmol), and dioxane (10 mL) under N₂. The mixture was degassed and purged with N₂ for 3 times, warmed to 80° C., stirred overnight, allowed to cool to rt, poured into water (20 mL), and then extracted with EtOAc (3×10 mL). The combined organic layers were washed with brine (10 mL), dried over Na₂SO₄, filtered, concentrated, and then purified by silica gel chromatography (petroleum ether/EtOAc=3/1) to give 2-(4-(5-fluoro-4-(trifluoromethyl)-1H-benzo[d][1,2,3]triazol-1-yl)phenyl)-7-oxa-2-azaspiro[3.5]nonane (320 mg, 63%) as a yellow solid. ¹HNMR (400 MHz, DMSO-d₆): δ 8.11 (dd, 1H), 7.74 (t, 1H), 7.60 (d, 2H), 6.65 (d, 2H), 3.76-3.68 (m, 4H), 3.63-3.52 (m, 4H), 1.82-1.74 (m, 4H); LCMS: 407.1 [M+H]⁺.

### Step 2: (1-(4-(7-Oxa-2-azaspiro[3.5]nonan-2-yl)phenyl)-5-fluoro-4-(trifluoromethyl)-1H-benzo[d][1,2,3]triazol-6-yl)boronic Acid

Lithium diisopropylamide (2 M in THF, 0.54 mL, 1.08 mmol) was added dropwise to a solution of 2-(4-(5-fluoro-4-(trifluoromethyl)-1H-benzo[d][1,2,3]triazol-1-yl)phenyl)-7-oxa-2-azaspiro[3.5]nonane (290 mg, 0.714 mmol) in THF (5 mL) at −78° C. under N₂. The mixture was stirred for 1 h. Trimethyl borate (148 mg, 1.43 mmol) in THF (5 mL) was added dropwise to the mixture at −78° C. The reaction mixture was warmed to rt slowly, stirred overnight, quenched with sat. aq. NH₄Cl (20 mL), and then extracted with EtOAc (3×10). The combined organic layers were washed with brine (10 mL), dried over Na₂SO₄, filtered, and then concentrated to give (1-(4-(7-oxa-2-azaspiro[3.5]nonan-2-yl)phenyl)-5-fluoro-4-(trifluoromethyl)-1H-benzo[d][1,2,3]triazol-6-yl)boronic acid (600 mg) as a yellow solid. LCMS: 451.1 [M+H]⁺.

### Step 3: 1-(4-(7-Oxa-2-azaspiro[3.5]nonan-2-yl)phenyl)-5-fluoro-4-(trifluoromethyl)-1H-benzo[d][1,2,3]triazol-6-ol

Hydrogen peroxide (0.64 mL, 6.66 mmol) was added dropwise to a solution of (1-(4-(7-oxa-2-azaspiro[3.5]nonan-2-yl)phenyl)-5-fluoro-4-(trifluoromethyl)-1H-benzo[d][1,2,3]triazol-6-yl)boronic acid (600 mg, 1.33 mmol) in THF (10 mL) at rt. The reaction mixture was stirred overnight, quenched with sat. aq. Na₂SO₃ (20 mL), and then extracted with EtOAc (3×10 mL). The combined organic layers were dried over Na₂SO₄, filtered, concentrated, and then purified by prep-HPLC [water(0.2% formic acid)-CH₃CN] to give 1-(4-(7-oxa-2-azaspiro[3.5]nonan-2-yl)phenyl)-5-fluoro-4-(trifluoromethyl)-1H-benzo[d][1,2,3]triazol-6-ol (168 mg, 56% over 2 steps) as a yellow solid. ¹H NMR (400 MHz, DMSO-d₆): δ 11.40 (s, 1H), 7.56-7.48 (m, 2H), 7.30 (d, 1H), 6.67-6.61 (m, 2H), 3.76-3.68 (m, 4H), 3.61-3.52 (m, 4H), 1.85-1.69 (m, 4H); LCMS: 423.0 [M+H]⁺.

The Compound below was synthesized from Intermediate 40.2 and 4,4-dimethylpiperidine hydrochloride following the procedures described for Compound 41.

| Cmpd | Structure | Name | [M + H]⁺ |
|---|---|---|---|
| 41.1 | 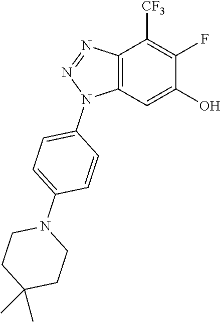 [SMILES: CC1(C)CCN(c2ccc(-n3nnc4c(C(F)(F)F)c(F)c(O)cc43)cc2)CC1] | 1-(4-(4,4-Dimethylpiperidin-1- yl)phenyl)-5-fluoro-4- (trifluoromethyl)-1H- benzo[d][1,2,3]triazol-6-ol | 409.0 |

### Compound 42

### 1-(2-(4,4-Dimethylpiperidin-1-yl)pyrimidin-5-yl)-5,7-difluoro-1H-benzo[d][1,2,3]triazol-6-ol

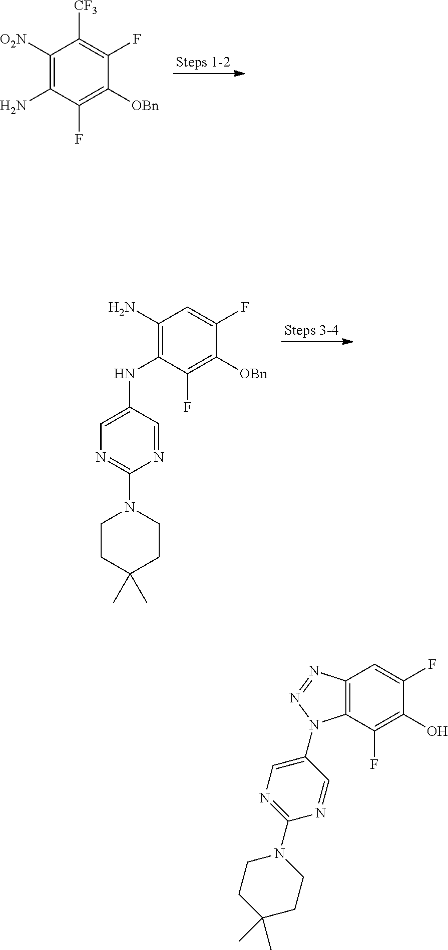 [SMILES: Nc1c(F)c(OCc2ccccc2)c(F)c(C(F)(F)F)c1[N+](=O)[O-] ; CC1(C)CCN(c2ncc(Nc3c(N)cc(F)c(OCc4ccccc4)c3F)cn2)CC1 ; CC1(C)CCN(c2ncc(-n3nnc4cc(F)c(O)c(F)c43)cn2)CC1]

### Step 1: N-(3-(benzyloxy)-2,4-difluoro-6-nitrophenyl)-2-(4,4-dimethylpiperidin-1-yl)pyrimidin-5-amine

Copper(I) iodide (17 mg, 0.089 mmol) was added to a solution of 3-(benzyloxy)-2,4-difluoro-6-nitroaniline (251 mg, 0.892 mmol), Intermediate 30.1 (311 mg, 0.981 mmol), (1S,2S)—N1,N2-dimethylcyclohexane-1,2-diamine (63.5 mg, 0.446 mmol), and K₃PO₄ (568 mg, 2.68 mmol) in dioxane (6 mL) under N₂. The mixture was stirred at 100° C. for 12 h, allowed to cool to rt, diluted with H₂O (40 mL), and then extracted with EtOAc (3×30 mL). The combined organic layers were washed with brine (40 mL), dried over Na₂SO₄, filtered, concentrated, and then purified by silica gel chromatography (petroleum ether/ethyl acetate=27/1) to give N-(3-(benzyloxy)-2,4-difluoro-6-nitrophenyl)-2-(4,4-dimethylpiperidin-1-yl)pyrimidin-5-amine (490 mg, 59%) as a red solid. LCMS: 470.2 [M+H]⁺.

### Step 2: 5-(Benzyloxy)-N′-(2-(4,4-dimethylpiperidin-1-yl)pyrimidin-5-yl)-4,6-difluorobenzene-1,2-diamine

Ammonium chloride (335 mg, 6.26 mmol) and then Fe powder (350 mg, 6.26 mmol) were added to a solution of N-(3-benzyloxy-2,4-difluoro-6-nitro-phenyl)-2-(4,4-dimethyl-1-piperidyl)pyrimidin-5-amine (490 mg, 1.04 mmol) in H₂O (2 mL) and THF (8 mL). The mixture was stirred at 70° C. for 1.5 h and then filtered. The filtrate was concentrated and purified by silica gel chromatography (petroleum ether/ethyl acetate=8/1) to give 5-(benzyloxy)-N¹-(2-(4,4-dimethylpiperidin-1-yl)pyrimidin-5-yl)-4,6-difluorobenzene-1,2-diamine (402 mg, 79%) as a red oil. LCMS: 440.2 [M+H]⁺.

### Step 3: 6-(Benzyloxy)-1-(2-(4,4-dimethylpiperidin-1-yl)pyrimidin-5-yl)-5,7-difluoro-1H-benzo[d][1,2,3]triazole

tert-Butyl nitrite (164 mg, 1.59 mmol) was added to a solution of 5-(benzyloxy)-N¹-(2-(4,4-dimethylpiperidin-1-yl)pyrimidin-5-yl)-4,6-difluorobenzene-1,2-diamine (199 mg, 0.46 mmol) in CH₃CN (10 mL). The mixture was stirred at 25° C. for 1 h, concentrated, and then purified by prep-TLC (petroleum ether/ethyl acetate=4/1) to give 6-(benzyloxy)-1-(2-(4,4-dimethylpiperidin-1-yl)pyrimidin-5-yl)-5,7-difluoro-1H-benzo[d][1,2,3]triazole (78 mg, 38%) as a white solid. LCMS: 451.2 [M+H]⁺.

### Step 4: 1-(2-(4,4-Dimethylpiperidin-1-yl)pyrimidin-5-yl)-5,7-difluoro-1H-benzo[d][1,2,3]triazol-6-ol

A mixture of 6-(benzyloxy)-1-(2-(4,4-dimethylpiperidin-1-yl)pyrimidin-5-yl)-5,7-difluoro-1H-benzo[d][1,2,3]triazole (68 mg, 0.151 mmol) in TFA (3 mL) was stirred at 50° C. for 2 h. The reaction mixture was allowed to cool to 25° C., concentrated, and then purified by prep-HPLC [water(0.2% formic acid)-CH₃CN] to give 1-(2-(4,4-dimethylpiperidin-1-yl)pyrimidin-5-yl)-5,7-difluoro-1H-benzo[d][1,2,3]triazol-6-ol (25.1 mg, 46%) as a white solid. ¹HNMR (400 MHz, DMSO-d₆): δ 11.10 (s, 1H), 8.71 (d, 2H), 8.00-7.88 (m, 1H), 3.92-3.80 (m, 4H), 1.44-1.34 (m, 4H), 1.01 (s, 6H); LCMS: 361.1 [M+H]⁺.

The Compound below was synthesized using Intermediate 30.2 following the procedures described for Compound 42.

| Cmpd | Structure | Name | [M + H]⁺ |
|---|---|---|---|
| 42.1 | 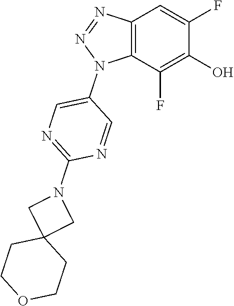 [SMILES: Oc1c(F)cc2nnn(-c3cnc(N4CC5(CCOCC5)C4)nc3)c2c1F] | 1-(2-(7-Oxa-2-azaspiro[3.5]nonan- 2-yl)pyrimidin-5-yl)-5,7-difluoro- 1H-benzo[d][1,2,3]triazol-6-ol | 375.2 |

### Example A-1: Parenteral Pharmaceutical Composition

To prepare a parenteral pharmaceutical composition suitable for administration by injection (subcutaneous, intravenous), 1-1000 mg of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s), and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 10-500 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound described herein, or a pharmaceutically acceptable salt thereof, is placed into size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl cellulose, propylene glycol, isopropyl myristate and purified alcohol USP. The resulting gel mixture is then incorporated into containers, such as tubes, which are suitable for topical administration.

### Example B-1: HSD17b13 NAD(P)H-Glo Biochemical Assay

### Materials

Recombinant human HSD17B13 enzyme. Substrate: estradiol (Sigma β-Estradiol E8875), 100 mM in DMSO. Cofactor: NAD+ Grade I free acid (Sigma 10127965001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 1% DMSO.

### Procedure

HSD17b13 enzyme was diluted in 1× assay buffer to the desired enzyme concentration based on the specific activity of the enzyme lot. 20 uL of diluted enzyme was added to each well along with 2.5 uL of 10× inhibitor solution. Assay plate was incubated at RT for 20 minutes, and then 2.5 uL of a 10× substrate/cofactor mix was added to each well for a final concentration of 50 uM estradiol and 1 mM NAD+. Assay plate was incubated at 37° C. for 3 hours. NAD(P)H-Glo™ Detection System reagents were prepared according to manufacturer's specifications, and 25 uL was added to each well. After incubating for 1 hour at RT, luminescence was measured.

Representative data for exemplary compounds disclosed herein is presented in Table 2.

TABLE 2

| | HSD17β13 |
|---|---|
| Cmpd | IC₅₀ (uM) |
| 1 | ++ |
| 2 | ++ |
| 2.1 | ++ |
| 2.2 | ++ |
| 2.3 | ++ |
| 2.4 | ++ |
| 2.5 | ++ |
| 2.6 | + |
| 2.7 | ++ |
| 2.8 | + |
| 2.9 | + |
| 3 | ++ |
| 4 | ++ |
| 4.1 | ++ |
| 4.2 | ++ |
| 4.3 | ++ |
| 5 | ++ |
| 5.1 | ++ |
| 5.2 | ++ |
| 5.3 | ++ |
| 5.4 | ++ |
| 6 | ++ |
| 6.1 | +++ |
| 6.2 | ++ |
| 7 | ++ |
| 7.1 | ++ |
| 7.2 | ++ |
| 7.3 | ++ |
| 7.4 | + |
| 8 | ++ |
| 9 | +++ |
| 10 | +++ |
| 11 | +++ |
| 12 | ++ |
| 13 | +++ |
| 13.1 | +++ |
| 13.2 | ++ |
| 13.3 | ++ |
| 13.4 | ++ |
| 13.5 | ++ |
| 13.6 | ++ |
| 13.7 | ++ |
| 13.8 | ++ |
| 13.9 | ++ |
| 13.10 | ++ |
| 13.11 | ++ |
| 13.12 | +++ |
| 13.13 | + |
| 13.14 | 40% at 30 uM |
| 13.15 | 40% at 30 uM |
| 13.16 | ++ |
| 13.17 | ++ |
| 13.18 | +++ |
| 13.19 | +++ |
| 13.20 | ++ |
| 13.21 | ++ |
| 13.22 | + |
| 13.23 | ++ |
| 13.24 | + |
| 13.25 | ++ |
| 13.26 | ++ |
| 13.27 | + |
| 13.28 | ++ |
| 13.29 | + |
| 13.30 | + |
| 13.31 | + |
| 13.32 | 40% at 100 uM |
| 13.33 | + |
| 13.34 | ++ |
| 13.35 | ++ |
| 13.36 | ++ |
| 13.37 | ++ |
| 13.38 | +++ |
| 13.39 | +++ |
| 13.40 | ++ |
| 14 | +++ |
| 14.1 | ++ |
| 14.2 | ++ |
| 14.3 | +++ |
| 14.4 | +++ |
| 15 | ++ |
| 15.1 | ++ |
| 15.2 | ++ |
| 15.3 | ++ |
| 15.4 | ++ |
| 16 | ++ |
| 17 | ++ |
| 18 | +++ |
| 18.1 | ++ |
| 18.2 | +++ |
| 18.3 | +++ |
| 18.4 | +++ |
| 19 | ++ |
| 19.1 | +++ |
| 20 | +++ |
| 20.1 | +++ |
| 20.2 | ++ |
| 20.3 | +++ |
| 20.4 | +++ |
| 20.5 | +++ |
| 20.6 | ++ |
| 20.7 | +++ |
| 20.8 | +++ |
| 20.9 | +++ |
| 20.10 | +++ |
| 20.11 | ++ |
| 20.12 | +++ |
| 20.13 | ++ |
| 20.14 | ++ |
| 20.15 | + |
| 20.16 | ++ |
| 20.17 | ++ |
| 20.18 | +++ |
| 20.19 | +++ |
| 20.20 | +++ |
| 20.21 | ++ |
| 20.22 | ++ |
| 20.23 | ++ |
| 21 | +++ |
| 21.1 | + |
| 21.2 | + |
| 21.3 | +++ |
| 21.4 | ++ |
| 21.5 | +++ |
| 21.6 | +++ |
| 21.7 | +++ |
| 21.8 | 35% at 10 uM |
| 21.9 | ++ |
| 21.10 | 35% at 10 uM |
| 21.11 | ++ |
| 21.12 | +++ |
| 21.13 | ++ |
| 22 | +++ |
| 22.1 | ++ |
| 22.2 | ++ |
| 22.3 | ++ |
| 22.4 | +++ |
| 22.5 | +++ |
| 23 | +++ |
| 23.1 | ++ |
| 23.2 | +++ |
| 23.3 | ++ |
| 23.4 | +++ |
| 23.5 | ++ |
| 23.6 | ++ |
| 23.7 | ++ |
| 23.8 | ++ |
| 23.9 | ++ |
| 23.10 | +++ |
| 23.11 | +++ |
| 23.12 | ++ |
| 23.13 | +++ |
| 23.14 | ++ |
| 23.15 | ++ |
| 23.16 | ++ |
| 23.17 | ++ |
| 23.18 | +++ |
| 23.19 | +++ |
| 23.20 | +++ |
| 23.21 | +++ |
| 23.22 | ++ |
| 23.23 | +++ |
| 23.24 | +++ |
| 23.25 | +++ |
| 23.26 | +++ |
| 23.27 | +++ |
| 23.28 | ++ |
| 24 | +++ |
| 24.1 | ++ |
| 24.2 | ++ |
| 24.3 | +++ |
| 24.4 | +++ |
| 24.5 | +++ |
| 24.6 | +++ |
| 24.7 | +++ |
| 24.8 | +++ |
| 24.9 | +++ |
| 24.10 | +++ |
| 24.11 | ++ |
| 24.12 | 35% at 10 uM |
| 25 | ++ |
| 25.1 | ++ |
| 25.2 | + |
| 25.3 | 35% at 10 uM |
| 25.4 | <20% at 10 uM |
| 25.5 | <20% at 10 uM |
| 25.6 | +++ |
| 26 | +++ |
| 26.1 | ++ |
| 26.2 | ++ |
| 26.3 | +++ |
| 26.4 | + |
| 26.5 | 35% at 10 uM |
| 26.6 | +++ |
| 26.7 | ++ |
| 26.8 | ++ |
| 26.9 | ++ |
| 26.10 | ++ |
| 26.11 | +++ |
| 26.12 | ++ |
| 26.13 | ++ |
| 26.14 | +++ |
| 26.15 | ++ |
| 26.16 | ++ |
| 26.17 | +++ |
| 26.18 | ++ |
| 26.19 | ++ |
| 26.20 | +++ |
| 26.21 | ++ |
| 26.22 | 45% at 10 uM |
| 26.23 | ++ |
| 26.24 | + |
| 26.25 | + |
| 26.26 | ++ |
| 26.27 | ++ |
| 26.28 | ++ |
| 26.29 | ++ |
| 27 | +++ |
| 27.1 | +++ |
| 27.2 | +++ |
| 27.3 | +++ |
| 27.4 | +++ |
| 27.5 | +++ |
| 27.6 | +++ |
| 27.7 | +++ |
| 27.8 | +++ |
| 27.9 | +++ |
| 27.10 | ++ |
| 28 | +++ |
| 29 | +++ |
| 30 | ++ |
| 30.1 | +++ |
| 31 | ++ |
| 32 | ++ |
| 32.1 | ++ |
| 32.2 | ++ |
| 32.3 | ++ |
| 32.4 | +++ |
| 32.5 | +++ |
| 33 | ++ |
| 34 | + |
| 35 | <20% at 10 uM |
| 36 | ++ |
| 37 | ++ |
| 37.1 | ++ |
| 38 | +++ |
| 39 | +++ |
| 40 | +++ |
| 40.1 | +++ |
| 40.2 | ++ |
| 40.3 | ++ |
| 41 | ++ |
| 41.1 | ++ |
| 42 | +++ |
| 42.1 | + |

where ‘+++’ means IC₅₀ ≤ 0.1 uM; where ‘++’ means 0.1 uM < IC₅₀ ≤ 1 uM; where ‘+’ means 1.0 uM < IC₅₀ ≤ 30 uM.

### Example B-2: HSD17b1 NAD(P)H-Glo Biochemical Assay

### Materials

Recombinant human HSD17B1 enzyme. Substrate: testosterone (Sigma T1500), 100 mM in DMSO. Cofactor: NADP disodium salt (Sigma 10128031001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 10% DMSO.

### Procedure

HSD17b1 enzyme was diluted in 1× assay buffer to the desired enzyme concentration based on the specific activity of the enzyme lot. 20 uL of diluted enzyme was added to each well along with 2.5 uL of the 10× inhibitor solution. Assay plate was incubated at RT for 20 minutes, and then 2.5 uL of a 10× substrate/cofactor mix was added to each well for a final concentration of 55 uM testosterone and 1 mM NADP. Assay plate was incubated at 37° C. for 1 hour. NAD(P)H-Glo™ Detection System reagents were prepared according to manufacturer's specifications, and 25 uL was added to each well. After incubating for 1 hour at RT, luminescence was measured.

### Example B-3: HSD17b2 NAD(P)H-Glo Biochemical Assay

### Materials and Setup

Recombinant human HSD17B2 enzyme. Substrate: estradiol (Sigma β-Estradiol E8875) 2 mM in DMSO. Cofactor: NAD+ Grade I free acid (Sigma 10127965001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 1% DMSO.

### Procedure

HSD17b2 enzyme was diluted in 1× assay buffer to the desired enzyme concentration based on the specific activity of the enzyme lot. 20 uL of diluted enzyme was added to each well along with 2.5 uL of 10× inhibitor solution. Assay plate was incubated at RT for 20 minutes, and then 2.5 uL of 10× substrate/cofactor mix was added to each well for a final assay concentration of 1 uM estradiol and 500 uM NAD+. Assay plate was incubated at RT for 1 hour. NAD(P)H-Glo™ Detection System reagents were prepared according to manufacturer's specifications and 25 uL was added to each well. After incubating for 1 hour at RT, luminescence was measured.

### Example B-4: In Vitro HSD17b13 Cell Based Assay

### Seeding

HEK293 cells were plated at 4,000,000 cells per T75 flask with EMEM (ATCC Cat #30-2003) and 10% FBS (Sigma Cat #F2442) and then incubated at 37° C. in 5% CO₂ for 18 hours.

### Transfection and Plate

After the 18 h incubation, media was replaced with 15 mL of fresh media: EMEM without Phenol Red (Quality Biological Cat #112-212-101), 10% CSS (Sigma Cat #F6765) and GlutaMax (Gibco Cat #35050-061). In a polypropylene tube, 20 ugpCMV6 HSD17B13 (Origene Cat #RC213132) was diluted in OptiMEM (Life Technologies, Cat #31985-062) to 2 mL. 60 uL of transfection reagent (X-tremeGENE HP Roche, Cat #06 366 236 001) was added, and the tube was vortexed and incubated at room temperature for 20 minutes. The transfection reagent/DNA mixture was added to the cells in the T75 flask, and the cells were incubated at 37° C. in 5% CO₂ for 18 hours. The next day, the cells were resuspended in EMEM media with 10% CSS and plated in a 96 well plate at 80,000 cells/well, 100 uL/well. Cells were incubated at 37° C. in 5% CO₂ for 18 hours.

### Test Compounds

Compounds were serially diluted in DMSO (1000× final concentration) and then further diluted in EMEM media with 10% CSS to a 20× final concentration. 10 uL of the 20× compound mix was added to each well of transfected cells, and the cells were incubated at 37° C. in 5% CO₂ for 30 minutes. 100 uL of EMEM media with 100 uM estradiol (Sigma cat #E8875) was added to each well, and the cells were incubated for 4 hours at 37° C. in 5% CO₂. The cell media was collected and examined for estradiol and estrone concentrations by LCMS.

### Example B-5: In Vitro HSD17b11 Cell Based Assay

### Seeding

HEK293 cells were plated at 4,000,000 cells per T75 flask with EMEM (ATCC Cat #30-2003) and 10% FBS (Sigma Cat #F2442) and then incubated at 37° C. in 5% CO₂ for 18 hours.

### Transfection and Plate

After the 18 h incubation, the media was replaced with 15 mL of fresh media: EMEM without Phenol Red (Quality Biological Cat #112-212-101), 10% CSS (Sigma Cat #F6765) and GlutaMax (Gibco Cat #35050-061). In a polypropylene tube, 20 ugpCMV6 HSD17B11 (Origene Cat #RC205941) was diluted in OptiMEM (Life Technologies, Cat #31985-062) to 2 mL. 60 uL of transfection reagent (X-tremeGENEHP Roche, Cat #06 366 236 001) was added, and the tube was vortexed and incubated at room temperature for 20 minutes. The transfection reagent/DNA mixture was added to the cells in the T75 flask, and the cells were incubated at 37° C. in 5% CO₂ for 18 hours. The next day, the transfected cells were resuspended in EMEM media with 10% CSS and plated in a 96 well plate at 80,000 cells/well, 100 uL/well. Cells were incubated at 37° C. in 5% CO₂ for 18 hours.

### Test Compounds

Compounds were serially diluted in DMSO (1000× final concentration) and then further diluted in EMEM media with 10% CSS to a 20× final concentration. 10 uL of the 20× compound mix was added to each well of the transfected cells, and the cells were incubated at 37° C. in 5% CO₂ for 30 minutes. 100 uL of EMEM media with 60 uM of estradiol (Sigma cat #E8875) was added, and the cells were incubated for 4 hours at 37° C. in 5% CO₂. The cell media was examined for estradiol and estrone concentrations by LCMS.

### Example B-6: NASH Activity Study (AMLN Model)

NASH is induced in male C57BL/6 mice by diet-induction with AMLN diet (DIO-NASH) (D09100301, Research Diet, USA) (40% fat (18% trans-fat), 40% carbohydrates (20% fructose) and 2% cholesterol). The animals are kept on the diet for 29 weeks. After 26 weeks of diet induction, liver biopsies are performed for base line histological assessment of disease progression (hepatosteatosis and fibrosis), stratified and randomized into treatment groups according to liver fibrosis stage, steatosis score, and body weight. Three weeks after biopsy the mice are stratified into treatment groups and dosed daily by oral gavage with an HSD17B13 inhibitor for 8 weeks. At the end of the study liver biopsies are performed to assess hepatic steatosis and fibrosis by examining tissue sections stained with H&E and Sirius Red, respectively. Total collagen content in the liver is measured by colorimetric determination of hydroxyproline residues by acid hydrolysis of collagen. Triglycerides and total cholesterol content in liver homogenates are measured in single determinations using autoanalyzer Cobas C-111 with commercial kit (Roche Diagnostics, Germany) according to manufacturer's instructions.

### Example B-7: CCl₄ Fibrosis Model

Fibrosis is induced in C57BL/6 male mice by bi-weekly oral administration of CCl₄. CCl₄ is formulated 1:4 in oil and is oral dosed at a final concentration of 0.5 ul/g mouse. After 2-4 weeks of fibrosis induction the compounds is administered daily by oral gavage for 2-8 weeks of treatment while continuing CCl₄ administration. At study termination livers are formalin fixed and stained with H&E or Sirius Red stain for histopathological evaluation of inflammation and fibrosis. Total collagen content is measured by colorimetric determination of hydroxyproline residues by acid hydrolysis of collagen. Collagen gene induction is measured by qPCR analysis of Col1a1 and Col3a1 mRNA. Serum alanine aminotransferase (ALT) and aspartate aminotransferase (AST) are measured by a clinical chemistry analyzer.

### Example B-8: Mouse PK Study

The plasma pharmacokinetics of any one of the compounds disclosed herein as a test article is measured following a single bolus intravenous and oral administration to mice (CD-1, C57BL, and diet induced obesity mice). Test article is formulated for intravenous administration in a vehicle solution of DMSO, PEG400, hydroxypropyl-β-cyclodextrin (HPPCD) and is administered (for example at a dose volume of 3 mL/kg) at selected dose levels. An oral dosing formulation is prepared in appropriate oral dosing vehicles (vegetable oils, PEG400, Solutol, citrate buffer, or carboxymethyl cellulose) and is administered at a dose volume of 5˜10 mL/kg at selected dose levels. Blood samples (approximately 0.15 mL) are collected by cheek pouch method at pre-determined time intervals post intravenous or oral doses into tubes containing EDTA. Plasma is isolated by centrifugation of blood at 10,000 g for 5 minutes, and aliquots are transferred into a 96-well plate and stored at −60° C. or below until analysis.

Calibration standards of test article are prepared by diluting DMSO stock solution with DMSO in a concentration range. Aliquots of calibration standards in DMSO are combined with plasma from naïve mouse so that the final concentrations of calibration standards in plasma are 10-fold lower than the calibration standards in DMSO. PK plasma samples are combined with blank DMSO to match the matrix. The calibration standards and PK samples are combined with ice-cold acetonitrile containing an analytical internal standard and centrifuged at 1850 g for 30 minutes at 4° C. The supernatant fractions are analyzed by LC/MS/MS and quantitated against the calibration curve. Pharmacokinetic parameters (area under the curve (AUC), Cₘₐₓ, Tₘₐₓ, elimination half-life (T_{1/2}), clearance (CL), steady state volume of distribution (V_{dss}), and mean residence time (MRT)) are calculated via non-compartmental analysis using Microsoft Excel (version 2013).

### Example B-9: Mouse CDA-HFD NASH Model

A NASH phenotype with mild fibrosis can be induced in C57BL/6 mice by feeding a choline-deficient diet with 0.1% methionine and 60% kcal fat (Research Diet A06071302) for 4-12 weeks. After 4-6 weeks of diet induction compounds can be administered daily by oral gavage for 4-8 weeks of treatment while continuing CDA-HFD feeding. At study termination livers can be formalin fixed and stained with H&E and Sirius Red stain histopathological evaluation of steatosis, inflammation, and fibrosis. Total collagen content can be measured by colorimetric determination of hydroxyproline residues by acid hydrolysis of collagen. Collagen gene induction can be measured by qPCR analysis of Col1a1 or Col3a1. Serum alanine aminotransferase (ALT) and aspartate aminotransferase (AST) can be measured by a clinical chemistry analyzer.

What is claimed is:

1. A compound of Formula (I), or a pharmaceutically acceptable salt or solvate thereof: 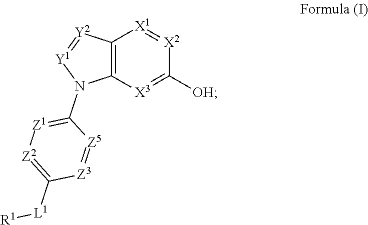
wherein:
X¹, X², and X³ are each independently CR³ or N;
Y¹ and Y² are each independently CR⁴ or N;
Z¹ is CR⁵ or N;
Z² is CR⁵ or N;
Z³ is CR⁵ or N;
Z⁴ is CR⁵;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁴ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹), —S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

2. The compound of claim 1, or a pharmaceutically acceptable salt or solvate thereof, wherein X¹, X², and X³ are CR³.

3. The compound of claim 1 or 2, or a pharmaceutically acceptable salt or solvate thereof, wherein Y² is CR⁴.

4. The compound of any one of claims 1-3, or a pharmaceutically acceptable salt or solvate thereof, having the structure of Formula (Ia):

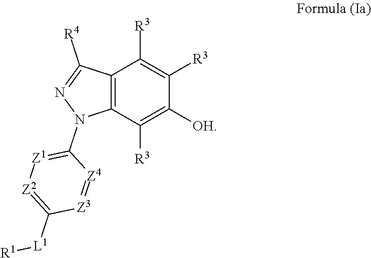

5. The compound of claim 1 or 2, or a pharmaceutically acceptable salt or solvate thereof, wherein Y² is N.

6. The compound of claim 5, or a pharmaceutically acceptable salt or solvate thereof, having the structure of Formula (Ib):

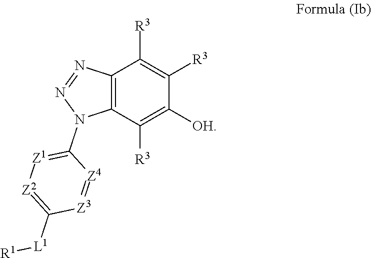

7. A compound of Formula (II), or a pharmaceutically acceptable salt or solvate thereof: 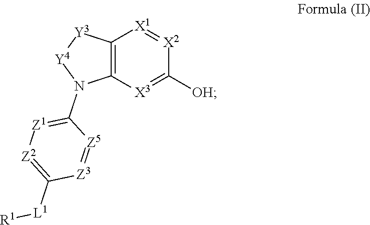
wherein:
X¹, X², and X³ are each independently CR³ or N;
Y³ is C(O), C(R⁴)₂, N(R⁶), O, or S;
Y⁴ is C(O), C(R⁴)₂, or N(R⁶), wherein at least one of Y³ and Y⁴ is C(O);
Z¹ is CR⁵ or N;
Z² is CR⁵ or N;
Z³ is CR⁵ or N;
Z⁵ is CR⁵ or N;
L¹ is selected from a bond, —O—, —S—, —N(R¹²)—, —C(O)—, —S(O)—, —S(O)₂—, —C(R¹⁰)(R¹¹)N(R¹⁰)—, and —N(R¹²)C(R¹⁰)(R¹¹)—;
R¹ is selected from C₃₋₈cycloalkyl, C₂₋₉heterocycloalkyl, C₆₋₁₀aryl and C₁₋₉heteroaryl, wherein C₃₋₈cycloalkyl, C₂₋₉heterocycloalkyl, C₆₋₁₀aryl, and C₁₋₉heteroaryl are optionally substituted with one, two, three, four, or five R²;
each R² is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R³ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁴ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, —CN, C₁₋₆alkyl, C₁₋₆haloalkyl, —OR¹⁰, and —N(R¹⁰)(R¹¹);
each R⁵ is independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, C₁₋₆alkyl, C₁₋₆haloalkyl, —OR¹⁰, and —N(R¹⁰)(R¹¹);
R⁶ is selected from hydrogen, C₁₋₆alkyl, C₁₋₆ haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —C(O)R¹³, —S(O)R¹³, —S(O)₂R¹³, —C(O)OR¹⁰, —S(O)₂OR¹⁰, —C(O)N(R¹⁰)(R¹¹), —S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, C₁₋₆alkyl, C₁₋₆haloalkyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —C(O)N(R¹⁰)(R¹¹), —S(O)R¹³, —S(O)₂R¹³, —S(O)₂N(R¹⁰)(R¹¹), —OC(O)N(R¹⁰)(R¹¹), —N(R¹²)C(O)R¹³, —N(R¹²)C(O)OR¹³, and —N(R¹²)C(O)N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

8. The compound of claim 7, or a pharmaceutically acceptable salt or solvate thereof, wherein X¹, X², and X³ are CR³.

9. The compound of claim 7 or claim 8, or a pharmaceutically acceptable salt or solvate thereof, having the structure of Formula (IIa):

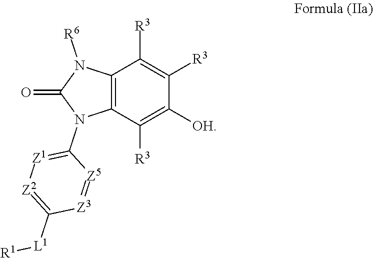

10. The compound of any one of claims 7-9, or a pharmaceutically acceptable salt or solvate thereof, wherein R⁶ is selected from hydrogen and C₁₋₆alkyl optionally substituted with one, two, or three groups selected from halogen, —CN, C₁₋₆alkyl, C₁₋₆ haloalkyl, —OR¹⁰, and —N(R¹⁰)(R¹¹).

11. The compound of any one of claims 7-10, or a pharmaceutically acceptable salt or solvate thereof, wherein R⁶ is C₁₋₆alkyl optionally substituted with —OH.

12. The compound of claim 7 or claim 8, or a pharmaceutically acceptable salt or solvate thereof, having the structure of Formula (IIb):

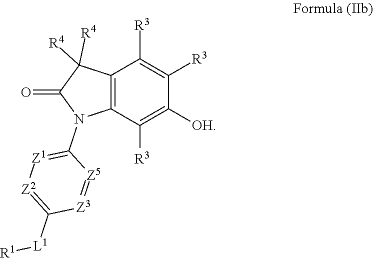

13. The compound of any one of claims 7-12, or a pharmaceutically acceptable salt or solvate thereof, wherein Z⁵ is CR⁵.

14. The compound of any one of claims 7-12, or a pharmaceutically acceptable salt or solvate thereof, wherein Z⁵ is N.

15. The compound of any one of claims 1-14, or a pharmaceutically acceptable salt or solvate thereof, wherein Z¹, Z², and Z³ are CR⁵.

16. The compound of any one of claims 1-14, or a pharmaceutically acceptable salt or solvate thereof, wherein Z¹ is N; and Z² and Z³ are CR⁵.

17. The compound of any one of claims 1-14, or a pharmaceutically acceptable salt or solvate thereof, wherein Z³ is N; and Z¹ and Z² are CR⁵.

18. The compound of any one of claims 1-14, or a pharmaceutically acceptable salt or solvate thereof, wherein Z¹ is CR⁵; and Z² and Z³ are N.

19. The compound of any one of claims 1-14, or a pharmaceutically acceptable salt or solvate thereof, wherein Z² is CR⁵; and Z¹ and Z³ are N.

20. The compound of any one of claims 1-14, or a pharmaceutically acceptable salt or solvate thereof, wherein Z³ is CR⁵; and Z¹ and Z² are N.

21. The compound of any one of claims 1-20, or a pharmaceutically acceptable salt or solvate thereof, wherein L¹ is a bond.

22. The compound of any one of claims 1-21, or a pharmaceutically acceptable salt or solvate thereof, wherein R¹ is C₂₋₉heterocycloalkyl optionally substituted with one, two, three, four, or five R².

23. The compound of any one of claims 1-22, or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, and aziridinyl, wherein piperidinyl, piperazinyl, morpholinyl, tetrahydropyranyl, tetrahydrofuranyl, pyrrolidinyl, oxetanyl, azetidinyl, aziridinyl, azepanyl, and diazepanyl are optionally substituted with one, two, or three R².

24. The compound of any one of claims 1-22, or a pharmaceutically acceptable salt or solvate thereof, wherein R¹ is C₂₋₉heterocycloalkyl selected from thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane, 2,6-diazaspiro[3.3]heptane, and 2-oxa-6-azaspiro[3.3]heptane, wherein thiomorpholine, 1,4-diazepane, 3,6-diazabicyclo[3.1.1]heptane, 6-oxa-3-azabicyclo[3.1.1]heptane, 2-oxa-7-azaspiro[3.5]nonane, 7-oxa-2-azaspiro[3.5]nonane, 7-azaspiro[3.5]nonane, 6-azaspiro[2.5]octane, 2,7-diazaspiro[3.5]nonane, 2,6-diazaspiro[3.3]heptane, and 2-oxa-6-azaspiro[3.3]heptane are optionally substituted with one, two, or three R².

25. The compound of claim 24, or a pharmaceutically acceptable salt or solvate thereof, wherein R¹ is C₂₋₉heterocycloalkyl selected from thiomorpholine, 2-oxa-7-azaspiro[3.5]nonane, 7-oxa-2-azaspiro[3.5]nonane, 2,7-diazaspiro[3.5]nonane, 2,6-diazaspiro[3.3]heptane, wherein thiomorpholine, 2-oxa-7-azaspiro[3.5]nonane, 7-oxa-2-azaspiro[3.5]nonane, 2,7-diazaspiro[3.5]nonane, and 2,6-diazaspiro[3.3]heptane are optionally substituted with one, two, or three R².

26. The compound of any one of claims 1-22, or a pharmaceutically acceptable salt or solvate thereof, wherein R¹ is

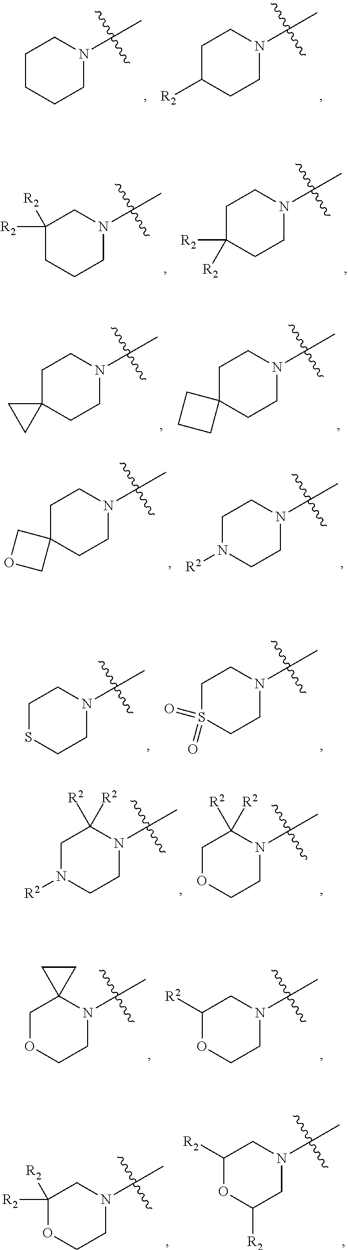

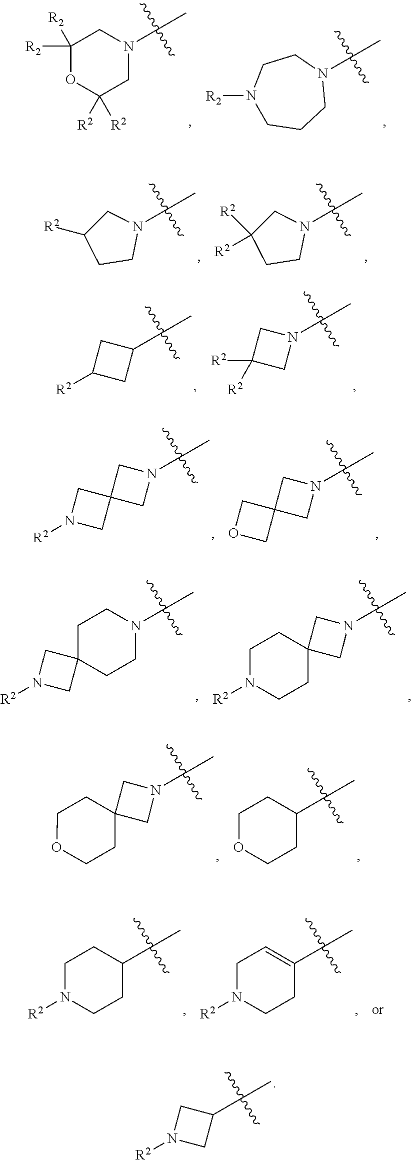

27. The compound of any one of claims 1-26, or a pharmaceutically acceptable salt or solvate thereof, wherein each R² is independently selected from C₁₋₆alkyl, C₁₋₆ haloalkyl, —CN, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—.

28. The compound of any one of claims 1-27, or a pharmaceutically acceptable salt or solvate thereof, wherein R¹ is

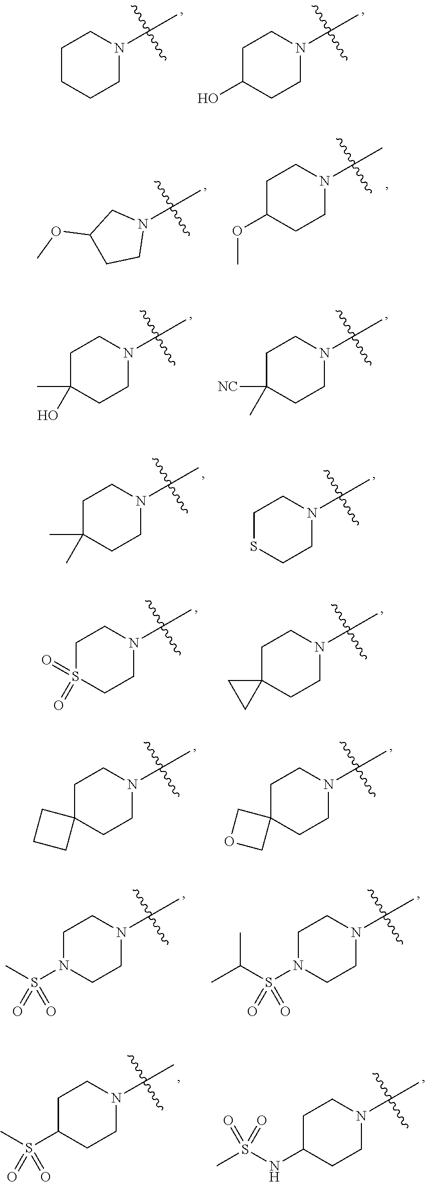

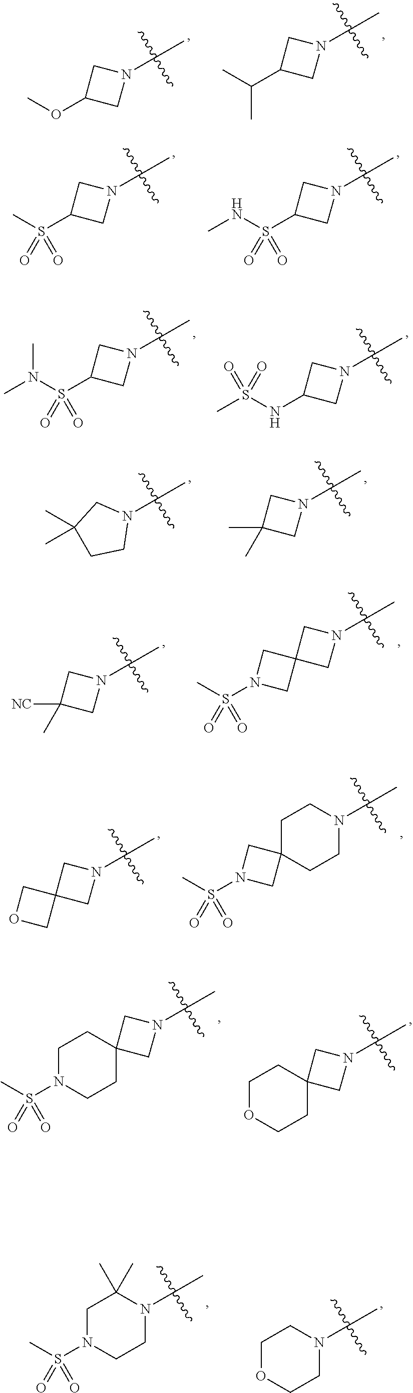

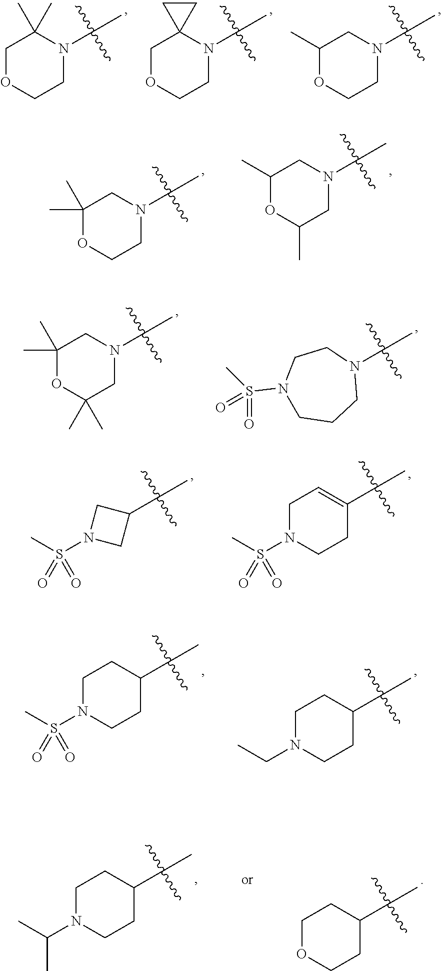

29. The compound of any one of claims 1-21, or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R².

30. The compound of claim 29, or a pharmaceutically acceptable salt or solvate thereof, wherein R¹ is phenyl substituted with one, two, or three R².

31. The compound of claim 29, or a pharmaceutically acceptable salt or solvate thereof, wherein R¹ is C₁₋₉heteroaryl substituted with one, two, or three R².

32. The compound of claim 31, or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R².

33. The compound of any one of claims 29-32, or a pharmaceutically acceptable salt or solvate thereof, wherein each R² is independently selected from halogen, —CN, C₁₋₆ alkyl, C₁₋₆ haloalkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹).

34. The compound of any one of claims 29-33, or a pharmaceutically acceptable salt or solvate thereof, wherein each R² is independently selected from halogen, —CN, C₁₋₆ alkyl, —OH, —N(H)S(O)₂CH₃, —S(O)₂CH₃, and —S(O)₂NH₂.

35. The compound of any one of claims 1-34, or a pharmaceutically acceptable salt or solvate thereof, wherein each R⁵ is independently selected from hydrogen, halogen, —CN, C₁₋₆alkyl, C₁₋₆ haloalkyl, and —OR¹⁰.

36. The compound of any one of claims 1-35, or a pharmaceutically acceptable salt or solvate thereof, wherein each R⁵ is hydrogen.

37. The compound of any one of claims 1-36, or a pharmaceutically acceptable salt or solvate thereof, wherein each R⁴ is independently selected from hydrogen, halogen, —CN, C₁₋₆alkyl, C₁₋₆ haloalkyl, and C₃₋₆cycloalkyl.

38. The compound of any one of claims 1-37, or a pharmaceutically acceptable salt or solvate thereof, wherein each R⁴ is hydrogen.

39. The compound of any one of claims 1-38, or a pharmaceutically acceptable salt or solvate thereof, wherein each R³ is independently selected from hydrogen, halogen, —CN, C₁₋₆alkyl, C₁₋₆haloalkyl, and —OH.

40. The compound of any one of claims 1-39, or a pharmaceutically acceptable salt or solvate thereof, wherein each R³ is independently selected from hydrogen, halogen, —CN, and CF₃.

41. A compound selected from: 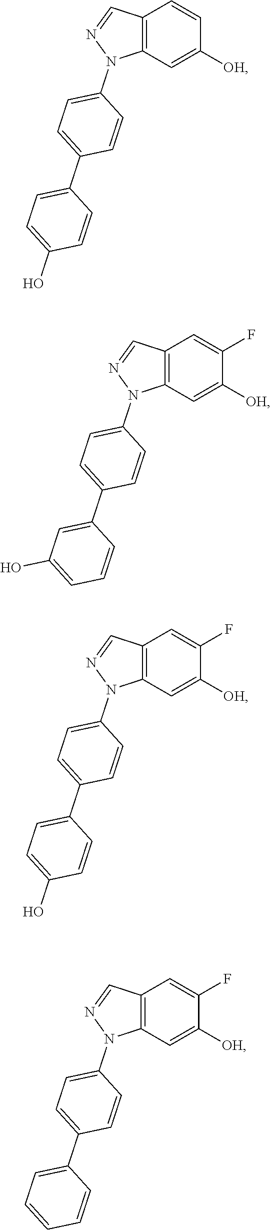 [SMILES: Oc1ccc(-c2ccc(-n3ncc4ccc(O)cc43)cc2)cc1 ; Oc1cccc(-c2ccc(-n3ncc4cc(F)c(O)cc43)cc2)c1 ; Oc1ccc(-c2ccc(-n3ncc4cc(F)c(O)cc43)cc2)cc1 ; Oc1cc2c(cnn2-c2ccc(-c3ccccc3)cc2)cc1F] 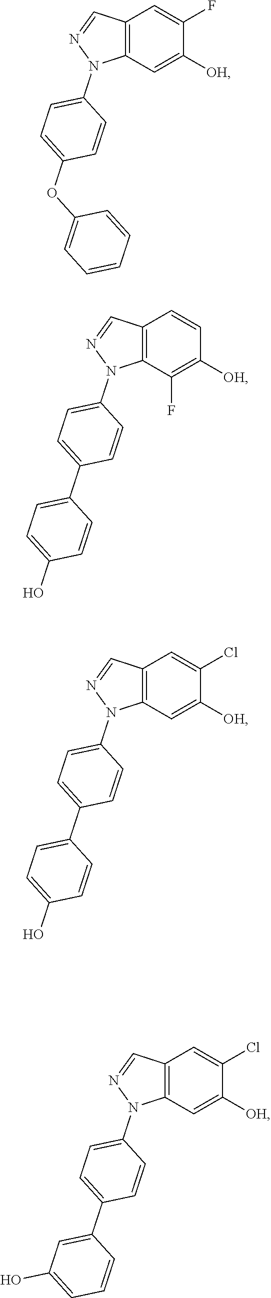 [SMILES: Oc1cc2c(cnn2-c2ccc(Oc3ccccc3)cc2)cc1F ; Oc1ccc(-c2ccc(-n3ncc4ccc(O)c(F)c43)cc2)cc1 ; Oc1ccc(-c2ccc(-n3ncc4cc(Cl)c(O)cc43)cc2)cc1 ; Oc1cccc(-c2ccc(-n3ncc4cc(Cl)c(O)cc43)cc2)c1] 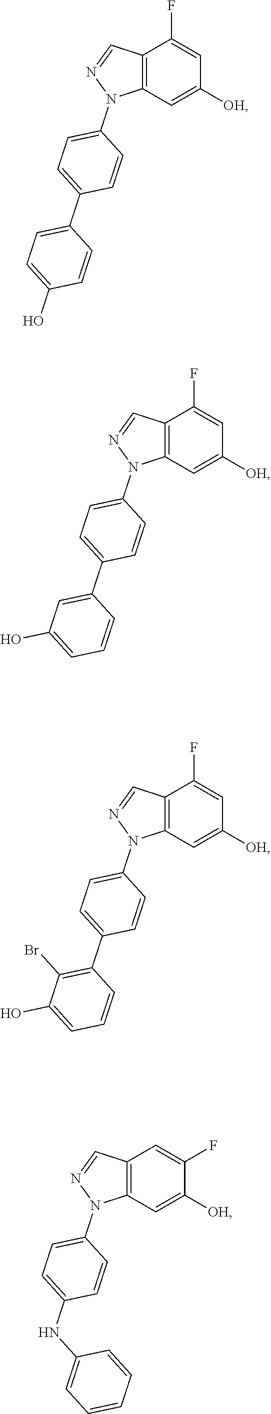 [SMILES: Oc1ccc(-c2ccc(-n3ncc4c(F)cc(O)cc43)cc2)cc1 ; Oc1cccc(-c2ccc(-n3ncc4c(F)cc(O)cc43)cc2)c1 ; Oc1cc(F)c2cnn(-c3ccc(-c4cccc(O)c4Br)cc3)c2c1 ; Oc1cc2c(cnn2-c2ccc(Nc3ccccc3)cc2)cc1F] 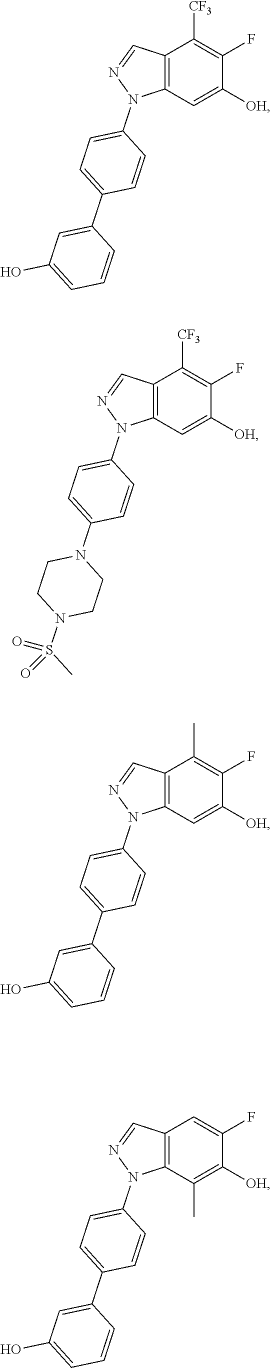 [SMILES: Oc1cccc(-c2ccc(-n3ncc4c(C(F)(F)F)c(F)c(O)cc43)cc2)c1 ; CS(=O)(=O)N1CCN(c2ccc(-n3ncc4c(C(F)(F)F)c(F)c(O)cc43)cc2)CC1 ; Cc1c(F)c(O)cc2c1cnn2-c1ccc(-c2cccc(O)c2)cc1 ; Cc1c(O)c(F)cc2cnn(-c3ccc(-c4cccc(O)c4)cc3)c12] 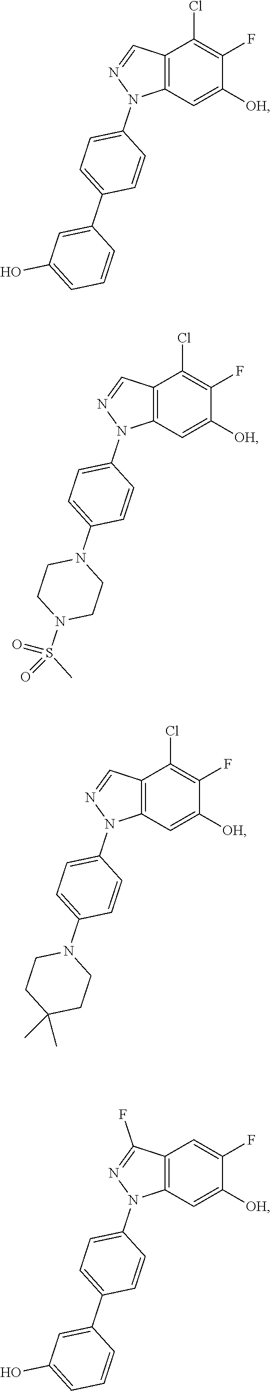 [SMILES: Oc1cccc(-c2ccc(-n3ncc4c(Cl)c(F)c(O)cc43)cc2)c1 ; CS(=O)(=O)N1CCN(c2ccc(-n3ncc4c(Cl)c(F)c(O)cc43)cc2)CC1 ; CC1(C)CCN(c2ccc(-n3ncc4c(Cl)c(F)c(O)cc43)cc2)CC1 ; Oc1cccc(-c2ccc(-n3nc(F)c4cc(F)c(O)cc43)cc2)c1] 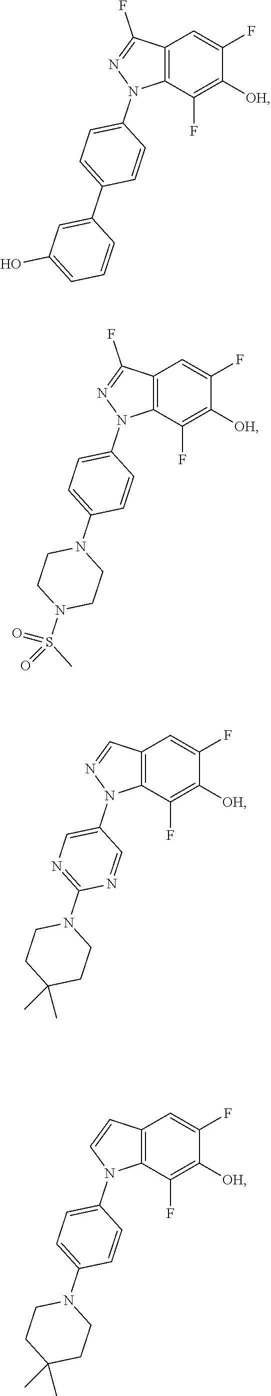 [SMILES: Oc1cccc(-c2ccc(-n3nc(F)c4cc(F)c(O)c(F)c43)cc2)c1 ; CS(=O)(=O)N1CCN(c2ccc(-n3nc(F)c4cc(F)c(O)c(F)c43)cc2)CC1 ; CC1(C)CCN(c2ncc(-n3ncc4cc(F)c(O)c(F)c43)cn2)CC1 ; CC1(C)CCN(c2ccc(-n3ccc4cc(F)c(O)c(F)c43)cc2)CC1] 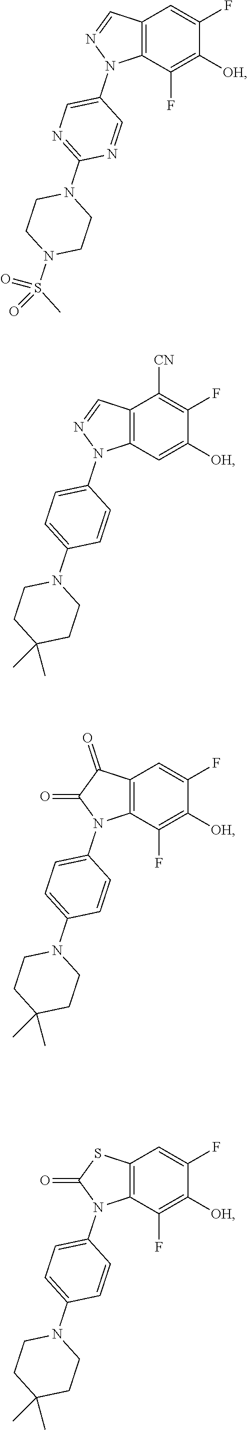 [SMILES: CS(=O)(=O)N1CCN(c2ncc(-n3ncc4cc(F)c(O)c(F)c43)cn2)CC1 ; CC1(C)CCN(c2ccc(-n3ncc4c(C#N)c(F)c(O)cc43)cc2)CC1 ; CC1(C)CCN(c2ccc(N3C(=O)C(=O)c4cc(F)c(O)c(F)c43)cc2)CC1 ; CC1(C)CCN(c2ccc(-n3c(=O)sc4cc(F)c(O)c(F)c43)cc2)CC1] 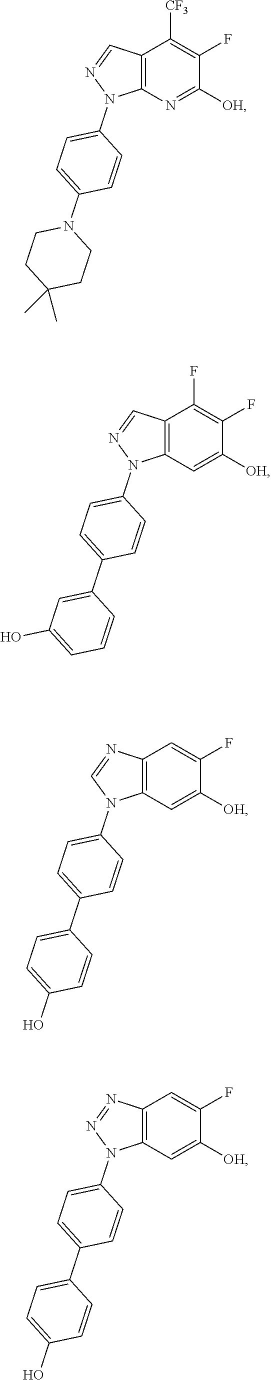 [SMILES: CC1(C)CCN(c2ccc(-n3ncc4c(C(F)(F)F)c(F)c(O)nc43)cc2)CC1 ; Oc1cccc(-c2ccc(-n3ncc4c(F)c(F)c(O)cc43)cc2)c1 ; Oc1ccc(-c2ccc(-n3cnc4cc(F)c(O)cc43)cc2)cc1 ; Oc1ccc(-c2ccc(-n3nnc4cc(F)c(O)cc43)cc2)cc1] 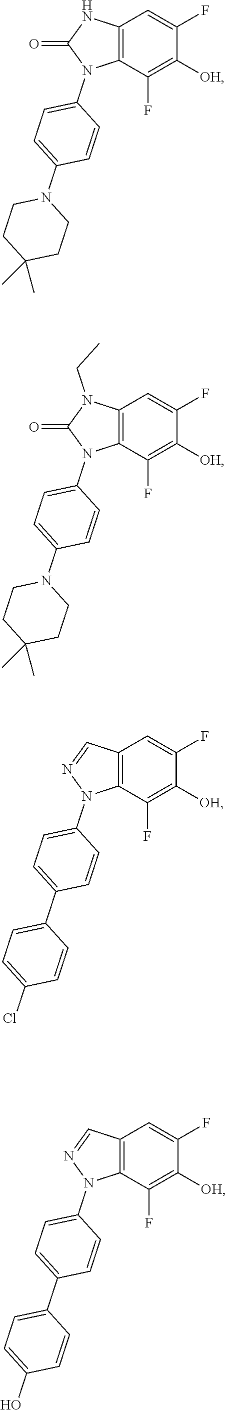 [SMILES: CC1(C)CCN(c2ccc(-n3c(=O)[nH]c4cc(F)c(O)c(F)c43)cc2)CC1 ; CCn1c(=O)n(-c2ccc(N3CCC(C)(C)CC3)cc2)c2c(F)c(O)c(F)cc21 ; Oc1c(F)cc2cnn(-c3ccc(-c4ccc(Cl)cc4)cc3)c2c1F ; Oc1ccc(-c2ccc(-n3ncc4cc(F)c(O)c(F)c43)cc2)cc1] 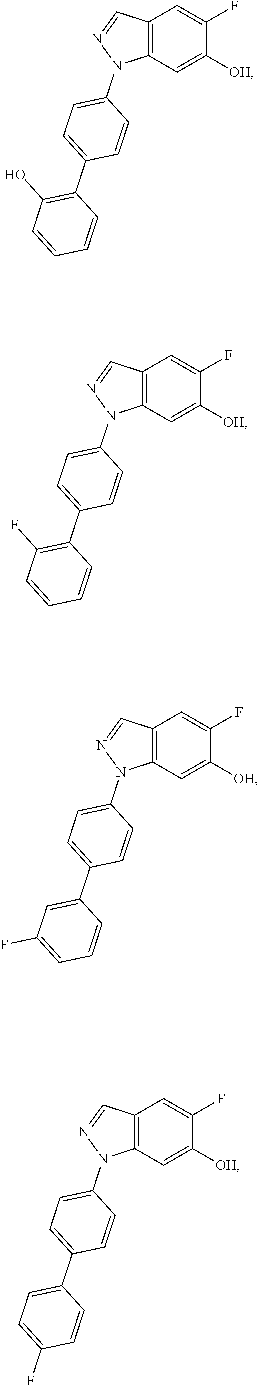 [SMILES: Oc1cc2c(cnn2-c2ccc(-c3ccccc3O)cc2)cc1F ; Oc1cc2c(cnn2-c2ccc(-c3ccccc3F)cc2)cc1F ; Oc1cc2c(cnn2-c2ccc(-c3cccc(F)c3)cc2)cc1F ; Oc1cc2c(cnn2-c2ccc(-c3ccc(F)cc3)cc2)cc1F] 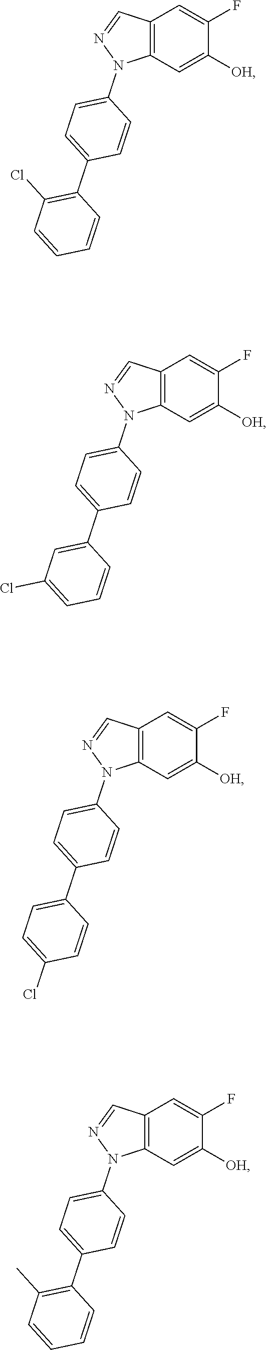 [SMILES: Oc1cc2c(cnn2-c2ccc(-c3ccccc3Cl)cc2)cc1F ; Oc1cc2c(cnn2-c2ccc(-c3cccc(Cl)c3)cc2)cc1F ; Oc1cc2c(cnn2-c2ccc(-c3ccc(Cl)cc3)cc2)cc1F ; Cc1ccccc1-c1ccc(-n2ncc3cc(F)c(O)cc32)cc1] 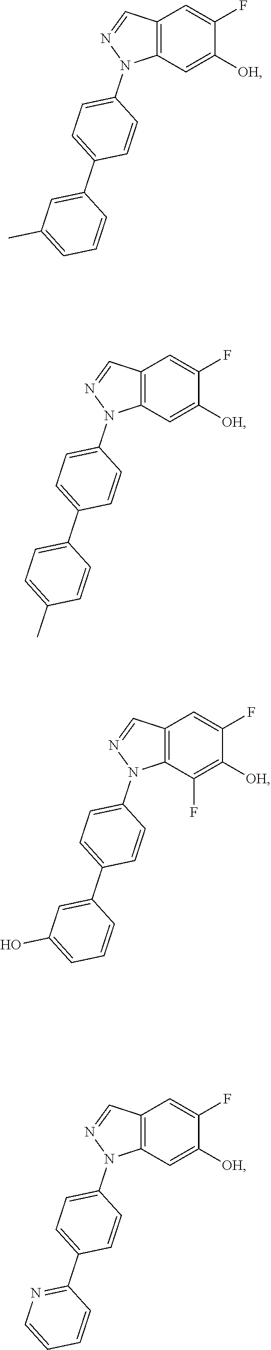 [SMILES: Cc1cccc(-c2ccc(-n3ncc4cc(F)c(O)cc43)cc2)c1 ; Cc1ccc(-c2ccc(-n3ncc4cc(F)c(O)cc43)cc2)cc1 ; Oc1cccc(-c2ccc(-n3ncc4cc(F)c(O)c(F)c43)cc2)c1 ; Oc1cc2c(cnn2-c2ccc(-c3ccccn3)cc2)cc1F] 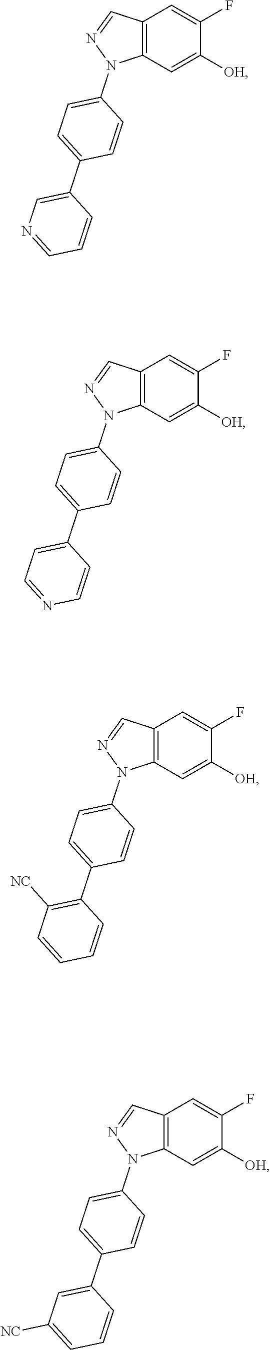 [SMILES: Oc1cc2c(cnn2-c2ccc(-c3cccnc3)cc2)cc1F ; Oc1cc2c(cnn2-c2ccc(-c3ccncc3)cc2)cc1F ; N#Cc1ccccc1-c1ccc(-n2ncc3cc(F)c(O)cc32)cc1 ; N#Cc1cccc(-c2ccc(-n3ncc4cc(F)c(O)cc43)cc2)c1] 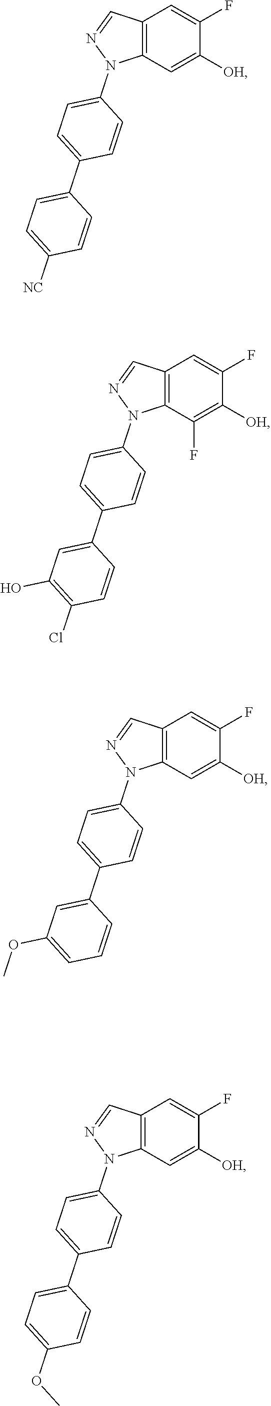 [SMILES: N#Cc1ccc(-c2ccc(-n3ncc4cc(F)c(O)cc43)cc2)cc1 ; Oc1cc(-c2ccc(-n3ncc4cc(F)c(O)c(F)c43)cc2)ccc1Cl ; COc1cccc(-c2ccc(-n3ncc4cc(F)c(O)cc43)cc2)c1 ; COc1ccc(-c2ccc(-n3ncc4cc(F)c(O)cc43)cc2)cc1] 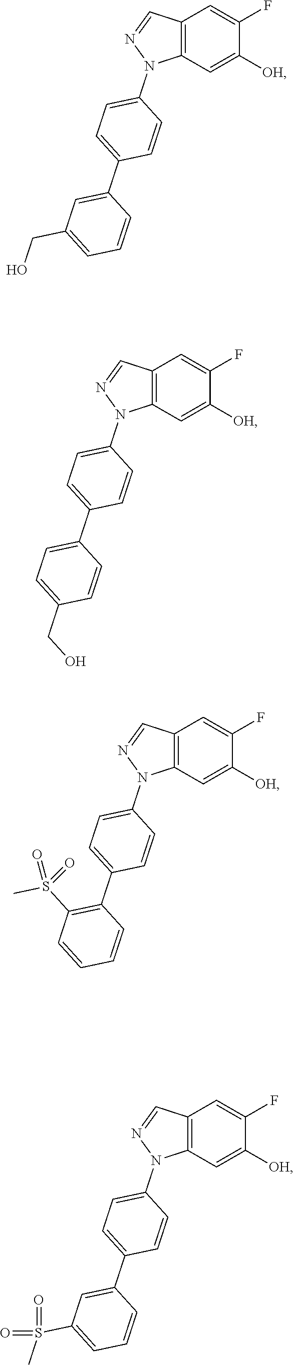 [SMILES: OCc1cccc(-c2ccc(-n3ncc4cc(F)c(O)cc43)cc2)c1 ; OCc1ccc(-c2ccc(-n3ncc4cc(F)c(O)cc43)cc2)cc1 ; CS(=O)(=O)c1ccccc1-c1ccc(-n2ncc3cc(F)c(O)cc32)cc1 ; CS(=O)(=O)c1cccc(-c2ccc(-n3ncc4cc(F)c(O)cc43)cc2)c1] 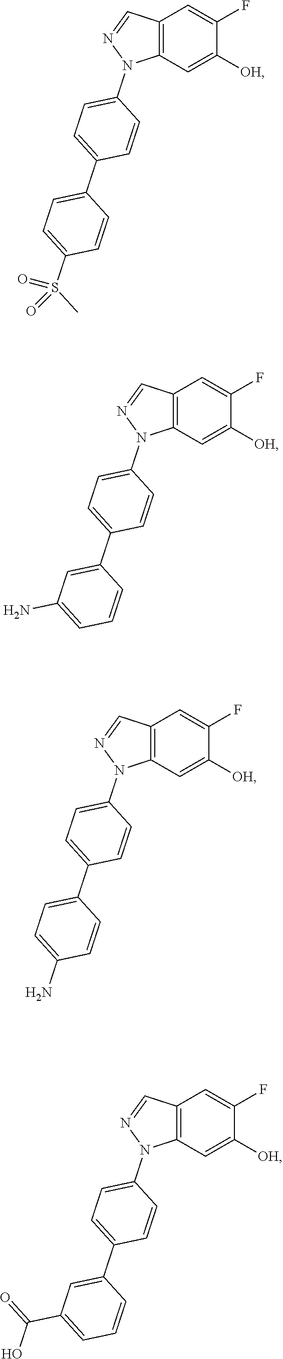 [SMILES: CS(=O)(=O)c1ccc(-c2ccc(-n3ncc4cc(F)c(O)cc43)cc2)cc1 ; Nc1cccc(-c2ccc(-n3ncc4cc(F)c(O)cc43)cc2)c1 ; Nc1ccc(-c2ccc(-n3ncc4cc(F)c(O)cc43)cc2)cc1 ; O=C(O)c1cccc(-c2ccc(-n3ncc4cc(F)c(O)cc43)cc2)c1] 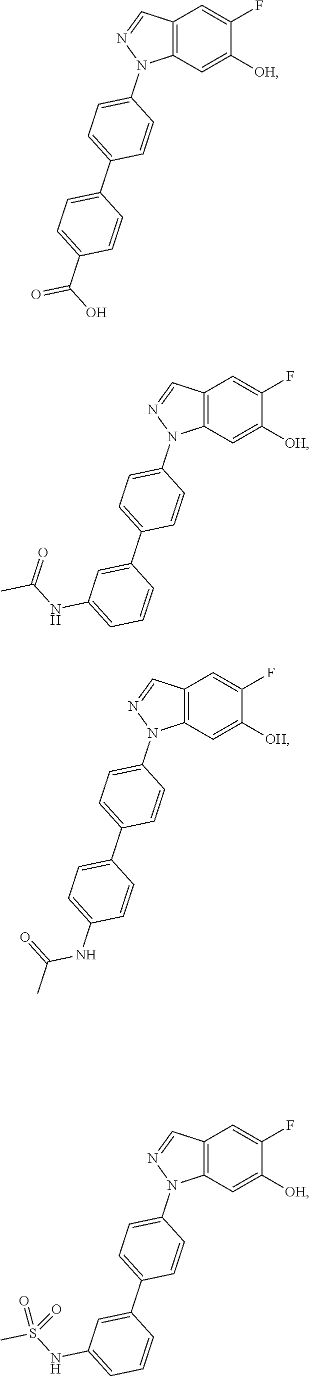 [SMILES: O=C(O)c1ccc(-c2ccc(-n3ncc4cc(F)c(O)cc43)cc2)cc1 ; CC(=O)Nc1cccc(-c2ccc(-n3ncc4cc(F)c(O)cc43)cc2)c1 ; CC(=O)Nc1ccc(-c2ccc(-n3ncc4cc(F)c(O)cc43)cc2)cc1 ; CS(=O)(=O)Nc1cccc(-c2ccc(-n3ncc4cc(F)c(O)cc43)cc2)c1] 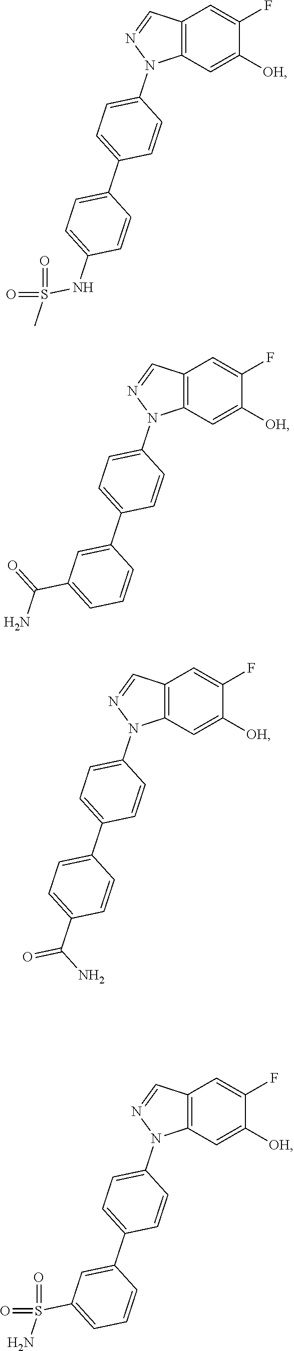 [SMILES: CS(=O)(=O)Nc1ccc(-c2ccc(-n3ncc4cc(F)c(O)cc43)cc2)cc1 ; NC(=O)c1cccc(-c2ccc(-n3ncc4cc(F)c(O)cc43)cc2)c1 ; NC(=O)c1ccc(-c2ccc(-n3ncc4cc(F)c(O)cc43)cc2)cc1 ; NS(=O)(=O)c1cccc(-c2ccc(-n3ncc4cc(F)c(O)cc43)cc2)c1] 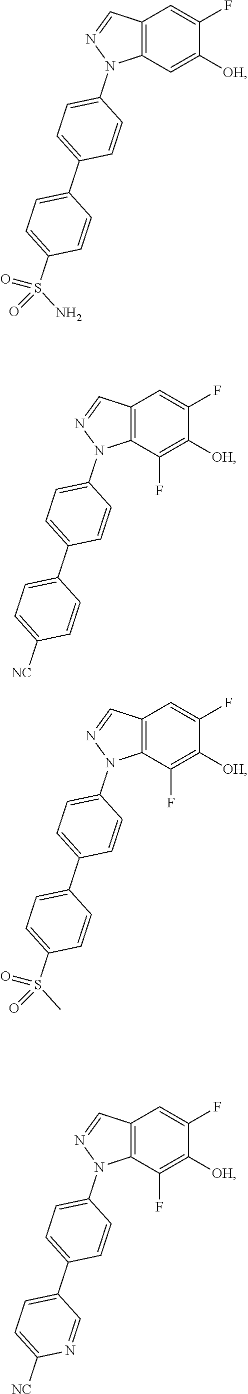 [SMILES: NS(=O)(=O)c1ccc(-c2ccc(-n3ncc4cc(F)c(O)cc43)cc2)cc1 ; N#Cc1ccc(-c2ccc(-n3ncc4cc(F)c(O)c(F)c43)cc2)cc1 ; CS(=O)(=O)c1ccc(-c2ccc(-n3ncc4cc(F)c(O)c(F)c43)cc2)cc1 ; N#Cc1ccc(-c2ccc(-n3ncc4cc(F)c(O)c(F)c43)cc2)cn1] 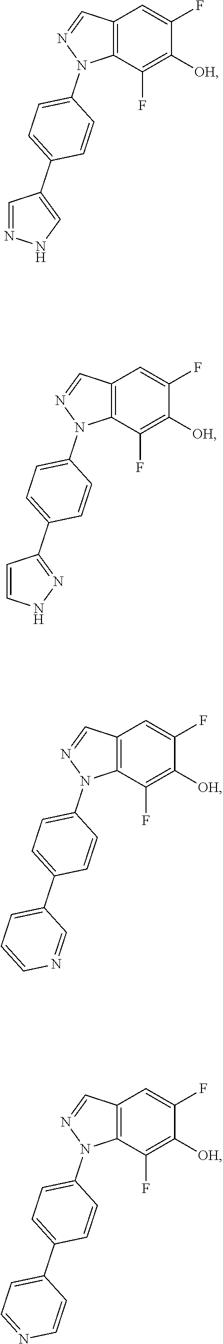 [SMILES: Oc1c(F)cc2cnn(-c3ccc(-c4cn[nH]c4)cc3)c2c1F ; Oc1c(F)cc2cnn(-c3ccc(-c4cc[nH]n4)cc3)c2c1F ; Oc1c(F)cc2cnn(-c3ccc(-c4cccnc4)cc3)c2c1F ; Oc1c(F)cc2cnn(-c3ccc(-c4ccncc4)cc3)c2c1F] 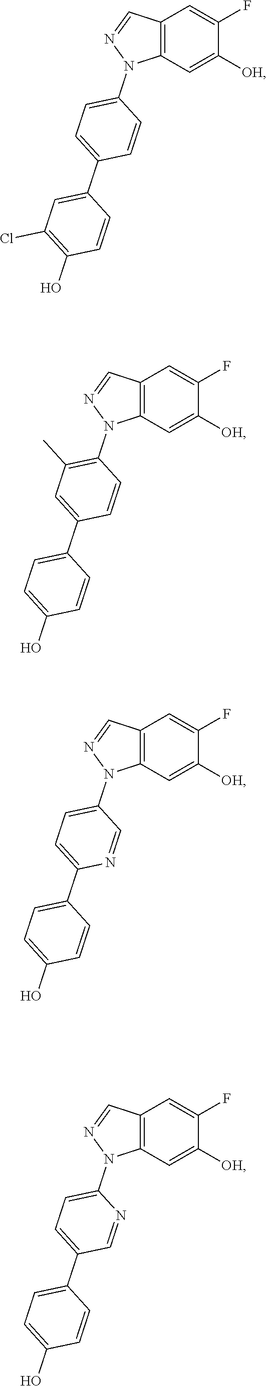 [SMILES: Oc1cc2c(cnn2-c2ccc(-c3ccc(O)c(Cl)c3)cc2)cc1F ; Cc1cc(-c2ccc(O)cc2)ccc1-n1ncc2cc(F)c(O)cc21 ; Oc1ccc(-c2ccc(-n3ncc4cc(F)c(O)cc43)cn2)cc1 ; Oc1ccc(-c2ccc(-n3ncc4cc(F)c(O)cc43)nc2)cc1] 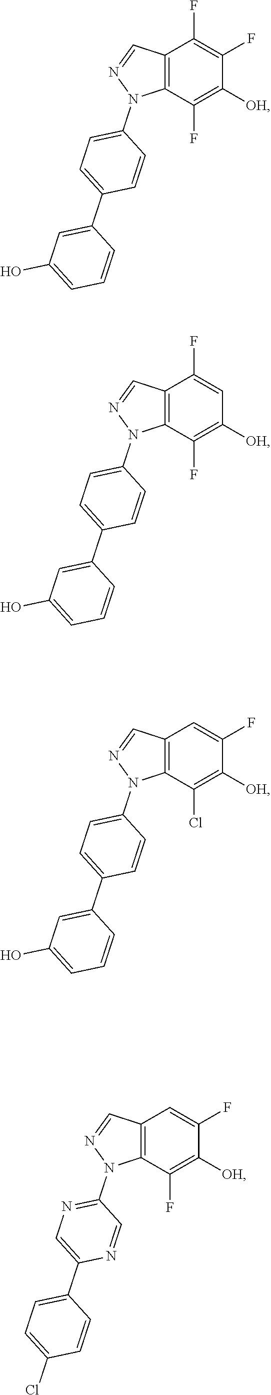 [SMILES: Oc1cccc(-c2ccc(-n3ncc4c(F)c(F)c(O)c(F)c43)cc2)c1 ; Oc1cccc(-c2ccc(-n3ncc4c(F)cc(O)c(F)c43)cc2)c1 ; Oc1cccc(-c2ccc(-n3ncc4cc(F)c(O)c(Cl)c43)cc2)c1 ; Oc1c(F)cc2cnn(-c3cnc(-c4ccc(Cl)cc4)cn3)c2c1F] 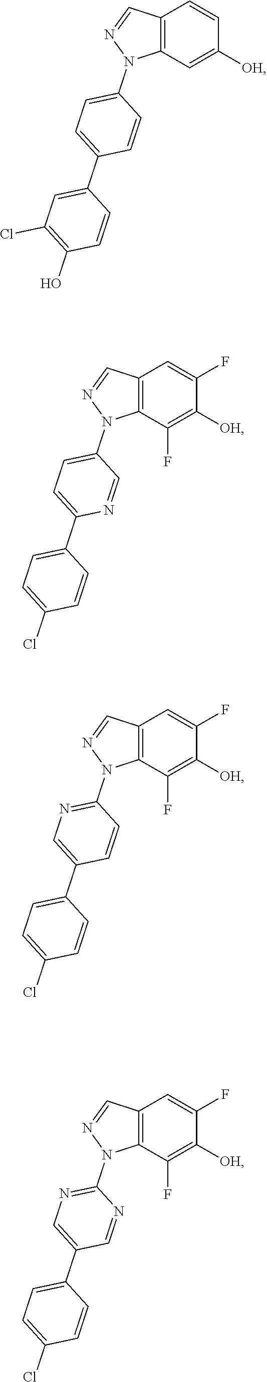 [SMILES: Oc1ccc2cnn(-c3ccc(-c4ccc(O)c(Cl)c4)cc3)c2c1 ; Oc1c(F)cc2cnn(-c3ccc(-c4ccc(Cl)cc4)nc3)c2c1F ; Oc1c(F)cc2cnn(-c3ccc(-c4ccc(Cl)cc4)cn3)c2c1F ; Oc1c(F)cc2cnn(-c3ncc(-c4ccc(Cl)cc4)cn3)c2c1F] 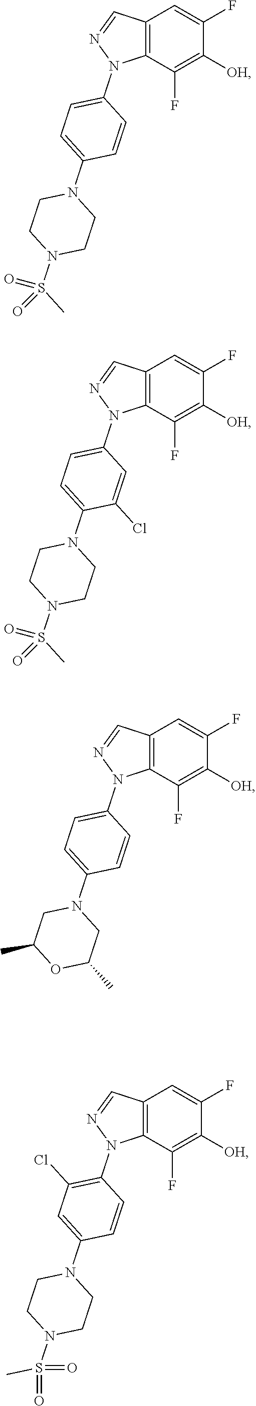 [SMILES: CS(=O)(=O)N1CCN(c2ccc(-n3ncc4cc(F)c(O)c(F)c43)cc2)CC1 ; CS(=O)(=O)N1CCN(c2ccc(-n3ncc4cc(F)c(O)c(F)c43)cc2Cl)CC1 ; C[C@H]1CN(c2ccc(-n3ncc4cc(F)c(O)c(F)c43)cc2)C[C@H](C)O1 ; CS(=O)(=O)N1CCN(c2ccc(-n3ncc4cc(F)c(O)c(F)c43)c(Cl)c2)CC1] 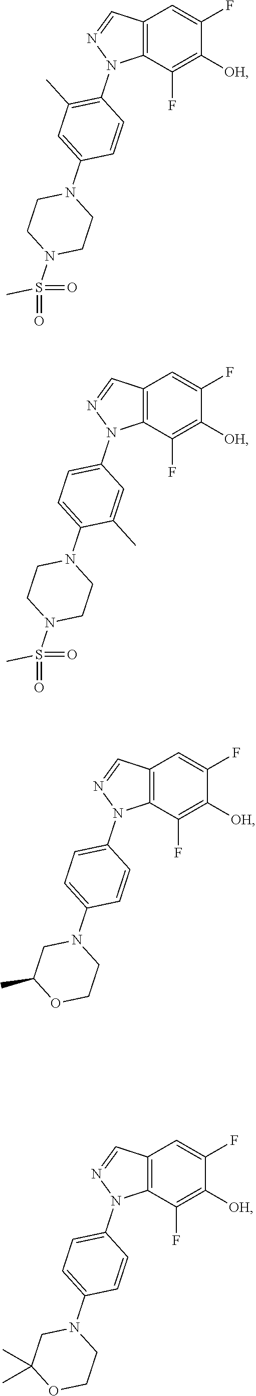 [SMILES: Cc1cc(N2CCN(S(C)(=O)=O)CC2)ccc1-n1ncc2cc(F)c(O)c(F)c21 ; Cc1cc(-n2ncc3cc(F)c(O)c(F)c32)ccc1N1CCN(S(C)(=O)=O)CC1 ; C[C@H]1CN(c2ccc(-n3ncc4cc(F)c(O)c(F)c43)cc2)CCO1 ; CC1(C)CN(c2ccc(-n3ncc4cc(F)c(O)c(F)c43)cc2)CCO1] 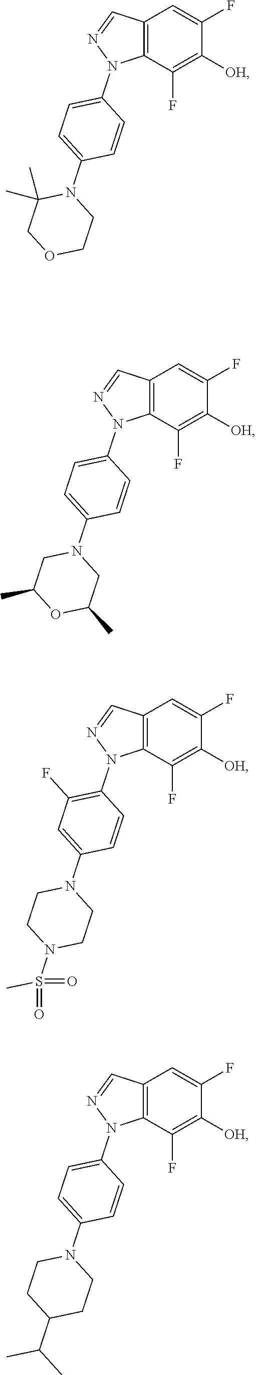 [SMILES: CC1(C)COCCN1c1ccc(-n2ncc3cc(F)c(O)c(F)c32)cc1 ; C[C@@H]1CN(c2ccc(-n3ncc4cc(F)c(O)c(F)c43)cc2)C[C@H](C)O1 ; CS(=O)(=O)N1CCN(c2ccc(-n3ncc4cc(F)c(O)c(F)c43)c(F)c2)CC1 ; CC(C)C1CCN(c2ccc(-n3ncc4cc(F)c(O)c(F)c43)cc2)CC1] 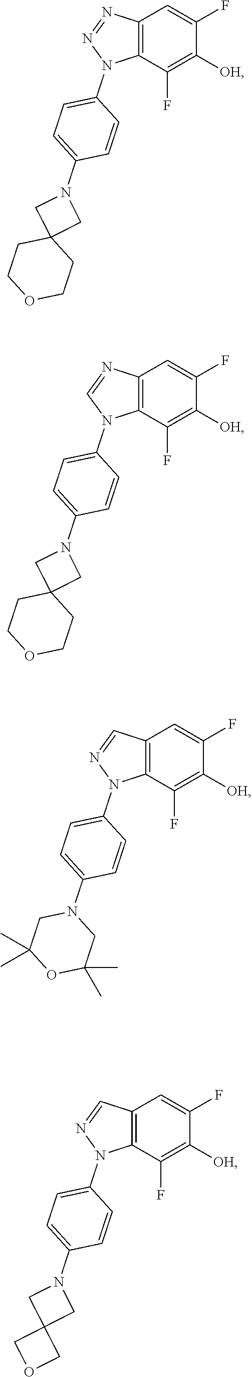 [SMILES: Oc1c(F)cc2nnn(-c3ccc(N4CC5(CCOCC5)C4)cc3)c2c1F ; Oc1c(F)cc2ncn(-c3ccc(N4CC5(CCOCC5)C4)cc3)c2c1F ; CC1(C)CN(c2ccc(-n3ncc4cc(F)c(O)c(F)c43)cc2)CC(C)(C)O1 ; Oc1c(F)cc2cnn(-c3ccc(N4CC5(COC5)C4)cc3)c2c1F] 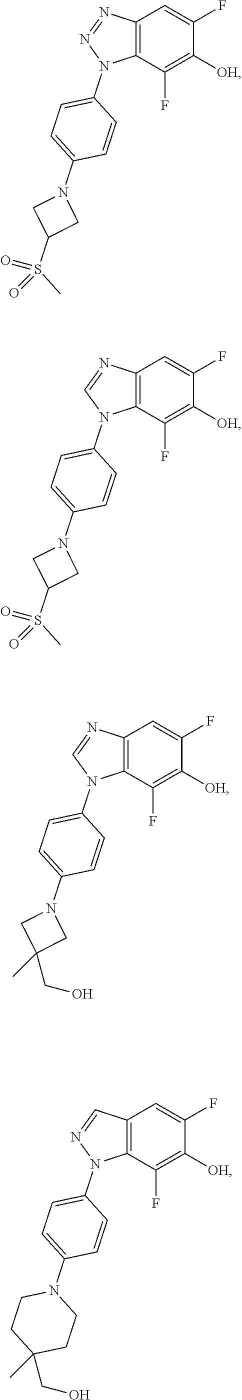 [SMILES: CS(=O)(=O)C1CN(c2ccc(-n3nnc4cc(F)c(O)c(F)c43)cc2)C1 ; CS(=O)(=O)C1CN(c2ccc(-n3cnc4cc(F)c(O)c(F)c43)cc2)C1 ; CC1(CO)CN(c2ccc(-n3cnc4cc(F)c(O)c(F)c43)cc2)C1 ; CC1(CO)CCN(c2ccc(-n3ncc4cc(F)c(O)c(F)c43)cc2)CC1] 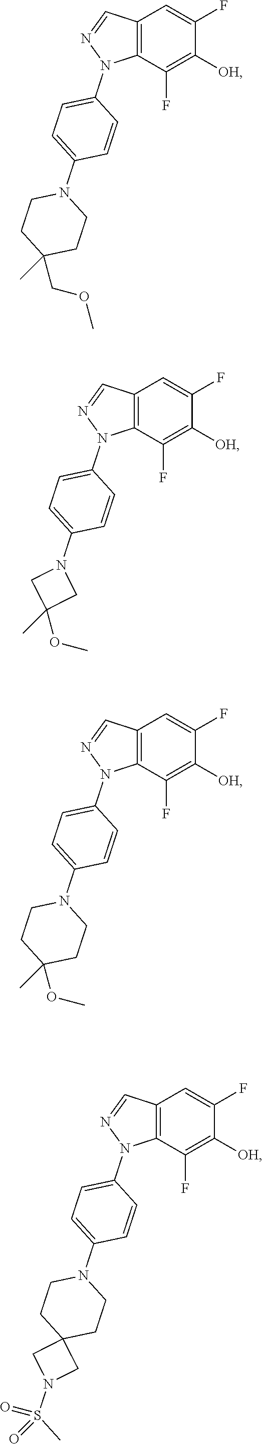 [SMILES: COCC1(C)CCN(c2ccc(-n3ncc4cc(F)c(O)c(F)c43)cc2)CC1 ; COC1(C)CN(c2ccc(-n3ncc4cc(F)c(O)c(F)c43)cc2)C1 ; COC1(C)CCN(c2ccc(-n3ncc4cc(F)c(O)c(F)c43)cc2)CC1 ; CS(=O)(=O)N1CC2(CCN(c3ccc(-n4ncc5cc(F)c(O)c(F)c54)cc3)CC2)C1] 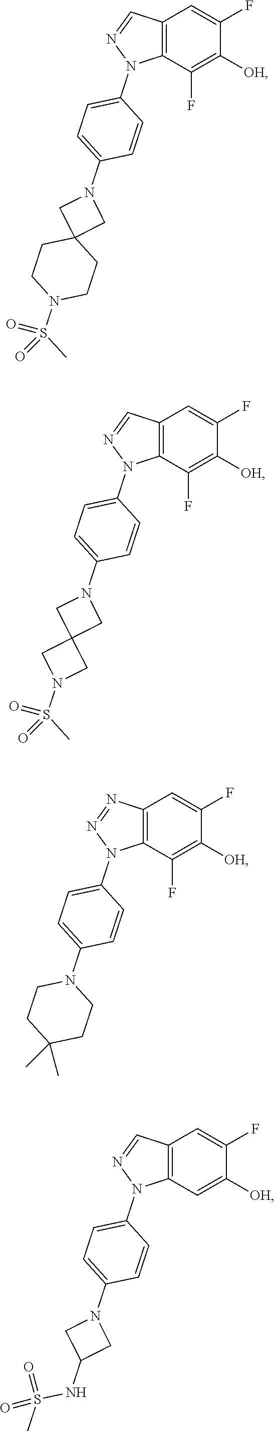 [SMILES: CS(=O)(=O)N1CCC2(CC1)CN(c1ccc(-n3ncc4cc(F)c(O)c(F)c43)cc1)C2 ; CS(=O)(=O)N1CC2(CN(c3ccc(-n4ncc5cc(F)c(O)c(F)c54)cc3)C2)C1 ; CC1(C)CCN(c2ccc(-n3nnc4cc(F)c(O)c(F)c43)cc2)CC1 ; CS(=O)(=O)NC1CN(c2ccc(-n3ncc4cc(F)c(O)cc43)cc2)C1] 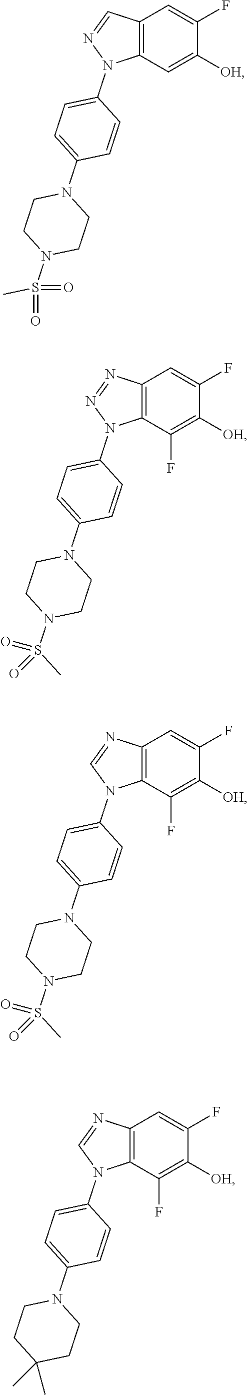 [SMILES: CS(=O)(=O)N1CCN(c2ccc(-n3ncc4cc(F)c(O)cc43)cc2)CC1 ; CS(=O)(=O)N1CCN(c2ccc(-n3nnc4cc(F)c(O)c(F)c43)cc2)CC1 ; CS(=O)(=O)N1CCN(c2ccc(-n3cnc4cc(F)c(O)c(F)c43)cc2)CC1 ; CC1(C)CCN(c2ccc(-n3cnc4cc(F)c(O)c(F)c43)cc2)CC1] 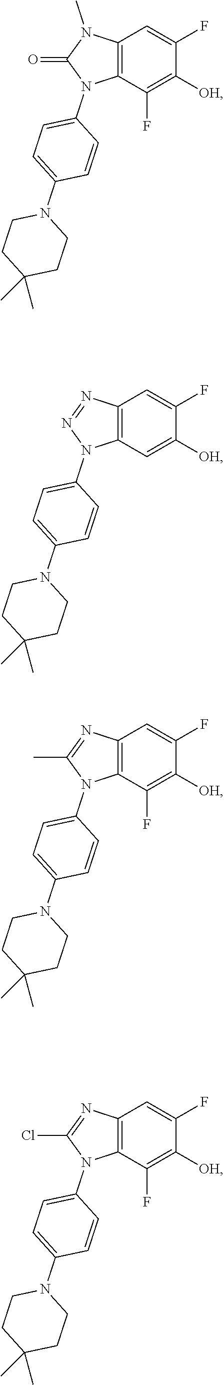 [SMILES: Cn1c(=O)n(-c2ccc(N3CCC(C)(C)CC3)cc2)c2c(F)c(O)c(F)cc21 ; CC1(C)CCN(c2ccc(-n3nnc4cc(F)c(O)cc43)cc2)CC1 ; Cc1nc2cc(F)c(O)c(F)c2n1-c1ccc(N2CCC(C)(C)CC2)cc1 ; CC1(C)CCN(c2ccc(-n3c(Cl)nc4cc(F)c(O)c(F)c43)cc2)CC1] 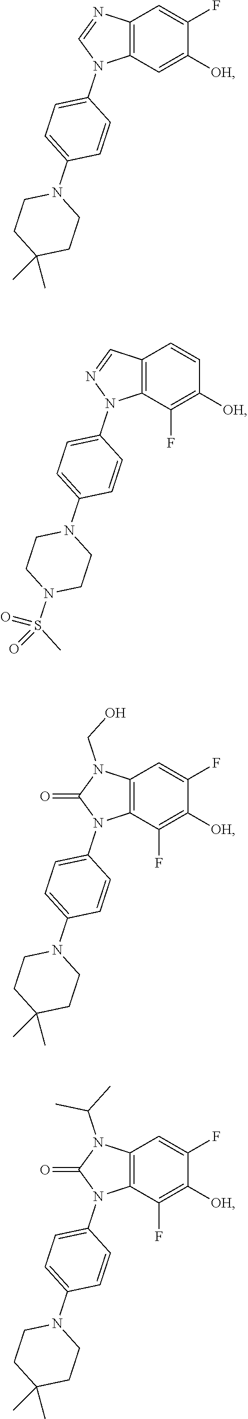 [SMILES: CC1(C)CCN(c2ccc(-n3cnc4cc(F)c(O)cc43)cc2)CC1 ; CS(=O)(=O)N1CCN(c2ccc(-n3ncc4ccc(O)c(F)c43)cc2)CC1 ; CC1(C)CCN(c2ccc(-n3c(=O)n(CO)c4cc(F)c(O)c(F)c43)cc2)CC1 ; CC(C)n1c(=O)n(-c2ccc(N3CCC(C)(C)CC3)cc2)c2c(F)c(O)c(F)cc21] 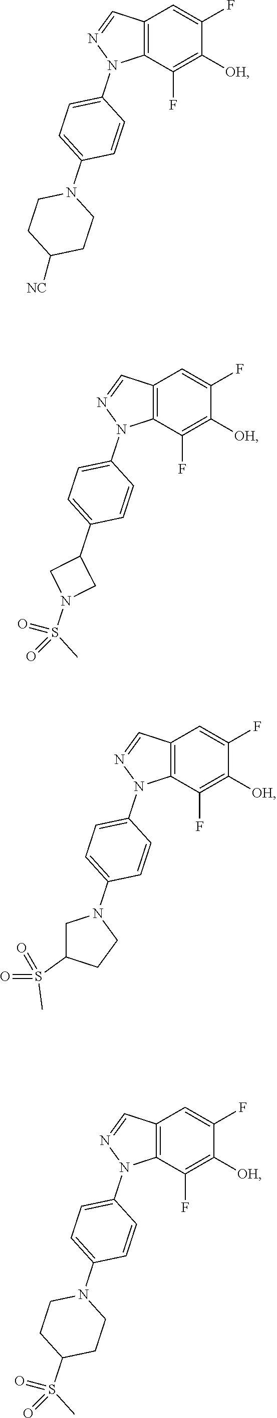 [SMILES: N#CC1CCN(c2ccc(-n3ncc4cc(F)c(O)c(F)c43)cc2)CC1 ; CS(=O)(=O)N1CC(c2ccc(-n3ncc4cc(F)c(O)c(F)c43)cc2)C1 ; CS(=O)(=O)C1CCN(c2ccc(-n3ncc4cc(F)c(O)c(F)c43)cc2)C1 ; CS(=O)(=O)C1CCN(c2ccc(-n3ncc4cc(F)c(O)c(F)c43)cc2)CC1] 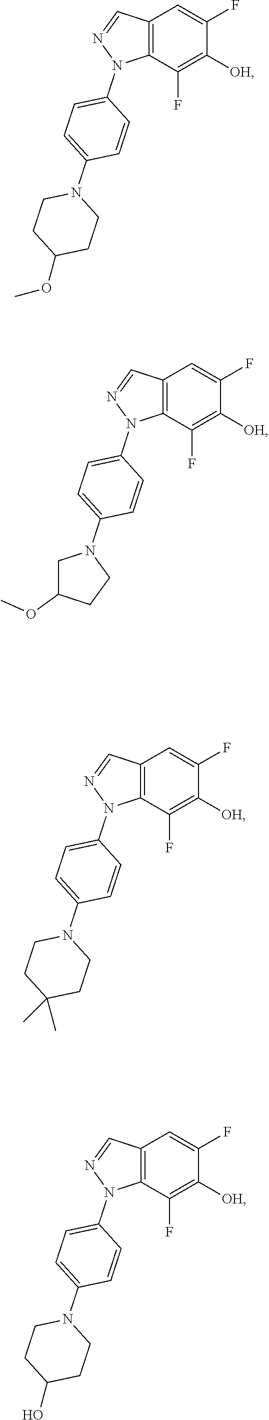 [SMILES: COC1CCN(c2ccc(-n3ncc4cc(F)c(O)c(F)c43)cc2)CC1 ; COC1CCN(c2ccc(-n3ncc4cc(F)c(O)c(F)c43)cc2)C1 ; CC1(C)CCN(c2ccc(-n3ncc4cc(F)c(O)c(F)c43)cc2)CC1 ; Oc1c(F)cc2cnn(-c3ccc(N4CCC(O)CC4)cc3)c2c1F] 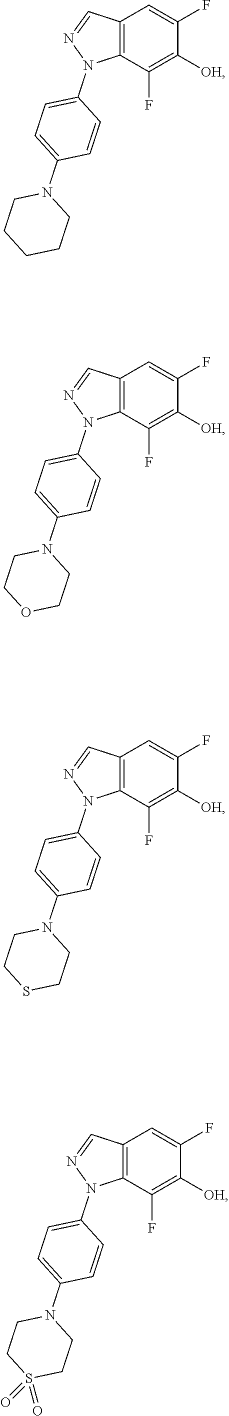 [SMILES: Oc1c(F)cc2cnn(-c3ccc(N4CCCCC4)cc3)c2c1F ; Oc1c(F)cc2cnn(-c3ccc(N4CCOCC4)cc3)c2c1F ; Oc1c(F)cc2cnn(-c3ccc(N4CCSCC4)cc3)c2c1F ; O=S1(=O)CCN(c2ccc(-n3ncc4cc(F)c(O)c(F)c43)cc2)CC1] 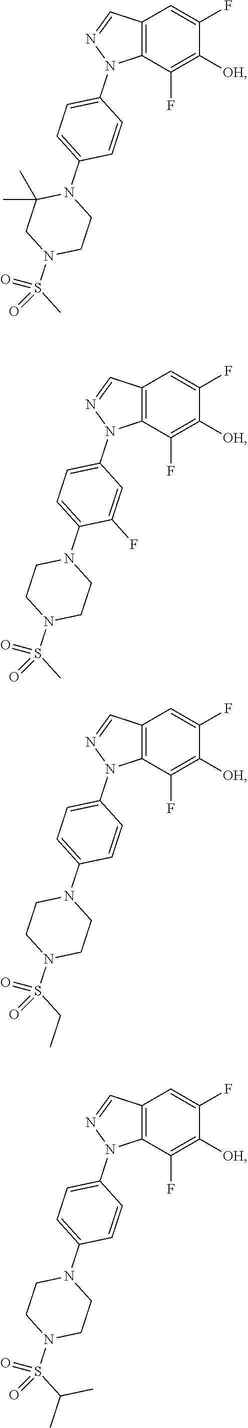 [SMILES: CC1(C)CN(S(C)(=O)=O)CCN1c1ccc(-n2ncc3cc(F)c(O)c(F)c32)cc1 ; CS(=O)(=O)N1CCN(c2ccc(-n3ncc4cc(F)c(O)c(F)c43)cc2F)CC1 ; CCS(=O)(=O)N1CCN(c2ccc(-n3ncc4cc(F)c(O)c(F)c43)cc2)CC1 ; CC(C)S(=O)(=O)N1CCN(c2ccc(-n3ncc4cc(F)c(O)c(F)c43)cc2)CC1] 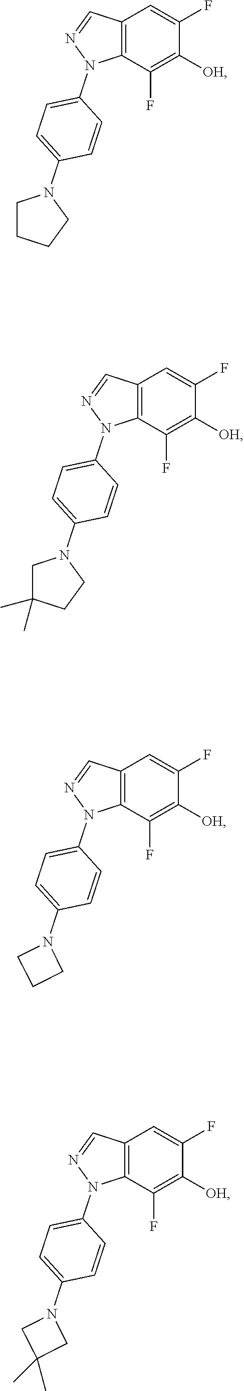 [SMILES: Oc1c(F)cc2cnn(-c3ccc(N4CCCC4)cc3)c2c1F ; CC1(C)CCN(c2ccc(-n3ncc4cc(F)c(O)c(F)c43)cc2)C1 ; Oc1c(F)cc2cnn(-c3ccc(N4CCC4)cc3)c2c1F ; CC1(C)CN(c2ccc(-n3ncc4cc(F)c(O)c(F)c43)cc2)C1] 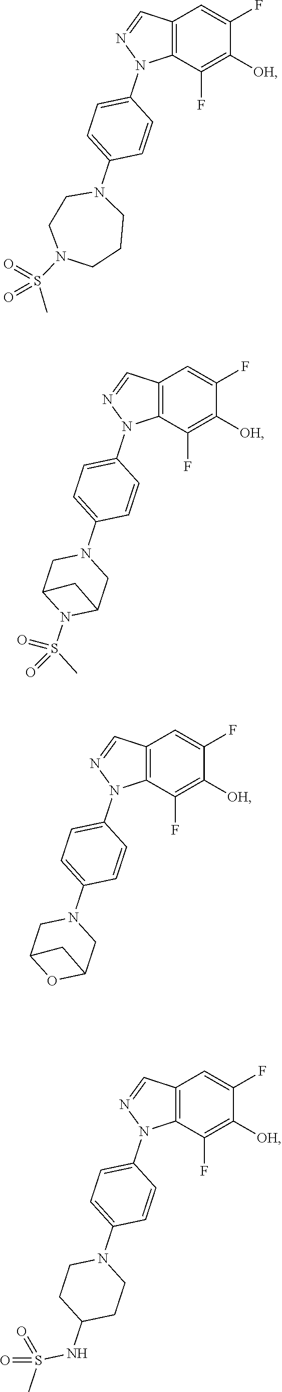 [SMILES: CS(=O)(=O)N1CCCN(c2ccc(-n3ncc4cc(F)c(O)c(F)c43)cc2)CC1 ; CS(=O)(=O)N1C2CC1CN(c1ccc(-n3ncc4cc(F)c(O)c(F)c43)cc1)C2 ; Oc1c(F)cc2cnn(-c3ccc(N4CC5CC(C4)O5)cc3)c2c1F ; CS(=O)(=O)NC1CCN(c2ccc(-n3ncc4cc(F)c(O)c(F)c43)cc2)CC1] 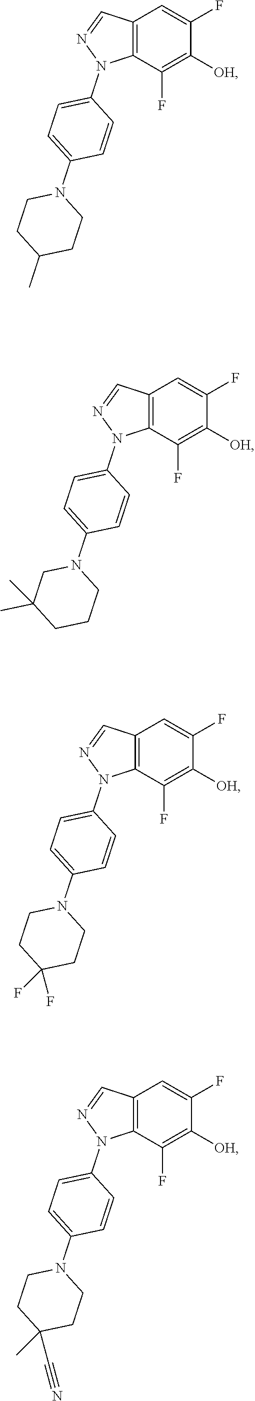 [SMILES: CC1CCN(c2ccc(-n3ncc4cc(F)c(O)c(F)c43)cc2)CC1 ; CC1(C)CCCN(c2ccc(-n3ncc4cc(F)c(O)c(F)c43)cc2)C1 ; Oc1c(F)cc2cnn(-c3ccc(N4CCC(F)(F)CC4)cc3)c2c1F ; CC1(C#N)CCN(c2ccc(-n3ncc4cc(F)c(O)c(F)c43)cc2)CC1] 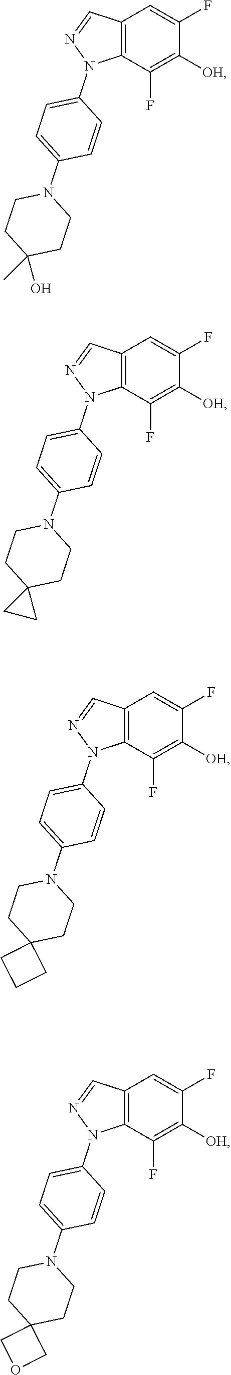 [SMILES: CC1(O)CCN(c2ccc(-n3ncc4cc(F)c(O)c(F)c43)cc2)CC1 ; Oc1c(F)cc2cnn(-c3ccc(N4CCC5(CC4)CC5)cc3)c2c1F ; Oc1c(F)cc2cnn(-c3ccc(N4CCC5(CCC5)CC4)cc3)c2c1F ; Oc1c(F)cc2cnn(-c3ccc(N4CCC5(CC4)COC5)cc3)c2c1F] 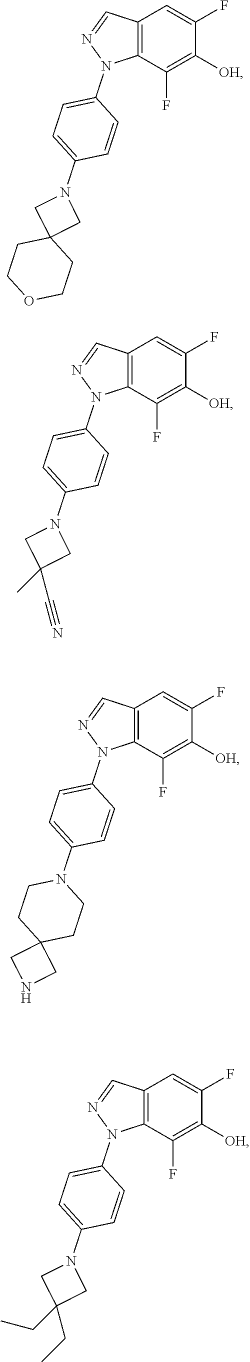 [SMILES: Oc1c(F)cc2cnn(-c3ccc(N4CC5(CCOCC5)C4)cc3)c2c1F ; CC1(C#N)CN(c2ccc(-n3ncc4cc(F)c(O)c(F)c43)cc2)C1 ; Oc1c(F)cc2cnn(-c3ccc(N4CCC5(CC4)CNC5)cc3)c2c1F ; CCC1(CC)CN(c2ccc(-n3ncc4cc(F)c(O)c(F)c43)cc2)C1] 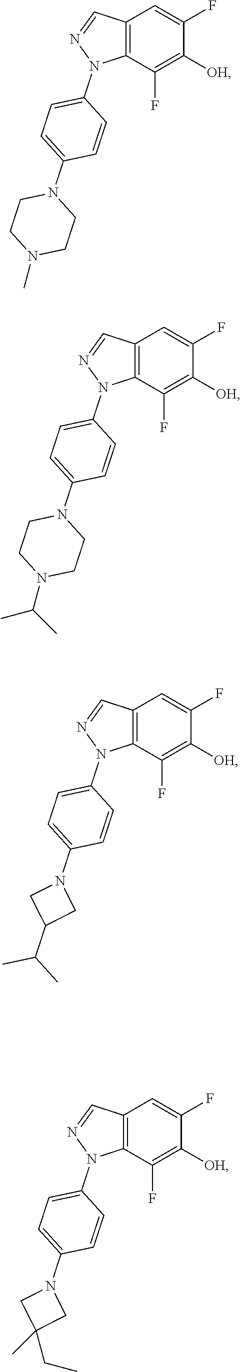 [SMILES: CN1CCN(c2ccc(-n3ncc4cc(F)c(O)c(F)c43)cc2)CC1 ; CC(C)N1CCN(c2ccc(-n3ncc4cc(F)c(O)c(F)c43)cc2)CC1 ; CC(C)C1CN(c2ccc(-n3ncc4cc(F)c(O)c(F)c43)cc2)C1 ; CCC1(C)CN(c2ccc(-n3ncc4cc(F)c(O)c(F)c43)cc2)C1] 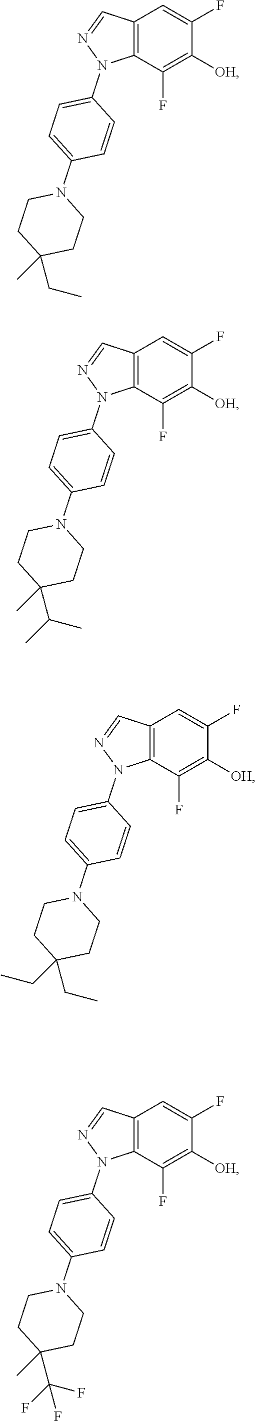 [SMILES: CCC1(C)CCN(c2ccc(-n3ncc4cc(F)c(O)c(F)c43)cc2)CC1 ; CC(C)C1(C)CCN(c2ccc(-n3ncc4cc(F)c(O)c(F)c43)cc2)CC1 ; CCC1(CC)CCN(c2ccc(-n3ncc4cc(F)c(O)c(F)c43)cc2)CC1 ; CC1(C(F)(F)F)CCN(c2ccc(-n3ncc4cc(F)c(O)c(F)c43)cc2)CC1] 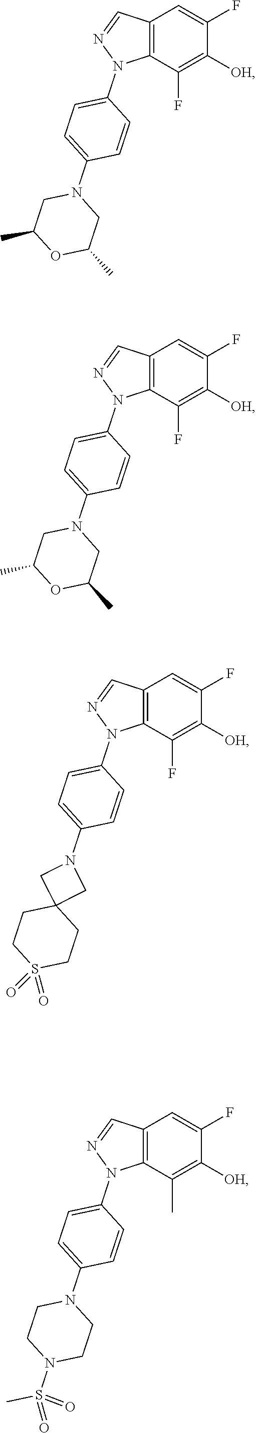 [SMILES: C[C@H]1CN(c2ccc(-n3ncc4cc(F)c(O)c(F)c43)cc2)C[C@H](C)O1 ; C[C@@H]1CN(c2ccc(-n3ncc4cc(F)c(O)c(F)c43)cc2)C[C@@H](C)O1 ; O=S1(=O)CCC2(CC1)CN(c1ccc(-n3ncc4cc(F)c(O)c(F)c43)cc1)C2 ; Cc1c(O)c(F)cc2cnn(-c3ccc(N4CCN(S(C)(=O)=O)CC4)cc3)c12] 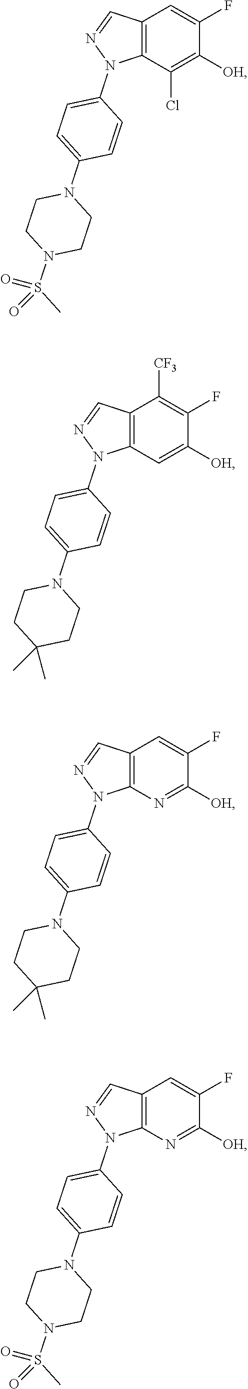 [SMILES: CS(=O)(=O)N1CCN(c2ccc(-n3ncc4cc(F)c(O)c(Cl)c43)cc2)CC1 ; CC1(C)CCN(c2ccc(-n3ncc4c(C(F)(F)F)c(F)c(O)cc43)cc2)CC1 ; CC1(C)CCN(c2ccc(-n3ncc4cc(F)c(O)nc43)cc2)CC1 ; CS(=O)(=O)N1CCN(c2ccc(-n3ncc4cc(F)c(O)nc43)cc2)CC1] 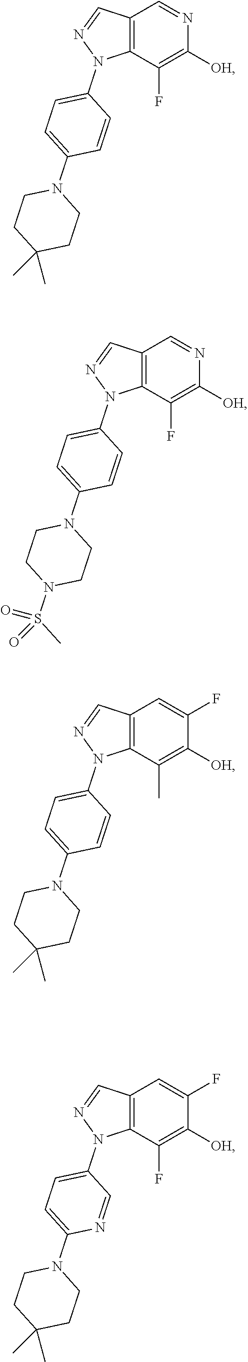 [SMILES: CC1(C)CCN(c2ccc(-n3ncc4cnc(O)c(F)c43)cc2)CC1 ; CS(=O)(=O)N1CCN(c2ccc(-n3ncc4cnc(O)c(F)c43)cc2)CC1 ; Cc1c(O)c(F)cc2cnn(-c3ccc(N4CCC(C)(C)CC4)cc3)c12 ; CC1(C)CCN(c2ccc(-n3ncc4cc(F)c(O)c(F)c43)cn2)CC1] 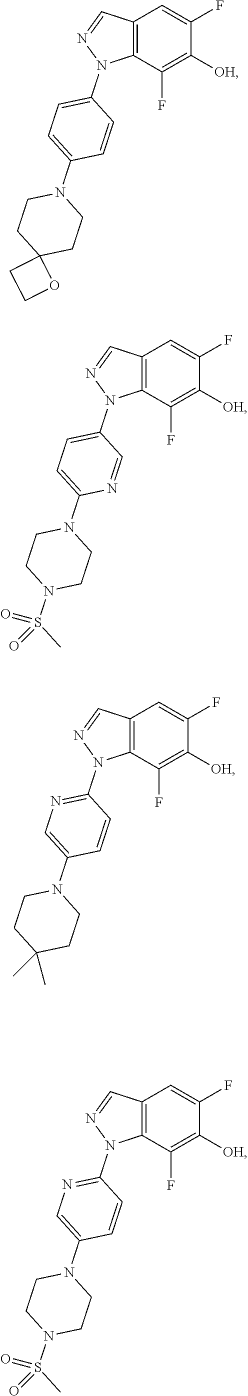 [SMILES: Oc1c(F)cc2cnn(-c3ccc(N4CCC5(CCO5)CC4)cc3)c2c1F ; CS(=O)(=O)N1CCN(c2ccc(-n3ncc4cc(F)c(O)c(F)c43)cn2)CC1 ; CC1(C)CCN(c2ccc(-n3ncc4cc(F)c(O)c(F)c43)nc2)CC1 ; CS(=O)(=O)N1CCN(c2ccc(-n3ncc4cc(F)c(O)c(F)c43)nc2)CC1] 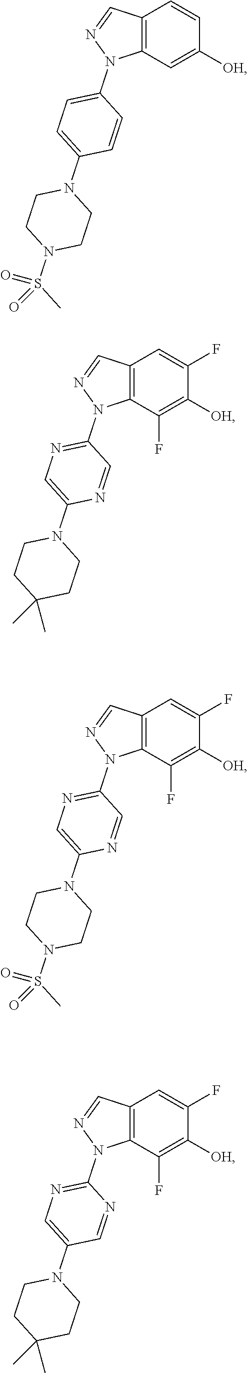 [SMILES: CS(=O)(=O)N1CCN(c2ccc(-n3ncc4ccc(O)cc43)cc2)CC1 ; CC1(C)CCN(c2cnc(-n3ncc4cc(F)c(O)c(F)c43)cn2)CC1 ; CS(=O)(=O)N1CCN(c2cnc(-n3ncc4cc(F)c(O)c(F)c43)cn2)CC1 ; CC1(C)CCN(c2cnc(-n3ncc4cc(F)c(O)c(F)c43)nc2)CC1] 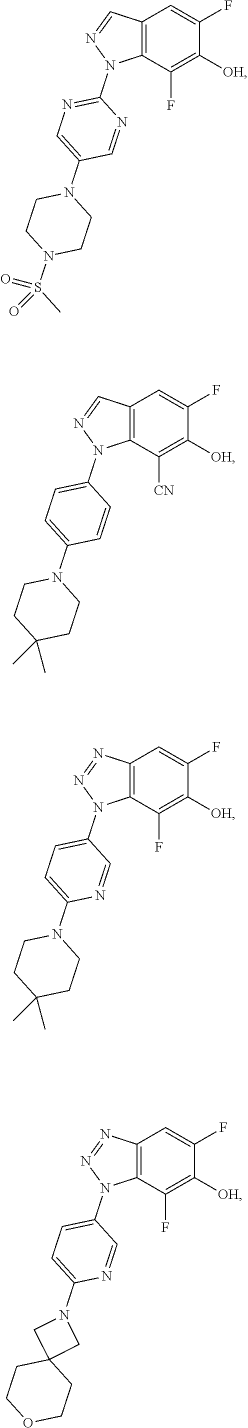 [SMILES: CS(=O)(=O)N1CCN(c2cnc(-n3ncc4cc(F)c(O)c(F)c43)nc2)CC1 ; CC1(C)CCN(c2ccc(-n3ncc4cc(F)c(O)c(C#N)c43)cc2)CC1 ; CC1(C)CCN(c2ccc(-n3nnc4cc(F)c(O)c(F)c43)cn2)CC1 ; Oc1c(F)cc2nnn(-c3ccc(N4CC5(CCOCC5)C4)nc3)c2c1F] 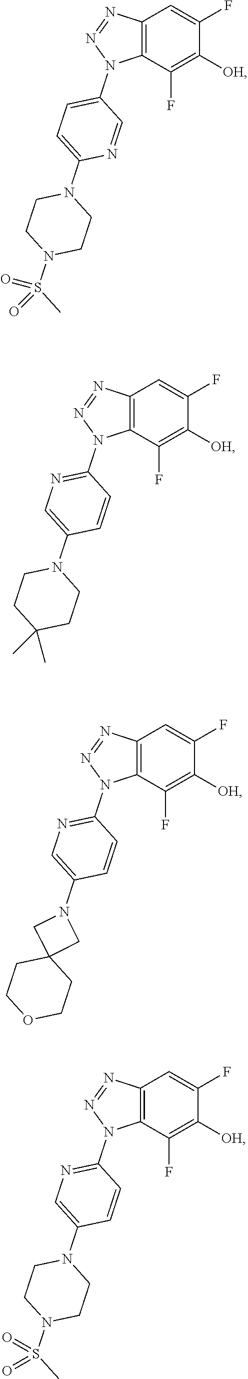 [SMILES: CS(=O)(=O)N1CCN(c2ccc(-n3nnc4cc(F)c(O)c(F)c43)cn2)CC1 ; CC1(C)CCN(c2ccc(-n3nnc4cc(F)c(O)c(F)c43)nc2)CC1 ; Oc1c(F)cc2nnn(-c3ccc(N4CC5(CCOCC5)C4)cn3)c2c1F ; CS(=O)(=O)N1CCN(c2ccc(-n3nnc4cc(F)c(O)c(F)c43)nc2)CC1] 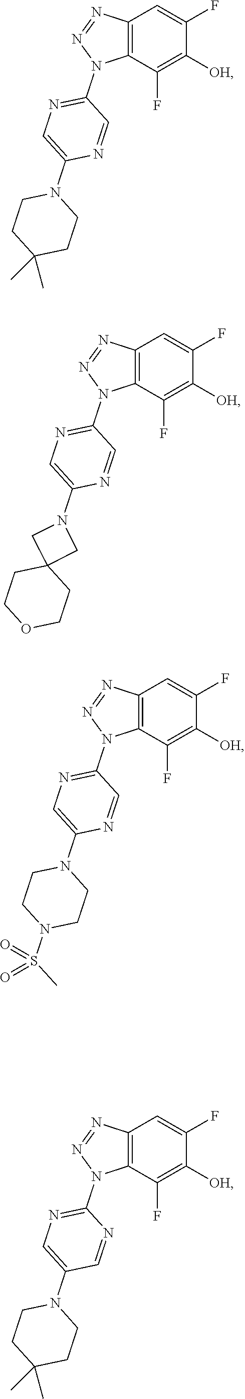 [SMILES: CC1(C)CCN(c2cnc(-n3nnc4cc(F)c(O)c(F)c43)cn2)CC1 ; Oc1c(F)cc2nnn(-c3cnc(N4CC5(CCOCC5)C4)cn3)c2c1F ; CS(=O)(=O)N1CCN(c2cnc(-n3nnc4cc(F)c(O)c(F)c43)cn2)CC1 ; CC1(C)CCN(c2cnc(-n3nnc4cc(F)c(O)c(F)c43)nc2)CC1] 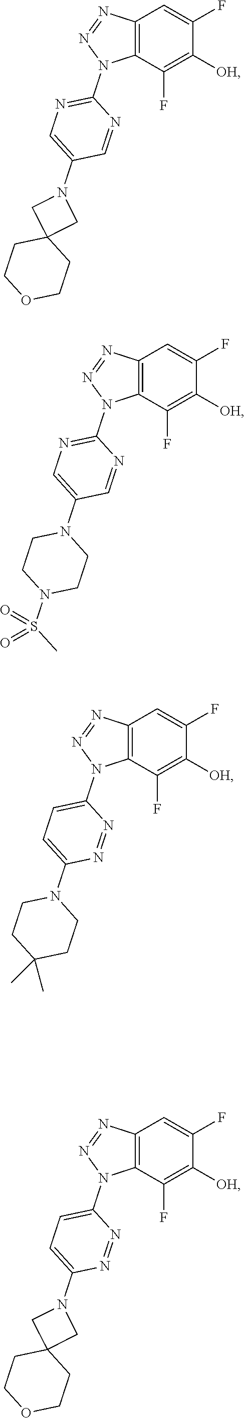 [SMILES: Oc1c(F)cc2nnn(-c3ncc(N4CC5(CCOCC5)C4)cn3)c2c1F ; CS(=O)(=O)N1CCN(c2cnc(-n3nnc4cc(F)c(O)c(F)c43)nc2)CC1 ; CC1(C)CCN(c2ccc(-n3nnc4cc(F)c(O)c(F)c43)nn2)CC1 ; Oc1c(F)cc2nnn(-c3ccc(N4CC5(CCOCC5)C4)nn3)c2c1F] 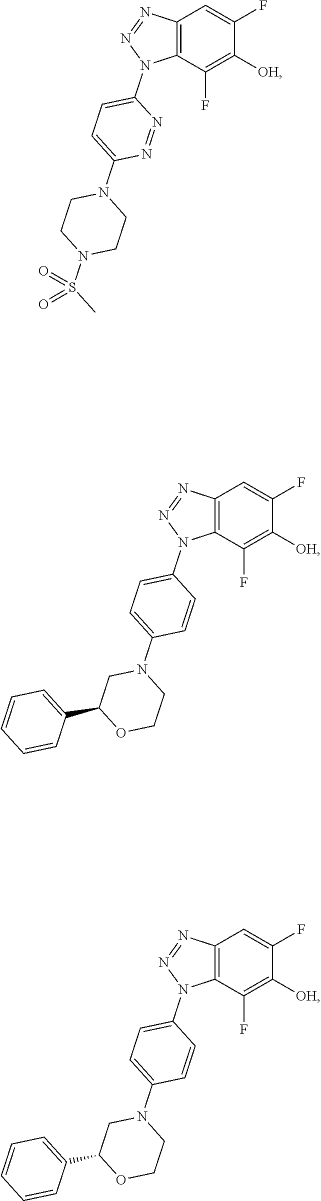 [SMILES: CS(=O)(=O)N1CCN(c2ccc(-n3nnc4cc(F)c(O)c(F)c43)nn2)CC1 ; Oc1c(F)cc2nnn(-c3ccc(N4CCO[C@@H](c5ccccc5)C4)cc3)c2c1F ; Oc1c(F)cc2nnn(-c3ccc(N4CCO[C@H](c5ccccc5)C4)cc3)c2c1F] 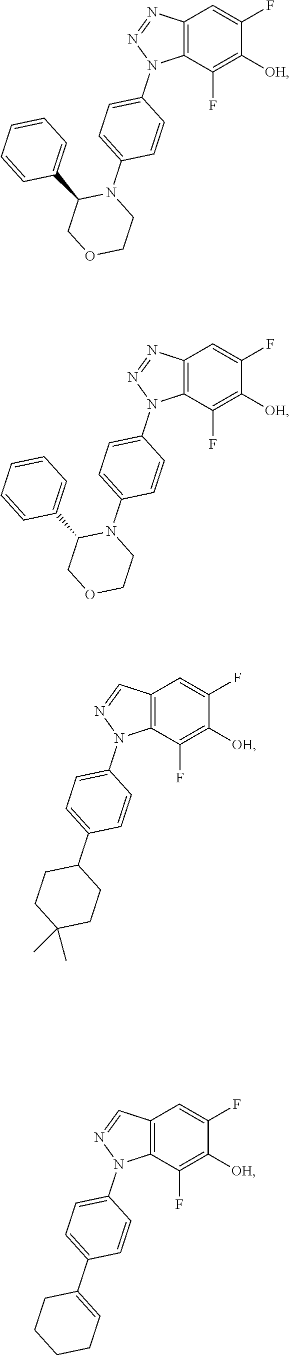 [SMILES: Oc1c(F)cc2nnn(-c3ccc(N4CCOC[C@H]4c4ccccc4)cc3)c2c1F ; Oc1c(F)cc2nnn(-c3ccc(N4CCOC[C@@H]4c4ccccc4)cc3)c2c1F ; CC1(C)CCC(c2ccc(-n3ncc4cc(F)c(O)c(F)c43)cc2)CC1 ; Oc1c(F)cc2cnn(-c3ccc(C4=CCCCC4)cc3)c2c1F] 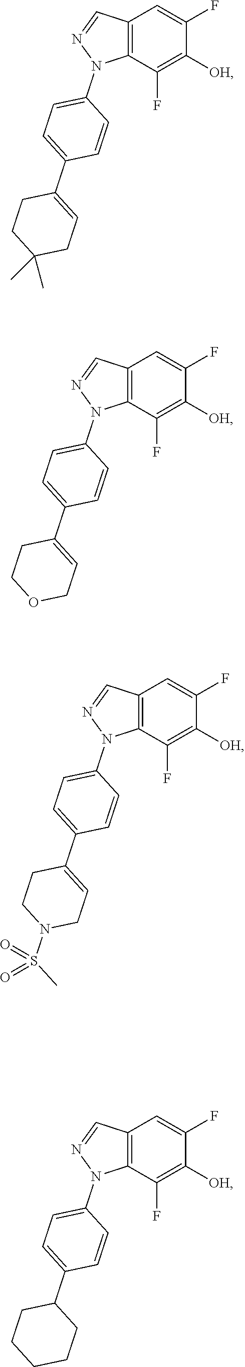 [SMILES: CC1(C)CC=C(c2ccc(-n3ncc4cc(F)c(O)c(F)c43)cc2)CC1 ; Oc1c(F)cc2cnn(-c3ccc(C4=CCOCC4)cc3)c2c1F ; CS(=O)(=O)N1CC=C(c2ccc(-n3ncc4cc(F)c(O)c(F)c43)cc2)CC1 ; Oc1c(F)cc2cnn(-c3ccc(C4CCCCC4)cc3)c2c1F] 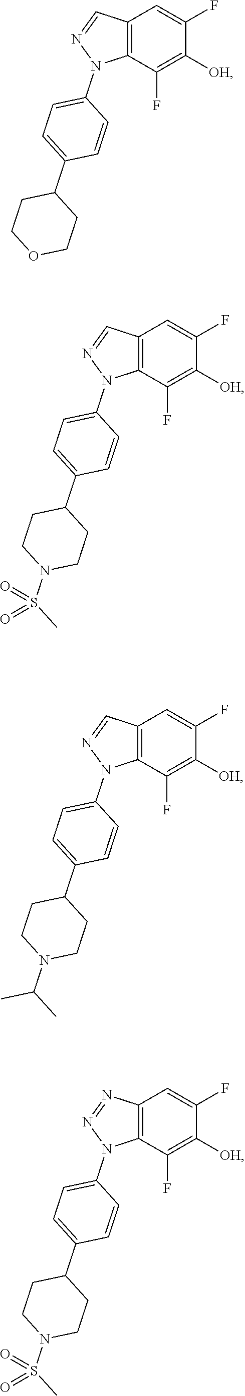 [SMILES: Oc1c(F)cc2cnn(-c3ccc(C4CCOCC4)cc3)c2c1F ; CS(=O)(=O)N1CCC(c2ccc(-n3ncc4cc(F)c(O)c(F)c43)cc2)CC1 ; CC(C)N1CCC(c2ccc(-n3ncc4cc(F)c(O)c(F)c43)cc2)CC1 ; CS(=O)(=O)N1CCC(c2ccc(-n3nnc4cc(F)c(O)c(F)c43)cc2)CC1] 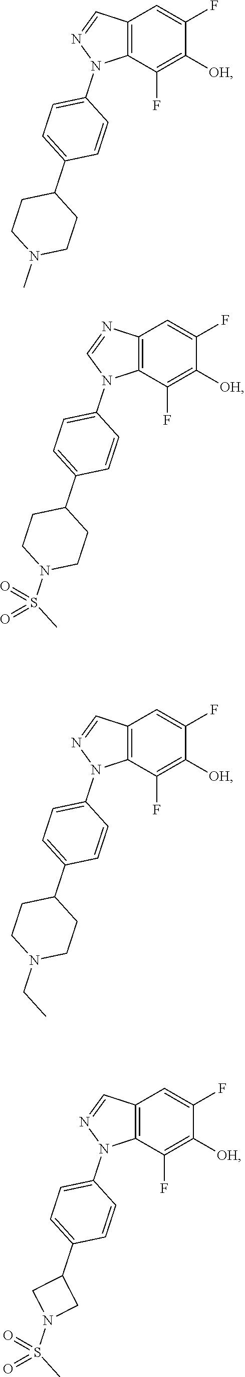 [SMILES: CN1CCC(c2ccc(-n3ncc4cc(F)c(O)c(F)c43)cc2)CC1 ; CS(=O)(=O)N1CCC(c2ccc(-n3cnc4cc(F)c(O)c(F)c43)cc2)CC1 ; CCN1CCC(c2ccc(-n3ncc4cc(F)c(O)c(F)c43)cc2)CC1 ; CS(=O)(=O)N1CC(c2ccc(-n3ncc4cc(F)c(O)c(F)c43)cc2)C1] 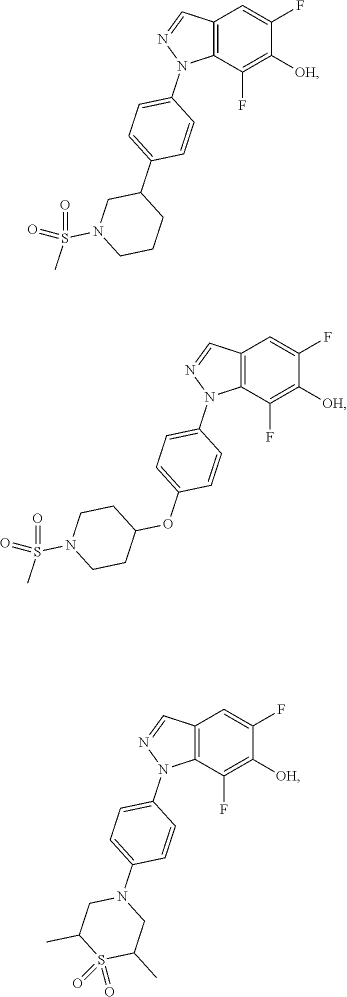 [SMILES: CS(=O)(=O)N1CCCC(c2ccc(-n3ncc4cc(F)c(O)c(F)c43)cc2)C1 ; CS(=O)(=O)N1CCC(Oc2ccc(-n3ncc4cc(F)c(O)c(F)c43)cc2)CC1 ; CC1CN(c2ccc(-n3ncc4cc(F)c(O)c(F)c43)cc2)CC(C)S1(=O)=O] 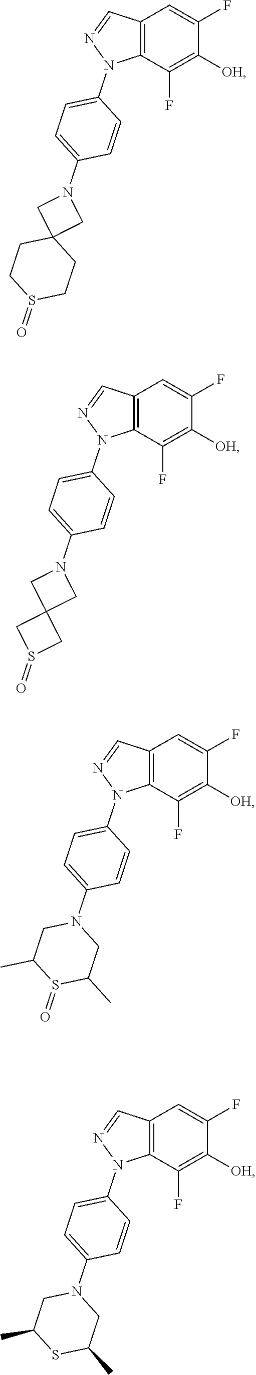 [SMILES: O=S1CCC2(CC1)CN(c1ccc(-n3ncc4cc(F)c(O)c(F)c43)cc1)C2 ; O=S1CC2(CN(c3ccc(-n4ncc5cc(F)c(O)c(F)c54)cc3)C2)C1 ; CC1CN(c2ccc(-n3ncc4cc(F)c(O)c(F)c43)cc2)CC(C)S1=O ; C[C@@H]1CN(c2ccc(-n3ncc4cc(F)c(O)c(F)c43)cc2)C[C@H](C)S1] 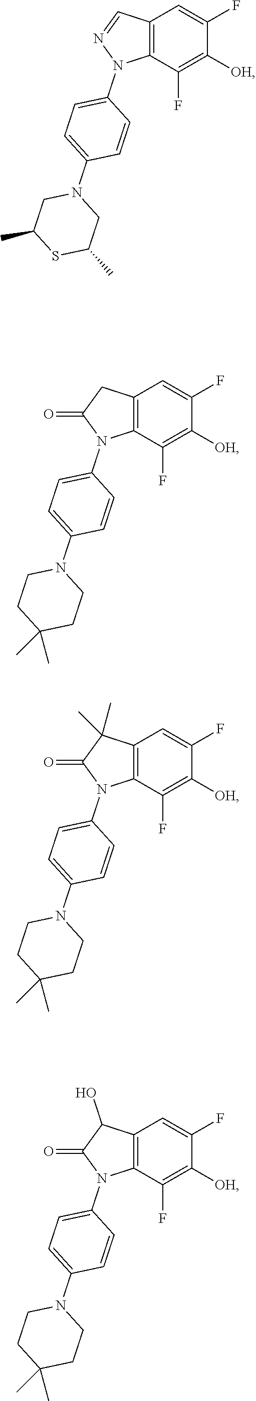 [SMILES: C[C@H]1CN(c2ccc(-n3ncc4cc(F)c(O)c(F)c43)cc2)C[C@H](C)S1 ; CC1(C)CCN(c2ccc(N3C(=O)Cc4cc(F)c(O)c(F)c43)cc2)CC1 ; CC1(C)CCN(c2ccc(N3C(=O)C(C)(C)c4cc(F)c(O)c(F)c43)cc2)CC1 ; CC1(C)CCN(c2ccc(N3C(=O)C(O)c4cc(F)c(O)c(F)c43)cc2)CC1] 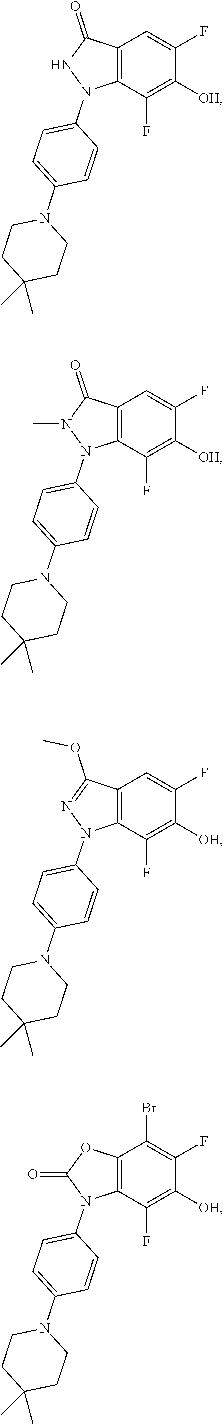 [SMILES: CC1(C)CCN(c2ccc(-n3[nH]c(=O)c4cc(F)c(O)c(F)c43)cc2)CC1 ; Cn1c(=O)c2cc(F)c(O)c(F)c2n1-c1ccc(N2CCC(C)(C)CC2)cc1 ; COc1nn(-c2ccc(N3CCC(C)(C)CC3)cc2)c2c(F)c(O)c(F)cc12 ; CC1(C)CCN(c2ccc(-n3c(=O)oc4c(Br)c(F)c(O)c(F)c43)cc2)CC1] 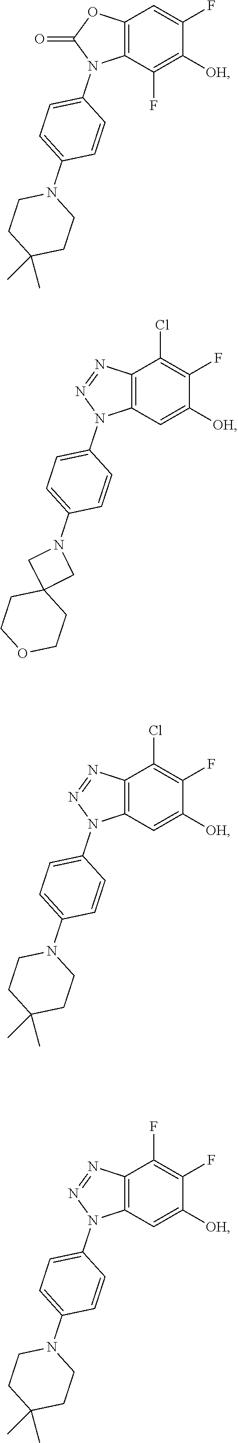 [SMILES: CC1(C)CCN(c2ccc(-n3c(=O)oc4cc(F)c(O)c(F)c43)cc2)CC1 ; Oc1cc2c(nnn2-c2ccc(N3CC4(CCOCC4)C3)cc2)c(Cl)c1F ; CC1(C)CCN(c2ccc(-n3nnc4c(Cl)c(F)c(O)cc43)cc2)CC1 ; CC1(C)CCN(c2ccc(-n3nnc4c(F)c(F)c(O)cc43)cc2)CC1] 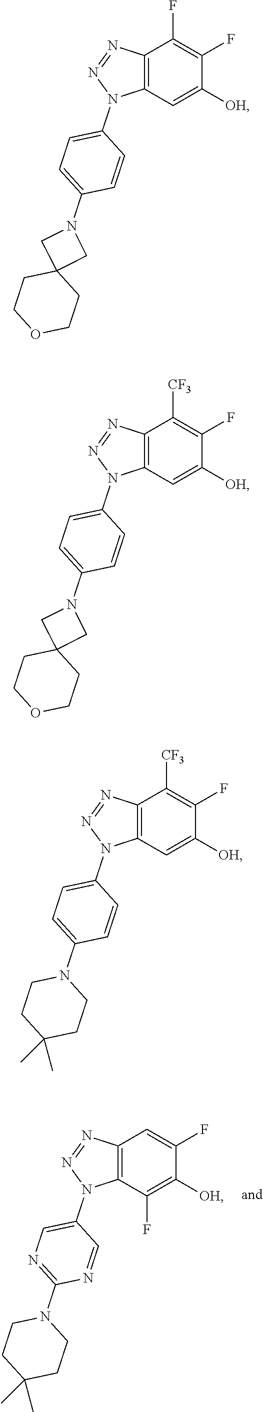 [SMILES: Oc1cc2c(nnn2-c2ccc(N3CC4(CCOCC4)C3)cc2)c(F)c1F ; Oc1cc2c(nnn2-c2ccc(N3CC4(CCOCC4)C3)cc2)c(C(F)(F)F)c1F ; CC1(C)CCN(c2ccc(-n3nnc4c(C(F)(F)F)c(F)c(O)cc43)cc2)CC1 ; CC1(C)CCN(c2ncc(-n3nnc4cc(F)c(O)c(F)c43)cn2)CC1] 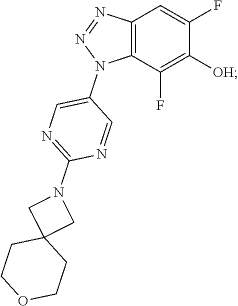 [SMILES: Oc1c(F)cc2nnn(-c3cnc(N4CC5(CCOCC5)C4)nc3)c2c1F]
or a pharmaceutically acceptable salt or solvate thereof.

42. A pharmaceutical composition comprising a compound of any one of claims 1-41, or a pharmaceutically acceptable salt or solvate thereof, and at least one pharmaceutically acceptable excipient.

43. The pharmaceutical composition of claim 42, wherein the pharmaceutical composition is formulated for administration to a mammal by intravenous administration, subcutaneous administration, oral administration, inhalation, nasal administration, dermal administration, or ophthalmic administration.

44. The pharmaceutical composition of claim 42, wherein the pharmaceutical composition is in the form of a tablet, a pill, a capsule, a liquid, a suspension, a gel, a dispersion, a solution, an emulsion, an ointment, or a lotion.

45. A method of treating or preventing a liver disease or condition in a mammal, comprising administering to the mammal a compound of any one of claims 1-41, or a pharmaceutically acceptable salt or solvate thereof.

46. The method of claim 45, wherein the liver disease or condition is an alcoholic liver disease or condition.

47. The method of claim 45, wherein the liver disease or condition is a nonalcoholic liver disease or condition.

48. The method of claim 45, wherein the liver disease or condition is liver inflammation, fatty liver (steatosis), liver fibrosis, hepatitis, cirrhosis, hepatocellular carcinoma, or combinations thereof.

49. The method of claim 45, wherein the liver disease or condition is primary biliary cirrhosis, primary sclerosing cholangitis, cholestasis, nonalcoholic steatohepatitis (NASH), nonalcoholic fatty liver disease (NAFLD), or combinations thereof.

50. A method of treating or preventing a disease or condition in a mammal that would benefit from treatment with an HSD17B13 inhibitor, comprising administering to the mammal a compound of any one of claims 1-41, or a pharmaceutically acceptable salt or solvate thereof.

51. The method of claim 50, wherein the disease or condition in the mammal that would benefit from treatment with an HSD17B13 inhibitor mammal is a liver disease or condition as described in claim 48 or claim 49.

52. A method of modulating hydroxysteroid 17β-dehydrogenase 13 (HSD17B13) activity in a mammal comprising administering to the mammal a compound of any one of claims 1-41, or a pharmaceutically acceptable salt or solvate thereof.

53. The method of claim 52, wherein modulating comprises inhibiting HSD17B13 activity.

54. The method of claim 52 or claim 53, wherein the mammal has a liver disease or condition as described in claim 48 or claim 49.
